(19) United States
(12) Patent Application Publication
(10) Pub. No.: US 2025/0127728 A1
(43) Pub. Date: Apr. 24, 2025

(54) Constrained Ionizable Cationic Lipids and Lipid Nanoparticles

(71) Applicant: CAPSTAN THERAPEUTICS, INC., San Diego, CA (US)

(72) Inventors: Priya Prakash Karmali, San Diego, CA (US); Steven Tanis, Carlsbad, CA (US); Yanjie Bao, San Diego, CA (US)

(21) Appl. No.: 18/904,951

(22) Filed: Oct. 2, 2024

Related U.S. Application Data
(60) Provisional application No. 63/588,282, filed on Oct. 5, 2023.
(60) Provisional application No. 63/632,931, filed on Apr. 11, 2024.
(60) Provisional application No. 63/654,744, filed on May 31, 2024.

(51) Int. Cl. A61K 9/51; C07D 207/12

(52) U.S. Cl. CPC A61K 9/5123; C07D 207/12

(57) ABSTRACT

Ionizable cationic lipids, methods for synthesizing the same, intermediates useful in synthesis of the ionizable cationic lipids and methods of synthesizing the intermediates are disclosed. The ionizable cationic lipids are useful as a component of lipid nanoparticles (LNP), which in turn can be used for the delivery of nucleic acids into cells in vivo or ex vivo. LNP compositions are also disclosed, including LNP comprising a functionalized lipid to enable conjugation of a binding moiety, and targeted LNP (tLNP), that is an LNP in which a binding moiety has been conjugated to the functionalized lipid and can serve as a targeting moiety to direct the tLNP to a desired tissue or cell type.

### CROSS-REFERENCE TO RELATED APPLICATIONS

This application claims priority to U.S. provisional application No. 63/588,282, filed Oct. 5, 2023; U.S. provisional application No. 63/632,931, filed Apr. 11, 2024; and U.S. provisional application No. 63/654,744, filed May 31, 2024; the disclosures of each of which are expressly incorporated by reference herein.

### REFERENCE TO AN ELECTRONIC SEQUENCE LISTING

The instant application contains a Sequence Listing that has been submitted electronically and is hereby incorporated by reference in its entirety. The Sequence Listing was created on Sep. 27, 2024, is named “24-0221-VWO.xml”, and is 11,729 bytes in size.

### BACKGROUND

Upid formulations have been used in the laboratory for delivering nucleic acids into cells. Early formulations based on the cationic lipid 1,2-dioleoyl-3-trimethylammonium propane (DOTAP) and the ionizable, fusogenic lipid dioleoylphosphatidyl ethanolamine (DOPE) had a large partile size and were problematic when used in vivo, exhibiting too rapid clearance, tropism for the lung, and toxicity. Lipid nanoparticles (LNPs) comprising ionizable cationic lipids have been developed to address these issues to the extent that RNA-based products, such as the siRNA ONPATTRO® and two mRNA-based SARS-CoV-2 vaccines have received regulatory approval and entered the marketplace.

However, there is limited ability to control which tissues or cells take up the LNP once administered. LNP administered intravenously are taken up primarily in the liver, lung, or spleen depending to a significant degree on net charge and particle size. It is possible to direct >90% of LNP to the liver by a combination of formulation and intravenous administration, for example. Intramuscular administration can provide a clinically useful level of local delivery and expression. LNP can be redirected to other tissues or cell types by conjugating to the LNP a binding moiety with specificity for the target tissue or cell type, for example, conjugating an antibody to an LNP (see, e.g., Endsley and Ho, J. Acquir. Immune Defic. Syndr. 61:417, 2012; Ramishetti et al., ACS Nano 9:6706, 2015; Veiga et al., Nat. Comms. 9:4493, 2018; U.S. Pat. No. 10,920,246). Nonetheless, avoiding uptake by the liver remains a challenge. Moreover, with current systems only a minor portion of the encapsulated nucleic acid is successfully delivered to the cells of interest and into the cytoplasm. Current formulations may release only 2-5% of the administered RNA into the cytoplasm (see for example Gilleron et al., 2013, Nat. Biotechnol. 31:638-646 and Munson et al., 2021, Commun. Biol. 4:211-224). There are remaining issues of off-target delivery, poor efficiency of release of nucleic acid into the cytoplasm, and toxicity associated with accumulation of the component lipids.

Thus, there exists a need to address issues of off-target delivery, poor efficiency of release of nucleic acid into the cytoplasm, and toxicity associated with accumulation of the component lipids.

### SUMMARY

This disclosure is directed towards fulfilling the needs for addressing issues of off-target delivery, poor efficiency of release of therapeutic agents and provides further related advantages.

In certain aspects, this disclosure provides constrained ionizable cationic lipids having a structure of formula M2 set forth herein.

In some embodiments, this disclosure provides ionizable cationic lipids CICL-207, CICL-215, CICL-216, CICL-217, CICL-218, CICL-219, CICL-220, CICL-221, CICL-222, CICL-223, CICL-224, CICL-225, CICL-238, CICL-239, CICL-242, CICL-243, CICL-244, CICL-245, CICL-246, CICL-247, CICL-248, and CICL-249 set forth herein.

In certain aspects, this disclosure provides methods of synthesizing ionizable cationic lipids as described herein, e.g., CICL-207, CICL-215, CICL-216, CICL-217, CICL-218, CICL-219, CICL-220, CICL-221, CICL-222, CICL-223, CICL-224, CICL-225, CICL-238, CICL-239, CICL-242, CICL-243, CICL-244, CICL-245, CICL-246, CICL-247, CICL-248, and CICL-249 set forth herein.

In certain aspects, this disclosure provides lipid nanoparticles (LNPs) and targeted lipid nanoparticles (tLNPs) incorporating the ionizable cationic lipids disclosed in this disclosure.

In certain aspects, this disclosure provides methods for preparing LNPs and tLNPs as described herein.

In certain embodiments, this disclosure provides methods of delivering a biologically active payload (e.g., nucleic acid molecules encoding a therapeutic agent) into a cell comprising contacting the cell with an LNP or tLNP of this disclosure.

These and other features, objects, and advantages of this disclosure will become better understood from the description that follows. In the description, reference is made to the accompanying drawings, which form a part hereof and in which there is shown by way of illustration, not limitation, embodiments of this disclosure. The description of preferred embodiments is not intended to limit this disclosure from covering all modifications, equivalents, and alternatives. Reference should therefore be made to the claims recited herein for interpreting the scope of the disclosure.

### BRIEF DESCRIPTION OF THE DRAWINGS

The disclosure is better understood and features, aspects, and advantages other than those set forth above will become apparent when consideration is given to the following description of the drawings.

FIGS. 1A-1C depict the transfection rate (percentage of cells expressing the mRNA) versus expression level (as molecules of equivalent soluble fluorochrome (MESF)) for four tLNP compositions varying in the ionizable cationic lipid and/or PEG-lipid component, as indicated. The full lipid compositions are described in Table 7. Results in splenic T cells (FIG. 1A), CD45⁻ liver cells (FIG. 1B) and liver Kupffer cells (CD45⁺/CD1⁺ liver cells; FIG. 1C) from C57BL/6 mice administered the tLNPs are shown. The binding moiety of the tLNPs was an anti-CD5 antibody and the payload was an mRNA encoding mCherry.

FIGS. 2A-2C depict the transfection rate (percentage of cells expressing the mRNA) versus expression level (as molecules of equivalent soluble fluorochrome (MESF)) for two tLNP compositions varying only in the ionizable cationic lipid component, as indicated. The full lipid compositions are described in Table 7. Results in splenic T cells (FIG. 2A), CD45⁻ liver cells (FIG. 2B) and liver Kupffer cells (CD45⁺/CD11⁺ liver cells; FIG. 2C) from C57BL/mice administered the tLNPs are shown. The binding moiety of the tLNPs was an anti-CD5 antibody and the payload was an mRNA encoding mCherry.

FIGS. 3A-3B depict compiled transfection rate data (percentage of cells expressing the mRNA; FIG. 3A) and expression level (MESF; FIG. 3B) for tLNP incorporating various ionizable cationic lipids, or different amounts of ionizable cationic lipid, normalized to the result obtained for tLNP comprising 58% CICL-207 as the ionizable cationic lipid in each experiment. The binding moiety of the tLNPs was an anti-CD5 antibody and the payload was an mRNA encoding mCherry.

FIGS. 4A-4C depict transfection rate for tLNP incorporating various ionizable cationic lipids versus measured pKa of the tLNP for splenic T cells (FIG. 4A), CD45⁻ liver cells (FIG. 4B) and liver Kupffer cells (CD45⁺/CD11⁺ liver cells; FIG. 4C) from C57BL/6 mice administered the tLNPs. The full lipid compositions are described in Table 7. The binding moiety of the tLNPs was an anti-CD5 antibody and the payload was an mRNA encoding mCherry.

FIGS. 5A-5C depict the transfection rate (percentage of cells expressing the mRNA) versus expression level (as MESF or mean fluorescence intensity (MFI)) for tLNP compositions varying only in the ionizable cationic lipid component, as indicated. The full lipid composition was CICL:DSPC:CHOL:DSG-PEG(2k):DSPE-PEG(2k)-MAL{58:10:30.5:1.4:0.1}. Results in splenic T cells (FIG. 5A), CD45⁻ liver cells (FIG. 5B) and liver Kupffer cells (CD45⁺/CD11⁺ liver cells; FIG. 5C) from C57BL/6 mice administered the tLNPs are shown. The binding moiety of the tLNPs was an anti-CD5 antibody and the payload was an mRNA encoding mCherry.

FIGS. 6A-6C depict the transfection rate (percentage of cells expressing the mRNA) for tLNP comprising different amounts of ionizable cationic lipid for two ionizable cationic lipids and compensatory changes in cholesterol content. The full lipid compositions are described in Table 7. Results in splenic T cells (FIG. 6A), CD45⁻ liver cells (FIG. 6B) and liver Kupffer cells (CD45⁺/CD11⁺ liver cells; FIG. 6C) from C57BL/6 mice administered the tLNPs are shown.

FIGS. 7A-7H portray the results of studies in NCG mice engrafted with human peripheral blood mononuclear cells (PBMCs) and dosed with tLNP composition 58% CICL-1 (F9) and 58% CICL-207 (F50) targeted to CD8 and encapsulating an mRNA encoding either mCherry or an anti-CD19 CAR. FIGS. 7A-7B depict mCherry or anti-CD19 CAR engineering rate in bar graphs for CD8⁺ T cells in spleen and blood from NCG mice engrafted with human PBMCs following administration of tLNP targeted to CD8⁺ cells. Results for engineering rate in splenic T and blood cells are displayed in FIGS. 7A and 7B, respectively. FIGS. 7C-7F depict anti-CD19 CAR expression as MFI and CAR molecules per cell (based on MESH) in bar graphs for CD8⁺ T cells in spleen and blood from NCG mice engrafted with human PBMCs following administration of tLNP targeted to CD8⁺ cells. Results for CAR expression MFI in splenic T and blood cells are displayed in FIGS. 7C and 7D, respectively. Results for CAR molecules per cell in splenic T cells and blood cells are displayed in FIGS. 7E and 7F, respectively. FIGS. 7G-7H depict B cells depletion in bar graphs in spleen for 6 hours after the mice received the tLNP dose. The number of B cells per μL spleen cell suspension and percentage of B cells are displayed in FIGS. 7G and 7H respectively.

FIGS. 8A-8E present whole-animal images (FIGS. 8A-8B, prone and supine, respectively) and individual organ bioluminescence images collected at 6 and 24 hours post injection (FIGS. 8C-8D, respectively) and individual organ bioluminescence quantitation (FIG. 8E) for C57BL/6 mice administered BF1 LNPs, CD5-targeted BF1 tLNPs, F50 LNPs, or CD5-targeted F50 tLNPs at 6 hours and 24 hours after administration.

FIG. 9 depicts in vivo transfection efficiency as % mCherry positive cells in bone marrow CD34⁺ cells (left panel) and CD34⁺CD117⁺ cells (right panel) in humanized CD34⁺ NCG mice after administration/transfection with mCherry mRNA using a tLNP decorated with an anti-CD117 monoclonal antibody. The tLNPs comprised either CICL-1 (F9) or CICL-207 (F50) lipid compositions and targeted CD117⁺ hematopoietic stem and progenitor cells (HSPCs). The bars represent the mean from the results of three mice (F9 tLNP mCherry and F50 tLNP mCherry groups), or one mouse (PBS group).

FIG. 10 depicts transfection efficiency as % mCherry positive cells post CD117-targeted tLNP transfection in CD34⁺ NCG mice. The tLNPs comprised the CICL-1 (F9) or CICL-207 (F50) lipid compositions and encapsulated mCherry mRNA and targeted CD117⁺ hematopoietic stem and progenitor cells. Data is plotted for bone marrow CD34⁺CD38^{low} cells (upper left panel), CD117⁺CD34⁺CD38^{low} cells (upper right panel) and CD117⁻CD34⁺CD38^{low} cells (lower panel).

FIG. 11 depicts percent B2M (beta-2-microglobulin) knock-out in bone marrow CD34⁺ (left panel) or CD34⁺CD38^{low} in humanized CD34⁺ NCG mice after administration/transfection of CD117-targeted tLNPs encapsulating gene editing payloads (SpCas9 mRNA+sgRNA targeting B2M locus), given in 30 μg or 60 μg of the tLNPs. The tLNPs comprised either CICL-1 (F9) or CICL-207 (F50) lipid compositions. The bars represent the mean of one to three mice with the symbols indicating results from mice engrafted with human CD34⁺ HSPC from three different donors (triangles, squares and circles).

FIGS. 12A-12H depict the levels of various liver enzymes, acute phase proteins, and cytokines in rats administered the F50 LNP. FIGS. 12A-12B plot mean liver enzyme levels in rat serum at 24 hours after dosage of F50 LNP composition for aspartate aminotransferase (AST) (FIG. 12A) and alanine amino transferase (ALT) (FIG. 12B). FIGS. 12C-12E depict the levels of three acute phase proteins in rat plasma after administration of the indicated doses of the F50 LNP at 6 and 24 hours after administration, specifically α1-acid glycoprotein (FIG. 12C), α2-macroglobulin (FIG. 12D), and haptoglobin (FIG. 12E). FIGS. 12F-12H show the levels of three pro-inflammatory cytokines in rat plasma after administration of the indicated doses of the F50 LNP at 6 and 24 hours after administration, specifically interleukin-6 (FIG. 12F), interleukin-1β (FIG. 12G), tumor necrosis factor-α (FIG. 12H).

### DETAILED DESCRIPTION

This disclosure provides constrained ionizable cationic lipids, referred to as ionizable cationic lipids throughout, methods for synthesizing them, as well as intermediates useful in synthesis of these lipids and methods of synthesizing the intermediates. This disclosure provides ionizable cationic lipids as a component of lipid nanoparticles (LNPs) that can be used for delivering a biologically active payload (e.g., nucleic acid molecules encoding a therapeutic agent) into cells in vivo or ex vivo. LNP compositions are also disclosed herein, including LNPs comprising a functionalized PEG-lipid to enable conjugation of a binding moiety to generate targeted LNPs (tLNPs), that is LNPs containing a binding moiety that directs the tLNP to a desired tissue or cell type (e.g., immune cells such as T cells or stem cells such as hematopoietic stem cells (HSCs)). Also disclosed herein are methods of delivering a nucleic acid into a cell comprising contacting the cell with an LNP or tLNP of this disclosure. The LNP and tLNP of this disclosure can be used for in vivo, ex vivo, or extracorporeal transfection. Also disclosed herein are methods for preparing LNPs and tLNPs comprising the ionizable cationic lipids as described herein.

Prior to setting forth this disclosure in more detail, it can be helpful to provide abbreviations and definitions of certain terms to be used herein. Additional abbreviations are set forth throughout this disclosure.

### ABBREVIATIONS

Abbreviations used herein include:
BOC₂O—Di-tert-butyl dicarbonate
CDCl₃—Deuterochloroform
CDI—carbonyl diimidazole
CH₃CN—Acetonitrile
CHOL—Cholesterol
DMAP—4-Dimethylaminopyridine
DMG—Dimyristoyl glycerol
DSG—Distearoyl glycerol
DSPC—Distearoylphosphatidylcholine
DSPE—Distearoylphosphatidylethanolamine
EDC-HCl—1-Ethyl-3-(3′-dimethylaminopropyl)carbodiimide HCl
EtOAc—Ethyl acetate
EtOH—Ethanol
Et₂O—Diethyl ether
Et₃N—Triethylamine
MAL—Maleimide
Me₃N—Trimethylamine
MeOH—Methanol
MeOTf—methyl trifluoromethanesulfonate
Pd/C—Palladium on carbon
PEG—Polyethylene glycol
PhCH₃—Toluene
TFA—Trifluoroacetic acid
THF—Tetrahydrofuran

While this disclosure is capable of being embodied in various forms, the description below of several embodiments is made with the understanding that the disclosure is to be considered as an exemplification of the innovations disclosed herein and is not intended to limit the disclosure to the specific embodiments illustrated.

Headings are provided for convenience only and are not to be construed to limit the embodiments in any manner. Embodiments illustrated under any heading can be combined with embodiments illustrated under any other heading.

To the extent any materials incorporated herein by reference conflict with this disclosure, this disclosure controls.

### Definitions

Prior to setting forth this disclosure in more detail, provided are definitions of certain terms to be used herein. Additional definitions are set forth throughout this disclosure.

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives.

The term “about” as used herein in the context of a number refers to a range centered on that number and spanning 10% less than that number and 10% more than that number. The term “about” used in the context of a range refers to an extended range spanning 10% less than that of the lowest number listed in the range and 10% more than the greatest number listed in the range.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

Unless the context requires otherwise, throughout this specification and claims, the word “comprise” and variations thereof, such as, “comprises” and “comprising” are to be construed in an open, inclusive sense, that is, as “including, but not limited to.” As used herein, the terms “include” and “comprise” are used synonymously.

The phrase “at least one of” when followed by a list of items or elements refers to an open-ended set of one or more of the elements in the list, which can, but does not necessarily, include more than one of the elements.

“Derivative,” as used herein, refers to a chemically or biologically modified version of a compound that is structurally similar to a parent compound and (actually or theoretically) derivable from that parent compound. Generally, a “derivative” differs from an “analogue” in that a parent compound may be the starting material to generate a “derivative,” whereas the parent compound is not necessarily used as the starting material to generate an “analogue.” A derivative may have different chemical or physical properties than the parent compound. For example, a derivative may be more hydrophilic or hydrophobic, or it may have altered reactivity as compared to the parent compound. Although a derivative can be obtained by physical (for example, biological or chemical) modification of the parent compound, a derivative can also be conceptually derived, for example, as when a protein sequence is designed based on one or more known sequences, an encoding nucleic acid is constructed, and the derived protein obtained by expression of the encoding nucleic acid.

As used herein “expansion” refers to proliferation of cells increasing their number. Activating agents can be used to stimulate proliferation (among other metabolic changes) but can also result in activation induced death upon initial exposure so that there is no immediate expansion. For T cells treated in vitro with activating agents such as IL-2 or CD3/CD28 activators, doubling time can be about 24 hours (which is fairly typical of mammalian cells in vitro generally); in vivo doubling time can be substantially shorter, depending on the presence and type of stimulation. Accordingly, during a limited time of extracorporeal manipulation, even when activating agents are used, such protocols will be effectively expansion-less.

As used herein an “exogenous protein” refers to a synthetic, recombinant, or other peptide or protein that is not produced by a wild-type cell of that type or is expressed at a lower level in a wild-type cell than in a cell containing the exogenous polypeptide. In some embodiments, an exogenous peptide is a peptide or protein encoded by a nucleic acid that was introduced into the cell, which nucleic acid is optionally not retained by the cell. As used herein “peptide” refers to a chain of amino acids less than 50 amino acids in length, while “protein” and “polypeptide refer to a chain of amino acids at least 50 amino acids in length.

As used herein “extracorporeal” is used in reference to cells, such as peripheral blood or bone marrow cells, harvested or extracted from the body and the manipulation or modification of those cells prior to their intended return (reinfusion). Manipulation and modification of cells generally relates to cell separation and washing procedures and exposure to activation agents (e.g., biological response modifiers (BRMs)) and transfection agents (e.g., LNPs, tLNPs), over a time interval of several hours, for example, less than 6 hours, less than 5 hours, less than 4 hours, less than 3 hours, less than 2 hours, or less than 1 hour; and in space to a single institution. Extracorporeal is used in contradistinction to ex vivo which, as used herein, includes more extensive manipulation including extended periods of cell culture and expansion, and/or refrigerated or cryogenic storage or shipment, over several days or longer.

As used herein “transfection” or “transfecting” refers to the introduction of nucleic acids into cells by non-viral methods. Transfection can be mediated by calcium phosphate, cationic polymers, magnetic beads, electroporation, and lipid-based reagents. In preferred embodiments disclosed herein transfection is mediated by solid lipid nanoparticles (LNP) including targeted LNP (tLNP) (which can also be used to deliver non-nucleic acid payloads into cells). The term transfection is used in distinction to transduction—transfer of genetic material from cell to cell or virus to cell—and transformation—the uptake of extracellular genetic material by the natural processes of a cell. As used herein, phrases such as “delivering a nucleic acid into a cell” are synonymous with transfection.

“Reprogramming,” as used herein with respect to immune cells, refers to changing the functionality of an immune cell with respect to antigenic specificity by causing expression of an exogenous T cell receptor (TCR), a chimeric antigen receptor (CAR), or an immune cell engager (“reprogramming agents”). Generally, T lymphocytes and natural killer (NK) cells could be reprogrammed with a TCR, a CAR, or an immune cell engager while only a CAR or an immune cell engager would be used in reprogramming monocytes. As used herein with respect to stem cells, for example hematopoietic stem cells (HSC) or mesenchymal stem cells (MSC), “reprogramming” refers to correction or amelioration of a genetic defect (for example, a hemoglobinopathy) so that the modified or corrected gene and gene product are the reprogramming agents. Reprogramming can be transient or durable depending on the nature of the engineering agent.

“Engineering agent,” as used herein, refers to agents that confer the expression of a reprogramming agent by an immune cell, particularly a non-B lymphocyte or monocyte. Engineering agents can include nucleic acids, including mRNA, that encode the reprogramming agent. Engineering agents can also include nucleic acids that are or encode components of gene editing systems such as RNA-guided nucleases, guide RNA, and nucleic acid templates for knocking-in a reprogramming agent or knocking-out an endogenous antigen receptor. Gene editing systems comprise base-editors, prime-editors or gene-writers. RNA-guided nucleases include CRISPR nucleases such as Cas9, Cas12, Cas13, Cas3, CasMINI, Cas7-11, and CasX. For transient expression of a reprogramming agent, such as a CAR, an mRNA encoding the reprogramming agent can be used as the engineering agent. For durable expression of the reprogramming agent, such as an exogenous, modified, or corrected gene (and its gene product), the engineering agent can comprise mRNA-encoded RNA-directed nucleases, guide RNAs, nucleic acid templates and other components of gene/genome editing systems.

Examples of gene editing components that are encoded by a nucleic acid molecule include an mRNA encoding an RNA-guided nuclease, a gene or base editing protein, a prime editing protein, a Gene Writer protein (e.g., a modified or modularized non-long terminal repeat (LTR) retrotransposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-MLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Other components include a guide RNA (gRNA), a single guide RNA (sgRNA), a prime editing guide RNA (pegRNA), a clustered regularly interspaced short palindromic repeat (CRISPR) RNA (crRNA), a trans-activating clustered regularly interspaced short palindromic repeat (CRISPR) RNA (tracrRNA), or a DNA molecule to be inserted or serve as a template for double-strand break (DSB) repair at a specific genomic locus. Genome-, gene-, and base-editing technology are reviewed in Anzalone et al., Nature Biotechnology 38:824-844, 2020, Sakuma, Gene and Genome Editing 3-4:100017, 2022, and Zhou et al., Med Comm 3(3):e155, 2022, each of which is incorporated by reference for all that they teach about the components and uses of this technology to the extent that it does not conflict with the present disclosure.

“Conditioning agent,” as used herein, refers to a biological response modifier (BRM) that enhances the efficiency of engineering an immune cell, expands the number of immune cells available to be engineered or the number of engineered cells in a target tissue (for example, a tumor, fibrotic tissue, or tissue undergoing autoimmune attack), promotes activity of the engineered cell in a target tissue, or broadens the range of operative mechanisms contributing to a therapeutic immune reaction. A conditioning agent may be provided by delivering an encoding nucleic acid in a tLNP. Exemplary BRMs include cytokines, such as IL-7, IL-15, or IL-18.

“Immune cell,” as used herein, can refer to any cell of the immune system. However, particular aspects can exclude polymorphonuclear leukocytes and/or B cells, or be limited to non-B lymphocytes such as T cells and/or NK cells, or to monocytes such as dendritic cells and/or macrophages in their various forms.

As used herein, “lipid nanoparticle” (LNP) means a solid particle, as distinct from a liposome having an aqueous lumen. The core of an LNP, like the lumen of a liposome, is surrounded by a layer of lipid that can be, but is not necessarily, a continuous lipid monolayer, a bilayer, or multi-layer having three or more lipid layers.

As used herein, a “binding moiety” or “targeting moiety” refers to a protein, polypeptide, oligopeptide, peptide, carbohydrate, nucleic acid, or combination thereof that is capable of specifically binding to a target or multiple targets. A binding domain includes any naturally occurring, synthetic, semi-synthetic, or recombinantly produced binding partner for a biological molecule or another target of interest. Exemplary binding moieties of this disclosure include an antibody, a Fab′, F(ab′)₂, Fab, Fv, rIgG, scFv, hcAbs (heavy chain antibodies), a single domain antibody, VHH, VNAR, sdAbs, nanobody, receptor ectodomains or ligand-binding portions thereof, or ligands (e.g., cytokines, chemokines). A “Fab” (fragment antigen binding) is the part of an antibody that binds to antigens and includes the variable region and CH1 of the heavy chain linked to the light chain via an inter-chain disulfide bond. In other embodiments, a binding moiety comprises a ligand-binding domain of a receptor or a receptor ligand. In some embodiments, a binding moiety can have more than one specificity including, for example, bispecific or multispecific binders. A variety of assays are known for identifying binding moieties of this disclosure that specifically bind a particular target, including Western blot, ELISA, and Biacore® analysis. A binding moiety, such as a binding moiety comprising immunoglobulin light and heavy chain variable domains (e.g., scFv), can be incorporated into a variety of protein scaffolds or structures as described herein, such as an antibody or an antigen binding fragment thereof, a scFv-Fc fusion protein, or a fusion protein comprising two or more of such immunoglobulin binding domains.

As used herein, “antibody” refers to a protein comprising an immunoglobulin domain having hypervariable regions determining the specificity with which the antibody binds antigen; so-called complementarity determining regions (CDRs). The term antibody can thus refer to intact or whole antibodies as well as antibody fragments and constructs comprising an antigen binding portion of a whole antibody. While the canonical natural antibody has a pair of heavy and light chains, camelids (camels, alpacas, llamas, etc.) produce antibodies with both the canonical structure and antibodies comprising only heavy chains. The variable region of the camelid heavy chain only antibody has a distinct structure with a lengthened CDR3 referred to as VHH or, when produced as a fragment, a nanobody. Antigen binding fragments and constructs of antibodies include F(ab)₂, F(ab), minibodies, Fv, single-chain Fv (scFv), diabodies, and VH. Such elements can be combined to produce bi- and multi-specific reagents, such as Bispecific T-Cell Engagers (BiTEs). The term “monoclonal antibody” arose out of hybridoma technology but is now used to refer to any singular molecular species of antibody regardless of how it was originated or produced. Antibodies can be obtained through immunization, selection from a naïve or immunized library (for example, by phage display), alteration of an isolated antibody-encoding sequence, or any combination thereof. Numerous antibodies that could be used as binding moieties are known in the art. An excellent source of information about antibodies for an International Non-proprietary Name (INN) has been proposed or recommended, including sequence information, is Wilkinson & Hale, 2022, MAbs 14(1):2123299, including its Supplementary Tables, which is incorporated by reference herein for all that it teaches about individual antibodies and the various antibody formats that can be constructed. U.S. Pat. No. 11,326,182 and especially its Table 9 entitled “Cancer, Inflammation and Immune System Antibodies,” is a source of sequence and other information for a wide range of antibodies including many that do not have an INN and is incorporated herein by reference for all that it teaches about individual antibodies.

An antibody or other binding moiety (or a fusion protein thereof) “specifically binds” a target if it binds the target with an affinity or Ka (i.e., an equilibrium association constant of a particular binding interaction with units of 1/M) equal to or greater than 10⁵ M⁻¹, while not significantly binding other components present in a test sample. Binding domains (or fusion proteins thereof) can be classified as “high affinity” binding domains (or fusion proteins thereof) and “low affinity” binding domains (or fusion proteins thereof). “High affinity” binding domains refer to those binding domains with a Ka of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Ka of up to 10⁸ M⁻¹, up to 107 M⁻¹, up to 10⁶ M⁻¹, up to 10⁵ M⁻¹. Alternatively, affinity can be defined as an equilibrium dissociation constant (Kd) of a particular binding interaction with units of M (e.g., 10⁻⁵ M to 10⁻¹³ M). Affinities of binding domain polypeptides and fusion proteins according to this disclosure can be readily determined using conventional techniques (see, e.g., Scatchard et al., 1949, Ann. N.Y. Acad. Sci. 51:660; and U.S. Pat. Nos. 5,283,173, 5,468,614, or the equivalent).

As used herein, “payload” refers to a negatively charged biologically active agent that can interact with cationic lipids, such as the ionizable cationic lipids of this disclosure, to become encapsulated within lipid nanoparticles comprising the cationic lipid. The negatively charged, biologically active agent can be a small organic molecule, or a macromolecule such as a nucleic acid, a carbohydrate, or a peptide or polypeptide. In many embodiments, the payload can be one or more nucleic acid molecules, RNA or DNA, including mRNA and guide RNA (gRNA) molecules.

As used herein, “biologically active agent” refers to any substance, or a component of a combination of substances, that affects a metabolic or physiologic response in a living organism or cultured cells thereof.

As used herein, “therapeutic agent” is a substance the biological activity of which can potentially cure, ameliorate, stabilize, prevent, or otherwise beneficially impact a disease, pathological condition, or other disorder.

For simplicity, chemical moieties are defined and referred to throughout primarily as univalent chemical moieties (e.g., alkyl, aryl, and the like). Nevertheless, such terms can also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S).

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups.

In some embodiments, the hydrocarbon chain is unsubstituted. In other embodiments, one or more hydrogens of the alkyl or alkenyl group can be substituted with the same or different substituents.

Alkynoic refers to a carboxylic acid moiety comprising one or more carbon-carbon triple bonds. In some embodiments, hydrogens are unsubstituted. In other embodiments, one or more hydrogens of the alkynoic group can be substituted with the same or different substituents.

Amide refers to a carboxylic acid derivative comprising a carbonyl group of a carboxylic acid bonded to an amine moiety.

Ester refers to a carboxylic acid derivative comprising a carbonyl group bond to an alkyloxy group to form an ester bond —C(═O)—O—.

Head group refers to the hydrophilic or polar portion of a lipid.

Sterol refers to a subgroup of steroids that contain at least one hydroxyl (OH) group. Examples of sterols include, without limitation, cholesterol, ergosterol, s-sitosterol, stigmasterol, stigmastanol, 20-hydroxycholesterol, 22-hydroxycholesterol, and the like.

As standard, the bond represented as a solid wedge extends above the plane and the bond represented as a dashed wedge extends below the plane when depicting absolute stereochemistry, throughout.

As compared to similar molecules in which two branching tails are unconnected to each other, in the constrained ionizable lipids described herein, a ring comprising a central branchpoint nitrogen atom connects the two branching tail groups to each other, reducing their freedom of movement. As used herein, “constrained” refers to this limited freedom of movement of the two tail groups extending from a ring comprising a nitrogen atom.

### Ionizable Cationic Lipids

In designing families of chemical compounds, it is common to have a constant or near-constant core and vary the pendant groups attached to it. A different approach has been taken with the ionizable cationic lipids disclosed herein. A family of ionizable cationic lipids within the general structure of Formula 1b are provided where R represents hydrophobic tails of the lipid, n represents an integer from 0 to 4, and X constitutes the polar headgroup comprising an ionizable amine.

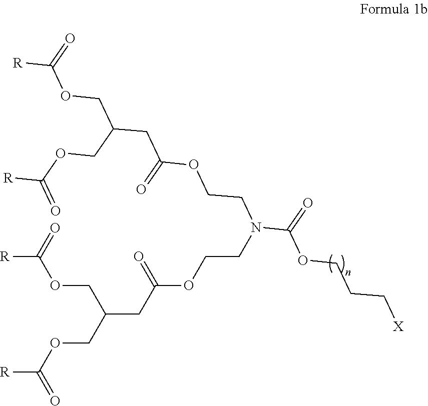

Such lipids are useful as a component of lipid nanoparticles (LNPs) for delivery of nucleic acids into cells. It is convenient to draw the structure of the lipids as shown here both for compactness and because it illustrates the conical shape that is understood promotes endosomal escape of the contents of LNPs comprising such lipids. However, this is only one possible conformation the lipids can adopt. Entropy will favor adoption of multiple conformations.

The orientation of the three groups extending from the central branchpoint nitrogen atom in Formula 1b can be constrained by forming a ring comprising that nitrogen atom and to which the two branching tail groups are attached, for example, Formula 1c:

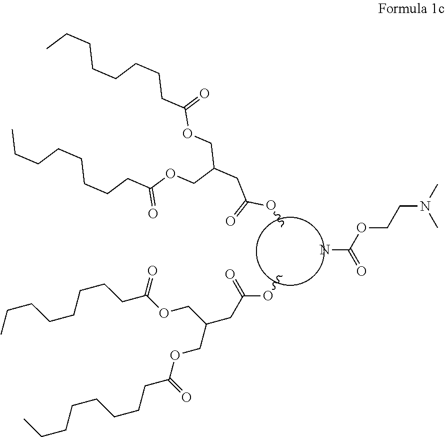

By introducing this constraint, the number of conformations the lipids can adopt is reduced. This can increase the physical stability of LNPs or tLNPs incorporating such constrained lipids as compared to LNPs or tLNPs incorporating similar lipids lacking a constraining ring.

The size of the ring can be varied as can the positioning of the tail groups' attachment positions, whether the tail groups are directly attached to the ring or if additional carbons are interposed, whether the tail groups are cis or trans so that the substituents are axial or equatorial, or whether the tail groups are attached symmetrically or asymmetrically on the ring. However, these variations should preserve the high degree of biodegradability believed to be conferred by these branched tail groups, and the head group can be altered to attain a favorable c-pKa and c Log D.

In certain aspects, the constrained ionizable cationic lipids of this disclosure have a structure of formula M2:

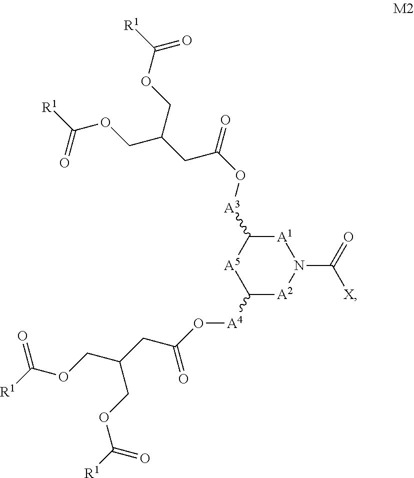

wherein X is

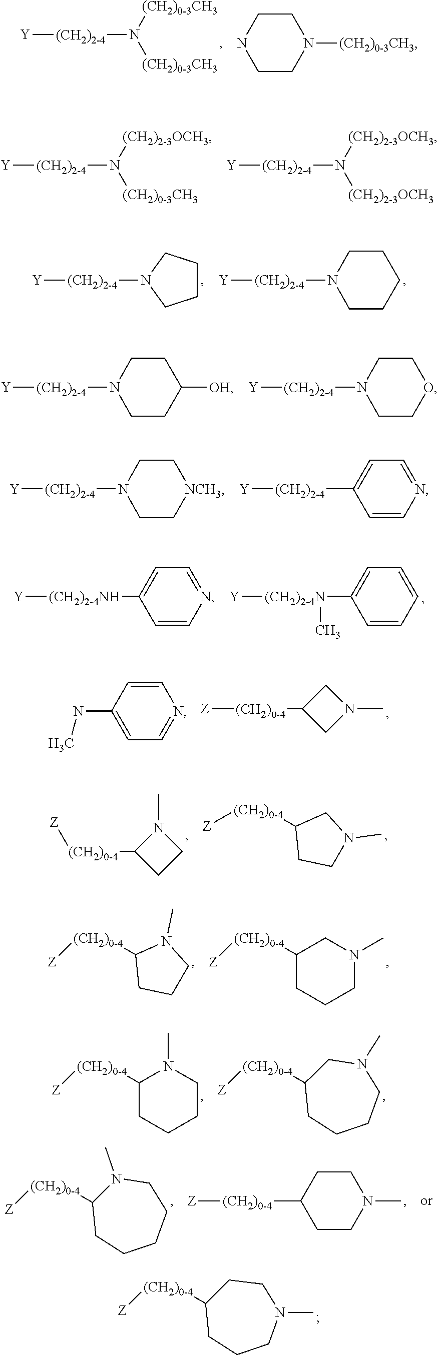

Y is O, S, NH, or NCH₃;
Z is O, NH, or NCH₃;
each R¹ is independently selected from C₇-C₁₁ alkyl or C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
a wavy bond indicates that any relative or absolute stereo-configuration of the corresponding ring atom, or a mixture of stereo-configurations, can be assumed.

In various embodiments, A¹ through A⁴ are selected so that there are only two main chain atoms between the ring nitrogen and each nearest ester oxygen in the nearest tail group.

In certain embodiments, A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

In certain embodiments, A¹ is (CH₂)₀, A² is (CH₂)₁, A³ is (CH₂)₁, A⁴ is (CH₂)₀, and A⁵ is (CH₂)₁.

In certain embodiments, A¹ is (CH₂)₁, A² is (CH₂)₁, A³ is (CH₂)₀, A⁴ is (CH₂)₀, and A⁵ is (CH₂)₀.

In certain embodiments, A¹ is (CH₂)₁, A² is (CH₂)₁, A³ is (CH₂)₀, A⁴ is (CH₂)₀, and A⁵ is (CH₂)₁.

In certain embodiments, A¹ is (CH₂)₁, A² is (CH₂)₁, A³ is (CH₂)₀, A⁴ is (CH₂)₀, and A⁵ is (CH₂)₂ or CH═CH.

Ionizable cationic lipids of this disclosure have a branched structure to give the lipid a conical rather than cylindrical three-dimensional shape and such structure helps promote endosomolytic activity. The greater the endosomolytic activity, the more efficient release of the nucleotide cargo.

In certain embodiments, the ionizable cationic lipid is substantially enantiomerically pure (e.g., at least about 80%, at least about 85%, at least about 90%, at least about 95%, at least about 96%, at least about 97%, at least about 98%, or at least about 99% pure). In certain embodiments, the ionizable cationic lipid is a racemic mixture. In certain embodiments, the ionizable cationic lipid is a mixture of two or more stereoisomers. In certain embodiments, at least two of the two or more stereoisomers are diastereomers. In certain embodiments, at least two of the two or more stereoisomers are enantiomers.

Ionizable cationic lipids as described herein, can be useful as a component of lipid nanoparticles for delivering nucleic acids, including DNA, mRNA, or siRNA into cells. The ionizable cationic lipids can have a c-pKa (calculated pKa) in the range of from about 6, 7, or 8 to about 9, 10, or 11. For example, in various embodiments as described herein, the ionizable cationic lipids have a c-pKa ranging from about 6 to about 10, about 7 to about 10, about 8 to about 10, about 8 to about 9, 6 to 10, 7 to 10, 8 to 10, or 8 to 9. In certain embodiments, the ionizable cationic lipids have a c-pKa ranging from about 8.2 to about 9.0 or from 8.2 to 9.0. In certain embodiments, the ionizable cationic lipids have a c-pKa ranging from about 8.4 to about 8.7 or from 8.4 to 8.7. The ionizable cationic lipids as described herein can have c Log D ranging from about 9 to about 18, for example, ranging from about 10 to about 18, or about 10 to about 16, to about 10 to about 14, or about 11 to about 18, or about 11 to about 15, or about 11 to about 14. The ionizable cationic lipids as described herein can have c Log D ranging from 9 to 18, for example, ranging from 10 to 18, or 10 to 16, to 10 to 14, or 11 to 18, or 11 to 15, or 11 to 14. In certain embodiments, the ionizable cationic lipids have a c Log D ranging from about 13.6 to about 14.4 or from 13.6 to 14.4. In certain embodiments, the ionizable cationic lipids as described herein can have a c-pKa ranging from about 8 to about 11 or from 8 to 11 and a c Log D ranging from about 9 to about 18 or from 9 to 18. For example, in certain embodiments, the ionizable cationic lipids have a c-pKa ranging from about 8.4 to about 8.7 or from 8.4 to 8.7 and c Log D ranging from about 13.6 to about 14.4 or from 13.6 to 14.4. These ranges can lead to a measured pKa in the LNP ranging from about 6 to about 7 or from 6 to 7, which facilitates ionization in an endosome after delivery into a cell.

In some embodiments, somewhat greater basicity can be desirable and can be obtained from ionizable cationic lipids with c-pKa and c Log D in the stated ranges. In some embodiments c Log D is about 9, about 10, about 11, about 12, about 13, about 14, about 15, about 16, about 17, about 18, or in a range bound by any pair of these values. Lipid design also accounts for potential biodegradability pathways of target lipids, utilizing esterases in plasma, liver and other tissues. Another consideration in lipid design is the fate of fragments of ionizable lipids resulting after esterase cleavage(s). Preferably, the resulting fragments are rapidly cleared from the body without the intervention of hepatic oxidative metabolism.

As used herein, c Log D is a calculated measure of lipophilicity that accounts for the state of ionization of the molecule at a particular pH, which is a predictor of partitioning of the lipid between water and octanol as a function of pH. More specifically, c Log D is calculated at a specified pH based on c Log P and c-pKa. (Log P is the partition coefficient of a molecule between aqueous (e.g., water) and lipophilic phases (e.g., octanol)). Numerous software packages are available to provide values of c Log D. When higher basicity of the ionizable lipid is desired, it should be balanced by greater lipophilicity as represented by a higher c Log D value. Balance of basicity and lipophilicity is needed for optimal functioning of the LNP for both stability of the particle and release of the biologically active payload (e.g., one or more species of nucleic acid molecule that can encode a therapeutic agent) upon uptake by a cell. Accordingly, as R¹ increases from C6-C10, the overall lipophilicity of the ionizable cationic lipid will increase, as represented by c Log D. This can be balanced by alterations in X, which result in higher c-pKa based on the basicity of the head group. Each of the ionizable cationic lipid species described herein have a c Log D and c-pKa value within the desired range(s), as described herein. Specific c Log D and c-pKa values have been calculated using ACD Labs Structure Designer v 12.0 for ionic cationic lipids of the disclosure. c Log P was calculated using ACD Labs Version B; c Log D was calculated at pH 7.4. Table 1 shows c Log D and c-pKa for CICL-207, CICL-215, CICL-216, CICL-217, CICL-218, CICL-219, CICL-220, CICL-221, CICL-222, CICL-223, CICL-224, and CICL-225.

TABLE 1
cLogD and c-pKa for CICL-207, CICL-215, CICL-216,
CICL-217, CICL-218, CICL-219, CICL-220, CICL-221,
CICL-222, CICL-223, CICL-224, CICL-225, CICL-238,
CICL-239, CICL-242, CICL-243, CICL-244, CICL-245,
CICL-246, CICL-247, CICL-248, and CICL-249.

| | c-LogD | |
|---|---|---|
| Lipid | (pH 7.4) | c-pKa |
| CICL-207 | 13.75 | 8.42 |
| CICL-215 | 13.67 | 8.42 |
| CICL-216 | 13.67 | 8.42 |
| CICL-217 | 14.34 | 8.62 |
| CICL-218 | 13.71 | 8.49 |
| CICL-219 | 13.71 | 8.49 |
| CICL-220 | 13.80 | 8.50 |
| CICL-221 | 13.67 | 8.42 |
| CICL-222 | 13.67 | 8.42 |
| CICL-223 | 13.75 | 8.42 |
| CICL-224 | 13.75 | 8.42 |
| CICL-225 | 13.75 | 8.42 |
| CICL-238 | 13.80 | 8.50 |
| CICL-239 | 14.77 | 7.16 |
| CICL-242 | 13.95 | 8.42 |
| CICL-243 | 13.95 | 8.42 |
| CICL-244 | 14.50 | 8.50 |
| CICL-245 | 14.50 | 8.50 |
| CICL-246 | 14.95 | 8.49 |
| CICL-247 | 14.95 | 8.49 |
| CICL-248 | 14.94 | 8.50 |
| CICL-249 | 14.94 | 8.50 |

Different constituents for X, Y, and R¹ allow for tuning of c Log D and c-pKa to achieve a desired value of measured pKa within a LNP or a tLNP. For example, to make a lipid with a CH₃ head group of

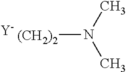

less basic, the following head groups could be used instead:

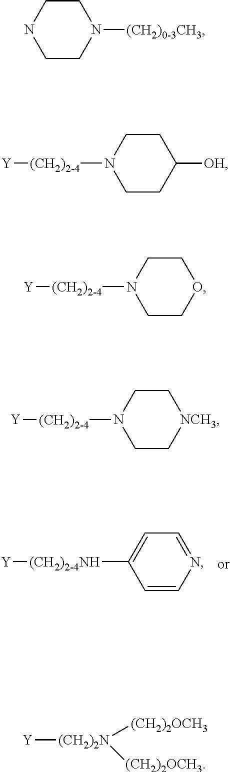

Conversely, to make a lipid with a head group of

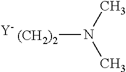

more basic, the following head groups could be used instead:

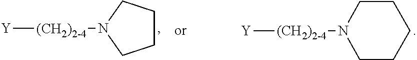

The addition of CH₂ groups in X will tend to increase basicity of the lipid which in turn will tend to increase measured pKa. The addition of CH₂ groups in R¹ will tend to increase the lipophilicity (c Log D) of the lipid which in turn will tend to decrease measured pKa of the LNP or tLNP.

The use of further head groups to modify calculated pKa and target a desired measured pKa range is set out in Example 65.

In some embodiments as described herein, X is

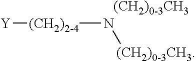

For example, in some embodiments X is

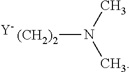

In some embodiments as described herein, X is

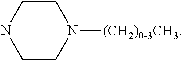

In some embodiments X is

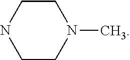

In some embodiments as described herein, X is

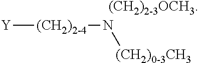

In some embodiments as described herein, X is

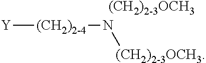

In some embodiments as described herein, X is

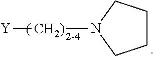

In some embodiments as described herein, X is

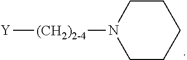

In some embodiments as described herein, X is

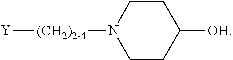

In some embodiments as described herein, X is

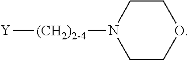

In some embodiments as described herein, X is

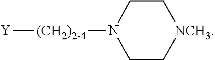

In some embodiments as described herein, X is

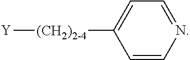

In some embodiments as described herein, X is

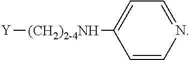

In some embodiments as described herein, X is

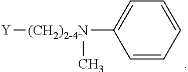

In some embodiments as described herein, X is

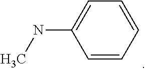

In some embodiments as described herein, X is

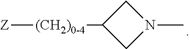

In some embodiments as described herein, X is

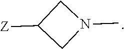

In some embodiments as described herein, X is

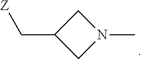

In some embodiments as described herein, X is

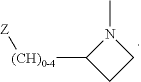

In some embodiments as described herein, X is

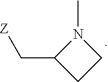

In some embodiments as described herein, X is

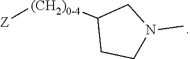

In some embodiments as described herein, X is

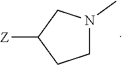

In some embodiments as described herein, X is

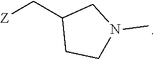

In some embodiments as described herein, X is

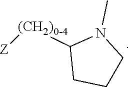

In some embodiments as described herein, X is

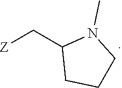

In some embodiments as described herein, X is

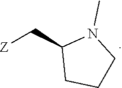

In some embodiments as described herein, X is

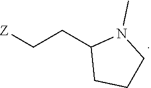

In some embodiments as described herein, X is

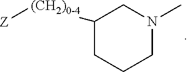

In some embodiments as described herein, X is

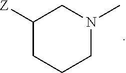

In some embodiments as described herein, X is

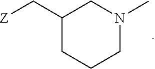

In some embodiments as described herein, X is

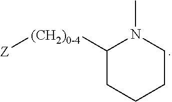

In some embodiments as described herein, X is

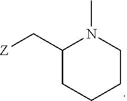

In some embodiments as described herein, X is

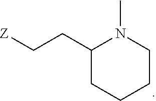

In some embodiments as described herein, X is

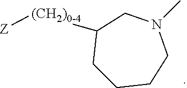

In some embodiments as described herein, X is

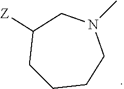

In some embodiments as described herein, X is

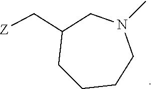

In some embodiments as described herein, X is

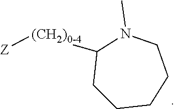

In some embodiments as described herein, X is

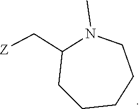

In some embodiments as described herein, X is

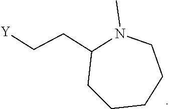

In some embodiments as described herein, X is

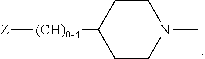

In some embodiments as described herein, X is

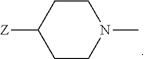

In some embodiments as described herein, X is

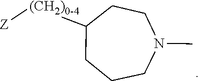

In some embodiments as described herein, X is

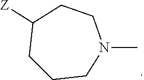

As described above, Y can be selected from O, S, NH, or NCH₃. In some embodiments, Y is O. In some other embodiments, Y is S.

In some embodiments, X is

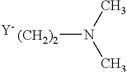

and Y is O. In some embodiments X is

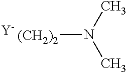

### and Y is S.

As described above, Z can be selected from O, NH, or NCH₃. In some embodiments, Z is O.

In some embodiments as described herein, X is

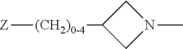

and Z is O. In some embodiments as described herein, X is

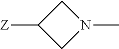

and Z is O. In some embodiments as described herein, X is

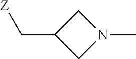

### and Z is O.

In some embodiments, X is

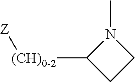

and Z is O. In some embodiments as described herein, X is

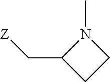

### and Z is O.

In some embodiments as described herein, X is

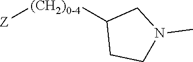

and Z is O. In some embodiments as described herein, X is

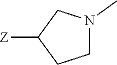

and Z is O. In some embodiments as described herein, X is

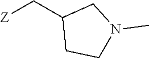

### and Z is O.

In some embodiments as described herein, X is

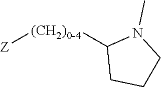

and Z is O. In some embodiments as described herein, X is

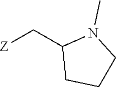

and Z is O. In some embodiments as described herein, X is

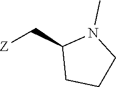

and Z is O. In some embodiments as described herein, X is

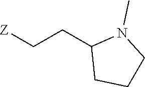

### and Z is O.

In some embodiments as described herein, X is

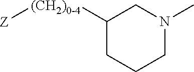

and Z is O. In some embodiments as described herein, X is

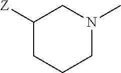

and Z is O. In some embodiments as described herein, X is

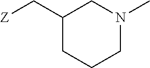

### and Z is O.

In some embodiments as described herein, X is

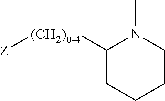

and Z is O. In some embodiments as described herein, X is

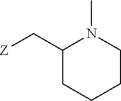

and Z is O. In some embodiments as described herein, X is

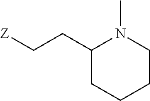

### and Z is O.

In some embodiments as described herein, X is

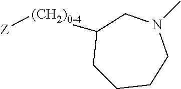

and Z is O. In some embodiments as described herein, X is

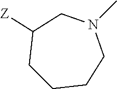

and Z is O. In some embodiments as described herein, X is

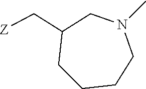

### and Z is O.

In some embodiments as described herein, X is

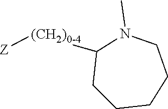

and Z is O. In some embodiments as described herein, X is

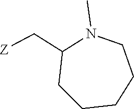

and Y is O. In some embodiments as described herein, X is

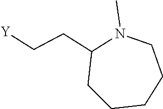

### and Z is O.

In some embodiments a described herein, X is

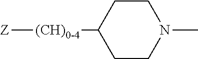

and Z is O. In some embodiments as described herein, X is

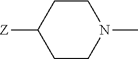

### and Z is O.

In some embodiments as described herein, X is

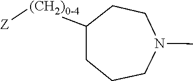

and Z is O. In some embodiments as described herein, X is

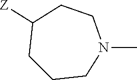

### and Z is O.

As described above, each R¹ is independently selected from C₇-C₁₁ alkyl or C₇-C₁₀ alkene. In some embodiments, each R¹ is independently selected from C₇-C₁₁ alkyl, e.g., C₇-C₁₀ alkyl, or C₇-C₉ alkyl. In certain embodiments, each R¹ is independently selected from a linear C₇-C₁₁ alkyl, e.g., a linear C₇-C₁₀ alkyl, or a linear C₇-C₉ alkyl. In some embodiments as described herein, each R¹ is independently selected from (CH₂)₆₋₈CH₃. In some of these and other embodiments, R1 is (CH₂)₇CH₃. In some embodiments, each R¹ is independently selected from a linear C₇-C₁₁ alkenyl, e.g., a linear C₇-C₁₀ alkenyl, or a linear C₇-C₉ alkenyl. For example, in some embodiments, each R¹ is a linear C₈ alkenyl. In certain other embodiments, each R¹ is independently selected from a branched C₇-C₁₁ alkyl, e.g., C₇-C₁₀ alkyl, or C₇₋-C₉ alkyl. For example, in some embodiments, each R¹ is a branched C₈ alkyl. In certain embodiments, each R¹ is independently selected from a branched C₇-C₁₁ alkenyl, e.g., C₇-C₁₀ alkenyl, or C₇-C₉ alkenyl. For example, in some embodiments, each R¹ is a branched C₈ alkenyl. In some embodiments, wherein R¹ is a branched alkyl or alkenyl, the branch point is positioned so that ester carbonyls are not in an a position relative to the branch point, for example they are in a β position relative to the branch point.

In certain embodiments as described herein, each R¹ is the same. In certain embodiments, each R¹ nearest a common branch point is the same, but those nearest a first common branch point differ from those nearest a second common branch point. In certain embodiments, each R¹ nearest a common branch point is different but the pair of R's nearest a first common branch point is the same the pair nearest a second common branch point.

As described above, in some embodiments as described herein, the ionizable cationic lipid has a structure of formula M2, wherein A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂. For example, in some embodiments as described herein, A¹ is (CH₂)₀, A² is (CH₂)₀, A³ is (CH₂)₁, A⁴ is (CH₂)₁, and A⁵ is (CH₂)₁. In some embodiments as described herein, the ionizable cationic lipid has the structure:

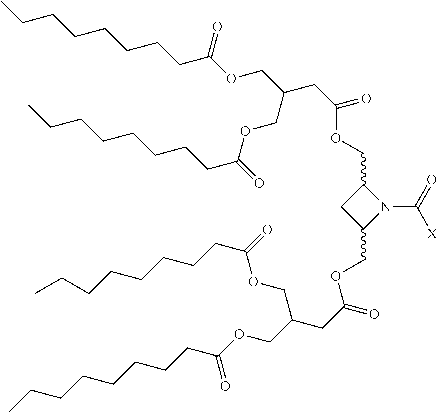

wherein X is as described herein and wavy bonds indicate that any relative or absolute stereo-configuration of the corresponding ring atom can be assumed.

In certain embodiments of formula M2 as described herein, A¹ is (CH₂)₀, A² is (CH₂)₀, A³ is (CH₂)₁, A⁴ is (CH₂)₁, and A⁵ is (CH₂)₂. For example, in some embodiments, the ionizable cationic lipid has the structure:

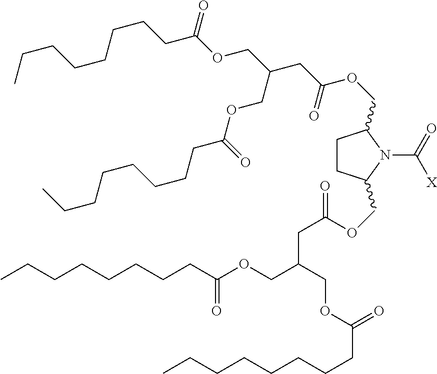

wherein X is as described herein and wavy bonds indicate that any relative or absolute stereo-configuration of the corresponding ring atom can be assumed.

In certain embodiments of formula M2 as described herein, A¹ is (CH₂)₀, A² is (CH₂)₀, A³ is (CH₂)₁, A⁴ is (CH₂)₁, and A⁵ is (CH₂)₃. For example, in some embodiments, the ionizable cationic lipid has the structure:

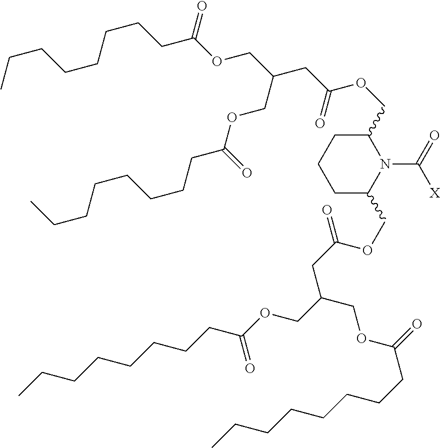

wherein X is as described herein and wavy bonds indicate that any relative or absolute stereo-configuration of the corresponding ring atom can be assumed.

In certain embodiments of formula M2 as described herein, A¹ is (CH₂)₀, A² is (CH₂)₀, A³ is (CH₂)₁, A⁴ is (CH₂)₁, and A⁵ is (CH₂)₄. For example, in some embodiments, the ionizable cationic lipid has the structure:

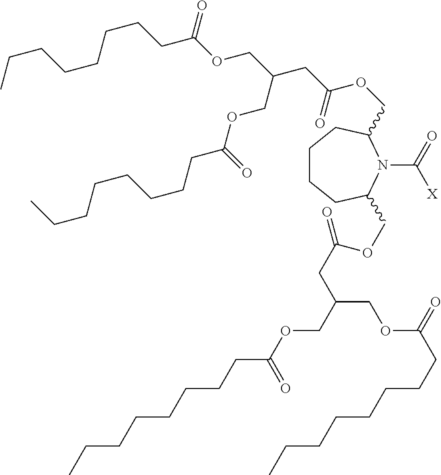

wherein X is as described herein and wavy bonds indicate that any relative or absolute stereo-configuration of the corresponding ring atom can be assumed.

In certain embodiments of formula M2 as described herein, A¹ is (CH₂)₀, A² is (CH₂)₀, A³ is (CH₂)₁, A⁴ is (CH₂)₁, and A⁵ is CH₂—CH═CH—CH₂. For example, in some embodiments, the ionizable cationic lipid has the structure:

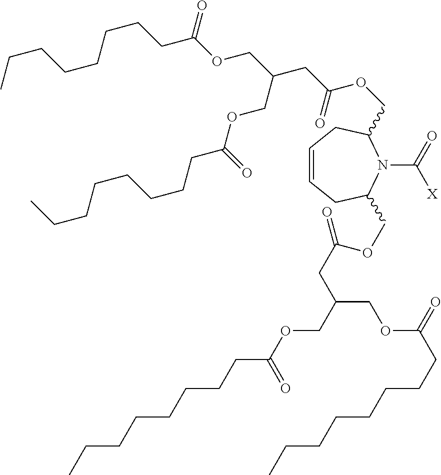

wherein X is as described herein and wavy bonds indicate that any relative or absolute stereo-configuration of the corresponding ring atom can be assumed.

As described above, in some embodiments as described herein, the ionizable cationic lipid has a structure of formula M2, wherein A¹ is (CH₂)₀, A² is (CH₂)₁, A³ is (CH₂)₁, A⁴ is (CH₂)₀, and A⁵ is (CH₂)₁. For example, in some embodiments, the ionizable cationic lipid has the structure:

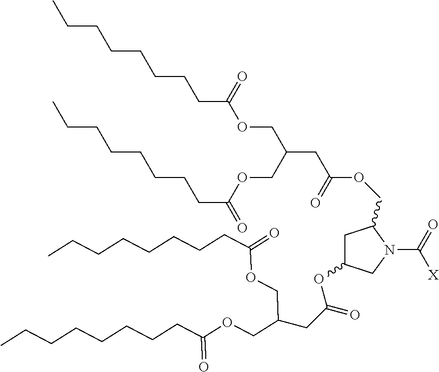

wherein X is as described herein and wavy bonds indicate that any relative or absolute stereo-configuration of the corresponding ring atom can be assumed.

As described above, in some embodiments as described herein, the ionizable cationic lipid has a structure of formula M2, wherein A¹ is (CH₂)₁, A² is (CH₂)₁, A³ is (CH₂)₀, A⁴ is (CH₂)₀, and A⁵ is (CH₂)₀. For example, in some embodiments, the ionizable cationic lipid has the structure:

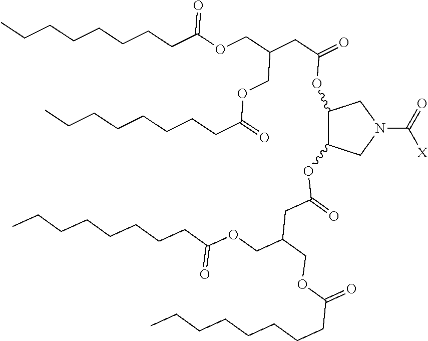

wherein X is as described herein and wavy bonds indicate that any relative or absolute stereo-configuration of the corresponding ring atom can be assumed.

As described above, in some embodiments as described herein, the ionizable cationic lipid has a structure of formula M2, wherein A¹ is (CH₂)₁, A² is (CH₂)₁, A³ is (CH₂)₀, A⁴ is (CH₂)₀, and A⁵ is (CH₂)₁. For example, in some embodiments, the ionizable cationic lipid has the structure:

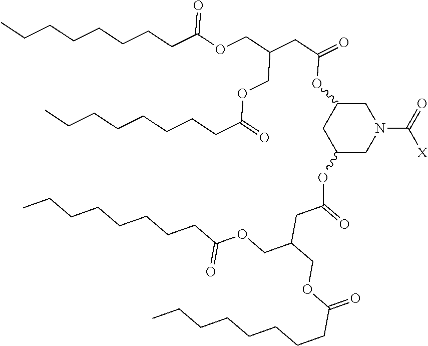

wherein X is as described herein and wavy bonds indicate that any relative or absolute stereo-configuration of the corresponding ring atom can be assumed.

As described above, in some embodiments as described herein, the ionizable cationic lipid has a structure of formula M2 wherein A¹ is (CH₂)₁, A² is (CH₂)₁, A³ is (CH₂)₀, A⁴ is (CH₂)₀, and A⁵ is (CH₂)₂ or CH═CH. For example, in some embodiments as described herein, A¹ is (CH₂)₁, A² is (CH₂)₁, A³ is (CH₂)₀, A⁴ is (CH₂)₀, and A⁵ is (CH₂)₂. In some embodiments as described herein, the ionizable cationic lipid has the structure:

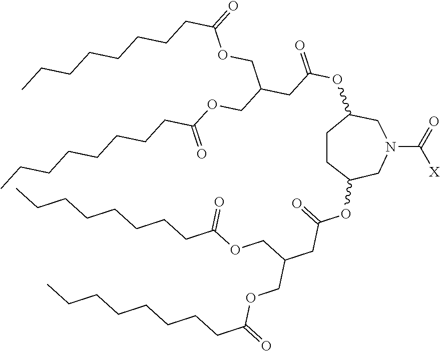

wherein X is as described herein and wavy bonds indicate that any relative or absolute stereo-configuration of the corresponding ring atom can be assumed.

In certain embodiments of formula M2 as described herein, A¹ is (CH₂)₁, A² is (CH₂)₁, A³ is (CH₂)₀, A⁴ is (CH₂)₀, and A⁵ is CH═CH. For example, in some embodiments, the ionizable cationic lipid has the structure:

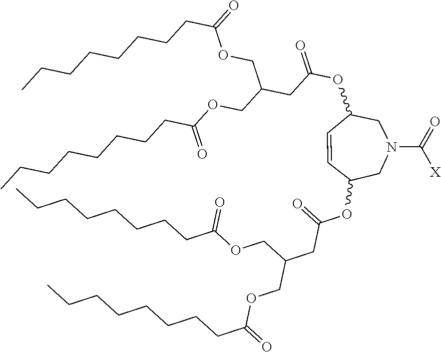

wherein X is as described herein and wavy bonds indicate that any relative or absolute stereo-configuration of the corresponding ring atom can be assumed.

In some embodiments, the ionizable cationic lipid has the structure CICL-207:

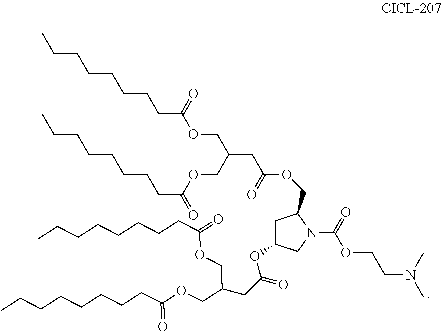 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)C]

CICL-207 is an example of a lipid as disclosed herein in which A¹ and A⁴ are absent, and A², A³, and A⁵ are CH₂, and the tail groups are asymmetrically placed relative to the ring nitrogen in a trans configuration The structure drawn above indicates the absolute stereochemistry for this lipid. Other embodiments include the enantiomer of CICL-207 and mixtures of the enantiomers (e.g., a racemate, or other mixtures with various ratios. Other embodiments include diastereomers of these compounds (e.g., CICL-223 and CICL-224) as well as mixtures including two or more of the various stereoisomers.

In some embodiments, the ionizable cationic lipid has the structure CICL-215:

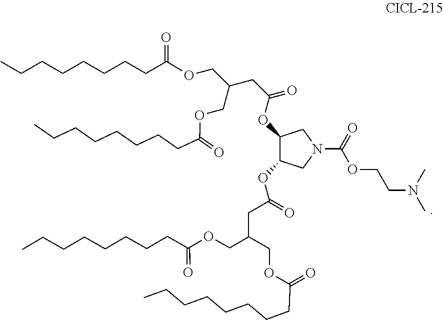 [SMILES: CCCCCCCCC(=O)OCC(COC(=O)CCCCCCCC)CC(=O)O[C@H]1CN(C(=O)OCCN(C)C)C[C@@H]1OC(=O)CC(COC(=O)CCCCCCCC)COC(=O)CCCCCCCC]

CICL-215 is an example of a lipid as disclosed herein in which A³, A⁴, and A⁵ are absent, A¹ and A² are CH₂, and the tail groups are symmetrically placed relative to the ring nitrogen in a trans configuration. The structure drawn above indicates the absolute stereochemistry for this lipid. Other embodiments include its enantiomer and other mixtures thereof. Still other embodiments include diastereomers of these compounds (e.g., CICL-216) as well as mixtures including two or more of the various stereoisomers.

In some embodiments, the ionizable cationic lipid has the structure CICL-216:

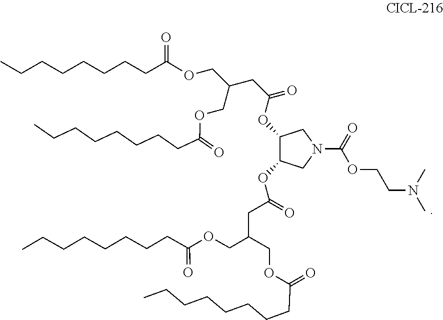 [SMILES: CCCCCCCCC(=O)OCC(COC(=O)CCCCCCCC)CC(=O)O[C@H]1CN(C(=O)OCCN(C)C)C[C@H]1OC(=O)CC(COC(=O)CCCCCCCC)COC(=O)CCCCCCCC]

CICL-216 is an example of a lipid as disclosed herein in which A³, A⁴, and A⁵ are absent, A¹ and A² are CH₂, and the tail groups are symmetrically placed relative to the ring nitrogen in a cis configuration. CICL-216 is symmetric with the two nominal cis configurations being superimposable, due to the tail groups being identical. Other embodiments include diastereomers of this compound (e.g., CICL-215) as well as mixtures including two or more of the various stereoisomers.

In some embodiments, the ionizable cationic lipid has the structure CICL-217:

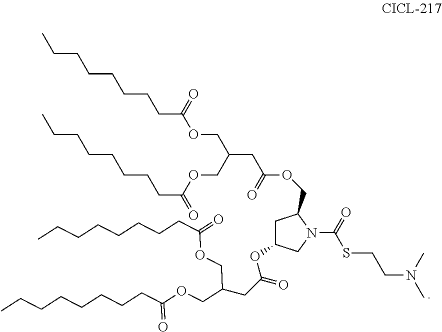 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)SCCN(C)C]

CICL-217 is an example of a lipid as disclosed herein in which A¹ and A⁴ are absent, A², A³, and A⁵ are CH₂, and the tail groups are asymmetrically placed relative to the ring nitrogen in a trans configuration. This lipid differs from CICL-207 in that Y is S instead of O. The structure drawn above indicates the absolute stereochemistry for this lipid. Other embodiments include enantiomer of CICL-217 and diastereomers thereof, and mixtures of stereoisomers (e.g., racemate, or other mixtures of various stereoisomers with various ratios). Other embodiments include diastereomers of these compounds as well as mixtures including two or more of the various stereoisomers.

In some embodiments, the ionizable cationic lipid has the structure CICL-218:

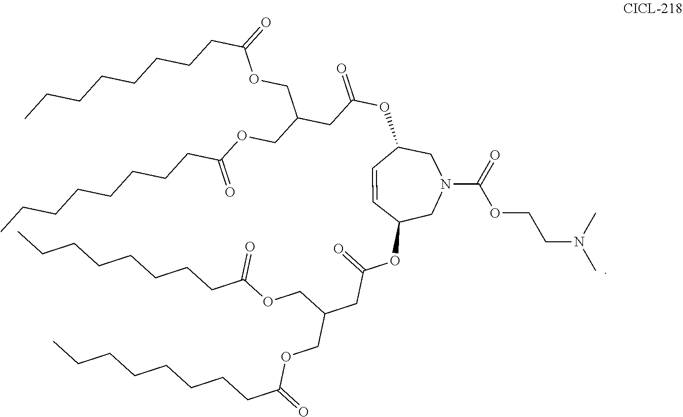

CICL-218 is an example of a lipid as disclosed herein in which A³ and A⁴ are absent, A¹ and A² are CH₂, A⁵ is CH═CH, and the tail groups are symmetrically placed relative to the ring nitrogen in a trans configuration. CICL-218 was synthesized as a racemate so that the structure drawn above indicates the relative stereochemistry for this lipid. Other embodiments include the absolute stereochemistry drawn above, its enantiomer and mixtures thereof. Still other embodiments are diastereomers of these compounds (e.g., CICL-219) as well as mixtures including two or more of the various stereoisomers.

In some embodiments, the ionizable cationic lipid has the structure CICL-219:

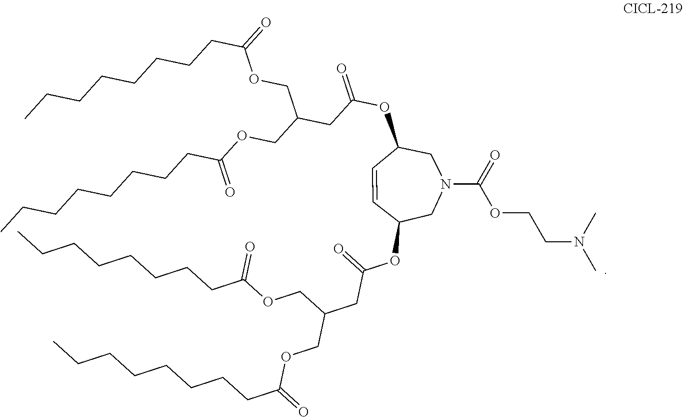

CICL-219 is an example of a lipid as disclosed herein in which A³ and A⁴ are absent, A¹ and A² are CH₂, A⁵ is CH═CH, and the tail groups are symmetrically placed relative to the ring nitrogen in a cis configuration. CICL-219 is symmetric with the two nominal cis configurations being superimposable, due to the tail groups being identical. Other embodiments include diastereomers of this compound (e.g., CICL-218) as well as mixtures including two or more of the various stereoisomers.

In some embodiments, the ionizable cationic lipid has the structure CICL-220:

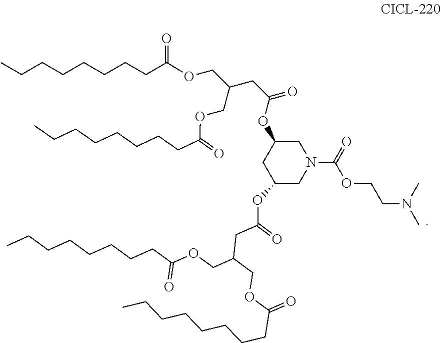 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCCN(C)C)C1]

CICL-220 is an example of a lipid as disclosed herein in which A³ and A⁴ are absent, A¹, A², and A⁵ are CH₂, and the tail groups are symmetrically placed relative to the ring nitrogen in a trans configuration. CICL-220 and the structure drawn above indicates the absolute stereochemistry for this lipid. Other embodiments include its enantiomer and other mixtures thereof. Still other embodiments include diastereomers of these compounds as well as mixtures including two or more of the various stereoisomers.

In some embodiments, the ionizable cationic lipid has the structure CICL-221:

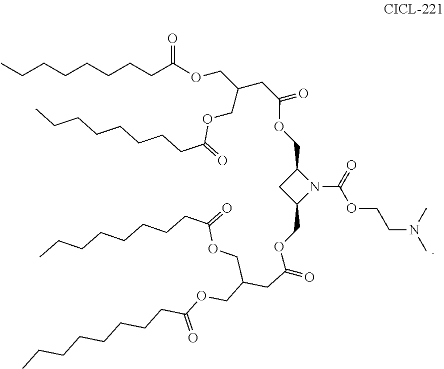 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OCCN(C)C]

CICL-221 is an example of a lipid as disclosed herein in which A¹ and A² are absent, A³, A⁴, and A⁵ are CH₂, and the tail groups are symmetrically placed relative to the ring nitrogen in a cis configuration. CICL-221 is symmetric with the two nominal cis configurations being superimposable, due to the tail groups being identical. Other embodiments include diastereomers of this compound (e.g., CICL-222) as well as mixtures including two or more of the various stereoisomers.

In some embodiments, the ionizable cationic lipid has the structure CICL-222:

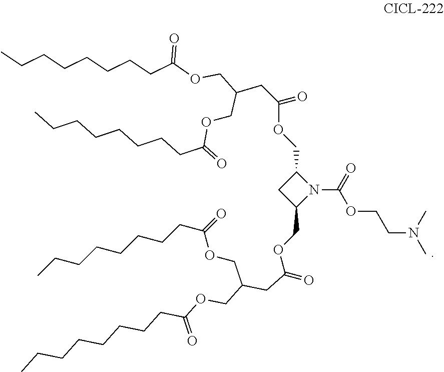 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OCCN(C)C]

CICL-221 is an example of a lipid as disclosed herein in which A¹ and A² are absent, A³, A⁴, and A⁵ are CH₂, and the tail groups are symmetrically placed relative to the ring nitrogen in a trans configuration. CICL-222 was synthesized as a racemate so that the structure drawn above indicates the relative stereochemistry for this lipid. Other embodiments include the absolute stereochemistry drawn above, its enantiomer, and mixtures thereof. Still other embodiments include diastereomers of these compounds (e.g., CICL-221) as well as mixtures including two or more of the various stereoisomers.

In some embodiments, the ionizable cationic lipid has the structure CICL-223:

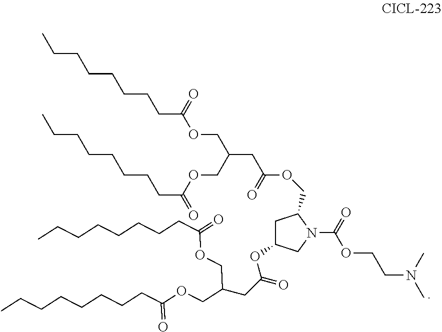 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OCCN(C)C]

CICL-223 is an example of a lipid as disclosed herein in which A¹ and A⁴ are absent, A², A³, and A⁵ are CH₂, and the tail groups are asymmetrically placed relative to the ring nitrogen in a cis configuration. The structure drawn above indicates the absolute stereochemistry for this lipid. Other embodiments include the enantiomer of CICL-223 (CICL-224) and mixtures of the enantiomers (e.g., racemate, or other mixtures with various ratios). Other embodiments include diastereomers of these compounds (e.g., CICL-207 and CICL-225) as well as mixtures including two or more of the various stereoisomers.

In some embodiments, the ionizable cationic lipid has the structure CICL-224:

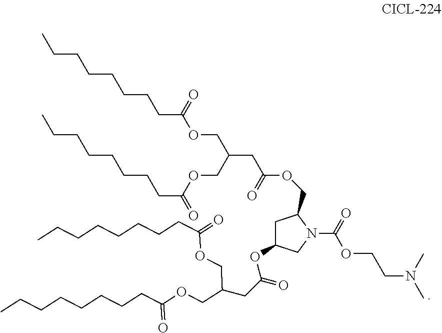 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OCCN(C)C]

CICL-224 is an example of a lipid as disclosed herein in which A¹ and A⁴ are absent, A², A³, and A⁵ are CH₂, and the tail groups are asymmetrically placed relative to the ring nitrogen in a cis configuration. The structure drawn above indicates the absolute stereochemistry for this lipid. Other embodiments include the enantiomer of CICL-224 (CICL-223) and mixtures of the enantiomers (e.g., racemate, or other mixtures with various ratios) Other embodiments include diastereomers of these compounds (e.g., CICL-207 and CICL-225) as well as mixtures including two or more of the various stereoisomers.

In some embodiments, the ionizable cationic lipid has the structure CICL-225:

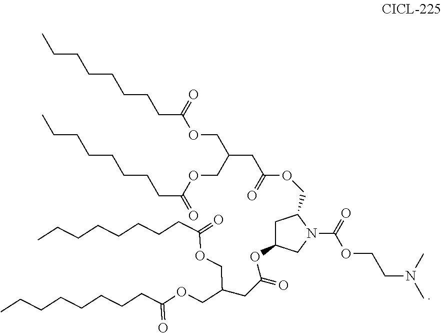 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCN(C)C]

CICL-225 is an example of a lipid as disclosed herein in which A¹ and A⁴ are absent, A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-225 (CICL-207) and mixtures of the enantiomers (e.g., racemate, or other mixtures with various ratios). Other embodiments include diastereomers of these compounds (e.g., CICL-223 and CICL-224) as well as mixtures including two or more of the various stereoisomers.

In some embodiments, the ionizable cationic lipid has the structure CICL-238:

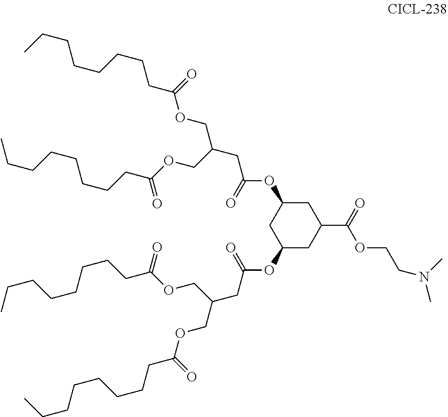 [SMILES: CCCCCCCCC(=O)OCC(COC(=O)CCCCCCCC)CC(=O)O[C@@H]1CC(C(=O)OCCN(C)C)C[C@H](OC(=O)CC(COC(=O)CCCCCCCC)COC(=O)CCCCCCCC)C1]

CICL-238 is an example of a lipid as disclosed herein in which A¹, A², and A⁵ are CH₂, A³ and A⁴ are absent, and the tail groups are symmetrically placed relative to the ring nitrogen in a cis configuration. CICL-238 is symmetric with the two nominal cis configurations being superimposable, due to the tail groups being identical. Other embodiments include diastereomers of this compound (e.g., CICL-220) as well as mixtures including two or more of the various stereoisomers.

In some embodiments, the ionizable cationic lipid has the structure CICL-239:

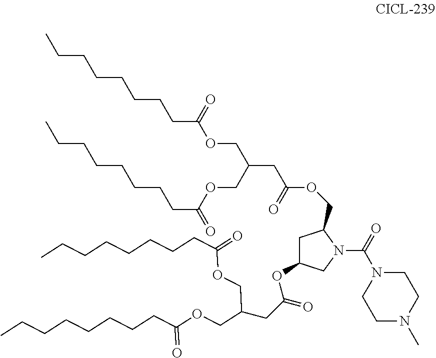 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)N1CCN(C)CC1]

CICL-239 is an example of a lipid as disclosed herein in which A¹ and A⁴ are absent, A², A³, and A⁵ are CH₂, and the tail groups are asymmetrically placed relative to the ring nitrogen in a cis configuration. Differing from many of the exemplified lipids the group Y of CICL-239 is N-methylpiperazine. The structure drawn above indicates the absolute stereochemistry for this lipid. Other embodiments include the enantiomer of CICL-239 and mixtures of the enantiomers (e.g., racemate, or other mixtures with various ratios). Other embodiments include diastereomers of these compounds as well as mixtures including two or more of the various stereoisomers.

In some embodiments, the ionizable cationic lipid has the structure CICL-242:

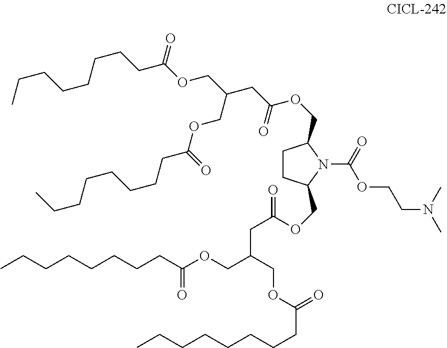 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCN(C)C]

CICL-242 is an example of a lipid as disclosed herein in which A¹ and A² are absent, A³ and A⁴ are CH₂, and A⁵ is CH₂CH₂, and the tail groups are symmetrically placed relative to the ring nitrogen in a cis configuration. CICL-242 is symmetric with the two nominal cis configurations being superimposable, due to the tail groups being identical. Other embodiments include diastereomers of this compound (e.g., CICL-243) as well as mixtures including two or more of the various stereoisomers.

In some embodiments, the ionizable cationic lipid has the structure CICL-243:

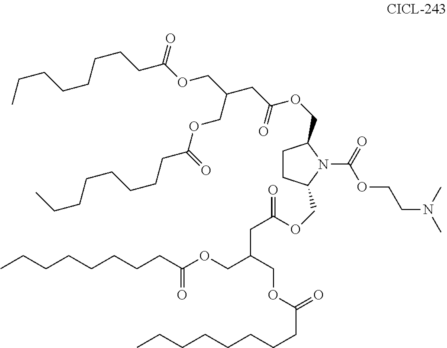 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCN(C)C]

CICL-243 is an example of a lipid as disclosed herein in which A¹ and A² are absent, A³ and A⁴ are CH₂, and A⁵ is CH₂CH₂, and the tail groups are symmetrically placed relative to the ring nitrogen in a trans configuration. CICL-243 is an optical antipode, so that the structure drawn above indicates the absolute stereochemistry for this lipid. Other embodiments include: its enantiomer, and mixtures of the enantiomers (e.g., racemate, or other mixtures with various ratios). Still other embodiments include diastereomers of these compounds (e.g., CICL-242) as well as mixtures including two or more of the various stereoisomers.

In some embodiments, the ionizable cationic lipid has the structure CICL-244:

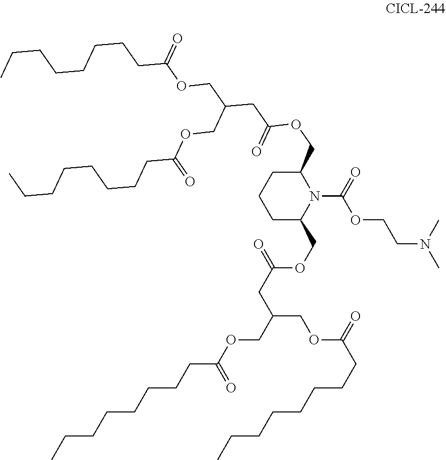 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCN(C)C]

CICL-244 is an example of a lipid as disclosed herein in which A¹ and A² are absent, A³ and A⁴ are CH₂, and A⁵ is CH₂CH₂CH₂, and the tail groups are symmetrically placed relative to the ring nitrogen in a cis configuration. CICL-244 is symmetric with the two nominal cis configurations being superimposable, due to the tail groups being identical. Other embodiments include diastereomers of this compound (e.g., CICL-245) as well as mixtures including two or more of the various stereoisomers.

In some embodiments, the ionizable cationic lipid has the structure CICL-245:

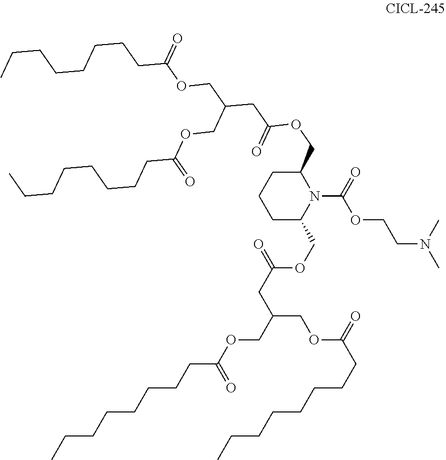 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCN(C)C]

CICL-245 is an example of a lipid as disclosed herein in which A¹ and A² are absent, A³ and A⁴ are CH₂, and A⁵ is CH₂CH₂CH₂, and the tail groups are symmetrically placed relative to the ring nitrogen in a trans configuration. CICL-245 is a racemate, so that the structure drawn above indicates the relative stereochemistry for this lipid. Other embodiments include: its enantiomers, and mixtures thereof. Still other embodiments include diastereomers of these compounds (e.g., CICL-244) as well as mixtures including two or more of the various stereoisomers.

In some embodiments, the ionizable cationic lipid has the structure CICL-246:

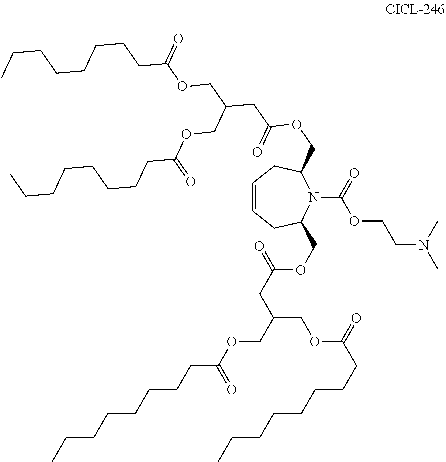 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCCN(C)C]

CICL-246 is an example of a lipid as disclosed herein in which A¹ and A² are absent, A³ and A⁴ are CH₂, and A⁵ is CH₂CH═CHCH₂, and the tail groups are symmetrically placed relative to the ring nitrogen in a cis configuration. CICL-246 is symmetric with the two nominal cis configurations being superimposable, due to the tail groups being identical. Other embodiments include diastereomers of this compound (e.g., CICL-247) as well as mixtures including two or more of the various stereoisomers.

In some embodiments, the ionizable cationic lipid has the structure CICL-247:

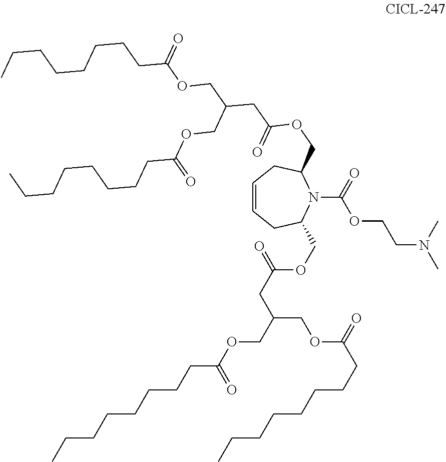 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCCN(C)C]

CICL-247 is an example of a lipid as disclosed herein in which A¹ and A² are absent, A³ and A⁴ are CH₂, and A⁵ is CH₂CH═CHCH₂, and the tail groups are symmetrically placed relative to the ring nitrogen in a trans configuration. CICL-247 is a racemate, so that the structure drawn above indicates the relative stereochemistry for this lipid. Other embodiments include: its enantiomers, and mixtures thereof. Still other embodiments include diastereomers of these compounds (e.g., CICL-246) as well as mixtures including two or more of the various stereoisomers.

In some embodiments, the ionizable cationic lipid has the structure CICL-248:

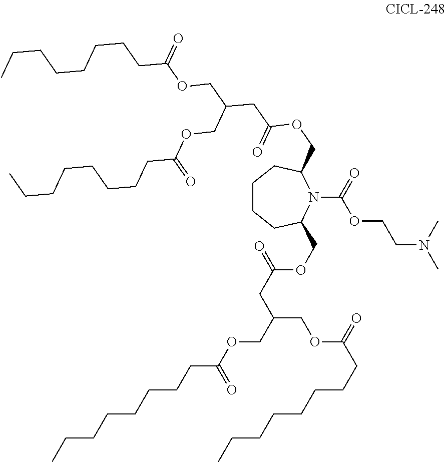 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCCN(C)C]

CICL-248 is an example of a lipid as disclosed herein in which A¹ and A² are absent, A³ and A⁴ are CH₂, and A⁵ is CH₂CH₂CH₂CH₂, and the tail groups are symmetrically placed relative to the ring nitrogen in a cis configuration. CICL-248 is symmetric with the two nominal cis configurations being superimposable, due to the tail groups being identical. Other embodiments include diastereomers of this compound (e.g., CICL-249) as well as mixtures including two or more of the various stereoisomers.

In some embodiments, the ionizable cationic lipid has the structure CICL-249:

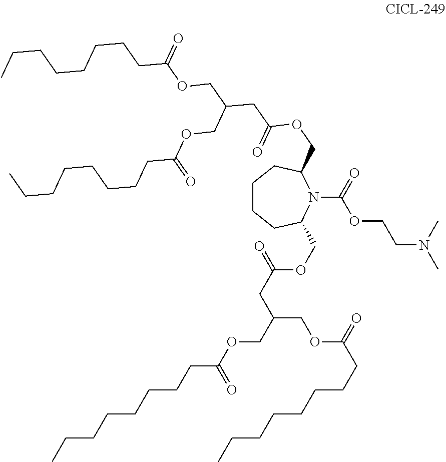 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCN(C)C]

CICL-249 is an example of a lipid as disclosed herein in which A¹ and A² are absent, A³ and A⁴ are CH₂, and A⁵ is CH₂CH₂CH₂CH₂, and the tail groups are symmetrically placed relative to the ring nitrogen in a trans configuration. CICL-249 is a racemate, so that the structure drawn above indicates the relative stereochemistry for this lipid. Other embodiments include: its enantiomers, and mixtures thereof. Still other embodiments include diastereomers of these compounds (e.g., CICL-248) as well as mixtures including two or more of the various stereoisomers.

In some embodiments, the ionizable cationic lipid has the structure CICL-207-91:

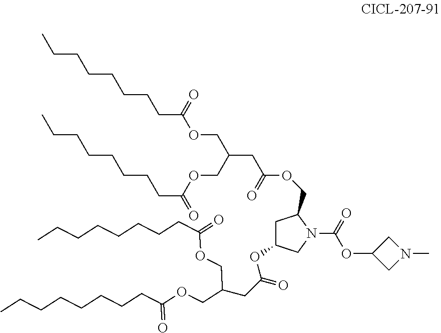 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CN(C)C1]

CICL-207-91 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-91 and mixtures of the enantiomers (e.g., a racemate, or other mixtures with various ratios). Further embodiments include or are diastereomers of CICL-207-91, and mixtures thereof.

In some embodiments, the ionizable cationic lipid has the structure CICL-207-92:

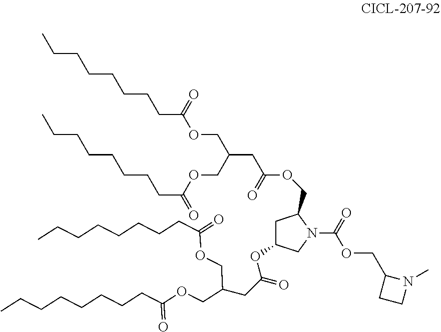 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCN1C]

CICL-207-92 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-92 and mixtures of the enantiomers (e.g., a racemate, or other mixtures with various ratios). Further embodiments include or are diastereomers of CICL-207-92, and mixtures thereof.

In some embodiments, the ionizable cationic lipid has the structure CICL-207-93:

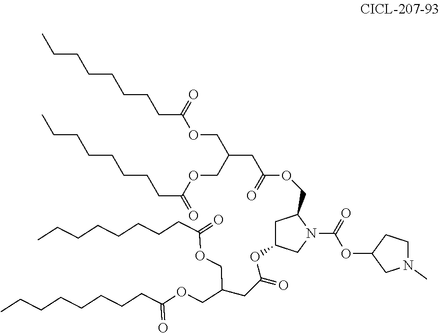 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCN(C)C1]

CICL-207-93 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-93 and mixtures of the enantiomers (e.g., a racemate, or other mixtures with various ratios). Further embodiments include or are diastereomers of CICL-207-93, and mixtures thereof.

In some embodiments, the ionizable cationic lipid has the structure CICL-207-94:

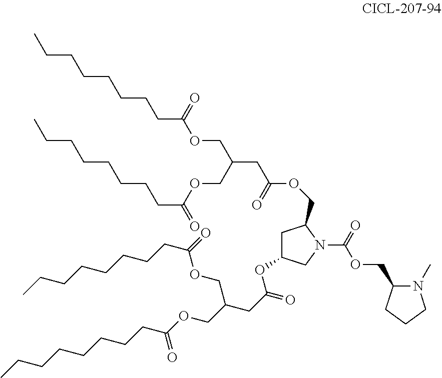 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC[C@@H]1CCCN1C]

CICL-207-94 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-94 and mixtures of the enantiomers (e.g., a racemate, or other mixtures with various ratios). Further embodiments include or are diastereomers of CICL-207-94, and mixtures thereof.

In some embodiments, the ionizable cationic lipid has the structure CICL-207-95:

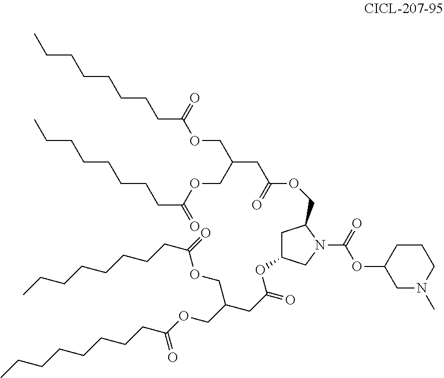 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCCN(C)C1]

CICL-207-95 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-95 and mixtures of the enantiomers (e.g., a racemate, or other mixtures with various ratios). Further embodiments include or are diastereomers of CICL-207-95, and mixtures thereof.

In some embodiments, the ionizable cationic lipid has the structure CICL-207-96:

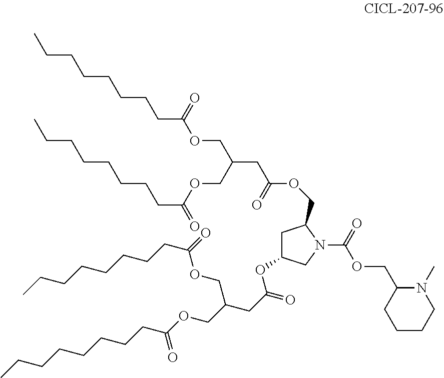 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCCN1C]

CICL-207-96 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-96 and mixtures of the enantiomers (e.g., a racemate, or other mixtures with various ratios). Further embodiments include or are diastereomers of CICL-207-96, and mixtures thereof.

In some embodiments, the ionizable cationic lipid has the structure CICL-207-97:

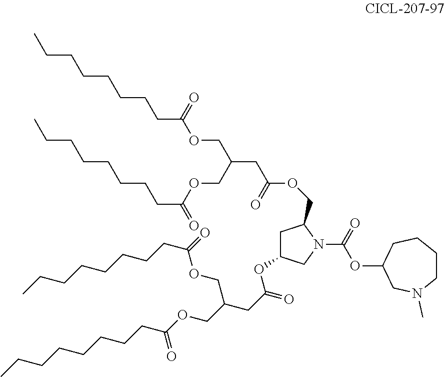 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCCCN(C)C1]

CICL-207-97 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-97 and mixtures of the enantiomers (e.g., a racemate, or other mixtures with various ratios). Further embodiments include or are diastereomers of CICL-207-97, and mixtures thereof.

In some embodiments, the ionizable cationic lipid has the structure CICL-207-98:

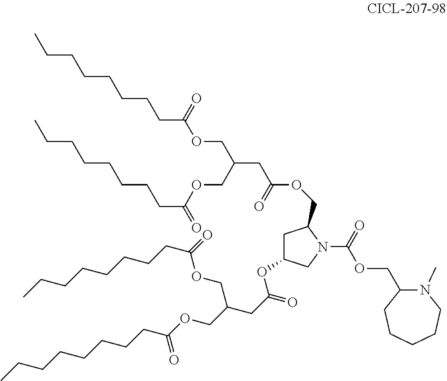 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCCCN1C]

CICL-207-98 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-98 and mixtures of the enantiomers (e.g., a racemate, or other mixtures with various ratios). Further embodiments include or are diastereomers of CICL-207-98, and mixtures thereof.

In some embodiments, the ionizable cationic lipid has the structure CICL-207-99:

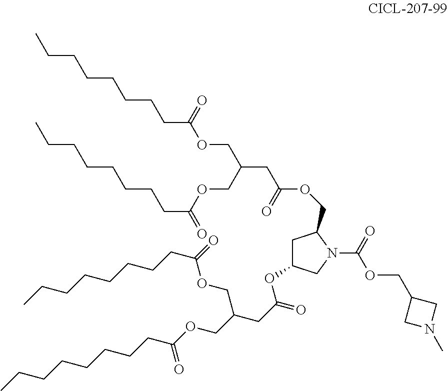 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CN(C)C1]

CICL-207-99 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-99 and mixtures of the enantiomers (e.g., a racemate, or other mixtures with various ratios). Further embodiments include or are diastereomers of CICL-207-99, and mixtures thereof.

In some embodiments, the ionizable cationic lipid has the structure CICL-207-100:

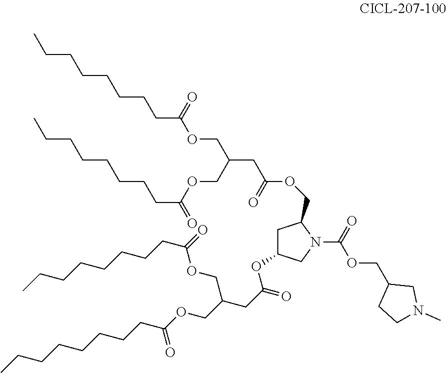 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCN(C)C1]

CICL-207-100 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-100 and mixtures of the enantiomers (e.g., a racemate, or other mixtures with various ratios). Further embodiments include or are diastereomers of CICL-207-100, and mixtures thereof.

In some embodiments, the ionizable cationic lipid has the structure CICL-207-101:

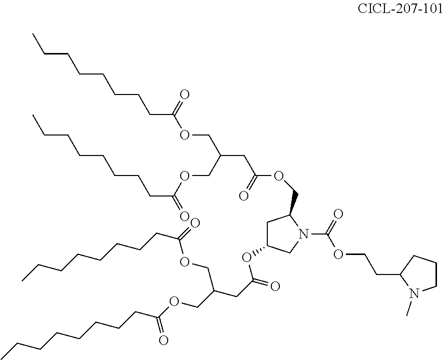 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCC1CCCN1C]

CICL-207-101 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-101 and mixtures of the enantiomers (e.g., a racemate, or other mixtures with various ratios). Further embodiments include or are diastereomers of CICL-207-101, and mixtures thereof.

In some embodiments, the ionizable cationic lipid has the structure CICL-207-102:

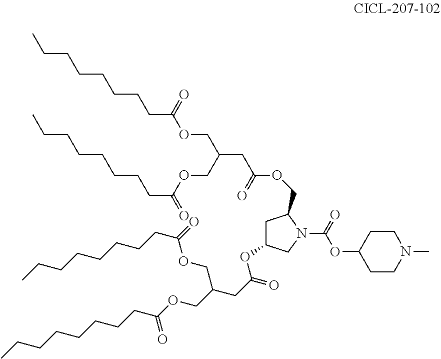 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCN(C)CC1]

CICL-207-102 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-102 and mixtures of the enantiomers (e.g., a racemate, or other mixtures with various ratios). Further embodiments include or are diastereomers of CICL-207-102, and mixtures thereof.

In some embodiments, the ionizable cationic lipid has the structure CICL-207-103:

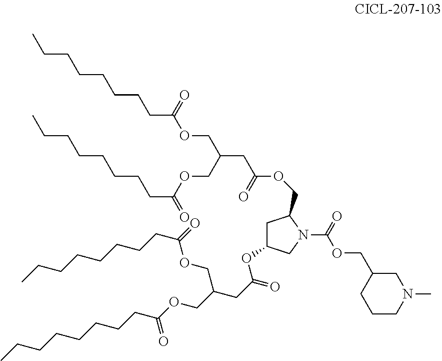 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCN(C)C1]

CICL-207-103 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-103 and mixtures of the enantiomers (e.g., a racemate, or other mixtures with various ratios). Further embodiments include or are diastereomers of CICL-207-103, and mixtures thereof.

In some embodiments, the ionizable cationic lipid has the structure CICL-207-104:

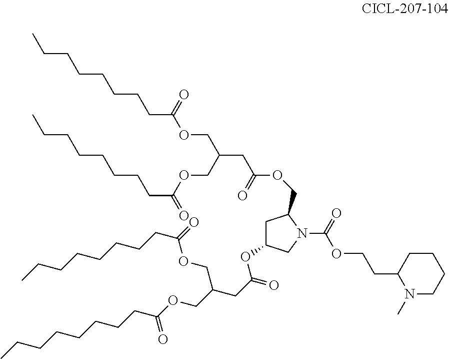 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCC1CCCCN1C]

CICL-207-104 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-104 and mixtures of the enantiomers (e.g., a racemate, or other mixtures with various ratios). Further embodiments include or are diastereomers of CICL-207-104, and mixtures thereof.

In some embodiments, the ionizable cationic lipid has the structure CICL-207-105:

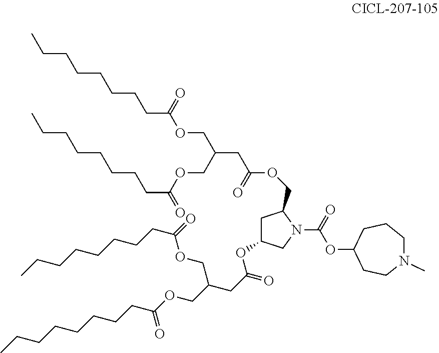 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCCN(C)CC1]

CICL-207-105 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-105 and mixtures of the enantiomers (e.g., a racemate, or other mixtures with various ratios). Further embodiments include or are diastereomers of CICL-207-105, and mixtures thereof.

In some embodiments, the ionizable cationic lipid has the structure CICL-207-106:

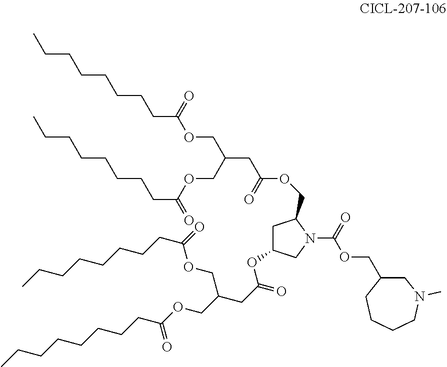 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCCN(C)C1]

CICL-207-106 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-106 and mixtures of the enantiomers (e.g., a racemate, or other mixtures with various ratios). Further embodiments include or are diastereomers of CICL-207-106, and mixtures thereof.

In some embodiments, the ionizable cationic lipid has the structure CICL-207-107:

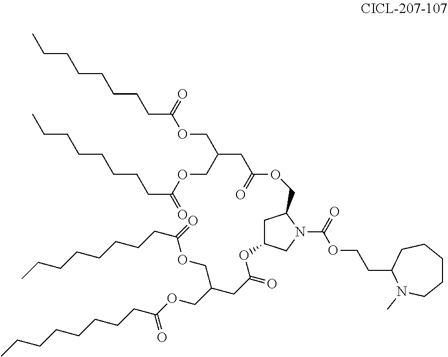 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCC1CCCCCN1C]

CICL-207-107 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-107 and mixtures of the enantiomers (e.g., a racemate, or other mixtures with various ratios). Further embodiments include or are diastereomers of CICL-207-107, and mixtures thereof.

In some embodiments, the ionizable cationic lipid has a structure selected from the following:

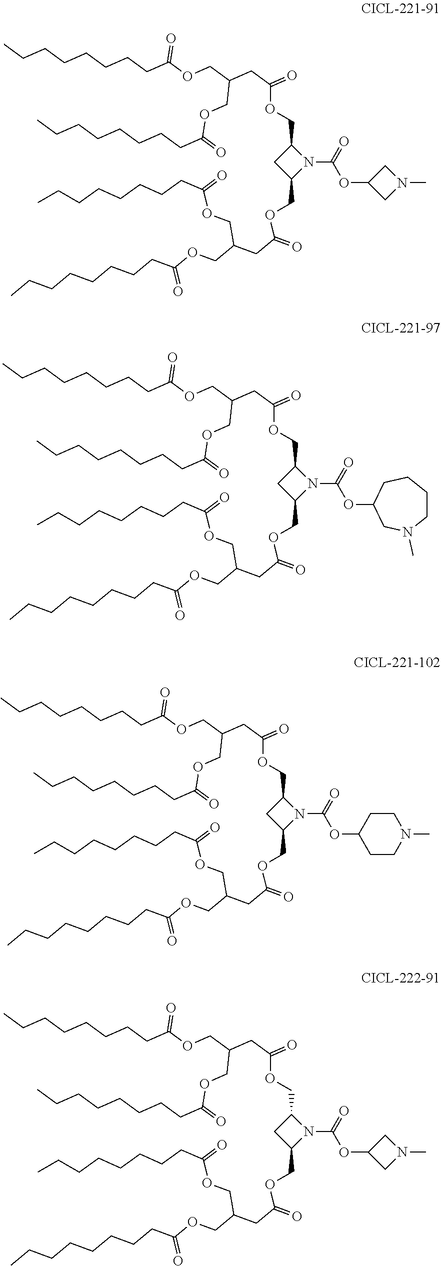

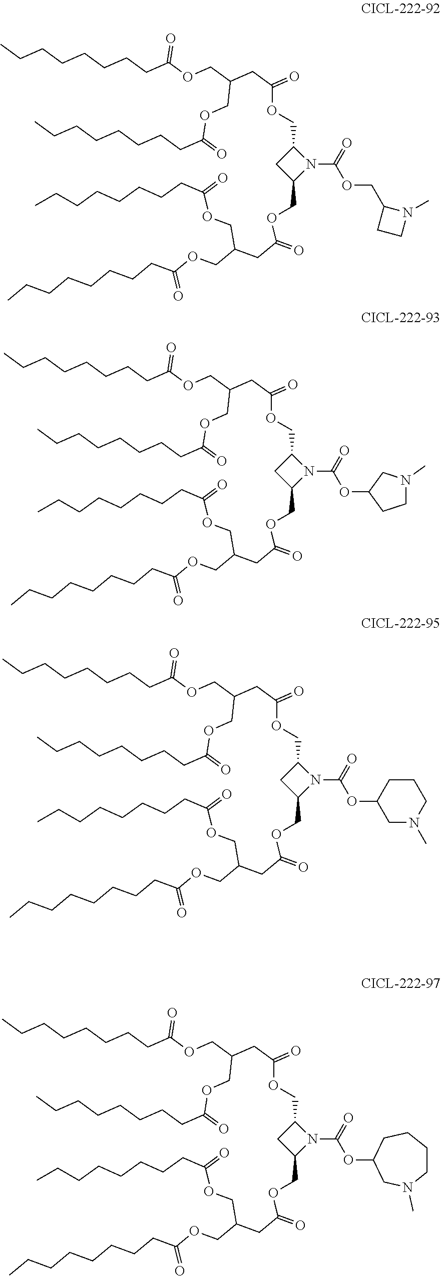

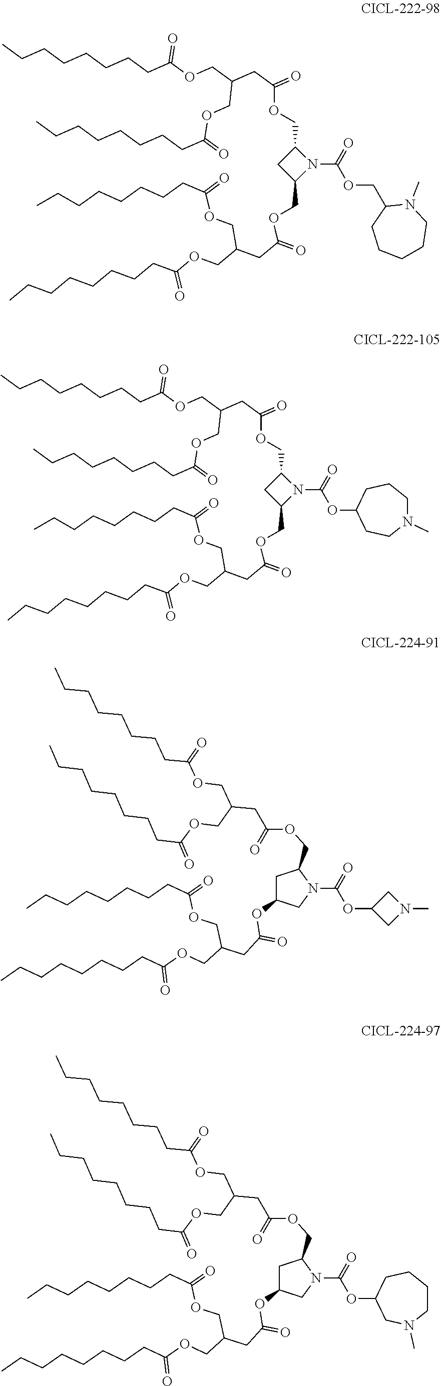

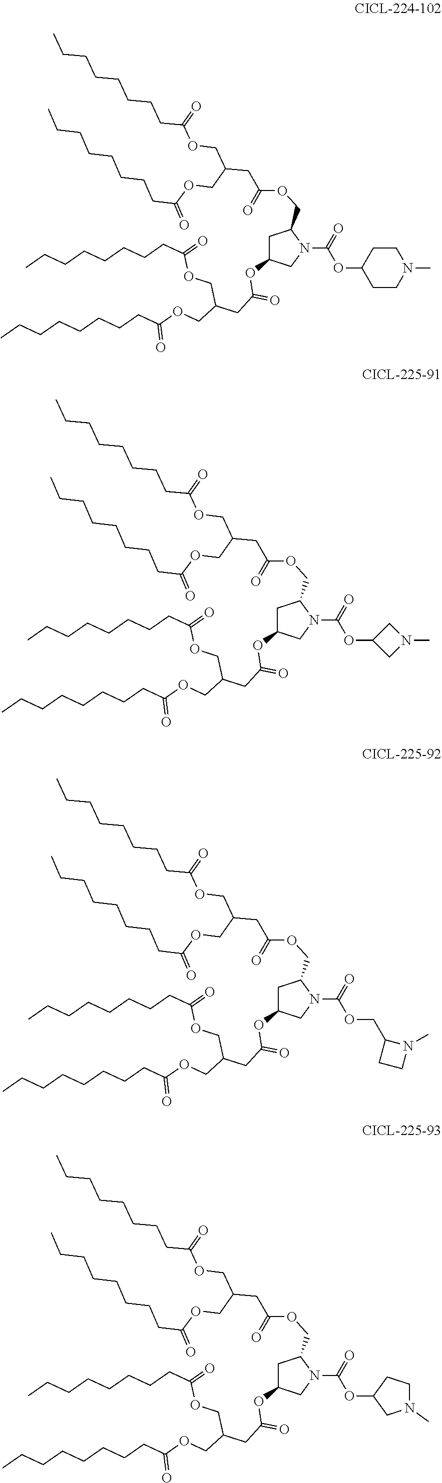

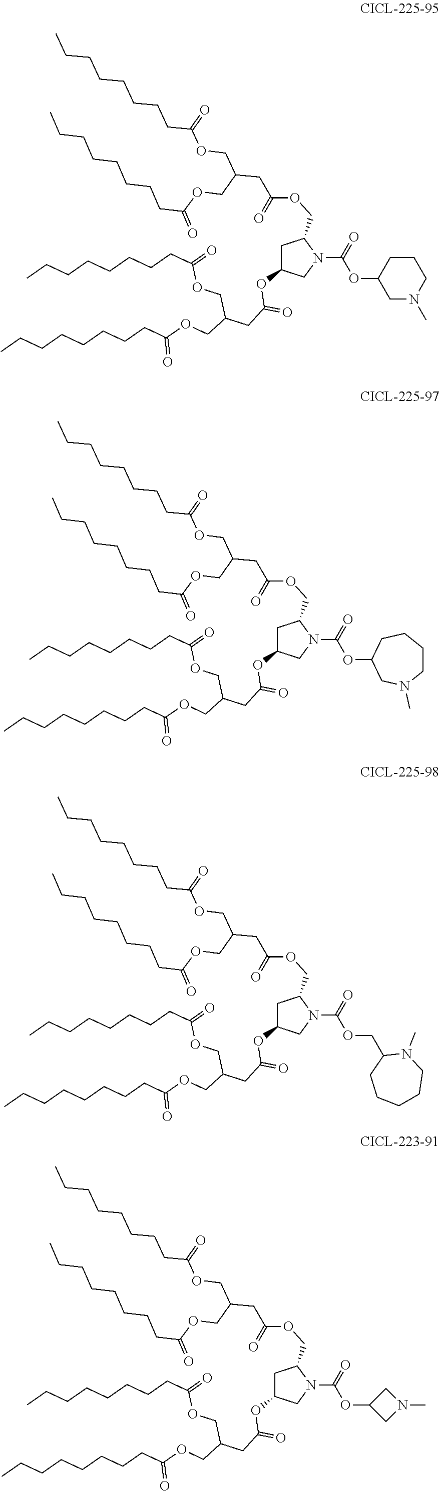

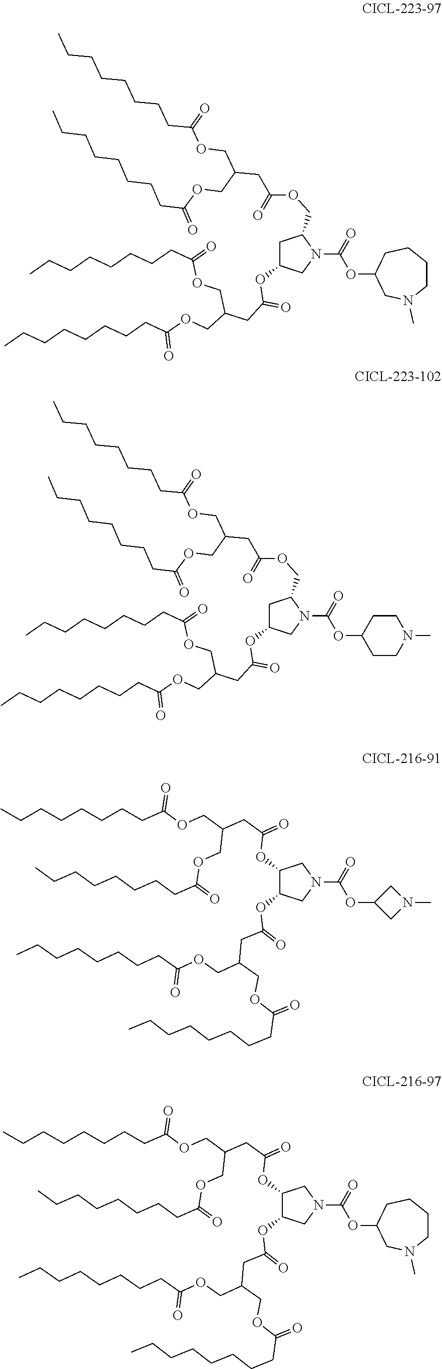

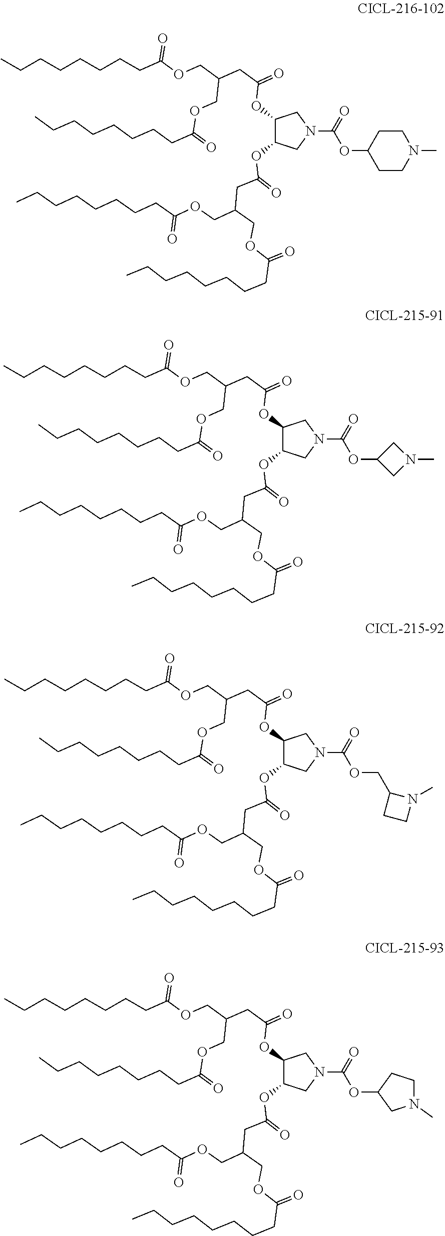

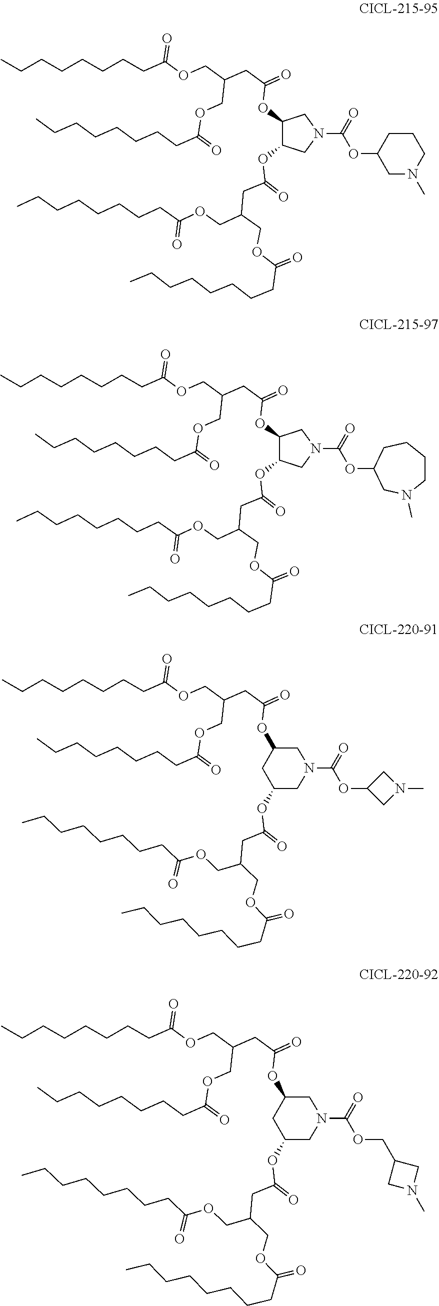

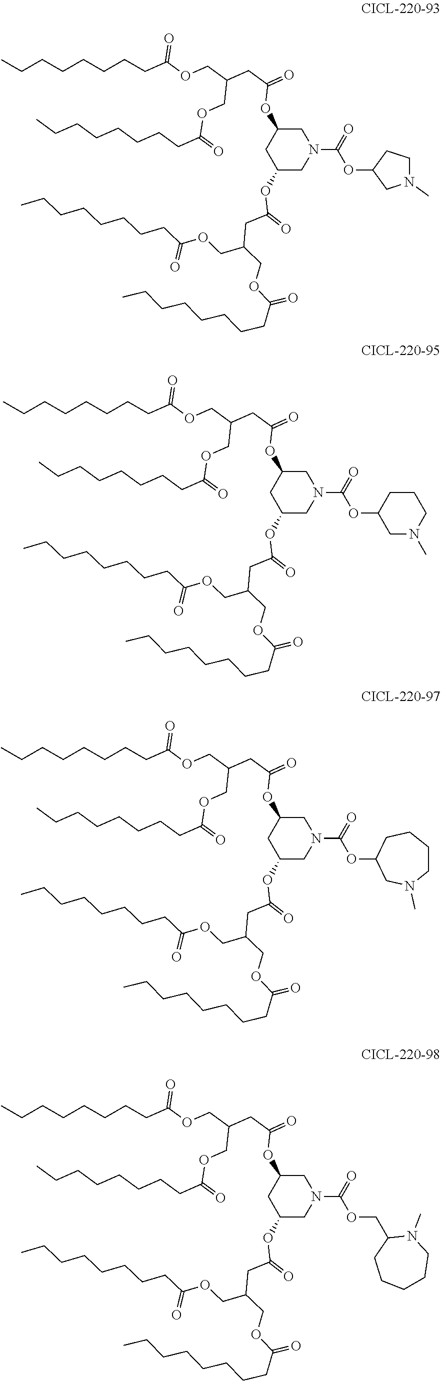

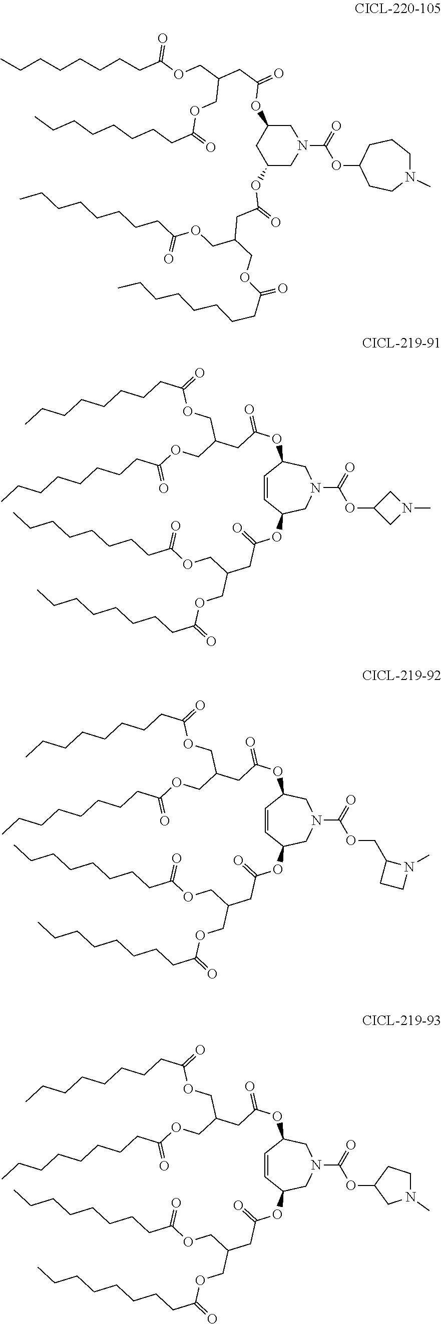

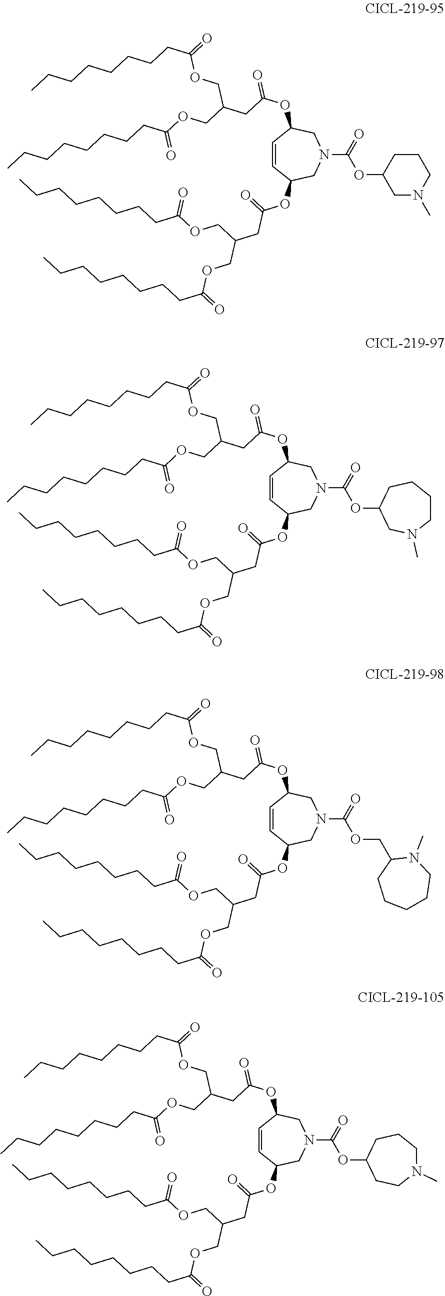

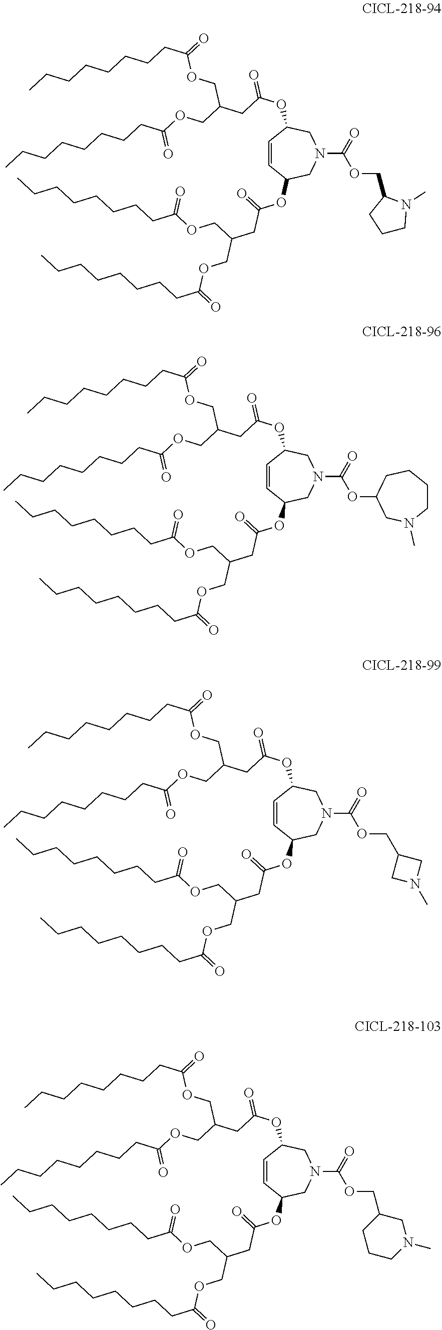

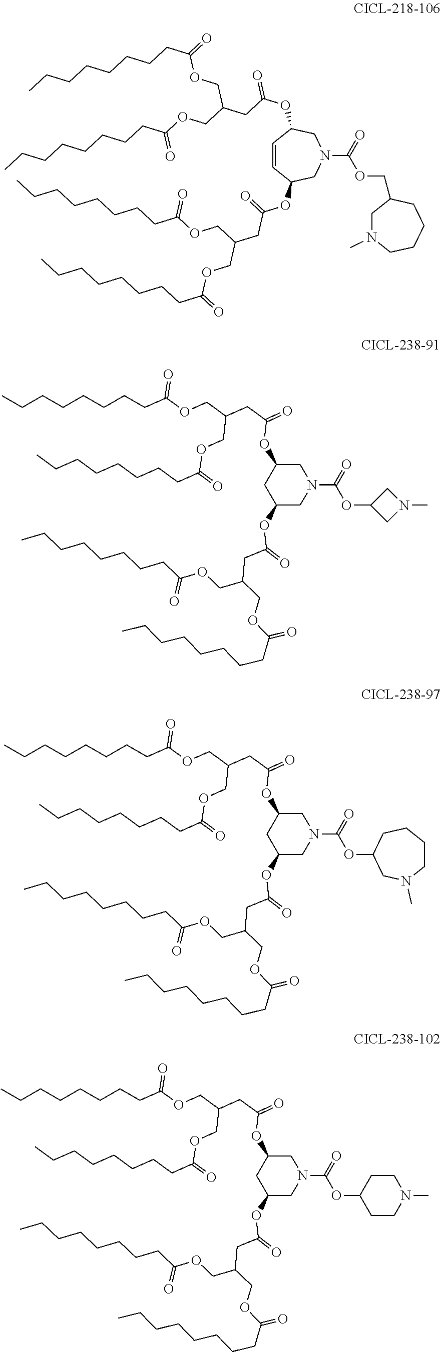

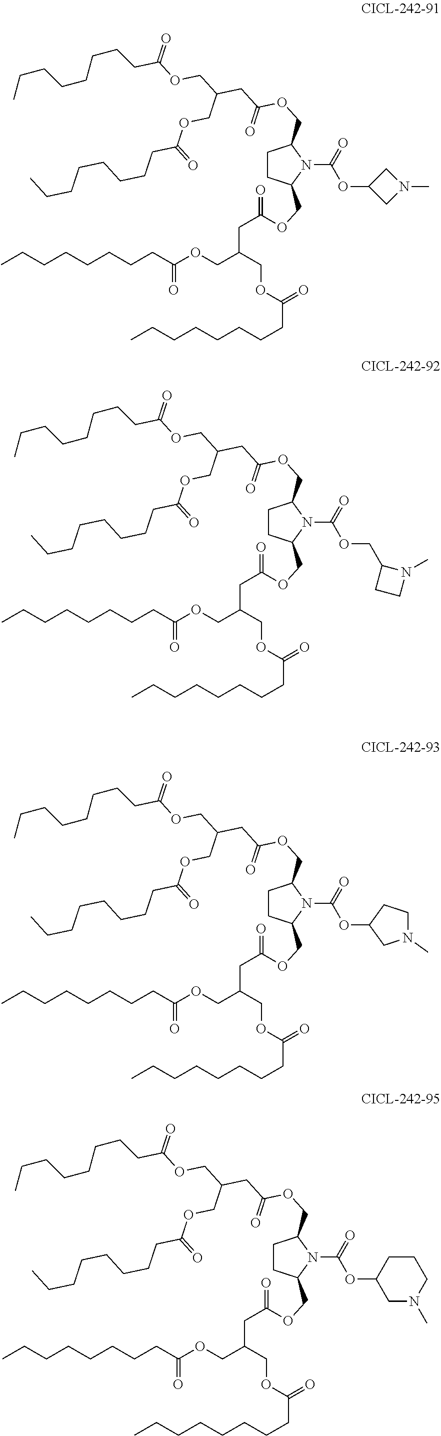

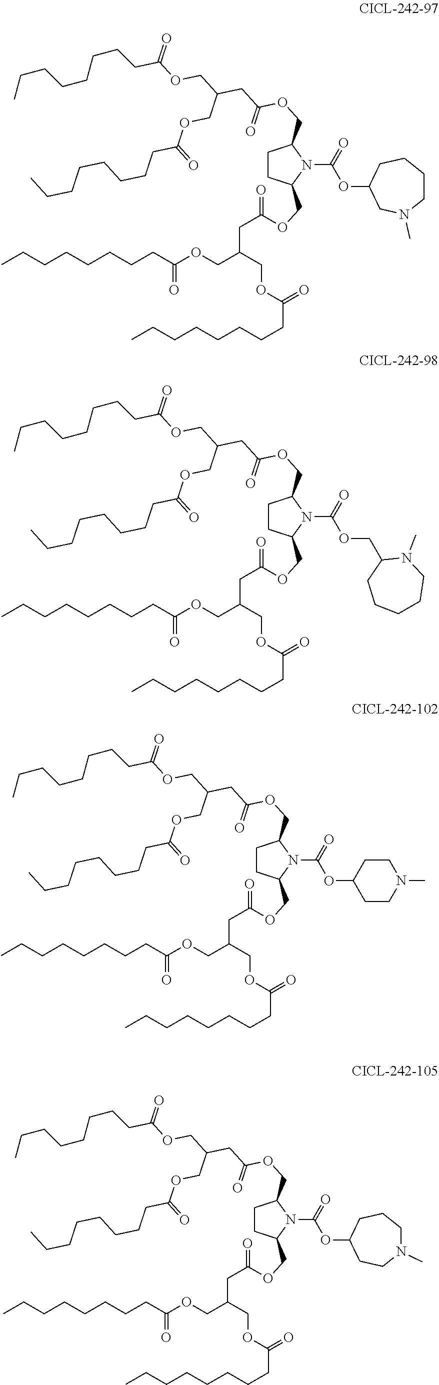

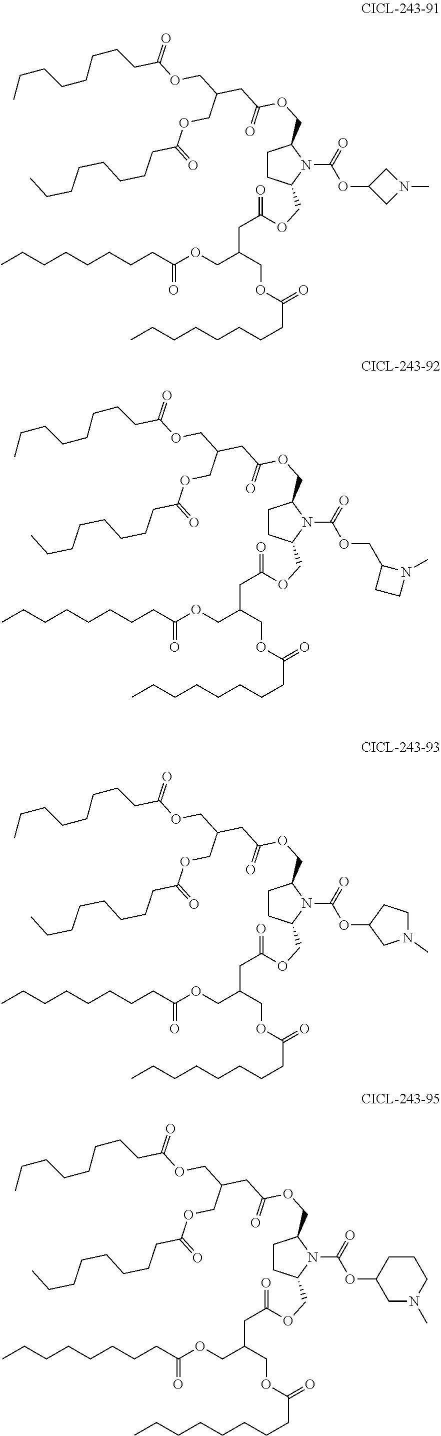

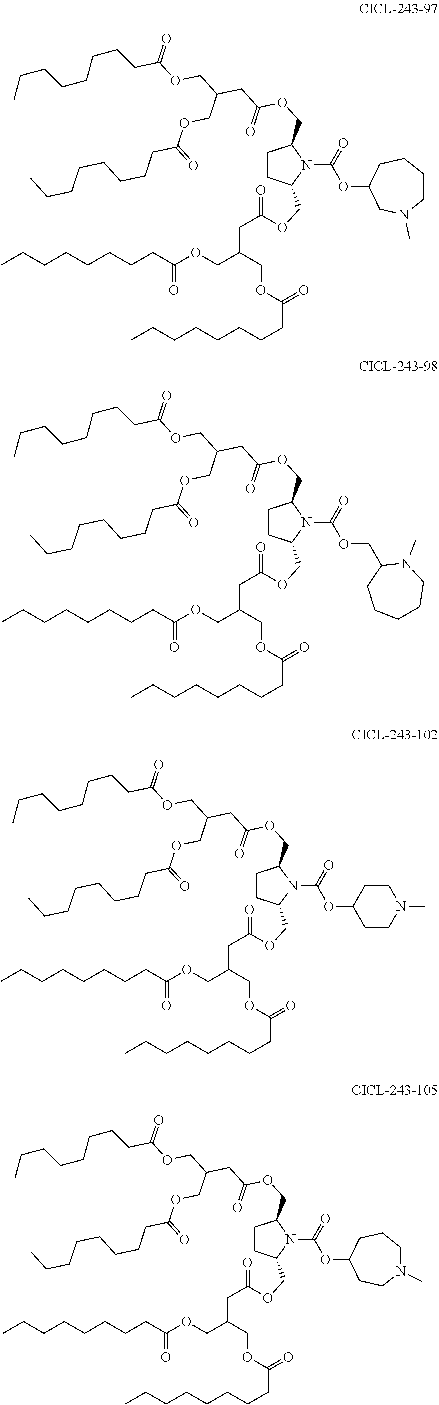

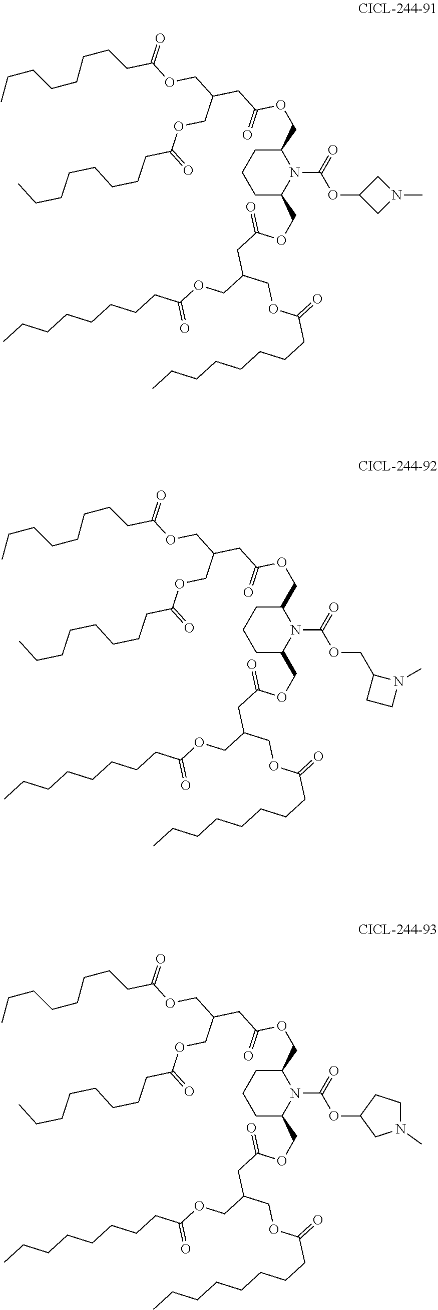

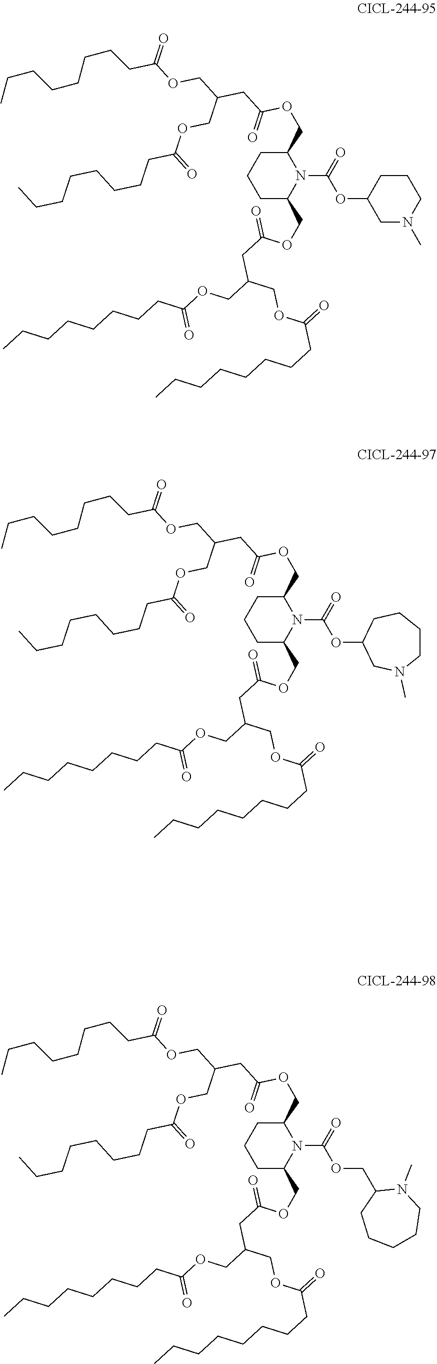

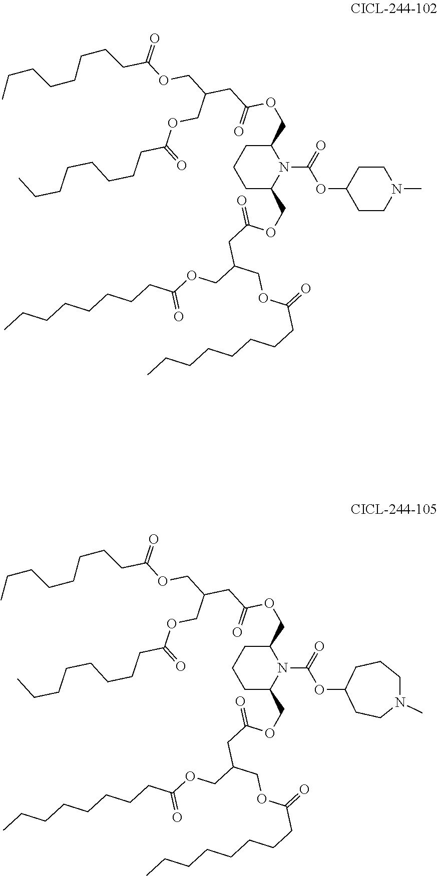

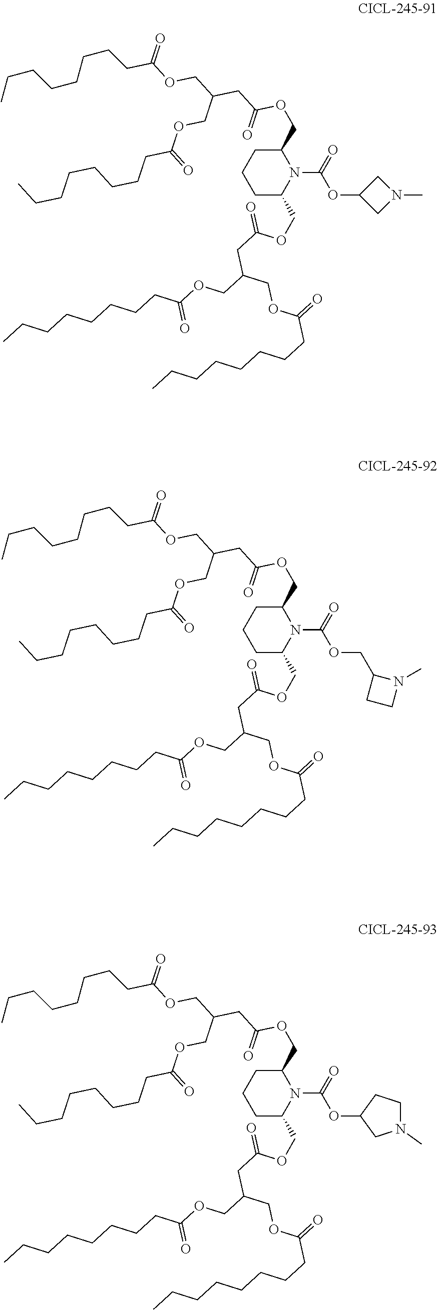

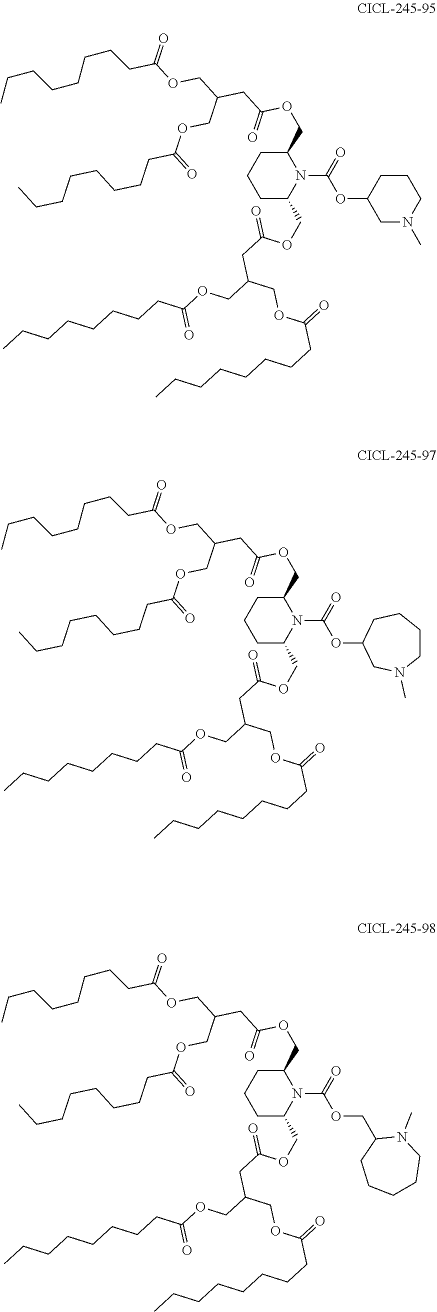

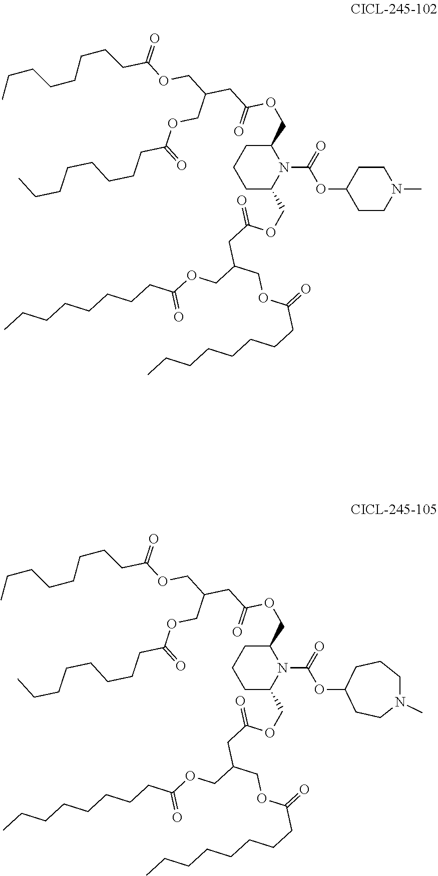

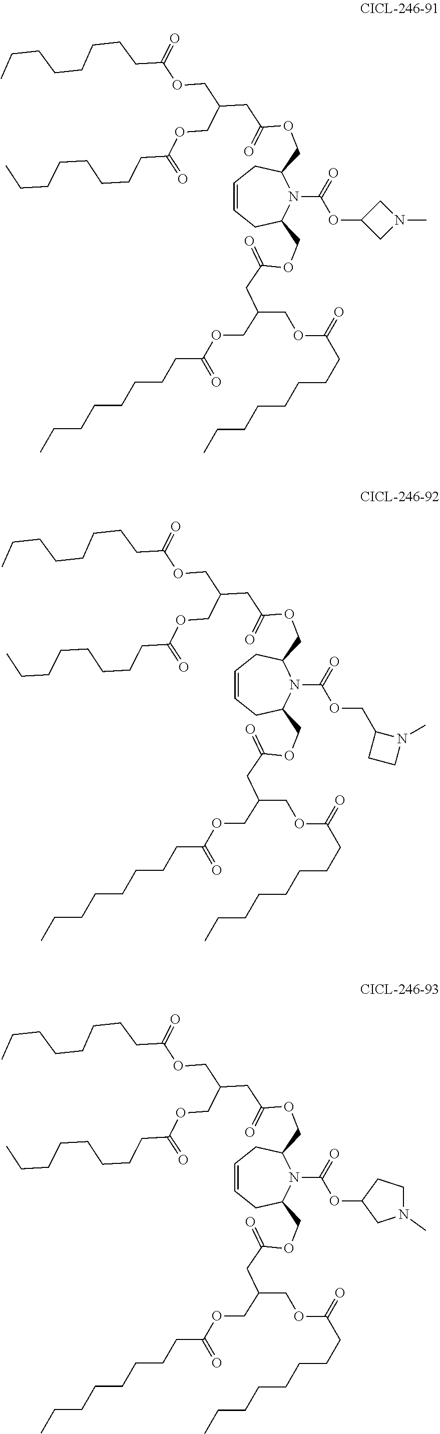

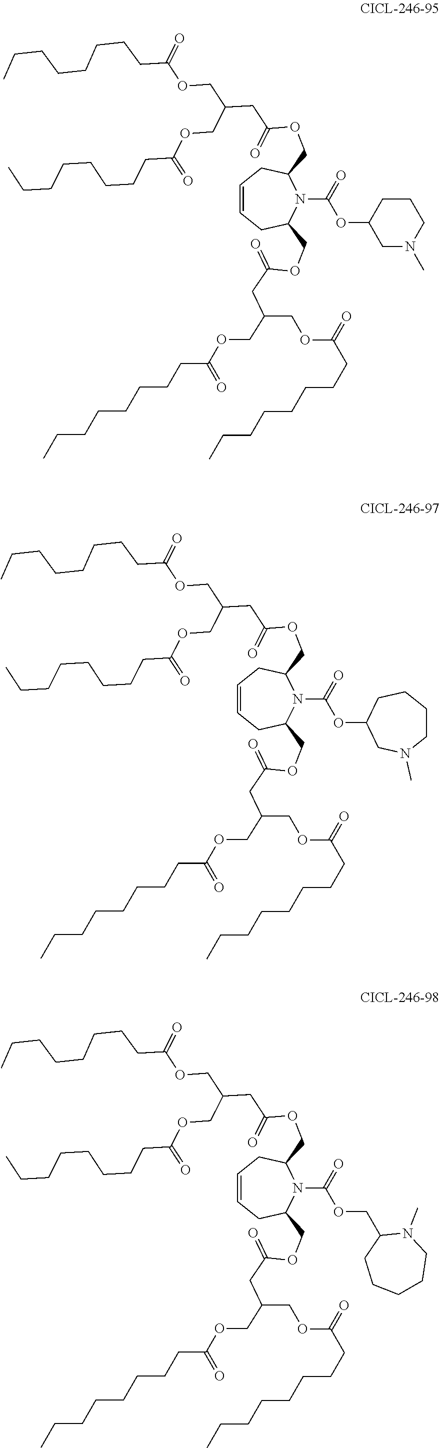

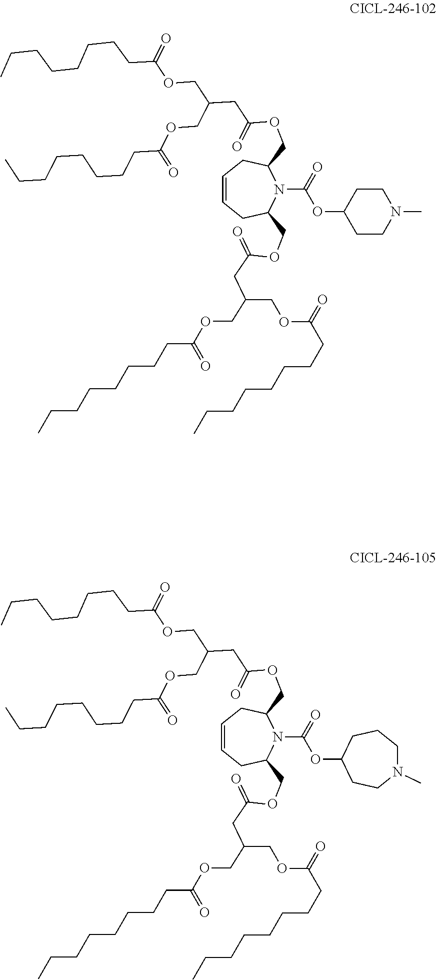

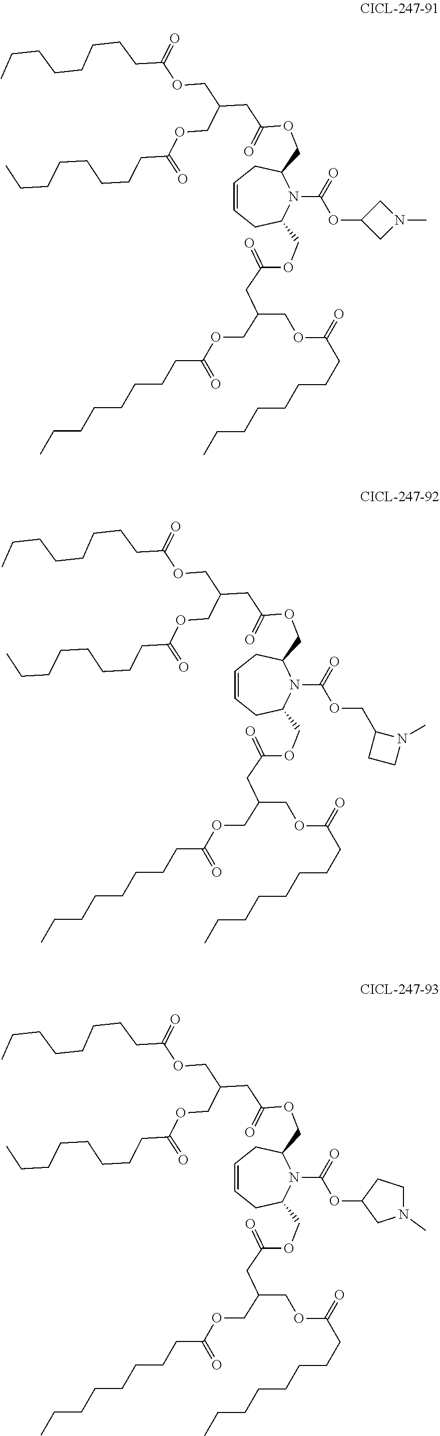

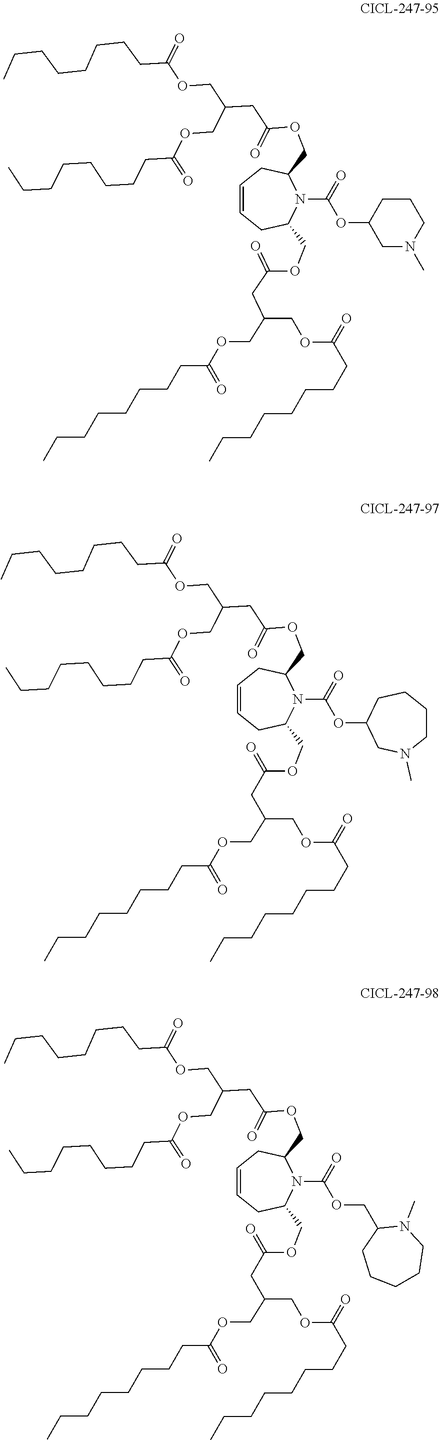

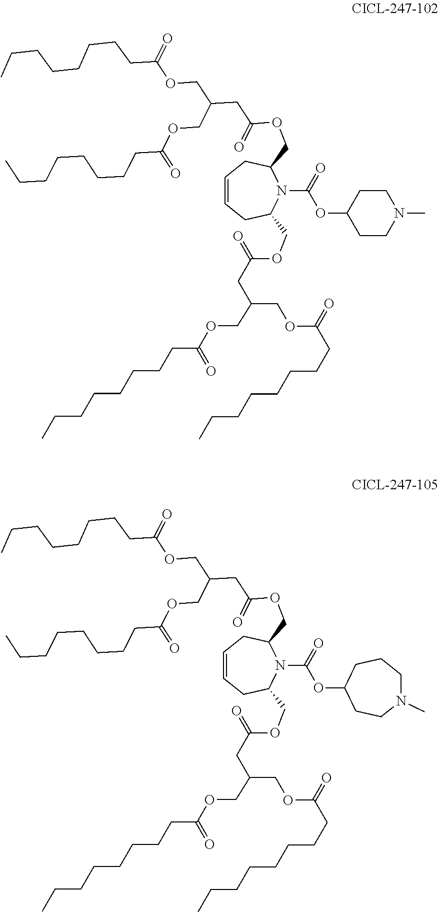

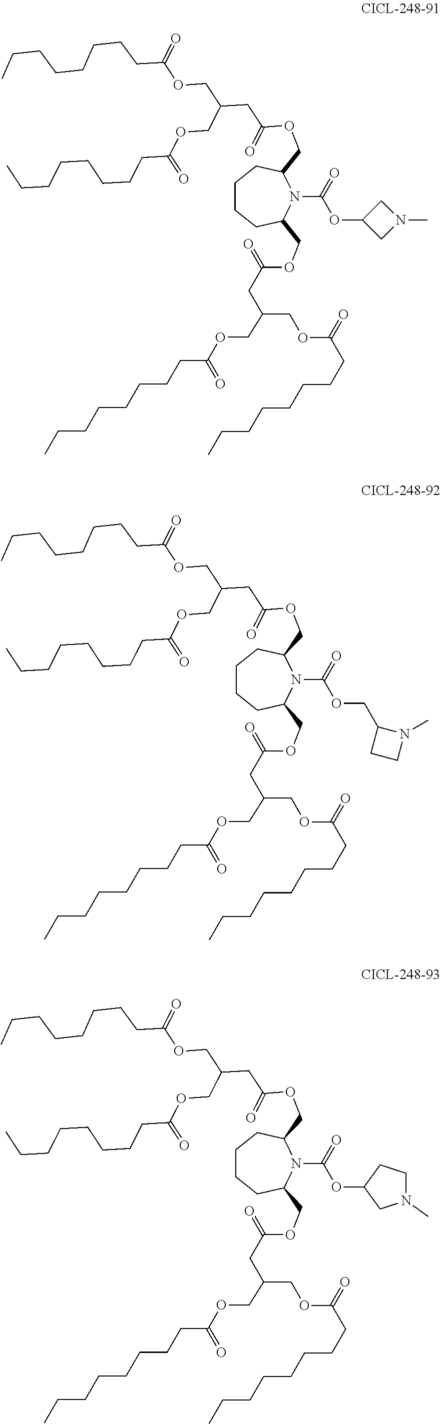

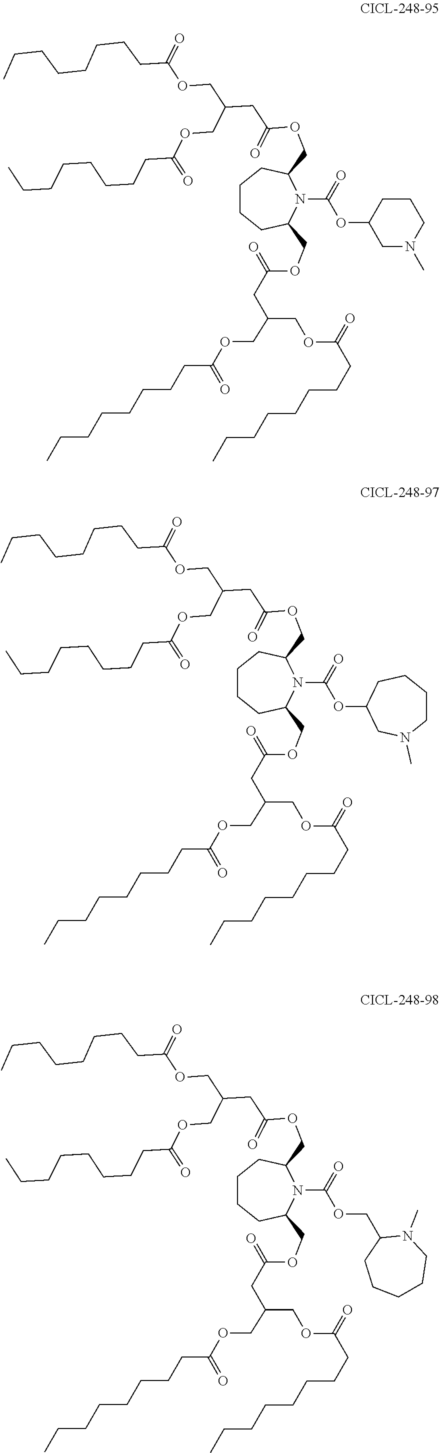

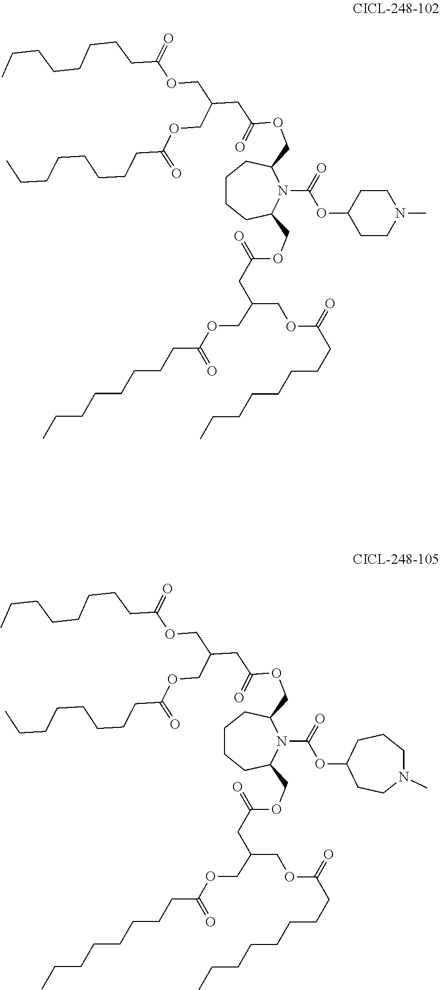

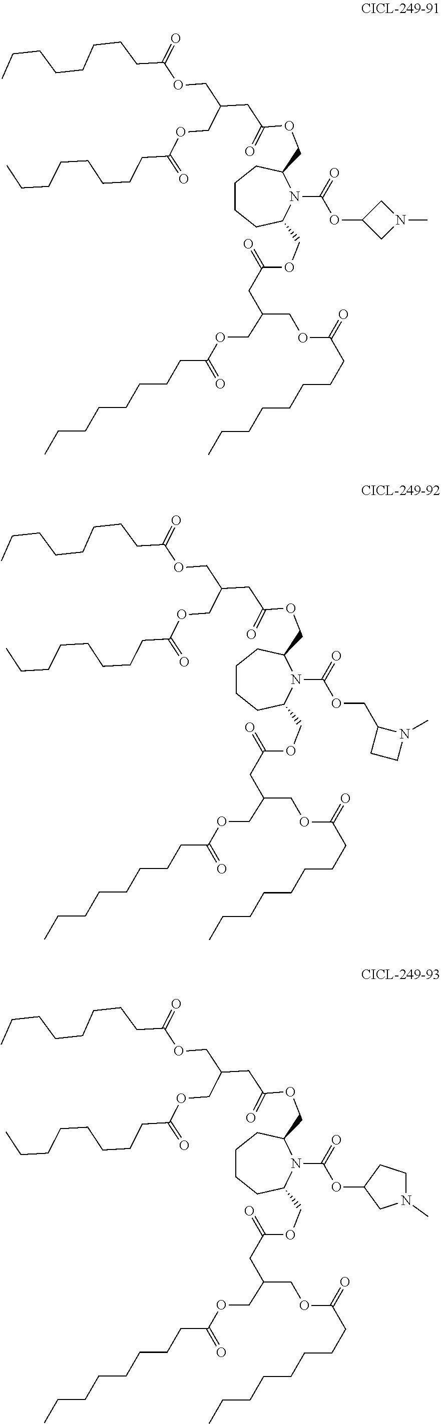

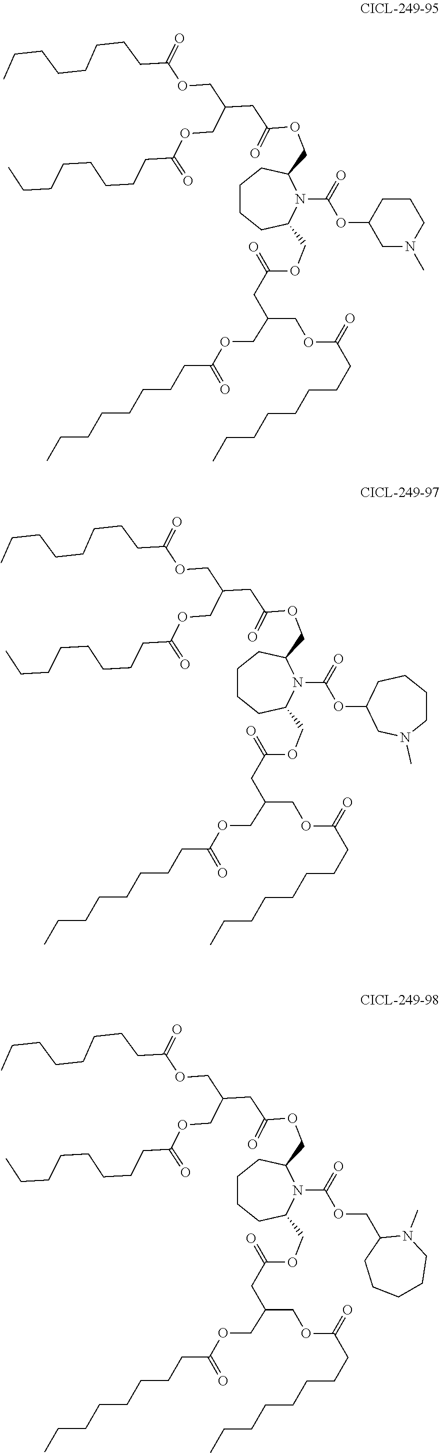

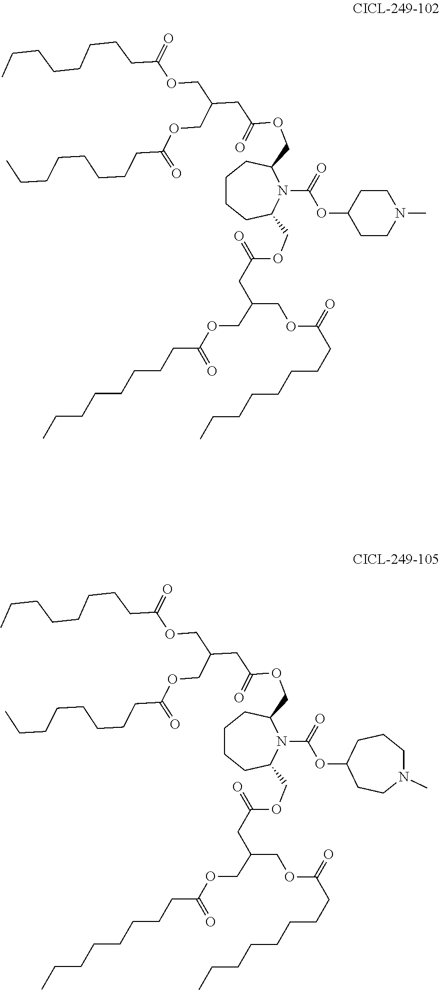

In some embodiments, the ionizable cationic lipid has the structure CICL-207-108:

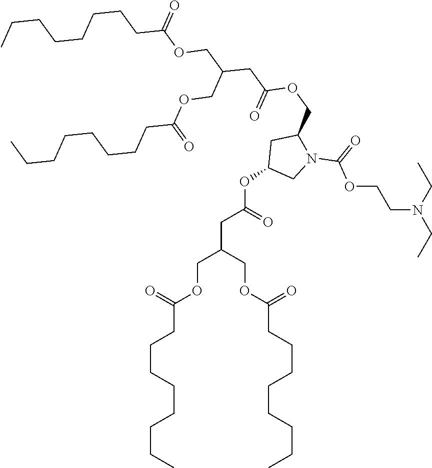 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(CC)CC]

CICL-207-108 is an example of a lipid as disclosed herein in which A¹ and A⁴ are absent, and A², A³, and A⁵ are CHs, and the tail groups are asymmetrically placed relative to the ring nitrogen in a trans configuration. The structure drawn above indicates the absolute stereochemistry for this lipid. Other embodiments include the enantiomer of CICL-207-108 and mixtures of the enantiomers (e.g., a racemate, or other mixtures with various ratios). Further embodiments include or are diastereomers of CICL-207-108, and mixtures thereof.

In some embodiments, the ionizable cationic lipid has the structure CICL-207-109:

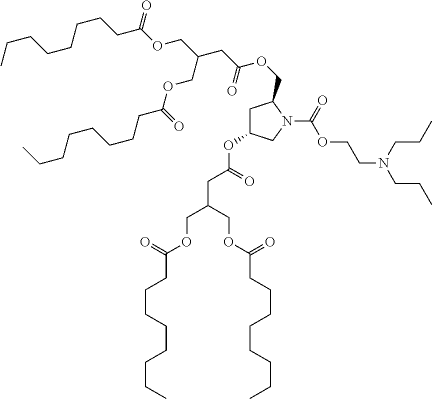 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(CCC)CCC]

CICL-207-109 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-109 and mixtures of the enantiomers (e.g., a racemate, or other mixtures with various ratios). Further embodiments include or are diastereomers of CICL-207-109, and mixtures thereof.

In some embodiments, the ionizable cationic lipid has the structure CICL-207-110.

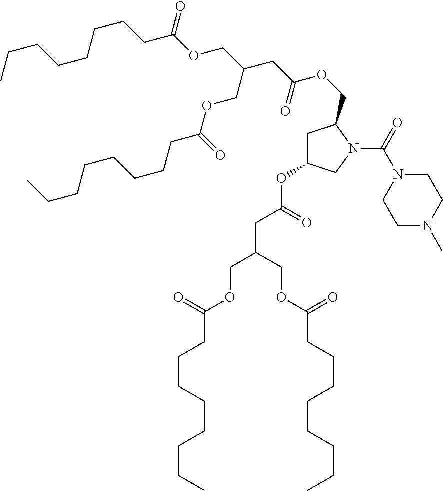 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N1CCN(C)CC1]

CICL-207-110 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-110 and mixtures of the enantiomers (e.g., a racemate, or other mixtures with various ratios). Further embodiments include or are diastereomers of CICL-207-110, and mixtures thereof.

In some embodiments, the ionizable cationic lipid has the structure CICL-207-111:

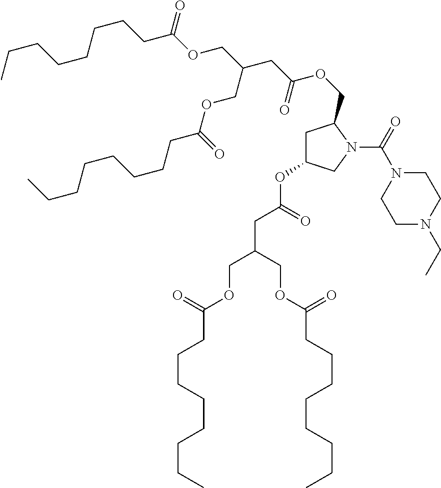 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N1CCN(CC)CC1]

CICL-207-111 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-111 and mixtures of the enantiomers (e.g., a racemate, or other mixtures with various ratios). Further embodiments include or are diastereomers of CICL-207-111, and mixtures thereof.

In some embodiments, the ionizable cationic lipid has the structure CICL-297-112:

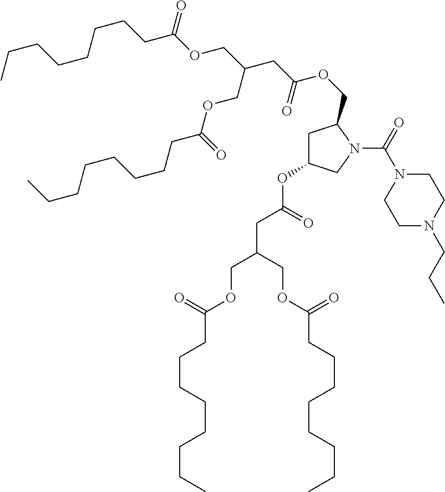 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N1CCN(CCC)CC1]

CICL-207-112 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-112 and mixtures of the enantiomers (e.g., a racemate, or other mixtures with various ratios). Further embodiments include or are diastereomers of CICL-207-112, and mixtures thereof.

In some embodiments, the ionizable cationic lipid has the structure CICL-207-113:

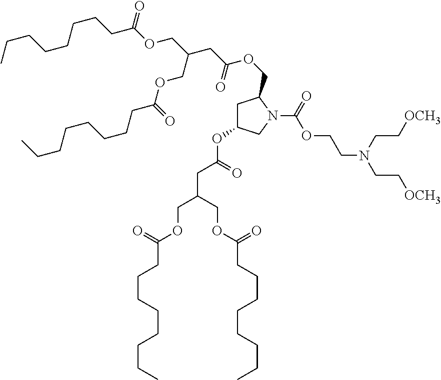 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(CCOC)CCOC]

CICL-207-113 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-113 and mixtures of the enantiomers (e.g., a racemate, or other mixtures with various ratios). Further embodiments include or are diastereomers of CICL-207-113, and mixtures thereof.

In some embodiments, the ionizable cationic lipid has the structure CICL-207-114:

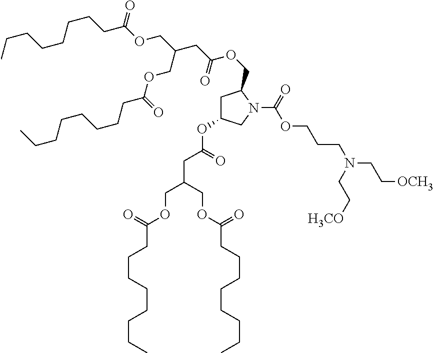 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN(CCOC)CCOC]

CICL-207-114 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-114 and mixtures of the enantiomers (e.g., a racemate, or other mixtures with various ratios). Further embodiments include or are diastereomers of CICL-207-114, and mixtures thereof.

In some embodiments, the ionizable cationic lipid has the structure CICL-207-115:

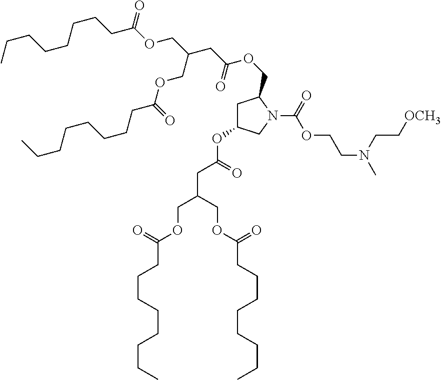 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)CCOC]

CICL-207-115 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-115 and mixtures of the enantiomers (e.g., a racemate, or other mixtures with various ratios). Further embodiments include or are diastereomers of CICL-207-115, and mixtures thereof.

In some embodiments, the ionizable cationic lipid has the structure CICL-207-116:

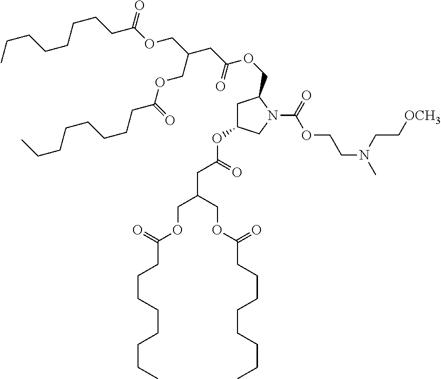 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)CCOC]

CICL-207-116 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-116 and mixtures of the enantiomers (e.g., a racemate, or other mixtures with various ratios). Further embodiments include or are diastereomers of CICL-207-116, and mixtures thereof.

In some embodiments, the ionizable cationic lipid has the structure CICL-207-117:

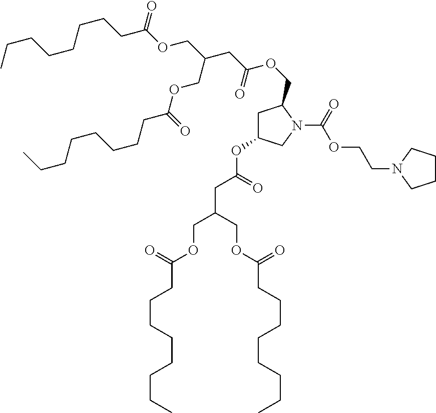 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCCC1]

CICL-207-117 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-117 and mixtures of the enantiomers (e.g., a racemate, or other mixtures with various ratios). Further embodiments include or are diastereomers of CICL-207-117, and mixtures thereof.

In some embodiments, the ionizable cationic lipid has the structure CICL-207-118:

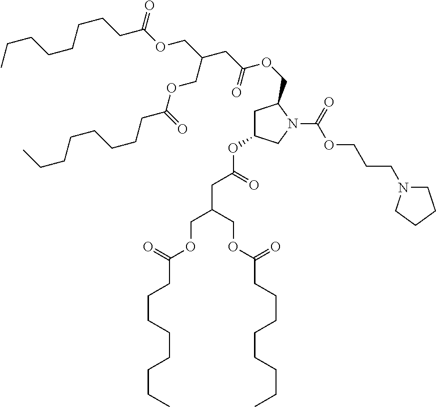 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCCC1]

CICL-207-118 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-118 and mixtures of the enantiomers (e.g., a racemate, or other mixtures with various ratios). Further embodiments include or are diastereomers of CICL-207-118, and mixtures thereof.

In some embodiments, the ionizable cationic lipid has the structure CICL-207-119:

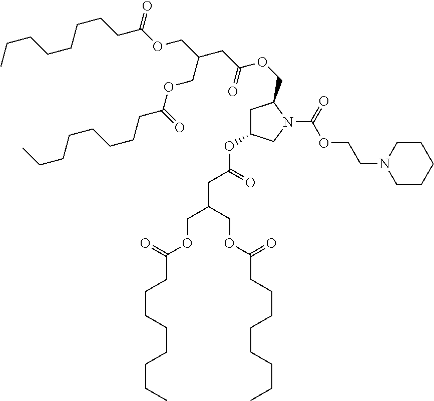 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCCCC1]

CICL-207-119 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-119 and mixtures of the enantiomers (e.g., a racemate, or other mixtures with various ratios). Further embodiments include or are diastereomers of CICL-207-119, and mixtures thereof.

In some embodiments, the ionizable cationic lipid has the structure CICL-207-120:

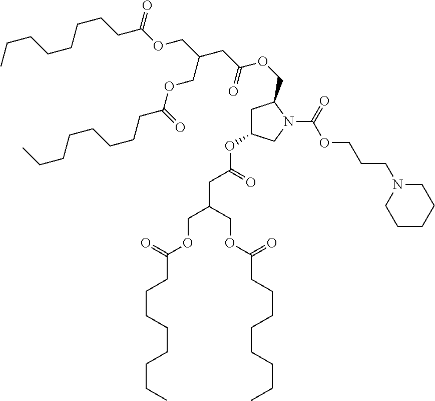 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCCCC1]

CICL-207-120 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-120 and mixtures of the enantiomers (e.g., a racemate, or other mixtures with various ratios). Further embodiments include or are diastereomers of CICL-207-120, and mixtures thereof.

In some embodiments, the ionizable cationic lipid has the structure CICL-207-121:

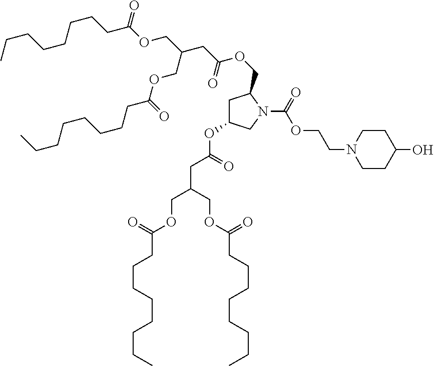 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCC(O)CC1]

CICL-207-121 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-121 and mixtures of the enantiomers (e.g., a racemate, or other mixtures with various ratios). Further embodiments include or are diastereomers of CICL-207-121, and mixtures thereof.

In some embodiments, the ionizable cationic lipid has the structure CICL-207-122:

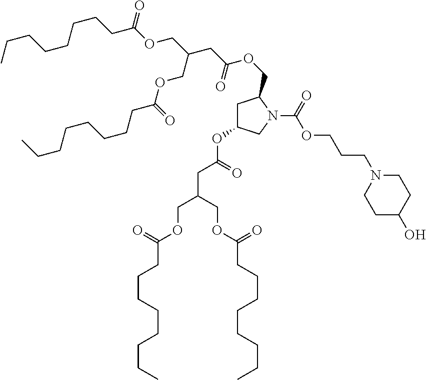 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCC(O)CC1]

CICL-207-122 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-122 and mixtures of the enantiomers (e.g., a racemate, or other mixtures with various ratios). Further embodiments include or are diastereomers of CICL-207-122, and mixtures thereof.

In some embodiments, the ionizable cationic lipid has the structure CICL-207-123:

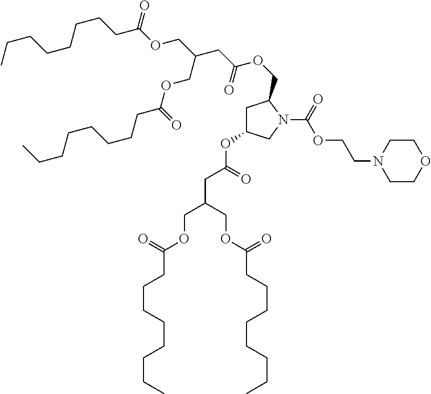 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCOCC1]

CICL-207-123 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-123 and mixtures of the enantiomers (e.g., a racemate, or other mixtures with various ratios). Further embodiments include or are diastereomers of CICL-207-123, and mixtures thereof.

In some embodiments, the ionizable cationic lipid has the structure CICL-207-124:

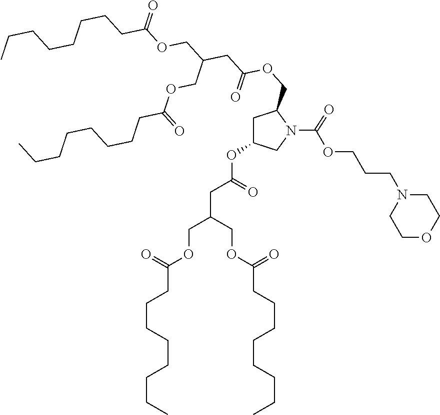 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCOCC1]

CICL-207-124 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-124 and mixtures of the enantiomers (e.g., a racemate, or other mixtures with various ratios). Further embodiments include or are diastereomers of CICL-207-124, and mixtures thereof.

In some embodiments, the ionizable cationic lipid has the structure CICL-207-125:

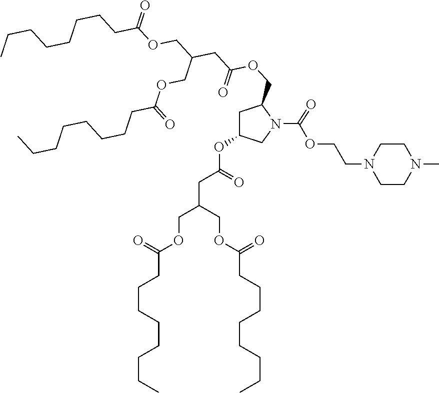 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCN(C)CC1]

CICL-207-125 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-125 and mixtures of the enantiomers (e.g., a racemate, or other mixtures with various ratios). Further embodiments include or are diastereomers of CICL-207-125, and mixtures thereof.

In some embodiments, the ionizable cationic lipid has the structure CICL-207-126:

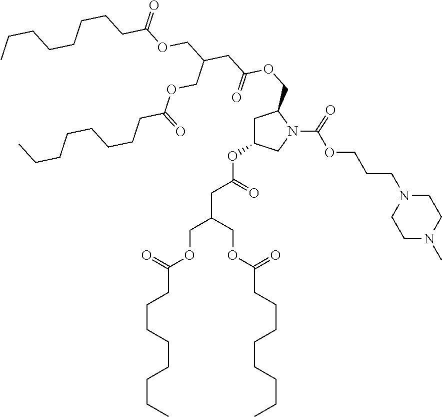 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCN(C)CC1]

CICL-207-126 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-126 and mixtures of the enantiomers (e.g., a racemate, or other mixtures with various ratios). Further embodiments include or are diastereomers of CICL-207-126, and mixtures thereof.

In some embodiments, the ionizable cationic lipid has the structure CICL-207-127:

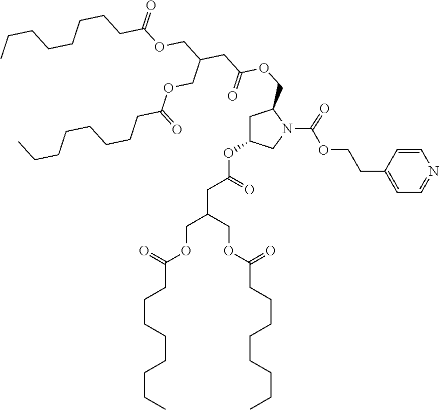 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCc1ccncc1]

CICL-207-127 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-127 and mixtures of the enantiomers (e.g., a racemate, or other mixtures with various ratios). Further embodiments include or are diastereomers of CICL-207-127, and mixtures thereof.

In some embodiments, the ionizable cationic lipid has the structure CICL-207-128:

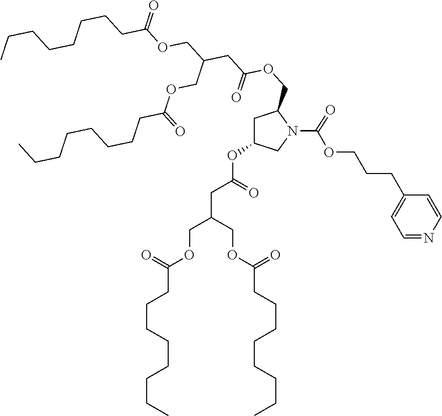 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCc1ccncc1]

CICL-207-128 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-128 and mixtures of the enantiomers (e.g., a racemate, or other mixtures with various ratios). Further embodiments include or are diastereomers of CICL-207-128, and mixtures thereof.

In some embodiments, the ionizable cationic lipid has the structure CICL-207-129:

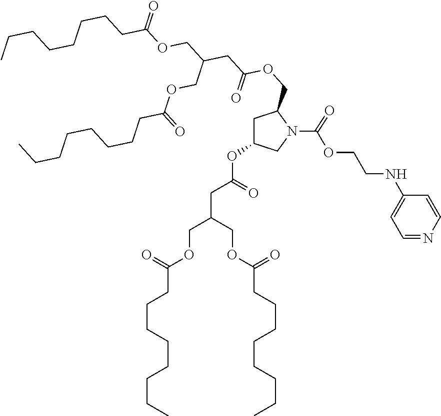 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCNc1ccncc1]

CICL-207-129 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-129 and mixtures of the enantiomers (e.g., a racemate, or other mixtures with various ratios). Further embodiments include or are diastereomers of CICL-207-129, and mixtures thereof.

In some embodiments, the ionizable cationic lipid has the structure CICL-207-130:

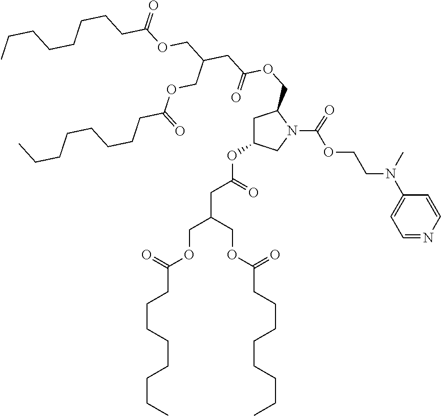 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)c1ccncc1]

CICL-207-130 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-130 and mixtures of the enantiomers (e.g., a racemate, or other mixtures with various ratios). Further embodiments include or are diastereomers of CICL-207-130, and mixtures thereof.

In some embodiments, the ionizable cationic lipid has the structure CICL-207-131:

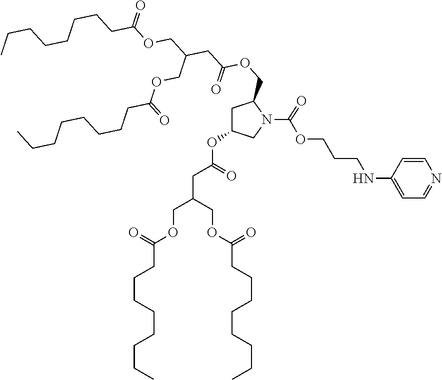 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCNc1ccncc1]

CICL-207-131 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-131 and mixtures of the enantiomers (e.g., a racemate, or other mixtures with various ratios). Further embodiments include or are diastereomers of CICL-207-131, and mixtures thereof.

In some embodiments, the ionizable cationic lipid has the structure CICL-207-132:

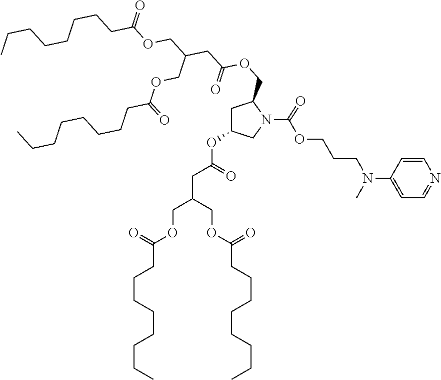 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN(C)c1ccncc1]

CICL-207-132 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-132 and mixtures of the enantiomers (e.g., a racemate, or other mixtures with various ratios). Further embodiments include or are diastereomers of CICL-207-132, and mixtures thereof.

In some embodiments, the ionizable cationic lipid has the structure CICL-207-133:

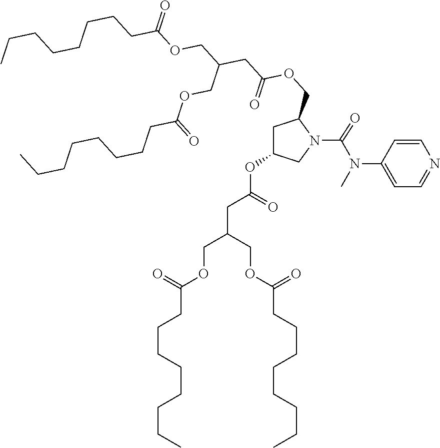 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N(C)c1ccncc1]

CICL-207-133 is an example of a lipid as disclosed herein in which A¹ and A⁴ are absent, and A², A³, and A⁵ are CH₂, and the tail groups are asymmetrically placed relative to the ring nitrogen in a trans configuration. The structure drawn above indicates the absolute stereochemistry for this lipid. Other embodiments include the enantiomer of CICL-207-133 and mixtures of the enantiomers (e.g., a racemate, or other mixtures with various ratios). Further embodiments include or are diastereomers of CICL-207-133, and mixtures thereof.

In some embodiments, the ionizable cationic lipid has a structure selected from the following:

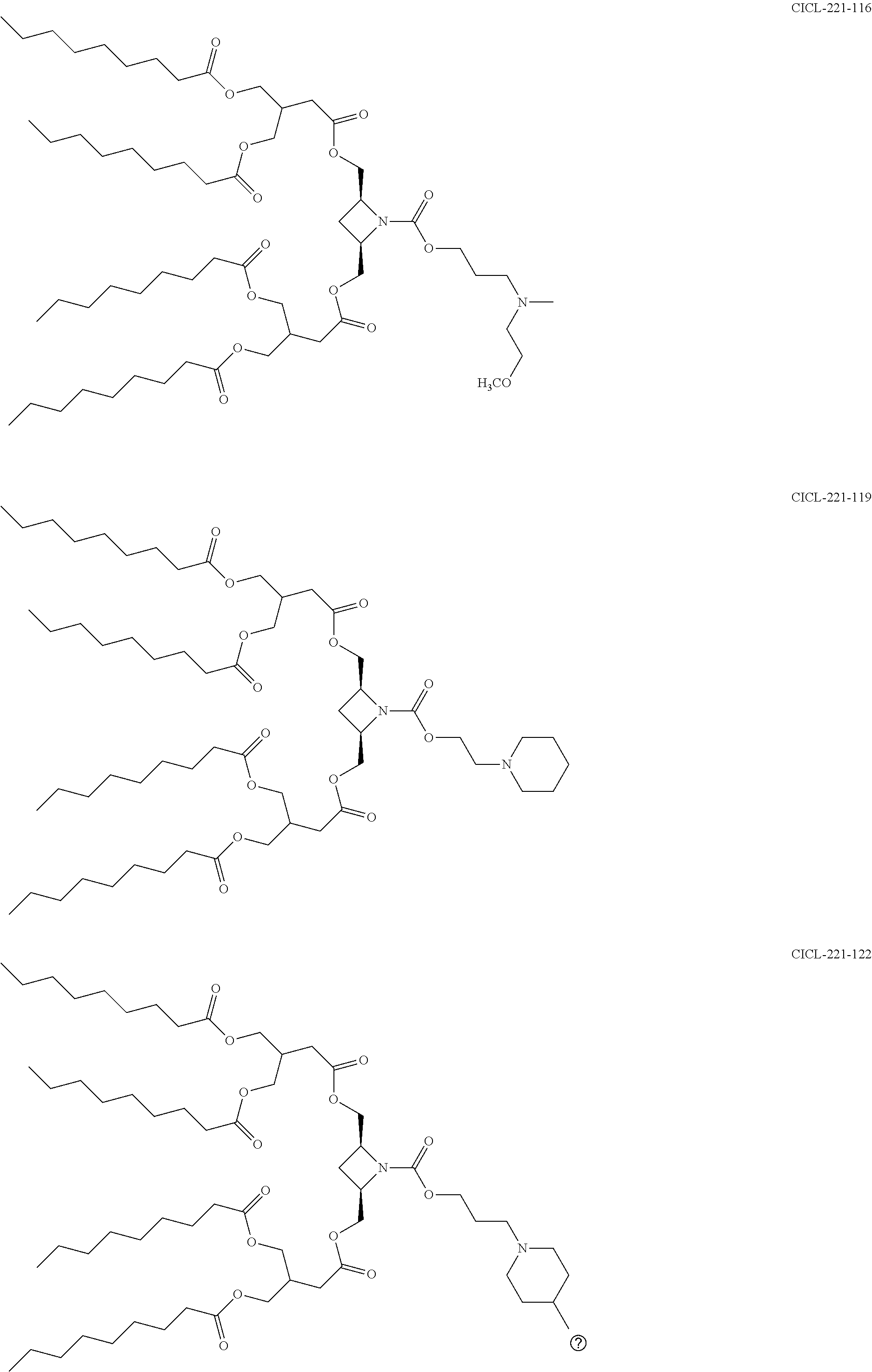

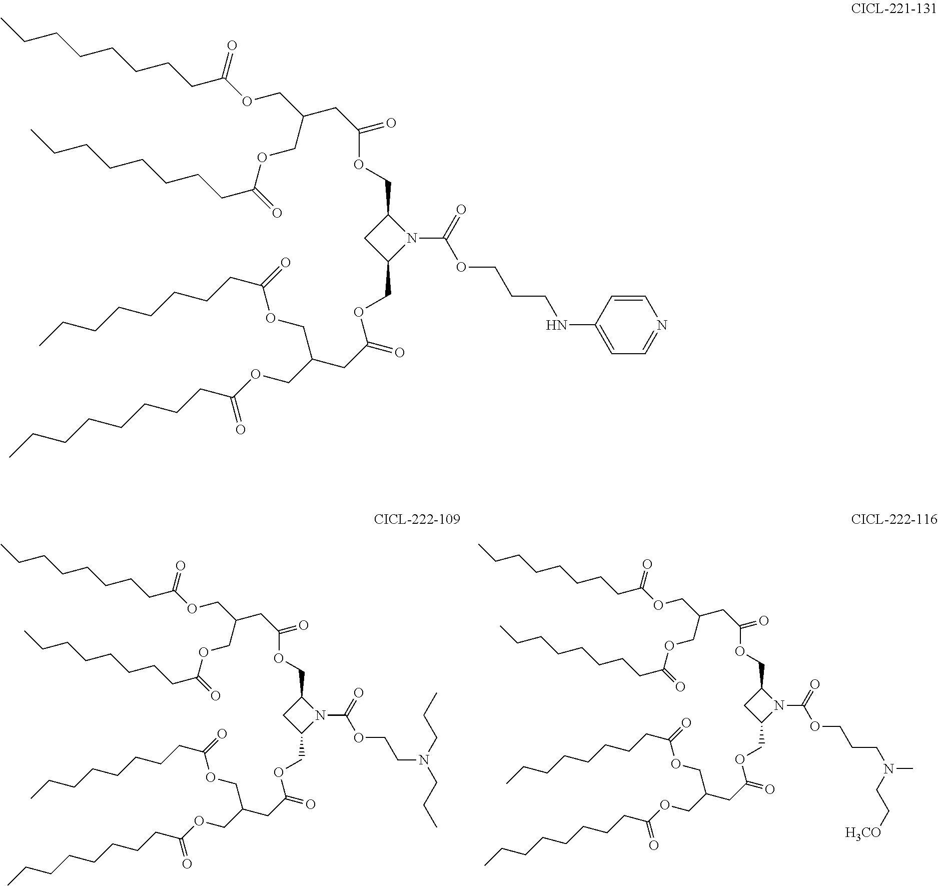 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OCCCNc1ccncc1 ; CCCCCCCCC(=O)OCC(COC(=O)CCCCCCCC)CC(=O)OC[C@@H]1C[C@@H](COC(=O)CC(COC(=O)CCCCCCCC)COC(=O)CCCCCCCC)N1C(=O)OCCN(CCC)CCC ; CCCCCCCCC(=O)OCC(COC(=O)CCCCCCCC)CC(=O)OC[C@@H]1C[C@@H](COC(=O)CC(COC(=O)CCCCCCCC)COC(=O)CCCCCCCC)N1C(=O)OCCCN(C)CCOC]

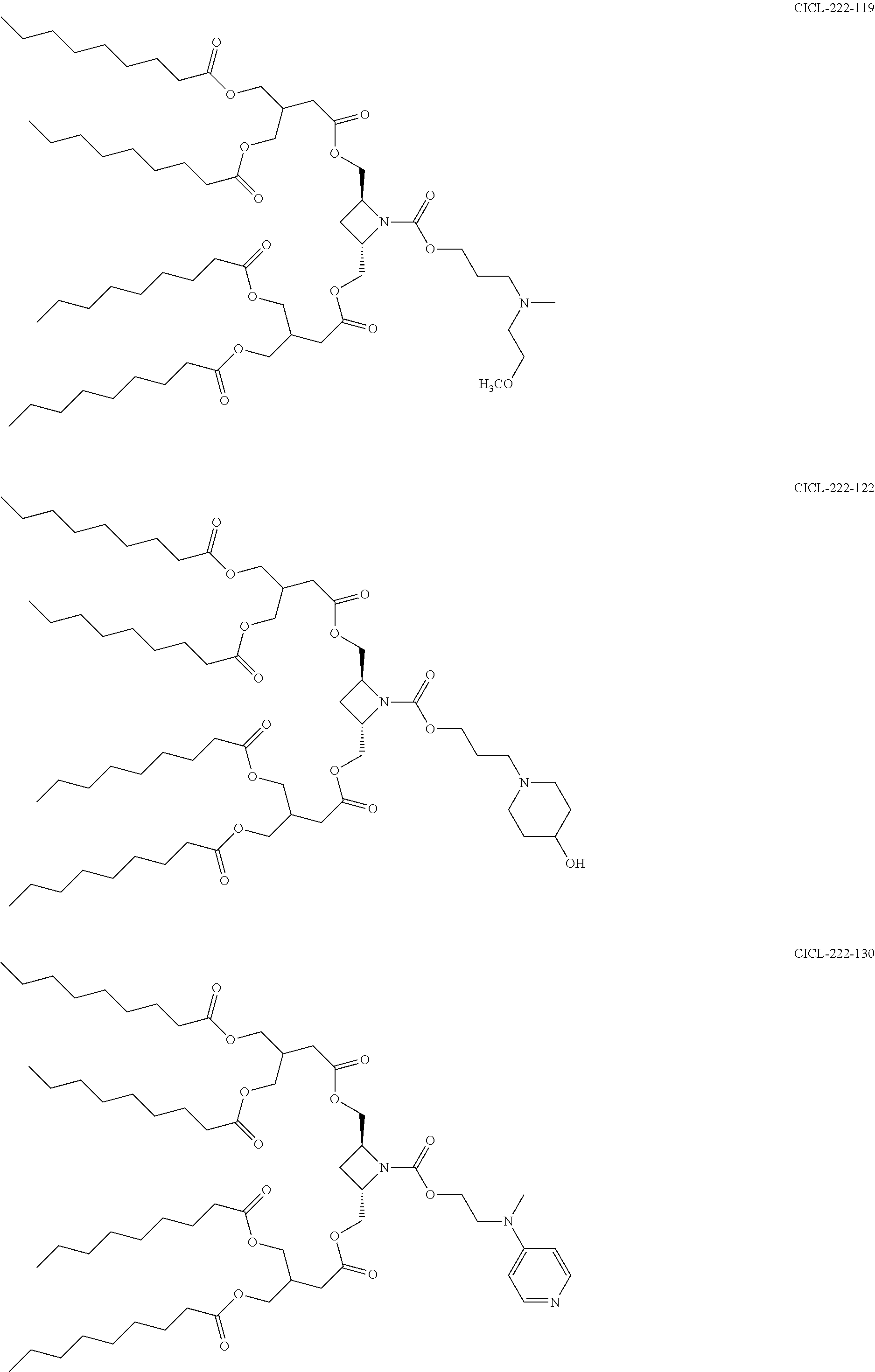

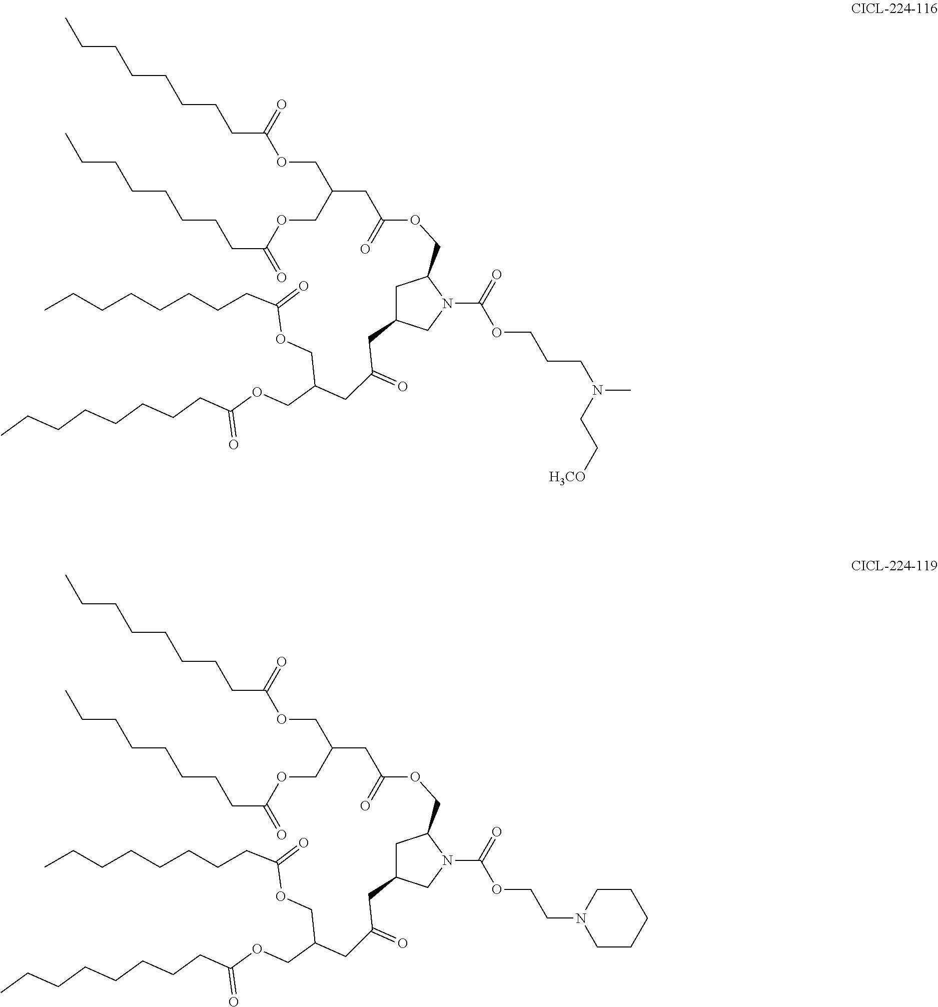

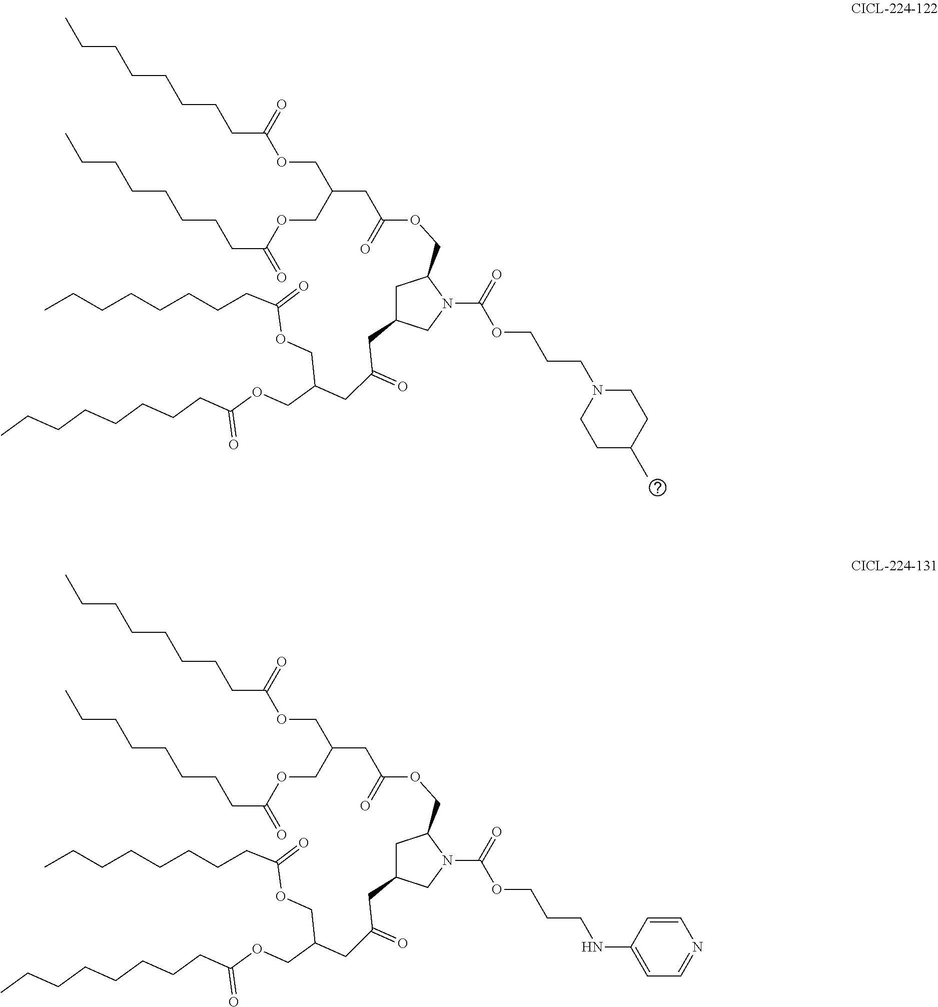

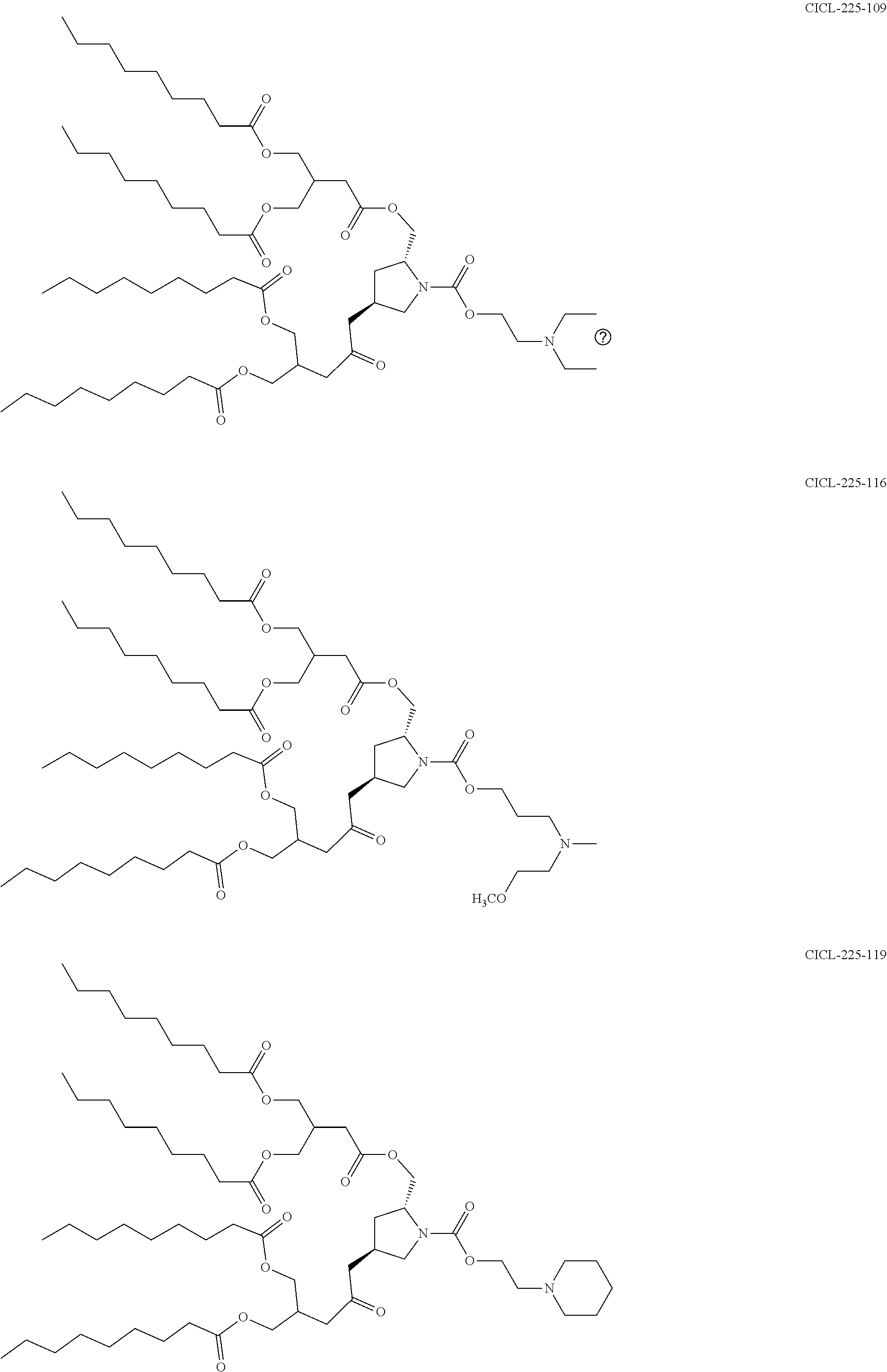

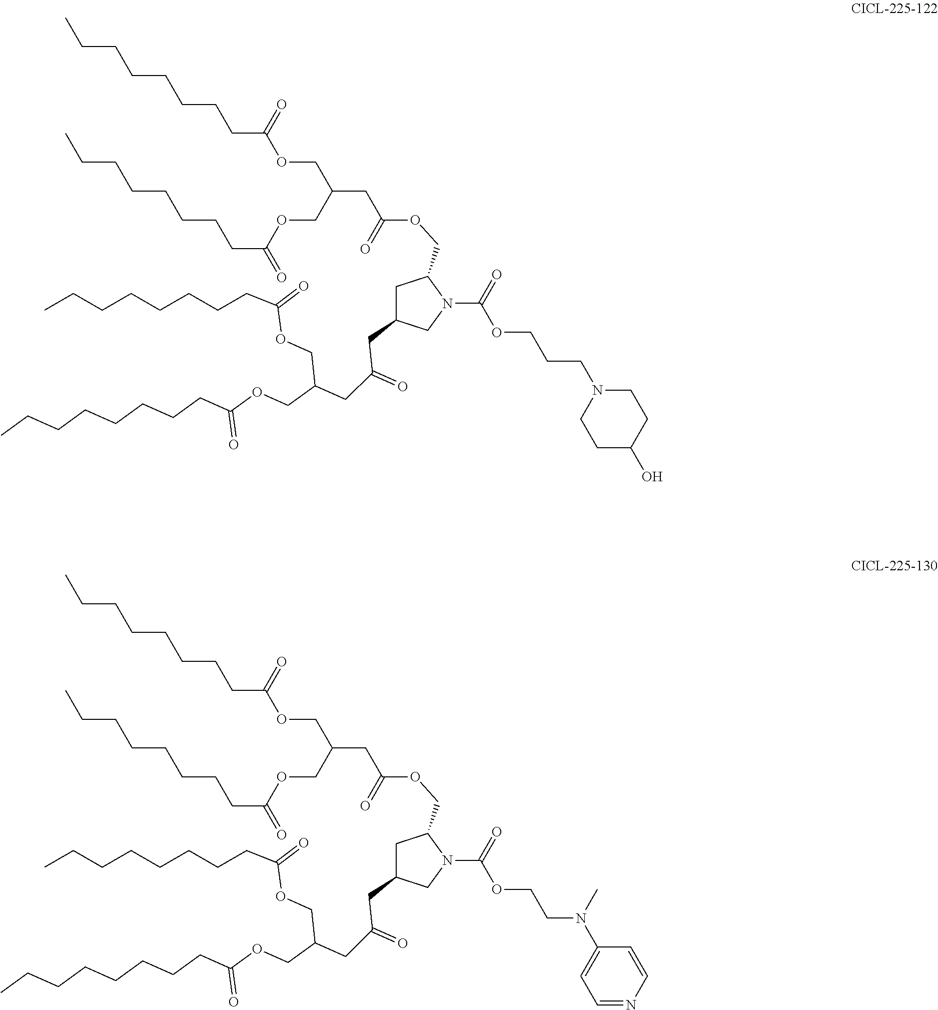 [SMILES: CCCCCCCCC(=O)OCC(COC(=O)CCCCCCCC)CC(=O)C[C@H]1C[C@H](COC(=O)CC(COC(=O)CCCCCCCC)COC(=O)CCCCCCCC)N(C(=O)OCCCN2CCC(O)CC2)C1 ; CCCCCCCCC(=O)OCC(COC(=O)CCCCCCCC)CC(=O)C[C@H]1C[C@H](COC(=O)CC(COC(=O)CCCCCCCC)COC(=O)CCCCCCCC)N(C(=O)OCCN(C)c2ccncc2)C1]

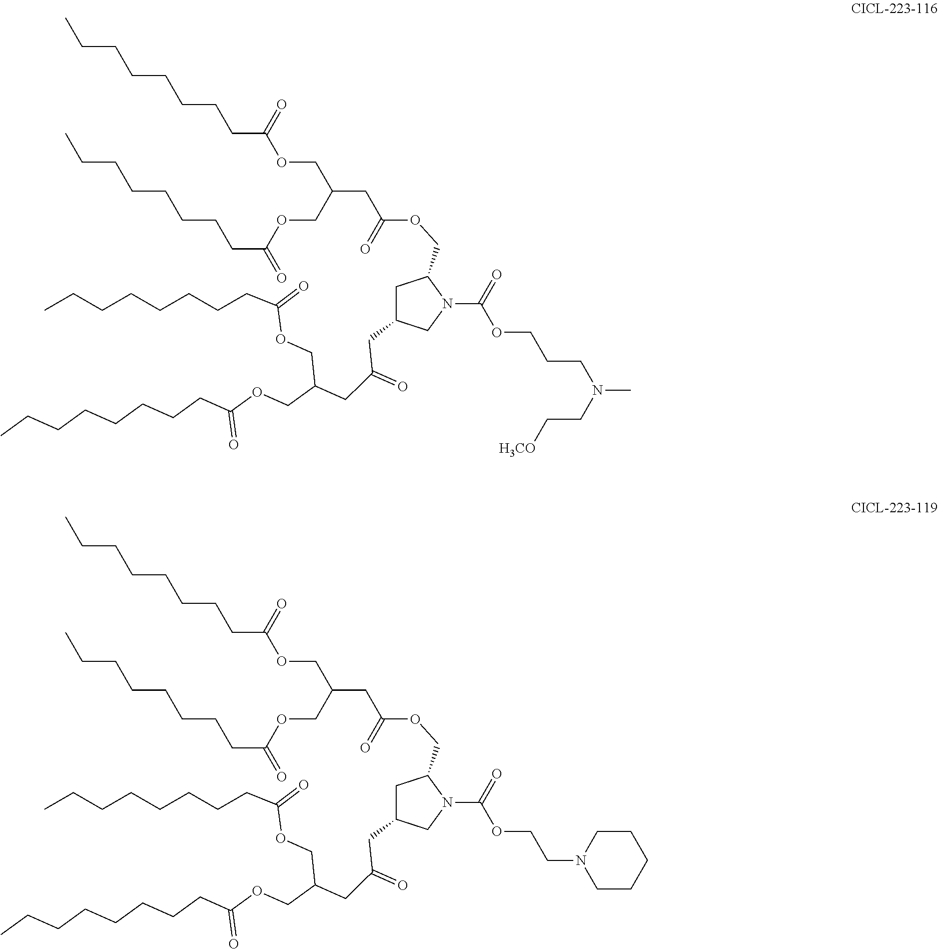

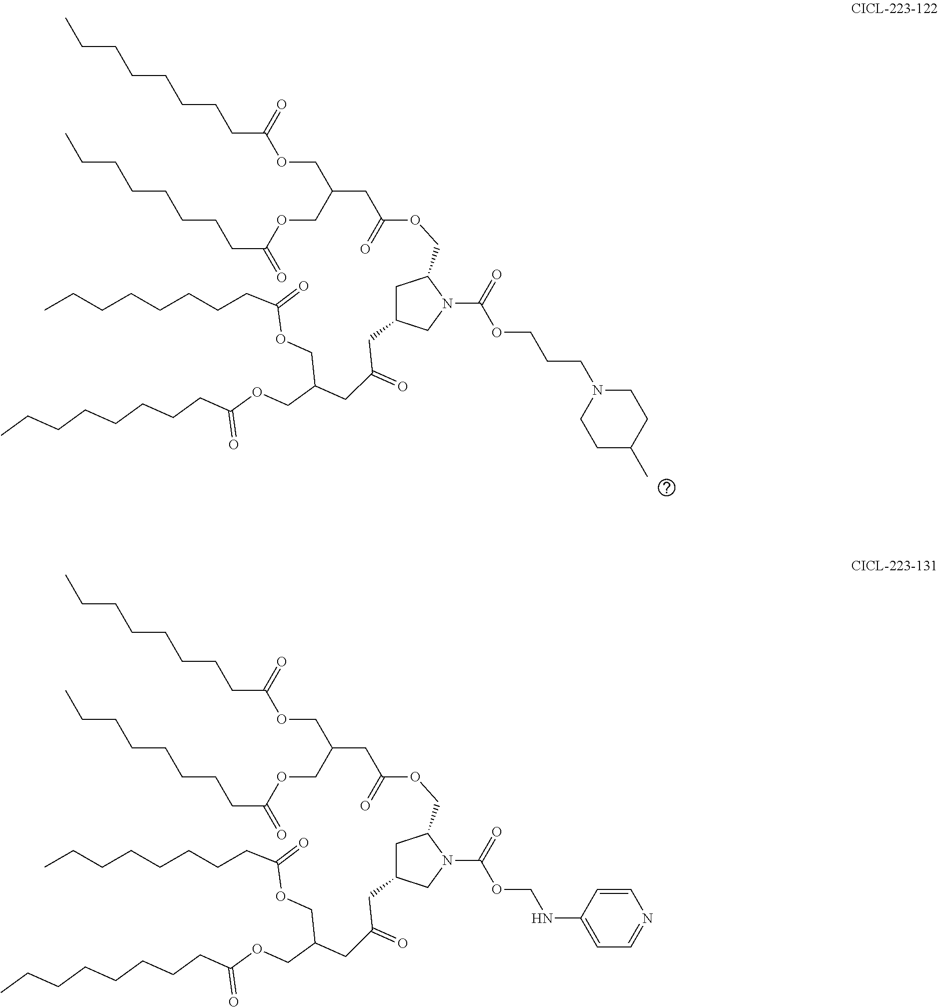

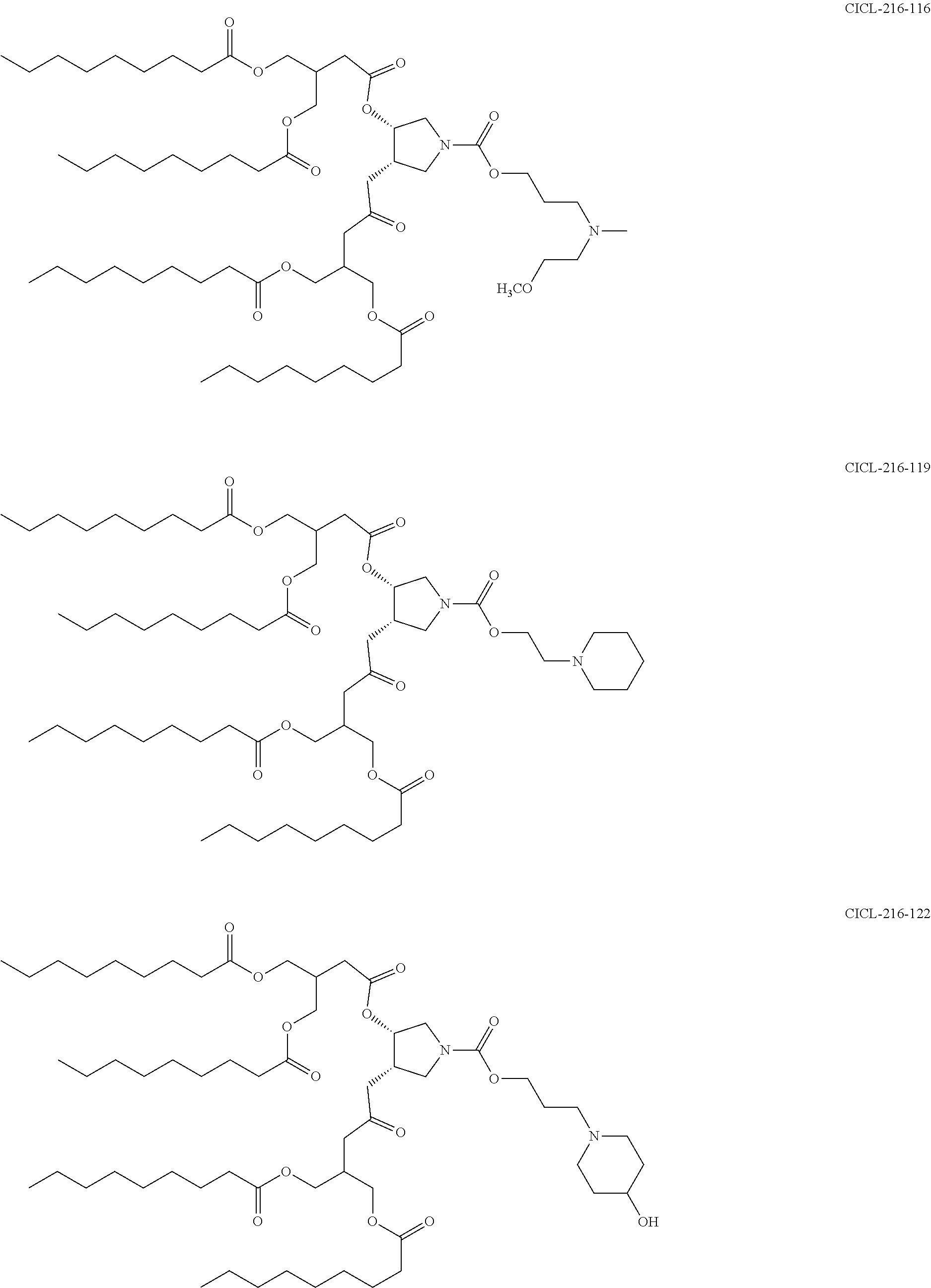

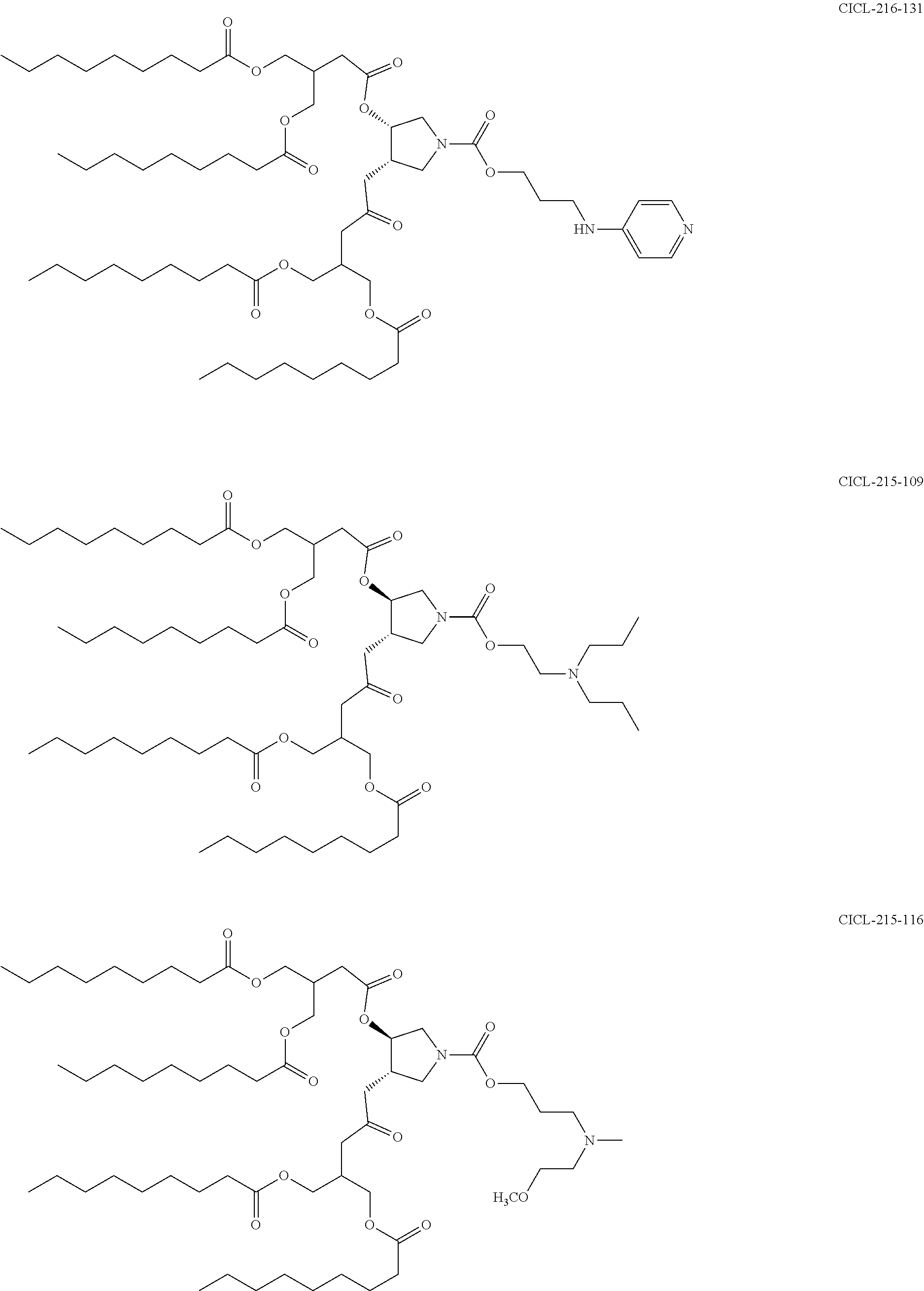

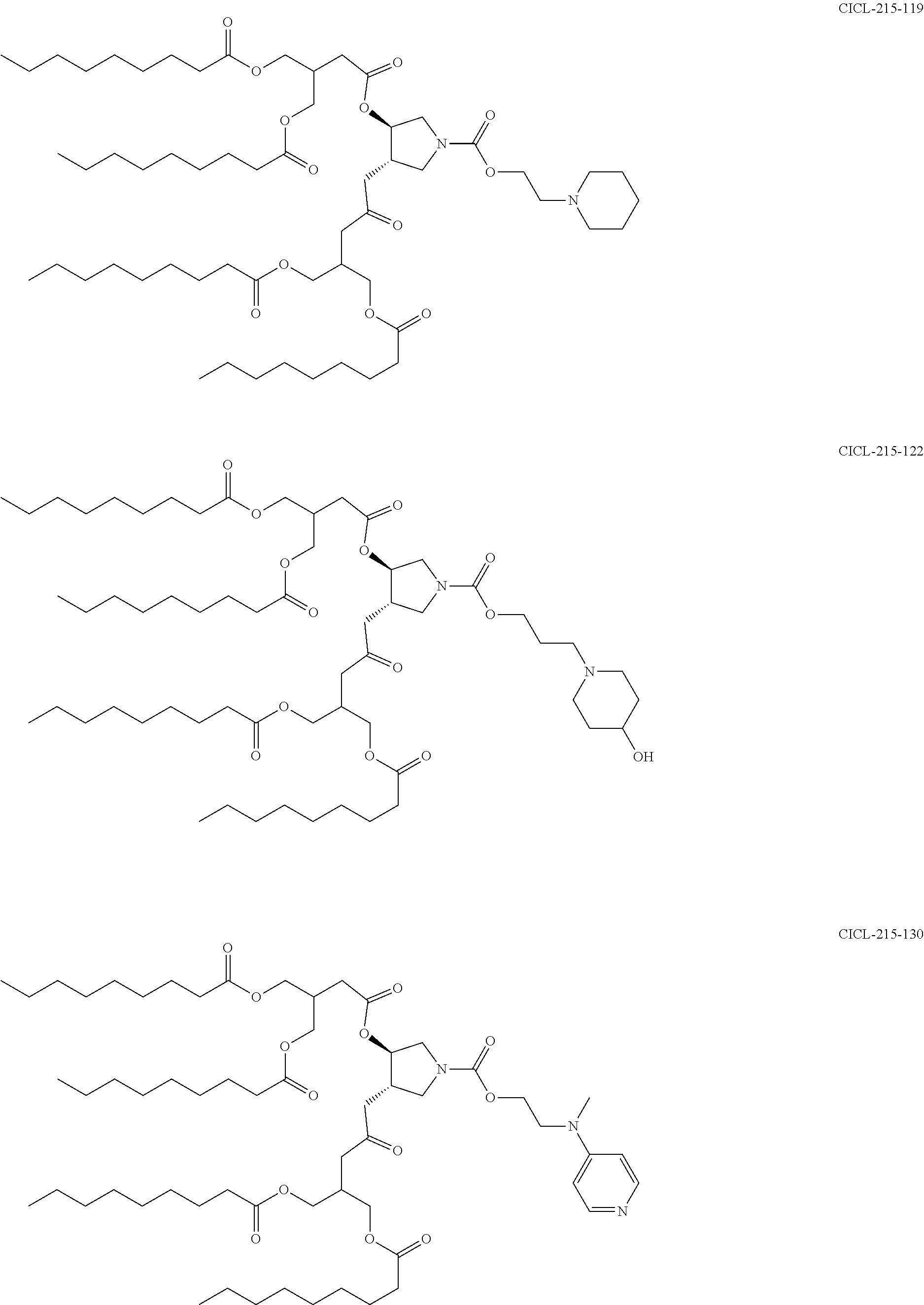 [SMILES: CCCCCCCCC(=O)OCC(COC(=O)CCCCCCCC)CC(=O)C[C@H]1CN(C(=O)OCCN2CCCCC2)C[C@@H]1OC(=O)CC(COC(=O)CCCCCCCC)COC(=O)CCCCCCCC ; CCCCCCCCC(=O)OCC(COC(=O)CCCCCCCC)CC(=O)C[C@H]1CN(C(=O)OCCCN2CCC(O)CC2)C[C@@H]1OC(=O)CC(COC(=O)CCCCCCCC)COC(=O)CCCCCCCC ; CCCCCCCCC(=O)OCC(COC(=O)CCCCCCCC)CC(=O)C[C@H]1CN(C(=O)OCCN(C)c2ccncc2)C[C@@H]1OC(=O)CC(COC(=O)CCCCCCCC)COC(=O)CCCCCCCC]

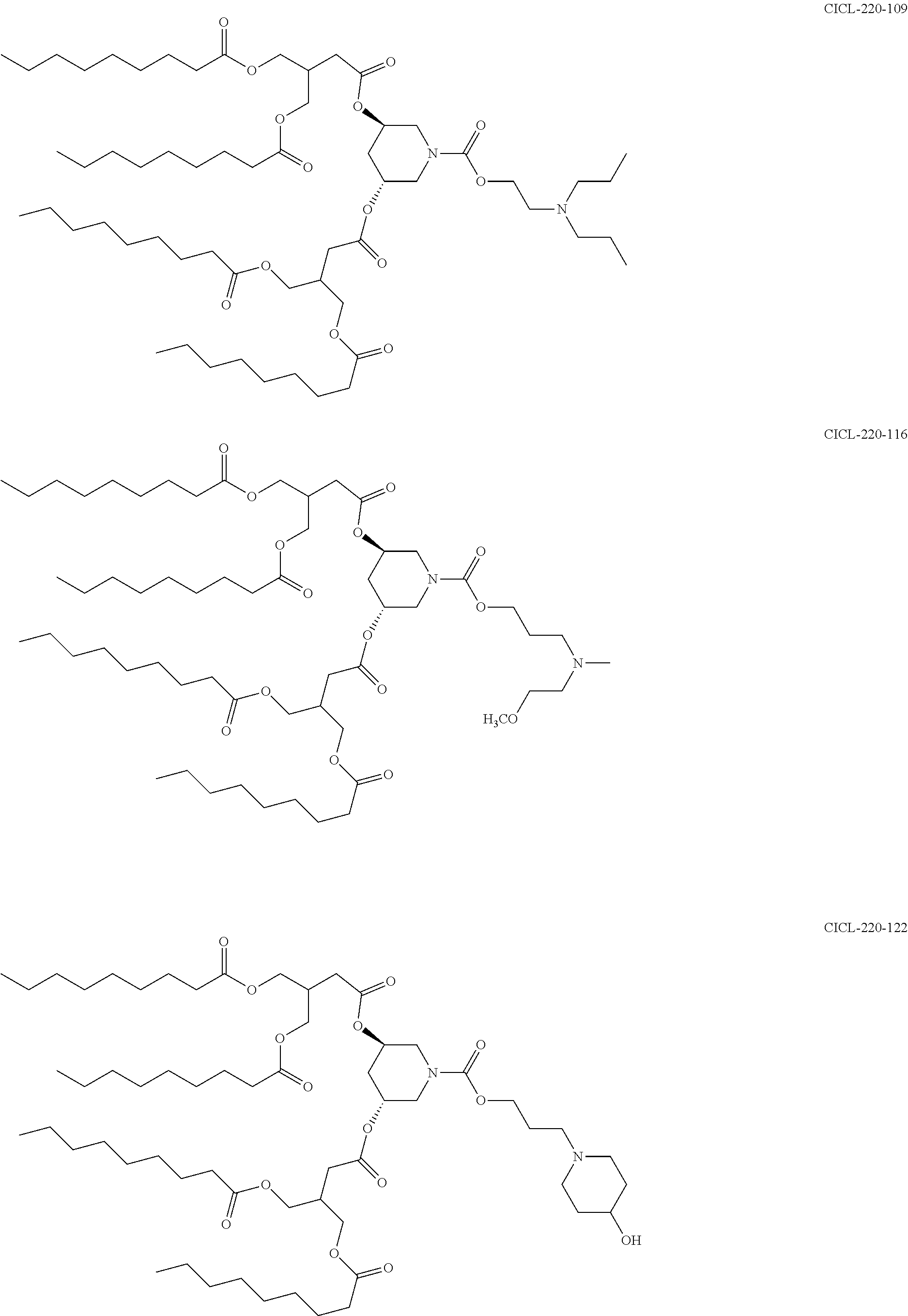

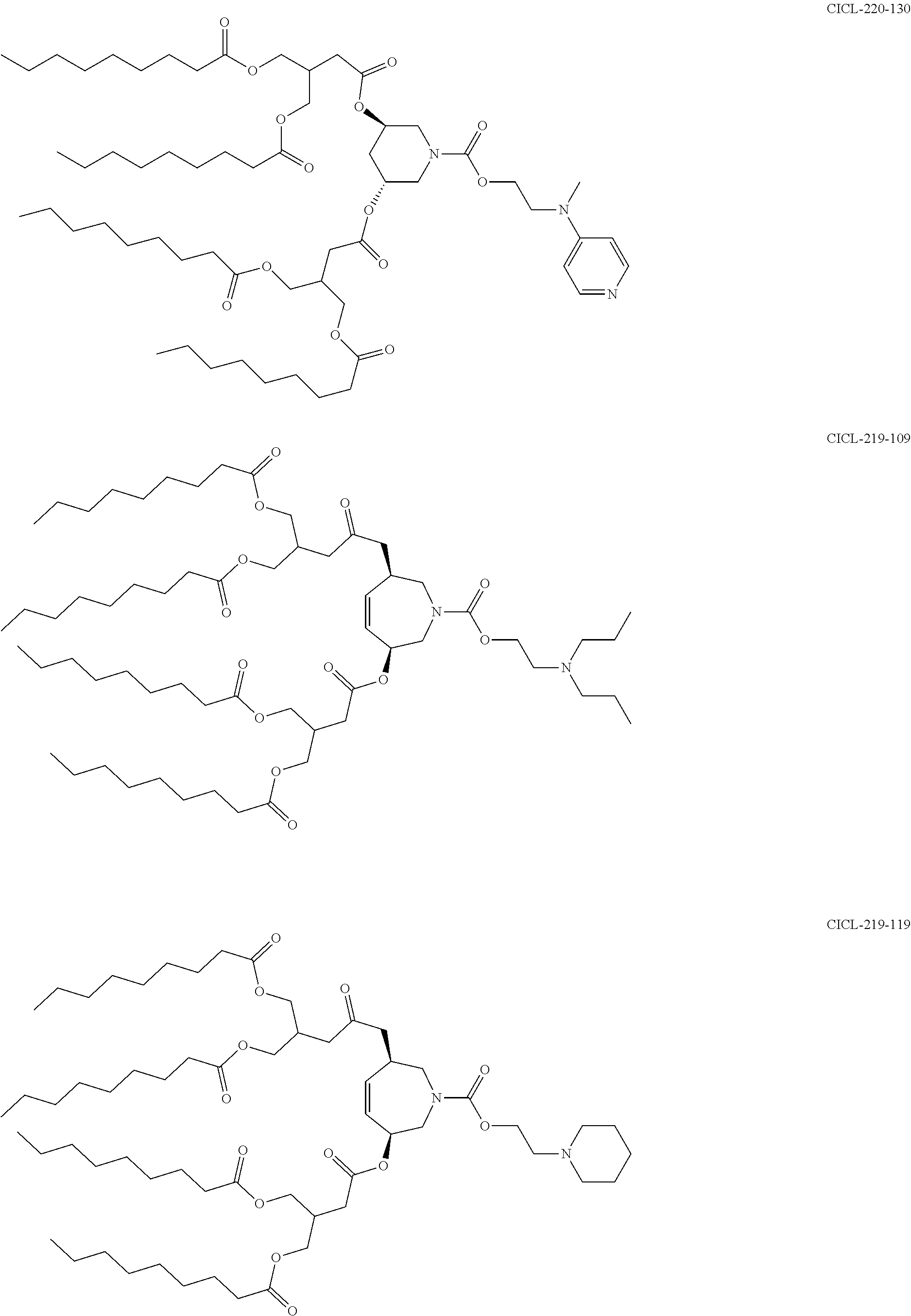

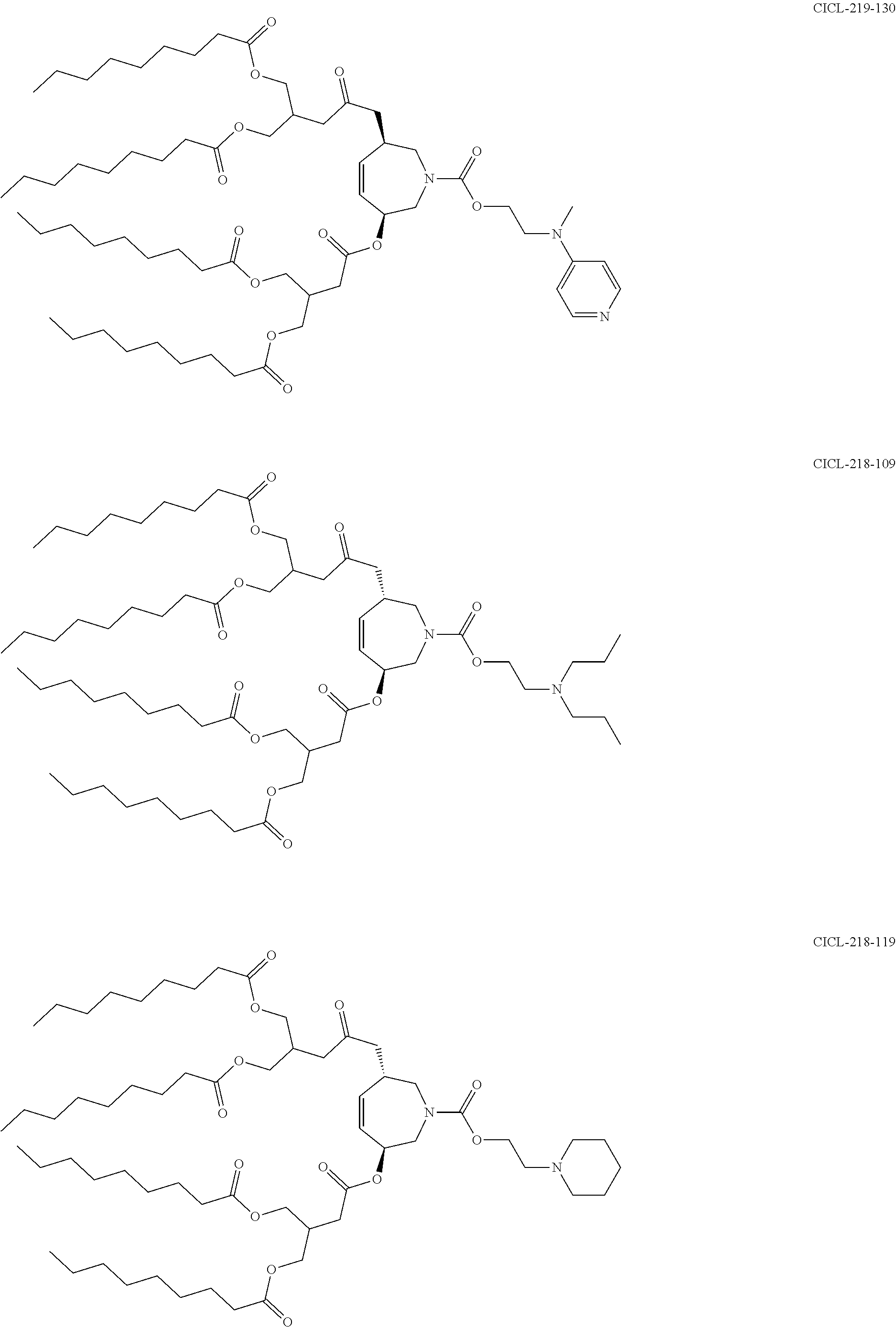

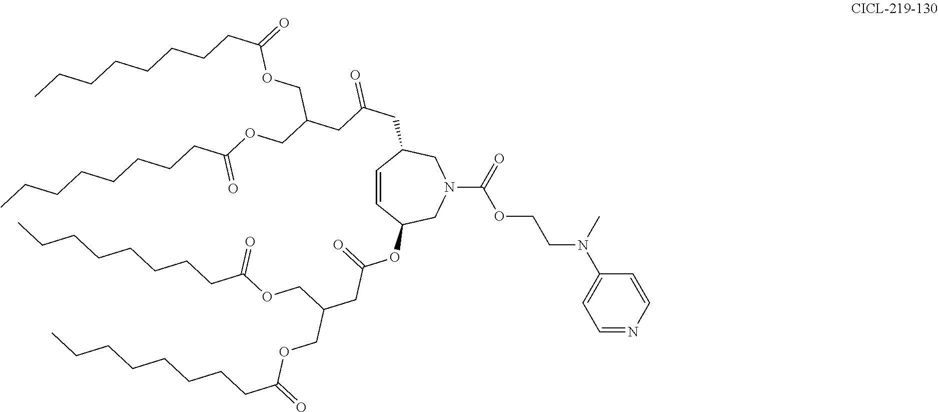 [SMILES: CCCCCCCCC(=O)OCC(COC(=O)CCCCCCCC)CC(=O)C[C@H]1C=C[C@H](OC(=O)CC(COC(=O)CCCCCCCC)COC(=O)CCCCCCCC)CN(C(=O)OCCN(C)c2ccncc2)C1]

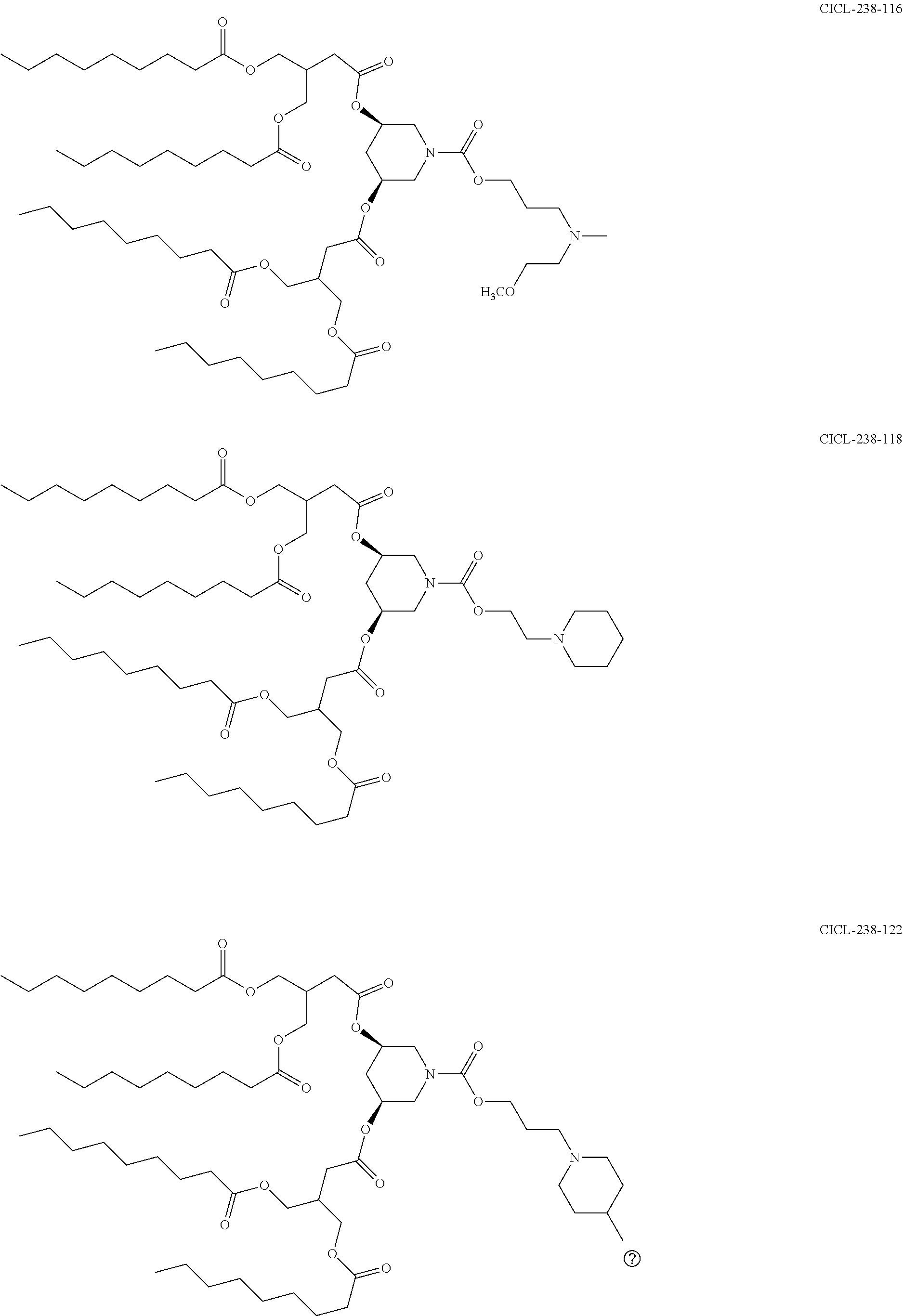

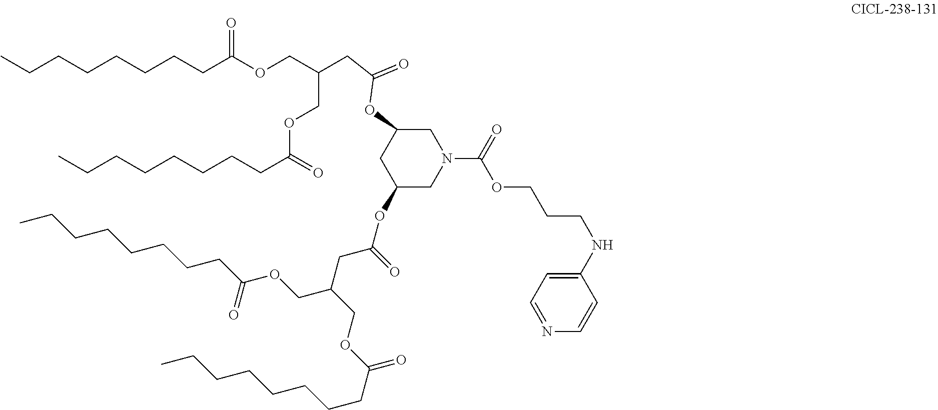 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OCCCNc2ccncc2)C1]

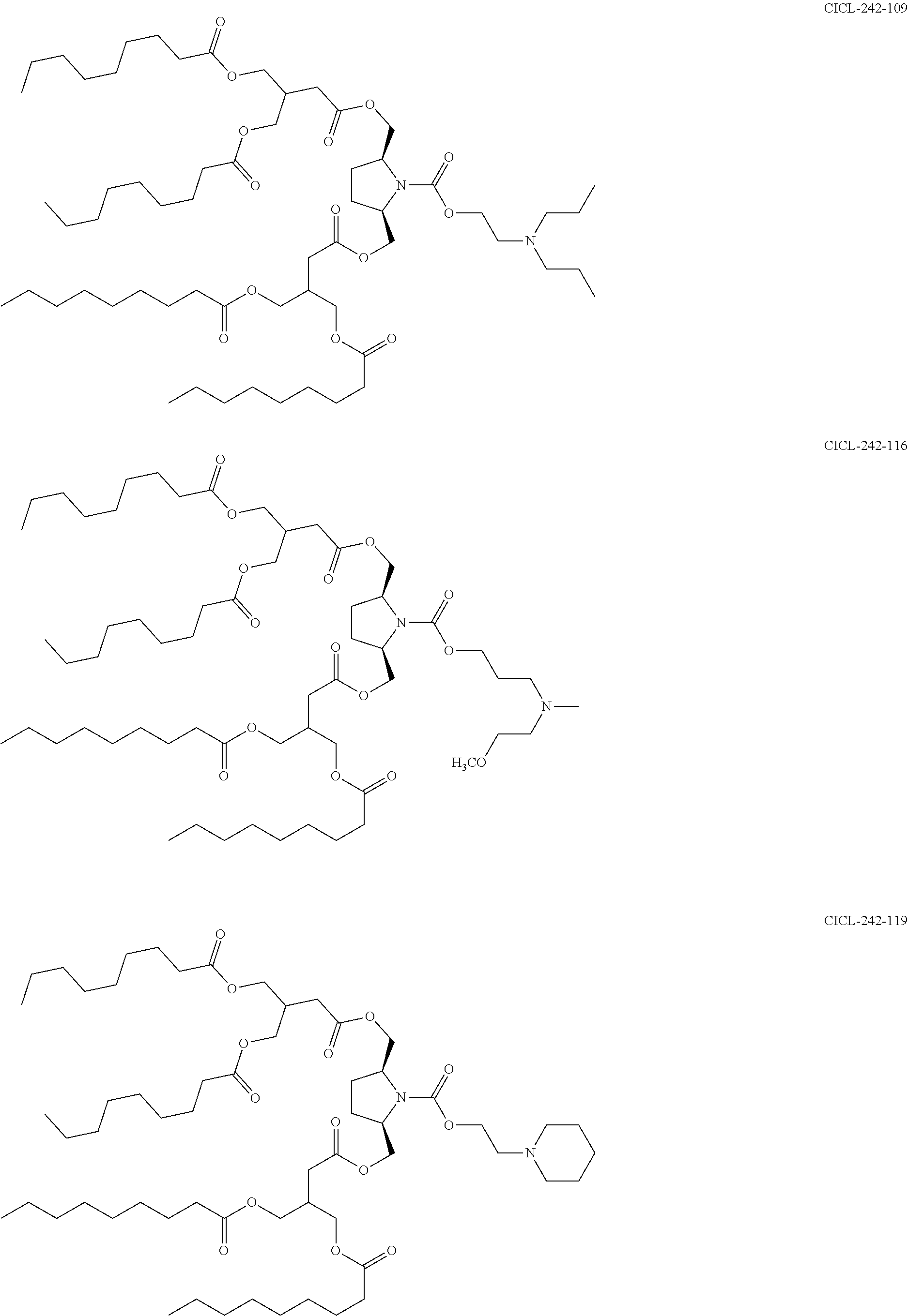

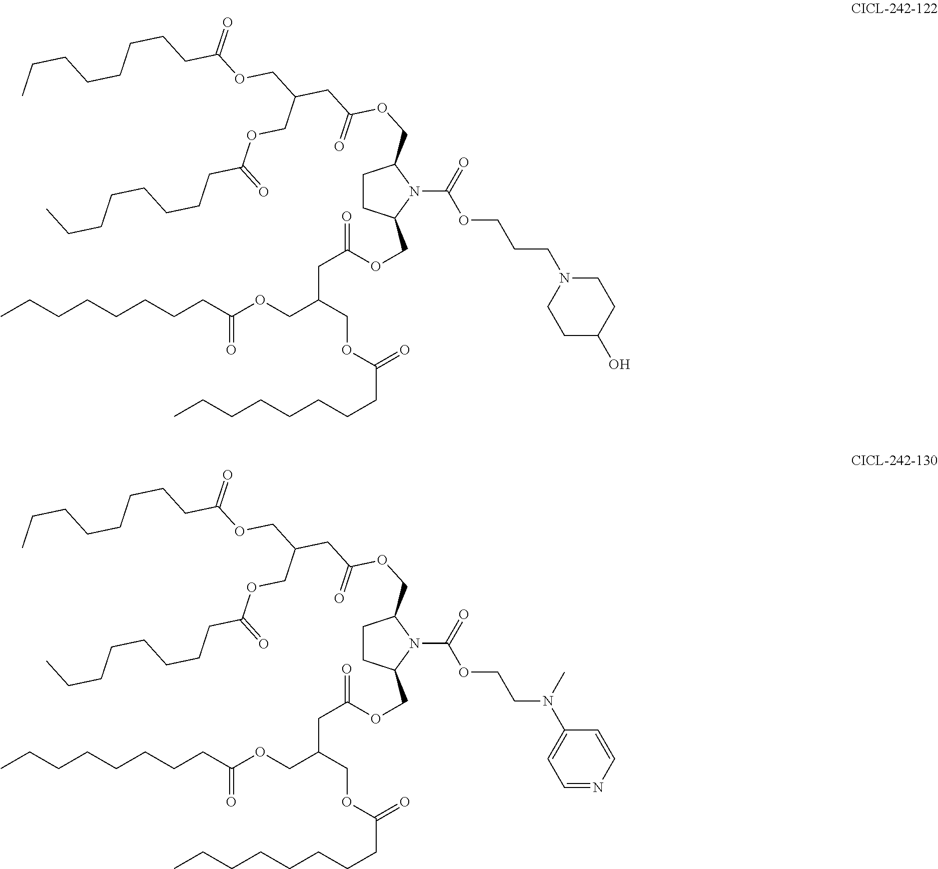 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCCN1CCC(O)CC1 ; CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCN(C)c1ccncc1]

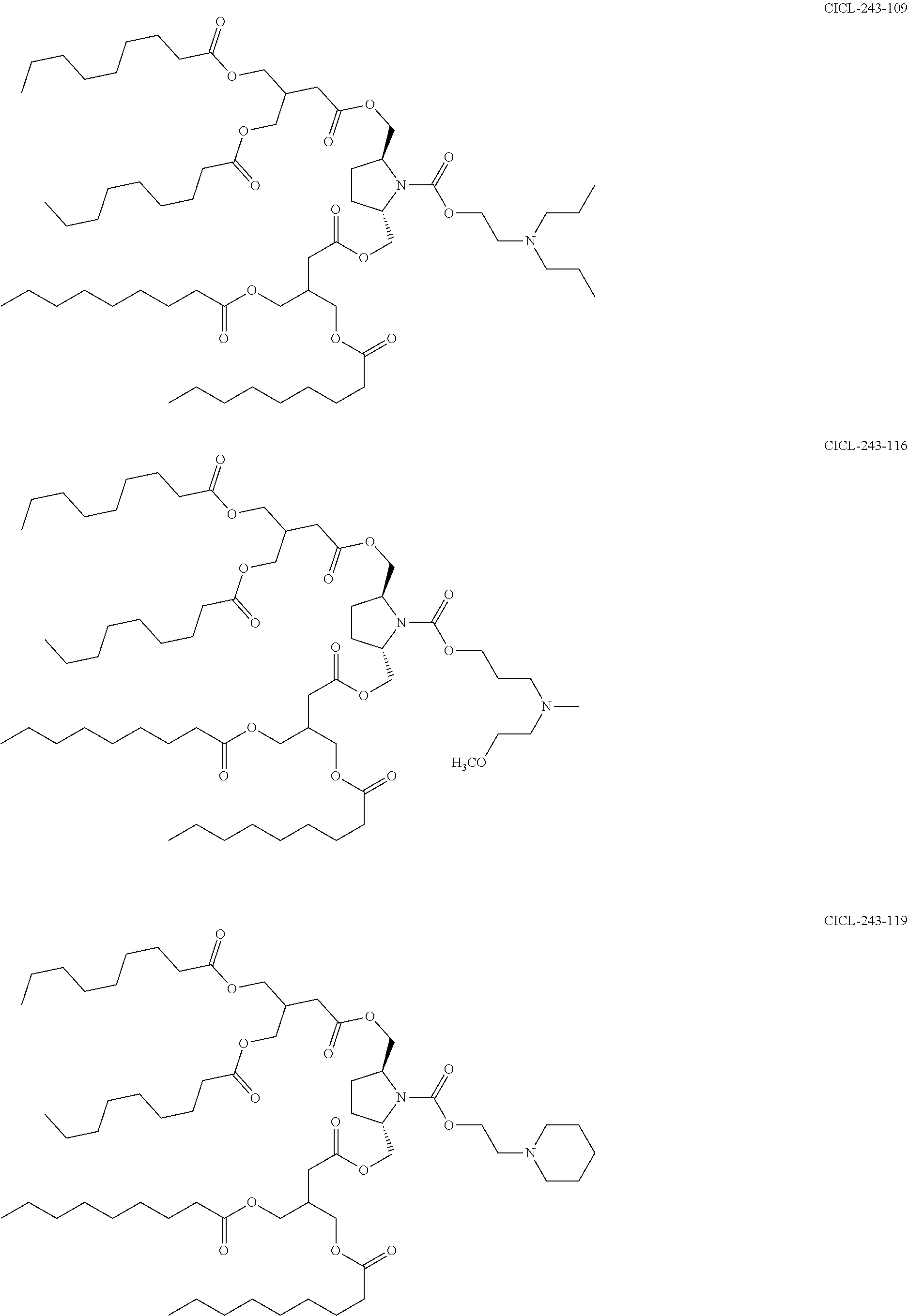

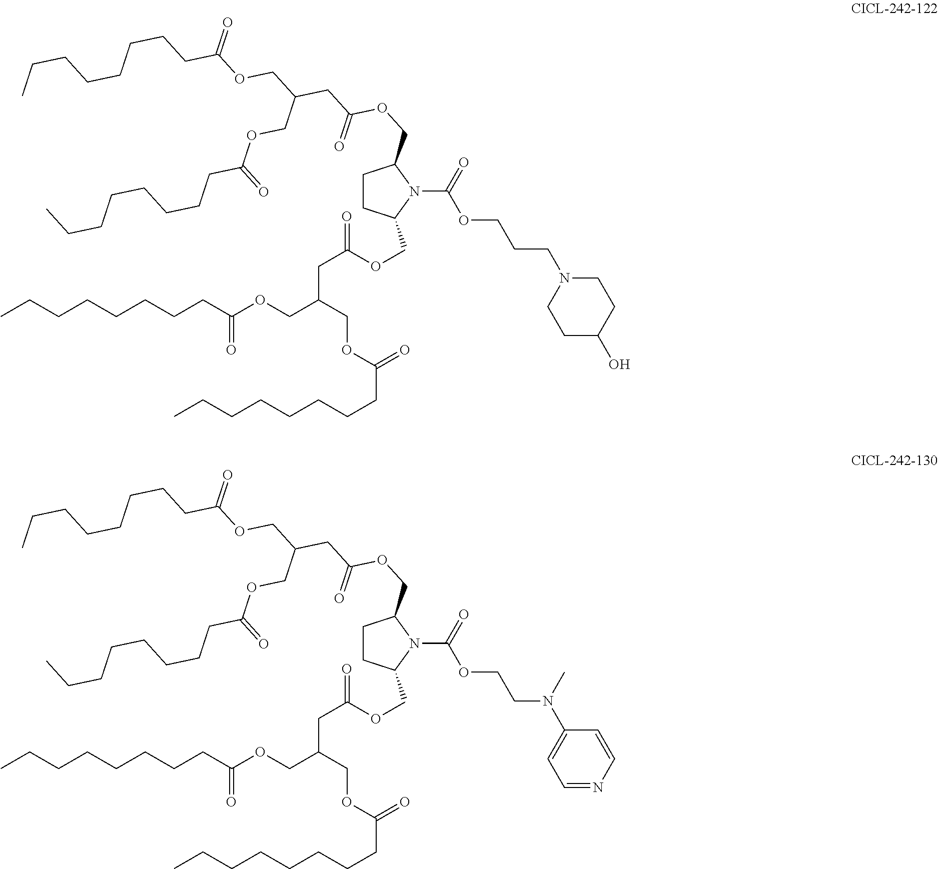 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCCN1CCC(O)CC1 ; CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCN(C)c1ccncc1]

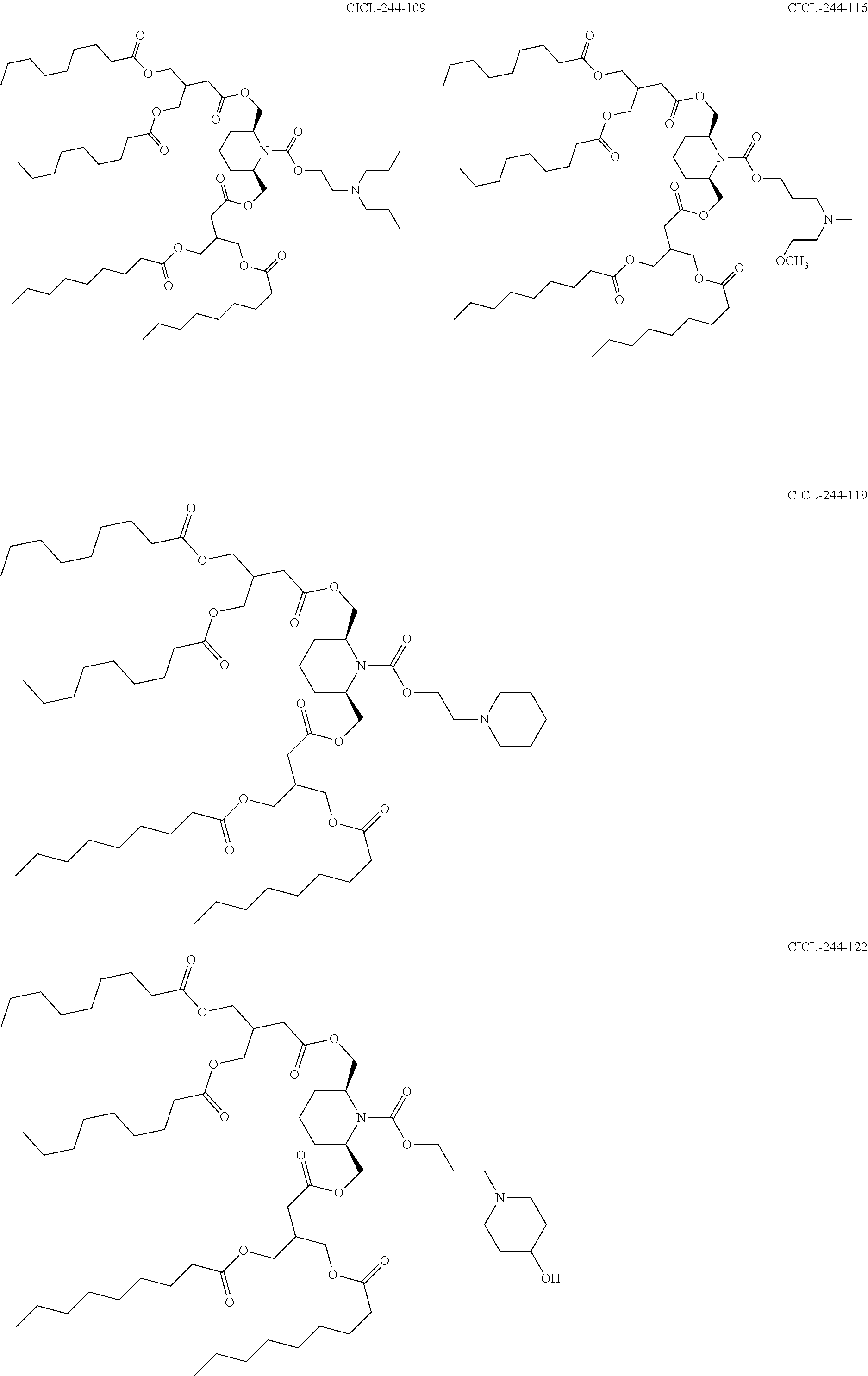

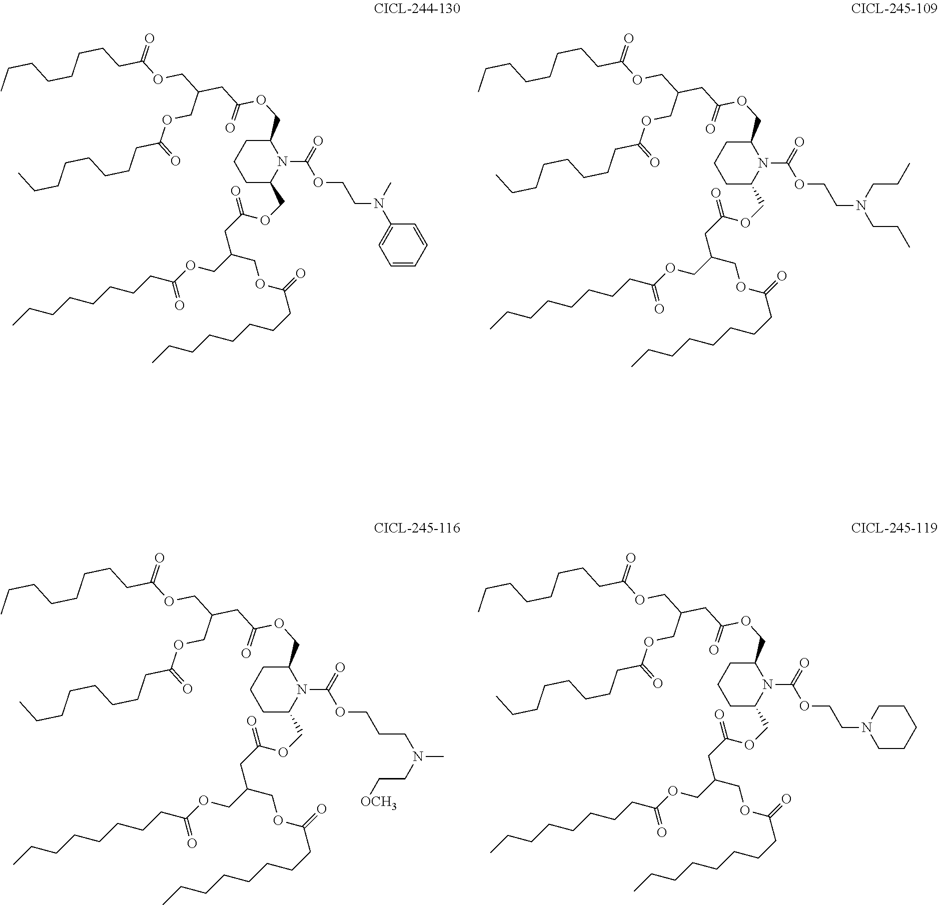 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCN(C)c1ccccc1 ; CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCN(CCC)CCC ; CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCCN(C)CCOC ; CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCN1CCCCC1]

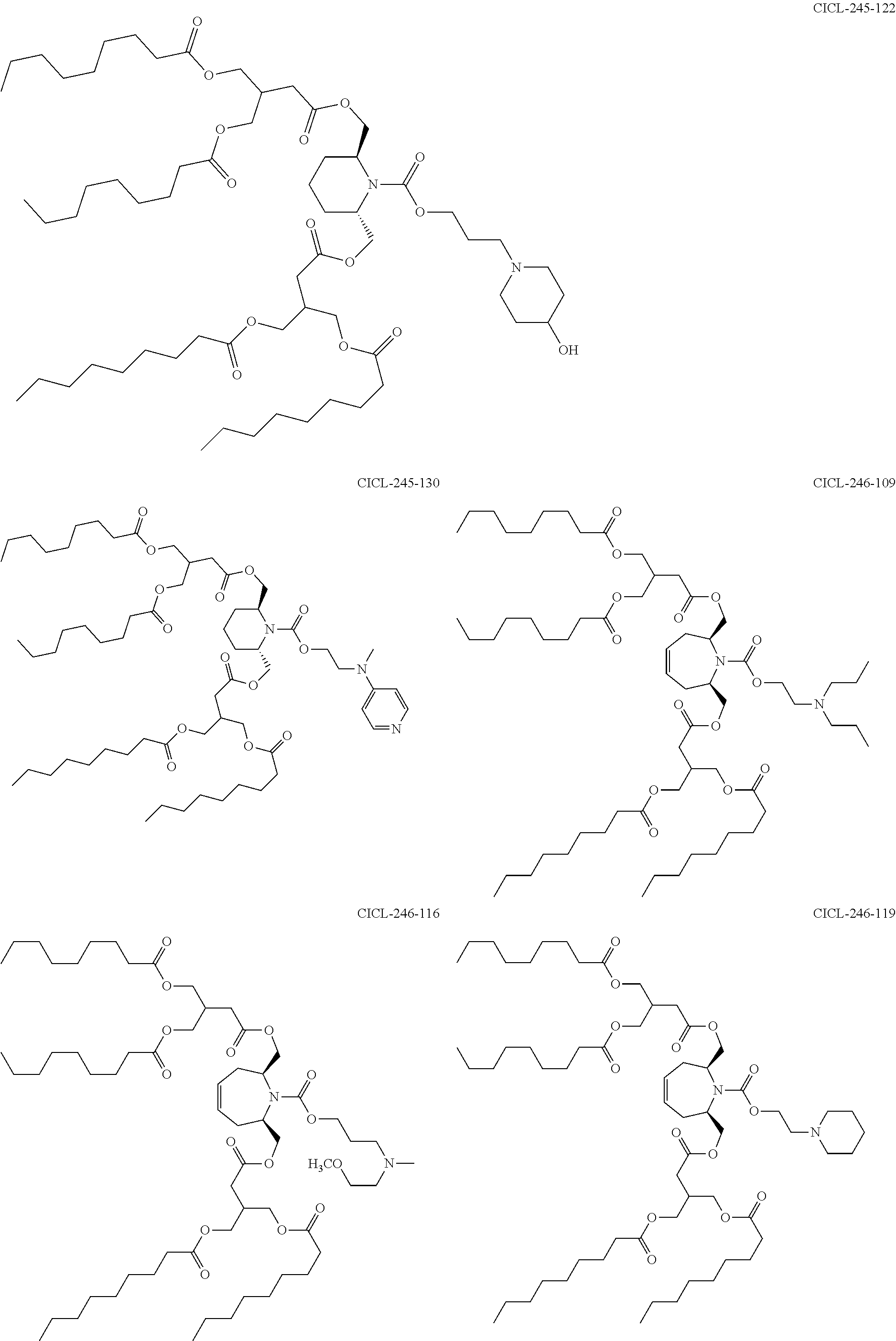

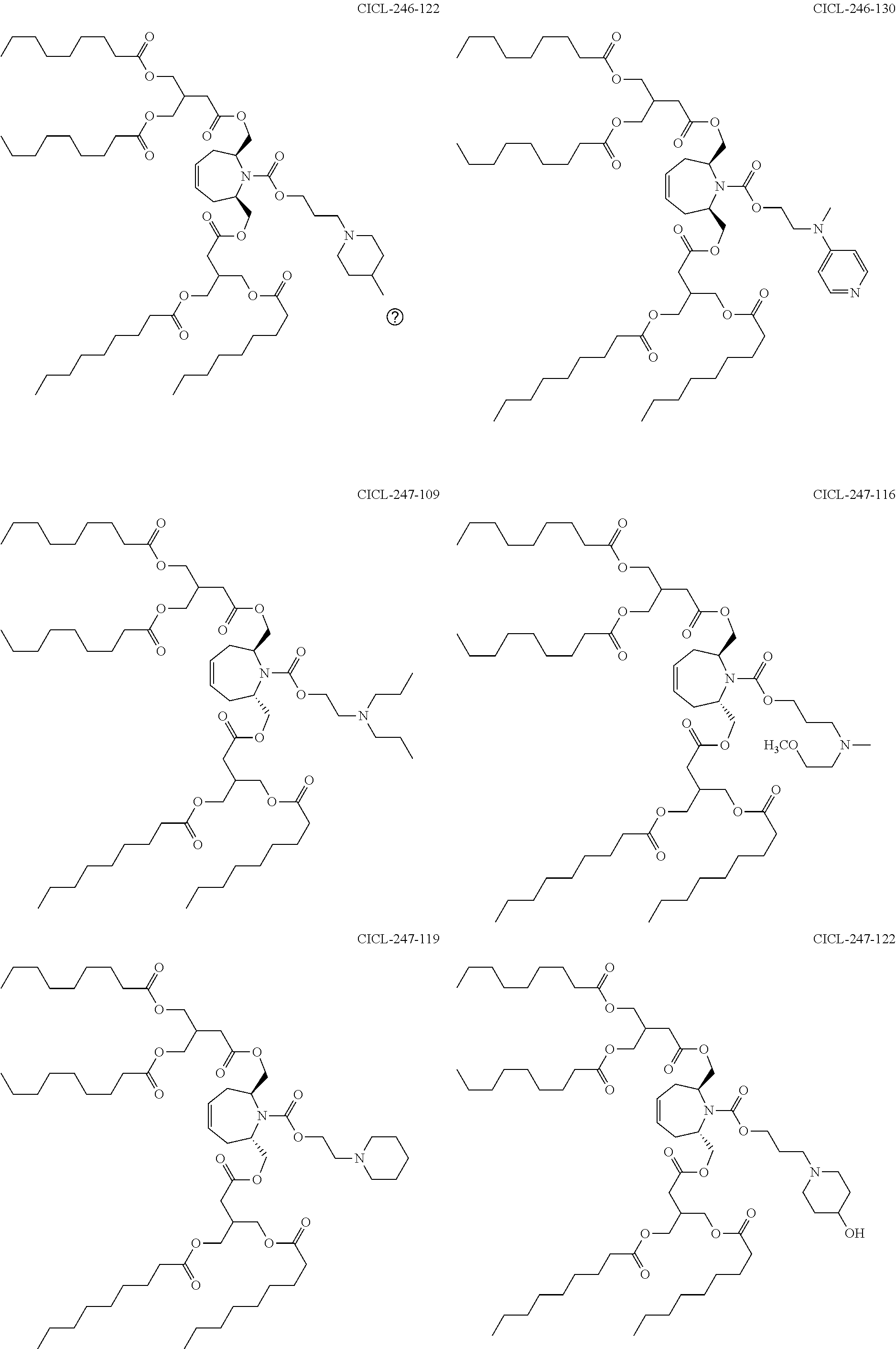

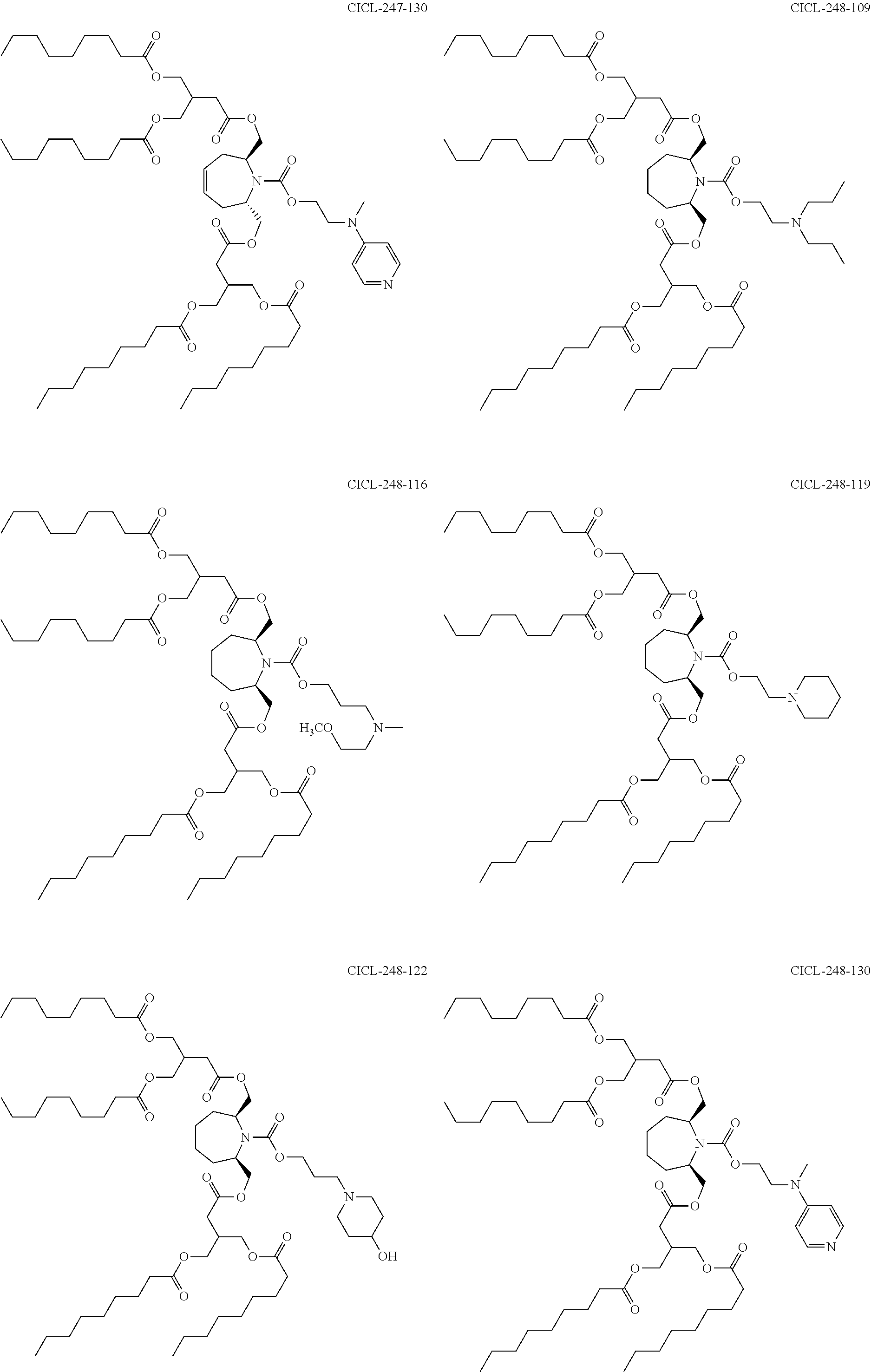

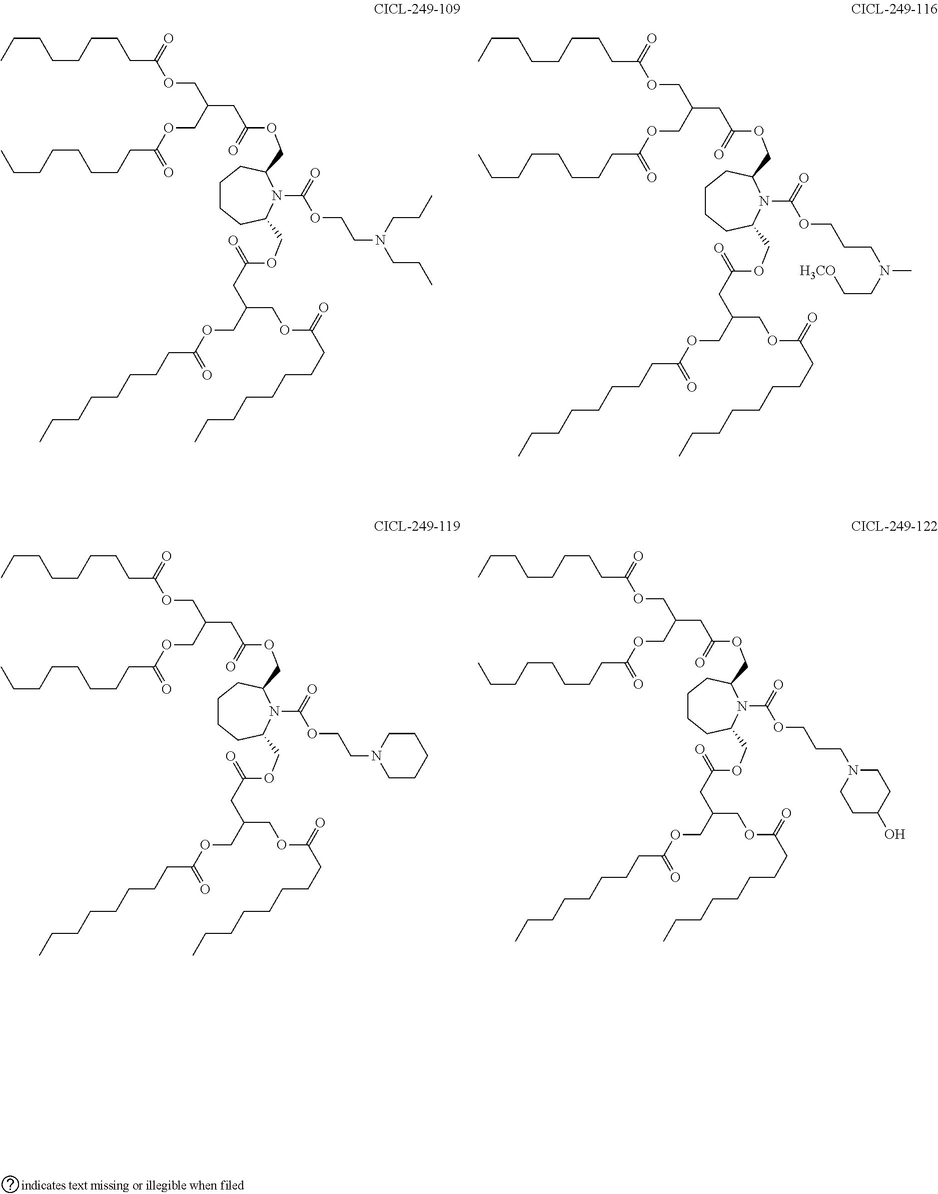 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCN(CCC)CCC ; CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCCN(C)CCOC ; CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCN1CCCCC1 ; CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCCN1CCC(O)CC1]

In other aspects of this disclosure are provided intermediate lipids of the ionizable cationic lipids disclosed herein. In certain aspects, the lipid (e.g., intermediate lipid) of this disclosure have a structure of formula M2-1:

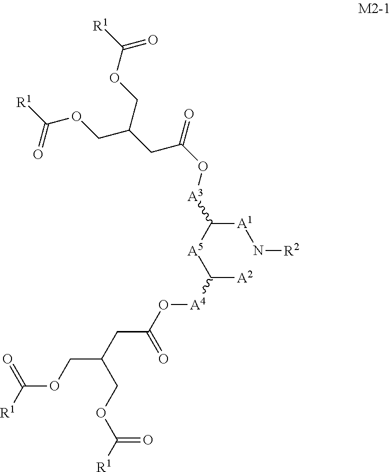

wherein
each R¹ is independently selected from C₇-C₁₁ alkyl or C₇-C₁₁ alkenyl;
R² is H or a protecting group;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
wavy bonds indicate that any relative or absolute stereo-configuration of the corresponding ring atom, or mixtures of stereo-configurations, can be assumed.

In various embodiments as described herein, R² is H.

In various embodiments as described herein, R² is a protecting group (i.e., PG₁). PG₁ can be selected from base labile or acid labile protecting groups as known in the art. For example, in some embodiments, R² is an acid labile protecting group such as t-butoxycarbonyl (BOC) or benzyloxycarbonyl (Cbz). In some other embodiments, R² is a base labile protecting group such as a trimethylsilylethoxycarbonyl moiety. In various embodiments of formula M2-1, R¹, A¹, A², A³, A⁴, and A⁵ are as otherwise described here.

For example, in various embodiments of formula M2-1, A¹ through A⁴ are chosen so that there are only two main chain atoms between the ring nitrogen and each nearest ester oxygen in the nearest tail group.

In certain embodiments of formula M2-1, A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

In certain embodiments of formula M2-1, A¹ is (CH₂)₀, A² is (CH₂)₁, A³ is (CH₂)₁, A⁴ is (CH₂)₀, and A⁵ is (CH₂)₁.

In certain embodiments of formula M2-1, A¹ is (CH₂)₁, A² is (CH₂)₁, A³ is (CH₂)₀, A⁴ is (CH₂)₀, and A⁵ is (CH₂)₀.

In certain embodiments of formula M2-1, A¹ is (CH₂)₁, A² is (CH₂)₁, A³ is (CH₂)₀, A⁴ is (CH₂)₀, and A⁵ is (CH₂)₁.

In certain embodiments of formula M2-1, A¹ is (CH₂)₁, A² is (CH₂)₁, A³ is (CH₂)₀, A⁴ is (CH₂)₀, and A⁵ is (CH₂)₂ or CH═CH.

In certain aspects, the lipid (e.g., intermediate lipid) of this disclosure have a structure of formula M2-2:

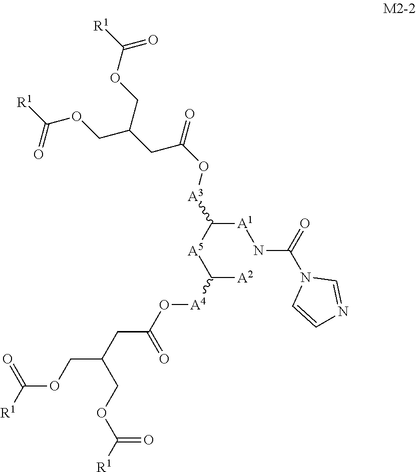

each R¹ is independently selected from C₇-C₁₁ alkyl or C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
wavy bonds indicate that any relative or absolute stereo-configuration of the corresponding ring atom, or mixtures of stereo-configurations, can be assumed.

In various embodiments as described herein, R¹, A¹, A², A³, A⁴, and A⁵ are as otherwise described here.

For example, in various embodiments of formula M2-2, A¹ through A⁴ are chosen so that there are only two main chain atoms between the ring nitrogen and each nearest ester oxygen in the nearest tail group.

In certain embodiments of formula M2-2, A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

In certain embodiments of formula M2-2, A¹ is (CH₂)₀, A² is (CH₂)₁, A³ is (CH₂)₁, A⁴ is (CH₂)₀, and A⁵ is (CH₂)₁.

In certain embodiments of formula M2-2, A¹ is (CH₂)₁, A² is (CH₂)₁, A³ is (CH₂)₀, A⁴ is (CH₂)₀, and A⁵ is (CH₂)₀.

In certain embodiments of formula M2-2, A¹ is (CH₂)₁, A² is (CH₂)₁, A³ is (CH₂)₀, A⁴ is (CH₂)₀, and A⁵ is (CH₂)₁.

In certain embodiments of formula M2-2, A¹ is (CH₂)₁, A² is (CH₂)₁, A³ is (CH₂)₀, A⁴ is (CH₂)₀, and A⁵ is (CH₂)₂ or CH═CH.

To promote biodegradability and minimize the accumulation of ionizable cationic lipids of this disclosure, the fatty acid tails are designed to comprise esters in a position that minimizes steric hindrance of ester cleavage. For example, while a single fatty acid tail will tend to extend away from the ester carbonyl to provide the most energetically favorable position, the presence of two tails leads to the tails extending in opposite directions to provide the most energetically favorable conformation. In certain other embodiments, the fatty acid tails can be in a less energetically favorable position. For example, in certain embodiments, one of the tails extends toward the carbonyl and sterically hinders cleavage of the ester. Accordingly, large branches immediately adjacent to the ester carbonyl were avoided in the design of the cationic lipids disclosed herein. Accordingly, large branches immediately adjacent to the ester carbonyl were avoided. Accordingly, in some embodiments, the ester carbonyls are not in an a position relative to the branch point, for example they are in a β position relative to the branch point. In positioning the ester(s) within the lipid, consideration was also given to potential degradation products to avoid the generation of toxic compounds, such as formaldehyde.

An advantage of relying, at least in part, on ionizable cationic lipids of this disclosure is that it can avoid the toxicity associated with quaternary ammonium cationic lipids. Accordingly, in various embodiments as described herein, a LNP or tLNP does not include a quaternary ammonium (e.g., a quaternary nitrogen group). Some LNPs containing such lipids, which are effectively permanently cationic, have displayed a fatal hyperacute toxicity in laboratory animals. The use of ionizable cationic lipids of this disclosure in an LNP obviates the need for quaternary ammonium cationic lipids and, thereby, can mitigate or avoid potential LNP toxicity. In certain embodiments, use of an LNP or tLNP of this disclosure causes no detectable toxicity to cells or in a subject. In certain embodiments, use of an LNP or tLNP of this disclosure causes no more than mild toxicity to cells or in a subject that is asymptomatic or induces only mild symptoms that do not require intervention. In certain embodiments, use of an LNP or tLNP of this disclosure causes no more than moderate toxicity to cells or in a subject which can impair activities of daily living that requires only minimal, local, or non-invasive interventions.

The relationship between the efficacy and toxicity of a drug is generally expressed in terms of therapeutic window and therapeutic index. Therapeutic window is the dose range from the lowest dose that exhibits a detectable therapeutic effect up to the maximum tolerated dose (MTD); the highest dose that will the desired therapeutic effect without producing unacceptable toxicity. Most typically, therapeutic index is calculated as the ratio of LD50:ED50 when based on animal studies and TD50:ED50 when based on studies in humans (though this calculation could also be derived from animal studies and is sometimes called the protective index), where LD50, TD50, and ED50 are the doses that are lethal, toxic, and effective in 50% of the tested population, respectively. These concepts are applicable whether the toxicity is based on the active agent itself or some other component of the drug product, such as, for example, the LNP or its components. For any inherent level of toxicity of the disclosed lipids or LNPs themselves, an increase in the efficiency of delivering the nucleic acid into the cytoplasm improves the therapeutic window or index, as an effective amount of a biologically active payload (e.g., one or more species of nucleic acid molecule) would be deliverable with a smaller dosage of LNP (and its component lipids).

Toxicities and adverse events are sometimes graded according to a 5-point scale. A grade 1 or mild toxicity is asymptomatic or induces only mild symptoms; can be characterized by clinical or diagnostic observations only; and intervention is not indicated. A grade 2 or moderate toxicity can impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) can be impaired. Grade 4 toxicities are life-threatening and urgent intervention is indicated. Grade 5 toxicity produces an adverse event-related death. Thus, in various embodiments, by use of the disclosed LNP and tLNP a toxicity is confined to grade 2 or less, grade 1 or less, or produces no observation of the toxicity.

### Tolerability

Conventional LNPs deliver primarily to the liver. Liver toxicity has been the major dose limiting parameter observed with LNP-containing pharmaceuticals. For example, ONPATTRO®, comprising the ionizable lipid MC3, has a NOAEL (no observed adverse effect level) of only 0.3 mg/kg for multiple dosing in rats. A benchmark LNP comprising the ionizable cationic lipid ALC-0315, used in the SARS-CoV-2 vaccine COMIRNATY®, caused elevated levels of liver enzymes and acute phase proteins at single doses of 21 mg/kg in the rat. Merely attaching an antibody to the benchmark LNP partially reverses that elevation and the reversal is greater if the antibody directs the LNP to some other tissue (that is, a tLNP). However, use of a highly biodegradable ionizable cationic lipid, CICL-1, the catabolism of which should be similar to those disclosed herein, reduced delivery to the liver and associated liver enzyme and acute phase protein levels to a greater extent for LNP, antibody-conjugated LNP, and tLNP. tLNP comprising CICL-207 disclosed herein were generally well tolerated in rat at single doses of up to at least 8 mg/kg.

### Methods of Making Ionizable Cationic Lipids

Structural symmetries and convergent rather than linear synthesis pathways can be used to simplify the synthesis of the ionizable lipids.

In certain aspects, this disclosure provides a method of synthesizing an ionizable cationic lipid of formula M2 (e.g., without limitation, CICL-207, CICL-215, CICL-216, CICL-217, CICL-218, CICL-219, CICL-220, CICL-221, CICL-222, CICL-223, CICL-224, and CICL-225).

Table 2 provides a summary of substituents in formula M2 for the ring structures of CICL-207, CICL-215, CICL-216, CICL-217, CICL-218, CICL-219, CICL-220, CICL-221, CICL-222, CICL-223, CICL-224, CICL-225, CICL-238, CICL-239, CICL-242, CICL-243, CICL-244, CICL-245, CICL-246, CICL-247, CICL-248, and CICL-249.

TABLE 2
Substituents in formula M2 for the head and ring structures of CICL-207, CICL-215, CICL-216, CICL-217, CICL-218, CICL-219, CICL-220,
CICL-221, CICL-222, CICL-223, CICL-224, CICL-225, CICL-238, CICL-239, CICL-242, CICL-243, CICL-244, CICL-245, CICL-246,
CICL-247, CICL-248, and CICL-249

| Lipid head and ring structure | A¹ | A² | A⁵ | Ring size | A³ | A⁴ | Y |
|---|---|---|---|---|---|---|---|
| 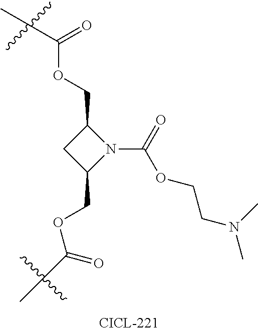 [SMILES: *C(=O)OC[C@@H]1C[C@H](COC(*)=O)N1C(=O)OCCN(C)C] | Absent | Absent | CH₂ | 4 | CH₂ | CH₂ | O—CH₂CH₂—N(CH₃)₂ |
| 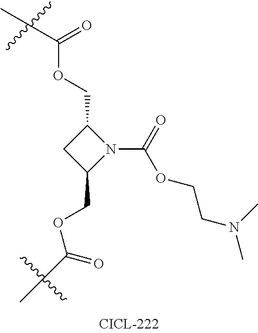 [SMILES: *C(=O)OC[C@H]1C[C@H](COC(*)=O)N1C(=O)OCCN(C)C] | Absent | Absent | CH₂ | 4 | CH₂ | CH₂ | O—CH₂CH₂—N(CH₃)₂ |
| 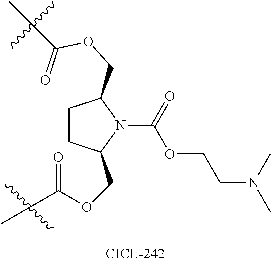 [SMILES: *C(=O)OC[C@H]1CC[C@@H](COC(*)=O)N1C(=O)OCCN(C)C] | Absent | Absent | CH₂CH₂ | 5 | CH₂ | CH₂ | O—CH₂CH₂—N(CH₃)₂ |
| 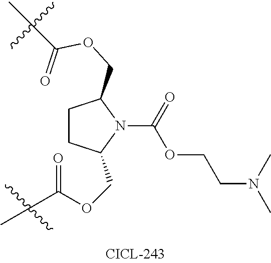 [SMILES: *C(=O)OC[C@@H]1CC[C@@H](COC(*)=O)N1C(=O)OCCN(C)C] | Absent | Absent | CH₂CH₂ | 5 | CH₂ | CH₂ | O—CH₂CH₂—N(CH₃)₂ |
| 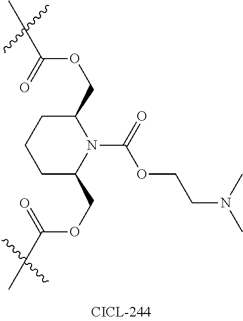 [SMILES: *C(=O)OC[C@H]1CCC[C@@H](COC(*)=O)N1C(=O)OCCN(C)C] | Absent | Absent | CH₂CH₂CH₂ | 6 | CH₂ | CH₂ | O—CH₂CH₂—N(CH₃)₂ |
| 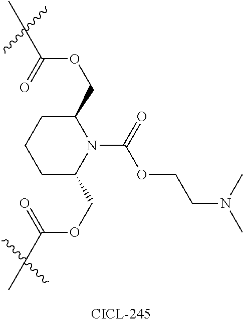 [SMILES: *C(=O)OC[C@@H]1CCC[C@@H](COC(*)=O)N1C(=O)OCCN(C)C] | Absent | Absent | CH₂CH₂CH₂ | 6 | CH₂ | CH₂ | O—CH₂CH₂—N(CH₃)₂ |
| 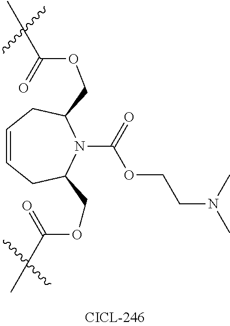 [SMILES: *C(=O)OC[C@H]1CC=CC[C@@H](COC(*)=O)N1C(=O)OCCN(C)C] | Absent | Absent | CH₂CH═CHCH₂ | 7 | CH₂ | CH₂ | O—CH₂CH₂—N(CH₃)₂ |
| 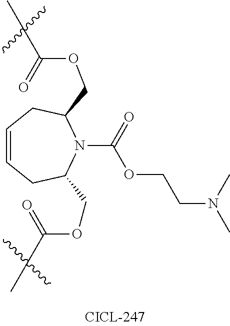 [SMILES: *C(=O)OC[C@@H]1CC=CC[C@@H](COC(*)=O)N1C(=O)OCCN(C)C] | Absent | Absent | CH₂CH═CHCH₂ | 7 | CH₂ | CH₂ | O—CH₂CH₂—N(CH₃)₂ |
| 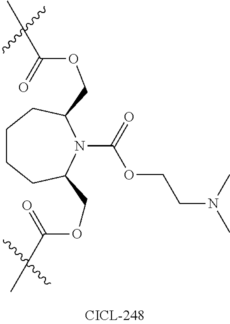 [SMILES: *C(=O)OC[C@H]1CCCC[C@@H](COC(*)=O)N1C(=O)OCCN(C)C] | Absent | Absent | CH₂CH₂CH₂CH₂ | 7 | CH₂ | CH₂ | O—CH₂CH₂—N(CH₃)₂ |
| 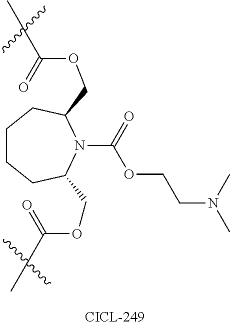 [SMILES: *C(=O)OC[C@@H]1CCCC[C@@H](COC(*)=O)N1C(=O)OCCN(C)C] | Absent | Absent | CH₂CH₂CH₂CH₂ | 7 | CH₂ | CH₂ | O—CH₂CH₂—N(CH₃)₂ |
| 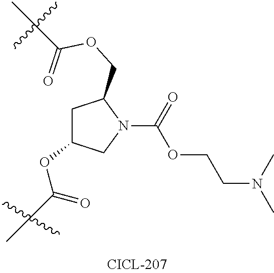 [SMILES: *C(=O)OC[C@@H]1C[C@@H](OC(*)=O)CN1C(=O)OCCN(C)C] | Absent | CH₂ | CH₂ | 5 | CH₂ | Absent | O—CH₂CH₂—N(CH₃)₂ |
| 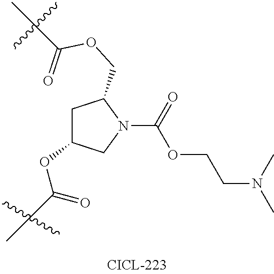 [SMILES: *C(=O)OC[C@H]1C[C@@H](OC(*)=O)CN1C(=O)OCCN(C)C] | Absent | CH₂ | CH₂ | 5 | CH₂ | Absent | O—CH₂CH₂—N(CH₃)₂ |
| 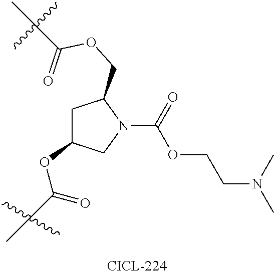 [SMILES: *C(=O)OC[C@@H]1C[C@H](OC(*)=O)CN1C(=O)OCCN(C)C] | Absent | CH₂ | CH₂ | 5 | CH₂ | Absent | O—CH₂CH₂—N(CH₃)₂ |
| 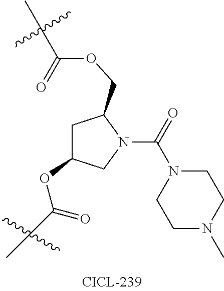 [SMILES: *C(=O)OC[C@@H]1C[C@H](OC(*)=O)CN1C(=O)N1CCN(C)CC1] | Absent | CH₂ | CH₂ | 5 | CH₂ | Absent | 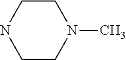 |
| 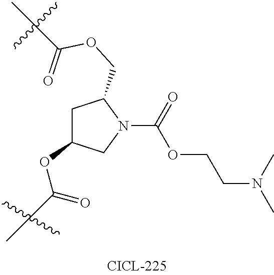 [SMILES: *C(=O)OC[C@H]1C[C@H](OC(*)=O)CN1C(=O)OCCN(C)C] | Absent | CH₂ | CH₂ | 5 | CH₂ | Absent | O—CH₂CH₂—N(CH₃)₂ |
| 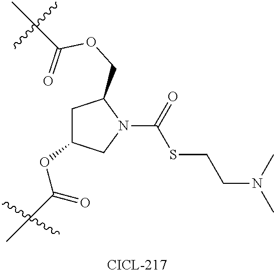 [SMILES: *C(=O)OC[C@@H]1C[C@@H](OC(*)=O)CN1C(=O)SCCN(C)C] | Absent | CH₂ | CH₂ | 5 | CH₂ | Absent | S—CH₂CH₂—N(CH₃)₂ |
| 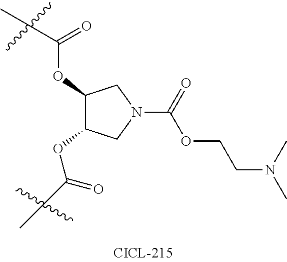 [SMILES: *C(=O)O[C@H]1CN(C(=O)OCCN(C)C)C[C@@H]1OC(*)=O] | CH₂ | CH₂ | Absent | 5 | Absent | Absent | O—CH₂CH₂—N(CH₃)₂ |
| 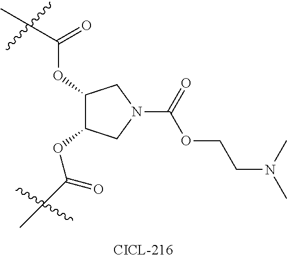 [SMILES: *C(=O)O[C@H]1CN(C(=O)OCCN(C)C)C[C@H]1OC(*)=O] | CH₂ | CH₂ | Absent | 5 | Absent | Absent | O—CH₂CH₂—N(CH₃)₂ |
| 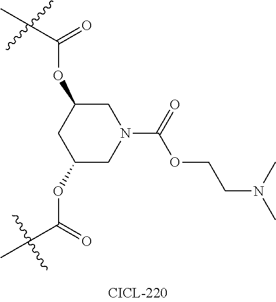 [SMILES: *C(=O)O[C@@H]1C[C@@H](OC(*)=O)CN(C(=O)OCCN(C)C)C1] | CH₂ | CH₂ | CH₂ | 6 | Absent | Absent | O—CH₂CH₂—N(CH₃)₂ |
| 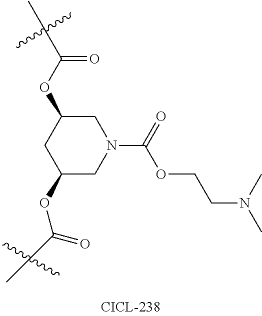 [SMILES: *C(=O)O[C@@H]1C[C@H](OC(*)=O)CN(C(=O)OCCN(C)C)C1] | CH₂ | CH₂ | CH₂ | 6 | Absent | Absent | O—CH₂CH₂—N(CH₃)₂ |
| 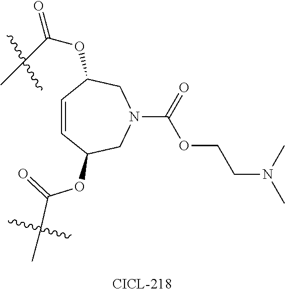 [SMILES: *C(=O)O[C@H]1C=C[C@H](OC(*)=O)CN(C(=O)OCCN(C)C)C1] | CH₂ | CH₂ | CH═CH | 7 | Absent | Absent | O—CH₂CH₂—N(CH₃)₂ |
| 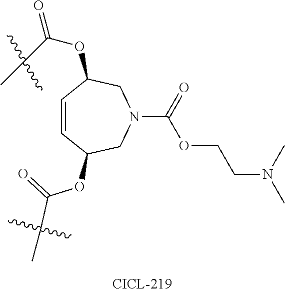 [SMILES: *C(=O)O[C@@H]1C=C[C@H](OC(*)=O)CN(C(=O)OCCN(C)C)C1] | CH₂ | CH₂ | CH═CH | 7 | Absent | Absent | O—CH₂CH₂—N(CH₃)₂ |

In certain embodiments, this disclosure provides a method of synthesizing an ionizable cationic lipid of formula M2 comprising the synthesis step as shown in Scheme M2, wherein A⁻ is an anion of an acid AH, and the rest of the substituents are defined the same as in formula M2. In certain embodiments, the method further comprises the synthesis step shown in Scheme 4-A. A particular stereoisomer of the ionizable cationic lipid of formula M2 can be prepared according to the synthesis method disclosed herein with starting materials and/or intermediates having the same stereochemistry.

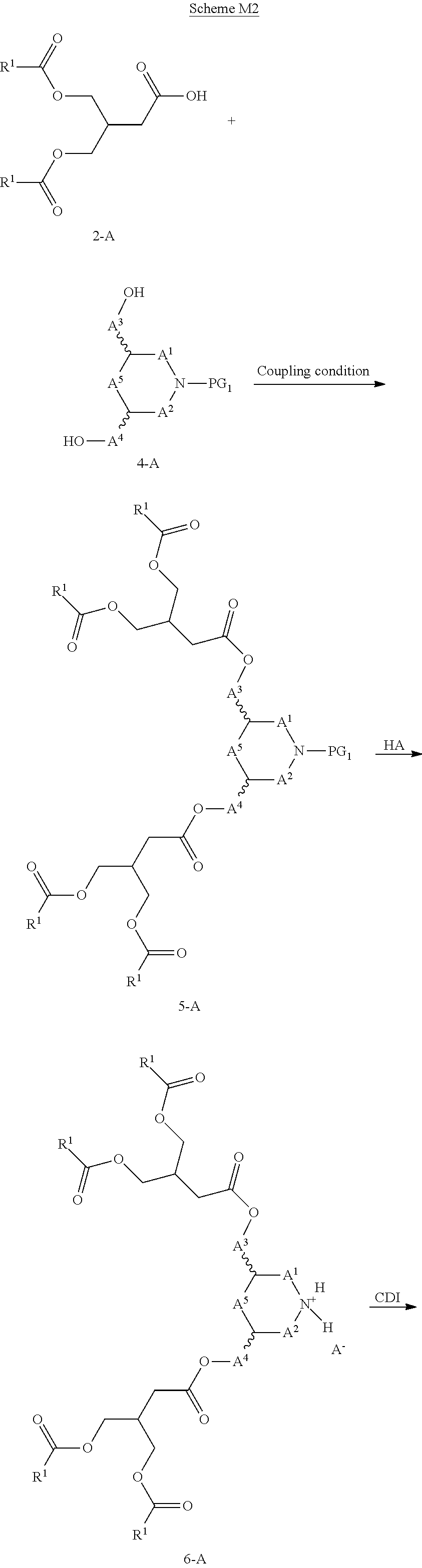

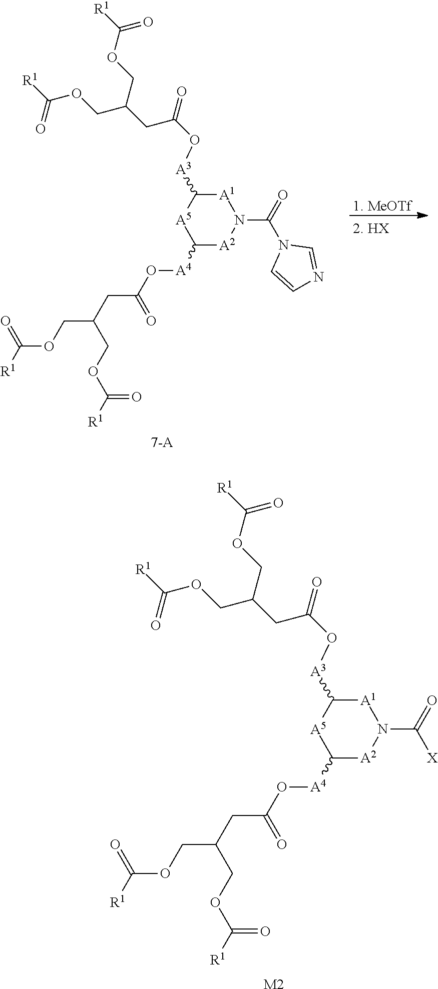

The synthesis step shown in Scheme M2 comprises reacting a diester-amine 6-A with 1,1′-carbonyldiimidazole (CDI) to provide an imidazolecarboxyamide 7-A; and after activation of the imidazolecarboxyamide 7-A it is coupled with a desired alcohol/thiol/amine (HX) to provide a corresponding carbamate/thiocarbamate/urea having the structure of formula M2, all substituents are defined the same as those in formula M2.

In certain embodiments, the imidazolecarboxyamide 7-A synthesis is carried out in an organic solvent (e.g., without limitation, CH₂Cl₂) in the presence of a basic catalyst (e.g., without limitation, trimethylamine). In certain embodiments, the carbamate/thiocarbamate/urea synthesis can comprise first reacting the imidazolecarboxyamide 7-A with trifluoromethanesulfonate (MeOTf), then reacting with the desired alcohol/thiol/amine (HX) in the presence of a base (e.g., trimethylamine). The reaction can be carried out in an organic solvent (e.g., acetonitrile). In certain embodiments as described herein, HX is an alcohol, wherein X is as described herein. In certain embodiments as described herein, HX is a thiol, wherein X is as described herein. In certain embodiments as described herein, HX is an amine, wherein X is as described herein. Accordingly, in some embodiments, HX is an alcohol/thiol/amine that provides that desired X group to the lipids of formula M2. For example, in various embodiments as described herein, HX is selected from

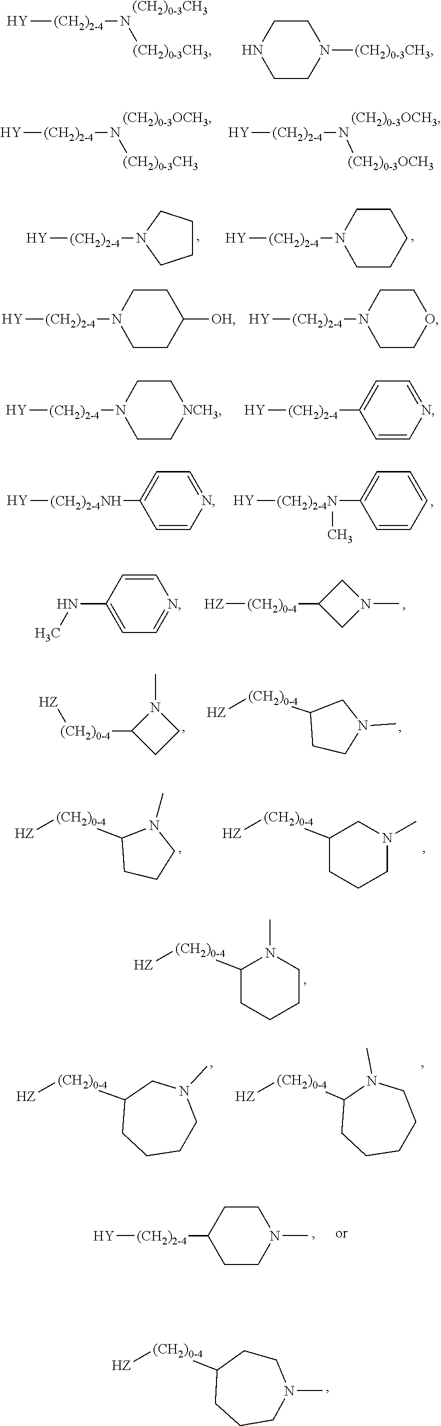

wherein Y is O, S, NH, of NCH₃ and Z is O, NH, NCH₃. The various HX compounds disclosed herein are commercially available, are known in the scientific literature, or can be made using procedures familiar to the person of ordinary skill in the art, provided from commercial sources, or the general procedures described in the Examples below.

For example, in various embodiments, HX is selected from any one of

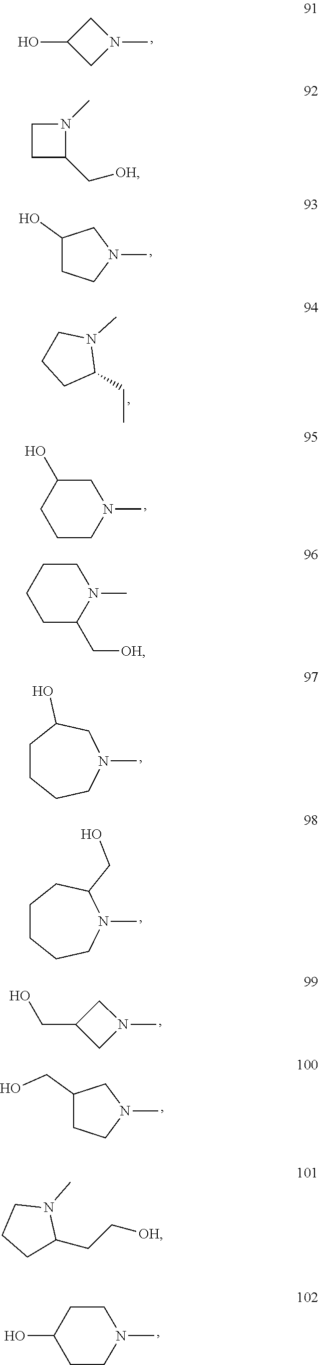

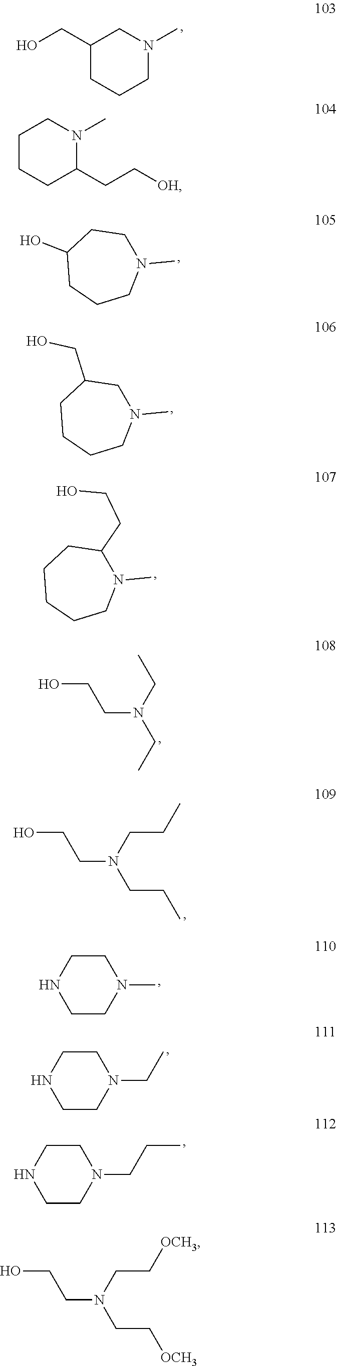

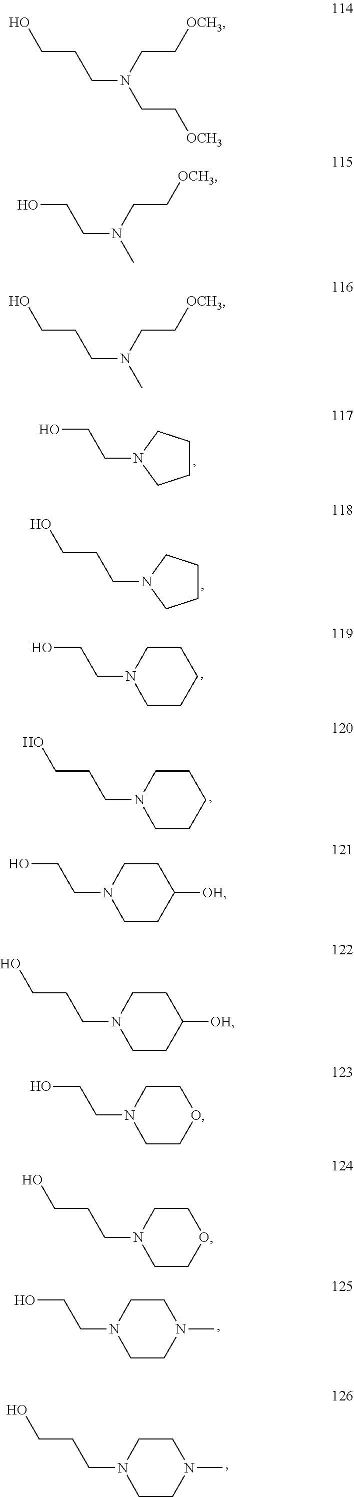

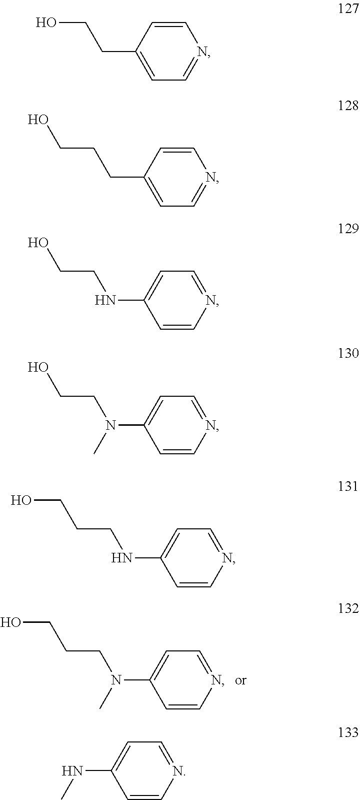

In certain embodiments, the method can further comprise synthesis of the diester-amine 6-A comprising deprotecting a protected diester-amine 5-A to provide the unprotected diester-amine 6-A. The deprotection can be carried out under acidic or basic conditions, depending on the protecting group (PG₁). For example, the deprotection step can be carried out under an acidic condition (e.g., in the presence of an acid AH, such as trifluoroacetic acid, TFA, when the protecting groups is an acid labile protecting group, e.g., t-butoxycarbonyl (BOC)) in an organic solution (e.g., CH₂Cl₂). Other PG₁ and deprotection steps can be used as known to the person of ordinary skill in the art. For example, PG₁ can be a benzyloxycarbonyl (Cbz) that can be removed in the presence of hydrogen and a Pd/C catalyst. A base labile PG₁, such as trimethylsilylethoxycarbonyl moiety, can also be used and be removed with nBuNH₄ or HF-pyridine.

In certain embodiments, the method further comprises synthesis of the protected diester-amine 5-A comprising coupling an unprotected diester-acid 2-A with a desired diol-amine protected by a first protecting group (PG₁) (diol-protected amine 4-A) to form protected diester-amine 5-A. In certain embodiments, the coupling reaction is carried out in an organic solvent (e.g., acetonitrile) in the presence of a nucleophilic catalyst (e.g., DMAP) and an acidic catalyst (e.g., 1-(3-Dimethylaminopropyl)-3-ethylcarbodiimide hydrochloride (EDC-HCl)). In certain embodiments, PG₁ can be —C(═O)—O—C(CH₃)₃.

In certain embodiments, the method further comprises synthesis of the diol-protected amine 4-A as shown in Scheme 4-A, comprising reacting a benzyl diol-amine 3-A with a dicarbonate (e.g., (PG₁)₂O such as di-tert-butyl dicarbonate (BOC₂O)) in the presence of a catalyst (e.g., Pd(OH)₂) and hydrogen to provide the diol-protected amine 4-A.

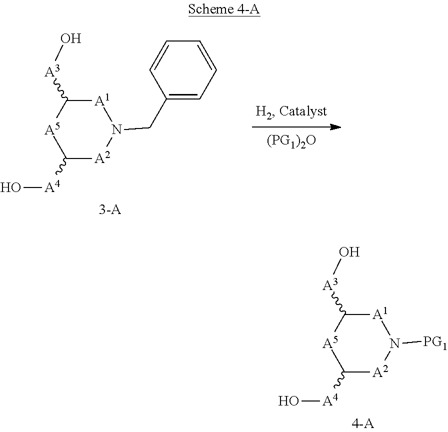

The syntheses are described using specific solvents, but in all cases alternative solvents are known to the person of skill in the art. THF can be substituted, for example, without limitation, by DMF, diethyl ether, methyl t-butyl ether, dioxane, or 2-methyl THF. Ethyl acetate can be substituted by, for example, without limitation, isopropyl acetate, THF, 2-methyl THF, dioxane, or methyl t-butyl ether. Dichloromethane can be substituted by, for example, without limitation, ethyl acetate, isopropyl acetate, THF, methyl t-butyl ether, 2-methly THF, dioxane, or heptane. Methanol can be substituted by, for example, without limitation, ethanol, or aqueous THF. Acetonitrile can be substituted by, for example, THF, 2-methyl THF, dichloromethane, ethyl acetate, isopropyl acetate, methyl t-butyl ether, or toluene.

Many general references providing commonly known chemical synthetic schemes and conditions useful for synthesizing materials/intermediates used in the synthesis of the disclosed compounds are available (see, e.g., Smith, March's Advanced Organic Chemistry: Reactions, Mechanisms, and Structure, Eight Edition, Wiley-Interscience, 2019; or Fumiss, Hannaford, Smith, Tatchelll, Vogel's Textbook of Practical Organic Chemistry, Including Qualitative Organic Analysis, Fifth Edition, New York: Longman, 1989).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically, the disclosed compounds are purified via silica gel and/or alumina chromatography. See, e.g., Still, Kahn, Mitra, J. Org. Chem. 1978, 43, 2923-292, Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it can be necessary and/or desirable to protect sensitive or reactive groups on any of the molecules concerned. This can be achieved by means of conventional protecting groups as described in standard works, such as J. F. W. McOmie, “Protective Groups in Organic Chemistry,” Plenum Press, London and New York 1973, in T P. G. M. Wuts, “Greene's Protective Groups in Organic Synthesis,” Firth edition, Wiley, New York 2014, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups can be removed at a convenient subsequent stage using methods known from the art.

The intermediate compounds of the ionizable cationic lipids disclosed herein can be made using procedures familiar to the person of ordinary skill in the art and as described herein. For example, compounds of structural formula M2 can be prepared according to Scheme M2, Scheme 4A, general procedures (see the Examples below), and/or analogous synthetic procedures. One of skill in the art can adapt the reaction sequences of Schemes M2 and 4A, general procedures, and Examples described to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

Lipid Nanoparticles (LNPs) and Targeted LNPs (tLNPs)

In certain aspects, this disclosure provides an LNP comprising an ionizable cationic lipid of formula M2. In some embodiments, an LNP comprises an ionizable cationic lipid of formula M2 and a phospholipid, a sterol, a co-lipid, a PEGylated lipid, or a combination thereof. In certain embodiments, the PEG-lipids are not functionalized PEG-lipids. In other embodiments, the PEG-lipids are functionalized PEG-Lipids. In certain embodiments, the LNP comprises at least one PEG-lipid that is functionalized and at least one PEG-lipid that is not functionalized.

In further aspects, this disclosure provides a targeted lipid nanoparticle (tLNP) comprising an ionizable cationic lipid of formula M2. In some embodiments, the aforementioned tLNP can further comprise one or more of a phospholipid, a sterol, a co-lipid, and a PEG-lipid, or a combination thereof, and a functionalized PEG-lipid. As used herein, “functionalized PEG-lipid” refers to a PEG-lipid in which the PEG moiety has been derivatized with a chemically reactive group that can be used for conjugating a targeting moiety to the PEG-lipid. The functionalized PEG-lipid can be reacted with a binding moiety after the LNP is formed, so that the binding moiety is conjugated to the PEG portion of the lipid. The conjugated binding moiety can thus serve as a targeting moiety for the tLNP.

In various embodiments, a binding moiety of a LNP (or tLNP) comprises an antigen binding domain of an antibody, an antigen, a ligand-binding domain of a receptor, or a receptor ligand. In some embodiments, the binding moiety comprising an antigen binding domain of an antibody comprises a complete antibody, an F(ab)₂, an Fab, a minibody, a single-chain Fv (scFv), a diabody, a VH domain, or a nanobody, such as a VHH or single domain antibody. In some embodiments, the receptor ligand is a carbohydrate, for example, a carbohydrate comprising terminal galactose or N-acetylgalactosamine units, which are bound by the asialoglycoprotein receptor. These binding moieties constitute means for LNP targeting. Some embodiments specifically include one or more of these binding moieties. Other embodiments specifically exclude one or more of these binding moieties.

LNP and tLNP Compositions

The LNP composition contributes to the formation of stable LNPs and tLNPs, efficient encapsulation of a payload, protection of a payload from degradation until it is delivered into a cell, and promotion of endosomal escape of a payload into the cytoplasm. These functions are primarily independent of the specificity of the binding moiety (or moieties) serving to direct or bias a tLNP to a particular cell type(s). Additional LNP and tLNP compositions are generally disclosed in PCT/US2024/032141, filed 31 May 2024 and entitled Lipid Nanoparticle Formulations and Composidons, which is incorporated by reference for all that it teaches about the design, formation, characterization, properties, and use of LNPs and tLNPs.

The LNPs and/or tLNPs can include the various components in amounts sufficient to provide a nanoparticle with a desired shape, fluidity, and bio-acceptability as described herein. With respect to LNPs or tLNPs of this disclosure, in some embodiments, the LNP (or tLNP) comprises at least one ionizable cationic lipid (e.g., as described herein) in an amount in the range of from about 35 to about 65 mol % or any integer bound sub-range thereof, e.g., in an amount of from about 40 to about 65 mol %, about 40 to about 60 mol % or about 40 mol % to about 62 mol %. In some embodiments, the LNP or tLNP comprises about 58 mol %, about 60 mol %, or 62 mol % ionizable cationic lipid. In some embodiments, the LNP (or tLNP) comprises a phospholipid in an amount in the range of from about 7 to about 30 mol % or any integer bound sub-range thereof, e.g., in an amount of from about 13 to about 30 mol %. In some embodiments, the LNP or tLNP comprises about 10 mol % phospholipid. In some embodiments, the LNP (or tLNP) comprises a sterol in an amount in the range of from about 20 to about 50 mol % or any integer bound sub-range thereof, e.g., in an amount in the range of from about 20 to about 45 mol %, or about 30 to about 50 mol %, or about 30 to about 45 mol %. In some embodiments, the LNP or tLNP comprises about 30.5, 26.5, or 23.5 mol % sterol. In some embodiments, the LNP (or tLNP) comprises at least one co-lipid in an amount in the range of from about 1 to about 30 mol %. In some embodiments, an LNP or tLNP comprises total PEG-lipid in an amount in the range of from about 1 mol % to about 5 mol % or any integer×10⁻¹ bound sub-range thereof, e.g., in an amount in the range of from about 1 mol % to about 2 mol % total PEG-lipid. In some embodiments, the LNP (or tLNP) comprises at least one unfunctionalized PEG-lipid in an amount of from 0 to about 5 mol % or any integer×10⁻¹ bound sub-range thereof, e.g., in the range of amount 0 to about 3 mol %, or about 0.1 to about 5 mol %, or about 0.5 to about 5 mol %, or about 0.5 to about 3 mol %. In some embodiments, the LNP or tLNP comprises about 1.4 mol % unfunctionalized PEG-lipid. In some embodiments, the LNP or tLNP comprises at least one functionalized PEG-lipid in an amount in the range of from about 0.1 to about 5 mol % or any integer×10⁻¹ bound sub-range thereof, e.g., in the range of from about 0.1 to 0.3 mol %. In certain embodiments, an LNP or tLNP comprises about 0.1 mol %, about 0.2 mol %, or about 0.3 mol % functionalized PEG-lipid. In some embodiments, the LNP or tLNP comprises about 0.1 mol % functionalized PEG-lipid. In some embodiments, the functionalized PEG-lipid is conjugated to a binding moiety. In certain instances, a tLNP is an LNP that further comprises an antibody (for example, a whole IgG) as the binding moiety which is present at an antibody:mRNA ratio (w/w) of about 0.3 to about 1.0.

In certain aspects, this disclosure provides an LNP or tLNP, wherein the LNP or tLNP comprises about 35 mol % to about 65 mol % of an ionizable cationic lipid, about 0.5 mol % to about 3 mol % of a PEG-lipid (including non-functionalized PEG-lipid and optionally a functionalized PEG-lipid), about 7 mol % to about 13 mol % of a phospholipid, and about 30 mol % to about 50 mol % of a sterol. In some embodiments, an LNP or tLNP comprises a payload with a net negative charge for example, a peptide, a polypeptide, a protein, a small molecule, or a nucleic acid molecule, and combinations thereof. A payload is generally encompassed by or in the interior of an LNP or tLNP. As disclosed herein dosages always refer to the amount of payload being provided. In some embodiments, a payload comprises one or more species of nucleic acid molecule. For tLNP encapsulating mRNA dosages are typically in the range of 0.05 to 5 mg/kg without regard for recipient species. In some embodiments, the dosage is in the range of 0.1 to 1 mg/kg.

With respect to LNPs or tLNPs of this disclosure, in some embodiments, the ratio of total lipid to nucleic acid is about 10:1 to about 50:1 on a weight basis. In some embodiments, the ratio of total lipid to nucleic acid is about 10:1, about 20:1, about 30:1, or about 40:1 to about 50:1, or 10:1 to 20:1, 30:1, 40:1 or 50:1, or any range bound by a pair of these ratios. The ratio of lipid to nucleic acid can also be reported as an N/P ratio, the ratio of positively chargeable lipid amine (N=nitrogen) groups to negatively-charged nucleic acid molecule phosphate (P) groups. In some embodiments, the N/P ratio is from about 3 to about 9, about 3 to about 7, about 3 to about 6, about 4 to about 6, about 5 to about 6, or about 6. In some embodiments, the N/P ratio is from 3 to 9, 3 to 7, 3 to 6, 4 to 6, 5 to 6, or 6. In certain embodiments as described herein, the LNP (or tLNP) comprises a binding moiety, wherein the binding moiety comprises an antigen binding domain of an antibody and wherein the antibody is a whole antibody and the ratio of a lipid to nucleic acid is in the range of from about 0.3 to about 1.0 w/w.

Due to physiologic and manufacturing constraints LNP or tLNP, particles with a hydrodynamic diameter of about 50 to about 150 nm are desirable for in vivo use. Accordingly, in some embodiments, the LNP or tLNP has a hydrodynamic diameter of 50 to 150 nm and in some embodiments the hydrodynamic diameter is ≤120, ≤110, ≤100, or ≤90 nm. Uniformity of particle size is also desirable with a polydispersity index (PDI) of ≤0.2 (on a scale of 0 to 1) being acceptable. Both hydrodynamic diameter and polydispersity index are determined by dynamic light scattering (DLS). Particle diameter as assessed from cryo-transmission electron microscopy (Cryo-TEM) can be smaller than the DLS-determined value.

### Phospholipids

As described above, in various embodiments, the LNPs and tLNPs include a phospholipid. As would be understood by the person or ordinary skill in the art, phospholipids are amphiphilic molecules. Due to the amphiphilic nature of phospholipids, these molecules are known to form bilayers and by including them in the LNPs and tLNPs, as described herein, they can provide membrane formation, stability, and rigidity. As used herein, phospholipids include a hydrophilic head group including a functionalized phosphate group, and two hydrophobic tail groups derived from fatty acids. For example, in various embodiments as described herein, the phospholipids include a phosphate group functionalized with ethanolamine, choline, glycerol, serine, or inositol. As described above, the phospholipid includes two hydrophobic tail groups derived from fatty acids. These hydrophobic tail groups can be derived from unsaturated or saturated fatty acids. For example, the hydrophobic tail groups can be derived from a C₁₂-C₂₀ fatty acid. With respect to LNPs or tLNPs of this disclosure, in various embodiments, a phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof. In various embodiments, the phospholipid is dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC). In some embodiments, the phospholipid is distearoylphosphatidylcholine (DSPC). Phospholipids can contribute to formation of a membrane, whether monolayer, bilayer, or multi-layer, surrounding the core of the LNP or tLNP. Additionally, phospholipids such as DSPC, DMPC, DPPC, DAPC impart stability and rigidity to membrane structure. Phospholipids, such as DOPE, impart fusogenicity. Further phospholipids, such as DMPG, which attain negative charge at physiologic pH, facilitates charge modulation. Thus, phospholipids constitute means for facilitating membrane formation, means for imparting membrane stability and rigidity, means for imparting fusogenicity, and means for charge modulation.

In some embodiments, an LNP or tLNP has about 7 mol % to about 13 mol % phospholipid, about 7 mol % to about 10 mol % phospholipid, or about 10 mol % to about 13 mol % phospholipid. In certain embodiments, an LNP has about 7 mol %, about 10 mol %, or about 13 mol % phospholipid. In certain instances, the phospholipid is DSPC. In certain instances, the phospholipid is DAPC.

### Sterols

The disclosed LNP and tLNP comprise a sterol. Sterol refers to a subgroup of steroids that contain at least one hydroxyl (OH) group. More specifically, a gonane derivative with an OH group substituted for an H at position 3, or said differently, but equivalently, a steroid with an OH group substituted for an H at position 3. Examples of sterols include, without limitation, cholesterol, ergosterol, P-sitosterol, stigmasterol, stigmastanol, 20-hydroxycholesterol, 22-hydroxycholesterol, and the like. With respect to LNPs or tLNPs of this disclosure, in various embodiments, a sterol is cholesterol, 20-hydroxycholesterol, 22-hydroxycholesterol, or a phytosterol. In further embodiments, the phytosterol comprises campesterol, sitosterol, or stigmasterol, or combinations thereof. In preferred embodiments, the cholesterol is not animal-sourced but is obtained by synthesis using a plant sterol as a starting point. LNPs incorporating C-24 alkyl (such as methyl or ethyl) phytosterols have been reported to provide enhanced gene transfection. The length of the alkyl tail, the flexibility of the sterol ring, and polarity related to a retain C-3-OH group are important to obtaining high transfection efficiency. While s-sitosterol and stigmasterol performed well, vitamin D2, D3 and calcipotriol, (analogs lacking intact body of cholesterol) and betulin, lupeol ursolic acid and olenolic acid (comprising a 5^{th} ring) should be avoided. Sterols serve to fill space between other lipids in the LNP or tLNP and influence LNP or tLNP shape. Sterols also control fluidity of lipid compositions, reducing temperature dependence. Thus, sterols such as cholesterol, 20-hydroxycholesterol, 22-hydroxycholesterol, campesterol, fucosterol, P-sitosterol, and stigmasterol constitute means for controlling LNP shape and fluidity or sterol means for increasing transfection efficiency. In designing a lipid composition for a LNP or tLNP, in some embodiments, sterol content can be chosen to compensate for different amounts of other types of lipids, for example, ionizable cationic lipid or phospholipid.

In some embodiments, an LNP or tLNP has about 27 mol % or about 30 mol % to about 50 mol % sterol, or about 30 mol % to about 38 mol % sterol. In certain embodiments, an LNP or tLNP has about 30.5 mol %, about 33.5 mol %, or about 37.5 mol % sterol. In certain embodiments, an LNP or tLNP has 27 mol % or 30 mol % to 50 mol % sterol or 30 mol % to 38 mol % sterol. In further embodiments, an LNP or tLNP has 30.5 mol %, 33.5 mol %, or 37.5 mol % sterol. In certain instances, the sterol is cholesterol. In certain embodiments, the sterol is a mixture of sterols, for example, cholesterol and P-sitosterol or cholesterol and 20-hydroxycholesterol. In some instances, the sterol component is about 25 mol % 20-hydroxycholesterol and about 75 mol % cholesterol. In some instances, the sterol component is about 25 mol % s-sitosterol and about 75 mol % cholesterol. In some instances, the sterol component is about 50 mol % s-sitosterol and about 50 mol % cholesterol. In some instances, a sterol component is 25 mol % 20-hydroxycholesterol and 75 mol % cholesterol. In further instances, a sterol component is 25 mol % P-sitosterol and 75 mol % cholesterol. In still further instances, a sterol component is 50 mol % s-sitosterol and 50 mol % cholesterol.

### Co-Lipids

With respect to LNPs or tLNPs of this disclosure, in some embodiments, a co-lipid is absent or comprises an ionizable lipid, anionic or cationic. A co-lipid can be used to adjust various properties of an LNP or tLNP, such as surface charge, fluidity, rigidity, size, stability, and the like properties. In some embodiments, a co-lipid is an ionizable lipid, such as cholesterol hemisuccinate (CHEMS) or an ionizable lipid of this disclosure. In some embodiments, a co-lipid is a charged lipid, such as a quaternary ammonium headgroup containing lipid. In some embodiments, a quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 3β-(N—(N′,N′-Dimethylaminoethane)carbamoyl)cholesterol (DC-Chol), or combinations thereof. In certain embodiments, these compounds a chloride, bromide, mesylate, or tosylate salt. As described above, when quaternary ammonium headgroup containing lipids are included in LNPs or tLNPs, fatal hyperacute toxicity in laboratory animals has been observed. Accordingly, when the co-lipid is a quaternary ammonium headgroup containing lipid, the quaternary ammonium headgroup containing lipid is present it makes up no more than 50 mol % of the total cationic lipid, for example, from 5 to 50% of the total cationic lipid. For illustration, if an LNP or tLNP were to have cationic lipid content of 70 mol % and 5 to 50 mol % of the total cationic lipid as quaternary ammonium lipid, the LNP or tLNP would have from 3.5 mol % quaternary ammonium lipid and 66.5 mol % ionizable cationic lipid to 35 mol % each of quaternary ammonium lipid and ionizable cationic lipid.

When the disclosed ionizable lipids of formula M2 have a measured pKa between 6 and 7, they can contribute substantial endosomal release activity to an LNP or tLNP containing the ionizable lipid. More acidic or basic ionizable lipids of formula M2 can contribute surface charge and thus serve as a co-lipid as described immediately above. In such cases, it can be advantageous to incorporate another lipid with fusogenic activity into an LNP or tLNP of this disclosure. Surface charge is known to influence the tissue tropism of LNPs or tLNPs; for example, positively charged LNPs or tLNPs have shown a tropism for spleen and lung.

### PEG-Lipids

With respect to an LNP or tLNP of this disclosure, a PEG-lipid is a lipid conjugated to a polyethylene glycol (PEG). In some embodiments as described herein, the PEG-lipid is a C₁₄-C₂₀ lipid conjugated with a PEG. For example, in various embodiments as described herein, the PEG-lipid is a C₁₄-C₂₀ lipid conjugated with a PEG, or a C₁₄-C₁₈ lipid conjugated with a PEG, or a C₁₄-C₁₆ lipid conjugated with a PEG. In certain embodiments as described herein, the PEG-lipid is a fatty acid conjugated with a PEG. The fatty acid of the PEG-lipid can have a variety of chain lengths. For each, in some embodiments, the PEG-lipid is a fatty acid conjugated with PEG, wherein the fatty acid chain length is in the range of C₁₄-C₂₀ (e.g., in the range of C₁₄-C₁₈, or C₁₄-C₁₆). PEG-lipids with fatty acid chain lengths less than C₁₄ are too rapidly lost from the LNP of tLNP while those with chain lengths greater than C₂₀ are prone to difficulties with formulation.

PEG can be made in a large range of sizes. In certain embodiments, the PEG of the disclosed LNP and tLNP is PEG-1000 to PEG-5000. It is to be understood that polyethylene preparations of these sizes are polydisperse and that the nominal size indicates an approximate average molecular weight of the distribution. Taking the molecular weight of an individual repeating unit of (OCH2CH2)n to be 44, a PEG molecule with n=22 would have a molecular weight of 986, with n=45 a molecular weight of 1998, and with n=113 a molecular weight of 4990. n≈22 to 113 is used to represent PEG-lipids incorporating PEG moieties in the range of PEG-1000 to PEG-5000 such as PEG-1000, PEG-1500, PEG-2000, PEG-2500, PEG-3000, PEG-3500, PEG-4000, PEG-4500, and PEG-5000, although some molecules from preparations at the average molecular weight boundaries will have an n outside that range. For individual preparations n≈22 is used to represent PEG-lipids incorporating PEG moieties from PEG-1000, n≈45 is used to represent PEG-lipids incorporating PEG moieties from PEG-2000 n≈67 is used to represent PEG-lipids incorporating PEG moieties from PEG-3000, n≈90 is used to represent PEG-lipids incorporating PEG moieties from PEG-4000, n≈113 is used to represent PEG-lipids incorporating PEG moieties from PEG-5000. Some embodiments incorporate PEG moieties in a range bounded by any pair of the foregoing values of n or average molecular weight. In some embodiments of the PEG-lipid, a PEG is of 500-5000 or 1000-5000 Da molecular weight (MW). For example, in some embodiments, the PEG of the PEG-lipid has a molecular weight in the range of 1500-5000 Da or 2000-5000 Da. In some embodiments as described herein, the PEG-lipid has a molecular weight in the range of 500-4000 Da, or 500-3000 Da, or 1000-4000 Da, or 1000-3000, or 1000-2500, or 1500-4000, or 1500-3000, or 1500-2500 Da. In some embodiments, the PEG moiety is PEG-500, PEG-1000, PEG-1500, PEG-2000, PEG-2500, PEG-3000, PEG-3500, PEG-4000, PEG-4500, and PEG-5000. In some embodiments, the PEG unit has a MW of 2000 Da (sometime abbreviated as PEG(2k)). Some embodiments incorporate PEG moieties of PEG-1000, PEG-2000, or PEG-5000. In some instances, the PEG moiety is PEG-2000. Certain embodiments comprise a DSG-PEG, for example, DSG-PEG-2000. Certain embodiments comprise a DSPE-PEG, for example, DSPE-PEG-2000. Certain embodiments comprise both DSG-PEG-2000 and/or DSPE-PEG2000.

Common PEG-lipids fall into two classes diacyl glycerols and diacyl phospholipids. Examples of diacyl glycerol PEG-lipids include DMG-PEG (1,2-dimyristoyl-glycero-3-methoxypolyethylene glycol), DPG-PEG (1,2-dipalmitoyl-glycero-3-methoxypolyethylene glycol), DSG-PEG (1,2-distearoyl-glycero-3-methoxypolyethylene glycol), and DOG-PEG (1,2-dioleoyl-glycero-3-methoxypolyethylene glycol). Examples of diacyl phospholipids include DMPE-PEG (1,2-dimyristoyl-glycero-3-phosphoethanolamine-3-methoxypolyethylene glycol), DPPE-PEG (1,2-dipalmitoyl-glycero-3-phosphoethanolamine-3-methoxypolyethylene glycol), DSPE-PEG (1,2-distearoyl-glycero-3-phosphoethanolamine-3-methoxypolyethylene glycol), and DOPE-PEG (1,2-dioleoyl-glycero-3-phosphoethanolamine-3-methoxypolyethylene glycol).

In some embodiments, the MW2000 PEG-lipid (e.g., a PEG-lipid comprising a PEG of a molecular weight of 2000 Da) comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof. In some embodiments, the PEG unit has a MW of 2000 Da. In some embodiments, the MW2000 PEG-lipid comprises DMrG-PEG2000 (1,2-dimyristoyl-rac-glycero-3-methoxypolyethylene glycol-2000), DPrG-PEG2000 (1,2-dipalmitoyl-rac-glycero-3-methoxypolyethylene glycol-2000), DSrG-PEG2000 (1,2-distearoyl-rac-glycero-3-methoxypolyethylene glycol-2000), DorG-PEG2000 (1,2-dioleoyl-glycero-3-methoxypolyethylene-rac-glycol-2000), DMPEr-PEG200 (1,2-dimyristoyl-rac-glycero-3-phosphoethanolamine-3-methoxypolyethylene glycol-2000), DPPEr-PEG2000 (1,2-dipalmitoyl-rac-glycero-3-phosphoethanolamine-3-methoxypolyethylene glycol-2000), DSPEr-PEG2000 (1,2-distearoyl-rac-glycero-3-phosphoethanolamine-3-methoxypolyethylene glycol-2000), DOPEr-PEG2000 (1,2-dioleoyl-rac-glycero-3-phosphoethanolamine-3-methoxypolyethylene glycol-2000), or combinations thereof. The glycerol in these lipids is chiral. Thus, in some embodiments, the PEG-lipid is racemic. Alternatively, optically pure antipodes of the glycerol portion can be employed, that is, the glycerol portion is homochiral. As used herein with respect to glycerol moieties, optically pure means 295% of a single enantiomer (D or L). In some embodiments, the enantiomeric excess is 298%. In some embodiments, the enantiomeric excess is 299%. Additional PEG-lipids, including achiral PEG-lipids built on a symmetric dihydroxyacetone scaffold, a symmetric 2-(hydroxymethyl)butane-1,4-diol, or a symmetric glycerol scaffold, are disclosed in U.S. Provisional Application No. 63/362,502, filed on Apr. 5, 2022, and PCT/US2023/017648 filed on Apr. 5, 2023 (WO 2023/196445A1), both entitled PEG-Lipids and Lipid Nanoparticles, which are incorporated by reference in their entirety.

The above PEG-lipid examples are presented as methoxypolyethylene glycols, but the terminus need not necessarily be methoxyl. With respect to any of the PEG-lipids that have not been functionalized, in alternative embodiments, the PEG moiety of the PEG lipids can terminate with a methoxyl, a benzyloxyl, a 4-methoxybenzyloxyl, or a hydroxyl group (that is, an alcohol). The terminal hydroxyl facilitates functionalization. The methoxyl, benzyloxyl, and 4-methoxybenzyloxyl groups are advantageously provided for PEG-lipid that will be used as a component of the LNP without functionalization. However, all four of these alternatives are useful as the (non-functionalized) PEG-lipid component of LNPs. The 4-methoxybenzyloxyl group, often used as a protecting group during synthesis of the PEG-lipid, is readily removed to generate the corresponding hydroxyl group. Thus, the 4-methoxybenzyloxyl group offers a convenient path to the alcohol when it is not synthesized directly. The alcohol is useful for being functionalized, prior to incorporation of the PEG-lipid into a LNP, so that a binding moiety can be conjugated to it as a targeting moiety for the LNP (making it a tLNP). As used herein, the terminus of the PEG moiety, and similar constructions, refers to the end of the PEG moiety that is not attached to the lipid.

A PEG-moiety provides a hydrophilic surface on the LNP, inhibiting aggregation or merging of LNP, thus contributing to their stability and reducing polydispersity, i.e. reducing the heterogeneity of a dispersion of LNPs. Additionally, a PEG moiety can impede binding by the LNP, including binding to plasma proteins. These plasma proteins include apoE which is understood to mediate uptake of LNP by the liver so that inhibition of binding can lead to an increase in the proportion of LNP reaching other tissues. These plasma proteins also include opsonins so that inhibition of binding reduces recognition by the reticuloendothelial system. The PEG-moiety can also be functionalized to serve as an attachment point for a targeting moiety. Conjugating a cell- or tissue-specific binding moiety to the PEG-moiety enables a tLNP to avoid the liver and bind to its target tissue or cell type, greatly increasing the proportion of LNP that reaches the targeted tissue or cell type. PEG-lipid can thus serve as means for inhibiting LNP binding, and PEG-lipid conjugated to a binding moiety can serve as means for LNP-targeting.

As used herein, the term “functionalized PEG-lipid” and similar constructions refer generally to both the unreacted and reacted entities. The lipid composition of a LNP can be described referencing the reactive species even after conjugation has taken place (forming a tLNP). For example, a lipid composition can be described as comprising DSPE-PEG-maleimide and can be said to further comprise a binding moiety without explicitly noting that upon reaction to form the conjugate the maleimide will have been converted to a succinimide (or hydrolyzed succinimide). Similarly, if the reactive group is bromomaleimide, after conjugation it will be maleimide. These differences of chemical nomenclature for the unreacted and reacted species are to be implicitly understood even when not explicitly stated. Certain embodiments comprise a DSG-PEG, for example, DSG-PEG-2000. Certain embodiments comprise a functionalized DSPE-PEG, for example, functionalized DSPE-PEG-2000. Certain embodiments comprise both DSG-PEG-2000 and functionalized DSPE-PEG-2000. In some instances, the functionalized PEG-lipid is functionalized with a maleimide moiety, for example, DSPE-PEG-2000-MAL.

In certain aspects, the LNP comprises one or more PEG-lipids and/or functionalized PEG-lipids; when both a functionalized and unfunctionalized PEG-lipid, the PEG-lipid present they can be the same or different; and one or more ionizable cationic lipids; the LNP can further comprise a phospholipid, a sterol, a co-lipid, or any combination thereof. The term “functionalized PEG-lipid” refers to a PEG-lipid in which the PEG moiety has been derivatized with a chemically reactive group that can be used for conjugating a targeting moiety to the PEG-lipid. The functionalized PEG-lipid can be reacted with a binding moiety so that the binding moiety is conjugated to the PEG portion of the lipid. The conjugated binding moiety can thus serve as a targeting moiety for the LNP to constitute a tLNP. In some embodiments, the binding moiety is conjugated to the functionalized PEG-lipid after an LNP comprising the functionalized PEG-lipid is formed. In other embodiments, the binding moiety is conjugated to the PEG-lipid and then the conjugate is inserted into a previously formed LNP.

In certain embodiments, the LNP is a tLNP comprising one or more functionalized PEG-lipids that has been conjugated to a binding moiety. In certain embodiments, the tLNP also comprises PEG-lipids not functionalized or conjugated with a binding moiety. In some embodiments, the functionalization is a maleimide. In some embodiments the functionalization is a bromomaleimide or bromomaleimide amide, alkynylamide, or alkynylimide moiety at the terminal hydroxyl end of the PEG moiety. In some embodiments, the binding moiety comprises an antibody or antigen binding portion thereof. In some embodiments, the binding moiety is a polypeptide comprising a binding domain and an N- or C-terminal extension comprising an accessible thiol group. In some embodiments, the conjugation linkage comprises a reaction product of a thiol in the binding moiety with a functionalized PEG-lipid. In some embodiments, the functionalization is a maleimide, azide, alkyne, dibenzocyclooctyne (DBCO), bromomaleimide or bromomaleimide amide, alkynylamide, or alkynylimide. In some embodiments, the binding moiety comprises an antibody or antigen binding portion thereof. In some embodiments, the binding moiety is a polypeptide comprising a binding domain and an N- or C-terminal extension comprising an accessible thiol group.

In certain embodiments, the PEG-lipid and/or functionalized PEG-lipid comprises a scaffold selected from Formula S1, Formula S2, Formula S3, or Formula S4:

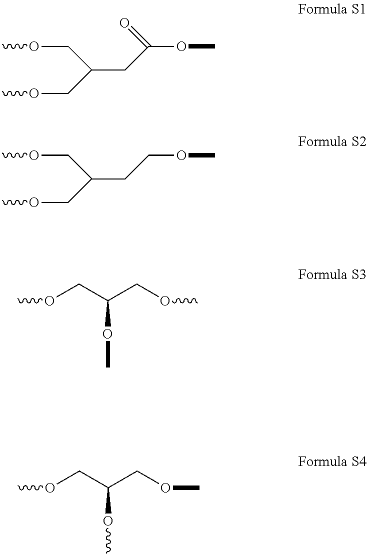

wherein [symbol] represents the points of ester connection with a fatty acid, and [symbol] represents the point of ester (S1) or ether (S2, S3, and S4) formation with the PEG moiety. In some embodiments, the fatty acid esters are C₁₄-C₂₀ straight-chain alkyl fatty acids. In some embodiments, the PEG moiety is functionalized and the fatty acid esters are C₁-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. By symmetric it is meant that each alkyl branch has the same number of carbons. In some embodiments, the branch is at the 3, 4, 5, 6, or 7 position of the fatty acid ester. The synthesis and use of PEG-lipids built on scaffolds S1-S4 is disclosed in WO2023/196445A1 which is incorporated by reference for all that it teaches about PEG-lipids and their use.

Some embodiments of the disclosed ionizable cationic lipids have head groups with small (<250 Da) PEG moieties. These lipids are not what is meant by the term PEG-lipid as used herein. These small PEG moieties are generally too small to impede binding to a similar extent as the larger PEG moieties of the PEG-lipids disclosed above, though they will impact the lipophilicity of ionizable cationic lipid. Moreover, the PEG-lipids are understood to be primarily located in an exterior facing lamella whereas much of the ionizable cationic lipid is in the interior of the LNP.

In certain embodiments, a functionalized PEG-lipid of a LNP (or tLNP) comprises one or more fatty acid tails, each that is no shorter than C₁₆ nor longer than C₂₀ for straight-chain fatty acids. For branched chain fatty acids, tails no shorter than C₁₄ fatty acids nor longer than C₂₀ are acceptable. In some embodiments, fatty acid tails are C₁₆. In some embodiments, the fatty acid tails are C₁₈. In some embodiments, the functionalized PEG-lipid comprises a dipalmitoyl lipid. In some embodiments, the functionalized PEG-lipid comprises a distearoyl lipid. The fatty acid tails serve as means to anchor the PEG-lipid in the tLNP to reduce or eliminate shedding of the PEG-lipid from the tLNP. This is a useful property for the PEG-lipid whether or not it is functionalized but has greater significance for the functionalized PEG-lipid as it will have a targeting moiety attached to it and the targeting function could be impaired if the PEG-lipid (with the conjugated binding moiety) were shed from the tLNP.

In some embodiments, an LNP or tLNP comprises about 0.5 mol % to about 3 mol % or 0.5 mol % to 3 mol % PEG-lipid comprising functionalized and non-functionalized PEG-lipid. In certain embodiments, an LNP or tLNP comprises DSG-PEG. In other embodiments, an LNP or tLNP comprises DMG-PEG or DPG-PEG. In certain embodiments, an LNP or tLNP comprises DSPE-PEG. In some embodiments, the functionalized and non-functionalized PEG-lipids are not the same PEG-lipid, for example, the non-functionalized PEG-lipid can be a diacylglycerol and the functionalized PEG-lipid a diacyl phospholipid. tLNP with such mixtures have reduced expression in the liver, possibly due to reduced uptake. In certain embodiments the functionalized PEG-lipid is DSPE-PEG and the non-functionalized PEG-lipid is DSG-PEG. In some embodiments, an LNP or tLNP comprises about 0.4 mol % to about 2.9 mol % or about 0.9 mol % to about 1.4 mol % non-functionalized PEG lipid. In certain embodiments, an LNP or tLNP comprises about 1.4 mol % or 1.4 mol % non-functionalized PEG lipid. In some embodiments, an LNP or tLNP comprises about 0.1 mol % to about 0.3 mol % or 0.1 mol % to 0.3 mol % functionalized lipid. In some instances, the functionalized lipid is DSPE-PEG. In certain instances, an LNP or tLNP comprises about 0.1 mol %, about 0.2 mol %, or about 0.3 mol % DSPE-PEG. In certain instances, an LNP or tLNP comprises 0.1 mol %, 0.2 mol %, or 0.3 mol % DSPE-PEG. In certain instances, the functionalized PEG-lipid is conjugated to a binding moiety. As used herein, the phrase “is conjugated to” and similar constructions are meant to convey a state of being, that is, a structure, and not a process, unless context dictates otherwise.

### Conjugation

Any suitable chemistry can be used to conjugate the binding moiety to the PEG of the PEG-lipid, including maleimide (see Parhiz et al., 2018, Journal of Controlled Release 291:106-115) and click (see Kolb et al., 2001, Angewandte Chemie International Edition 40(11):2004-2021; and Evans, 2007, Australian Journal of Chemistry 60(6):384-395) chemistries. Reagents for such reactions include lipid-PEG-maleimide, lipid-PEG-cysteine, lipid-PEG-alkyne, lipid, PEG-dibenzocyclooctyne (DBCO), and lipid-PEG-azide. Further conjugations reactions make use of lipid-PEG-bromo maleimide, lipid-PEG-alkylnoic amide, PEG-alkynoic imide, and lipid-PEG-alkyne reactions, as disclosed in U.S. Provisional Application No. 63/362,502, filed on Apr. 5, 2022, and PCT/US2023/017648 filed on Apr. 5, 2023 (WO 2023/196445A1), both entitled PEG-Lipids and Lipid Nanoparticles, which are incorporated by reference in their entirety. On the binding moiety side of the reaction can be used an existing cysteine sulfhydryl, or the protein derivatized by adding a sulfur containing carboxylic acid, for example, to the epsilon amino of a lysine to react with a maleimide, bromomaleimide, alkylnoic amide, or alkynoic imide. In certain embodiments, to modify an epsilon amino of a binding moiety lysine to react with a maleimide functionalized PEG-lipid, the binding moiety (e.g., an antibody) can be reacted with N-succinimidyl S-acetylthioacetate (SATA). SATA is then deprotected, for example, using 0.5 M hydroxylamine followed by removal of the unreacted components by G-25 Sephadex Quick Spin Protein columns (Roche Applied Science, Indianapolis, IN). The reactive sulfhydryl group on the binding moiety is then conjugated to maleimide moieties on LNPs of the disclosure using thioether conjugation chemistry. Alternatively, an alkyne can be added to a sulfhydryl or an epsilon amino of a lysine to participate in a click chemistry reaction.

Purification can be performed using Sepharose CL-4B gel filtration columns (Sigma-Aldrich). tLNPs (LNPs conjugated with a targeting antibody) can be stored frozen, for example at −70 or −80° C. until needed. Others have conjugated antibody to free functionalized PEG-lipid and then incorporated the conjugated lipid into pre-formed LNP. However, it was found that the present procedure is more controllable and produces more consistent results.

There are also several approaches to site-specific conjugation. Particularly but not exclusively suitable for truncated forms of antibody, C-terminal extensions of native or artificial sequences containing a particularly accessible cysteine residue are commonly used. Partial reduction of cysteine bonds in an antibody, for example, with tris(2-carboxy)phosphine (TCEP), can also generate thiol groups for conjugation which can be site-specific under defined conditions with an amenable antibody fragment. Alternatively, the C-terminal extension can contain a sortase A substrate sequence, LPXTG (SEQ ID NO: 6) which can then be functionalized in a reaction catalyzed by sortase A and conjugated to the PEG-lipid, including through click chemistry reactions (see, for example, Moliner-Morro et al., Biomolecules 10(12):1661, 2020 which is incorporated by reference herein for all that it teaches about antibody conjugations mediated by the sortase A reaction and/or click chemistry). The use of click chemistry for the conjugation of a targeting moiety, such as various forms of antibody, is disclosed, for example, in WO2024/102,770 which is incorporated by reference in its entirety for all that it teaches about the conjugation of targeting moieties to LNPs that is not inconsistent with this disclosure.

For whole antibody and other forms comprising an Fc region, site-specific conjugation to either (or both) of two specific lysine residues (Lys248 and Lys288) can be accomplished without any change to or extension of the native antibody sequence by use of one of the AJICAP® reagents (see, for example, Matsuda et al., Mol. Pharmaceutics 18:4058, 2021 and Fujii et al., Bioconjugate Chem. 34:728, 2023, which are incorporated by reference herein for all that they teach regarding conjugation of antibodies with AJICAP reagents). AJICAP reagents are modified affinity peptides that bind to specific loci on the Fc and react with an adjacent lysine residue to form an affinity peptide conjugate of the antibody. The peptide is then cleaved with base to leave behind a thiol-functionalized lysine residue which can then undergo conjugation through maleimide or haloamide reactions, for example). Functionalization with azide or dibenzocyclooctyne (DBCO) for conjugation by click chemistry is also possible. This and similar technology are further described in US 2020/0190165 (corresponding to WO 2018/199337), US 2021/0139549 (corresponding to WO 2019/240287) and US 2023/0248842 (corresponding to WO 2020/184944) which are incorporated by reference in their entirety for all that they teach about such modified affinity peptides and their use.

Accordingly, in some embodiments the binding moiety is conjugated to the PEG moiety of the PEG-lipid through a thiol modified lysine residue. In some embodiments, the conjugation is through a cysteine residue in a native or added antibody sequence. In some embodiments, a particular cysteine residue is preferentially or exclusively reacted, for example, a cysteine residue in an antibody hinge region. In further instances, a binding moiety with a conjugatable cysteine residue in an antibody hinge region is an Fab′ or similar fragment. In other embodiments, the conjugation is through a sortase A substrate sequence. In still other embodiments, the conjugation is through a specific lysine residue (Lys248 or Lys288) in the Fc region.

### Binding Moieties

The tLNP of the various disclosed aspects comprise a binding moiety, such as an antibody or antigen binding domain thereof or a cell surface receptor ligand. As used herein, a “binding moiety” or “targeting moiety” refers to a protein, polypeptide, oligopeptide or peptide, carbohydrate, nucleic acid, or combinations thereof capable of specifically binding to a target or multiple targets. A binding domain includes any naturally occurring, synthetic, semi-synthetic, or recombinantly produced binding partner for a biological molecule or another target of interest. Exemplary binding moieties of this disclosure include an antibody, a Fab′, F(ab′)₂, Fab, Fv, rigG, scFv, hcAb (heavy chain antibody), a single domain antibody, VHH, VNAR, sdAb, nanobody, receptor ectodomain or ligand-binding portions thereof, or ligand (e.g., cytokines, chemokines). An “Fab” (antigen binding fragment) is the part of an antibody that binds to antigens and includes the variable region and CH1 of the heavy chain linked to the light chain via an inter-chain disulfide bond. In other embodiments, a binding moiety comprises a ligand-binding domain of a receptor or a receptor ligand. In some embodiments, a binding moiety can have more than one specificity including, for example, bispecific or multispecific binders. A variety of assays are known for identifying binding moieties of this disclosure that specifically bind a particular target, including Western blot, ELISA, biolayer interferometry, and surface plasmon resonance. A binding moiety, such as a binding moiety comprising immunoglobulin light and heavy chain variable domains (e.g., scFv), can be incorporated into a variety of protein scaffolds or structures as described herein, such as an antibody or an antigen binding fragment thereof, a scFv-Fc fusion protein, or a fusion protein comprising two or more of such immunoglobulin binding domains.

The fundamental ability of the tLNP to deliver a payload into the cytoplasm of a cell is agnostic with respect to, and does not depend upon, a particular binding specificity. Of course, a binding moiety is a determinant of which cells a payload is delivered into. There are many known antibodies with specificity for one or another cell surface marker associated with particular cell type(s) that could be used as the target of the binding moiety on a disclosed tLNP and there are several sources that have compiled such information. An excellent source of information about antibodies for which an International Non-proprietary Name (INN) has been proposed or recommended is Wilkinson & Hale, MAbs 14(1):2123299, 2022, including its Supplementary Tables, which is incorporated by reference herein for all that it teaches about individual antibodies and the various antibody formats that can be constructed. U.S. Pat. No. 11,326,182 and especially its Table 9 Cancer, Inflammation and Immune System Antibodies, is a source of sequence and other information for a wide range of antibodies including many that do not have an INN and is incorporated herein by reference for all that it teaches about individual antibodies. Sequence information is not always readily available for antibodies mentioned in the art, even when commercially available. This is not necessarily an impediment to their use. Where the antibody or a cell line is commercially available or obtainable from its originator it can be used as the binding moiety of tLNP without any need for sequence information. Even where sequence information is needed, it is well within the capabilities of the skilled artisan to sequence the antibody protein (or have it done by a contract laboratory) so that the antibody's variable region can be incorporated into a scFv, a diabody, a minibody, or some other antibody format, or be humanized. In choosing among available antibodies in the art for the development of an agent to be used in humans, a human antibody is preferred to a humanized antibody is preferred to a non-human antibody, other factors being equal. Other factors can include stability and ease of production of the antibody, affinity of the antibody, lack of binding to non-target extracellular and cell surface antigens, and cross-reactivity for the cognate antigen in model species to be used in product development.

In some embodiments, a binding moiety can be an antibody or an antigen-binding portion thereof; an antigen; a ligand-binding domain of a receptor; or a receptor ligand. In some embodiments, a binding moiety can have more than one specificity including, for example, bispecific or multispecific binders.

In some embodiments, a binding moiety comprises an antibody or an antigen-binding portion thereof. As used herein, “antibody” refers to a protein comprising an immunoglobulin domain having hypervariable regions determining the specificity with which the antibody binds antigen, termed complementarity determining regions (CDRs). The term antibody can thus refer to intact (i.e., whole) antibodies as well as antibody fragments and constructs comprising an antigen binding portion of a whole antibody. While the canonical natural antibody has a pair of heavy and light chains, camelids (camels, alpacas, llamas, etc.) produce antibodies with both the canonical structure and antibodies comprising only heavy chains. The variable region of the camelid heavy chain-only antibody has a distinct structure with a lengthened CDR3 referred to as VHH or, when produced as a fragment, a nanobody. Antigen binding fragments and constructs of antibodies include F(ab′)₂, F(ab′), F(ab), minibodies, Fv, single-chain Fv (scFv), diabodies, and VH. Such elements can be combined to produce bi- and multi-specific reagents, including various immune cell engagers, such as BiTEs (bi-specific T-cell engagers). The term “monoclonal antibody” arose out of hybridoma technology but is now used to refer to any singular molecular species of antibody regardless of how it was originated or produced. Antibodies can be obtained through immunization, selection from a naïve or immunized library (for example, by phage display), alteration of an isolated antibody-encoding sequence, or any combination thereof. Numerous antibodies that can be used as binding moieties are known in the art. An excellent source of information about antibodies for an International Non-proprietary Name (INN) has been proposed or recommended, including sequence information, is Wilkinson & Hale, 2022, MAbs 14(1):2123299, including its Supplementary Tables, which is incorporated by reference herein for all that it teaches about individual antibodies and the various antibody formats that can be constructed. U.S. Pat. No. 11,326,182 and especially its Table 9 entitled “Cancer, Inflammation and Immune System Antibodies,” is a source of sequence and other information for a wide range of antibodies including many that do not have an INN and is incorporated herein by reference for all that it teaches about individual antibodies and the antigens they bind. WO02024040195A1 is also a source of sequence and other information for a wide range of antibodies with specificity for various cell surface antigens of immune system cells and cancer cells and is incorporated herein by reference for all that it teaches about individual antibodies and the antigens they bind.

An antibody or other binding moiety (or a fusion protein thereof) “specifically binds” a target if it binds the target with an affinity or Ka (i.e., an equilibrium association constant of a particular binding interaction with units of 1/Molar or 1/M) equal to or greater than 10⁵ M⁻¹, while not significantly binding other components present in a test sample. Binding domains (or fusion proteins thereof) can be classified as “high affinity” binding domains (or fusion proteins thereof) and “low affinity” binding domains (or fusion proteins thereof). “High affinity” binding domains refer to those binding domains with a Ka of at least 10⁸ M⁻¹, at least 108 M⁻¹, at least 10¹⁰ M⁻¹, at least 10¹¹ M⁻¹, at least 10¹2 M⁻¹, or at least 10¹³ M⁻¹, preferably at least 10′ M⁻¹ or at least 10⁹ M⁻¹. “Low affinity” binding domains refer to those binding domains with a Ka of up to 10⁸ M⁻¹, up to 107 M⁻¹, up to 108 M⁻¹, up to 10⁵ M⁻¹. Alternatively, affinity can be defined as an equilibrium dissociation constant (Kd) of a particular binding interaction with units of M (e.g., 10⁻⁵ M to 10⁻¹³ M). Affinities of binding domain polypeptides and fusion proteins according to this disclosure can be readily determined using conventional techniques (see, e.g., Scatchard et al., 1949, Ann. N.Y. Acad. Sci. 51:660; and U.S. Pat. Nos. 5,283,173, 5,468,614, or the equivalent).

A diabody is a type of scFv dimer in which each chain consists of the V_{H} and V_{L} regions connected by a small peptide linker that is too short to allow pairing between the two domains of the same chain. This arrangement forces the V_{H} of one chain to pair with the V_{L} of a second chain thereby forming a bivalent, and often bispecific, dimer. A BiTE is a fusion protein having two scFvs of different antibodies, usually an antibody for a tumor-associated antigen and antibody for CD3, on a single peptide chain, thus forming a cytolytic synapse between T cells and target antigen-bearing cells. The term “antigen-binding portion” can refer to a portion of an antibody as described that possesses the ability to specifically recognize, associate, unite, or combine with a target molecule. An antigen-binding portion includes any naturally occurring, synthetic, semi-synthetic, or recombinantly produced binding partner for a specific antigen. Thus, antibodies and antigen-binding portions thereof constitute means for binding to the surface molecule on a cell. In various embodiments, the cell can be an immune cell, a leukocyte, a lymphocyte, a monocyte, a stem cell, an HSC or an MSC, according to the specificity of the antibody.

In some embodiments, the antibody or antigen-binding portion thereof can be derived from a mammalian species, for example, mice, rats, or human. Antibody variable regions can be those arising from one species, or they can be chimeric, containing segments of multiple species possibly further altered to optimize characteristics such as binding affinity or low immunogenicity. For human applications, it is desirable that the antibody has a human sequence. In the cases where the antibody or antigen-binding portion thereof is derived from a non-human species, the antibody or antigen-binding portion thereof can be humanized to reduce immunogenicity in a human subject. For example, if a human antibody of the desired specificity is not available, but such an antibody from a non-human species is, the non-human antibody can be humanized, e.g., through CDR grafting, in which the CDRs from the non-human antibody are placed into the respective positions in a framework of a compatible human antibody. Less preferred is an antibody in which only the constant region of the non-human antibody is replaced with human sequence. Such antibodies are commonly referred to as chimeric antibodies in distinction to humanized antibodies.

In some embodiments, the antibody or antigen-binding portion thereof is non-immunogenic. In some embodiments, the antibody can be modified in its Fc region to reduce or eliminate secondary functions, such as FcR engagement, antibody-dependent cellular cytotoxicity (ADCC), antibody-dependent cellular phagocytosis (ADCP), and/or complement-dependent cytotoxicity (CDC); this is often referred to as an Fc silenced antibody.

A binding moiety density on the LNP (or tLNP) can be defined according to the ratio of antibody (binder) to mRNA (w/w) either based on the amount of antibody input in the conjugation reaction or as measured in the LNP or (tLNP) after conjugation. For an intact antibody (e.g., whole IgG), in some embodiments, preferred ratios are about 0.3 to about 1.0, about 0.3 to about 0.7, about 0.3 to about 0.5, about 0.5 to about 1.0, and about 0.5 to about 0.7 for either the input or final measured binder ratio. In certain embodiments, a LNP (or tLNP) has an antibody ratio of 0.3 to 1.0, 0.3 to 0.7, 0.3 to 0.5, 0.5 to 1.0, and 0.5 to 0.7 for either the input or final measured binder ratio. In some embodiments, if the binder is different in size from an intact antibody (for example a scFv, diabody, or minibody, etc.) the w/w ratio is adjusted for the different size of the binder.

In certain embodiments, a LNP (or tLNP) comprises a binding moiety derived from an anti-CD40*^{‡} antibody, an anti-LRRC15^{†‡} antibody, an anti-CTSK antibody, an anti-ADAM12^{‡} antibody, an anti-ITGA11 antibody, an anti-FAP*^{†‡} antibody, an anti-NOX4 antibody, an anti-SGCD antibody, an anti-SYNDIG1 antibody, an anti-CDH11^{‡} antibody, an anti-PLPP4 antibody, an anti-SLC24A2 antibody, an anti-PDGFRB*^{‡} antibody, an anti-THY1^{‡} antibody, an anti-ANTXR1^{‡} antibody, an anti-GAS1 antibody, an anti-CALHM5 antibody, an anti-SDC1*^{‡} antibody, an anti-HER2*^{†‡} antibody, an anti-TROP2*^{†‡} antibody, an anti-MSLN*^{‡} antibody, an anti-Nectin4^{†‡} antibody, or an anti-MUC16*^{†‡} antibody. In further embodiments, a LNP (or tLNP) comprises a binding moiety specific for an immune cell antigen selected from CD1, CD2*^{†‡}, CD3*^{†‡}, CD4*^{†‡}*, CD5^{†‡}, CD7^{†‡}, CD8^{‡}, CD11b^{‡}, CD14^{†‡}, CD16, CD25^{†‡}, CD26*^{‡}, CD27*^{†‡}, CD28*^{†‡}, CD30*^{†‡}, CD32*, CD38*^{†‡}*, CD39^{‡}, CD40*^{†‡}*, CD40L (CD154)*^{†‡}, CD44*^{‡}, CD45^{†‡}, CD64*^{†‡}, CD62^{†‡}, CD68, CD69^{‡}, CD73^{†‡}, CD80*^{‡}, CD83^{‡}, CD86*^{‡}, CD95^{‡}, CD103^{‡}, CD119^{‡}, CD126^{‡}, CD137 (4-1BB)^{†‡}, CD150^{‡}, CD153^{‡}, CD161^{‡}, CD166^{‡}, CD183 (CXCR3)^{‡}, CD183 (CXCR5)^{‡}, CD223 (LAG-3)*^{†‡}, CD254^{‡}, CD275^{‡}, CD45RA, CTLA-4*^{†}*^{†}, DEC205, OX40^{†}, PD-1*^{†‡}, GITR^{†}, TIM-3*^{†‡}, FasL*^{‡}, IL18R1, ICOS (CD278)^{‡}, leu-12, TCR†, TLR1, TLR2^{†‡}, TLR3*^{‡}, TLR4^{†‡}, TLR6, TREM2^{‡}, NKG2D^{‡}, CCR, CCR1 (CD191)^{‡}, CCR2 (CD192)*^{†‡}*, CCR4(CD194)*^{†‡}*, CCR6(CD196)^{‡}, CCR7^{‡}, low affinity IL-2 receptor^{†‡}, IL-7 receptor^{‡}, IL-12 receptor^{‡}, IL-15 receptor^{‡}, IL-18 receptor^{‡}, and IL-21 receptor^{‡}. In further embodiments, a tLNP comprises a binding moiety specific for an HSC surface molecule selected from CD117^{†}, CD34*^{‡}, CD44*^{‡}, CD90 (Thy1)^{‡}, CD105^{‡}, CD133^{‡}, BMPR2^{‡}, and Sca-1; or specific for an MSC surface molecules selected from CD70*^{‡}, CD105^{‡}, CD73^{‡}, Stro-1^{‡}, SSEA-3^{‡}, SSEA-4^{‡}, CD271^{‡}, CD146^{‡}, GD2^{†‡}, SUSD2, Stro-4, MSCA-1, CD56^{‡}, CD200*^{‡}, PODXL^{‡}, CD13^{‡}, CD29*^{‡}, CD44*^{‡}, and CD10^{‡}. In various embodiments, a binding moiety is an antibody or antigen-binding portion thereof. (* indicates that exemplary antibodies with the indicated specificity from which a binding moiety could be derived can be found in U.S. Pat. No. 11,326,182B2 Table 9 or 10. ^{†} indicates that exemplary antibodies with the indicated specificity from which a binding moiety could be derived can be found in Wilkinson & Hale, 2022. Both references cited and incorporated by reference above. ^{‡} indicates that exemplary antibodies with the indicated specificity from which a binding moiety could be derived can be found in the Therapeutic Antibody Database (TABS) at tabs.craic.com.). Other suitable antibodies can be found in Appendix A or WO2024040195A1 each of which is incorporated herein by reference for all that it teaches about individual antibodies and the antigens they bind.

The following paragraphs provide non-exhaustive examples of known antibodies that bind to cell surface markers/antigens on immune cells (lymphocytes and monocytes) and stem cells (HSC and MSC). These antibodies or the antigen binding domains thereof can be used as binding moieties to target the disclosed LNP. Similarly, these antibodies can contribute their antigen binding domains to immune cell reprogramming agents such as CARs and ICEs. While typically an immune cell reprogramming agent is expressed in an immune cell, one call also express a biological response modifier (conditioning agent) or an immune cell reprogramming agent, such as an ICE, in a tumor cell. The immune and stem cell surface markers that can serve as a targeted antigen of a tLNP can also usefully be a target of an immune cell reprogramming agent when the cell expressing that antigen has a role in the pathology of some disease or condition. Collectively these antibodies and polypeptides comprising the antigen binding domains thereof constitute means for binding cell surface markers or means for binding immune and stem cells.

In some embodiments, CD2 is a targeted cell surface antigen and a binding moiety comprises the antigen binding domain of an anti-CD2 antibody. CD2 contains three well-characterized epitopes (T11.1, T11.2, and T11.3/CD2R). T11.3/CD2R are membrane proximal and exposure is increased upon T cell activation and CD2 clustering. Accordingly, in some such embodiments, the anti-CD2 antigen binding domain is derived from, RPA-2.10; OKT11, UMCD2, 0.1, and 3T4-8B5 (T11.1 epitope); 9.6 and 10LD2-4C1 (T11.2 epitope); 1Mono2A6 (T11.3 epitope), siplizumab (T11.2/T11.3 epitope), HuMCD2, TS2/18, TS1/8, AB75, LT-2, T6.3, MEM-65, OT14E4, or an antigen-binding portion thereof. Additionally, the ligand of CD2, CD58 (LFA-3) can be used as a CD2 binding moiety as can alefacept, a CD58-Fc fusion. Each of these constitutes a means for binding CD2 (Li et al., 1996, J Mol Biol. 263:209-26; Binder et al., 2020, Front Immunol. 9:11:1090).

In some embodiments, CD3 is a targeted cell surface antigen and a binding moiety comprises the antigen binding domain of an anti-CD3 antibody. Accordingly, in some such embodiments, the antigen binding domain is derived from muromonab-CD3 (OKT3), teplizumab, otelixizumab, visilizumab, cevostamab, teclistamab, elranatamab pavurutamab, vibecotamab, odronextamab, or an antigen-binding portion thereof. Each of these constitutes a means for binding CD3.

In some embodiments, CD4 is a targeted cell surface antigen and a binding moiety comprises the antigen binding domain of an anti-CD4 antibody. Accordingly, in some such embodiments, the antigen binding domain is derived from ibalizumab, inezetamab, semzuvolimab, zanolimumab, tregalizumab, UB-421, priliximab, MTRX1011A, cedelizumab, clenoliximab, keliximab, M-T413, TRX1, hB-F5, MAX.16H5, IT208, or an antigen-binding portion thereof. Each of these constitutes a means for binding CD4.

In some embodiments, CD5 is a targeted cell surface antigen and a binding moiety comprises the antigen binding domain of an anti-CD5 antibody. Accordingly, in some such embodiments, the antigen binding domain is derived from 5D7, UCHT2, L17F12, H65, HE3, OKT1, MAT304, as well as those disclosed in WO1989006968, WO2008121160, U.S. Pat. No. 8,679,500, WO2010022737, WO2019108863, WO2022040608, or WO2022127844, each of which is incorporated by reference for all that they teach about anti-CD5 antibodies and their properties, or an antigen-binding portion thereof. Each of these constitutes a means for binding CD5.

In some embodiments, CD7 is a targeted cell surface antigen and a binding moiety comprises the antigen binding domain of an anti-CD7 antibody. Accordingly, in some such embodiments, the antigen binding domain is derived from TH-69, 3A1E, 3A1F, Huly-m2, WT1, YTH3.2.6, T3-3A1, grisnilimab, as well as those disclosed in U.S. Pat. No. 10,106,609, WO2017213979, WO2018098306, U.S. Ser. No. 11/447,548, WO2022136888, WO2020212710, WO2021160267, WO2022095802, WO2022095803, WO2022151851, or WO2022257835 each of which is incorporated by reference for all that they teach about anti-CD7 antibodies and their properties, or an antigen-binding portion thereof. Each of these constitutes a means for binding CD7.

In some embodiments, CD8 is a targeted cell surface antigen and a binding moiety comprises the antigen binding domain of an anti-CD8 antibody. Accordingly, in some such embodiments, the antigen binding domain is derived from crefmirlimab (IAB22M), 3B5, SP-16, LT8, 17D8, MEM-31, MEM-87, RIV11, UCHT4, YTC182.20, RPA-T8, OKT8, SK1, 51.1, TRX2, MT807-R1, HIT8α, C8/144B, RAVB3, SIDI8BEE, BU88, EPR26538-16, 2ST8.5H7, as well as those disclosed in U.S. Pat. No. 10,414,820, WO2015184203, WO2017134306, WO2019032661, WO2020060924, U.S. Pat. No. 10,730,944, WO2019033043, WO2021046159, WO2021127088, WO2022081516, U.S. Pat. No. 11,535,869, or WO2023004304 each of which is incorporated by reference for all that they teach about anti-CD8 antibodies and their properties, or an antigen-binding portion thereof. Additionally, humanized anti-CD8 antibodies are described in U.S. Provisional Patent Application No. 63/610,917, filed on Dec. 15, 2023, and U.S. Provisional Patent Application Number (Atty Docket No. 23-1742-US-PRO2), filed on May 31, 2024, each of which is incorporated by reference for all that it teaches about these humanized anti-CD8 antibodies and their properties, or an antigen-binding portion thereof. Each of the foregoing anti-CD8 antibodies constitutes a means for binding CD8.

In some embodiments, a tLNP is targeted to CD10 is a targeted cell surface antigen and a binding moiety comprises the antigen binding domain of an anti-CD10 antibody. Accordingly, in some such embodiments, the antigen binding domain is derived from the one produced by the hybridoma represented by Accession No. NITE BP-02489 (disclosed in WO02018235247 which is incorporated by reference for all that they teach about anti-CD10 antibodies and their properties), FR4D11, or REA877, or an antigen-binding portion thereof. Each of these constitutes a means for binding CD10.

In some embodiments, CD11b is a targeted cell surface antigen and a binding moiety comprises the antigen binding domain of an anti-CD11b antibody. Accordingly, in some such embodiments, the antigen binding domain is derived from ASD141 or MAB107 as well as those disclosed in US20150337039, U.S. Pat. No. 10,738,121, WO2016197974, U.S. Pat. No. 10,919,967, or WO2022147338 each of which is incorporated by reference for all that they teach about anti-CD11b antibodies and their properties, or an antigen-binding portion thereof. Each of these constitutes a means for binding CD11b.

In some embodiments, CD13 is a targeted cell surface antigen and a binding moiety comprises the antigen binding domain of an anti-CD13 antibody. CD13 is also known as aminopeptidase N (APN). Accordingly, in some such embodiments, the antigen binding domain is derived from MT95-4 or Nbl57 (disclosed in WO2021072312 which is incorporated by reference for all that they teach about anti-CD13 antibodies and their properties), as well as those disclosed in WO2023037015 which is incorporated by reference for all that it teaches about anti-CD13 antibodies and their properties, or an antigen-binding portion thereof. Each of these constitutes a means for binding CD13.

In some embodiments, CD14 is a targeted cell surface antigen and a binding moiety comprises the antigen binding domain of an anti-CD14 antibody. Accordingly, in some such embodiments, the antigen binding domain is derived from atibuclimab or r18D11 as well as those disclosed in WO2018191786 or WO2015140591 each of which is incorporated by reference for all that they teach about anti-CD14 antibodies and their properties, or an antigen-binding portion thereof. Each of these constitutes a means for binding CD14.

In some embodiments, CD16a is a targeted cell surface antigen and a binding moiety comprises the antigen binding domain of an anti-CD16a antibody. Accordingly, in some such embodiments, the antigen binding domain is derived from AFM13, sdA1, sdA2, or hu3G8-5.1-N297Q as well as those disclosed in U.S. Ser. No. 11/535,672, WO2018158349, WO2007009065, U.S. Ser. No. 10/385,137, WO2017064221, U.S. Pat. No. 10,758,625, WO2018039626, WO2018152516, WO2021076564, WO2022161314, or WO2023274183 each of which is incorporated by reference for all that they teach about anti-CD16A antibodies and their properties, or an antigen-binding portion thereof. Each of these constitutes a means for binding CD16a.

In some embodiments, CD25 is a targeted cell surface antigen and a binding moiety comprises the antigen binding domain of an anti-CD25 antibody. Accordingly, in some such embodiments, the antigen binding domain is derived from daclizumab, basiliximab, camidanlumab, tesirine, inolimomab, R07296682, HuMax-TAC, CYT-91000, STI-003, RTX-003, or an antigen-binding portion thereof. Each of these constitutes a means for binding CD25.

In some embodiments, CD28 is a targeted cell surface antigen and a binding moiety comprises the antigen binding domain of an anti-CD28 antibody. Accordingly, in some such embodiments, the antigen binding domain is derived from GN1412, acazicolcept, lulizumab, prezalumab, theralizumab, FR104CD, and davoceticept, as well as those disclosed in U.S. Pat. Nos. 8,454,959, 8,785,604, 11,548,947, 11,530,268, 11,453,721, 11,591,401, WO2002030459, WO2002047721, US20170335016, US20200181260, U.S. Ser. No. 11/608,376, WO2020127618, WO2021155071, or WO2022056199 each of which is incorporated by reference for all that they teach about anti-CD28 antibodies and their properties, or an antigen-binding portion thereof. Each of these constitutes a means for binding CD28.

In some embodiments, CD29 is a targeted cell surface antigen and a binding moiety comprises the antigen binding domain of an anti-CD29 antibody. Accordingly, in some such embodiments, the antigen binding domain is derived from OS2966, 6D276, 12G10, REA1060, or an antigen-binding portion thereof. Each of these constitutes a means for binding CD29.

In some embodiments, CD32A is a targeted cell surface antigen and a binding moiety comprises the antigen binding domain of an anti-CD32A antibody. Accordingly, in some such embodiments, the antigen binding domain is derived from VIB9600, humanized IV.3, humanized AT-10, or MDE-8 as well as those disclosed in U.S. Pat. Nos. 9,688,755, 9,284,375, 9,382,321, U.S. Ser. No. 11/306,145, or WO2022067394 each of which is incorporated by reference for all that they teach about anti-CD32A antibodies and their properties, or an antigen-binding portion thereof. Each of these constitutes a means for binding CD32A.

In some embodiments, CD34 is a targeted cell surface antigen and a binding moiety comprises the antigen binding domain of an anti-CD34 antibody. Accordingly, in some such embodiments, the antigen binding domain is derived from h4C8, 9C5, 2E10, 5B12, REA1164, C5B12, C2e10, or an antigen-binding portion thereof. Each of these constitutes a means for binding CD34.

In some embodiments, CD40 is a targeted cell surface antigen and a binding moiety comprises the antigen binding domain of an anti-CD40 antibody. Accordingly, in some such embodiments, the antigen binding domain is derived from cifurtilimab, sotigalimab, iscalimab, dacetuzumab, selicrelumab, bleselumab, lucatumumab, or mitazalimab as well as those disclosed in U.S. Ser. No. 10/633,444, each of which is incorporated by reference for all that they teach about anti-CD40 antibodies and their properties, or an antigen-binding portion thereof. Each of these constitutes a means for binding CD40.

In some embodiments, CD44 is a targeted cell surface antigen and a binding moiety comprises the antigen binding domain of an anti-CD44 antibody. Accordingly, in some such embodiments, the antigen binding domain is derived from R05429083, VB6-008, PF-03475952, or RG7356, as well as those disclosed in WO2008144890, U.S. Pat. No. 8,383,117, WO2008079246, US20100040540, WO2015076425, U.S. Pat. No. 9,220,772, US20140308301, WO2020159754, WO2021160269, WO2021178896, WO2022022749, WO2022022720, or WO2022243838, each of which is incorporated by reference for all that they teach about anti-CD44 antibodies and their properties, or an antigen-binding portion thereof. Each of these constitutes a means for binding CD44.

In some embodiments, CD45 is a targeted cell surface antigen and a binding moiety comprises the antigen binding domain of an anti-CD45 antibody. Accordingly, in some such embodiments, the antigen binding domain is derived from apamistamab, BC8-B10, as well as those disclosed in WO2023183927, WO2023235772, U.S. Pat. No. 7,825,222, WO2017009473, WO2021186056, U.S. Pat. Nos. 9,701,756, 9,701,756, WO2020092654, WO2022040088, WO2022040577, WO2022064191, WO2022063853, or WO2024064771, each of which is incorporated by reference for all that they teach about anti-CD45 antibodies and their properties, or an antigen-binding portion thereof. Each of these constitutes a means for binding CD45.

In some embodiments, CD56 is a targeted cell surface antigen and a binding moiety comprises the antigen binding domain of an anti-CD56 antibody. Accordingly, in some such embodiments, the antigen binding domain is derived from lorvotuzumab, adcitmer, or promiximab, as well as those disclosed in WO2012138537, U.S. Pat. Nos. 10,548,987, 10,730,941, or US20230144142, each of which is incorporated by reference for all that they teach about anti-CD56 antibodies and their properties, or an antigen-binding portion thereof. Each of these constitutes a means for binding CD56.

In some embodiments, CD64 is a targeted cell surface antigen and a binding moiety comprises the antigen binding domain of an anti-CD64 antibody. Accordingly, in some such embodiments, the antigen binding domain is derived from HuMAb 611 or H22 as well as those disclosed in U.S. Pat. No. 7,378,504, WO2014083379, US20170166638, or WO2022155608 each of which is incorporated by reference for all that they teach about anti-CD64 antibodies and their properties, or an antigen-binding portion thereof. Each of these constitutes a means for binding CD64.

In some embodiments, CD68 is a targeted cell surface antigen and a binding moiety comprises the antigen binding domain of an anti-CD68 antibody. Accordingly, in some such embodiments, the antigen binding domain is derived from Ki-M7, PG-M1, 514H12, ABM53F5, 3F7C6, 3F7D3, Y1/82A, EPR20545, CDLA68-1, LAMP4-824, or an antigen-binding portion thereof. Each of these constitutes a means for binding CD68.

In some embodiments, CD70 is a targeted cell surface antigen and a binding moiety comprises the antigen binding domain of an anti-CD70 antibody. Accordingly, in some such embodiments, the antigen binding domain is derived from cusatuzumab, vorsetuzumab, MDX-1203, MDX-1411, AMG-172, SGN-CD70A, ARX305, PRO1160, as well as those disclosed in U.S. Pat. Nos. 9,765,148, 8,124,738, IS10,266,604, WO2021138264, U.S. Pat. Nos. 9,701,752, 10,108,123, WO2014158821, U.S. Pat. No. 10,689,456, WO2017062271, U.S. Pat. Nos. 11,046,775, 11,377,500, WO2021055437, WO2021245603, WO2022002019, WO2022078344, WO2022105914, WO2022143951, WO2023278520, WO2022226317, WO2022262101, U.S. Pat. No. 11,613,584, or WO2023072307, each of which is incorporated by reference for all that they teach about anti-CD70 antibodies and their properties, or an antigen-binding portion thereof. Each of these constitutes a means for binding CD70.

In some embodiments, CD73 is a targeted cell surface antigen and a binding moiety comprises the antigen binding domain of an anti-CD73 antibody. Accordingly, in some such embodiments, the antigen binding domain is derived from oleclumab, uliledlimab, mupadolimab, AK119, IB1325, BMS-986179, NZV930, JAB-BX102, Sym024, TB19, TB38, HBM1007, 3F7, mAb19, Hu001-MMAE, IPH5301, or INCA00186, as well as those disclosed in U.S. Pat. Nos. 9,938,356, 10,584,169, WO2022083723, WO2022037531, WO2021213466, WO2022083049, U.S. Pat. No. 10,822,426, WO2021259199, U.S. Pat. Nos. 10,100,129, 11,312,783, 11,174,319, 11,634,500, WO2021138467, WO2017118613, U.S. Pat. No. 9,388,249, WO2020216697, U.S. Ser. No. 11/180,554, U.S. Pat. No. 11,530,273, WO2019173692, WO2019170131, U.S. Pat. No. 11,312,785, WO2020098599, WO2020143836, WO2020143710, U.S. Pat. Nos. 11,034,771, 11,299,550, WO2020253568, WO2021017892, WO2021032173, WO2021032173, WO2021097223, WO2021205383, WO2021227307, WO2021241729, WO2022096020, WO2022105881, WO2022179039, WO2022214677, or WO2022242758, each of which is incorporated by reference for all that they teach about anti-CD73 antibodies and their properties, or an antigen-binding portion thereof. Each of these constitutes a means for binding CD73.

In some embodiments, CD90 is a targeted cell surface antigen and a binding moiety comprises the antigen binding domain of an anti-CD90 antibody. Accordingly, in some such embodiments, the antigen binding domain is derived from REA897, OX7, 5E10, K117, L127, or an antigen-binding portion thereof. Each of these constitutes a means for binding CD90.

In some embodiments, CD105 is a targeted cell surface antigen and a binding moiety comprises the antigen binding domain of an anti-CD105 antibody. Accordingly, in some such embodiments, the antigen binding domain is derived from carotuximab, TRC205, or huRH105, as well as those disclosed in U.S. Pat. Nos. 8,221,753, 9,926,375, WO2010039873, WO2010032059, WO2012149412, WO2015118031, WO2021118955, US20220233591, or US20230075244, each of which is incorporated by reference for all that they teach about anti-CD105 antibodies and their properties, or an antigen-binding portion thereof. Each of these constitutes a means for binding CD105.

In some embodiments, CD117 is a targeted cell surface antigen and a binding moiety comprises the antigen binding domain of an anti-CD117 antibody. Accordingly, in some such embodiments, the antigen binding domain is derived from briquilimab, barzolvolimab, CDX-0158, LOP628, MGTA-117, NN2101, CK6, JSP191, Ab85, 104D2, or SR1, as well as those disclosed in U.S. Pat. No. 7,915,391, WO2022159737, U.S. Pat. No. 9,540,443, WO2015050959, U.S. Pat. Nos. 9,789,203, 8,552,157, 10,406,179, 9,932,410, WO2019084067, WO2020219770, U.S. Pat. No. 10,611,838, WO2020076105, WO2021107566, U.S. Pat. No. 11,208,482, WO2021044008, WO2021099418, WO2022187050, or WO2023026791, WO2021188590, each of which is incorporated by reference for all that they teach about anti-CD117 antibodies and their properties, or an antigen-binding portion thereof. Each of these constitutes a means for binding CD117.

In some embodiments, CD133 is a targeted cell surface antigen and a binding moiety comprises the antigen binding domain of an anti-CD133 antibody. Accordingly, in some such embodiments, the antigen binding domain is derived from AC133, 293C3, CMab-43, or RW03, as well as those disclosed in WO2018045880, U.S. Pat. Nos. 8,722,858, 9,249,225, WO2014128185, U.S. Pat. Nos. 10,711,068, 10,106,623, WO2018072025, or WO2022022718, each of which is incorporated by reference for all that they teach about anti-CD133 antibodies and their properties, or an antigen-binding portion thereof. Each of these constitutes a means for binding CD133.

In some embodiments, CD137 is a targeted cell surface antigen and a binding moiety comprises the antigen binding domain of an anti-CD137 antibody. CD137 is also known as 4-1BB. Accordingly, in some such embodiments, the antigen binding domain is derived from YH004, urelumab (BMS-663513), utomilumab (PF-05082566), ADG106, LVGN6051, PRS-343, as well as those disclosed in WO2005035584, WO2012032433, WO2017123650, U.S. Pat. Nos. 11,203,643, 11,242,395, 11,555,077, US20230067770, U.S. Pat. Nos. 11,535,678, 11,440,966, WO2019092451, U.S. Pat. Nos. 10,174,122, 11,242,385, 10,716,851, WO2020011966, WO2020011964, or U.S. Pat. No. 11,447,558, each of which is incorporated by reference for all that they teach about CD137 antibodies and their properties, or an antigen-binding portion thereof. Each of these constitutes a means for binding CD137.

In some embodiments, CD146 is a targeted cell surface antigen and a binding moiety comprises the antigen binding domain of an anti-CD146 antibody. Accordingly, in some such embodiments, the antigen binding domain is derived from imaprelimab, ABX-MA1, huAA98, M2H, or IM1-24-3, as well as those disclosed in U.S. Pat. Nos. 10,407,506, 10,414,825, 6,924,360, 9,447,190, WO2014000338, U.S. Pat. No. 9,782,500, WO2018220467, U.S. Pat. No. 11,427,648, WO2019133639, WO2019137309, WO2020132190, or WO2022082073, each of which is incorporated by reference for all that they teach about CD146 antibodies and their properties, or an antigen-binding portion thereof. Each of these constitutes a means for binding CD146.

In some embodiments, CD166 is a targeted cell surface antigen and a binding moiety comprises the antigen binding domain of an anti-CD166 antibody. Accordingly, in some such embodiments, the antigen binding domain is derived from praluzatamab, AZN-L50, REA442, or AT002, as well as those disclosed in U.S. Pat. Nos. 10,745,481, 11,220,544, or WO2008117049, each of which is incorporated by reference for all that they teach about CD166 antibodies and their properties, or an antigen-binding portion thereof. Each of these constitutes a means for binding CD166.

In some embodiments, CD200 is a targeted cell surface antigen and a binding moiety comprises the antigen binding domain of an anti-CD200 antibody. Accordingly, in some such embodiments, the antigen binding domain is derived from samalizumab, OX-104, REA1067, B7V3V2, HPAB-0260-YJ, or TTI-CD200, as well as those disclosed in WO2007084321 or WO2019126536, each of which is incorporated by reference for all that they teach about CD200 antibodies and their properties, or an antigen-binding portion thereof. Each of these constitutes a means for binding CD200.

In some embodiments, CD205 is a targeted cell surface antigen and a binding moiety comprises the antigen binding domain of an anti-CD205 antibody. CD205 is also known as DEC205. Accordingly, in some such embodiments, the antibody comprises 3G9-2D2 (a component of CDX-1401) or LY75_A1 (a component of MEN1309) as well as those disclosed in U.S. Pat. Nos. 8,236,318, 10,081,682, or U.S. Pat. No. 11,365,258, each of which is incorporated by reference for all that they teach about anti-CD205 antibodies and their properties, or an antigen-binding portion thereof. Each of these constitutes a means for binding CD205.

In some embodiments, CD271 is a targeted cell surface antigen and a binding moiety comprises the antigen binding domain of an anti-CD271 antibody. Accordingly, in some such embodiments, the antigen binding domain is derived from REA844 or REAL709 as well as those disclosed in WO2022166802 which is incorporated by reference for all that it teaches about anti-CD271 antibodies and their properties, or an antigen-binding portion thereof. Each of these constitutes a means for binding CD271.

In some embodiments, BMPR2 is a targeted cell surface antigen and a binding moiety comprises the antigen binding domain of an anti-BMPR2 antibody. Accordingly, in some such embodiments, the antigen binding domain is derived from TAB-071CL (Creative Biolabs catalog no.) as well as those disclosed in U.S. Pat. No. 11,292,846 or WO2021174198, each of which is incorporated by reference for all that they teach about anti-BMPR2 antibodies and their properties, or an antigen-binding portion thereof. Each of these constitutes a means for binding BMPR2.

In some embodiments, claudin 18.2 (CLDN 18.2) is a targeted cell surface antigen and a binding moiety comprises the antigen binding domain of an anti-claudin 18.2 antibody. Accordingly, in some such embodiments, the antigen binding domain is derived from zolbetuximab, osemitamab, RC118, IBI-343, AZD0901, M108, SYSA1801, TORL-2-307-ADC, LM-302, ASKB589, gresonitamab, SPX-101, SKB315, Q-1802, GIVASTOMIG, LCAR-C18S, SOT102, CT041 as well as those disclosed in WO2013167259, WO2021032157, WO2021254481, WO2022007808, WO2021008463, WO2022111616, WO2018006882, WO2020147321, WO2019219089, US20200040101, WO2020025792, WO2020139956, WO2020135201, US20240228610, WO2021218874, WO2021027850, WO2021129765, WO2022068854, WO2021111003, each of which is incorporated by reference for all that it teaches about anti-claudin 18.2 antibodies and their properties, or an antigen-binding portion thereof. Each of these constitutes a means for binding claudin 18.2.

In some embodiments, CTLA-4 is a targeted cell surface antigen and a binding moiety comprises the antigen binding domain of an anti-CTLA-4 antibody. Accordingly, in some such embodiments, the antigen binding domain is derived from botensilimab, ipilimumab, nurulimab, quavonlimab, tremelimumab, zalifrelimab, ADG116, ADG126, ADU-1604, AGEN1181, BCD-145, BMS-986218, BMS-986249, BT-007, CS1002, GIGA-564, HBM4003, IB1310 JK08, JMW-3B3, JS007, KD6001, KN044, ONC-392, REGN4659, TG6050, XTX101, YH001, or an antigen-binding portion thereof. Each of these constitutes a means for binding CTLA-4.

In some embodiments, GD2 is a targeted cell surface antigen and a binding moiety comprises the antigen binding domain of an anti-GD2 antibody. Accordingly, in some such embodiments, the antigen binding domain is derived from dinutuximab, ganglidiximab, naxitamab, nivatrotamab, EMD 273063, hu14.18k322A, MORAb-028, 3F8BiAb, BCD-245, KM666, ATL301, Ektomab, as well as those disclosed in U.S. Pat. Nos. 9,777,068, 9,315,585, WO2004055056, U.S. Pat. Nos. 9,617,349, 9,493,740, US20210002384, U.S. Pat. No. 8,507,657, WO2001023573, WO2012071216, WO2018010846, U.S. Pat. No. 8,951,524, WO2023280880, U.S. Pat. No. 9,856,324, WO2015132604, WO2017055385, WO2019059771, WO2020020194, or an antigen-binding portion thereof. Each of these constitutes a means for binding GD2.

In some embodiments, GITR is a targeted cell surface antigen and a binding moiety comprises the antigen binding domain of an anti-GITR antibody. Accordingly, in some such embodiments, the antigen binding domain is derived from ragifilimab, TRX518, MK-4166, AMG 228, MED11873, BMS-986156, REGN6569, ASP1951, MK-1248, FRA154, GWN323, JNJ-64164711, ATOR-1144, or an antigen-binding portion thereof. Each of these constitutes a means for binding GITR.

In some embodiments, a low affinity IL-2 receptor is a targeted cell surface antigen (CD122 and/or CD132) and a binding moiety comprises the antigen binding domain of an anti-IL-2 receptor antibody. Accordingly, in some such embodiments, the antiCD122 antibody comprises ANV419, FB102, MiK-Beta-1 and the anti CD122 antibodies disclosed in WO2011127324, WO2017021540, WO2022212848, WO2022221409, WO2023078113, US20230272090, WO2024073723, or an antigen-binding portion thereof. Accordingly, in some such embodiments, the anti-CD132 antibody comprises REGN7257 and the anti-CD132 antibodies disclosed in WO2020160242, WO2017021540, WO2022212848, WO2023078113, US20230272089, or an antigen-binding portion thereof. Each of these constitutes a means for binding the low affinity IL-2 receptor (CD122 or CD132, as appropriate),

In some embodiments, a high affinity IL-2 receptor is a targeted cell surface antigen (CD25) and a binding moiety comprises the antigen binding domain of an anti-IL-2 receptor antibody. Accordingly, in some such embodiments, the antigen binding domain is derived from daclizumab, basiliximab, camidanlumab, vopitug, inolimomab, HuMAx-TAC, Xenopax, STI-003, RA8, RTX-003, and the anti-CD25 antibodies disclosed in WO2023031403, WO2006108670, WO2019175223, WO2019175215, WO2019175226, WO2004045512, WO2022104009, WO2020102591, or an antigen-binding portion thereof. Each of these constitutes a means for binding the high affinity IL-2 receptor (CD25).

In some embodiments, IL-7 receptor (CD127) is a targeted cell surface antigen and a binding moiety comprises the antigen binding domain of an anti-IL-7 receptor antibody. Accordingly, in some such embodiments, the antigen binding domain is derived from PF-06342674, GSK2618960, OSE-127, lusvertikimab, bempikibart, and the anti-CD127 antibodies disclosed in WO2011104687, WO2011094259, WO2013056984, WO2015189302, WO2017062748, WO2020154293, WO2020254827, WO2021222227, WO2023201316, or an antigen-binding portion thereof. Each of these constitutes a means for binding the CD127.

In some embodiments, IL-12 receptor is a targeted cell surface antigen and a binding moiety comprises the antigen binding domain of an anti-IL-12 receptor antibody. Accordingly, in some such embodiments, the antigen binding domain is derived from CBYY-10413, REA333, or an antigen-binding portion thereof. Each of these constitutes a means for binding the IL-12 receptor.

In some embodiments, IL-15 receptor a is a targeted cell surface antigen and a binding moiety comprises the antigen binding domain of an anti-IL-15 receptor a antibody. Accordingly, in some such embodiments, the antigen binding domain is derived from MAB1472-100, MAB5511, JM7A4, 5E3E1, JM7A4, 2639B, or an antigen-binding portion thereof. Each of these constitutes a means for binding the IL-15 receptor a.

In some embodiments, IL-18 receptor a is a targeted cell surface antigen and a binding moiety comprises the antigen binding domain of an anti-IL-18 receptor a antibody. Accordingly, in some such embodiments, the antigen binding domain is derived from H44, or an antigen-binding portion thereof. Each of these constitutes a means for binding the IL-18 receptor a.

In some embodiments, IL-21 receptor is a targeted cell surface antigen and a binding moiety comprises the antigen binding domain of an anti-IL-21 receptor antibody. Accordingly, in some such embodiments, the antigen binding domain is derived from 1D1C2, 19F5, 18A5, REA233, or an antigen-binding portion thereof. Each of these constitutes a means for binding the IL-21 receptor a.

In some embodiments, LAG-3 is a targeted cell surface antigen and a binding moiety comprises the antigen binding domain of an anti-LAG-3 antibody. Accordingly, in some such embodiments, the antigen binding domain is derived from relatlimab, tebotelimab, favezelimab, fianlimab, miptenalimab, HLX26, ieramilimab, GSK2831781, INCAGN2385, R07247669, encelimab, FS118, SHR-1802, Sym022, IB1110, IB1323, bavunalimab, EMB-02, ABL501, INCA32459, AK129, or an antigen-binding portion thereof. Each of these constitutes a means for binding LAG-3.

In some embodiments, MSCA-1 is a targeted cell surface antigen and a binding moiety comprises the antigen binding domain of an anti-MSCA-1 antibody. Accordingly, in some such embodiments, the antigen binding domain is derived from REAL219, W8B2, X9C3, or an antigen-binding portion thereof. Each of these constitutes a means for binding MSCA-1.

In some embodiments, OX40 is a targeted cell surface antigen and a binding moiety comprises the antigen binding domain of an anti-OX40 antibody. Accordingly, in some such embodiments, the antigen binding domain is derived from MED16469, ivuxolimab, rocatinlimab, GSK3174998, BMS-986178, vonlerizumab, INCAGN1949, tavolimab, BGB-A445, INBRX-106, BAT6026, telazorlimab, ATOR-1015, MED16383, cudarolimab, FS120, HFB301001, EMB-09, HLX51, Hu222, ABM193, or an antigen-binding portion thereof. Each of these constitutes a means for binding OX40.

In some embodiments, PD-1 is a targeted cell surface antigen and a binding moiety comprises the antigen binding domain of an anti-PD-1 antibody. Accordingly, in some such embodiments, the antigen binding domain is derived from nivolumab, pembrolizumab, camrelizumab, torpalimab, sintilimab, tislelizumab, cemiplimab, spartalizumab, serplulimab, cadonilimab, penpulimab, dostarlimab, zimberelimab, retifanlimab, pucotenlimab, pidilizumab, pidilizumab, balstilimab, ezabenlimab, AK112, geptanolimab, cetrelimab, prolgolimab, tebotelimab, sasanlimab, SG001, vudalimab, MED15752, rulonilimab, peresolimab, IB1318, budigalimab, MED10680, pimivalimab, QL1706, AMG 404, R07121661, lorigerlimab, nofazinlimab, sindelizumab, or an antigen-binding portion thereof. Each of these constitutes a means for binding PD-1.

In some embodiments, PODXL is a targeted cell surface antigen and a binding moiety comprises the antigen binding domain of an anti-PODXL antibody. Accordingly, in some such embodiments, the antigen binding domain is derived from MA11738, HPAB-3334LY, HPAB-MO612-YC, REA246, REA157, or an antigen-binding portion thereof. Each of these constitutes a means for binding PODXL.

In some embodiments, Sca-1 is a targeted cell surface antigen and a binding moiety comprises the antigen binding domain of an anti-Sca-1 antibody. Accordingly, in some such embodiments, the antigen binding domain is derived from CPP32 4-1-18, 2D4-C9-F1, AMM22070N, or an antigen-binding portion thereof. Each of these constitutes a means for binding SCA-1.

In some embodiments, SSEA-3 is a targeted cell surface antigen and a binding moiety comprises the antigen binding domain of an anti-SSEA-3 antibody. Accordingly, in some such embodiments, the antigen binding domain is derived from MC631, 2A9, 8A7, ND-742, 3H420, as well as those disclosed in U.S. Pat. No. 11,643,456 or WO2021138378, each of which is incorporated by reference for all that they teach about anti-SSEA-3 antibodies and their properties, or an antigen-binding portion thereof. Each of these constitutes a means for binding SSEA-3.

In some embodiments, SSEA-4 is a targeted cell surface antigen and a binding moiety comprises the antigen binding domain of an anti-SSEA-4 antibody. Accordingly, in some such embodiments, the antigen binding domain is derived from ch28/11, REA101, MC-813-70, ND-942-80, as well as those disclosed in U.S. Pat. Nos. 11,446,379, 10,273,295, 11,643,456, WO2019190952, or WO2021044039, each of which is incorporated by reference for all that they teach about anti-SSEA-4 antibodies and their properties, or an antigen-binding portion thereof. Each of these constitutes a means for binding SSEA-4.

In some embodiments, Stro-1 is a targeted cell surface antigen and a binding moiety comprises the antigen binding domain of an anti-Stro-1 antibody. Accordingly, in some such embodiments, the antigen binding domain is derived from STRO-1, TUSP-2, as well as those disclosed in US20130122022, which is incorporated by reference for all that it teaches about anti-Stro-1 antibodies and their properties, or an antigen-binding portion thereof. Each of these constitutes a means for binding Stro-1.

In some embodiments, Stro-4 is a targeted cell surface antigen and a binding moiety comprises the antigen binding domain of an anti-Stro-4 antibody. Accordingly, in some such embodiments, the antigen binding domain is derived from STRO-4, efungumab, 4C5, as well as those disclosed in U.S. Pat. No. 7,722,869, US20110280881, U.S. Pat. Nos. 9,115,192, 10,273,294, 10,457,726, WO2023091148, each of which is incorporated by reference for all that they teach about anti-Stro-4 antibodies and their properties, or an antigen-binding portion thereof. Each of these constitutes a means for binding Stro-4 (also known as heat shock protein-90).

In some embodiments, SUSD2 is a targeted cell surface antigen and a binding moiety comprises the antigen binding domain of an anti-SUSD2 antibody. Accordingly, in some such embodiments, the antigen binding domain is derived from REA795, CBXS-3571, CBXS-1650, CBXS-1989, CBXS-1671, CBXS1990, CBXS-3676, 1279B, EPR8913(2), W5C5, or an antigen-binding portion thereof. Each of these constitutes a means for binding SUSD2.

In some embodiments, TIM-3 is a targeted cell surface antigen and a binding moiety comprises the antigen binding domain of an anti-TIM-3 antibody. Accordingly, in some such embodiments, the antigen binding domain is derived from TQB2618, sabatolimab, cobolimab, R07121661, INCAGN02390, AZD7789, surzebiclimab, LY3321367, Sym023, BMS-986258, SHR-1702, LY3415244, LB1410, or an antigen-binding portion thereof. Each of these constitutes a means for binding TIM-3.

In some embodiments, TREM2 is a targeted cell surface antigen and a binding moiety comprises the antigen binding domain of an anti-TREM2 antibody. Accordingly, in some such embodiments, the antigen binding domain is derived from P137012 as well as those disclosed in U.S. Pat. Nos. 10,508,148, 10,676,525, WO2017058866, U.S. Pat. Nos. 11,186,636, 11,124,567, WO2020055975, U.S. Pat. No. 11,492,402, WO2020121195, WO2023012802, WO2021101823, WO2023047100, WO2022032293, WO2022241082, WO2023039450, or WO2023039612, each of which is incorporated by reference for all that they teach about anti-TREM2 antibodies and their properties, or an antigen-binding portion thereof. Each of these constitutes a means for binding TREM2.

In some embodiments, G protein-coupled receptor, class C, group 5, member D (GPRC5D) is a targeted cell surface antigen and a binding moiety comprises the antigen binding domain of an anti-GPRC5D antibody. Accordingly, in some such embodiments, the antigen binding domain of an anti-GPRC5D antibody is derived from talquetamab, forimtamig, BMS-986393, IBI-3003, QLS32015, SIM0500, or EPR28376-41, or is disclosed in WO2018017786, WO2016090329, WO2022174813, WO2023236889, WO2018147245, WO2024079015, WO2019154890, WO2021018859, WO2021018925, WO2020092854, WO2024031091, WO2020148677, WO2022175255, WO2022222910, WO2022247804, WO2022247756, WO2023078382, WO2023125728, WO2023143537, WO2024046239, or WO2024131962 each of which is incorporated by reference for all that they teach about anti-GPRC5D antibodies and their properties, or an antigen-binding portion thereof. Each of these constitutes a means for binding GPRC5D.

In some embodiments, FCRL5 (CD307E) is a targeted cell surface antigen and binding moiety comprises the antigen binding domain of an anti-FCRL5 antibody. Accordingly, in some such embodiments, the antigen binding domain of an anti-FCRL5 antibody is derived from cevostamab, 2A10H7, 307307, 2A10D6, EPR27365-87, EPR26948-19, or EPR26948-67, or is disclosed in WO2016090337, WO2017096120, WO2022263855, or WO2024047558 each of which is incorporated by reference for all that they teach about anti-FCRL5 antibodies and their properties, or an antigen-binding portion thereof. Each of these constitutes a means for binding FCRL5.

In some embodiments, LRRC15 is a targeted cell surface antigen and binding moiety comprises the antigen binding domain of an anti-LRRC15 antibody. Accordingly, in some such embodiments, the antigen binding domain of an anti-LRRC15 antibody is derived from samrotamab or DUNP19 or is disclosed in WO2005037999, WO2021022304, WO2024081729, WO2021102332, WO2021202642, WO2022157094, or WO2024158047, each of which is incorporated by reference for all that they teach about anti-LRRC15 antibodies and their properties, or an antigen-binding portion thereof. Each of these constitutes a means for binding LRRC15.

In still further embodiments, a tLNP is targeted to a tumor cell. In some embodiments, the tumor cell expresses one of the antigens described above and the tLNP is targeted to antigen expressing tumors using the same means as described above. In other embodiments the tLNP is targeted to some other tumor antigen, such as those enumerated in U.S. Provisional Application No. 63/371,742, filed on Aug. 17, 2022, entitled CONDITIONING FOR IN VIVO IMMUNE CELL ENGINEERING which is incorporated by reference for all that it teaches about the delivery of nucleic acids into tumor cells using tLNP that is not inconsistent with the present disclosure.

### Nucleic Acid

In certain embodiments, the disclosed LNP and tLNP comprise a payload comprising or consisting of one or more nucleic acid species. In some embodiments, the LNP or tLNP payload comprises only one nucleic acid species while in other embodiments the LNP or tLNP payload comprises multiple nucleic acid species, for example, 2, 3, or 4 nucleic acid species. For example, in embodiments in which the payload comprises a nucleic acid encoding a CAR or immune cell engager (ICE), the payload can comprise or consist of 1) a single nucleic acid species encoding a single species of CAR or ICE, 2) a single nucleic acid species encoding 2 or more species of CAR or ICE (or a mixture of CAR and ICE) such as a bicistronic or multicistronic mRNA in which each CAR and/or ICE has specificity for a same target antigen, 3) a single nucleic acid species encoding 2 or more species of CAR or ICE (or a mixture of CAR and ICE) such as a bicistronic or multicistronic mRNA in which at least one CAR and/or ICE has specificity for a different target antigen than the other(s), 4) two or more nucleic acid species encoding 2 or more species of CAR or ICE (or a mixture of CAR and ICE) in which each CAR and/or ICE has specificity for a same target antigen, 5) two or more nucleic acid species encoding 2 or more species of CAR or ICE (or a mixture of CAR and ICE) in which at least one CAR and/or ICE has specificity for a different target antigen than the other(s). When two or more CAR and/or ICE have specificity for a same target antigen, they can have specificity for same or different epitopes of the same target antigen. Further variations will be apparent to one of skill in the art (e.g., multiple bi- or multicistronic nucleic acids, nucleic acids encoding a TCR and the like). The nucleic acid can be RNA or DNA. The nucleic acid can be multicistronic, for example, bicistronic.

In some embodiments, LNPs or tLNPs of this disclosure further comprise a nucleic acid payload. In various embodiments, a nucleic acid is an mRNA, a self-replicating RNA, a circular RNA, a siRNA, a miRNA, DNA, a gene editing component (for example, a guide RNA, a tracr RNA, an sgRNA), a gene writing component, an mRNA encoding a gene or base editing protein, a zinc-finger nuclease, a TALEN, a CRISPR nuclease, such as Cas9, a DNA molecule to be inserted or serve as a template for repair), and the like, or a combination thereof. In some embodiments, the nucleic acid comprises small interfering RNA (siRNA), microRNA (miRNA), antisense oligonucleotide (ASO). In some embodiments, the nucleic acid comprises a self-replicating RNA or a circular RNA. In some embodiments, the mRNA encodes a reprogramming agent or comprises or encodes a conditioning agent. In some embodiments, the mRNA (linear, circular, or self-replicating) comprises an miRNA binding site. In some embodiments, an mRNA encodes a chimeric antigen receptor (CAR). In other embodiments, an mRNA encodes a gene-editing or base-editing or gene writing protein. In some embodiments, a nucleic acid is a guide RNA. In some embodiments, an LNP or tLNP comprises both a gene- or base-editing or gene writing protein-encoding mRNA and one or more guide RNAs. CRISPR nucleases can have altered activity, for example, modifying the nuclease so that it is a nickase instead of making double-strand cuts or so that it binds the sequence specified by the guide RNA but has no enzymatic activity. Base-editing proteins are often fusion proteins comprising a deaminase domain and a sequence-specific DNA binding domain (such as an inactive CRISPR nuclease).

In some embodiments, the reprogramming agent comprises an immune receptor (for example, a chimeric antigen receptor or a T cell receptor) or an immune cell engager (for example, a bispecific T cell engager (BiTE), a bispecific killer cell engager (BiKE), a trispecific kill cell engager (TriKE), a dual affinity retargeting antibody (DART), a TRIDENT (linking two DART units or a DART unit and a Fab domain), a macrophage engager (e.g., BiME), an innate cell engager, and the like).

In some embodiments, the nucleic acid is an RNA, for example, mRNA, and the RNA comprises at least one modified nucleoside. In some embodiments, the modified nucleoside is pseudouridine, Ni-methylpseudouridine, 5-methylcytosine, 5-methyluridine, N-methyladenosine, 2-O-methyluridine, or 2-thiouridine. In certain embodiments, all of the uridines are substituted with a modified nucleoside. Further disclosure of modified nucleosides and their use can be found in U.S. Pat. No. 8,278,036 which is incorporated herein by reference for those teachings.

In some embodiments, the reprogramming agent encodes or is a gene/genome editing component. In some embodiments, the gene/genome editing component is a guide RNA for an RNA-directed nuclease or other nucleic acid editing enzyme, clustered regularly interspaced short palindromic repeat RNA (crisprRNA), a trans-activating clustered regularly interspaced short palindromic repeat RNA (tracrRNA). In some embodiments, the gene/genome editing component is a nucleic acid-encoded enzyme, such as RNA-guided nuclease, a gene or base editing protein, a zinc finger nuclease (ZFN), a transcription activator-like effector nuclease (TALEN), a meganuclease, a transposase, or a CRISPR nuclease (e.g., Cas9 or Cas 12, etc.). In some embodiments, the gene/genome editing component is DNA to be inserted or that serves as a template in gene or genome editing for example a template for repair of a double-strand break.

In some embodiments comprising multiple agents, the nucleic acid can be multicistronic. In other embodiments comprising multiple agents or components, each agent or component is encoded or contained is a separate nucleic acid species. In some embodiments involving multiple payload nucleic acid species, two or more nucleic acid species are packaged together in a single LNP species. In other embodiments, a subset of the payload nucleic acid species to be delivered, (e.g., a single nucleic acid species) is packaged in one LNP or tLNP species while another subset of the nucleic acid species is packaged in another LNP or tLNP species. The different (t)LNP species can differ by only the payload they contain. The different (t)LNP species can be combined in a single formulation or pharmaceutical composition for administration.

Methods of Making an LNP or tLNP

In some aspects, the present disclosure provides a method of making a LNP or tLNP comprising mixing of an aqueous solution of a nucleic acid (or other negatively charged payload) and an alcoholic solution of the lipids in proportions disclosed herein. In particular embodiments, the mixing is rapid.

The aqueous solution is buffered at pH of about 3 to about 5, for example, without limitation, with citrate or acetate. In various embodiments, the alcohol can be ethanol, isopropanol, t-butanol, or a combination thereof. In some embodiments, rapid mixing is accomplished by pumping the two solutions through a T-junction or with an impinging jet mixer. Microfluidic mixing through a staggered herringbone mixer (SHM) or a hydrodynamic mixer (microfluidic hydrodynamic focusing), microfluidic bifurcating mixers, and microfluidic baffle mixers can also be used. After the LNPs are formed they are diluted with buffer, for example phosphate, HEPES, or Tris, in a pH range of 6 to 8.5 to reduce the alcohol (ethanol) concentration. The diluted LNPs are purified either by dialysis or ultrafiltration or diafiltration using tangential flow filtration (TFF) against a buffer in a pH range of 6 to 8.5 (for example, phosphate, HEPES, or Tris) to remove the alcohol. Alternatively, one can use size exclusion chromatography. Once the alcohol is completely removed the buffer is exchanged with like buffer containing a cryoprotectant (for example, glycerol or a sugar such as sucrose, trehalose, or mannose). The LNPs are concentrated to a desired concentrated, followed by 0.2 μm filtration through, for example, a polyethersulfone (PES) or modified PES filter and filled into glass vials, stoppered, capped, and stored frozen. In alternative embodiments, a lyoprotectant is used and the LNP lyophilized for storage instead of as a frozen liquid. Further methodologies for making LNP can be found, for example, in U.S. Patent Application Publication Nos. US2020/0297634, US2013/0115274, and International Patent Application Publication No. WO2017/048770, each of which is incorporated by reference for all that they teach about the production of LNP.

Some aspects are a method of making a tLNP comprising rapid mixing of an aqueous solution of a nucleic acid (or other negatively charged payload) and an alcoholic solution of the lipids as disclosed for LNP. In some embodiments, the lipid mixture includes functionalized PEG-lipid, for later conjugation to a targeting moiety. As used herein, functionalized PEG-lipid refers to a PEG-lipid in which the PEG moiety has been derivatized with a chemically reactive group (such as, maleimide, N-hydroxysuccinimide (NHS) ester, Cys, azide, alkyne, and the like) that can be used for conjugating a targeting moiety to the PEG-lipid, and thus, to the LNP comprising the PEG-lipid. In other embodiments, the functionalized PEG-lipid is inserted into an LNP subsequent to initial formation of an LNP from other components. In either type of embodiment, the targeting moiety is conjugated to functionalized PEG-lipid after the functionalized PEG-lipid containing LNP is formed. Protocols for conjugation can be found, for example, in Parhiz et al., 2018, J. Controlled Release 291:106-115 and Tombacz et al., 2021, Molecular Therapy 29(11):3293-3304, each of which is incorporated by reference for all that it teaches about conjugation of PEG-lipids to binding moieties. Alternatively, the targeting moiety can be conjugated to the PEG-lipid prior to insertion into pre-formed LNP.

In certain embodiments of the preparation methods of tLNP, the method comprises:
i). forming an initial LNP by mixing all components of the tLNP, in proportions disclosed herein, except for the one or more functionalized PEG-lipids and the one or more targeting moieties;
ii). forming a pre-conjugation tLNP by mixing the initial LNP with the one or more functionalized PEG-lipids; and
iii). forming the tLNP by conjugating the pre-conjugation tLNP with the one or more targeting moieties.

In certain embodiments of the preparation methods of tLNP, the method comprises:
i). forming a pre-conjugation tLNP by mixing all components of the tLNP, in proportions disclosed herein, including the one or more functionalized PEG-lipids, except for the one or more targeting moieties; and
ii). forming the tLNP by conjugating the pre-conjugation tLNP with the one or more targeting moieties.

In certain embodiments of the preparation methods of tLNP, the method comprises:
i). forming one or more conjugated functionalized PEG-lipids by conjugating the one or more functionalized PEG-lipids with the one or more targeting moieties; and
ii) forming the tLNP by mixing all components of the tLNP, in proportions disclosed herein, including the one or more conjugated functionalized PEG-lipids.

In certain embodiments of the preparation methods of tLNP, the method comprises:
i). forming one or more conjugated functionalized PEG-lipids by conjugating the one or more functionalized PEG-lipids with the one or more targeting moieties;
ii) forming an LNP by mixing all components of the tLNP, except the one or more conjugated functionalized PEG-lipids; and
iii) forming the tLNP by mixing the initial LNP with the one or more conjugated functionalized PEG-lipids.

After the conjugation the tLNPs are purified by dialysis, tangential flow filtration, or size exclusion chromatography, and stored, as disclosed above for LNPs.

The encapsulation efficiency of the nucleic acid by the LNP or tLNP is typically determined with a nucleic acid binding fluorescent dye added to intact and lysed aliquots of the final LNP or tLNP preparation to determine the amounts of unencapsulated and total nucleic acid, respectively. Encapsulation efficiency is typically expressed as a percentage and calculated as 100×(T−U)/T where T is the total amount of nucleic acid and U is the amount of unencapsulated nucleic acid. In various embodiments, the encapsulation efficiency is 280%, 285%, 290%, or 295%.

Methods of Delivering a Payload into a Cell

In other aspects, disclosed herein are methods of delivering a nucleic acid (or other negatively charged payload) into a cell comprising contacting the cell with LNP or tLNP as disclosed herein. Accordingly, each of the herein disclosed genera, subgenera, and or species of LNP or tLNP disclosed herein including those based on the inclusion or exclusion of particular lipids, particular lipid compositions, particular payloads, and/or particular targeting moieties can be used in defining the scope of the methods of delivering a payload to a cell. In some embodiments, the contacting takes place ex vivo. In some embodiments, the contacting takes place extracorporeally. In some embodiments, the contacting takes place in vivo. In some embodiments, an LNP or tLNP is contacted with target cells in vivo, by systemic or local administration. In some embodiments, the in vivo contacting comprises intravenous, intramuscular, subcutaneous, intralesional, intranodal or intralymphatic administration. In some embodiments, administration is by intravenous or subcutaneous infusion or injection. In some embodiments, administration is by intraperitoneal or intralesional infusion injection. In further instances, transfection of hepatocytes is reduced as compared to tLNPs comprising a conventional ionizable cationic lipid, such as ALC-0315. In some embodiments, an LNP or tLNP is administered 1-3 times a week for 1, 2, 3, or 4 weeks. In some embodiments, toxicity is confined (or largely confined) to grades of 0 or 1 or 2, as discussed above.

The herein disclosed LNP and tLNP compositions and formulations have reduced toxicity as compared to widely used prior art LNP compositions such as those containing ALC-0315. In various embodiments the toxicity can be described as an observable toxicity, a substantial toxicity, a severe toxicity, or an acceptable toxicity, or a dose-limiting toxicity (such as but not limited to a maximum tolerated dose (MTD)). By an observable toxicity it is meant that while a change is observed the effect is negligible or mild. By substantial toxicity it is meant that there is a negative impact on the patient's overall health or quality of life. In some instances, a substantial toxicity can be mitigated or resolved with other ongoing medical intervention. By a severe toxicity it is meant that the effect requires acute medical intervention and/or dose reduction or suspension of treatment. The acceptability of a toxicity will be influenced by the particular disease being treated and its severity and the availability of mitigating medical intervention. In some embodiments, toxicity is confined (or largely confined) to an observable toxicity. In some embodiments, toxicity is confined (or largely confined) to grades of 0 or 1 or 2.

In some embodiments, the payload is a nucleic acid and the method of delivering is a method of transfecting. In some embodiments, the nucleic acid payload comprises an mRNA, circular RNA, self-amplifying RNA, or guide RNA. Nucleic acid structures and especially mRNA structures, as well as individual RNA molecules encoding particular polypeptides, that are well-adapted to delivery by LNP or tLNP are disclosed in U.S. Provisional Patent Application No. 63/595,753 filed Nov. 2, 2023, U.S. Provisional Patent Application No. 63/611,092 filed Dec. 15, 2023, and U.S. Provisional Patent Application Number (Attorney Docket Number 23-1871-US-PRO3), filed May 31, 2024, each of which is incorporated by reference for all that it teaches about nucleic acid payloads for in vivo transfection and their design.

In some embodiments, the payload comprises a nucleic acid encoding an immune receptor or immune cell engager and the method of delivering is also a method of reprogramming an immune cell. In some embodiments, the payload comprises a nucleic acid that encodes, or is, a BRM and the method of delivering is also a method of providing a conditioning agent. In various embodiments, the BRM or conditioning agent is a gamma chain receptor cytokine such as IL-2, IL-7, IL-15, IL-15/15Ralpha, IL-21; an immune modulating cytokine such as IL-12, IL-18; a chemokine such as RANTES, IP10, MIG; or another BRM such as Flt3, GM-CSF, and G-CSF.

In some embodiments, the payload comprises a nucleic acid encoding a gene/genome editing enzyme and/or a guide RNA or other component of a gene/genome editing system and the method of delivering is also a method of reprogramming a cell. In some instances, the cell is an immune cell. In some instances, the cell is an HSC. In some instances, the cell is an MSC.

In certain embodiments comprising delivering the payload into an immune cell, the binding moiety binds to a lymphocyte surface molecule or a monocyte surface molecule. Lymphocyte surface molecules include CD2, CD3, CD4, CD5, CD7, CD8, CD28, 4-1BB (CD137), CD166, CTLA-4, OX40, PD-1, GITR, LAG-3, TIM-3, CD25, low affinity IL-2 receptor, IL-7 receptor, IL-12 receptor, IL-15 receptor, IL-18 receptor, and IL-21 receptor. Monocyte surface molecules include CD5, CD14, CD16a, CD32, CD40, CD11b (Mac-1), CD64, DEC205, CD68, and TREM2. Exemplary antibodies that can provide antigen binding domains to bind these surface molecules are disclosed herein. Such antibodies, individually and collectively, constitute means for binding to an immune cell (or leukocyte)—or to a lymphocyte or monocyte, as indicated.

In certain embodiments comprising delivering the payload into a stem cell, the binding moiety binds to a HSC surface molecule or a MSC surface molecule. HSC surface molecules include CD117, CD34, CD44, CD90 (Thy1), CD105, CD133, BMPR2, and Sca-1. MSC surface molecules include CD70, CD105, CD73, Stro-1, SSEA-4, CD271, CD146, GD2, SSEA-3, SUSD2, Stro-4, MSCA-1, CD56, CD200, PODXL, CD13, CD29, CD44, and CD10. Exemplary antibodies that can provide antigen binding domains to bind these surface molecules are disclosed herein above. Such antibodies, individually and collectively, constitute means for binding to a stem cell—or to an HSC or MSC, as indicated.

### Methods of Treatment

In certain aspects, this disclosure provides methods of treating a disease or disorder comprising administering a tLNP of this disclosure to a subject in need thereof. Each of the herein disclosed genera, subgenera, and or species of LNP or tLNP disclosed herein including those based on the inclusion or exclusion of particular lipids, particular lipid compositions, particular payloads, and/or particular targeting moieties can be used in defining the scope of the methods of treatment.

In some embodiments, a subject is a human. In some embodiments, a tLNP is administered systemically. In some embodiments, a tLNP is administered by intravenous or subcutaneous infusion or injection. In some embodiments, a tLNP is administered locally. In some embodiments, a tLNP is administered by intraperitoneal or intralesional infusion injection.

In further embodiments, a tLNP can be administered in combination with the standard of care for a particular indication, such as corticosteroids (e.g., prednisone) for management of myositis or lupus nephritis. In certain cases, myositis is also treated with methotrexate, which can be combined with immunosuppressive agents (e.g., azathioprine, mycophenolate mofetil, tacrolimus), which are usually required in addition to corticosteroids. For membranous nephropathy, cyclical steroids and cyclophosphamide might be used in combination with tLNPs of this disclosure. In other cases, an anti-IL-6, such as tocilizumab, can also be used as a pretreatment or in combination with tLNPs of this disclosure. These combinations can be administered concurrently or sequentially.

In some embodiments, the disease or disorder is an autoimmune disease. Examples of autoimmune disease include, without limitation, myocarditis, acute idiopathic thrombocytopenic purpura, chronic idiopathic thrombocytopenic purpura, dermatomyositis, Sydenham's chorea, myasthenia gravis, systemic lupus erythematosus, fibrosing alveolitis, multiple sclerosis, rheumatic fever, polyglandular syndromes, agranulocytosis, autoimmune hemolytic anemias, bullous pemphigoid, Wegener's granulomatosis, membranous nephropathy, amyotrophic lateral sclerosis, tabes dorsalis, giant cell arteritis/polymyalgia, pernicious anemia, rapidly progressive glomerulonephritis, IgA nephropathy, polyarteritis nodosa, ankylosing spondylitis, allergic responses, insulin-resistant diabetes, psoriasis, diabetes mellitus, Addison's disease, Grave's disease, diabetes, endometriosis, celiac disease, Crohn's disease, Henoch-Schonlein purpura, ulcerative colitis, Goodpasture's syndrome, thromboangitisubiterans, Sjögren's syndrome, aplastic anemia, rheumatoid arthritis, sarcoidosis, scleritis, a T cell-mediated autoimmunity or a B cell-mediated autoimmunity, a B cell-mediated (antibody-mediated) autoimmune disease, necrotizing myopathy, chronic inflammatory demyelinating polyneuropathy (CIDP), neuromyelitis optica (NMO) myositis, neuromyelitis optica spectrum disorders, pemphigus vulgaris, systemic sclerosis, antisynthetase syndrome (idiopathic inflammatory myopathy), lupus nephritis, membranous nephropathy, Fanconi anemia, and vasculitis.

In some embodiments, the autoimmune disease is a T cell-mediated autoimmunity or a B cell-mediated autoimmunity. In some instances, the B cell-mediated autoimmune disease is myositis (such as anti-synthetase myositis), lupus nephritis, membranous nephropathy, systemic lupus erythematosus, anti-neutrophilic cytoplasmic antibody (ANCA) vasculitis, neuromyelitis optica spectrum disorder (NMOSD), myasthenia gravis, pemphigus vulgaris, rheumatoid arthritis, dermatomyositis, immune mediated necrotizing myopathy (IMNM), anti-synthetase syndrome, polymyositis, systemic sclerosis, diffuse cutaneous systemic sclerosis, limited cutaneous systemic sclerosis, anti-synthetase syndrome (idiopathic inflammatory myopathy), stiff person syndrome, myeloid oligodendrocyte glycoprotein autoantibody associated disease (MOGAD), amyloid light-chain amyloidosis, multiple sclerosis, relapsing-remitting multiple sclerosis, secondary progressive multiple sclerosis, primary progressive multiple sclerosis, non-active secondary progressive multiple sclerosis, Sjörgen's syndrome, IgA nephropathy, IgG4-related disease, or Fanconi anemia. In certain embodiments, the B cell-mediated autoimmune disease is myositis, lupus nephritis, membranous neuropathy, scleroderma, systemic lupus erythematosus, myasthenia gravis, ANCA vasculitis, multiple sclerosis, or pemphigus vulgaris. In certain embodiments, the B cell-mediated autoimmune disease is myositis, lupus nephritis, membranous neuropathy, or scleroderma. In certain embodiments, the B cell-mediated autoimmune disease is myositis. In some instances, the myositis is anti-synthetase myositis. In certain embodiments, the B cell-mediated autoimmune disease is systemic lupus erythematosus, myasthenia gravis, ANCA vasculitis, multiple sclerosis, or pemphigus vulgaris.

In some embodiments, the disease or disorder is rejection of an allogeneic organ or tissue graft. Pre-existing antibodies and/or B cells, in their role as antigen presenting cells, can facilitate rapid immune rejection through known mechanisms hence depleting a large number of B cells can help prevent allograft rejection.

In some embodiments, the disease or disorder is a cancer. Examples of cancers include, without limitation, carcinomas, sarcomas, and hematologic cancers. In some embodiments, the hematologic cancer is a lymphoma, leukemia, or myeloma. In some instances, the hematologic cancer is a B lineage or T lineage cancer. In some instances, the B lineage cancer is multiple myeloma, diffuse large B cell lymphoma, acute myeloid leukemia, Mantie Cell lymphoma, follicular lymphoma, B acute lymphoblastic leukemia, chronic lymphocytic leukemia, or myelodysplastic syndrome. In some embodiments, the cancer is a sarcoma. In some embodiments, the cancer is a carcinoma, such as breast cancer, colon cancer, ovarian cancer, lung cancer, testicular cancer, or pancreatic cancer. In some embodiments, the cancer is melanoma.

In some embodiments, the disease or disorder is a genetic disease or disorder such as a monogenic genetic disease. In some instances, the genetic disease or disorder is a hemoglobinopathy, for example, sickle cell disease or s-thalassemia.

In some embodiments, the disease or disorder is a fibrotic disease or disorder. In some instances, the fibrotic disease is cardiac fibrosis, arthritis, idiopathic pulmonary fibrosis, and nonalcoholic steatohepatitis (also known as metabolic dysfunction-associated steatohepatitis). In other instances, the disorder involves tumor-associated fibroblasts.

In some embodiments, a tLNP of this disclosure comprises a nucleic acid encoding a chimeric antigen receptor (CAR). The receptors are chimeric because they combine both antigen-binding and T cell activating functions into a single receptor. In some embodiments, a nucleic acid encoding a CAR refers to one or more nucleic acid species encoding one or more CARs; for example, a single or multiple species of nucleic acid encoding a single CAR species, or multiple species of nucleic acid encoding multiple CAR species. In some instances, these multiple CAR species have a same specificity while in other instances they have multiple specificities. In some embodiments, a CAR of this disclosure is multispecific, for example, bispecific, comprising multiple antigen binding moieties each specific for separate antigens. For example, the CAR in LCAR-AIO targets three antigens—CD19, CD20 and CD22 (see, Blood (2021) 138 (Supplement 1): 1700). In some embodiments, a CAR can comprise an extracellular binding domain that specifically binds a target antigen, a transmembrane domain, and one or more intracellular signaling domains. In some embodiments, a CAR can further comprise one or more additional elements, including one or more signal peptides, one or more extracellular hinge domains, or one or more intracellular costimulatory domains. Domains can be directly adjacent to one another, or there can be one or more amino acids linking the domains. The signal peptide can be derived from an antibody, a TCR, CD8 or other type 1 membrane proteins, preferably a protein expressed in a T or other immune cell. The transmembrane domain can be one associated with any of the potential intracellular domains or from another type 1 membrane protein, such as TCR alpha, beta, or zeta chain, CD3 epsilon, CD4, CD8, or CD28, amongst other possibilities known in the art. The transmembrane domain can further comprise a hinge domain located between the extracellular binding domain and the hydrophobic membrane-spanning region of the transmembrane domain. In some but not all embodiments, the hinge domain and transmembrane domain are contiguous sequences in the same source protein. In some instances, the hinge and membrane-spanning domains are derived from CD28. In other instances, the hinge and membrane-spanning domains are derived from CD8α. The intracellular signaling domain can be derived from the CD3 zeta chain, DAP10, DAP12, FcγRII, FcsRI, or an immunoreceptor tyrosine-based activation motif (ITAM) bearing cytoplasmic domain, amongst other possibilities known in the art. The intracellular costimulatory domain can be derived from CD27, CD28, 4-1BB, OX40, or ICOS, amongst other possibilities known in the art.

In certain embodiments, CARs are used to treat a disease or condition associated with a target cell that expresses the antigen targeted by the CAR. For example, in some embodiments, an anti-CD19 or anti-CD20 CAR can be used to target and treat B cell malignancies or B cell-mediated autoimmune conditions or diseases (e.g., having an immune cell targeting moiety, such as an anti-CD8 antibody). In other embodiments, an anti-FAP CAR can be used to target and treat solid tumors or fibrosis (e.g., cardiac fibrosis, cancer-associated fibroblasts), which can also have an immune cell targeting moiety, such as an anti-CD8 antibody. Examples of CARs that can be used in accordance with the embodiments described herein include to those disclosed in U.S. Pat. Nos. 7,446,190, 9,328,156, 11,248,058, US20190321404, WO2019119822, WO2019159193, WO2020011706, WO2022125837, and WO02024086190 (anti-CD19), U.S. Pat. No. 10,287,35 (anti-CD19), U.S. Pat. No. 10,442,867 and US2021/0363245 (anti-CD19 and anti-CD20), U.S. Pat. No. 10,543,263 (anti-CD22), WO02016149578 (anti-CD19 and anti-CD22), U.S. Pat. Nos. 10,316,101, 11,623,961 WO2015052538, WO2016166630, WO2017130223, WO2017173256, WO2019085102, WO2019241426, WO2020065330, WO2020038146, WO2020190737, WO2021091945 (anti-BCMA), WO02016130598 (anti-BCMA and syndecan-1), U.S. Pat. No. 10,426,797 (anti-CD33), U.S. Pat. No. 10,844,128 (anti-CD123), U.S. Pat. Nos. 10,428,141, 10,752,684, 11,723,925, WO2016187216, WO2017156479, WO2018197675, WO2020014366, and WO2020198531 (anti-ROR1), WO2022247756, WO2020148677, WO2020092854, & US20230331872 (anti-GPRC5D), WO2016090337, WO2022263855, & WO2024047558 (anti-FCRL5), and US2021/0087295 (anti-FAP), each of which is incorporated by reference for all that it teaches about CAR structure and function generically and with respect to the CAR's antigenic specificity and target indications to the extent that it is not inconsistent with the present disclosure. Each CAR constitutes means for targeting an immune cell, for example, a T cell, to the indicated antigen.

Exemplary target antigens against which a CAR, TCR, or ICE can have specificity include, but are not limited to, B cell maturation agent (BCMA)^{†‡}, CA9^{†‡}, CD4^{†‡}, CD5^{†‡}, CD19*^{†‡}, CD20 (MS4A1)*^{†‡}, CD22*^{†‡}, FCRL5^{†‡}, GPRC5D^{†‡}, CD23*^{†‡}, CD30 (TNFRSF8)*^{†‡}, CD33*^{†‡}, CD38*^{†‡}, CD44*^{‡}, CD70*^{†‡}, CD133^{‡}, CD174, CD274 (PD-L1)*^{†‡}, CD276 (B7-H3)^{†‡}, CEACAM5*^{†‡}, CLL1^{‡}, CSPG4*^{‡}, EGFR*^{†‡}, EGFRvIII*, EPCAM*^{†‡}, EPHA2*^{‡}, ERBB2*^{‡}, FAP*^{†‡}, FOLH1, FOLR1*^{†‡}, GD2*^{†‡}, GPC3*^{†‡}, GPNMB*^{‡}, IL1RAP^{†‡}, IL3RA*^{‡}, IL13RA2*^{‡}, Kappa*, KDR (VEGFR2)*^{‡}, CD171 (L1CAM)*^{‡}, Lambda*, MET*^{‡}, MSLN (mesothelin)*^{†‡}, MUC1*^{†‡}, NCAM1 (CD56)*^{‡}, PD-1 (CD279)^{†‡}, PSCA^{‡}, ROR1^{†‡}, CD138 (SDC1)*^{‡}, CD319 (SLAMF7)*^{†‡}, CD248 (TEM1)^{‡}, ULBP1, ULBP2, and G-protein coupled receptor family C group 5 member D (GPRC5D)^{†‡} (associated with leukemias); CD319 (SLAMF7)*^{†‡}, CD38*^{†‡}, CD138^{†‡}, GPRC5D^{†‡}, CD267 (TACI)^{‡}, and BCMA^{†‡} (associated with myelomas); and GD2*^{†‡}, GPC3*^{†‡}, HER2*^{†‡}, EGFR*^{†‡}, EGFRvIII*, CD276 (B7H3)^{†‡}, PSMA*^{†‡}, PSCA^{‡}, CAIX (CA9)^{†‡}, CD171 (L1-CAM)*^{‡}, CEA*^{‡}, CSPG4*^{‡}, EPHA2*^{‡}, FAP*^{†‡}, LRRC15^{†‡}, FOLR1*^{†‡}, IL-13Rα*^{†‡}, Mesothelin*^{†‡}, MUC1*^{†‡}, MUC16*^{†‡}, TROP2*^{†‡}, claudin 18.2^{†‡}, and ROR1^{†‡} (associated with solid tumors). (* indicates that exemplary antibodies with the indicated specificity from which a binding moiety could be derived can be found in U.S. Pat. No. 11,326,182B2 Table 9 or 10. ^{†} indicates that exemplary antibodies with the indicated specificity from which a binding moiety could be derived can be found in Wilkinson & Hale, 2022. Both references cited and incorporated by reference above. ^{‡} indicates that exemplary antibodies with the indicated specificity from which a binding moiety could be derived can be found in the Therapeutic Antibody Database (TABS) at tabs.craic.com. Other suitable antibodies can be found in Appendix A which is incorporated herein by reference for all that it teaches about individual antibodies and the antigens they bind. Many of these target antigens are themselves receptors that could bind to their ligand if expressed on an immune cell. Accordingly, in some embodiments, the extracellular binding domain of the CAR comprises a ligand of a receptor expressed on the target cell. In still further embodiments, the extracellular binding domain of the CAR comprises a ligand binding domain of a receptor for a ligand expressed on the target cell. The advantages of the aspects and embodiments disclosed herein are independent of the specificity of the binding moiety. As such, the disclosed aspects and embodiments are generally agnostic to binding specificity. In certain embodiments, a particular binding specificity can be required.

In some embodiments, the tLNP comprises a nucleic acid encoding an anti-CD19 chimeric antigen receptor (CAR). In some embodiments, the nucleic acid comprises mRNA. Examples of anti-CD19 CARs include those incorporating a CD19 binding moiety derived from the human antibody 47G4 or the mouse antibody FMC63. FMC63 and the derived scFv have been described in Nicholson et al., Mol. Immun. 34(16-17):1157-1165 (1997) and PCT Application Publication Nos. WO 2018/213337 and WO 2015/187528, the entire contents of each of which are incorporated by reference herein for all that they teach about anti-CD19 CARs and their use. CAR based on 47G4 are disclosed in U.S. Pat. No. 10,287,350 which is incorporated by reference herein for all that it teaches about anti-CD19 CARs and their use. In some instances, the anti-CD19 CAR is the CAR found in tisagenlecleucel, lisocabtagene maraleucel, axicabtagene ciloleucel, or brexucabtagene autoleucel. Each of these CARs constitutes means for targeting an immune cell, for example, a T cell, to CD19. The entire contents of each of foregoing references in this paragraph are incorporated by reference for all that they teach about the design, structure, and activity of anti-CD19 CARs. In any of the aforementioned tLNP embodiments, certain embodiments include tLNPs encapsulating a CD19 CAR payload encoded by RNA and having a T cell targeting moiety, such as an anti-CD8 antibody.

In some embodiments, the tLNP comprises a nucleic acid encoding an anti-CD20 chimeric antigen receptor (CAR). CD20 is an antigen found on the surface of B cells as early as the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the nucleic acid comprises mRNA. Examples of anti-CD20 CARs include those incorporating a CD20 binding moiety derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In some embodiments, the anti-CD20 CAR is derived from a CAR specific to CD20, including, for example, MB-106 (Fred Hutchinson Cancer Research Center, see Shadman et al., Blood 134(Suppl.1):3235 (2019)) UCART20 (Cellectis, www.cellbiomedgroup.com), or C-CAR066 (Cellular Biomedicine Group, see Liang et al., J. Clin. Oncol. 39(15) suppl:2508 (2021)). In some embodiments, the extracellular binding domain of the anti-CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). Each of these CARs constitutes means for targeting an immune cell, for example, a T cell, to CD20. The entire contents of each of foregoing references in this paragraph are incorporated by reference for all that they teach about the design, structure, and activity of anti-CD20 CARs. In any of the aforementioned tLNP embodiments, certain embodiments include tLNPs encapsulating a CD20 CAR payload encoded by RNA and having a T cell targeting moiety, such as an anti-CD8 antibody.

In some embodiments, the tLNP comprises a nucleic acid encoding an anti-BCMA chimeric antigen receptor (CAR). BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the nucleic acid comprises mRNA. Examples of anti-BCMA CARs include those incorporating a BCMA binding moiety derived from C11D5.3, a Mouse monoclonal antibody as described in Carpenter et al., Clin. Cancer Res. 19(8):2048-2060 (2013). See also PCT Application Publication No. WO 2010/104949. In some embodiments, the extracellular binding domain of the BCMA CAR comprises an scFv derived from another Mouse monoclonal antibody, C12A3.2, as described in Carpenter et al., Clin. Cancer Res. 19(8):2048-2060 (2013) and PCT Application Publication No. WO02010104949. In some embodiments, the extracellular binding domain of the BCMA CAR comprises an scFv derived from a Mouse monoclonal antibody with high specificity to human BCMA, referred to as BB2121 in Friedman et al., Hum. Gene Ther. 29(5):585-601 (2018). See also, PCT Application Publication No. WO02012163805. In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO 2018/028647. In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO 2019/006072. In some embodiments, the extracellular binding domain of the BCMA CAR comprises an scFv derived from CT103A (or CAR0085) as described in U.S. Pat. No. 11,026,975 B2. Further anti-BCMA CARs are disclosed in U.S. Application Publication Nos. 2020/0246381 and 2020/0339699. Further anti-BCMA CARs include Allo-605 (described in U.S. Patent Publication No. 20200261503), CT053 (described in U.S. Pat. No. 11,525,006), Descartes-08 (described in U.S. Pat. No. 10,934,337), LCAR-B38M (described in U.S. Pat. No. 10,934,363), PersonGen anti-BCMA CAR (described in CN114763383), Pregene Bio anti-BCMA CAR (described in U.S. Patent Publication No. US20220218746), the CAR in ciltacabtagene autoleucel (binding moiety described in US20170051068), and the CAR in idecabtagene vicleucel (described in U.S. Pat. No. 10,383,929). Each of these CARs constitutes means for targeting an immune cell, for example, a T cell, to BCMA. Further antibodies comprising an anti-BCMA antigen binding domains that can be used in construction a CAR include AMG224 (described with other anti-BCMA antibodies in U.S. Pat. No. 9,243,058), EMB-06 (described with other anti-BCMA antibodies in U.S. Patent Publication No. US20230002489), HPN217 (described in U.S. Pat. No. 11,136,403), MED12228 (described in U.S. Pat. No. 10,988,546), REGN5459 (described in U.S. Pat. No. 11,384,153), SAR445514 (described in U.S. Patent Publication No. 20240034816), SEA-BCMA (described in U.S. Pat. No. 11,078,291), TNB-383B (described in U.S. Pat. No. 11,505,606), TQB2934 (described in U.S. Patent Publication No. 20230193292), WV078 (described in U.S. Pat. No. 11,492,409), alnuctamab (described in U.S. Pat. No. 10,683,369), belantamab (described in U.S. Pat. No. 9,273,141), elranatamab (described in U.S. Pat. No. 11,814,435), ispectamab (described in U.S. Patent Publication No. 20210130483), linvoseltamab (described in U.S. Pat. No. 11,919,965), pavurutamab (described in U.S. Pat. No. 11,419,933), and teclistamab (described in U.S. Pat. No. 10,072,088). A bispecific anti-BCMA, anti-CD19 CAR is described in WO02022007650. The entire contents of each of foregoing references in this paragraph are incorporated by reference for all that they teach about the design, structure, and activity of anti-BCMA CARs and anti-BCMA antibodies that can provide an antigen binding domain for a CAR or immune cell engager. In any of the aforementioned tLNP embodiments, certain embodiments include tLNPs encapsulating a BCMA CAR payload encoded by RNA and having a T cell targeting moiety, such as an anti-CD8 antibody.

In some embodiments, the tLNP comprises a nucleic acid encoding an anti-GPRC5D chimeric antigen receptor (CAR). GPRC5D is a G protein-coupled receptor without known ligands and of unclear function in human tissue. However, this receptor is expressed in myeloma cell lines and in bone marrow plasma cells from patients with multiple myeloma. GPRC5D has been identified as an immunotherapeutic target in multiple myeloma and Hodgkin lymphomas. Examples of anti-GPRC5D CARs include those incorporating a GPRC5D binding moiety such as MCARH109 (Mailankody et al., N Engl J Med. 387(13): 1196-1206 (2022)), BMS-986393, or OriCAR-017 (Rodriguez-Otero et al., Blood Cancer J. 14(1): 24 (2024)). Examples of anti-GPRC5D CARs include those incorporating a GPRC5D binding moiety derived from an antibody specific to GPRC5D, for example, talquetamab (Pillarisetti et al., Blood 135:1232-43 (2020)), or forimtamig. In some embodiments, the extracellular binding domain of the anti-GPRC5D CAR comprises an scFv derived from a 6D9 Mouse antibody with specificity to human GPRC5D (see creative-biolabs.com/car-t/anti-gprc5d-6d9-h-41bb-cd3-car-pcdcar1-26380.htm). In some embodiments, the extracellular binding domain of the GPRC5D CAR comprises an scFv of anti-GPRC5D antibody linked to 4-1BB or CD28 costimulatory domain and CD3ζ (signaling domain as described in Mailankody et al., N Engl J Med. 387(13): 1196-1206 (2022); creative-biolabs.com/car-t/anti-gprc5d-6d9-h-41bb-cd3-car-pcdcar1-26380.htm; and Rodriguez-Otero et al., Blood Cancer J. 14(1): 24 (2024). The entire contents of each of foregoing references in this paragraph are incorporated by reference for all that they teach about the design, structure, and activity of anti-GPRC5D CARs and anti-GPRC5D antibodies that can provide an antigen binding domain for a CAR or immune cell engager, and each example constitutes a means for binding GPRC5D. In any of the aforementioned tLNP embodiments, certain embodiments include tLNPs encapsulating an anti-GPRC5D CAR payload encoded by RNA and having a T cell targeting moiety, such as an anti-CD8 antibody.

In some embodiments, the tLNP comprises a nucleic acid encoding an anti-FCRL5 chimeric antigen receptor (CAR). FCRL5 (Fc receptor-like 5), also known as FCRH5, BXMAS1, CD307, CD307E, and IRTA2, is a protein marker expressed on the surface of plasma cells in patients with multiple myeloma. Furthermore, contact with FCRL5 stimulates B-cell proliferation; thus, FCRL5 has been identified as an immunotherapeutic target for this disease. Examples of anti-FCRL5 CARs include those incorporating an FCRL5 binding moiety, such as those described in WO2016090337, WO2017096120, WO2022263855, and WO2024047558. In some embodiments, the extracellular binding domain of the anti-FCRL5 CAR comprises an scFv with specificity to FCRL5, such as ET200-31, ET200-39, ET200-69, ET200-104, ET200-105, ET200-109, or ET200-117. In some embodiments, the extracellular binding domain of the anti-FCRL5 CAR comprises an scFv derived from a mouse antibody with specificity to human FCRL5. Such antibodies include 7D11, F25, F56, and F119, as described in Polson et al., Int. Immunol., 18(9): 1363-1373 (2006); Franco et al., J. Immunol. 190(11): 5739-5746 (2013); Ise et al., Clin. Cancer Res. 11(1): 87-96 (2005); and Ise et al., Clin. Chem. Lab. Med. 44(5): 594-602 (2006), all of which are incorporated by reference herein. In some embodiments, the extracellular binding domain of the anti-FCRL5 CAR comprises a binding moiety derived from the antigen binding domain of an anti-FCRL5 antibody or nanobody, including cevostamab, 2A10H7, 307307, 2A10D6, 13G9, 10A8, 509f6, EPR27365-87, EPR26948-19, or EPR26948-67, or as disclosed in WO2016090337, WO2017096120, WO2022263855, or WO02024047558. In some embodiments, the extracellular binding domain of the anti-FCRL5 CAR comprises a binding moiety derived from an antibody-drug conjugate targeting FCRL5, such as those described in Elkins et al., Mol. Cancer Ther. 11(10): 2222-2232 (2012). In some embodiments, the extracellular binding domain of the anti-FCRL5 CAR is linked to a costimulatory domain, such as a 4-1BB or CD28 costimulatory domain, and a signaling domain, such as a CD34 signaling domain. The entire contents of each of the foregoing references in this paragraph are incorporated by reference for all that they teach about the design, structure, properties, and activity of anti-FCRL5 CARs and anti-FCRL5 antibodies that can provide an antigen binding domain for a CAR or immune cell engager. Each example constitutes a means for binding FCRL5. In any of the aforementioned tLNP embodiments, certain embodiments include tLNPs encapsulating a FCRL5 CAR payload encoded by RNA and having a T cell targeting moiety, such as an anti-CD8 antibody.

In some embodiments, the tLNP comprises a nucleic acid(s) encoding one or more CARs that target multiple antigens. In some embodiments, the tLNP comprises distinct mRNAs that are encapsulated together in a single tLNP, with each mRNA encoding one monospecific CAR. For examples, the tLNP can comprise an mRNA encoding an anti-CD19 CAR and an mRNA encoding an anti-CD20 CAR, an mRNA encoding an anti-CD19 CAR and an mRNA encoding an anti-BCMA CAR, an mRNA encoding an anti-GPRC5D CAR and an mRNA encoding an anti-BCMA CAR, or an mRNA encoding an anti-FCRL5 CAR and an mRNA encoding an anti-BCMA CAR. In some embodiments, the tLNP comprises a single mRNA encoding a bicistronic mRNA encoding two monospecific CARs. For example, the bicistronic mRNA can encode an anti-CD19 CAR and an anti-CD20 CAR, an anti-CD19 CAR and an anti-BCMA CAR, an anti-GPRC5D CAR and an anti-BCMA CAR, or an anti-FCRL5 CAR and an anti-BCMA CAR. In some embodiments, the tLNP comprises a single mRNA encoding an mRNA encoding a multispecific CAR. In some embodiments, the tLNP comprises a single mRNA encoding an mRNA encoding a bispecific CAR. For example, the mRNA can encode an anti-CD19 and anti-CD20 bispecific CAR, an anti-CD19 and anti-BCMA bispecific CAR, an anti-GPRC5D and anti-BCMA bispecific CAR, or an anti-FCRL5 and anti-BCMA bispecific CAR. In some embodiments, multiple tLNPs can be co-formulated in a combination with each tLNP comprising one mRNA. In some instances, the one mRNA encodes one monospecific CAR. For examples, two tLNPs can be co-formulated with one tLNP comprising an mRNA encoding an anti-CD19 CAR and the other tLNP comprising an mRNA encoding an anti-CD20 CAR, one tLNP comprising an mRNA encoding an anti-C19 CAR and the other tLNP comprising an mRNA encoding an anti-BCMA CAR, one tLNP comprising an mRNA encoding an anti-GPRC5D CAR and the other tLNP comprising an mRNA encoding an anti-BCMA CAR, or one tLNP comprising an mRNA encoding an anti-FCRL5 CAR and the other tLNP comprising an mRNA encoding an anti-BCMA CAR. In some embodiments, multiple tLNPs can be co-administered in a combination, either simultaneously or sequentially, wherein each comprises one mRNA. In some instances, the one mRNA encodes one monospecific CAR. For examples, two tLNPs can be co-administered in a combination, either simultaneously or sequentially, with one tLNP comprising an mRNA encoding an anti-CD19 CAR and the other tLNP comprising an mRNA encoding an anti-CD20 CAR, one tLNP comprising an mRNA encoding an anti-C19 CAR and the other tLNP comprising an mRNA encoding an anti-BCMA CAR, one tLNP comprising an mRNA encoding an anti-GPRC5D CAR and the other tLNP comprising an mRNA encoding an anti-BCMA CAR, or one tLNP comprising an mRNA encoding an anti-FCRL5 CAR and the other tLNP comprising an mRNA encoding an anti-BCMA CAR. The targeting can be mediated by any of the CARs described herein. In addition to combinations of two specificities, higher order combinations are also possible, especially with the use of bi- and tri-specific CARs. Following these patterns, further embodiments are constituted mutatis mutandis by other tLNP or combinations of tLNPs comprising a nucleic acid(s) encoding one or more CARs that target multiple antigens involving these and other CAR specificities disclosed herein.

Cellular therapy involving the administration of genetically engineered cells to a patient has generally required depleting or ablative conditioning to facilitate engraftment of the engineered cells (for example, T cells or HSC). In the context of in vivo engineering and reprogramming such conditioning would be counterproductive as the conditioning would eliminate the very cells that are to be engineered. Instead, one can utilize activating and/or adjuvant conditioning to increase the number of cells amenable to engineering, to mobilize them to the locus of pathology, to make the locus of pathology (for example, a tumor microenvironment) more susceptible to treatment, to augment the therapeutic effect, etc., as appropriate for the particular disease and primary treatment. Conditioning agents include biological response modifiers (BRMs) that can be delivered directly to a subject or encoded in nucleic acid molecules, including as mRNA, and delivered to a subject using the LNP and tLNP compositions and formulations disclosed herein.

Accordingly, certain aspects are methods of conditioning a subject who receives an engineering agent comprising providing a tLNP comprising a nucleic acid molecule encoding a conditioning agent to the subject prior to, concurrently with, or subsequent to administration of the engineering agent. In various embodiments, an encoded conditioning agent comprises a γ-chain receptor agonist, an inflammatory chemokine, a pan-activating cytokine, an antigen presenting cell activity enhancer, an immune checkpoint inhibitor, or an anti-CCR4 antibody. In some embodiments, the γ-chain receptor cytokine comprises IL-15, IL-2, IL-7, or IL-21. In some embodiments, the immune checkpoint inhibitor comprises an anti-CTLA-4, anti-PD-1, anti-PD-1, anti-Tim-3, or anti-LAG-3 antibody. In some embodiments, the inflammatory chemokine comprises CCL2, CCL3, CCL4, CCLS, CCL11, CXCL1, CXCL2, CXCL-8, CXCL9, CXCL10, or CXCL11. In some embodiments, the antigen presenting cell activity enhancer comprises Flt-3 ligand, gm-CSF, or IL-18. In some embodiments, a pan-activating cytokine comprises IL-12 of IL 18. In certain embodiments, a conditioning agent comprises a transcription factor, for example, one selected from the group consisting of nuclear factor of activated T-cells (NFAT), NF-κB, T-bet, signal transducer and activator of transcription 4 (STAT4), Blimp-1, c-Jun, and Eomesodermin (Eomes) and the tLNP is targeted to a T cell. In some embodiments, a tLNP encapsulating the nucleic acid-encoded conditioning agent is administered systemically, for example, by intravenous or subcutaneous infusion or injection. In other embodiments, the tLNP is administered locally, for example, by intralesional or intraperitoneal injection or infusion. In some embodiments, nucleic acid molecules encoding the conditioning agent and the engineering agent are encapsulated in the same tLNP while in other embodiments they are encapsulated in separate tLNPs. These two modes of delivery of conditioning agents are described in greater detail in PCT application PCT/US 2023/072426, which is incorporated by reference for all that it teaches about conditioning agents and their delivery of LNPs or tLNPs that is not inconsistent with the present disclosure. In some embodiments, the nucleic acid comprises mRNA.

The term “treating” or “treatment” broadly includes any kind of treatment activity, including the mitigation, cure or prevention of disease, or aspect thereof, in man or other animals, or any activity that otherwise affects the structure or any function of the body of man or other animals. Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. In some embodiments, the orders, instructions, and advice aspect of treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians group, and the like. All such orders, instructions, and advice are to be seen as conditioning receipt of the benefit of the treatment on compliance with the instruction. In some instances, a financial benefit is also received by the patient for compliance with such orders, instructions, and advice. In some instances, a financial benefit is also received by the healthcare professional for compliance with such orders, instructions, and advice.

Some embodiments of these methods of treatment comprise administration of an effective amount of a compound or a composition disclosed herein. Some instances relate to a therapeutically (or prophylactically) effective amount. A therapeutically effective amount is not necessarily a clinically effective amount, that is, while there can be therapeutic benefit as compared to no treatment, a method of treatment may not be equivalent or superior to a standard of care treatment existing at some point in time. Other instances relate to a pharmacologically effective amount, that is an amount or dose that produces an effect that correlates with or is reasonably predictive of therapeutic (or prophylactic) utility. As used herein, the term “therapeutically effective amount” is synonymous with “therapeutically effective dose” and means at least the minimum dose of a compound or composition disclosed herein necessary to achieve the desired therapeutic or prophylactic effect. Similarly, a pharmacologically effective dose means at least the minimum dose of a compound or composition disclosed herein necessary to achieve the desired pharmacologic effect. Some embodiments refer to an amount sufficient to prevent or disrupt a disease process, or to reduce the extent or duration of pathology. Some embodiments refer to a dose sufficient to reduce a symptom associated with the disease or condition being treated.

The following examples are intended to illustrate various embodiments. As such, the specific embodiments discussed are not to be constructed as limitations on the scope of this disclosure. It is apparent to one skilled in the art that various equivalents, changes, and modifications can be made without departing from the scope of this disclosure, and it is understood that such equivalent embodiments are to be included herein. Further, all references cited in the disclosure are hereby incorporated by reference in their entirety, as if fully set forth herein.

### EXAMPLES

### Example 1: Synthesis of 2-(2-(tert-butoxy)-2-oxoethyl)propane-1,3-diyl dinonanoate (1)

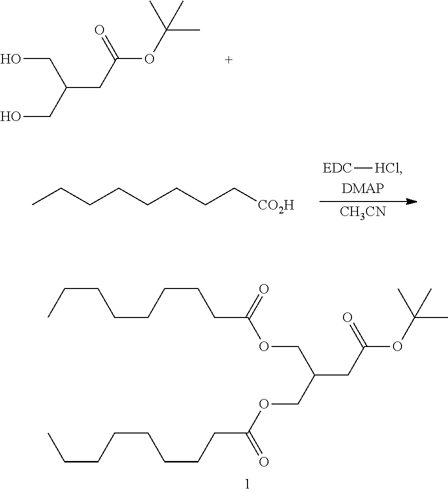

To a solution of tert-butyl 4-hydroxy-3-(hydroxymethyl)butanoate (Org. Proc. Res. Dev. 2011, 15, 515; 44.0 g, 0.231 mol), in acetonitrile (900 mL), cooled in an ice water bath under nitrogen, was added nonanoic acid (76.86 g, 0.486 mol), followed by the addition of DMAP (28.22 g, 0.231 mol) and EDC-HCl (97.8 g, 0.513 mol). The mixture was stirred for 1 hour, then was allowed to warm to room temperature and was stirred for 12 hours. The solution was cast into n-heptane (1.40 L) and water (0.9 L) and the organic phase was separated. The organic phase was washed twice with MeOH:10% aq. citric acid (0.90 L), followed by washing twice with a mixture of MeOH:H2O:triethyl amine (0.90 L, 3:1:0.1). The organic phase was then washed with 10% aq. NaCl, dried over Na₂SO₄, filtered, and concentrated in vacuo to provide 2-(2-(tert-butoxy)-2-oxoethyl)propane-1,3-diyl dinonanoate 1 (90.20 g, 96.3% purity by HPLC, 0.185 mol, 80% yield) as a pale yellow viscous liquid.

¹H-NMR (300 MHz, CDCl₃): δ=4.12 (m, 4H), 2.53 (m, 1H), 2.29-2.34 (6H), 1.52-1.64 (4H), 1.46 (s, 9H), 1.16-1.37 (24H), 0.89 (t, J=7.0 Hz, 6H); LCMS: RT=1.748, calcd. for C₂₇H₅₀O₆ minus t-butyl+H⁺: 415.31. Found: 415.20.

### Example 2: Synthesis of 4-(nonanoyloxy)-3-((nonanoyloxy)methyl)butanoic acid (2)

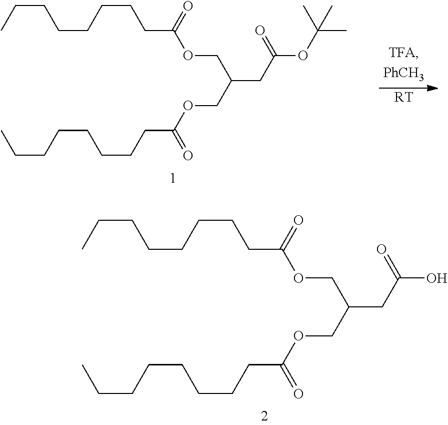

To a solution of 1 (90.0 g, 96.3% purity, 0.184 mol) in toluene (0.41L), cooled in an ice-water bath under nitrogen, was added TFA (208.46 g, 1.83 mol, 140 mL) over a period of 30 minutes. After the addition was complete the mixture was warmed to 15° and the mixture was stirred for 18 hours. The chilled solution was cast into n-heptane (1.50L) and the resulting solution was extracted with 5% aq. Potassium phosphate (2.0L) and the aqueous phase was collected. The organic phase was extracted with MeOH:H2O:triethyl amine (2.0L, 5:1:0.1), and the combined aq. phases were cast into n-heptane (1.80L) and 1.2M aq. HCl (1.0L). The organic layer was separated, washed with MeOH:water (1.0L, 1:1), dried over Na₂SO₄, filtered and concentrated in vacuo to afford acid 2 (69.0 g, 96.1% purity by HPLC, 0.177 mol, 96% yield) as a pale yellow, viscous oil.

¹H-NMR (300 MHz, CDCl₃): δ=4.13 (m, 4H), 2.58 (m, 1H), 2.48 (m, 2H), 2.32 (m, 4H), 1.63 (m, 4H), 1.20-1.37 (24H), 0.89 (t, J=7.0 Hz, 6H); LCMS: RT=1.723, Calcd. for C₂₃H₄₂O₆+H⁺ 415.31. Found 415.30.

### Example 3: Synthesis of rel-((2S,4R)-1-benzylazetidine-2,4-diyl)dimethanol (3)

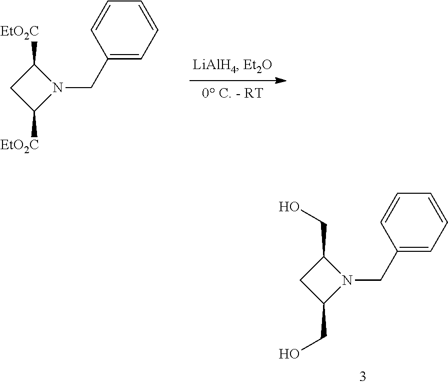 [SMILES: CCOC(=O)[C@H]1C[C@@H](C(=O)OCC)N1Cc1ccccc1 ; OC[C@@H]1C[C@H](CO)N1Cc1ccccc1]

To a solution of commercially available diethyl rel-(2S,4R)-1-benzylazetidine-2,4-dicarboxylate (10.0 g, 34.32 mmol) in ether (200 mL), cooled in an ice-water bath under nitrogen, was added LiAlH4 (2.74 g, 74.08 mmol) in portions over a period of 15 minutes. The cooling bath was removed, and the mixture was allowed to warm to room temperature and was stirred for 5 hours. The mixture was cooled in an ice-water bath and the reaction was carefully quenched by the of ice-water (200 mL). The organic phase was separated, the aqueous phase was extracted with EtOAc (3×300 mL), the combined organic phases were washed with brine (2×300 mL), dried over Na₂SO₄, filtered and concentrated in vacuo to provide crude 3 (4.00 g, 83.3% HPLC purity, 16.1 mmol, 47% yield) as a yellow viscous liquid.

¹H-NMR (300 MHz, CDCl₃): δ=7.26-7.33 (5H), 4.03 (m, 1H), 3.62-3.77 (3H), 3.49-3.63 (4H), 2.26 (brs, 2H), 2.13 (m, 2H); LCMS: RT=0.91, Calcd. for C₁₂H₁₇NO₂+H⁺ 208.13. Found 208.10.

### Example 4: Synthesis of tert-butyl rel-(2S,4R)-2,4-bis(hydroxymethyl)azetidine-1-carboxylate (4)

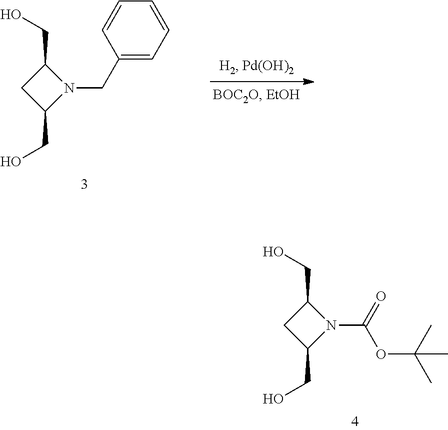

To a solution of 3 (4.00 g, 16.1 mmol) in EtOH (200 mL) was added Pd(OH)₂ (0.80 g, 20% w/w and BOC₂0 (5.27 g, 2.15 mmol), and the mixture was placed under 30 psi of hydrogen. The mixture was shaken under 30 psi of hydrogen for 20 hours, then the catalyst was removed by filtration through a pad of Celite, the filter cake was rinsed with EtOH (50 mL) and the combined filtrates were concentrated in vacuo to give crude 4 as a yellow oil. Crude 4 was purified by chromatography on a column of silica gel (250 g, 100-200 mesh), packed and eluted with petroleum ether-EtOA (4:1). Fractions containing 4 were pooled and concentrated in vacuo to furnish 4 (2.10g, HPLC purity 97.9%, 9.20 mmol, 57% yield) as a clear, pale yellow, oil.

¹H-NMR (300 MHz, CDCl₃): δ=4.31 (m, 2H), 3.80 (dd, J=11.7, 2.7 Hz, 2H), 3.67 (dd, J=11.7, 5.7 Hz, 2H), 2.93 (brs, 2H), 2.22 (m, 1H), 1.94 (m, 1H), 1.49 (s, 9H).

### Example 5: Synthesis of ((((rel-(2S,4R)-1-(tert-butoxycarbonyl)azetidine-2,4-diyl)bis(methylene))bis(oxy))bis(2-oxoethane-2,1-diyl))bis(propane-2,1,3-triyl) tetranonanoate (5)

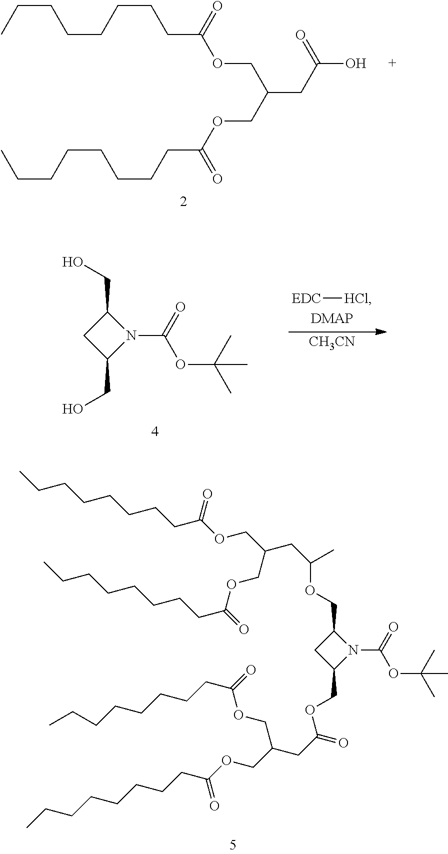

To solution of 4 (2.00 g, 9.205 mmol) in dichloromethane (100 mL), at room temperature under nitrogen, was added in order 2 (8.01 g, 19.331 mmol), DMAP (1.12 g, 9.205 mmol) and EDC-HCl (4.41 g, 23.012 mmol). The reaction mixture was stirred for 18 hours at room temperature, then was concentrated in vacuo and the residue was dissolved in n-heptane (500 mL). The resulting solution was washed with MeOH/water (10:1, 2×200 mL) and the organic phase was concentrated in vacuo to provide crude 5. Crude 5 was purified by chromatography on a column of silica gel (500 g, 100-200 mesh, packed with n-heptane, eluted with a gradient of n-heptane:EtOAc 100:0 to 90:10). Qualified fractions were combined and concentrated in vacuo to give 5 (7.00g, HPLC purity 97.33%, 6.93 mmol, 75.3% yield) as a light-yellow oil.

¹H-NMR (400 MHz, CDCl₃): δ=4.25-4.37 (6H), 4.18 (m, 8H), 2.62 (m, 2H), 2.50 (m, 4H), 2.42 (m, 1H), 2.33 (t, J=7.0 Hz, 8H), 1.83 (m, 1H), 1.52-1.60 (8H), 1.45 (s, 9H), 1.18-1.37 (40H), 0.89 (t, J=7.9 Hz, 12H); LCMS: RT=2.24, Calcd. for C₅₆H₉₉NO₁₄+Na+ 1032.70, Found 1032.70.

### Example 6: Synthesis of Rel-(2S,4R)-2,4-bis(((4-(nonanoyloxy)-3-((nonanoyloxy)methyl)butanoyl)oxy)methyl)azetidin-1-ium trifluoroacetate (6)

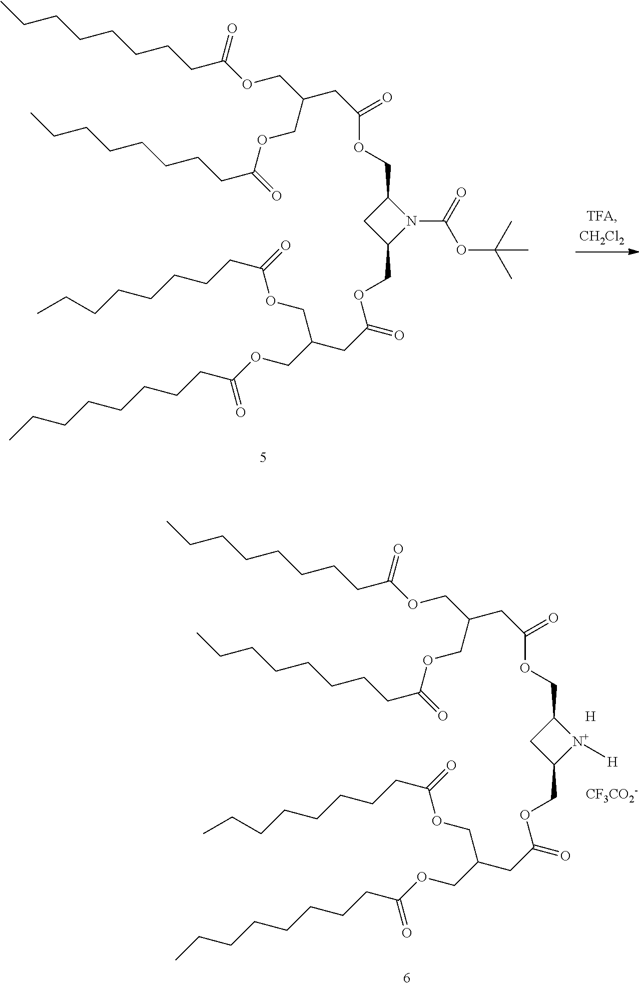

To a solution of 5 (7.00 g, 6.928 mmol) in dichloromethane (35 mL), at room temperature under nitrogen, was added trifluoroacetic acid (14 mL, 20.85 g, 0.183 mol) over a period of 10 minutes. The mixture was allowed to stir for 3 hours at room temperature, then was concentrated in vacuo to afford crude 6. Ammonium salt 6 was dissolved in n-heptane (500 mL) and solution was washed with brine (2×200 mL), water (200 mL), then the solution was concentrated in vacuo to afford 6 as a light-yellow oil (6.80g, HPLC purity 91.82%).

¹H-NMR (400 MHz, CDCl₃): δ=4.77 (brs, 2H), 4.44 (m, 4H), 4.02-4.25 (9H), 2.72 (brm, 1H), 2.38-2.5 (8H), 2.33 (m, 8H), 1.51-1.62 (8H), 1.20-1.37 (40H), 0.88 (m, 12H); LCMS: RT=1.72, Calcd. for C₅₁H₉₂NO₁₂ 910.55. Found 910.70.

### Example 7: Synthesis of ((((rel-(2S,4R)-1-(1H-Imidazole-1-carbonyl)azetidine-2,4-diyl)bis(methylene))bis(oxy))bis(2-oxoethane-2,1-diyl))bis(propane-2,1,3-triyl) tetranonanoate (7)

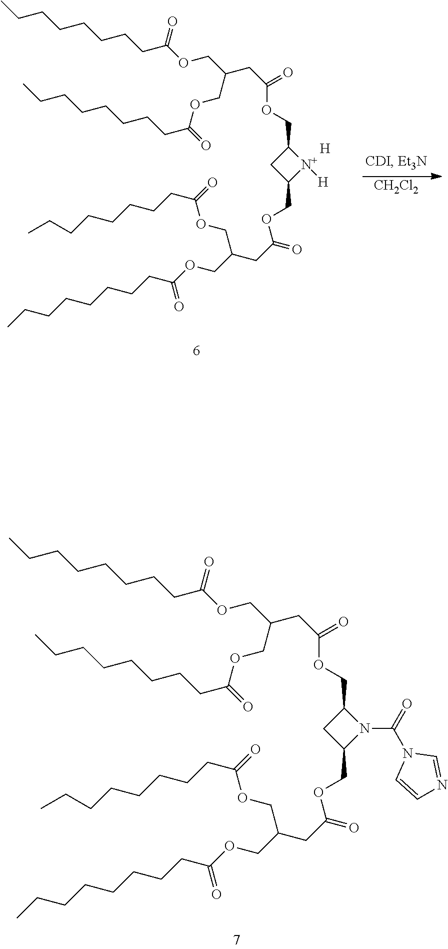

To a solution of 6 (6.50 g, 91.82% pure by HPLC) in dichloromethane (180 mL), cooled in an ice-water bath under nitrogen, was added CDI (10.44 g, 64.44 mmol) and Et3N (3.26 g, 32.27 mmol), the mixture was allowed to stir for 1 hour, then was warmed to room temperature and stirred for 18 hours. The reaction was quenched with 0.5M aq. HCl (150 mL) and the organic phase was separated. The aqueous layer was extracted with dichloromethane (2×300 mL) and the combined organic phases were concentrated in vacuo and the residual oil was dissolved in n-heptane (300 mL). The n-heptane solution was washed with MeOH/water (5:1, 2×200 mL) and the solvent was removed in vacuo to provide 7 (6.00g, HPLC purity 97.24%, 5.97 mmol, 92% yield over 2 steps) as a light-yellow oil.

¹H-NMR (400 MHz, CDCl₃): δ=8.09 (s, 1H), 7.39 (s, 1H), 7.11 (s, 1H), 4.52 (m, 2H), 4.44 (m, 4H), 4.12 (m, 8H), 2.75 (m, 1H), 2.57 (m, 2H), 2.43 (m, 4H), 2.28 (m, 8H), 2.07 (m, 1H), 1.52-1.64 (8H), 1.18-1.32 (40H), 0.88 (t, J=7.20 Hz, 12H); LCMS: RT=1.74, Calcd. for C₅₅H₉₃N₃O₁₃+H⁺ 1004.68, Found 1004.70.

### Example 8: Synthesis of ((((rel-(2S,4R)-1-((2-(dimethylamino)ethoxy)carbonyl)azetidine-2,4-diyl)bis(methylene))bis(oxy))bis(2-oxoethane-2,1-diyl))bis(propane-2,1,3-triyl) tetranonanoate (CICL-221)

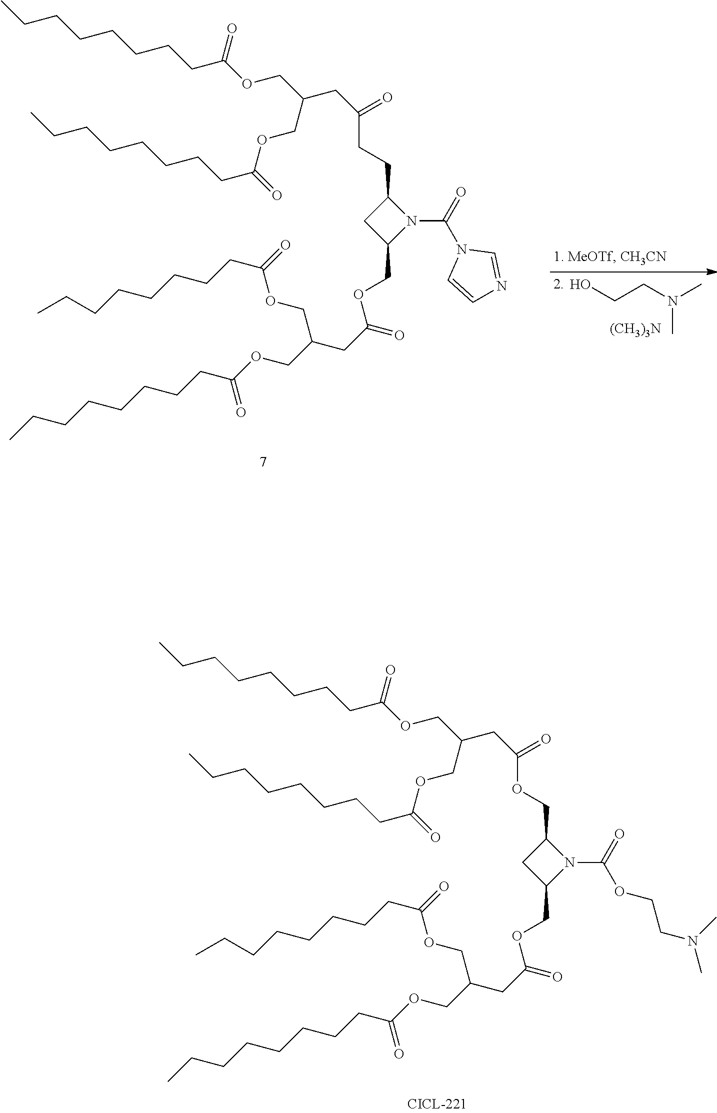

To a solution of 7 (5.80 g, 5.775 mmol), in CH3CN (120 mL), cooled in an ice-water bath under nitrogen was added methyl trifluoromethanesulfonate (MeOTf, 1.14 g, 6.947 mmol) over a period of 5 minutes. The mixture was allowed to stir for 2 hours at 0° C., then a solution of trimethylamine in CH₃CN (2.4M, 11.55 mL, 27.7 mmol) was added over a period of 10 minutes followed by the addition of 2-dimethylaminoethanol (0.77 g, 8.638 mmol) in one portion. The mixture was stirred for 1 hour at 0° C., then, was warmed to 60° C. and stirred for 120 hours. The reaction mixture was concentrated in vacuo and the residue was dissolved in n-heptane (400 mL). The solution was washed with MeOH/water (5:1, 2×150 mL), and the organic phase was dried over Na₂SO₄. Filtration afforded a heptane solution of crude CICL-221 to which was added silica gel (15g, type: ZCX-2, 100-200 mesh). The solvent was removed in vacuo and the impregnated silica gel was placed atop a combi-flash column of silica gel (150g, type: ZCX-2, 100-200 mesh). The column was eluted with a gradient of n-heptane:ethyl acetate from 100:0 to 35:65. Qualified fractions (eluted at 50:50) were combined and concentrated in vacuo to provide CICL-221 (1.876 g, 1.83 mmol, 32%) as a pale yellow oil.

¹H-NMR (400 MHz, CDCl₃): δ=4.27 (m, 8H), 4.13 (m, 8H), 2.58 (m, 4H), 2.40-2.52 (5H), 2.23-2.34 (14H), 1.89 (m, 1H), 1.60 (m, 8H), 1.18-1.38 (40H), 0.88 (t, J=6.90 Hz, 12H); HPLC (RT=20.11) 96.57% purity; LCMS: RT=1.215, Calcd. for C₅₆H₁₀₀N₂O₁₄+H⁺ 1025.73. Found 1025.70.

### Example 9: Synthesis of tert-butyl rel-(2R,4R)-2,4-bis(hydroxymethyl)azetidine-1-carboxylate (9)

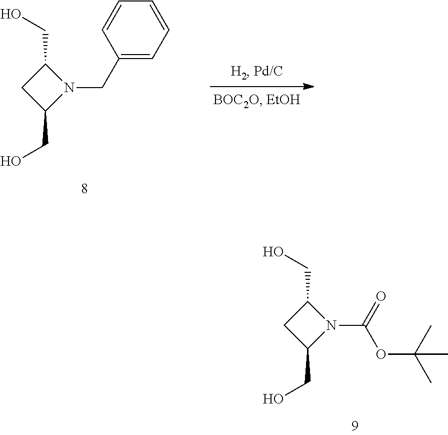

To a solution of 8 (7.90 g, 38.11 mmol, Tetrahedron Asymmetry 2001, 12, 605) in EtOH (240 mL) was added 10% Pd/C (1.60 g, 20% w/w and BOC2O (12.48 g, 57.16 mmol), and the mixture was placed under 30 psi of hydrogen. The mixture was shaken under 30 psi of hydrogen for 30 hours, then the catalyst was removed by filtration through a pad of Celite, the filter cake was rinsed with EtOH (100 mL) and the combined filtrates were concentrated in vacuo to give crude 9 as a yellow oil. Crude 9 was purified by chromatography on a column of silica gel (300 g, 100-200 mesh), packed and eluted with petroleum ether-EtOA (4:1). Fractions containing 9 were pooled and concentrated in vacuo to furnish 9 (6.90g, HPLC purity 98.05%, 31.63 mmol, 83% yield) as a clear, pale yellow, oil.

¹H-NMR (300 MHz, CDCl₃): δ=4.58-4.23 (3H), 3.93-3.62 (4H), 2.32 (brs, 1H), 2.00 (brm, 2H), 1.47 (s, 9H); LCMS: RT=0.897, Calcd. for C₁₀H₁₉NO₄+H⁺ 218.14. Found 218.10.

### Example 10: Synthesis of ((((rel-(2R,4R)-1-(tert-butoxycarbonyl)azetidine-2,4-diyl)bis(methylene))bis(oxy))bis(2-oxoethane-2,1-diyl))bis(propane-2,1,3-triyl) tetranonanoate (10)

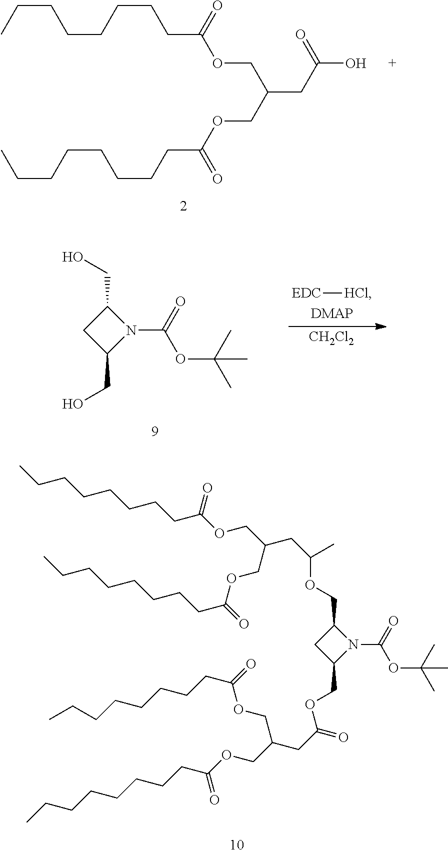

To solution of 9 (2.00 g, 9.205 mmol) in dichloromethane (100 mL), at room temperature under nitrogen, was added in order 2 (8.01 g, 19.331 mmol), DMAP (1.12 g, 9.205 mmol) and EDC-HCl (4.41 g, 23.012 mmol). The reaction mixture was stirred for 18 hours at room temperature, then was concentrated in vacuo and the residue was dissolved in n-heptane (500 mL). The resulting solution was washed with MeOH/water (10:1, 2×200 mL) and the organic phase was concentrated in vacuo to provide crude 10. Crude 10 was purified by chromatography on a column of silica gel (500 g, 100-200 mesh, packed with n-heptane, eluted with a gradient of n-heptane:EtOAc 100:0 to 90:10). Qualified fractions were combined and concentrated in vacuo to give 10 (7.00g, HPLC purity 95.94%, 6.93 mmol, 75.3% yield) as a light-yellow oil.

¹H-NMR (300 MHz, CDCl₃): δ=4.20-4.52 (5H), 4.13 (m, 8H), 2.60 (m, 2H), 2.46 (m, 4H), 2.30 (t, J=7.5 Hz, 8H), 2.19 (m, 2H), 1.52-1.65 (9H), 1.40 (s, 9H), 1.18-1.30 (40H), 0.88 (m, 12H); LCMS: RT=2.235, Calcd. for C₅₆H₉₉NO₁₄+Na+ 1032.70, Found 1032.80.

### Example 11: Synthesis of Rel-(2R,4R)-2,4-bis(((4-(nonanoyloxy)-3-((nonanoyloxy)methyl)butanoyl)oxy)methyl)azetidin-1-ium trifluoroacetate (11)

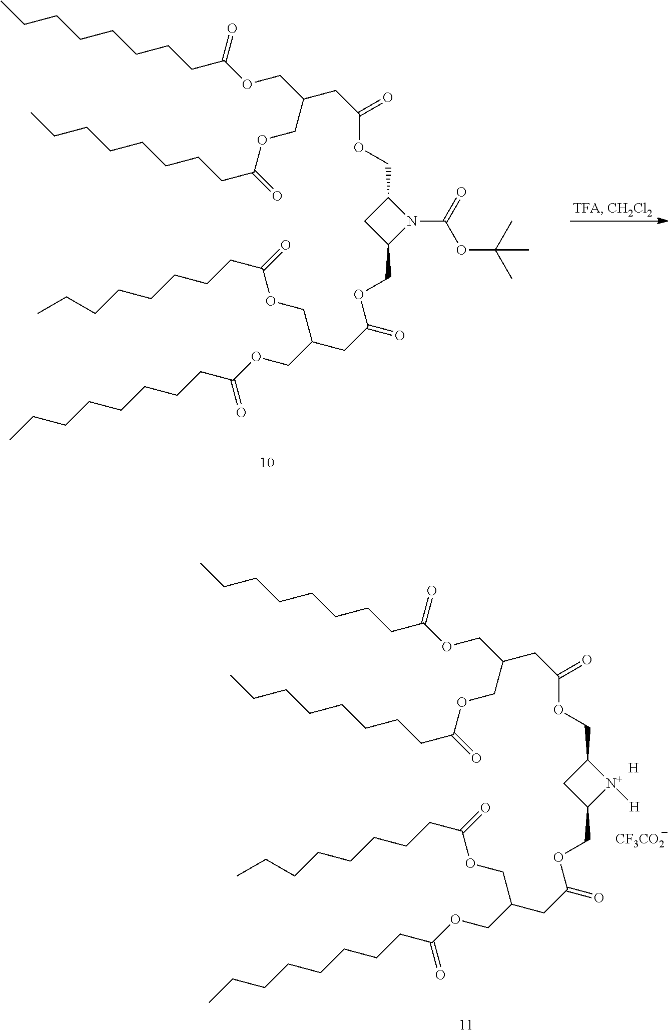

To a solution of 10 (7.00 g, 6.928 mmol) in dichloromethane (35 mL), at room temperature under nitrogen, was added trifluoroacetic acid (14 mL, 20.85 g, 0.183 mol) over a period of 10 minutes. The mixture was allowed to stir for 3 hours at room temperature, then was concentrated in vacuo to afford crude 11. Ammonium trifluoroacetate salt 11 was dissolved in n-heptane (500 mL) and solution was washed with brine (2×200 mL), water (200 mL), then the solution was concentrated in vacuo to afford 6 as a light-yellow oil (6.80g, HPLC purity 91.63%).

¹H-NMR (300 MHz, CDCl₃): δ=4.66 (brm, 2H), 4.46 (m, 4H), 4.16 (m, 8H), 2.42-2.65 (9H), 2.30 (t, J=7.4 Hz, 8H), 1.55-1.65 (9H), 1.20-1.40 (40H), 0.88 (m, 12H); LCMS: RT=1.724, Calcd. for C₅₁H₉₂NO₁₂ 910.66, Found 910.70.

### Example 12: Synthesis of ((((rel-(2R,4R)-1-(1H-Imidazole-1-carbonyl)azetidine-2,4-diyl)bis(methylene))bis(oxy))bis(2-oxoethane-2,1-diyl))bis(propane-2,1,3-triyl) tetranonanoate (12)

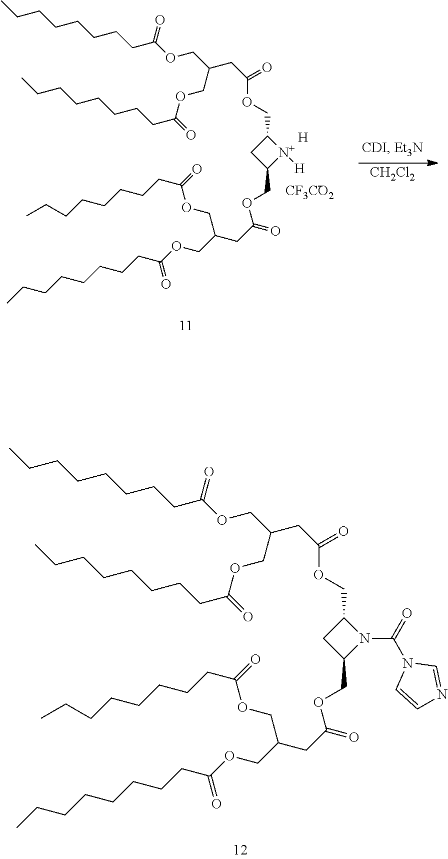

To a solution of 11 (6.50 g, 91.63% pure by HPLC) in dichloromethane (180 mL), cooled in an ice-water bath under nitrogen, was added CDI (10.44 g, 64.44 mmol) and Et3N (3.26 g, 32.27 mmol), the mixture was allowed to stir for 1 hour, then was warmed to room temperature and stirred for 18 hours. The reaction was quenched with 0.5M aq. HCl (150 mL) and the organic phase was separated. The aqueous layer was extracted with dichloromethane (2×300 mL) and the combined organic phases were concentrated in vacuo and the residual oil was dissolved in n-heptane (300 mL). The n-heptane solution was washed with MeOH/water (5:1, 2×200 mL) and the solvent was removed in vacuo to provide 12 (6.00g, HPLC purity 96.67%, 5.97 mmol, 93% yield over 2 steps) as a light-yellow oil.

¹H-NMR (300 MHz, CDCl₃): δ=0.87 (s, 1H), 7.32 (s, 1H), 7.12 (s, 1H), 4.85 (brm, 2H), 4.08-4.30 (12H), 2.57 (m, 2H), 2.26-2.51 (14H) 1.62 (m, 8H), 1.23-1.40 (40H), 0.89 (t, J=6.9 Hz, 12H); LCMS: RT 1.743, Calcd. for C₅₅H⁹³N₃O₁₃+H⁺ 1004.68, Found 1004.70.

### Example 13: Synthesis of ((((rel-(2S,4R)-1-((2-(dimethylamino)ethoxy)carbonyl)azetidine-2,4-diyl)bis(methylene))bis(oxy))bis(2-oxoethane-2,1-diyl))bis(propane-2,1,3-triyl) tetranonanoate (CICL-222)

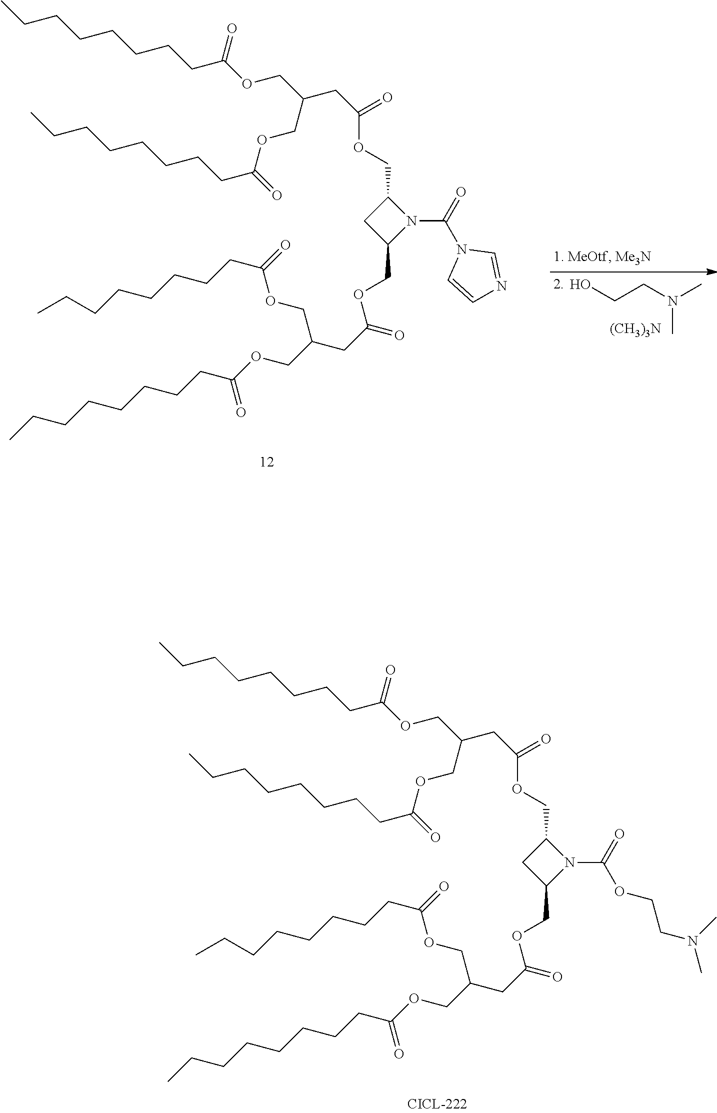

A solution of 12 (5.80 g, 5.78 mmol), in acetonitrile (120 mL) under nitrogen, was cooled in an ice water bath, then MeOTf (1.14 g, 6.95 mmol) was added over a period of 5 minutes. The mixture was stirred at 0° C. for 2 hours, then trimethylamine (11.55 mL, 2M in THF, 23.10 mmol) was added over a period of 5 minutes, followed by the addition of 2-dimethylaminoethanol (0.77 g, 8.64 mmol) in one portion. The resulting solution was stirred for 1 hour at 0° C., then was warmed to room temperature, followed by heating in a 75° C. oil bath for 8 days. After cooling to room temperature, the solvent was removed in vacuo and the residue was dissolved in n-heptane (400 mL). The n-heptane solution was washed with MeOH/water (5:1, 2×150 mL), and the organic phase was dried over Na₂SO₄. Filtration and concentration in vacuo yielded crude CICL-222, which was dissolved in CH₃CN (10 mL), and purified by preparative reverse phase HPLC (XB-phenyl column 19×250 mm, 5 μM; Mobile Phase A: water/0.1% TFA; Mobile Phase B: CH₃CN; flow rate 20 mL/min; Gradient 50% B to 90% B in 18 minutes; Wave Length 200 nM). Qualified fractions were concentrated in vacuo, to remove CH₃CN, and the pH of the aq. Residue was adjusted to 8.0 with 2% aq. Na₂CO₃. The aq. phase was extracted with n-heptane (3×100 mL), the combined organic phases were then washed with MeOH/water (5:1, 2×100 mL), water (200 mL), and dried (Na₂SO₄). Filtration and concentration in vacuo gave CICL-222 (1.26 g, 1.23 mmol, 97% purity by HPLC, 21% yield) as a clear, colorless, viscous oil.

¹H-NMR (300 MHz, CDCl₃): δ=4.31-4.57 (5H), 4.08-4.22 (11H), 2.61 (brm, 4H), 2.49 (m, 4H), 2.22-2.35 (16H), 1.63 (m, 8H), 1.20-1.40 (40H), 0.90 (t, J=6.6 Hz, 12H); LCMS: RT 1.588, Calcd. for C₅₆H₁₀₀N₂O₁₄+H⁺ 1025.73. Found 1025.70.

### Example 14: Synthesis of 2-(2-(((2S,4R)-1-(tert-butoxycarbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (14)

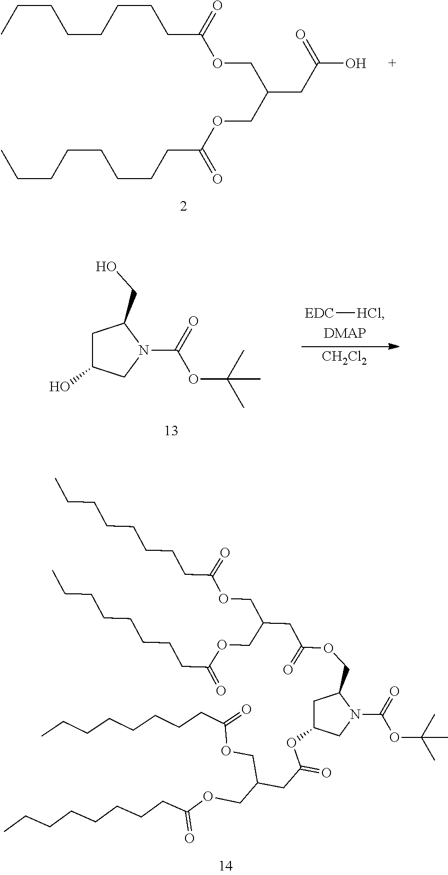

To a solution of 13 (3.10 g, 14.27 mmol, TCl America #B3662) in CH₃CN (60 mL), at room temperature under nitrogen, was added in order: 2 (12.0 g, 28.94 mmol), DMAP (1.80 g, 14.73 mmol), and EDC-HCl (7.00 g, 36.70 mmol). The mixture was stirred for 18 hours at room temperature, then was concentrated in vacuo to afford crude 14. Crude 14 was dissolved in n-heptane (60 mL), washed with MeOH/10% aq. citric acid (5:1, 2×60 mL), and water (60 mL). The organic phase was concentrated in vacuo and the residue was dissolved in CH₂Cl₂ (20 mL). To the solution of crude 14 was added silica gel (25g, type ZCX-2, 200-300 mesh) and the solvent was removed in vacuo to furnish silica gel impregnated with crude 14. This silica gel was placed atop a combi-flash column of silica gel (210g, type ZCX-2, 200-300 mesh) and the column was eluted with a gradient of CH₂Cl₂/MeOH (100:0 to 90:10). Qualified fractions were combined and concentrated in vacuo to yield 14 (5.80 g, 5.82 mmol, 41%) as a viscous, pale-yellow oil.

¹H-NMR (300 MHz, CDCl₃): δ=5.28 (brm, 1H), 4.04-4.37 (8H), 3.61 (brm, 1H), 2.55 (m, 2H), 2.42 (m, 4H), 2.32 (t, J=7.5 Hz, 8H), 2.18 (brm, 2H), 1.60-1.75 (10H), 1.5 (9H), 1.22-1.38 (40H), 0.88 (m, 12H); LCMS: RT 2.017, Calcd. for C₅₅H₉₇NO₁₄+Na+ 1032.70. Found 1032.60.

### Example 15: Synthesis of (2S,4R)-4-((4-(nonanoyloxy)-3-((nonanoyloxy)methyl)butanoyl)oxy)-2-(((4-(nonanoyloxy)-3-((nonanoyloxy)methyl)butanoyl)oxy)methyl)pyrrolidin-1-ium trifluoroacetate (15)

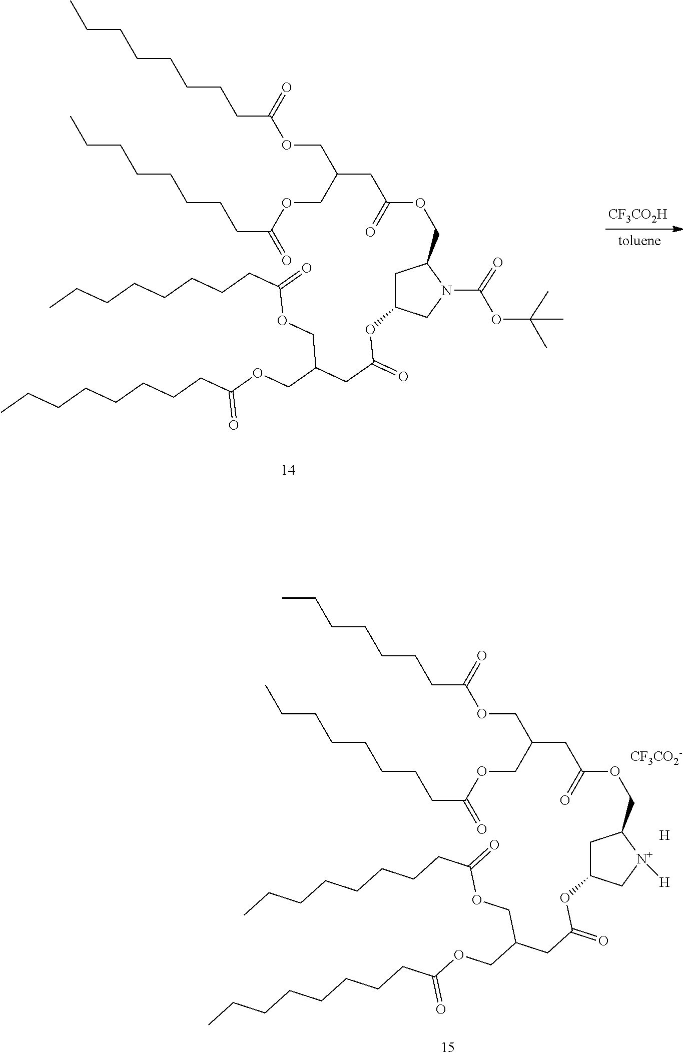

To a solution of 14 (5.80 g, 5.74 mmol) in toluene (36 mL), cooled to 20° C. under nitrogen, was added TFA (12 mL, 17.87 g, 157 mmol) over a period of 15 minutes. After the addition was complete, the mixture was stirred at 20° C. for 2 hours, then was concentrated in vacuo to give crude 15. Crude ammonium trifluoroacetate salt 15 was dissolved in n-heptane (60 mL) and the resulting solution was washed with 10% aq. K₂HPO₄ (30 mL) and water (2×60 mL). The organic layer was concentrated in vacuo to give 15 (5.50 g, 85% HPLC purity, 5.37 mmol, 94% yield) as a viscous, yellow oil.

¹H-NMR (300 MHz, CDCl₃): δ=5.50 (m, 1H), 4.62 (m, 1H), 4.26-4.36 (3H), 4.08-4.24 (8H), 3.89 (m, 1H), 3.72 (m, 1H), 2.61 (m, 2H), 2.25-2.49 (15H), 1.63 (m, 8H), 1.20-1.43 (40H), 0.90 (t, J=6.6 Hz, 12H); LCMS: RT 1.679, Calcd. for C₅₁H₉₂NO₁₂ 910.66, Found 910.60.

### Example 16: Synthesis of 2-(2-(((2S,4R)-1-(1H-imidazole-1-carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (16)

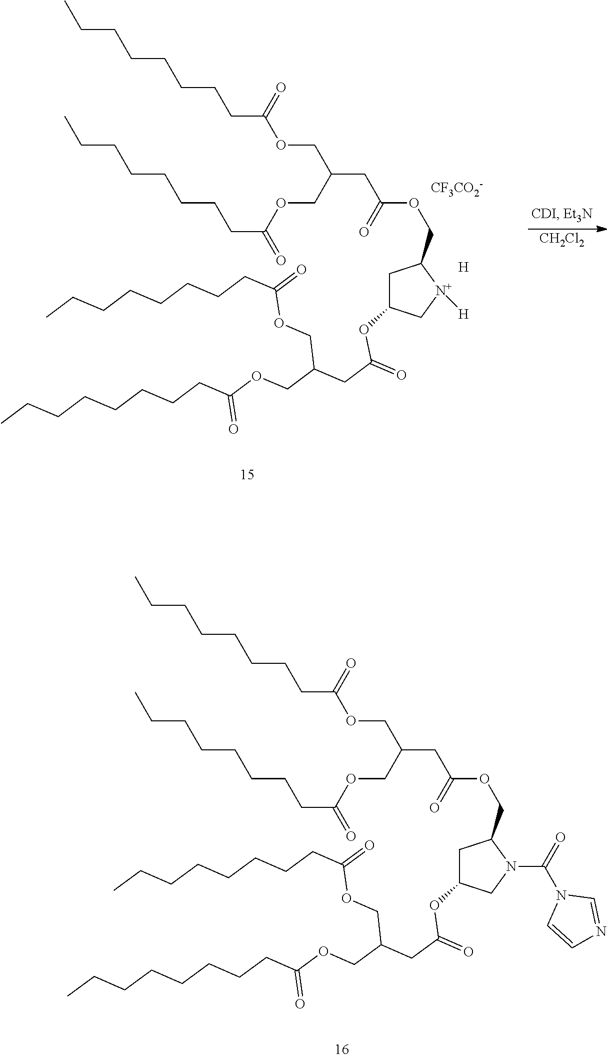

To a solution of 15 (5.50 g, 5.37 mmol) in CH₂Cl₂ (60 mL), cooled to 20° C. under nitrogen, was added in order CDI (3.48 g, 21.48 mmol) and Et₃N (1.09 g, 10.74 mmol). The solution was warmed to room temperature after the additions were complete, and the mixture was allowed to stir for 20 hours. The solution was cast into 1M aq. HCl (70 mL), the organic phase was separated. The aq. layer was extracted with CH₂Cl₂ (3×70 mL) and the combined organic phases were concentrated in vacuo to afford crude 16. Crude 16 was dissolved in n-heptane (150 mL) and the solution was washed with MeOH/water (2×150 mL), brine (150 mL), and dried (Na₂SO₄). Filtration and concentration in vacuo provided 16 (4.80 g, 4.78 mmol, 89% yield) as a pale-yellow oil.

¹H-NMR (300 MHz, CDCl₃): δ=8.75 (s, 1H), 7.56 (s, 1H), 7.32 (s, 1H), 5.37 (m, 1H), 4.80 (m, 1H), 4.68 (m, 2H), 3.99-4.28 (10H), 3.75 (d, J=12 Hz, 1H), 2.38-2.60 (4H), 2.25-2.37 (11H), 1.62 (m, 8H), 1.24-1.40 (40H), 0.90 (t, J=6.6 Hz, 12H); LCMS: RT 1.709, Calcd. for C₅₅H₉₃N₃O₁₃+H⁺ 1004.68. Found 1004.60.

### Example 17: Synthesis of 2-(2-(((2S,4R)-1-((2-(dimethylamino)ethoxy)carbonyl)-4-((4-(nonanoyloxy)-3((nonanoyloxy)methyl)butanoyl)oxy)pyrrolidin-2-yl)methoxy)-2-oxoethyl)propane-1,3-diyl dinonanoate (CICL-207)

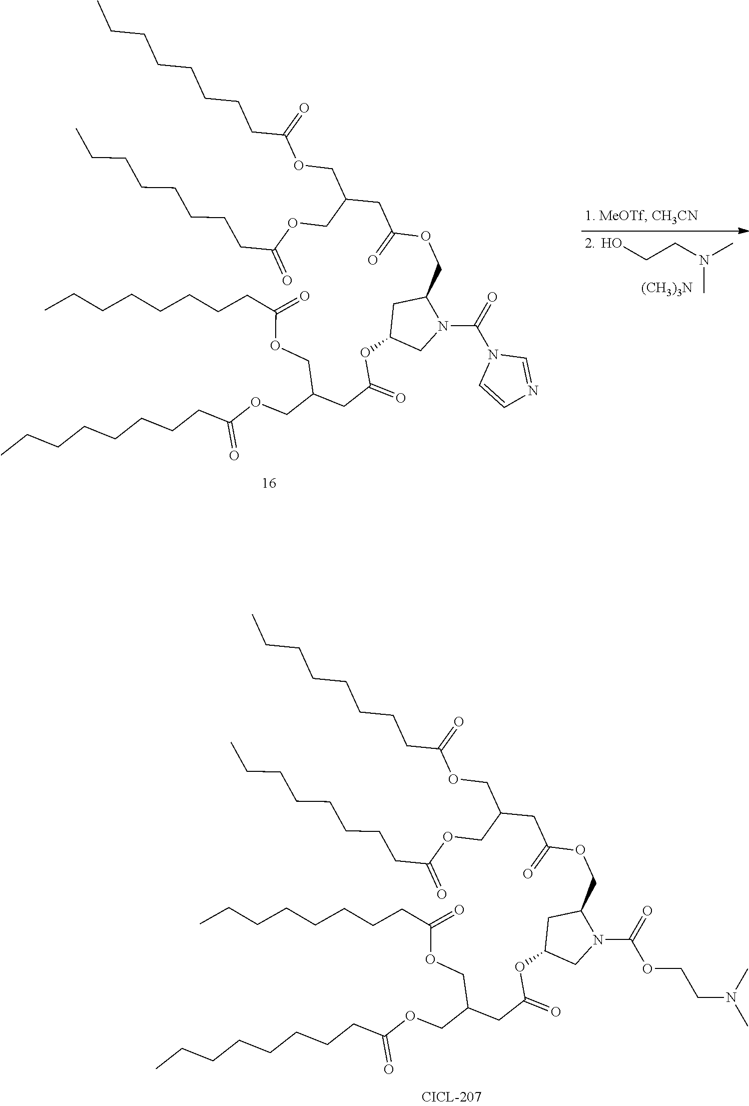

To a solution of 16 (4.80 g, 4.78 mmol), in CH3CN (50 mL), cooled in an ice-water bath under nitrogen was added MeOTf (0.862 g, 5.25 mmol) over a period of 5 minutes. The mixture was allowed to stir for 2 hours at 0° C., then a solution of trimethylamine in THF (2.0M, 7.20 mL, 14.40 mmol) was added over a period of 10 minutes followed by the addition of 2-dimethylaminoethanol (0.638 g, 7.16 mmol) in one portion. The mixture was stirred for 1 hour at 0° C., then, was warmed to 60° C. and stirred for 120 hours. The reaction mixture was concentrated in vacuo and the residue was dissolved in n-heptane (100 mL). The solution was washed with MeOH/water (5:1, 2×100 mL), MeOH/5.6% aq. citric acid (10:1). The organic phase was separated, the aq. Citric acid/MeOH phase was extracted with n-heptane (5×50 mL). To the MeOH/aq. citric acid phase was added n-heptane (200 mL) and 4.6% aq. Na₂CO₃ (100 mL), and the organic phase was separated, washed with MeOH/water (5:1, 100 mL). The organic phase was separated, dried over Na₂SO₄, and filtered. The filtrate was concentrated in vacuo to yield CICL-207 (4.00 g, 84.1% purity by HPLC) which was further purified by reverse phase prep-HPLC. CICL-207 was dissolved in CH₃CN (10 mL), and purified by chromatography (Xselect column CSH Phenyl-Hexyl 19×250 mm, 5 μM; Mobile Phase A: water/0.1% TFA; Mobile Phase B: CH₃CN; flow rate 20 mL/min; Gradient 65% B to 85% B in 10 minutes; Wave Length 200 nM; RT (min): 9.5). Qualified fractions were concentrated in vacuo, to remove CH₃CN, and the pH of the aq. Residue was adjusted to 7.0 with satd. Aq. Na₂CO₃. The aq. Phase was extracted with n-heptane (3×100 mL), and the combined organic phases were dried over Na₂SO₄. Filtration and concentration in vacuo gave CICL-207 (2.11 g, 2.06 mmol, 99% purity by HPLC, 43% yield) as a clear, colorless, viscous oil.

¹H-NMR (300 MHz, CDCl₃): δ=5.29 (m, 1H), 4.18-4.40 (5H), 4.11 (m, 8H), 3.60 (m, 2H), 2.48-2.70 (4H), 2.02-2.45 (20H), 1.61 (m, 8H), 1.19-1.39 (40H), 0.88 (m, 12H); LCMS: RT 1.127, Calcd. for C₅₆H₁₀₀N₂O₁₄+H⁺ 1025.73. Found 1025.80.

### Example 18: Synthesis of 2-(2-(((2S,4S)-1-(tert-butoxycarbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (18)

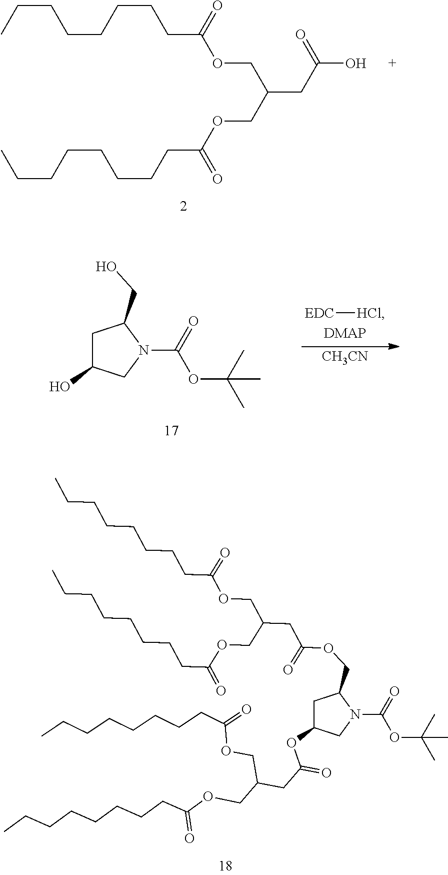

To a solution of 17 (2.00 g, 9.21 mmol, ACS Med. Chem. Lett. 2014, 5, 56) in CH₃CN (115 mL), at room temperature under nitrogen, was added in order: 2 (7.63 g, 18.42 mmol), DMAP (1.14 g, 9.33 mmol), and EDC-HCl (3.89 g, 20.40 mmol). The mixture was stirred for 18 hours at room temperature, then the reaction was quenched by the addition of water (115 mL). The resulting mixture was cast into n-heptane (200 mL) and the organic phase was separated. The organic layer was washed with MeOH/10% aq. Citric acid (75:25, 2×200 mL), MeOH/water/Et₃N (70:22:8, 2×100 mL), MeOH/water (75:25, 21×100 mL), brine (100 mL), and dried over Na₂SO₄. Filtration and concentration in vacuo gave 18 (6.65 g, 6.58 mmol, 93% HPLC purity, 71.5% yield) as a pale-yellow oil.

¹H-NMR (400 MHz, CDCl₃): δ=5.33 (m, 1H), 4.18-4.40 (2H), 4.02-4.18 (10H), 3.72 (m, 1H), 3.43 (m, 1H), 2.58 (m, 2H), 2.43 (m, 4H), 2.20-2.37 (10H), 1.62 (m, 8H), 1.50 (m, 8H), 1.22-1.40 (40H), 0.89 (t, J=6.8 Hz, 12H); LCMS: RT 1.997, Calcd. for C₅₆H₁₄NO₁₄+Na+ 1032.70. Found 1032.70.

### Example 19: Synthesis of (2S,4S)-4-((4-(nonanoyloxy)-3-((nonanoyloxy)methyl)butanoyl)oxy)-2-(((4-(nonanoyloxy)-3-((nonanoyloxy)methyl)butanoyl)oxy)methyl)pyrrolidin-1-ium trfluoroacetate (19)

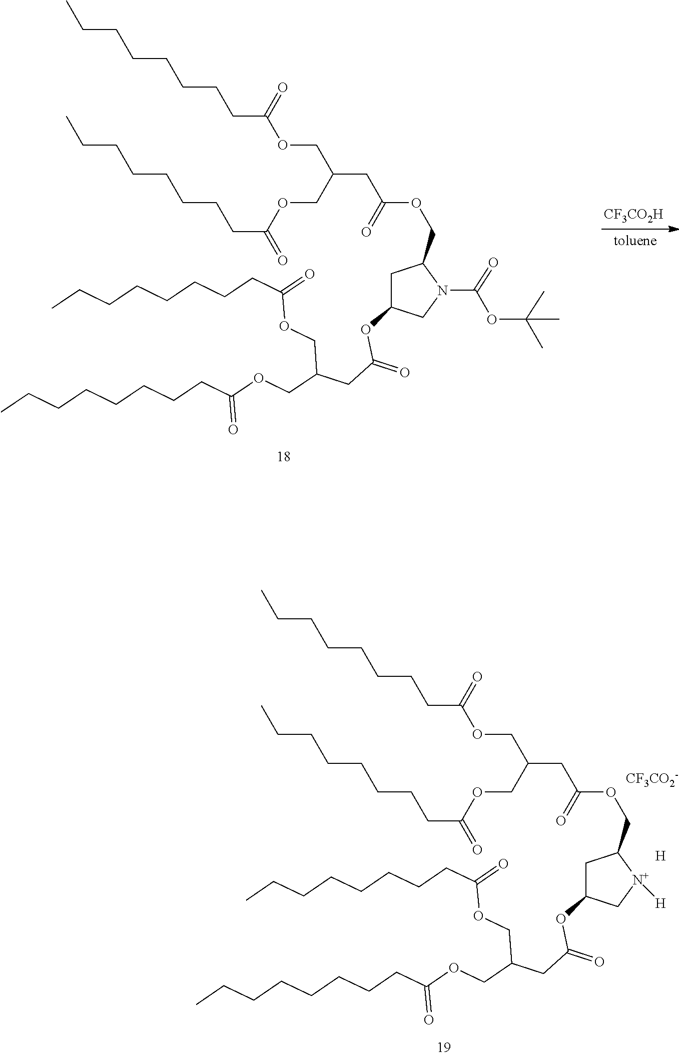

To a solution of 18 (6.50 g, 6.43 mmol) in toluene (65 mL), at room temperature under nitrogen, was added TFA (20 mL, 29.78 g, 261 mmol) over a period of 15 minutes. After the addition was complete, the mixture was stirred at room temperature for 18 hours, then was concentrated in vacuo to give crude 19. Crude ammonium trifluoroacetate salt 19 was dissolved in n-heptane (200 mL) and the resulting solution was washed with brine (2×200 mL) and dried over Na₂SO₄. Filtration and concentration in vacuo provided 19 (6.30 g, 93% HPLC purity, 6.24 mmol, 97% yield) as a viscous, yellow oil.

¹H-NMR (400 MHz, CDCl₃): δ=10.44 (brs, 1H), 9.98 (brs, 1H), 5.31 (m, 1H), 4.42 (m, 4H), 4.02-4.21 (8H), 3.60 (m, 2H), 2.37-2.60 (4H), 2.35 (m, 8H), 2.10 (m, 1H), 1.62 (m, 8H), 1.21-1.38 (40H), 0.89 (t, J=6.8 Hz, 12H); LCMS: RT 1.622, Calcd. for C₅₁H₉₂NO₁₂ 910.66. Found 910.60.

### Example 20: Synthesis of 2-(2-(((2S,4S)-1-(1H-Imidazole-1-carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (20)

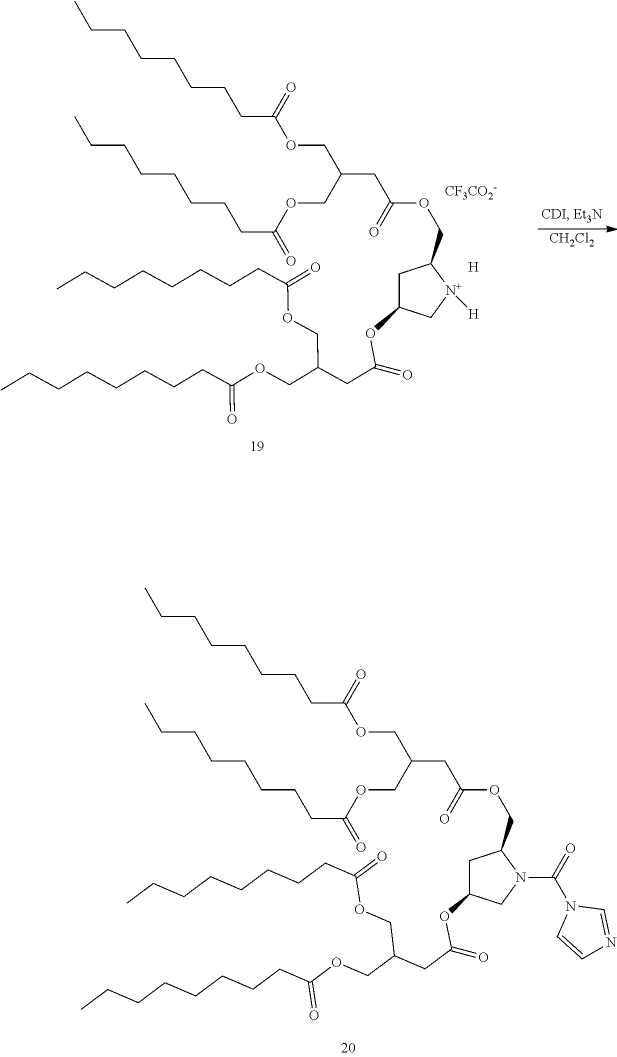

To a solution of 19 (6.20 g, 6.20 mmol) in CH₂Cl₂ (100 mL), at room temperature under GP-232,C³ nitrogen, was added CDI (9.96 g, 61.50 mmol) and Et₃N (3.11 g, 30.80 mmol). The resulting mixture was stirred for 18 hours at room temperature, then was cast into 0.5M aq. HCl (100 mL). The organic phase was separated, then was washed with 0.5M aq. HCl (100 mL), brine (2×100 mL), and dried over Na₂SO₄. Filtration and concentration in vacuo afforded crude 20 which was dissolved in CH₃CN and purified by reverse phase prep-HPLC (Column: XB Phenyl 50×250 mm, 10m; Mobile Phase A: water (0.1% CF₃CO₂H), Mobile Phase B: CH₃CN; Flow Rate: 90 mL/min; Gradient: 50% B to 85% B over 10 minutes, 85% collected; Wave Length: 220 nM). Qualified fractions were pooled, the acetonitrile was removed in vacuo and n-heptane (150 mL) was added to the resulting mixture. The organic phase was separated, washed with 5% aq. NaHCO₃ (200 mL) and dried (Na₂SO₄). Filtration and concentration in vacuo gave 20 (4.95 g, 4.93 mmol, 95% purity by HPLC, 80% yield) as a light-yellow oil.

¹H-NMR (400 MHz, CDCl₃): δ=8.07 (s, 1H), 7.36 (s, 1H), 7.12 (s, 1H), 5.31 (m, 1H), 4.72 (m, 1H), 4.33 (m, 2H), 4.22 (m, 1H), 4.08-4.18 (7H), 4.02 (m, 1H), 3.78 (m, 1H), 2.58 (m, 2H), 2.34-2.51 (5H), 2.26-2.31 (8H), 2.08 (m, 1H), 1.61 (m, 8H), 1.21-1.37 (40H), 0.89 (t, J=6.6 Hz, 12H); LCMS: RT 1.735, Calcd. for CH₅₅N₉₃N₃O₁₃+H⁺ 1004.68. Found 1004.60.

### Example 21: Synthesis of 2-(2-(((2S,4S)-1-((2-(dimethylamino)ethoxy)carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (CICL-224)

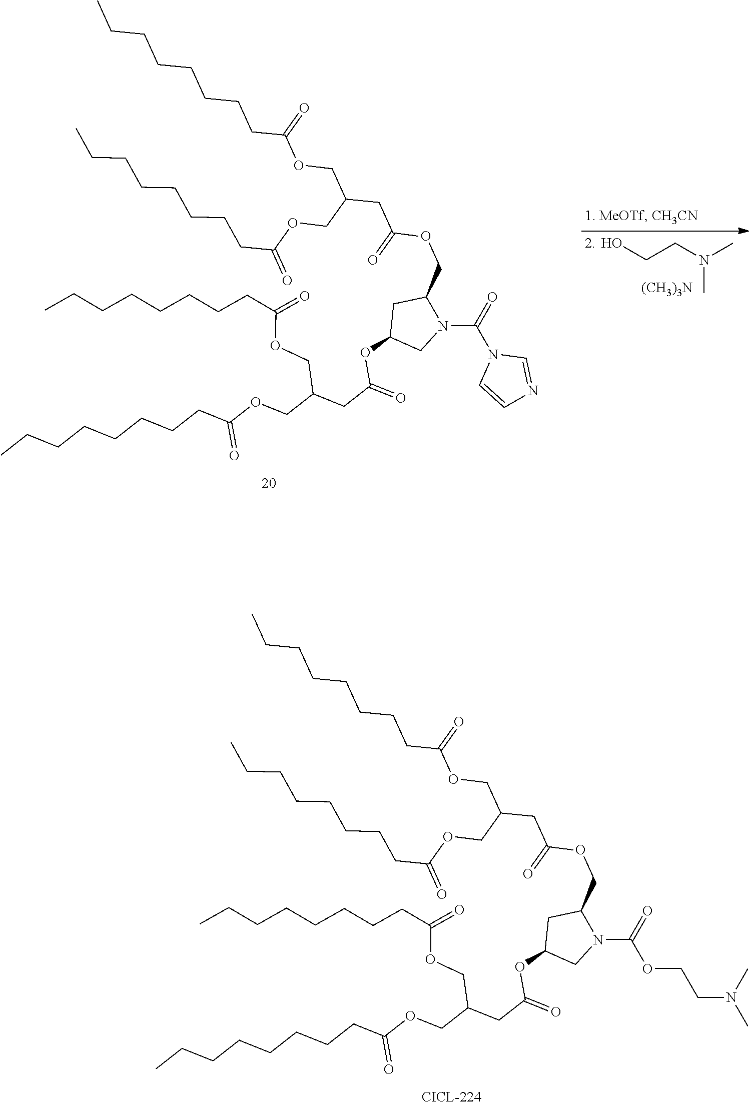

To a solution of 20 (4.00 g, 4.00 mmol), in CH₃CN (80 mL), cooled in an ice-water bath under nitrogen was added MeOTf (0.784 g, 4.80 mmol) over a period of 5 minutes. The mixture was allowed to stir for 2 hours at 0° C., then a solution of trimethylamine in THF (2.0M, 6.00 mL, 12.00 mmol) was added over a period of 10 minutes followed by the addition of 2-dimethylaminoethanol (0.532 g, 6.00 mmol) in one portion. The mixture was stirred for 1 hour at 0° C., then, was warmed to 60° C. and stirred for 72 hours. The reaction mixture was cast into n-heptane (160 mL) and water (160 mL). The organic phase was separated, washed with MeOH/water (4:1,3×100 mL), MeOH/5% aq. Citric acid (10:1). The organic phase was separated, the aq. citric acid/MeOH phase was diluted with 10% aq. NaCl (320 mL) and extracted with n-heptane (5×160 mL). The combined organic phases were washed with 5% aq. NaHCO₃ (2×100 mL), and the organic phase was separated and washed with water (2×100 mL). The organic phase was separated, dried over Na₂SO₄, and filtered. The filtrate was concentrated in vacuo and the residue was dissolved in CH₃CN (10 mL), and the crude product was purified by preparative reverse phase HPLC (XB Phenyl column CSH Phenyl-Hexyl 50×250 mm, 10 μM; Mobile Phase A: water/0.1% TFA; Mobile Phase B: CH₃CN; flow rate 90 mL/min; Gradient 60% B to 90% B in 10 minutes; Wave Length: 200 nM; RT (min): 12). Qualified fractions were concentrated in vacuo, to remove CH₃CN, and the pH of the aq. residue was adjusted to 9.0 with 5% aq. NaHCO₃. The aq. phase was extracted with n-heptane (200 mL), and the organic phase was washed with MeOH/water (5:1, 100 mL), water (100 mL) and dried (Na₂SO₄). Filtration and concentration in vacuo gave CICL-224 (1.50 g, 1.46 mmol, 97% purity by HPLC, 37% yield) as a clear, colorless, viscous oil.

¹H-NMR (400 MHz, CDCl₃): δ=5.32 (m, 1H), 4.03-4.38 (13H), 3.78 (brm, 1H), 3.48 (brd, J=12.8 Hz, 1H), 2.55 (m, 4H), 2.43 (m, 4H), 2.20-2.37 (14H), 2.07 (m, 2H), 1.61 (m, 8H), 1.21-1.36 (40H), 0.88 (m, 12H); LCMS: RT 1.588; Calcd. for C₅₈H₁₀₀N₂O₁₄+H⁺ 1025.73. Found 1025.60.

### Example 22: Synthesis of 2-(2-(((2R,4S)-1-(tert-butoxycarbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (22)

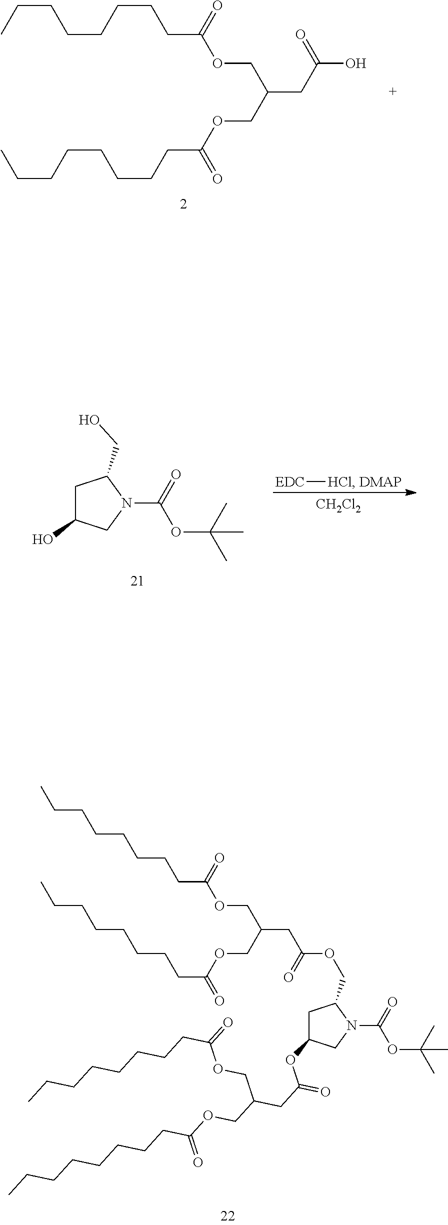

To a solution of 21 (2.20 g, 10.10 mmol, ACS Med. Chem. Lett. 2014, 5, 56) in CH₂Cl₂ (160 mL), at room temperature under nitrogen, was added in order: 2 (8.40 g, 20.30 mmol), DMAP (1.25 g, 10.10 mmol), and EDC-HCl (4.28 g, 22.30 mmol). The mixture was stirred for 18 hours, then was concentrated in vacuo. The residue was dissolved in n-heptane (500 mL), then the n-heptane solution was washed with MeOH/Water and dried over Na₂SO₄. (4:1, 3×150 mL). Filtration afforded a solution of crude 22 to which was added silica gel (25g, Type: ZCX-2, 100-200 mesh) and the solvent was then removed in vacuo (temperature <35° C.). The resulting 22 impregnated silica gel was placed atop a combi-flash column charged with silica gel (250g, Type: ZCX-2, 100-200 mesh), and the combi-flash column was eluted with a gradient of n-heptane/ethyl acetate (100:0 to 90:10). Qualified fractions were combined and concentrated in vacuo to furnish 22 (8.30 g, 8.21 mmol, HPLC purity 98.5%, 81% yield as a pale-yellow oil

¹H-NMR (400 MHz, CDCl₃): δ=5.26 (m, 1H), 4.00-4.30 (11H), 3.54 (m, 1H), 2.54 (m, 2H), 2.40 (m, 4H), 2.25-2.38 (7H), 2.17 (m, 2H), 1.63 (m, 8H), 1.50 (m, 8H), 1.21-1.38 (40H), 0.88 (m, 12H); LCMS: RT 2.478, Calcd. for C₅₆H₉₉NO₁₄+Na+ 1032.70. Found 1032.70.

### Example 23: Synthesis of (2R,4S)-4-((4-(nonanoyloxy)-3-((nonanoyloxy)methyl)butanoyl)oxy)-2-(((4-(nonanoyloxy)-3-((nonanoyloxy)methyl)butanoyl)oxy)methyl)pyrrolidin-1-ium trfluoroacetate (23)

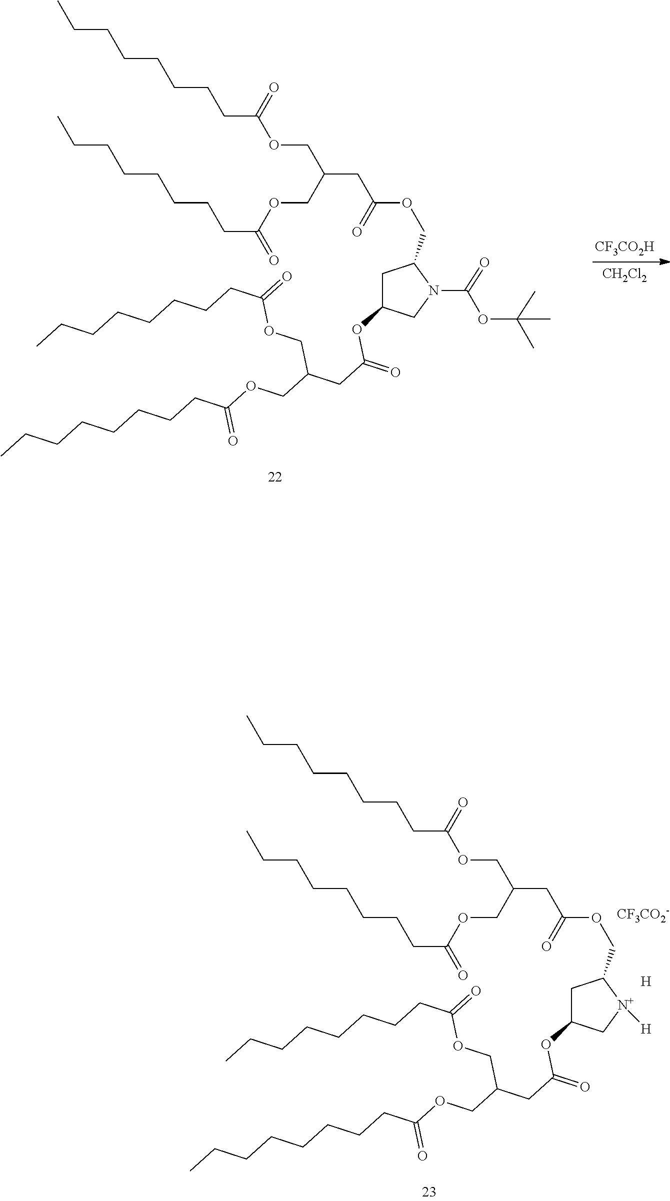

To a solution of 22 (8.30 g, 8.22 mmol) in dichloromethane (50 mL), at room temperature under nitrogen, was added TFA (16 mL, 23.82 g, 209 mmol) over a period of 15 minutes. After the addition was complete, the mixture was stirred at room temperature for 3 hours, then was concentrated in vacuo to give crude 23. Crude ammonium trifluoroacetate salt 23 was dissolved in n-heptane (400 mL) and the resulting solution was washed with brine (2×200 mL) and dried over Na₂SO₄. Filtration and concentration in vacuo provided 23 (8.10 g, 92% HPLC purity, 7.90 mmol, 96% yield) as a viscous, light-yellow oil.

¹H-NMR (400 MHz, CDCl₃): δ=10.44 (brs, 1H), 9.98 (brs, 1H), 5.40 (m, 1H), 4.65 (m, 1H), 4.37 (m, 1H), 4.13 (m, 8H), 3.78 (m, 1H), 3.49 (m, 1H), 2.40-2.62 (6H), 2.24-2.37 (10H), 1.61 (m, 8H), 1.21-1.34 (40H), 0.88 (m, 12H); LCMS: RT 1.612, Calcd. for C₅₁H₉₂NO₁₂ 910.66. Found 910.60.

### Example 24: Synthesis of 2-(2-(((2R,4S)-1-(1H-imidazole-1-carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (24)

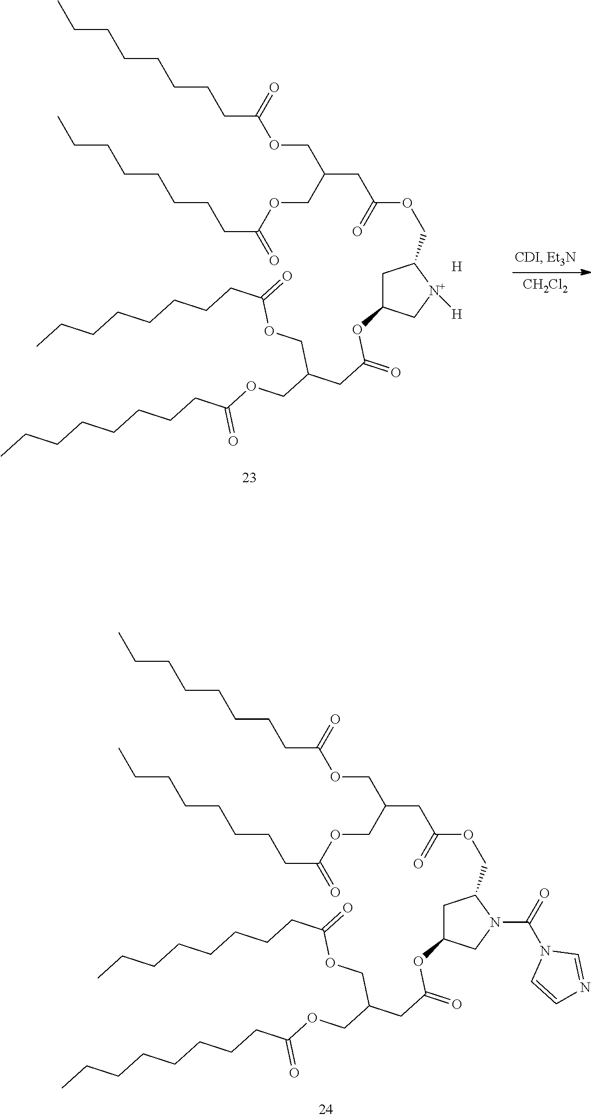

To a solution of 23 (8.10 g, 8.03 mmol) in CH₂Cl₂ (250 mL), at room temperature under nitrogen, was added CDI (13.03 g, 80.33 mmol) and Et₃N (4.06 g, 40.17 mmol). The resulting mixture was stirred for 18 hours at room temperature, then was cast into 0.5M aq. HCl (300 mL). The organic phase was separated, then was washed with 0.5M aq. HCl (300 mL), brine (2×300 mL), and dried over Na₂SO₄. Filtration and concentration in vacuo afforded crude 24 which was dissolved in CH₃CN (30 mL) and purified by reverse phase prep-HPLC (Column: XB Phenyl 50×250 mm, 10m; Mobile Phase A: water (0.1% CF₃CO₂H), Mobile Phase B: CH3CN; Flow Rate: 90 mL/min; Gradient: 50% B to 85% B over 10 minutes, 85% collected; Wave Length: 220 nM). Qualified fractions were pooled, the acetonitrile was removed in vacuo and n-heptane (150 mL) was added to the resulting mixture. The organic phase was separated, washed with 5% aq. NaHCO₃ (150 mL) and dried (Na₂SO₄). Filtration and concentration in vacuo gave 24 which was dissolved in n-heptane (400 mL) and the solution was extracted with MeOH/water (4:1, 150 mL), and dried over Na₂SO₄. Filtration and concentration in vacuo provided 24 (8.00 g, 7.96 mmol, 97.6% purity by HPLC, 99% yield) as a pale-yellow, viscous oil.

¹H-NMR (300 MHz, CDCl₃): δ=8.14 (s, 1H), 7.37 (s, 1H), 7.14 (s, 1H), 5.36 (m, 1H), 4.54-4.75 (2H), 4.01-4.27 (9H), 3.90 (m, 1H), 3.76 (m, 1H), 2.57 (m, 2H), 2.20-2.49 (14H), 1.61 (m, 8H), 1.22-1.38 (40H), 0.90 (t, J=7.20 Hz, 12H); LCMS: RT 1.878, Calcd. for C₅₅H₉₃N₃O₁₃+H⁺ 1004.68. Found 1004.60.

### Example 25: Synthesis of 2-(2-(((2R,4S)-1-((2-(dimethylamino)ethoxy)carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (CICL-225)

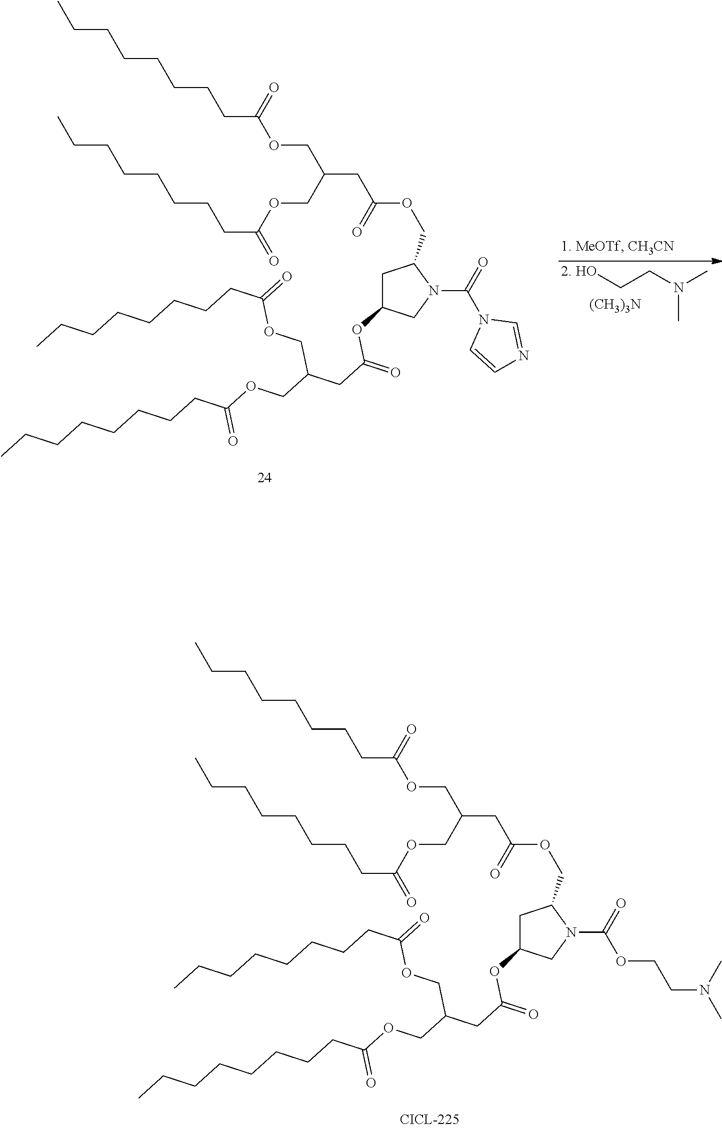

To a solution of 24 (8.00 g, 7.97 mmol), in CH₃CN (80 mL), cooled in an ice-water bath under nitrogen was added MeOTf (1.57 g, 9.57 mmol) over a period of 5 minutes. The mixture was allowed to stir for 2 hours at 0° C., then a solution of trimethylamine in THF (2.0M, 12.00 mL, 24.00 mmol) was added over a period of 10 minutes followed by the addition of 2-dimethylaminoethanol (1.06 g, 11.91 mmol) in one portion. The mixture was stirred for 1 hour at 0° C., then, was warmed to 60° C. and stirred for 120 hours. The reaction mixture was cast into n-heptane (400 mL) and washed with MeOH/water (5:1, 2×150 mL), the organic phase was then dried over Na₂SO₄, and filtered. The filtrate was concentrated in vacuo and the residue was dissolved in CH₃CN (15 mL), and the crude product was purified by preparative reverse phase HPLC (XB Phenyl column, 50×250 mm, 10 μM; Mobile Phase A: water/0.1% TFA; Mobile Phase B: CH₃CN; flow rate 90 mL/min; Gradient 60% B to 90% B in 10 minutes; Wave Length: 200 nM; RT (min): 12 Qualified fractions were concentrated in vacuo, to remove CH₃CN, and the pH of the aq. residue was adjusted to 9.0 with 5% aq. NaHCO₃. The aq. phase was extracted with n-heptane (300 mL), and the organic phase was washed with MeOH/water (5:1, 200 mL), water (200 mL) and dried (Na₂SO₄). Filtration and concentration in vacuo gave CICL-225 (2.017 g, 1.97 mmol, 97% purity by HPLC, 25% yield) as a clear, pale-yellow, viscous oil.

¹H-NMR (400 MHz, CDCl₃): δ=5.30 (brm, 1H), 4.19-4.40 (5H), 4.12 (m, 8H), 3.62 (brm, 2H), 2.50-2.68 (4H), 2.42 (m, 4H), 2.27-2.38 (14H), 2.21 (brm, 2H), 1.63 (m, 8H), 1.20-1.38 (40H), 0.90 (t, J=6.9 Hz, 12H); LCMS: Calcd. for C₅₈H₁₀₀N₂O₁₄+H⁺ 1025.73. Found 1025.60.

### Example 26: Synthesis of 2-(2-(((2R,4R)-1-(tert-butoxycarbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (26)

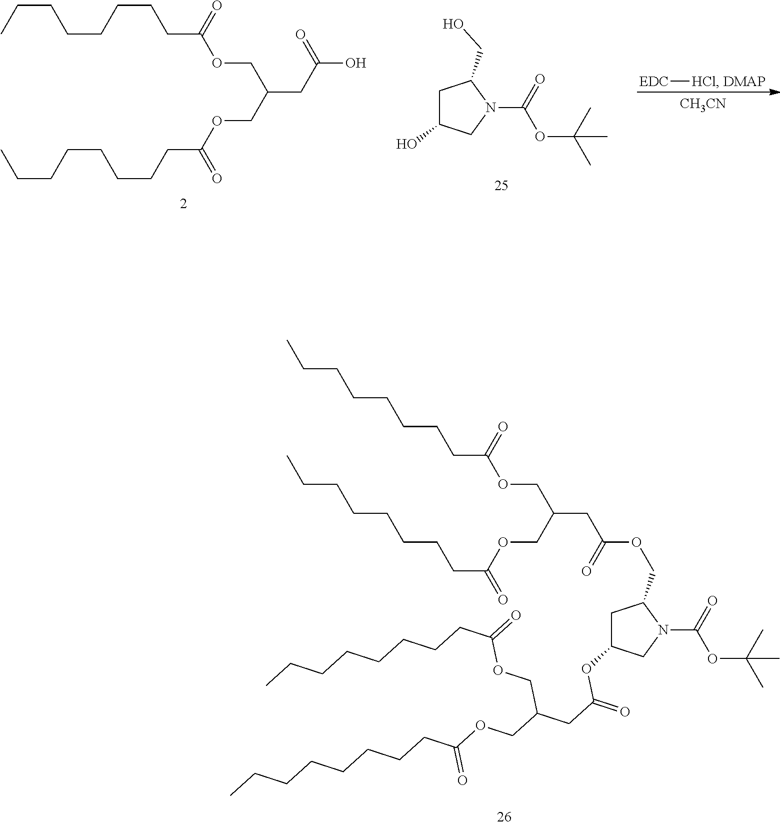

To a solution of 25 (1.83 g, 8.42 mmol, ACS Med. Chem. Lett. 2014, 5, 56) in CH₃CN (700 mL), at room temperature under nitrogen, was added in order: 2 (7.00 g, 16.88 mmol), DMAP (1.04 g, 8.51 mmol), and EDC-HCl (3.56 g, 18.67 mmol). The mixture was stirred for 18 hours, then the reaction was quenched by the addition of water (140 mL). The mixture was extracted with n-heptane (140 mL), and the organic phase was separated. The organic layer was washed with MeOH/10% aq. citric acid (3:1, 2×70 mL), brine (70 mL) and dried over Na₂SO₄. Filtration and concentration in vacuo provided 26 (5.65 g, 5.59 mmol, 92.7% purity by HPLC, 66% yield, as a pale-yellow oil.

¹H-NMR (400 MHz, CDCl₃): δ=5.33 (m, 1H), 4.30 (m, 1H), 4.06-4.21 (12H), 3.75 (m, 1H), 3.41 (m, 1H), 2.62 (m, 2H), 2.43 (m, 4H), 2.24 (m, 1H), 2.32 (m, 8H), 1.62 (m, 8H), 1.51 (m, 8H), 1.25-1.40 (40H), 0.89 (m, 12H); LCMS: RT 1.608, Calcd. for C₅₆H₉₉NO₁₄+Na+ 1032.70. Found 1032.70.

### Example 27: Synthesis of (2R,4R)-4-((4-(nonanoyloxy)-3-((nonanoyloxy)methyl)butanoyl)oxy)-2-(((4-(nonanoyloxy)-3-((nonanoyloxy)methyl)butanoyl)oxy)methyl)pyrrolidin-1-ium trfluoroacetate (27)

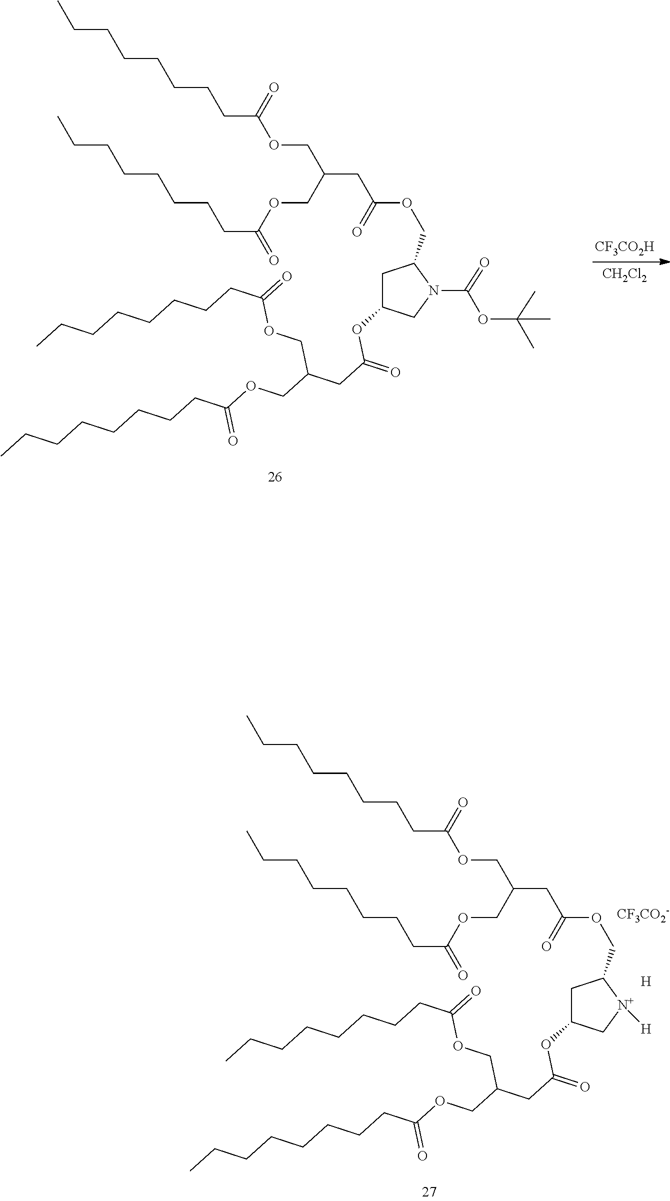

To a solution of 26 (5.60 g, 5.54 mmol) in dichloromethane (35 mL), at room temperature under nitrogen, was added TFA (11 mL, 16.38 g, 144 mmol) over a period of 15 minutes. After the addition was complete, the mixture was stirred at room temperature for 18 hours, then was concentrated in vacuo to give crude 27. Crude ammonium trifluoroacetate salt 23 was dissolved in n-heptane (200 mL) and the resulting solution was washed with brine (2×100 mL) and dried over Na₂SO₄. Filtration and concentration in vacuo provided 27 (5.55 g, 88% HPLC purity, 5.42 mmol, 98% yield) as a viscous, light-yellow oil.

¹H-NMR (400 MHz, CDCl₃): δ=10.21 (brs, 1H), 9.28 (brs, 1H), 5.45 (m, 1H), 4.38-4.52 (2H), 4.08-4.25 (9H), 3.50 (brs, 2H), 2.70 (m, 1H), 2.58 (m, 2H), 2.28-2.45 (12H), 2.14 (m, 1H), 1.63 (m, 8H), 1.22-1.37 (40H), 0.90 (m, 12H); LCMS: RT 1.602, Calcd. for C₅₁H₉₂NO₁₂ 910.66. Found 910.60.

### Example 28: Synthesis of 2-(2-(((2R,4R)-1-(1H-Imidazole-1-carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (28)

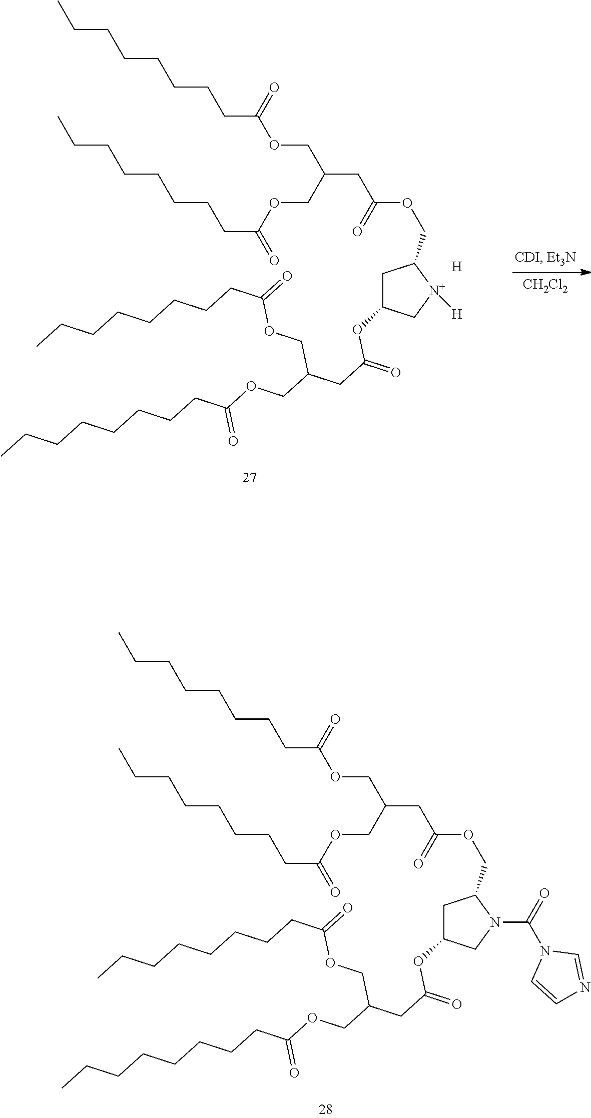

To a solution of 27 (5.55 g, 5.42 mmol) in CH₂Cl₂ (55 mL), at room temperature under nitrogen, was added CDI (8.92 g, 55.01 mmol) and Et3N (1.67 g, 16.50 mmol). The resulting mixture was stirred for 2 hours at room temperature, then was cast into 0.5M aq. HCl (40 mL). The organic phase was separated, then was washed with 0.5M aq. HCl (2×40 mL), water (3×20 mL), and dried over Na₂SO₄. Filtration and concentration in vacuo afforded crude 28 which was dissolved in CH₃CN (30 mL) and purified by reverse phase prep-HPLC (Column: XB Phenyl 50×250 mm, 10m; Mobile Phase A: water (0.1% CF₃CO₂H), Mobile Phase B: CH₃CN; Flow Rate: 90 mL/min; Gradient: 50% B to 85% B over 10 minutes, 85% collected; Wave Length: 220 nM). Qualified fractions were pooled, the acetonitrile was removed in vacuo and n-heptane (150 mL) was added to the resulting mixture. The organic phase was separated, washed with 5% aq. NaHCO₃ (150 mL), water (150 mL), and dried (Na₂SO₄). Filtration and concentration in vacuo gave 28 (3.75 g, 3.73 mmol, 96.5% purity by HPLC, 69% yield) as a pale-yellow, viscous oil.

¹H-NMR (300 MHz, CDCl₃): δ=8.07 (s, 1H), 7.36 (s, 1H), 7.12 (s, 1H), 5.31 (m, 1H), 4.72 (m, 1H), 4.33 (m, 2H), 4.22 (m, 1H), 4.08-4.18 (7H), 4.02 (m, 1H), 3.78 (m, 1H), 2.58 (m, 2H), 2.34-2.51 (5H), 2.26-2.31 (8H), 2.08 (m, 1H), 1.61 (m, 8H), 1.21-1.37 (40H), 0.89 (t, J=6.6 Hz, 12H); LCMS: RT 1.868, Calcd. for C₅₅N₉₃N₃O₁₃+H⁺ 1004.68. Found 1004.60.

### Example 29: Synthesis of 2-(2-(((2R,4R)-1-((2-(dimethylamino)ethoxy)carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (CICL-223)

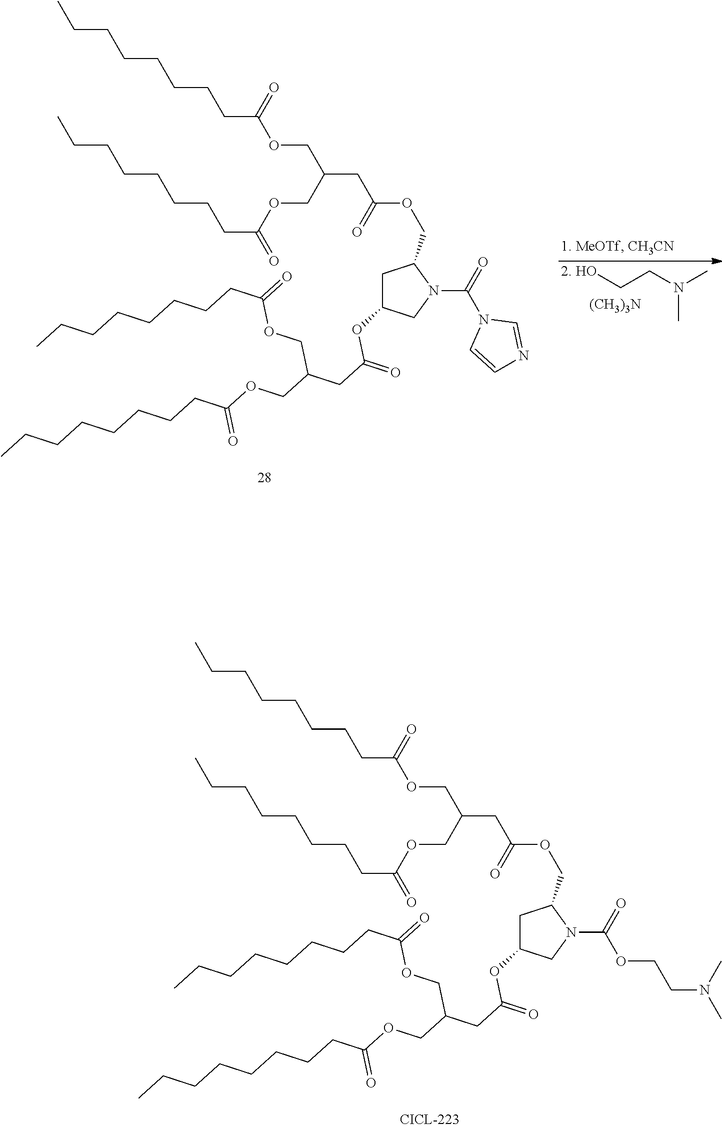

To a solution of 28 (3.75 g, 3.73 mmol), in CH₃CN (75 mL), cooled in an ice-water bath under nitrogen was added MeOTf (0.73 g, 4.45 mmol) over a period of 5 minutes. The mixture was allowed to stir for 2 hours at 0° C., then a solution of trimethylamine in THF (2.0M, 5.60 mL, 11.2 mmol) was added over a period of 10 minutes followed by the addition of 2-dimethylaminoethanol (0.50 g, 5.61 mmol) in one portion. The mixture was stirred for 1 hour at 0° C., then, was warmed to 60° C. and stirred for 100 hours. The reaction mixture was cast into n-heptane (200 mL) and washed with 10% aq. Citric acid (100 mL), water (300 mL), brine (300 mL), the organic phase was then dried over Na₂SO₄, and filtered. The filtrate was concentrated in vacuo and the residue was dissolved in i-PrOH (5 mL), and the crude product was purified by preparative reverse phase HPLC (XB Phenyl column, 50×250 mm, 10 μM; Mobile Phase A: water/0.1% TFA; Mobile Phase B: CH3CN; flow rate 90 mL/min; Gradient 55% B to 95% B in 32 minutes; Wave Length: 205 nM; RT (min): 17). Qualified fractions were concentrated in vacuo, to remove CH₃CN, and the pH of the aq. residue was adjusted to 9.0 with 2% aq. Na₂CO₃. The aq. phase was extracted with n-heptane (3×300 mL), and the organic phases were washed with MeOH/water (5:1, 2×300 mL), water (300 mL) and dried (Na₂SO₄). Filtration and concentration in vacuo gave CICL-223 (1.38 g, 1.35 mmol, 98% purity by HPLC, 36% yield) as a clear, pale-yellow, viscous oil.

¹H-NMR (300 MHz, CDCl₃): δ=5.33 (brm, 1H), 4.03-4.40 (13H), 3.79 (brm, 1H), 3.50 (brd, J=13.2 Hz, 1H), 2.52-2.68 (4H), 2.46 (m, 4H), 2.20-2.34 (14H), 2.07 (brm, 2H), 2.63 (m, 8H), 1.21-1.40 (40H), 0.90 (m, 12H); LCMS: RT 0.905, Calcd. for C₅₈H₁₀₀N₂O₁₄+H⁺ 1025.73. Found 1025.60.

### Example 30: Synthesis of 2-(2-(((2S,4R)-1-(((2-(dimethylamino)ethyl)thio)carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (CICL-217)

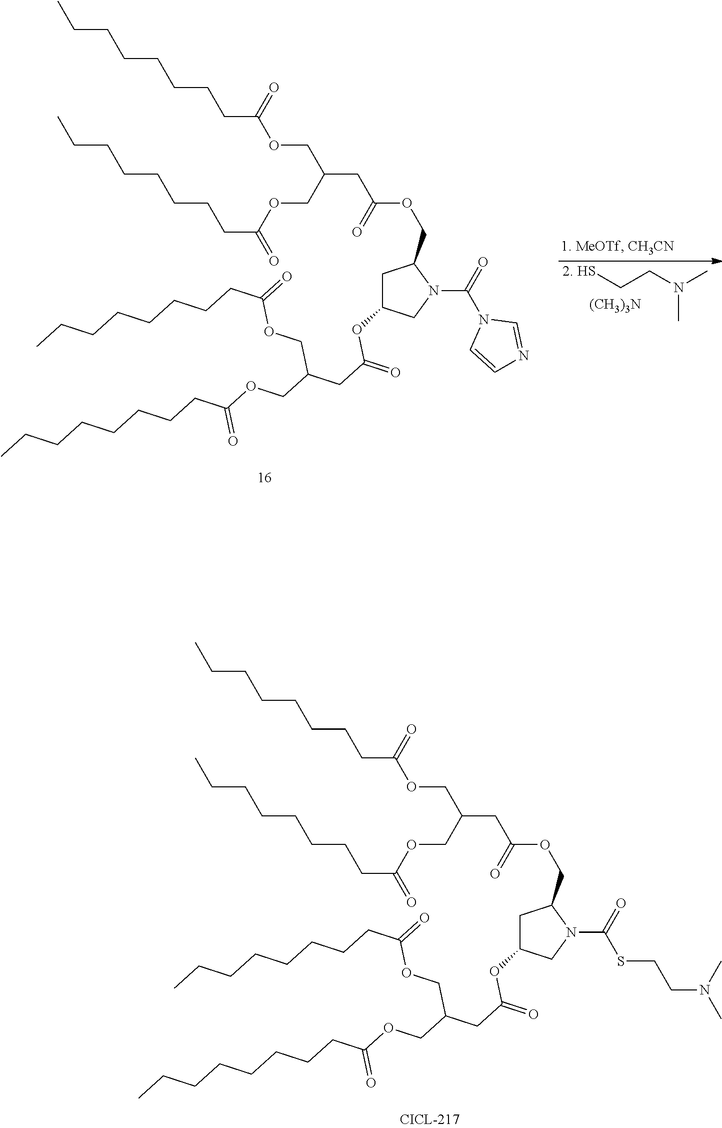

To a solution of 16 (7.00 g, 6.83 mmol), in CH₃CN (70 mL), cooled in an ice-water bath under nitrogen was added MeOTf (1.37 g, 8.35 mmol) over a period of 5 minutes. The mixture was allowed to stir for 2 hours at 0° C., then a solution of trimethylamine in THF (2.0M, 10.50 mL, 21.00 mmol) was added over a period of 10 minutes followed by the addition of 2-dimethylaminoethane thiol (1.10 g, 10.46 mmol) in one portion. The mixture was stirred for 1 hour at 0° C., then, was warmed to room temperature and was stirred for 18 hours. The reaction mixture concentrated in vacuo, the residue was dissolved with n-heptane (400 mL) and the resulting solution was washed with MeOH/water (5:1, 2×150 mL), the organic phase was then dried over Na₂SO₄, and filtered. The filtrate was concentrated in vacuo and the residue was dissolved in CH₃CN (15 mL), and the crude product was purified by preparative reverse phase HPLC (XB Phenyl column, 50×250 mm, 10 μM; Mobile Phase A: water/0.1% TFA; Mobile Phase B: CH₃CN; flow rate 90 mL/min; Gradient 50% B to 90% B in 18 minutes; Wave Length: 200 nM). Qualified fractions were concentrated in vacuo, to remove CH₃CN, and the pH of the aq. residue was adjusted to 8.0 with 2% aq. Na₂CO₃. The aq. phase was extracted with n-heptane (300 mL), and the organic phase was washed with MeOH/water (5:1, 200 mL), water (200 mL) and dried (Na₂SO₄). Filtration and concentration in vacuo gave CICL-217 (2.11 g, 2.03 mmol, 98% purity by HPLC, 30% yield) as a clear, pale-yellow, viscous oil.

¹H-NMR (300 MHz, CDCl₃): δ=5.35 (m, 1H), 4.42 (m, 2H), 4.26 (m, 1H), 4.11 (m, 8H), 3.66 (brm, 2H), 3.13 (m, 2H), 2.49-2.71 (4H), 2.18-2.48 (20H), 1.63 (m, 8H), 1.20-1.39 (40H), 0.88 (t, J=6.9 Hz, 12H); LCMS: RT 1.638, Calcd. for C₅₆H₁₀₀N₂O₁₃S+H+1041.70. Found 1041.60.

### Example 31: Synthesis of (((re-(3S,4R)-1-(tert-butoxycarbonyl)pyrrolidine-3,4-diyl)bis(oxy))bis(2-oxoethane-2,1-diyl))bis(propane-2,1,3-triyl) tetranonanoate (30)

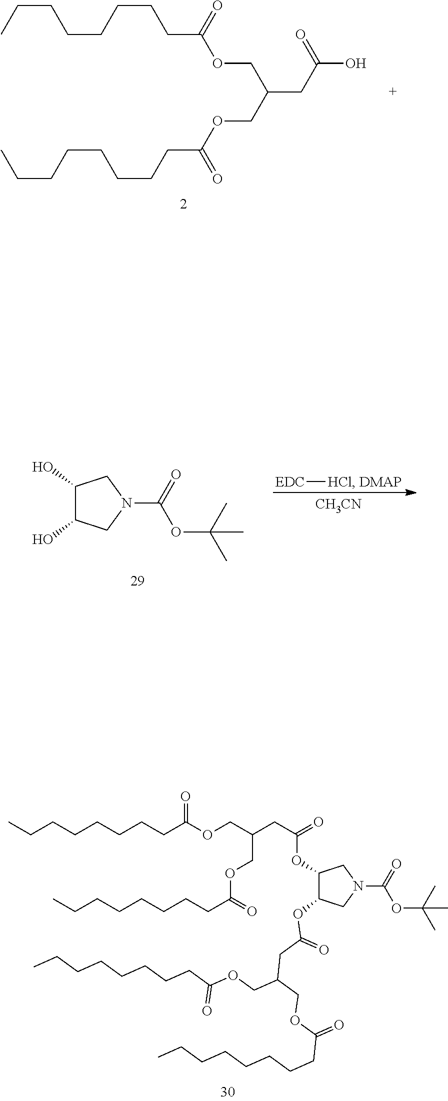

To a solution of 29 (Nature Communications 2019, 10, 21, 2.20 g, 10.84 mmol), in CH₃CN (44 mL) under nitrogen at room temperature, was added in order 2 (9.20 g, 22.22 mmol), DMAP (1.32 g, 10.84 mmol), and EDC-HCl (5.20 g, 27.09 mmol). The mixture was stirred for 18 hours at room temperature after the additions were complete, then the solvent was removed in vacuo. The residue was dissolved in n-heptane (440 mL) and the solution was washed with MeOH/water (8:1, 2×110 mL). The solvent was removed in vacuo to provide crude 30 which was purified by reverse phase prep-HPLC (Column: XB Phenyl 50×250 mm, 10m; Mobile Phase A: water (0.1% CF₃CO₂H), Mobile Phase B: CH₃CN; Flow Rate: 90 mL/min; Gradient: 50% B to 95% B over 10 minutes, 85% collected; Wave Length: 205 nM). Qualified fractions were pooled, the acetonitrile was removed in vacuo and the resulting solution was extracted with n-heptane (440 mL). The organic phase was washed with MeOH/water (4:1, 2×110 mL), water (110 mL), and dried (Na₂SO₄). Filtration and concentration in vacuo gave 30 (6.80 g, 6.82 mmol, HPLC purity 97%, 63% yield) as a viscous, colorless oil.

¹H-NMR (400 MHz, CDCl₃): δ=5.37 (brm, 2H), 4.13 (m, 8H), 3.73 (m, 2H), 3.28-3.51 (2H), 2.58 (m, 2H), 2.45 (m, 4H), 2.32 (t, J=6.0 Hz, 8H), 1.63 (m, 8H), 1.49 (s, 9H), 1.21-1.40 (40H), 0.90 (t, J=6.8 Hz, 12H); LCMS: RT 1.991, Calcd. for C₅₅H₉₇NO₁₄+Na+ 1018.68. Found 1018.60.

### Example 32: Synthesis of Rel-(3S,4R)-3,4-bis((4-(nonanoyloxy)-3-((nonanoyloxy)methyl)butanoyl)oxy)pyrrolidin-1-ium trfluoroacetate (31)

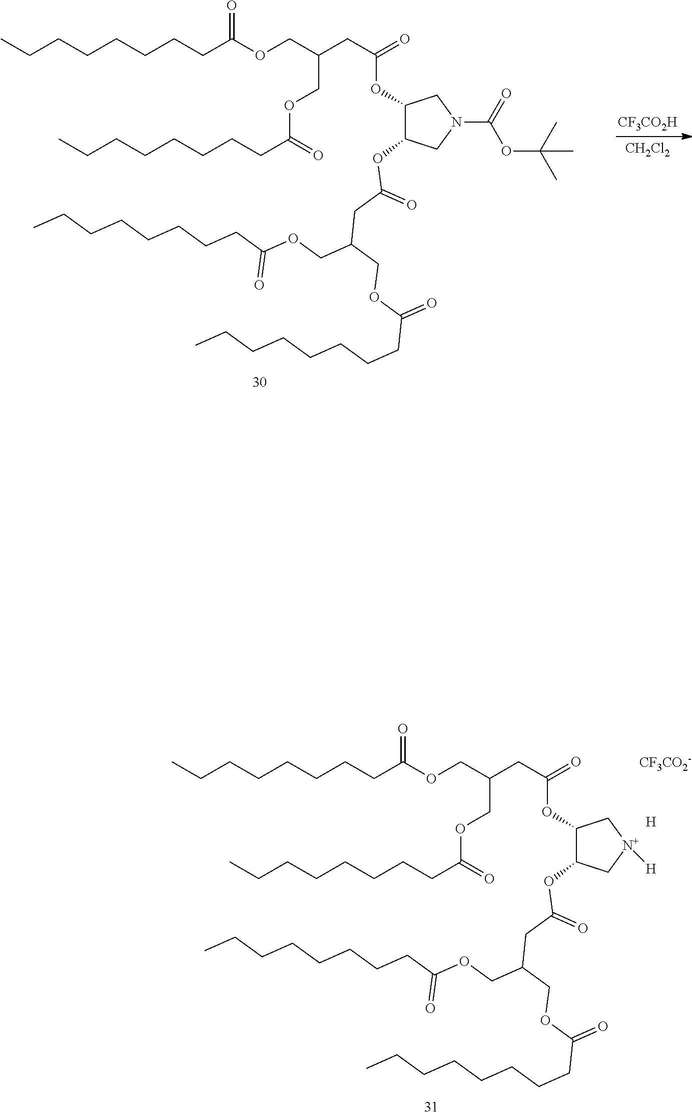

To a solution of 30 (6.60 g, 6.63 mmol), in CH₂Cl₂ (33 mL) under nitrogen at room temperature, was added CF₃CO₂H (13.2 mL, 19.65 g, 172 mmol) over a period of 10 minutes. The reaction mixture was allowed to stir for 2 hours after the addition was complete, and then was concentrated in vacuo. The residue was dissolved in n-heptane (660 mL) and the solution was extracted with brine (2×110 mL). The organic phase was concentrated in vacuo to afford 31 (6.20 g, 6.14 mmol, HPLC purity 97%, 93% yield) as a viscous, light-yellow oil.

¹H-NMR (400 MHz, CDCl₃): δ=5.48 (brm, 2H), 4.10-4.23 (8H), 3.72 (m, 2H), 3.49 (m, 2H), 2.56 (m, 2H), 2.47 (m, 4H), 2.32 (m, 8H), 1.63 (m, 8H), 1.23-1.37 (40H), 0.90 (t, J=6.8 Hz, 12H); LCMS: RT 1.601, Calcd. for C₅₀H₉₀NO₁₂ 896.65. Found 896.60.

### Example 33: Synthesis of (((Rel-(3S,4R)-1-(1H-Imidazole-1-carbonyl)pyrrolidine-3,4-diyl)bis(oxy))bis(2-oxoethane-2,1-diyl))bis(propane-2,1,3-triyl) tetranonanoate (32)

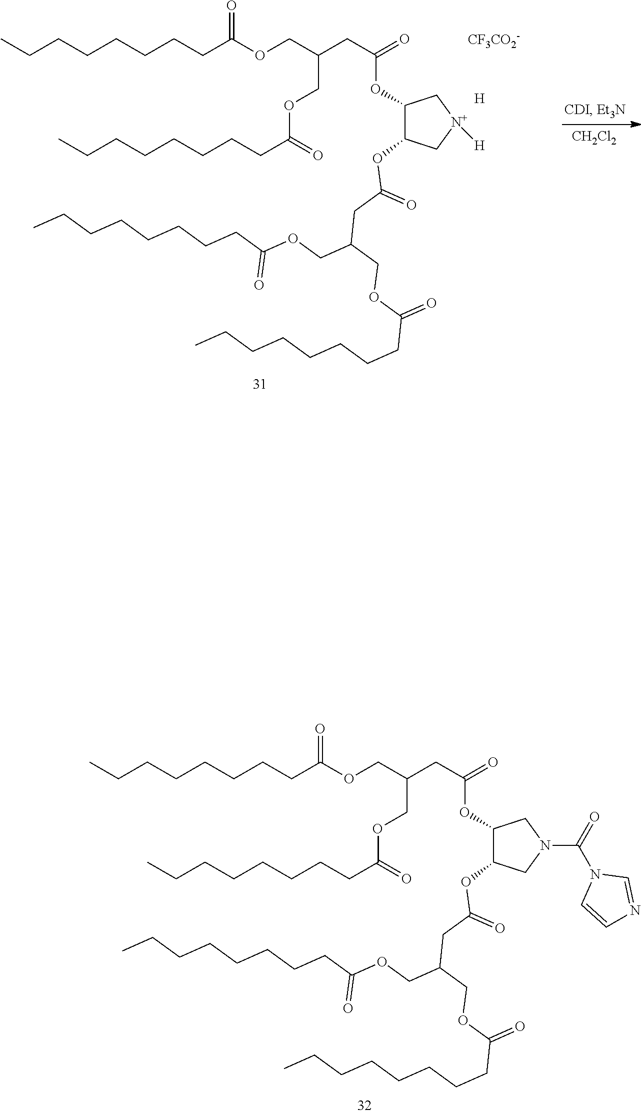

To a solution of 31 (6.00 g, 6.04 mmol) in dichloromethane (120 mL), at room temperature under nitrogen, was added in order: CDI (9.78 g, 60.36 mmol) and Et₃N (4.88 g, 48.29 mmol). The reaction mixture was stirred for 18 hours at room temperature, then was cast into 0.5M aq. HCl (200 mL). The organic phase was separated, the aq. phase was extracted with dichloromethane (2×240 mL) and the combined organic layers were concentrated in vacuo to yield crude 32. Crude 32 was dissolved in ne-heptane (600 mL) and the resulting solution was extracted with MeOH/water (5:1, 2×240 mL)) and water (240 mL). The solvent was removed in vacuo to afford 32 (5.60 g, 5.65 mmol, HPLC purity 96%, 94% yield) as a light-yellow oil.

¹H-NMR (400 MHz, CDCl₃): δ=8.27 (brm, 1H), 7.44 (brs, 1H), 7.18 (brs, 1H), 5.48 (brm, 2H), 3.92-4.23 (10H), 3.82 (brm, 2H), 2.57 (m, 2H), 2.46 (m, 4H), 2.32 (m, 8H), 1.63 (m, 8H), 1.23-1.37 (40H), 0.90 (t, J=6.8 Hz, 12H); LCMS: RT 1.760, Calcd. for C₅₄H₉₁N₃O₁₃+H⁺ 990.66. Found 990.50.

### Example 34: Synthesis of (((Rel-(3S,4R)-1-((2-(dimethylamino)ethoxy)carbonyl)pyrrolidine-3,4-diyl)bis(oxy))bis(2-oxoethane-2,1-diyl))bis(propane-2,1,3-triyl) tetranonanoate (CICL-216)

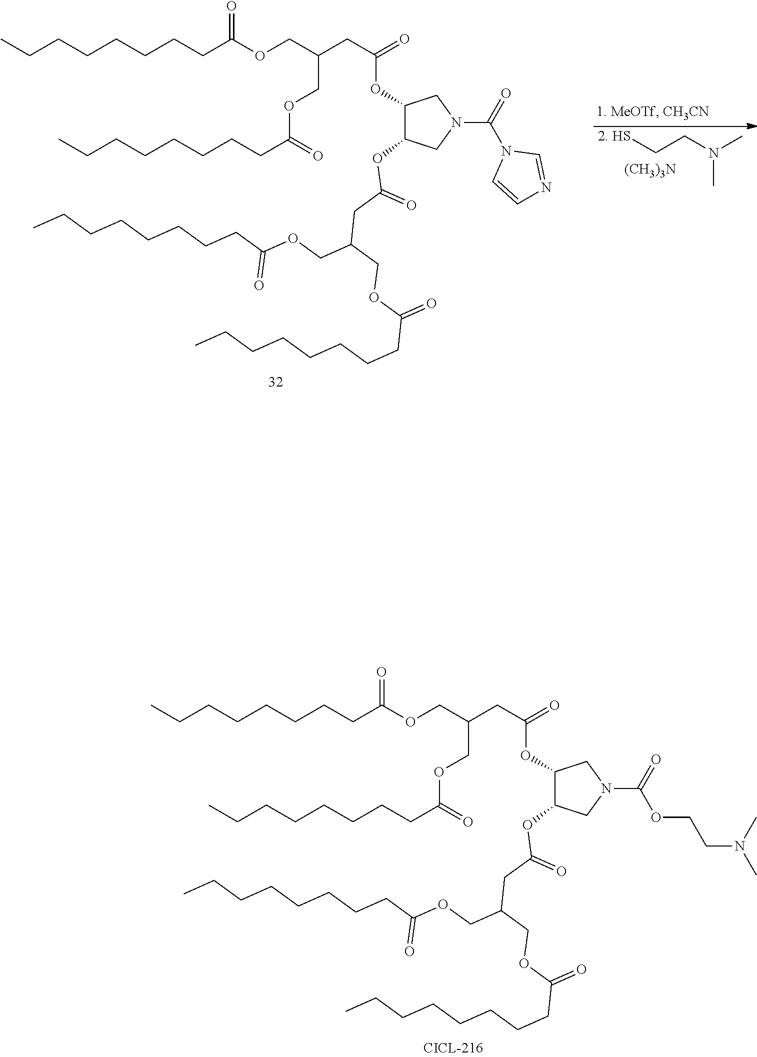

To a solution of 32 (5.50 g, 5.55 mmol), in CH₃CN (55 mL), cooled in an ice-water bath under nitrogen was added MeOTf (1.09 g, 6.66 mmol) over a period of 5 minutes. The mixture was allowed to stir for 2 hours at 0° C., then a solution of trimethylamine in THF (1.0M, 22.20 mL, 22.20 mmol) was added over a period of 10 minutes followed by the addition of 2-dimethylaminoethanol (0.743 g, 8.33 mmol) in one portion. The mixture was stirred for 1 hour at 0° C., then, was warmed in a 60° C. oil bath and was stirred for 120 hours. The reaction mixture concentrated in vacuo, the residue was dissolved with n-heptane (550 mL) and the resulting solution was washed with MeOH/water (5:1, 2×100 mL), acetonitrile/water (5:1, 2×100 mL), then the organic phase was then dried over Na₂SO₄, and filtered. The filtrate was concentrated in vacuo and the residue was dissolved in CH₃CN (15 mL), and the crude product was purified by preparative reverse phase HPLC (XB Phenyl column, 50×250 mm, 10 μM; Mobile Phase A: water/0.1% TFA; Mobile Phase B: CH3CN; flow rate 90 mL/min; Gradient 55% B to 90% B in 12 minutes; Wave Length: 205 nM). Qualified fractions were concentrated in vacuo, to remove CH₃CN, and the pH of the aq. residue was adjusted to 9.0 with 2% aq. Na₂CO₃. The aq. phase was extracted with n-heptane (2×275 mL), and the combined organic phases were washed with MeOH/water (4:1, 3×110 mL), water (145 mL) and dried (Na₂SO₄). Filtration and concentration in vacuo gave CICL-216 (2.22 g, 2.20 mmol, 98% purity by HPLC, 40% yield) as a clear, colorless, viscous oil.

¹H-NMR (400 MHz, CDCl₃): δ=5.37 (m, 2H), 4.20 (m, 2H), 4.11 (m, 8H), 3.78 (m, 2H), 3.48 (m, 2H), 2.50-2.65 (4H), 2.42 (m, 4H), 2.25-2.35 (14H), 1.59 (m, 8H), 1.20-1.38 (40H), 0.88 (m, 12H); LCMS: RT 1.575, Calcd. for C₅₅H₉₈N₂O₁₄+H+1011.71. Found 1011.70.

### Example 35: Synthesis of ((((3S,4S)-1-(tert-butoxycarbonyl)pyrrolidine-3,4-diyl)bis(oxy))bis(2-oxoethane-2,1-diyl))bis(propane-2,1,3-triyl) tetranonanoate (34)

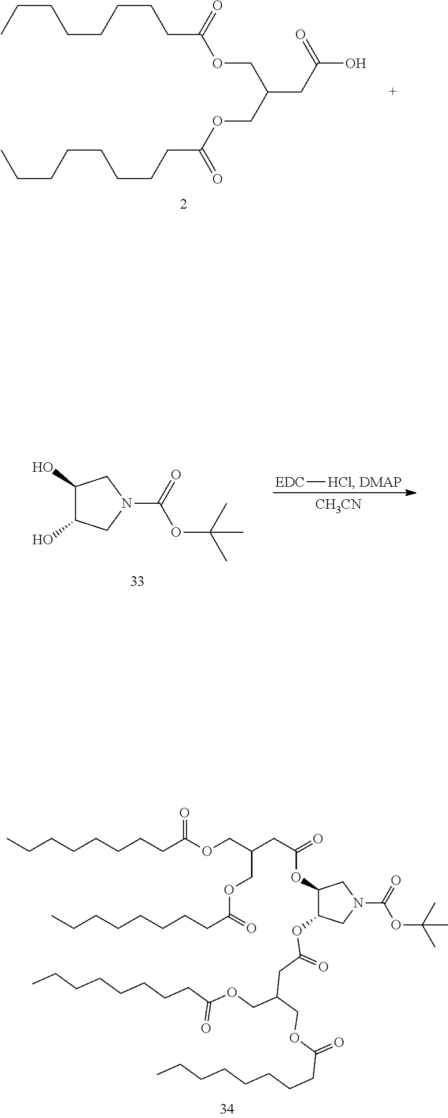

To a solution of 33 (Combi-Blocks #QE-0378, 2.35 g, 11.56 mmol) in acetonitrile (94 mL), at room temperature under nitrogen, was added in order 2 (10.00 g, 24.12 mmol), DMAP (1.42 g, 11.62 mmol), and EDC-HCl (5.55 g, 29.10 mmol). The reaction mixture was stirred for 16 hours at room temperature then was concentrated in vacuo. The residue was dissolved in n-heptane (200 mL) and the resulting solution was extracted with MeOH/water (10:1, 2×100 mL). The solvent was removed in vacuo and the residue was dissolved in dichloromethane (25 mL) and silica gel (25g, Type: ZCX-2, 100-200 mesh) was added. The solvent was then removed n vacuo (temperature <35° C.). The resulting 34 impregnated silica gel was placed atop a combi-flash column charged with silica gel (120g, Type: ZCX-2, 100-200 mesh), and the combi-flash column was eluted with a gradient of n-heptane/ethyl acetate (40:1 to 25:1). Qualified fractions were combined and concentrated in vacuo to furnish 34 (8.20 g, 8.23 mmol, H PLC purity 95%, 71% yield as a pale-yellow oil.

¹H-NMR (300 MHz, CDCl₃): δ=5.20 (brm, 2H), 4.13 (m, 8H), 3.73 (m, 2H), 3.40-3.62 (2H), 2.58 (m, 2H), 2.42 (m, 4H), 2.37 (m, 8H), 1.62 (m, 8H), 1.50 (s, 9H), 1.24-1.38 (40H), 0.90 (t, J=6.8 Hz, 12H); LCMS: RT 2.144, Calcd. for C₅₅H97NO₁₄+Na+ 1018.68. Found 1018.80.

### Example 36: Synthesis (3S,4S)-3,4-bis((4-(nonanoyloxy)-3-((nonanoyloxy)methyl)butanoyl)oxy)pyrrolidin-1-ium trfluoroacetate (35)

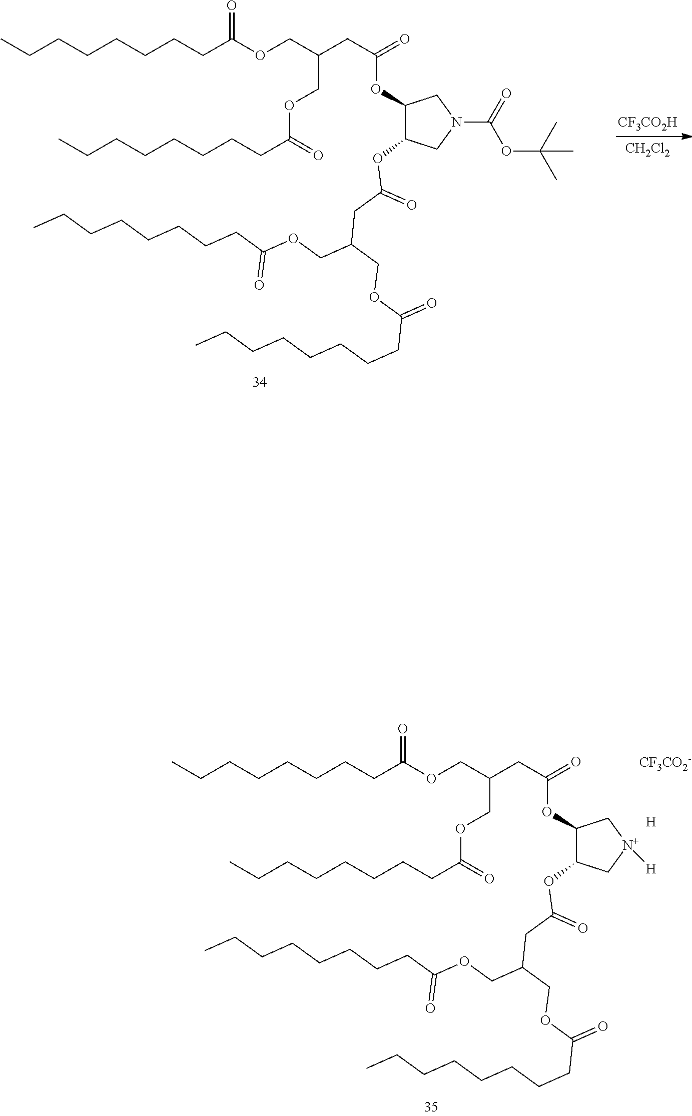

To a solution of 34 (8.00 g, 8.03 mmol), in CH₂Cl₂ (80 mL) under nitrogen at room temperature, was added CF₃CO₂H (20 mL, 29.78 g, 261 mmol) over a period of 15 minutes. The reaction mixture was allowed to stir for 3 hours after the addition was complete, then the solution was cast into 6.5% aq. K₂HPO₄ (160 mL). The organic phase was separated, washed with brine (2×80 mL), and dried over Na₂SO₄. Filtration and concentration in vacuo provided 35 (7.60 g, 7.52 mmol, HPLC purity 94%, 94% yield) as a light-yellow oil.

¹H-NMR (300 MHz, CDCl₃): δ=10.48 (brs, 2H), 5.35 (M, 2H), 4.18 (m, 8H), 3.75 (m, 2H), 3.62 (m, 2H), 2.42-2.62 (6H), 2.35 (m, 8H), 1.62 (m, 8H), 1.21-1.33 (40H), 0.90 (t, J=6.8 Hz, 12H); LCMS: RT 1.59, Calcd. for C₅₀H₉₀NO₁₂ 895.65. Found 896.6.

### Example 37: Synthesis ((((3S,4S)-1-(1H-imidazole-1-carbonyl)pyrrolidine-3,4-diyl)bis(oxy))bis(2-oxoethane-2,1-diyl))bis(propane-2,1,3-triyl) tetranonanoate (36)

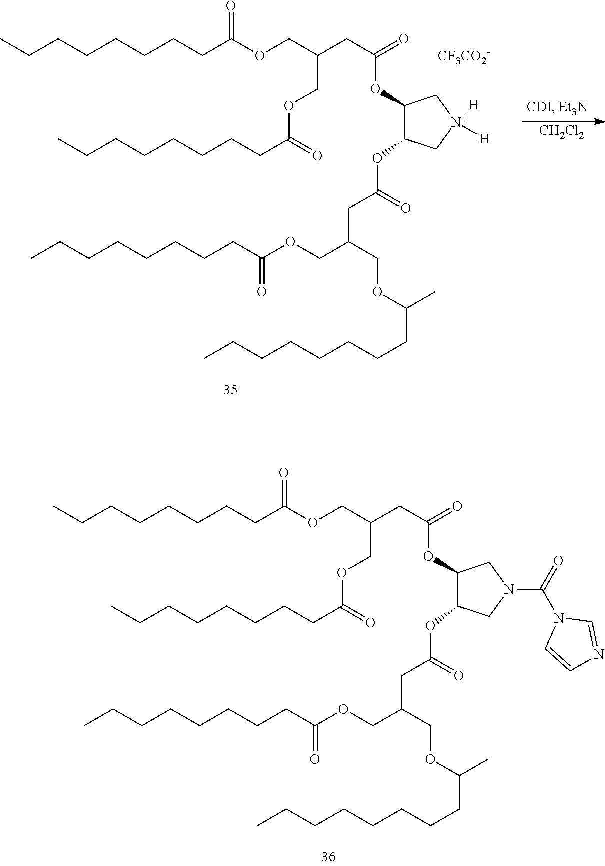

To a solution of 35 (7.50 g, 7.42 mmol) in dichloromethane (150 mL), at room temperature under nitrogen, was added in order CDI (12.22 g, 75.36 mmol) and Et₃N (6.10 g, 60.28 mmol). The reaction mixture was stirred for 16 hours at room temperature, then was cast into 0.5M aq. HCl (150 mL). The organic phase was separated, washed with brine (150 mL) and dried (Na₂SO₄). Filtration and concentration in vacuo afforded 36 which was dissolved in n-heptane (300 mL), the organic phase was washed with MeOH/water (4:1, 75 mL), water (75 mL), and dried (Na₂SO₄). Filtration and concentration in vacuo gave 36 (7.00 g, 7.07 mmol, 93% purity by HPLC, 95% yield) as a pale-yellow oil.

¹H-NMR (400 MHz, CDCl₃): δ=8.11 (brs, 1H), 7.42 (brs, 1H), 7.13 (brs, 1H), 5.29 (m, 2H), 4.06-4.25 (10H), 3.82 (m, 2H), 2.60 (m, 2H), 2.42 (m, 4H), 2.33 (m, 8H), 1.62 (m, 8H), 1.23-1.38 (40H), 0.90 (t, J=6.9 Hz, 12H); LCMS: RT 1.755, Calcd. for C₅₄H₉₁N₃O₁₃+H⁺ 990.66. Found 990.60.

### Example 38: Synthesis of ((((3S,4S)-1-((2-(dimethylamino)ethoxy)carbonyl)pyrrolidine-3,4-diyl)bis(oxy))bis(2-oxoethane-2,1-diyl))bis(propane-2,1,3-triyl) tetranonanoate (CICL-215)

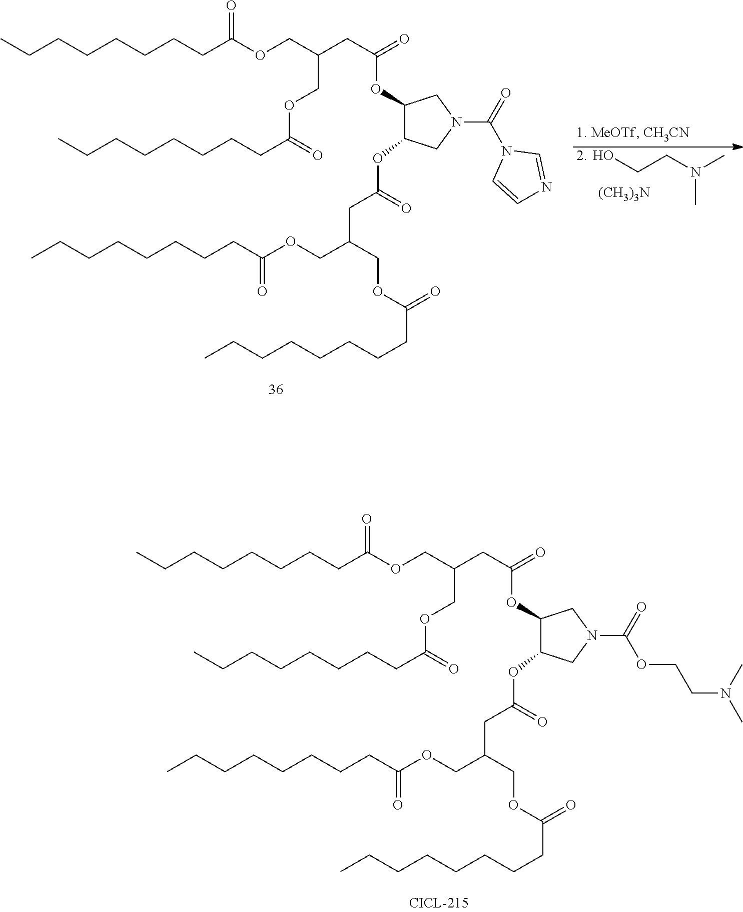

To a solution of 36 (5.00 g, 5.05 mmol), in CH3CN (100 mL), cooled in an ice-water bath under nitrogen was added MeOTf (0.91 g, 5.55 mmol) over a period of 5 minutes. The mixture was allowed to stir for 2 hours at 0° C., then a solution of trimethylamine in THF (2.0M, 7.57 mL, 22.20 mmol) was added over a period of 10 minutes followed by the addition of 2-dimethylaminoethanol (0.81 g, 9.09 mmol) in one portion. The mixture was stirred for 1 hour at 0° C., then, was warmed in a 70° C. oil bath, and was stirred for 48 hours. The reaction mixture was concentrated in vacuo, the residue was dissolved with n-heptane (550 mL) and the resulting solution was washed with MeOH/water (5:1, 2×100 mL), then the organic phase was dried over Na₂SO₄, and filtered. The filtrate was concentrated in vacuo and the residue was dissolved in CH₃CN (15 mL), and the crude product was purified by preparative reverse phase HPLC (XB Phenyl column, 50×250 mm, 10 μM; Mobile Phase A: water/0.1% TFA; Mobile Phase B: CH₃CN; flow rate 90 mL/min; Gradient 55% B to 90% B in 12 minutes; Wave Length: 205 nM). Qualified fractions were concentrated in vacuo, to remove CH₃CN, and the pH of the aq. residue was adjusted to 9.0 with 2% aq. Na₂CO₃. The aq. phase was extracted with n-heptane (2×275 mL), and the combined organic phases were washed with MeOH/water (4:1, 3×110 mL), water (145 mL) and dried (Na₂SO₄). Filtration and concentration in vacuo gave CICL-215 (2.02 g, 2.00 mmol, 97% purity by HPLC, 40% yield) as a clear, colorless, viscous oil.

¹H-NMR (300 MHz, CDCl3): δ=5.22 (m, 2H), 4.22 (m, 2H), 4.11 (m, 8H), 3.76 (dd, J=12.9, 4.2 Hz, 2H), 3.59 (m, 2H), 2.49-2.64 (4H), 2.43 (m, 4H), 2.28-2.38 (14H), 1.62 (m, 8H), 1.21-1.39 (40H), 0.90 (m, 12H); LCMS: RT 1.575, Calcd. for C₅₅H₉₈N₂O₁₄+H⁺ 1011.71. Found 1011.60.

### Example 39: ((((3R,5R)-1-(tert-butoxycarbonyl)piperidine-3,5-diyl)bis(oxy))bis(2-oxoethane-2,1-diyl))bis(propane-2,1,3-triyl) tetranonanoate (38)

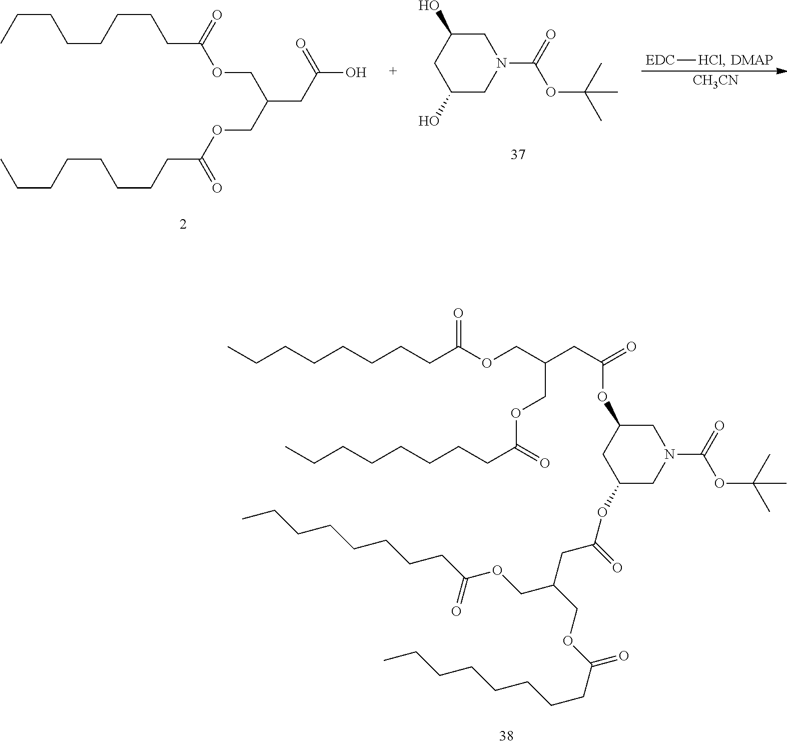

To a solution of 37 (Tetrahedron 2011, 67, 1485, 2.20 g, 10.14 mmol) in acetonitrile (44 mL), at room temperature under nitrogen, was added in order. 2 (8.60 g, 20.78 mmol), DMAP (1.24 g, 10.14 mmol), and EDC-HCl (4.87 g, 23.34 mmol). The reaction mixture was stirred for 18 hours at room temperature then was concentrated in vacuo. The residue was dissolved in n-heptane (440 mL) and the resulting solution was extracted with MeOH/water (10:1, 2×110 mL). The solvent was removed in vacuo and the residue was dissolved in dichloromethane 44 mL) and silica gel (20g, Type: ZCX-2, 100-200 mesh) was added. The solvent was then removed in vacuo (temperature <35° C.). The resulting 38 impregnated silica gel was placed atop a combi-flash column charged with silica gel (120g, Type: ZCX-2, 100-200 mesh), and the combi-flash column was eluted with a gradient of n-heptane/ethyl acetate (100:0 to 90:10). Qualified fractions were combined and concentrated in vacuo to furnish 38 which was further purified by reverse phase prep-HPLC (Column: XB Phenyl 50×250 mm, 10m; Mobile Phase A: water (0.1% CF3CO2H), Mobile Phase B: CH₃CN; Flow Rate: 90 mL/min; Gradient: 50% B to 95% B over 10 minutes; Wave Length: 205 nM). Qualified fractions were pooled, the acetonitrile was removed in vacuo and the resulting solution was extracted with n-heptane (440 mL). The organic phase was washed with MeOH/water (4:1, 2×110 mL), water (110 mL), and dried (Na₂SO₄). Filtration and concentration in vacuo gave 38 (7.10 g, 7.02 mmol, HPLC purity 98%, 69% yield) as a viscous, colorless oil.

¹H-NMR (300 MHz, CDCl3): δ=5.05 (brs, 2H), 4.12 (m, 8H), 3.30-3.75 (4H), 2.58 (m, 2H), 2.40 (m, 4H), 2.34 (m, 8H), 2.00 (brm, 2H), 1.62 (m, 8H), 1.49 (s, 9H), 1.20-1.37 (40H), 0.88 (t, J=6.8 Hz, 12H); LCMS: RT 2.017, Calcd. for C₅₆H₉₉NO₁₄+Na+ 1032.70. Found 1032.60.

### Example 40: Synthesis of (3R,5R)-3,5-bis((4-(nonanoyloxy)-3-((nonanoyloxy)methyl)butanoyl)oxy)piperidin-1-ium trifluoroacetate (39)

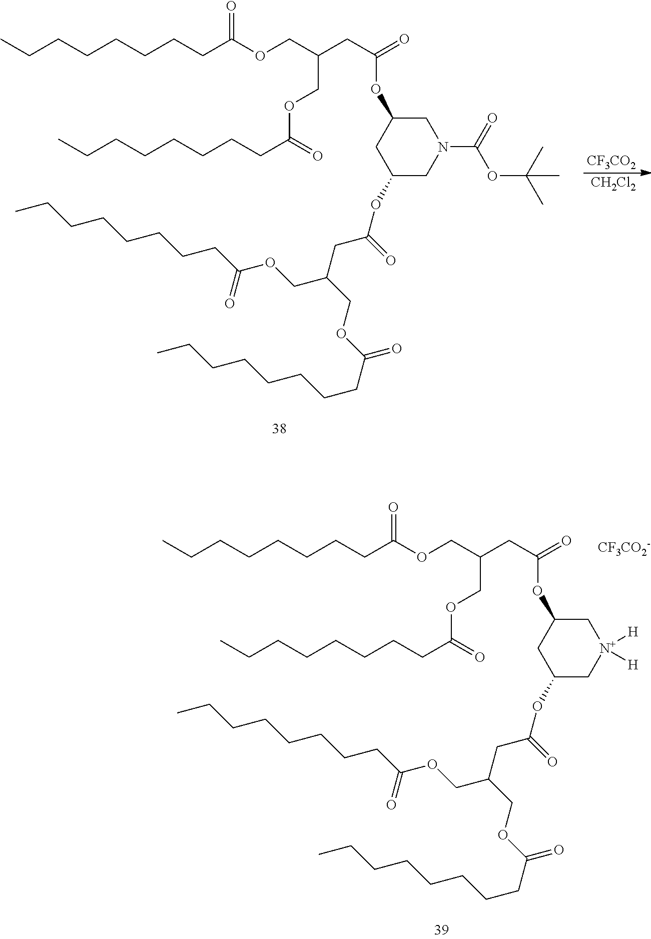

To a solution of 38 (6.00 g, 5.94 mmol), in CH₂Cl₂ (30 mL) under nitrogen, cooled to 20° C., was added CF₃CO₂H (12 mL, 17.87 g, 157 mmol) over a period of 15 minutes. The reaction mixture was allowed to stir for 3 hours at 20° C. after the addition was complete, then the solution was concentrated in vacuo and the residue was dissolved in n-heptane (600 mL). The resulting solution was washed with brine (2×120 mL), then the organic phase was concentrated in vacuo to provide 39 (5.40 g, 5.27 mmol, HPLC purity 97%, 89% yield) as a pale-yellow oil.

¹H-NMR (400 MHz, CDCl3): δ=5.30 (m, 2H), 4.11 (m, 8H), 3.38 (dd, J=13.6, 3.6 Hz, 2H), 3.23 (m, 2H), 2.40-2.58 (6H), 2.38 (m, 8H), 2.18 (m, 2H), 1.63 (m, 8H), 1.23-1.38 (40H), 0.90 (t, J=7.2 Hz, 12H); LCMS: RT 1.611, Calcd. for C₅₁H₉₂NO₁₂ 910.66. Found 910.60.

### Example 41: Synthesis of ((((3R,5R)-1-(1H-Imidazole-1-carbonyl)piperidine-3,5-diyl)bis(oxy))bis(2-oxoethane-2,1-diyl))bis(propane-2,1,3-triyl) tetranonanoate (40)

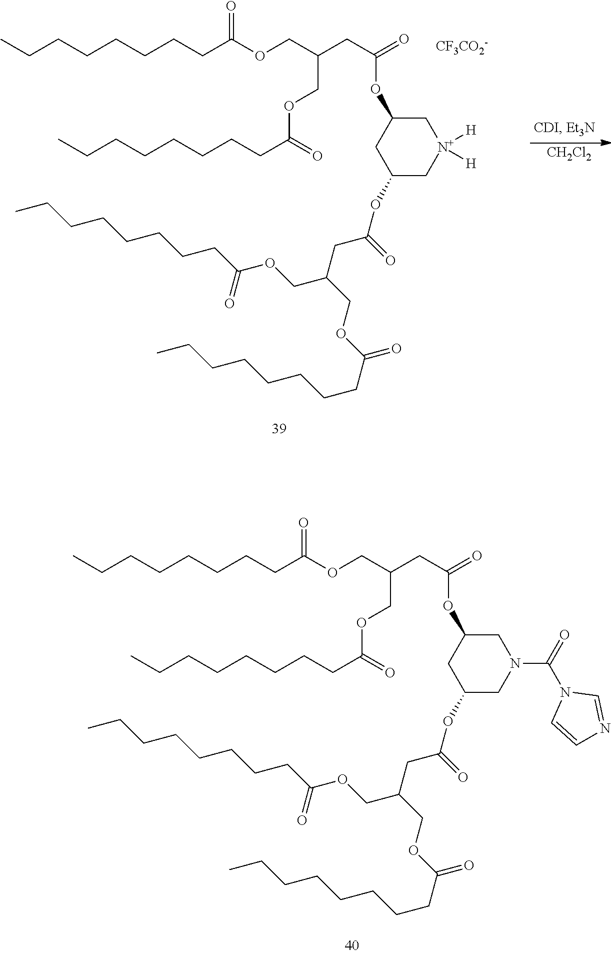

To a solution of 39 (5.40 g, 5.33 mmol) in dichloromethane (108 mL), at room temperature under nitrogen, was added in order: CDI (8.64 g, 53.60 mmol) and Et₃N (4.33 g, 42.86 mmol). The reaction mixture was stirred for 18 hours at room temperature, then was cast into 0.5M aq. HCl (125 mL). The aq. layer was extracted with dichloromethane (2×270 mL) and the combined organic phases were concentrated in vacuo to afford crude 40 which was dissolved in n-heptane (675 mL). The organic phase was washed with MeOH/water (5:1, 2×270 mL), and was concentrated in vacuo, providing 40 (4.90 g, 4.68 mmol, 94% purity by HPLC, 88% yield) as a pale-yellow oil.

¹H-NMR (400 MHz, CDCl3): δ=7.98 (brs, 1H), 7.30 (s, 1H), 7.14 (s, 1H), 5.18 (m, 2H), 4.10 (m, 8H), 3.80 (m, 2H), 3.62 (m, 2H), 2.54 (m, 2H), 2.46 (m, 4H), 2.32 (m, 8H), 2.14 (m, 2H), 1.62 (m, 8H), 1.22-1.38 (40H), 0.89 (t, J=6.8 Hz, 12H); LCMS: RT 1.800, Calcd. for C₅₅H₉₃N₃O₁₃+H⁺ 1004.68. Found 1004.60.

### Example 42: Synthesis of ((((3R,5R)-1-((2-(dimethylamino)ethoxy)carbonyl)piperidine-3,5-diyl)bis(oxy))bis(2-oxoethane-2,1-diyl))bis(propane-2,1,3-triyl) tetranonanoate (CICL-220)

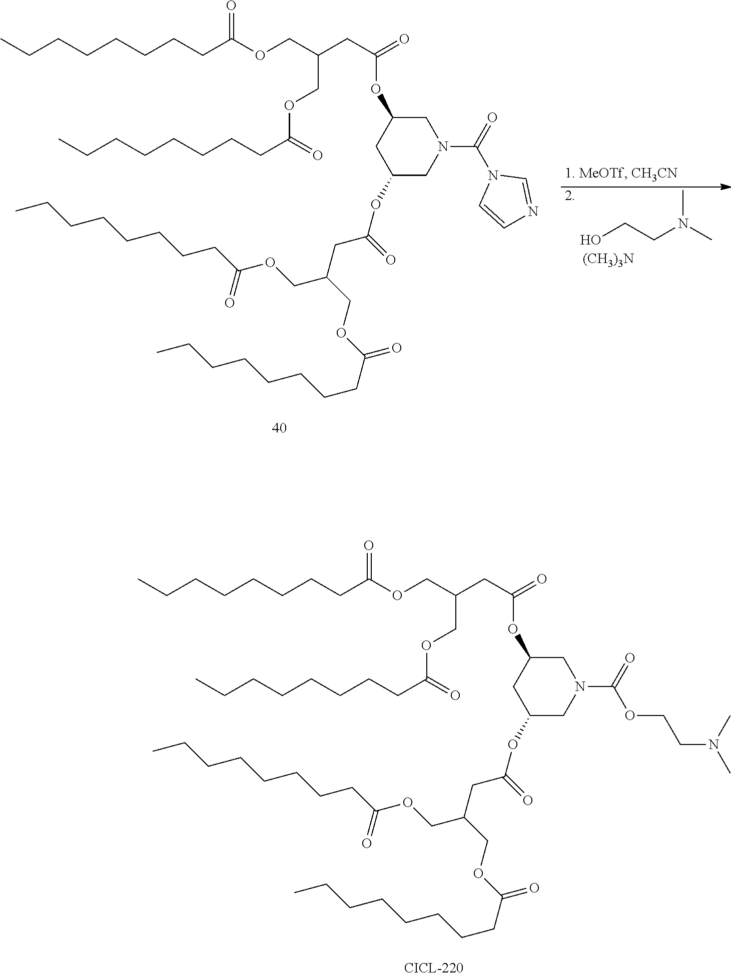

A solution of 40 (5.50 g, 6.57 mmol) in acetonitrile (75 mL), under nitrogen, was cooled in an ice-water bath. To this cooled solution was added MeOTf (1.08 g, 6.57 mmol) over a period of 5 minutes. The mixture was stirred for 2 hours, then Me₃N (2M in THF, 10.9 mL, 21.8 mmol) was added over a period of 10 minutes, this was followed by the addition of 2-dimethylaminoethanol (0.73 g, 8.22 mmol) in one portion. The reaction mixture was stirred for 1 hour at 0° C., then it was placed in a 60° C. oil bath and stirred for 120 hours. After cooling to room temperature, the mixture was concentrated in vacuo and the residue was dissolved in n-heptane (550 mL). The solution was washed with MeOH/water (5:1, 2×100 mL), and the organic phase was dried over Na₂SO₄. Filtration and concentration in vacuo provided crude CICL-220 (4.80g) which was purified by preparative reverse phase HPLC (Waters C18 column; mobile phase A (0.1% TFA in water), mobile phase B (acetonitrile); with a gradient of 50% to 90% B over 12 minutes; UV 205 nM). Qualified fractions were combined, and the acetonitrile was removed in vacuo. The resulting mixture was adjusted to pH 8 with 2% aq. Na₂CO₃ and extracted with n-heptane (2×275 mL). The combined organic phases were washed with MeOH/water (4:1, 3×110 mL), water (110 mL), and dried (Na₂SO₄). Filtration and concentration in vacuo afforded CICL-220 (2.35 g, 2.29 mmol, 97% purity by HPLC, 35% yield) as a clear, colorless oil.

¹H-NMR (400 MHz, CDCl3): δ=5.06 (brm, 2H), 4.22 (m, 2H), 4.10 (m, 8H), 3.73 (brm, 1H), 3.58 (brm, 2H), 3.41 (brm, 1H), 2.58 (m, 4H), 2.41 (m, 4H), 2.24-2.34 (14H), 1.99 (brm, 2H), 1.62 (m, 8H), 1.20-1.37 (40H), 0.87 (t, J=6.6 Hz, 12H); LCMS: RT 1.588, Calcd. for C₅₆H₁₀₀N₂O₁₄+H⁺ 1025.73. Found 1025.70.

### Example 43: Synthesis of (((Rel-(3R,6S)-1-(tert-butoxycarbonyl)-2,3,6,7-tetrahydro-1H-azepine-3,6-diyl)bis(oxy))bis(2-oxoethane-2,1-diyl))bis(propane-2,1,3-triyl) tetranonanoate (42)

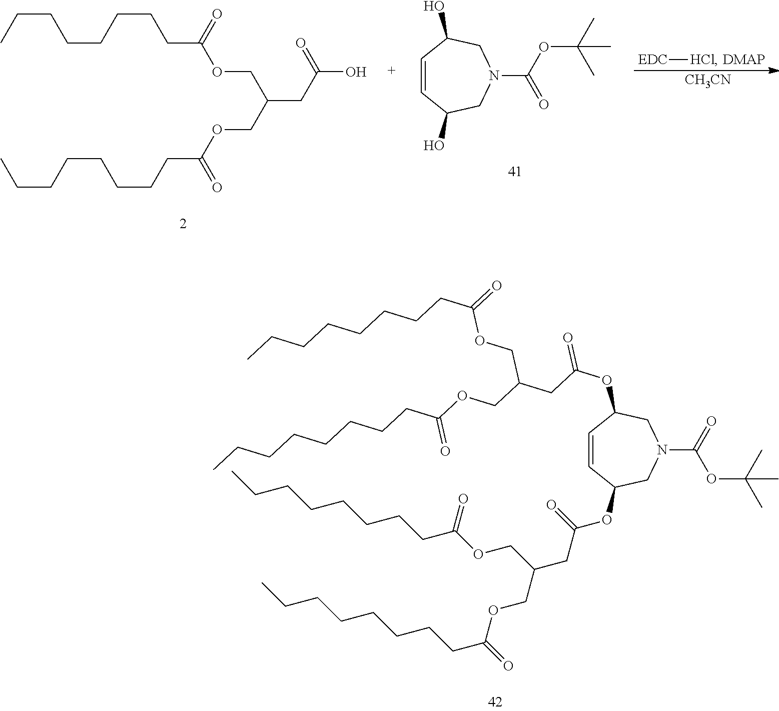

To a solution of 41 (Nature Communications 2019, 10, 21, 1.42 g, 6.20 mmol) in acetonitrile (50 mL), at room temperature under nitrogen, was added in order 2 (5.26 g, 12.68 mmol), DMAP (0.76 g, 6.20 mmol), and EDC-HCl (2.98 g, 15.50 mmol). The reaction mixture was stirred for 18 hours at room temperature then was concentrated in vacuo. The residue was dissolved in n-heptane (200 mL) and the resulting solution was extracted with MeOH/water (10:1, 2×100 mL). The solvent was removed in vacuo and the residue was dissolved in dichloromethane (15 mL) and silica gel (20g, Type: ZCX-2, 100-200 mesh) was added. The solvent was then removed in vacuo (temperature <35° C.). The resulting 42 impregnated silica gel was placed atop a combi-flash column charged with silica gel (120g, Type: ZCX-2, 100-200 mesh), and the combi-flash column was eluted with a gradient of n-heptane/ethyl acetate (40:1 to 25:1). Qualified fractions were combined and concentrated in vacuo to furnish 42 (5.20 g, 5.09 mmol, HPLC purity 92%, 84% yield) as a pale-yellow oil.

¹H-NMR (400 MHz, CDCl3): δ=5.69 (s, 2H), 5.48 (m, 2H), 4.12 (m, 8H), 4.08 (m, 1H), 3.92 (m, 1H), 3.17 (m, 2H), 2.59 (m, 2H), 2.48 (m, 4H), 2.31 (m, 8H), 1.63 (m, 8H), 1.50 (s, 9H), 1.22-1.35 (40H), 0.89 (m, 12H); LCMS: RT 2.230, Calcd. for C₅₇H₉₉NO₁₄+Na+ 1044.70. Found 1044.80.

### Example 44: Synthesis of Rel-(3R,6S)-3,6-bis((4-(nonanoyloxy)-3-((nonanoyloxy)methyl)butanoyl)oxy)-2,3,6,7-tetrahydro-1H-azepin-1-ium trifluoroacetate (43)

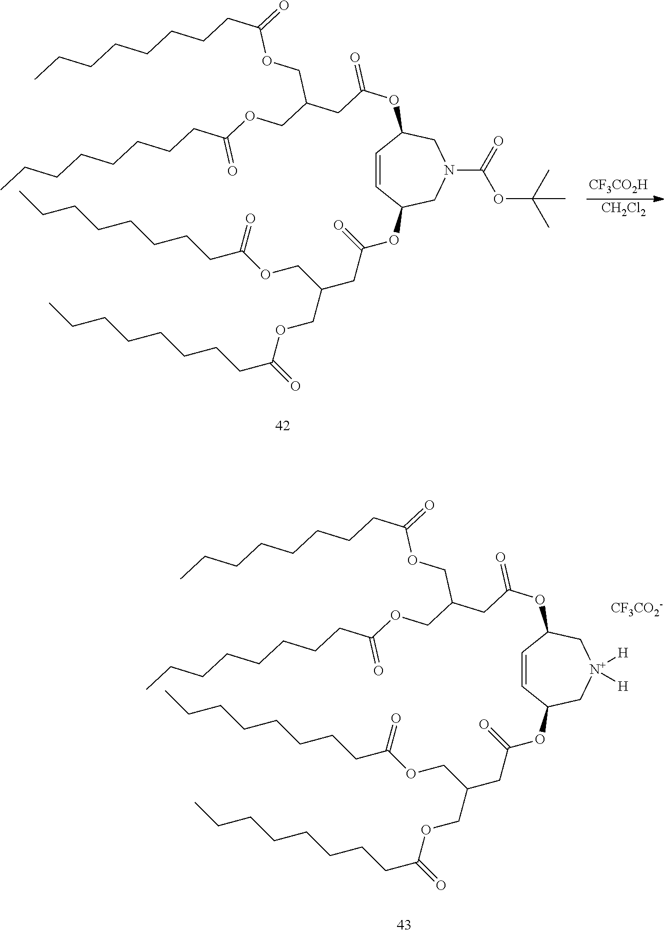

To a solution of 42 (5.10 g, 5.10 mmol), in CH₂Cl₂ (75 mL) under nitrogen, at room temperature, was added CF₃CO₂H (25 mL, 37.23 g, 326 mmol) over a period of 15 minutes. The reaction mixture was allowed to stir for 18 hours at room temperature after the addition was complete, then the solution was concentrated in vacuo and the residue was dissolved in n-heptane (500 mL). The resulting solution was washed with brine (2×200 mL), then the organic phase was concentrated in vacuo to provide 43 (5.03 g, 4.85 mmol, HPLC purity 93%, 95% yield) as a pale-yellow oil.

¹H-NMR (400 MHz, CDCl3): δ=6.00 (s, 2H), 5.74 (m, 2H), 4.13 (m, 8H), 3.52 (m, 4H), 2.57 (m, 2H), 2.46 (m, 4H), 2.32 (m, 8H), 1.62 (m, 8H), 1.20-1.37 (40H), 0.89 (m, 12H); LCMS: RT 1.77, Calcd. for C21H₉₂NO₁₂ 922.66. Found 922.8.

### Example 45: Synthesis of (((Rel-(3R,6S)-1-(1H-Imidazole-1-carbonyl)-2,3,6,7-tetrahydro-1H-azepine-3,6-diyl)bis(oxy))bis(2-oxoethane-2,1-diyl))bis(propane-2,1,3-triyl) tetranonanoate (44)

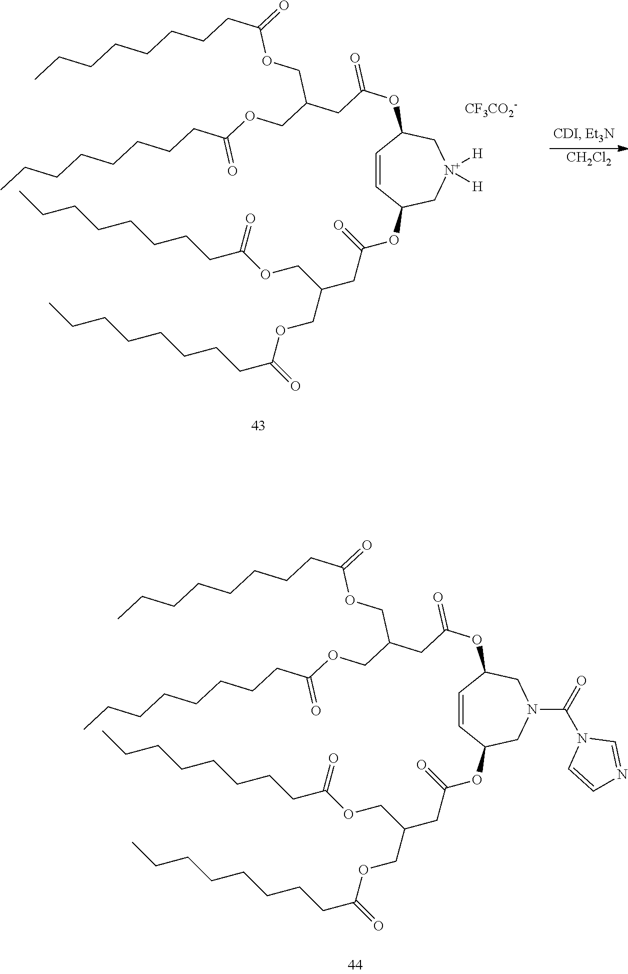

To a solution of 43 (5.00 g, 4.82 mmol) in dichloromethane (100 mL), at room temperature under nitrogen, was added in order: CDI (7.94 g, 49.00 mmol) and Et₃N (3.97 g, 39.20 mmol). The reaction mixture was stirred for 18 hours at room temperature, then was cast into 0.5M aq. HCl (125 mL). The aq. layer was extracted with dichloromethane (2×200 mL) and the combined organic phases were concentrated in vacuo to afford crude 44 which was dissolved in n-heptane (300 mL). The organic phase was washed with MeOH/water (8:1, 2×200 mL), and was concentrated in vacuo, providing 44 (4.30 g, 4.23 mmol, 96% purity by HPLC, 88% yield) as a yellow oil.

¹H-NMR (300 MHz, CDCl3): δ=8.01 (s, 1H), 7.28 (s, 1H), 7.15 (s, 1H), 5.85 (m, 2H), 5.61 (m, 2H), 4.00-4.22 (10H), 3.61-3.73 (2H), 2.48 (m, 2H), 2.45 (m, 4H), 2.27-2.38 (8H), 1.61 (m, 8H), 1.20-1.39 (40H), 0.89 (m, 12H); LCMS: RT 1.777, Calcd. for C₅₆H₉₃N₃O₁₃+H⁺ a 1016.68. Found 1016.70.

### Example 46: Synthesis of (((Rel-(3R,6S)-1-((2-(dimethylamino)ethoxy)carbonyl)-2,3,6,7-tetrahydro-1H-azepine-3,6-diyl)bis(oxy))bis(2-oxoethane-2,1-diyl))bis(propane-2,1,3-triyl) tetranonanoate (CICL-219)

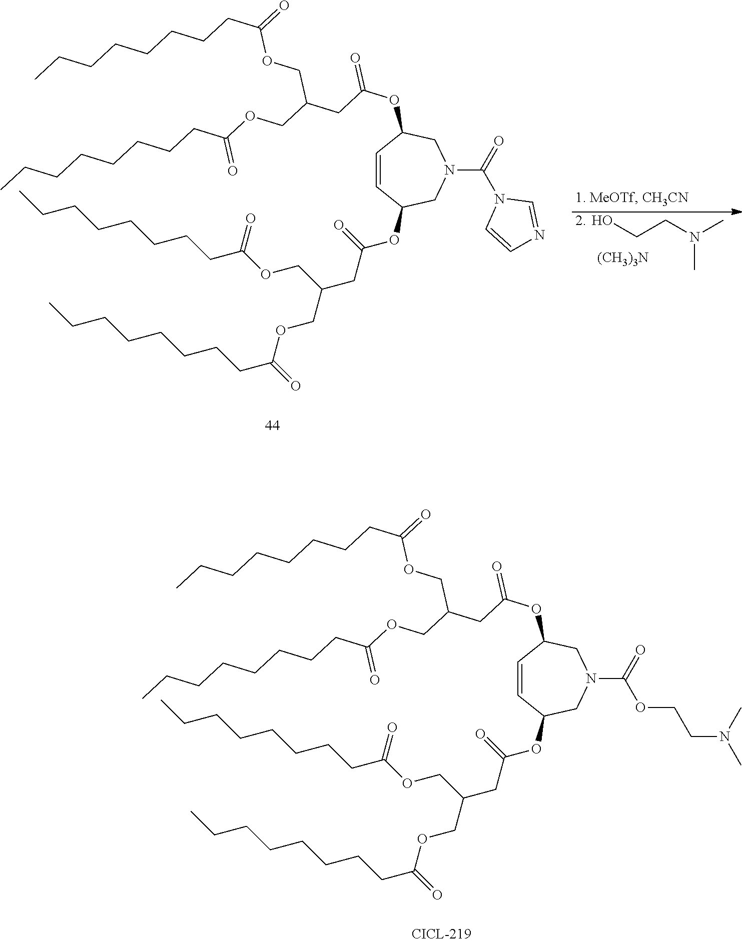

A solution of 44 (4.20 g, 4.13 mmol) in acetonitrile (60 mL), under nitrogen, was cooled in an ice-water bath. To this cooled solution was added MeOTf (0.81 g, 5.00 mmol) over a period of 5 minutes. The mixture was stirred for 2 hours, then Me₃N (2M in THF, 6.20 mL, 12.40 mmol) was added over a period of 10 minutes, and this was followed by the addition of 2-dimethylaminoethanol (0.55 g, 6.20 mmol) in one portion. The reaction mixture was stirred for 1 hour at 0° C., then it was placed in a 60° C. oil bath and stirred for 48 hours. After cooling to room temperature, the mixture was concentrated in vacuo and the residue was dissolved in n-heptane (300 mL). The solution was washed with MeOH/water (5:1, 2×50 mL), and the organic phase was dried over Na₂SO₄. Filtration and concentration in vacuo provided crude CICL-219 which was dissolved in dichloromethane (20 mL), and silica gel (20g, Type: ZCX-2, 100-200 mesh) was added. The solvent was then removed in vacuo (temperature <35° C.). The resulting CICL-219 impregnated silica gel was placed atop a combi-flash column charged with silica gel (120g, Type: ZCX-2, 100-200 mesh), and the combi-flash column was eluted with a gradient of n-heptane/ethyl acetate (100:0 to 40:70). Qualified fractions were combined and concentrated in vacuo to furnish CICL-219 which was dissolved in n-heptane (300 mL), washed with MeOH/water (5:1, 2×50 mL), water (2×50 mL), and dried (Na₂SO₄). Filtration and concentration in vacuo provided CICL-219 (1.49 g, 1.44 mmol, 98% purity by HPLC, 35% yield) as a pale-yellow oil.

¹H-NMR (400 MHz, CDCl3): δ=5.69 (s, 2H), 5.52 (brm, 2H), 4.27 (m, 2H), 3.98-4.20 (10H), 3.23 (m, 2H), 2.62 (m, 4H), 2.45 (m, 4H), 2.28-2.38 (14H), 1.64 (m, 8H), 1.21-1.39 (40H), 0.90 (m, 12H); LCMS: RT 1.652, Calcd. for C₅₇H₁₀₀N₂O₄+H⁺ 1037.72. Found 1037.60.

### Example 47: Synthesis of (((Rel-(3S,6S)-1-(tert-butoxycarbonyl)-2,3,6,7-tetrahydro-1H-azepine-3,6-diyl)bis(oxy))bis(2-oxoethane-2,1-diyl))bis(propane-2,1,3-triyl) tetranonanoate (46)

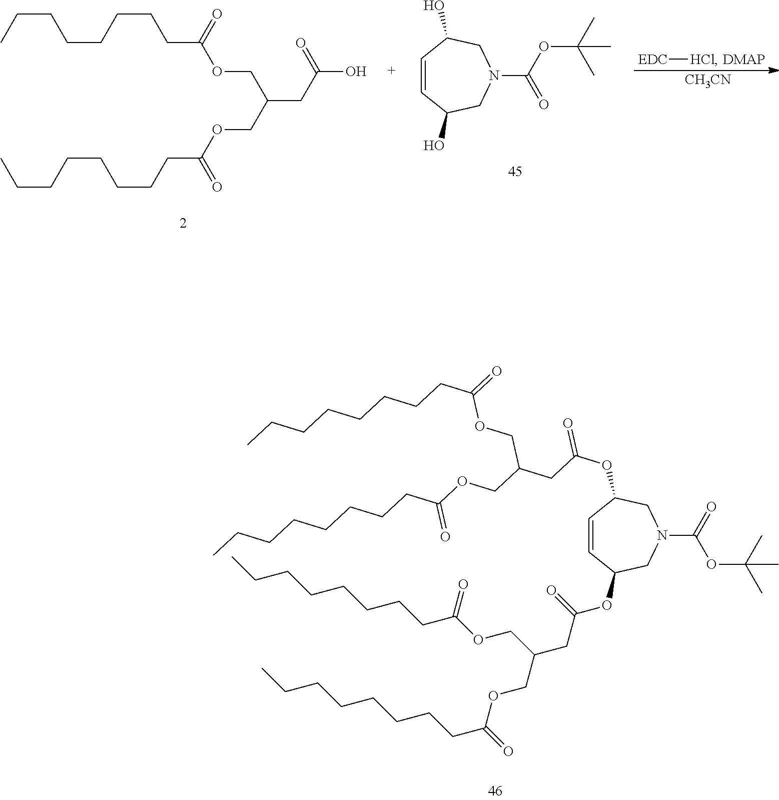

To a solution of 45 (Nature Communications 2019, 10, 21, 1.45 g, 6.32 mmol) in acetonitrile (50 mL), at room temperature under nitrogen, was added in order 2 (5.37 g, 13.00 mmol), DMAP (0.78 g, 6.30 mmol), and EDC-HCl (3.04 g, 15.80 mmol). The reaction mixture was stirred for 18 hours at room temperature then was concentrated in vacuo. The residue was dissolved in n-heptane (100 mL) and the resulting solution was extracted with MeOH/water (10:1, 2×100 mL). The solvent was removed in vacuo and the residue was dissolved in dichloromethane (15 mL) and silica gel (20g, Type: ZCX-2, 100-200 mesh) was added. The solvent was then removed in vacuo (temperature <35° C.). The resulting 46 impregnated silica gel was placed atop a combi-flash column charged with silica gel (120g, Type: ZCX-2, 100-200 mesh), and the combi-flash column was eluted with a gradient of n-heptane/ethyl acetate (40:1 to 25:1). Qualified fractions were combined and concentrated in vacuo to furnish 46 (5.32 g, 5.20 mmol, HPLC purity 95%, 82% yield) as a pale-yellow oil.

¹H-NMR (400 MHz, CDCl3): δ=5.78-5.88 (2H), 5.52 (brm, 1H), 5.42 (brm, 1H), 4.13 (m, 8H), 3.90 (m, 1H), 3.82 (m, 1H), 3.49 (m, 1H), 3.40 (m, 1H), 2.59 (m, 2H), 2.44 (m, 4H), 2.33 (m, 8H), 1.62 (m, 8H), 1.49 (s, 9H), 1.21-1.38 (40H), 0.89 (t, J=6.8 Hz, 12H); LCMS: RT 2.037, Calcd. for C₅₇H₉₉NO₁₄+Na⁺ 1044.70. Found 1044.70.

### Example 48: Synthesis of Rel-(3S,6S)-3,6-bis((4-(nonanoyloxy)-3-((nonanoyloxy)methyl)butanoyl)oxy)-2,3,6,7-tetrahydro-1H-azepin-1-ium trifluoroacetate (47)

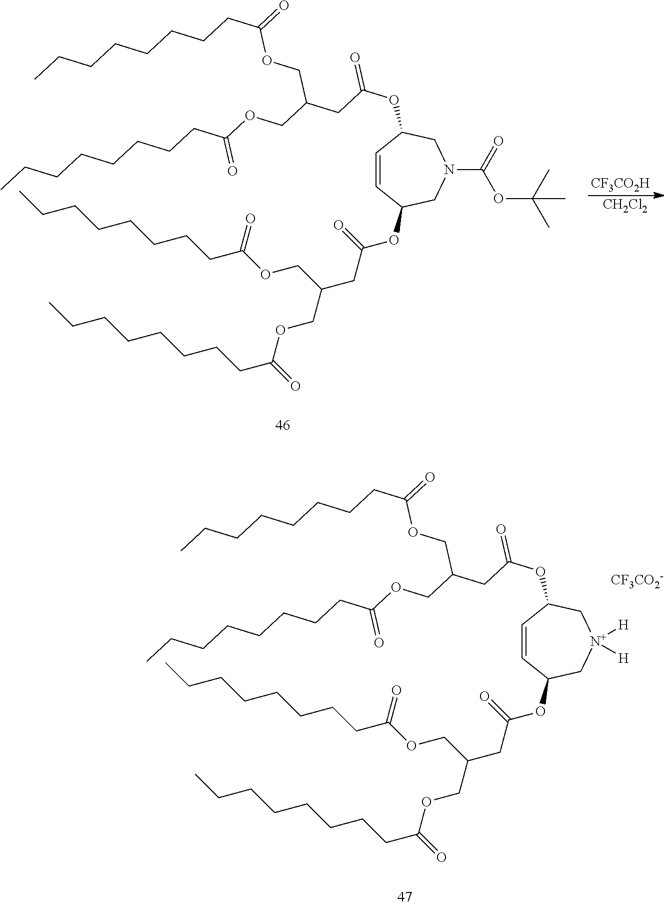

To a solution of 46 (5.20 g, 5.10 mmol), in CH₂Cl₂ (75 mL) under nitrogen, at room temperature, was added CF₃CO₂H (25 mL, 37.23 g, 326 mmol) over a period of 15 minutes. The reaction mixture was allowed to stir for 18 hours at room temperature after the addition was complete, then the solution was concentrated in vacuo and the residue was dissolved in n-heptane (500 mL). The resulting solution was washed with brine (2×200 mL), then the organic phase was concentrated in vacuo to provide 47 (5.16 g, 4.98 mmol, HPLC purity 90%, 98% yield) as a pale-yellow oil.

¹H-NMR (300 MHz, CDCl3): δ=6.10-6.18 (2H), 5.62 (m, 2H), 5.31 (s, 2H), 4.10 (m, 8H), 3.54 (m, 4H), 2.47-2.62 (6H), 2.30 (m, 8H), 1.63 (m, 8H), 1.20-1.37 (40H), 0.90 (m, 12H); LCMS: RT 1.767, Calcd. for C₂H₉₂NO₁₂ 922.66. Found 922.8.

### Example 49: Synthesis of (((Rel-(3S,6S)-1-(1H-Imidazole-1-carbonyl)-2,3,6,7-tetrahydro-1H-azepine-3,6-diyl)bis(oxy))bis(2-oxoethane-2,1-diyl))bis(propane-2,1,3-triyl) tetranonanoate (48)

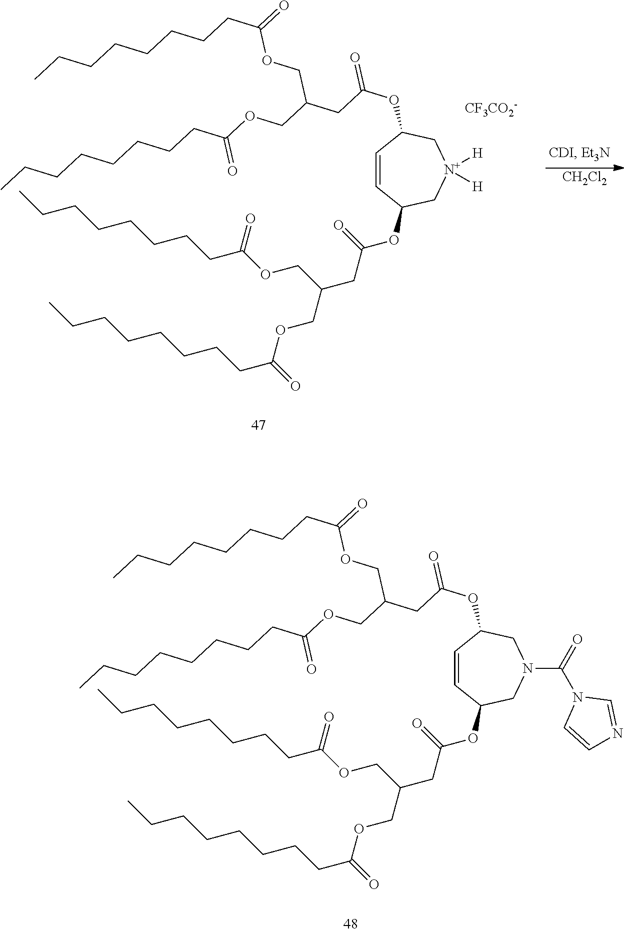

To a solution of 47 (5.10 g, 4.92 mmol) in dichloromethane (100 mL), at room temperature under nitrogen, was added in order: CDI (8.10 g, 50.00 mmol) and Et₃N (4.05 g, 40.00 mmol). The reaction mixture was stirred for 18 hours at room temperature, then was cast into 0.5M aq. HCl (125 mL). The aq. layer was extracted with dichloromethane (2×300 mL) and the combined organic phases were concentrated in vacuo to afford crude 48 which was dissolved in n-heptane (300 mL). The organic phase was washed with MeOH/water (8:1, 2×200 mL), water (200 mL), and was concentrated in vacuo, providing 48 (4.30 g, 4.23 mmol, 95% purity by HPLC, 86% yield) as a yellow oil.

¹H-NMR (300 MHz, CDCl3): δ=7.99 (s, 1H), 7.31 (s, 1H), 7.14 (s, 1H), 5.80-5.90 (2H), 5.68 (m, 2H), 4.12 (m, 8H), 3.95 (m, 2H), 3.82 (m, 2H), 2.57 (m, 2H), 2.41 (m, 4H), 2.32 (m, 8H), 1.62 (m, 8H), 1.20-1.40 (40H), 0.89 (m, 12H); LCMS: RT 1.945, Calcd. for C₅₆H₉₃N₃O₁₃+H⁺ a 1016.68. Found 1016.70.

### Example 50: Synthesis of (((Rel-(3S,6S)-1-((2-(dimethylamino)ethoxy)carbonyl)-2,3,6,7-tetrahydro-1H-azepine-3,6-diyl)bis(oxy))bis(2-oxoethane-2,1-diyl))bis(propane-2,1,3-triyl) tetranonanoate (CICL-218)

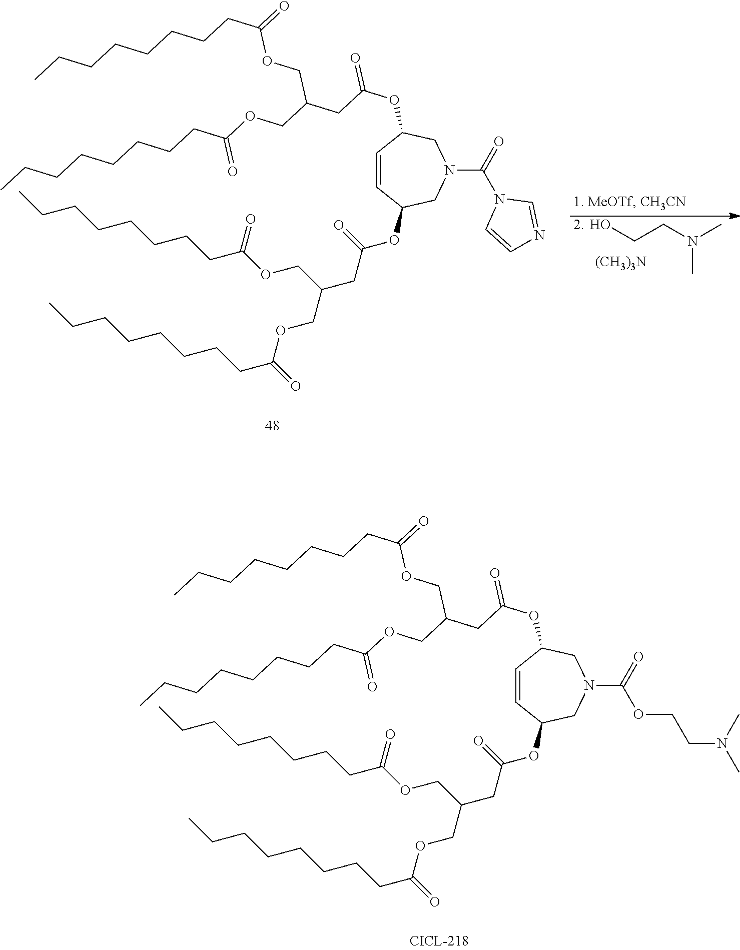

A solution of 48 (4.20 g, 4.13 mmol) in acetonitrile (60 mL), under nitrogen, was cooled in an ice-water bath. To this cooled solution was added MeOTf (0.81 g, 5.00 mmol) over a period of 5 minutes. The mixture was stirred for 2 hours, then Me₃N (2M in THF, 6.20 mL, 12.40 mmol) was added over a period of 10 minutes, and this was followed by the addition of 2-dimethylaminoethanol (0.55 g, 6.20 mmol) in one portion. The reaction mixture was stirred for 1 hour at 0° C., then it was placed in a 60° C. oil bath and stirred for 48 hours. After cooling to room temperature, the mixture was concentrated in vacuo and the residue was dissolved in n-heptane (300 mL). The solution was washed with MeOH/water (5:1, 2×50 mL), and the organic phase was dried over Na₂SO₄. Filtration and concentration in vacuo provided crude CICL-218 which was dissolved in n-heptane (300 mL). The organic phase was washed with MeOH/water (5:1, 2×50 mL), water (50 mL), and dried (Na₂SO₄). Filtration and concentration in vacuo gave crude CICL-218 which was dissolved with dichloromethane (20 mL), and silica gel (20g, Type: ZCX-2, 100-200 mesh) was added. The solvent was then removed in vacuo (temperature <35° C.). The resulting CICL-218 impregnated silica gel was placed atop a combi-flash column charged with silica gel (120g, Type: ZCX-2, 100-200 mesh), and the combi-flash column was eluted with a gradient of n-heptane/ethyl acetate (100:0 to 40:70). Qualified fractions were combined and concentrated in vacuo to furnish CICL-218 which was dissolved in n-heptane (300 mL), washed with MeOH/water (5:1, 2×50 mL), water (2×50 mL), and dried (Na₂SO₄). Filtration and concentration in vacuo provided CICL-218 (1.16 g, 1.12 mmol, 97% purity by HPLC, 27% yield) as a pale-yellow oil.

¹H-NMR (400 MHz, CDCl3): δ=5.77-5.89 (2H), 5.50 (m, 2H), 4.27 (m, 1H), 4.07-4.20 (9H), 3.83 (m, 2H), 3.64 (m, 1H), 3.53 (m, 1H), 2.60 (m, 4H), 2.42 (m, 4H), 2.24-2.33 (14H), 1.62 (m, 8H), 1.21-1.37 (40H), 0.89 (m, 12H); LCMS: RT 1.652, Calcd. for C₅₇H₁₀₀N₂O₁₄+H⁺ 1037.72. Found 1037.70.

### Example 51: Synthesis of tert-Butyl Rei-(3R,5S)-3,5-dihydroxypiperidine-1-carboxylate (50)

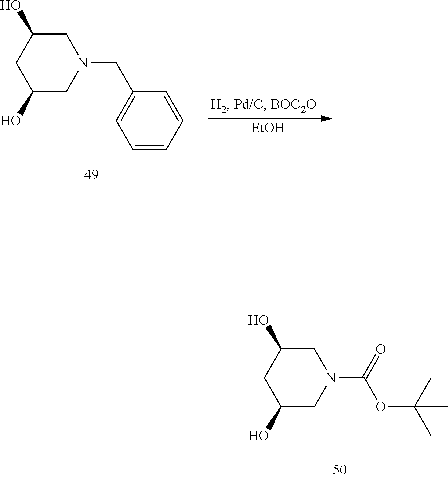

To a solution of 49 (WO2013149362, 8.00 g, 38.6 mmol) in EtOH (160 mL) was added BOC₂0 (12.64 g, 57.9 mmol) and Pd/C (1.60 g, 20 wt %). The resulting solution was placed under hydrogen (15 atm) at room temperature for 24 hours. The mixture was filtered through a pad of Celite®, the filter cake rinsed with EtOH (3×50 mL) and the combined filtrates were concentrated in vacuo to provide crude 50 as a pale yellow oil. Crude 50 was dissolved in THF (30 mL) and purified by reverse phase preparative HPLC (Column: XB Phenyl, 50×250 mm, 10 μm; Mobile Phase A: water (0.1% TFA), Mobile Phase B: acetonitrile; Flow rate: 90 mL/min; Gradient 0% B to 50% B in 10 min, 50% collected; Wave Length: 200 nM). Qualified fractions were pooled and concentrated in vacuo to yield 50 (4.20 g, 19.33 mmol, 50% yield), as a clear, colorless oil.

¹H-NMR (300 MHz, CDCl3): δ=4.31 (brt, J=5.00 Hz, 1H), 4.17 (brs, 2H), 3.91-4.20 (2H), 3.41-3.63 (3H), 2.35 (m, 1H), 1.89 (brd, J=14.1 Hz, 1H).

### Example 52: Synthesis of (((Rel-(3R,5S)-1-(tert-Butoxycarbonyl)piperidine-3,5-diyl)bis(oxy))bis(2-oxoethane-2,1-diyl))bis(propane-2,1,3-triyl) tetranonanoate (51)

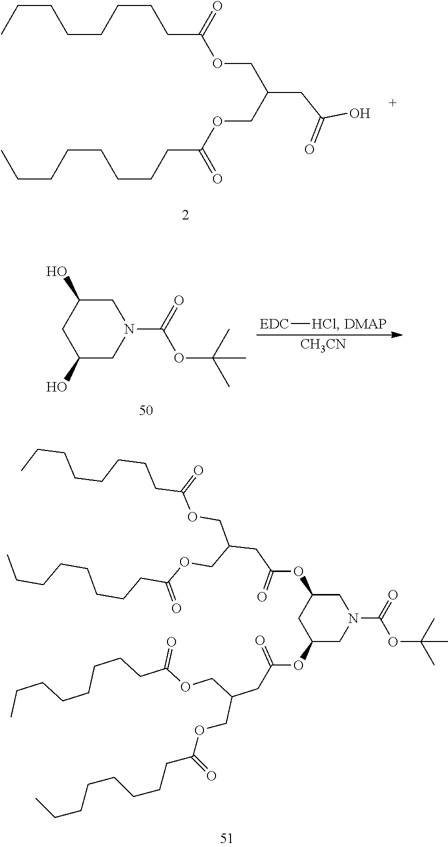

To a solution of 50 (2.50 g, 11.50 mmol) in acetonitrile (200 mL), at room temperature under nitrogen, was added in order 2 (10.00 g, 24.12 mmol), DMAP (1.40 g, 11.50 mmol), and EDC-HCl (5.53 g, 28.85 mmol). The resulting solution was allowed to stir for 18 h at room temperature, then was concentrated in vacuo. The residue was treated with n-heptane (500 mL) and MeOH/H₂O (150 mL, 4:1). The n-heptane phase was separated, washed with MeOH/H₂O (2×150 mL, 4:1) and then dried over Na₂SO₄. The mixture was filtered through a sintered glass funnel, the filter cake was rinsed with n-heptane (50 mL) and the combined filtrates were concentrated in vacuo to afford crude 51 as a viscous, yellow oil. Crude 51 was dissolved in acetonitrile (30 mL) and purified by reverse phase preparative HPLC (Column: XB Phenyl, 50×250 mm, 10 μm; Mobile Phase A: water (0.1% TFA), Mobile Phase B: acetonitrile; Flow rate: 90 mL/min; Gradient: 60% B to 95% B in 10 min, 50% collected; Wave Length: 200 nM). Qualified fractions eluted at 95% B and were concentrated in vacuo. The residue was extracted with n-heptane (500 mL). The n-heptane phase was then extracted with MeOH/H₂O (4:1, 200 mL), H₂O (200 mL), and dried over Na₂SO₄. Filtration and concentration in vacuo gave 51 (10.50 g, 10.39 mmol, 90% yield) as a pale yellow, viscous, oil.

¹H-NMR (300 MHz, CDCl₃): δ=5.30 (brm, 1H), 3.98-4.42 (8H), 3.73 (brm, 1H), 3.42 (brm, 1H), 2.56 (m, 2H), 2.42 (m, 4H), 2.20-2.37 (12H), 2.00 (m, 1H), 1.53-1.68 (8H), 1.49 (s, 9H), 1.20-1.38 (40H), 0.88 (m, 12H); LCMS: RT 2.004, Calcd. for C₅₆H₉₉NO₁₄+Na⁺ 1032.70. Found 1032.60.

### Example 53: Rel-(3R,5S)-3,5-bis((4-(Nonanoyloxy)-3 ((nonanoyloxy)methyl) butanoyl)oxy)piperidin-1-ium trfluoroacetate (52)

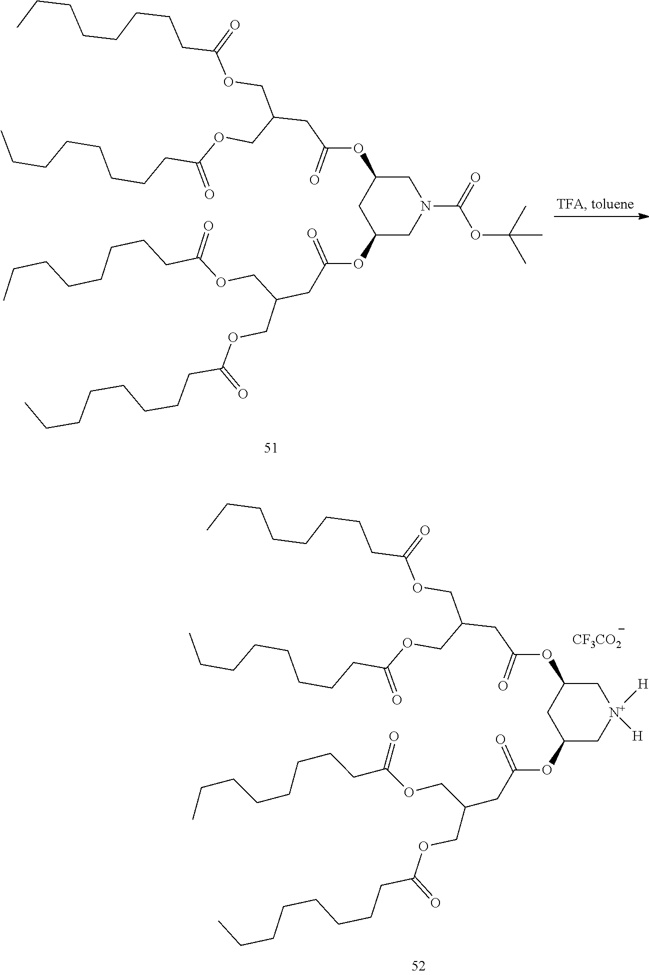

To a solution of 51 (10.50 g, 10.30 mmol) in toluene (47 mL), at room temperature under nitrogen, was added TFA (16 mL, 23.84 g, 0.21 mol) over a period of 10 minutes. The mixture was stirred for 18 h at room temperature, then was diluted with n-heptane (105 mL). The resulting solution was washed with 10% aq. K₂HPO₄ (200 mL), MeOH/H₂O (5:1, 2×100 mL), brine (200 mL), and dried (Na₂SO₄). Filtration and concentration in vacuo afforded crude 52 (9.80 g, 9.57 mmol, 89% HPLC purity, 73% yield) as a pale yellow, viscous, oil.

¹H-NMR (300 MHz, CDCl₃): δ=5.38 (brm, 1H) 4.40 (brm, 2H), 3.90-4.20 (9H), 3.49-3.62 (2H), 2.40-2.70 (7H), 2.31 (m, 8H), 2.07 (m, 1H), 1.56 (m, 8H), 1.19-1.37 (40H), 0.88 (m, 12H); LCMS: RT 1.610, Calcd. for C₅₁H₉₁NO₁₂+Na⁺ 932.64. Found 932.60.

### Example 54: (((Re-(3R,5S)-1-(1H-Imidazole-1-carbonyl)piperidine-3,5-diyl)bis(oxy))bis(2-oxoethane-2,1-diyl))bis(propane-2,1,3-triyl) tetranonanoate (53)

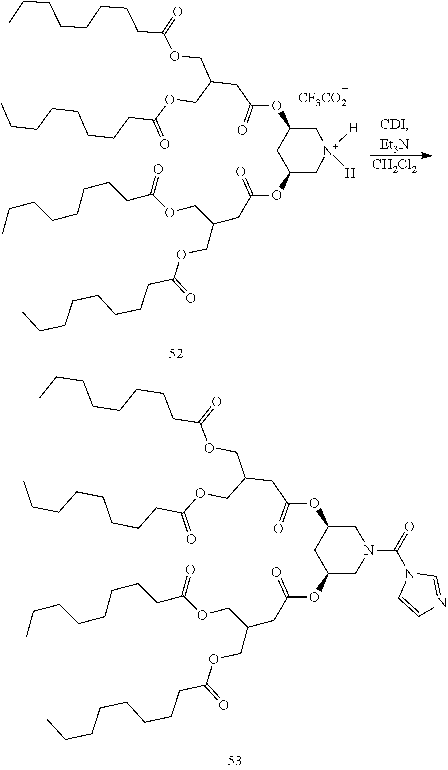

To a solution of 52 (9.80 g, 9.57 mmol) in CH₂Cl₂ (200 mL), cooled in an ice-water bath under nitrogen, was added CDI (15.75 g, 97.13 mmol) followed by Et₃N (4.84 g, 47.9 mmol). The mixture was stirred for 1 hour at 0° C., then was warmed to room temperature and stirred for 18 hours. The solution was washed with 0.5M aq. HCl (2×200 mL), brine (200 mL), and dried (Na₂SO₄). Filtration, and concentration in vacuo afforded crude 53 which was dissolved in n-heptane (400 mL), and the resulting solution was washed with MeOH/H₂O (5:1, 2×100 mL), and the organic phase was dried over Na₂SO₄. Filtration and concentration in vacuo afforded crude 53 (9.30 g, 9.26 mmol, 92% HPLC purity, 96% yield) as a pale yellow oil.

¹H-NMR (300 MHz, CDCl₃): δ=8.06 (brs, 1H), 7.35 (brs, 1H), 7.11 (brs, 1H), 5.27 (m, 1H), 4.70 (m, 1H), 3.90-4.37 (12H) 2.37-2.57 (7H), 2.29 (m, 8H), 2.03 (m, 1H), 1.62 (m, 8H), 1.20-1.37 (40H), 0.88 (m, 12H); LCMS: RT 1.746, Calcd. for C₅₅H₉₃N₃O₁₃+H⁺ 1004.67. Found 1004.60.

### Example 55: (((Rel-(3R,5S)-1-((2-(dimethylamino)ethoxy)carbonyl)piperidine-3,5-diyl)bis(oxy))bis(2-oxoethane-2,1-diyl))bis(propane-2,1,3-triyl) tetranonanoate (CICL-238)

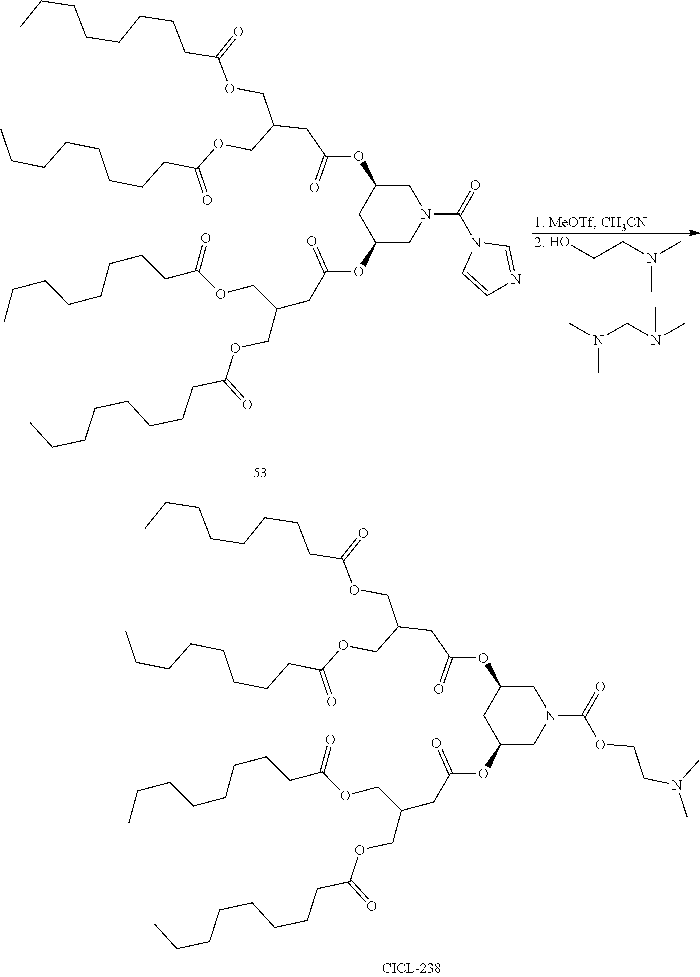

To a solution of 53 (6.00 g, 5.97 mmol) in CH₃CN (60 mL), cooled in an ice-water bath under nitrogen, was added MeOTf (1.18 g, 7.19 mmol) over a period of 5 minutes. The mixture was allowed to stir for 2 hours after the addition was complete, then tetramethyl-ethylenediamine (2.10 g, 18.1 mmol) and dimethyamino-ethanol (0.798 g, 8.95 mmol) were added in order. The mixture was stirred for 1 hour at 0° C. then was warmed to room temperature and stirred for an additional 18 hours. The solvent was removed in vacuo and the residue was dissolved in n-heptane (500 mL) and the resulting solution was washed with MeOH/H₂O (5:1, 2×150 mL), and dried over Na₂SO₄. Filtration and concentration in vacuo afforded crude CICL-238 which was purified by reverse phase preparative HPLC (Column: XB Phenyl, 50×250 mm, 10 μm; Mobile Phase A: water (0.1% TFA), Mobile Phase B: acetonitrile; Flow rate: 90 mL/min; Gradient: 50% B to 90% B in 10 min, 90% collected; Wave Length: 200 nM). Qualified fractions were pooled, and the acetonitrile was removed in vacuo to give CICL-238 which was dissolved in n-heptane (400 mL), and the aqueous phase treated at pH 8 with saturated aq. Na₂CO₃. The organic phase was separated, washed with MeOH/H₂O (5:1, 2×100 mL), water (200 mL), and dried (Na₂SO₄). Filtration and concentration in vacuo afforded CICL-238 (2.04 g, 1.99 mmol, HPLC purity 97%, 33% yield) as a pale, yellow oil.

¹H-NMR (300 MHz, CDCl₃): δ=5.33 (brm, 1H), 4.01-4.41 (13H), 3.77 (brm, 1H), 3.49 (brd, J=12.6 Hz, 1H), 2.57 (m, 4H), 2.42 (m, 4H), 2.18-2.39 (15H), 2.04 (brm, 1H), 1.62 (m, 8H), 1.18-1.39 (40H), 0.89 (m, 12H); LCMS: RT 0.835, Calcd. for C₅₆H₁₀₀N₂O₁₄+H⁺ 1025.73. Found 1025.60.

### Example 56: 2-(2-(((2S,4S)-1-(4-methylpiperazine-1-carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate (CICL-239)

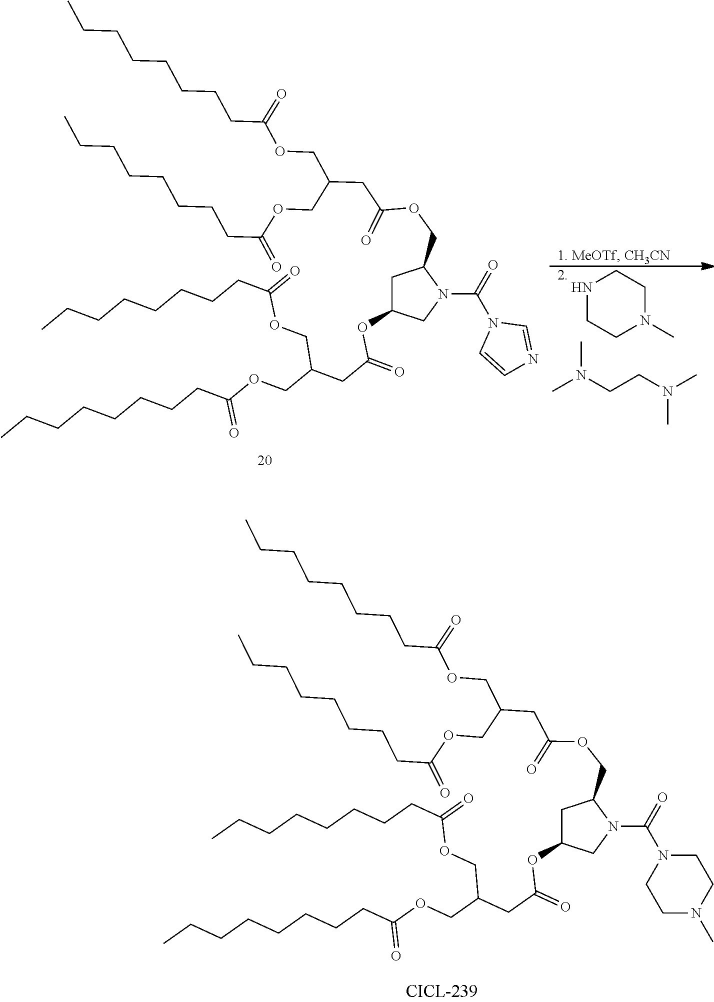

To a solution of 20 (6.00 g, 5.97 mmol), in acetonitrile (60 mL) cooled in an ice-water bath under nitrogen, was added MeOTf (1.18 g, 7.19 mmol) in one portion. The solution was allowed to stir for 2 hours at 0° C., then tetramethylethylenediamine (2.10 g, 18.1 mmol) and N-methylpiperazine (0.90 g, 9.00 mmol) were added in order. The mixture was stirred for 1 hour at 0° C., then warmed to room temperature and stirring was continued for an additional 18 hours. The solvent was removed in vacuo, the residue was dissolved in n-heptane (500 mL), and the resulting solution was washed with MeOH/H₂O (5:1, 2×150 mL), then the organic phase was dried over Na₂SO₄. Filtration and concentration in vacuo provided crude CICL-239, which was purified by reverse phase preparative HPLC (Column: XB Phenyl, 50×250 mm, 10 μm; Mobile Phase A: water (0.1% TFA), Mobile Phase B: acetonitrile; Flow rate: 90 mL/min; Gradient: 50% B to 90% B in 10 min, 90% collected; Wave Length: 200 nM). Qualified fractions were pooled, and the acetonitrile was removed in vacuo to give CICL-239 which was dissolved in n-heptane (400 mL), the aqueous phase was treated at pH 8 with saturated aq. Na₂CO₃. The organic phase was separated, washed with MeOH/H₂O (5:1, 2×100 mL), water (200 mL), and dried (Na₂SO₄). Filtration and concentration in vacuo afforded CICL-239 (2.06 g, 1.99 mmol, HPLC purity 96%, 33% yield) as a pale, yellow oil.

¹H-NMR (300 MHz, CDCl₃): δ=5.13 (m, 1H), 4.52 (m, 1H), 4.04-4.17 (10H), 3.72 (m, 1H), 3.14-3.47 (5H), 2.55 (m, 2H), 2.24-2.50 (20H), 1.81 (m, 1H), 1.63 (m, 8H), 1.18-1.39 (40H), 0.89 (m, 12H); LCMS: RT 1.582, Calcd. for C₅₇H₁₀₁N₃O₁₃+H⁺ 1036.74. Found 1036.60.

### Example 57: Synthesis of ((((Rel-(2R,5S)-1-((2-dimethylamino)ethoxy)carbonyl)pyrrolidine-2,5-diyl)bis(methylene))bis(oxy))bis(2-oxoethane-2,1-diyl))bis(propane-2,1,3-triyl) tetranonanoate (CICL-242)

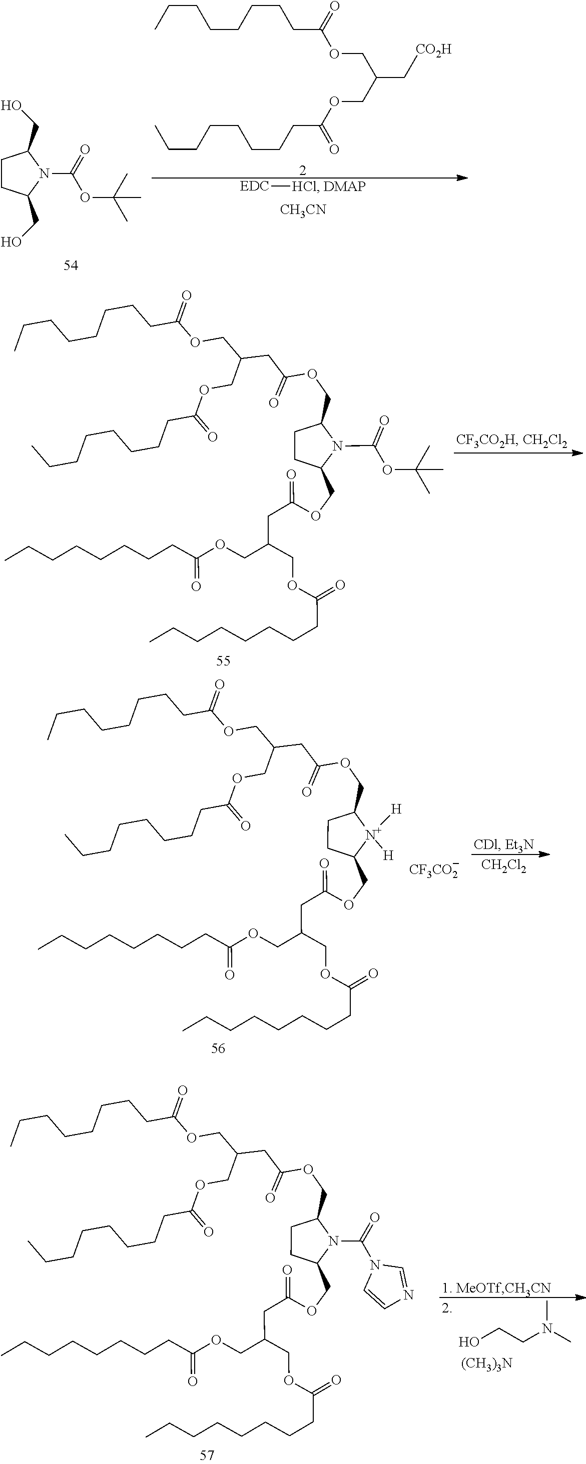

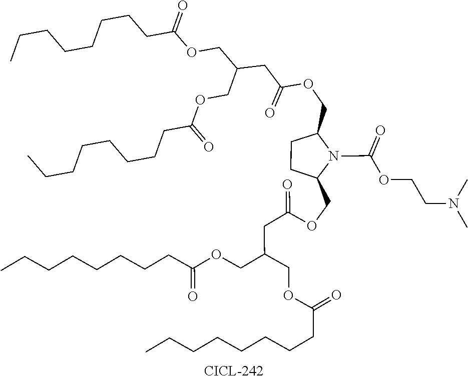 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCN(C)C]

tert-Butyl Rel-(2R,5S)-2,5-bis(hydroxymethyl)pyrrolidine-1-carboxylate 54 (Synthesis 2013, 45, 2966-2970) is coupled with 2 utilizing EDC-HCl and DMAP in CH₃CN to give 55. Deprotection of the BOC-amine in 55 with CF₃CO₂H in CH₂Cl₂, provides ammonium trifluoroacetate salt 56. Treatment of 56 with carbonyl-di-imidazole and Et₃N, in CH₂Cl₂, then gives acylimidazole 57. Alkylation of 57 with MeOTf in CH₃CN, followed by the reaction of the derived acylimidazolium salt with 2-dimethylaminoethanol and (CH₃)₃N results in CICL-242.

### Example 58: Synthesis of (((((2S,5S)-1-((2-(dimethylamino)ethoxy)carbonyl)pyrrolidine-2,5-diyl)bis(methylene))bis(oxy))bis(2-oxoethane-2,1-diyl))bis(propane-2,1,3-triyl) tetranonanoate (CICL-243)

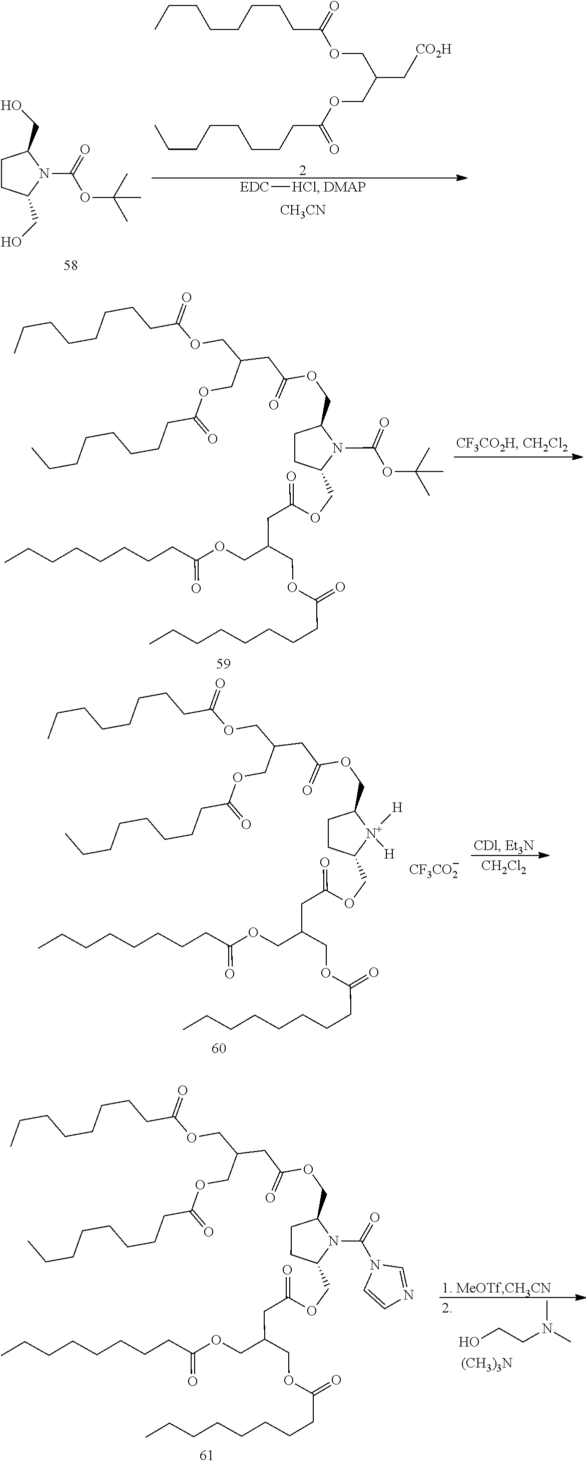

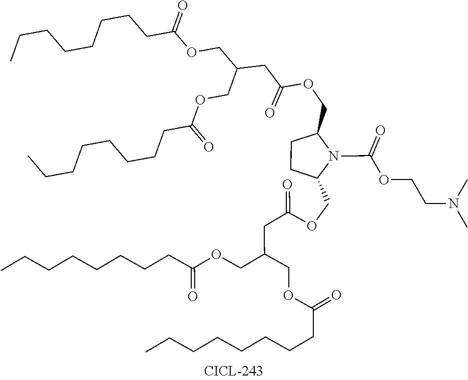 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCN(C)C]

tert-Butyl (2S,5S)-2,5-bis(hydroxymethyl)pyrrolidine-1-carboxylate 58 (WO2023141570, Tetrahedron Lett. 1989, 29, 3805) is coupled with 2 utilizing EDC-HCl and DMAP in CH₃CN to give 59. Deprotection of the BOC-amine in 59 with CF₃CO₂H in CH₂Cl₂, provides ammonium trifluoroacetate salt 60. Treatment of 60 with carbonyl-di-imidazole and Et₃N in CH₂Cl₂, then gives acylimizadole 61. Alkylation of 61 with MeOTf in CH₃CN, followed by the reaction of the derived acylimidazolium salt with 2-dimethylaminoethanol and (CH₃)₃N results in CICL-243.

### Example 59: Synthesis of (((Rel-((2S,6R)-1-((2-(dimethylamino)ethoxy)carbonyl)piperidine-2,6-diyl)bis(methylene))bis(oxy))bis(2-oxoethane-2,1-diyl))bis(propane-2,1,3-triyl) tetranonanoate (CICL-244)

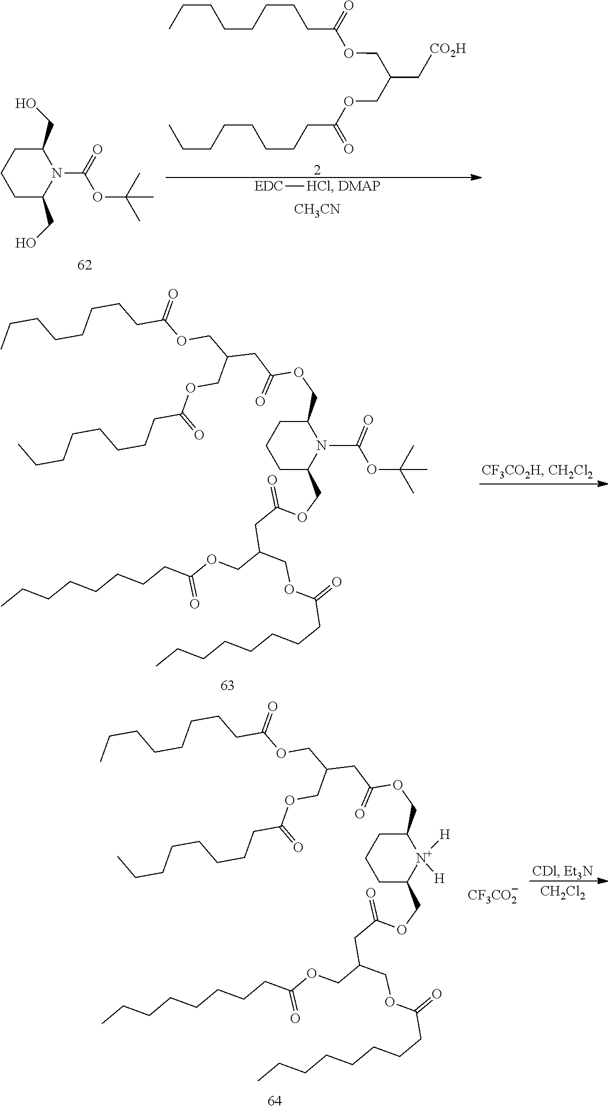

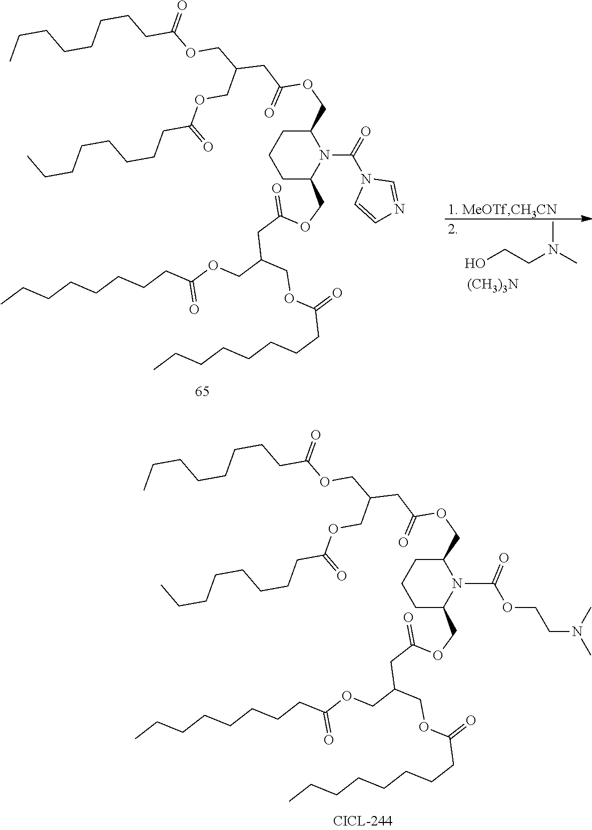

tert-Butyl Rel-(2S,6R)-2,6-bis(hydroxymethyl)piperidine-1-carboxylate 62 (Bioorg. Med. Chem. Lett. 2010, 20, 3584) is coupled with 2 utilizing EDC-HCl and DMAP in CH₃CN to give 63. Deprotection of the BOC-amine in 63 with CF₃CO₂H in CH₂Cl₂, provides ammonium trifluoroacetate salt 64. Treatment of 64 with carbonyl-di-imidazole and Et₃N in CH₂Cl₂, gives acylimizadole 65. Alkylation of 65 with MeOTf in CH₃CN, followed by the reaction of the derived acylimidazolium salt with 2-dimethylaminoethanol and (CH₃)₃N results in CICL-244.

### Example 60: Synthesis of ((((Rel-(2S,6S)-1-((2-(dimethylamino)ethoxy)carbonyl)piperidine-2,6-diyl)bis(methylene))bis(oxy))bis(2-oxoethane-2,1-diyl))bis(propane-2,1,3-triyl) tetranonanoate (CICL-245)

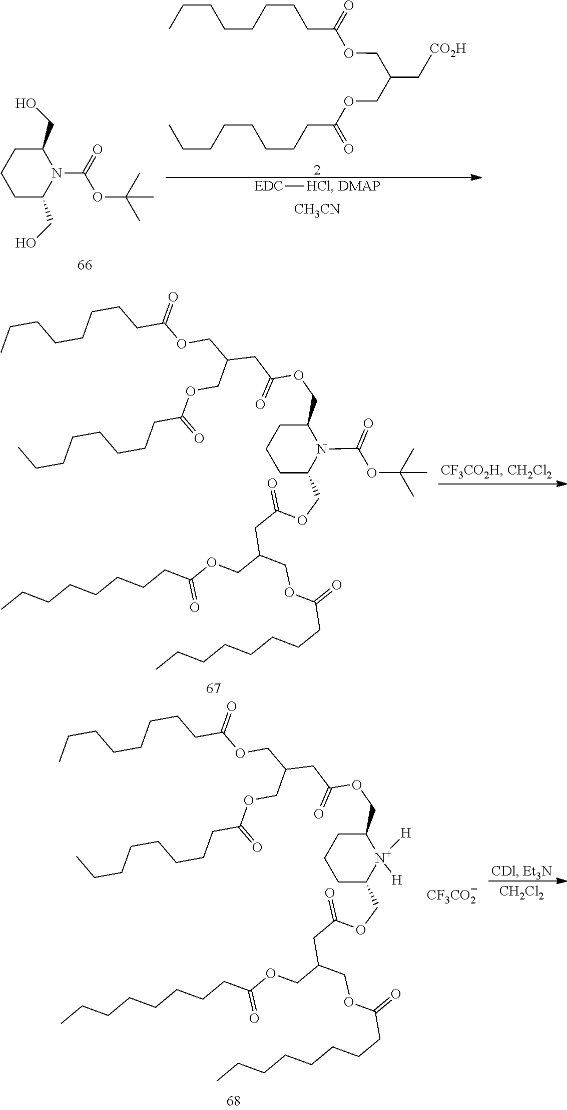

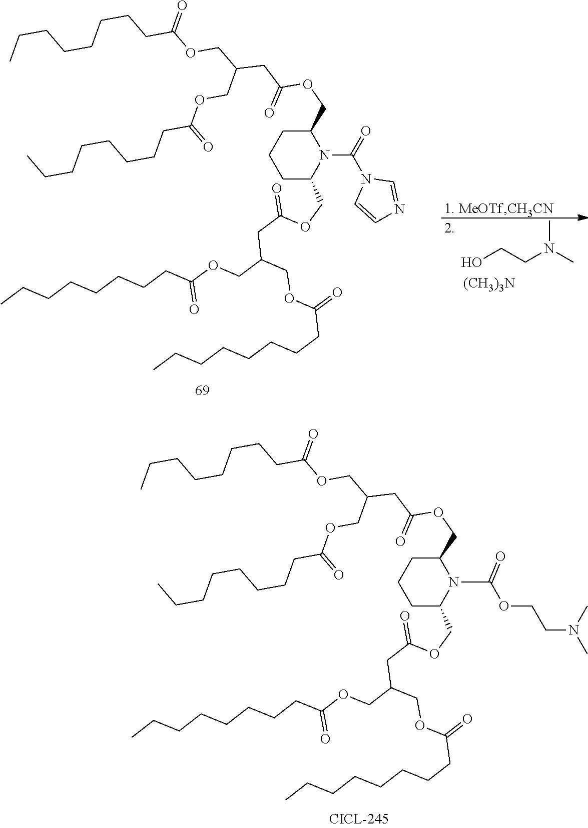

tert-Butyl Rel-(2S,6S)-2,6-bis(hydroxymethyl)piperidine-1-carboxylate 66 (Tetrahedron Lett. 1989, 30, 6077) is coupled with 2 utilizing EDC-HCl and DMAP in CH₃CN to give 67. Deprotection of the BOC-amine in 67 with CF₃CO₂H in CH₂Cl₂, provides ammonium trifluoroacetate salt 68. Treatment of 68 with carbonyl-di-imidazole and Et₃N in CH₂Cl₂, then gives acylimizadole 69. Alkylation of 692 with MeOTf in CH₃CN, followed by the reaction of the derived acylimidazolium salt with 2-dimethylaminoethanol and (CH₃)₃N results in CICL-245.

### Example 61: Synthesis of ((((Rel-(2R,7S)-1-((2-(dimethylamino)ethoxy)carbonyl)-2,3,6,7-tetrahydro-1H-azepine-2,7-diyl)bis(methylene))bis(oxy))bis(2-oxoethane-2,1-diyl))bis(propane-2,1,3-triyl) tetranonanoate (CICL-246)

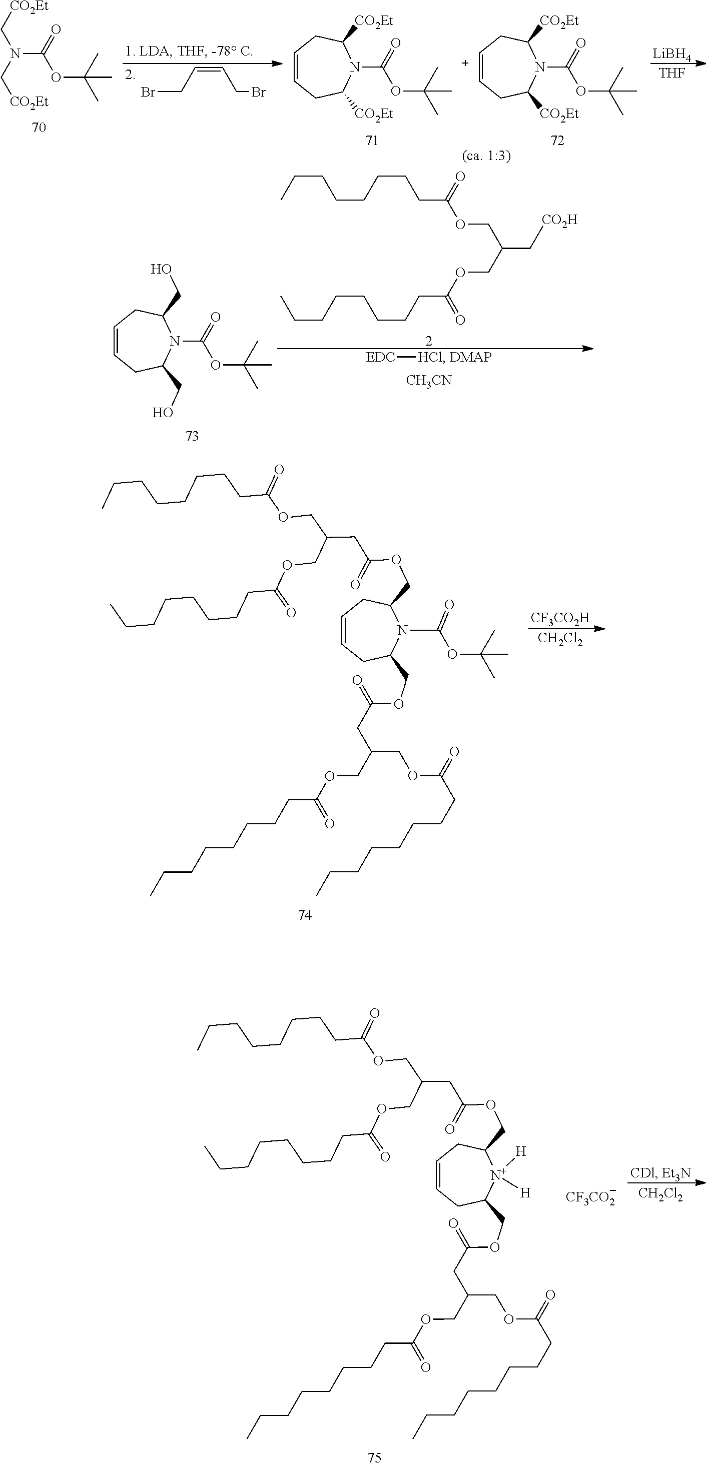

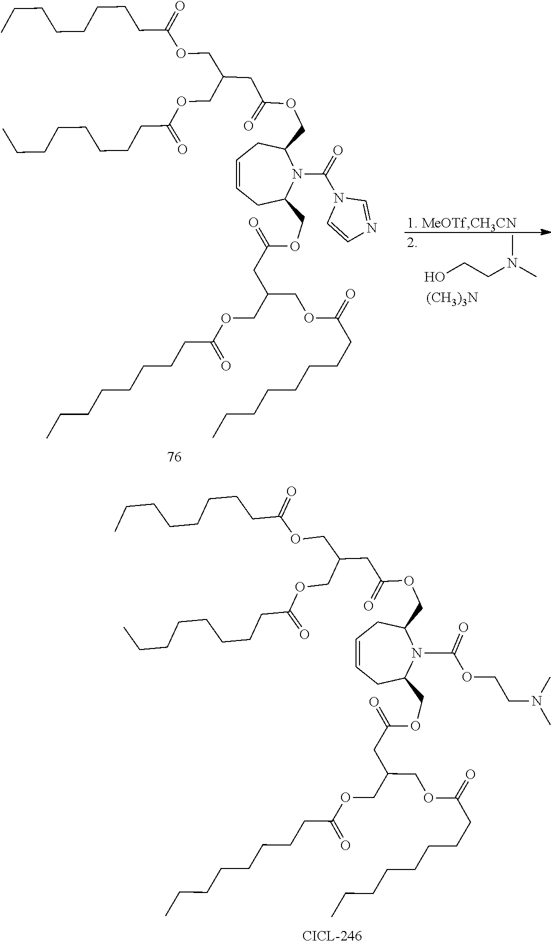

As described in Synlett 1994, 1093, alkylation of the dianion of 70 with Z-1-4-dibromo-2-butene (J. Org. Chem. 2020, 85, 5787) leads to a mixture of diesters 71 and 72 in approximately a 1:3 ratio. Separation of the diesters, and reduction of 72 (LiBH₄, THF) leads to diol 73. Diol 73 is coupled with 2 utilizing EDC-HCl and DMAP in CH₃CN to give 74. Deprotection of the BOC-amine in 74 with CF₃CO₂H in CH₂Cl₂, provides ammonium trifluoroacetate salt 75. Treatment of 75 with carbonyl-di-imidazole and Et₃N in CH₂Cl₂, then gives acylimizadole 76. Alkylation of 76 with MeOTf in CH₃CN, followed by the reaction of the derived acylimidazolium salt with 2-dimethylaminoethanol and (CH₃)₃N results in CICL-246.

### Example 62: Synthesis of ((((Re-(2S,7S)-1-((2-(dimethylamino)ethoxy)carbonyl)-2,3,6,7-tetrahydro-1H-azepine-2,7-diyl)bis(methylene))bis(oxy))bis(2-oxoethane-2,1-diyl))bis(propane-2,1,3-triyl) tetranonanoate (CICL-247)

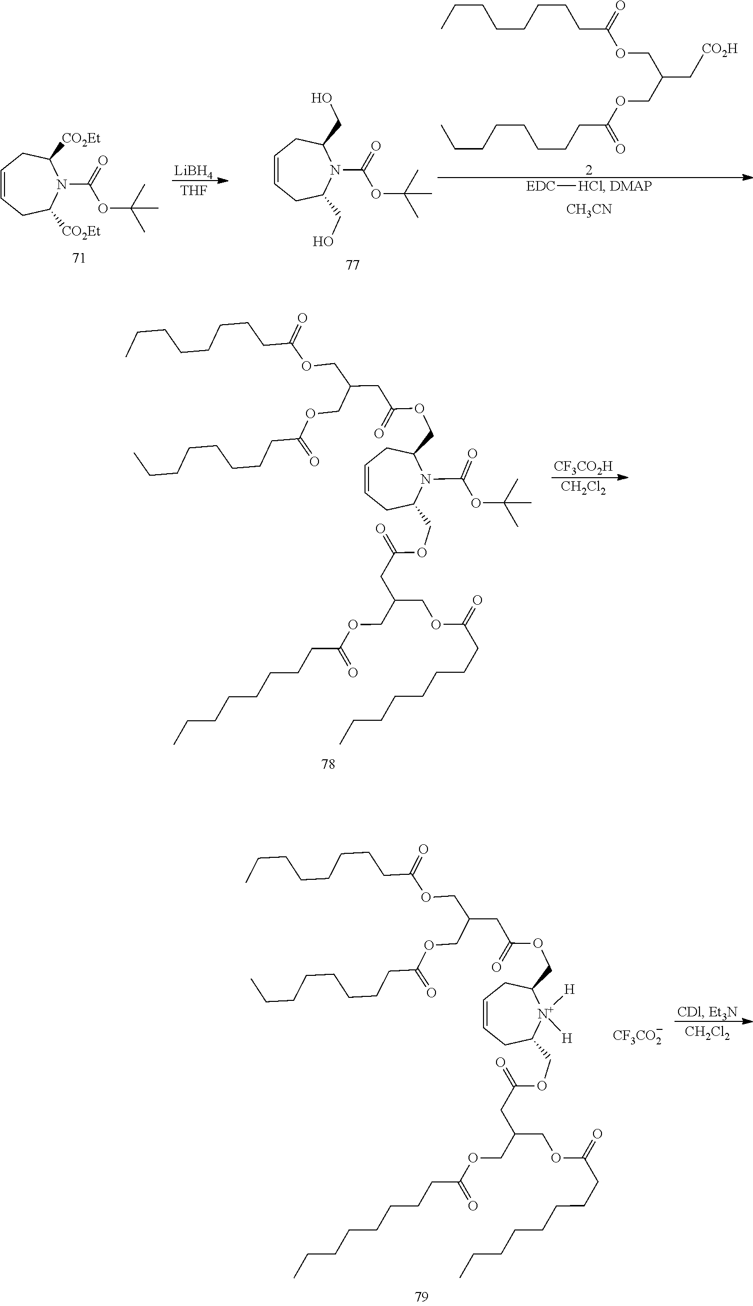

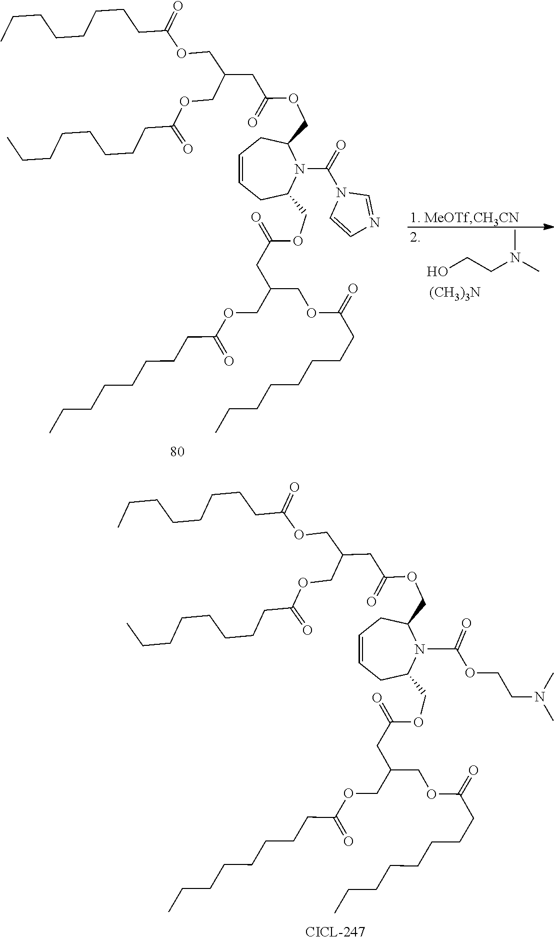

Reduction of diester 71, prepared as described in Example 62, with LiBH₄ in THF, leads to diol 77. Diol 77 is coupled with 2 utilizing EDC-HCl and DMAP in CH₃CN to give 78. Deprotection of the BOC-amine in 78 with CF₃CO₂H in CH₂Cl₂, provides ammonium trifluoroacetate salt 79. Treatment of 79 with carbonyl-di-imidazole and Et₃N in CH₂Cl₂, then gives acylimizadole 80. Alkylation of 80 with MeOTf in CH₃CN, followed by the reaction of the derived acylimidazolium salt with 2-dimethylaminoethanol and (CH₃)₃N results in CICL-247.

### Example 63: Synthesis of ((((Rel-(2S,7R)-1-((2-(dimethylamino)ethoxy)carbonyl)azepane-2,7-diyl)bis(methylene))bis(oxy))bis(2-oxoethane-2,1-diyl))bis(propane-2,1,3-triyl) tetranonanoate (CICL-248)

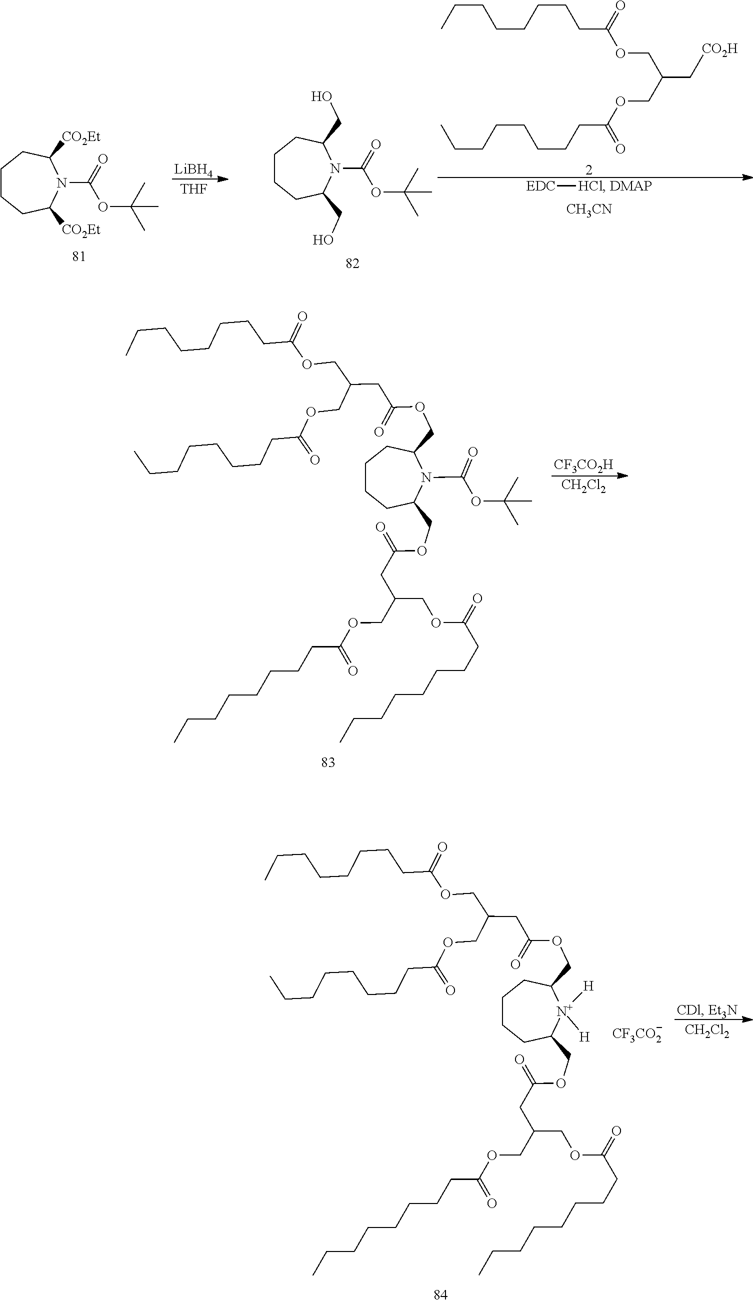

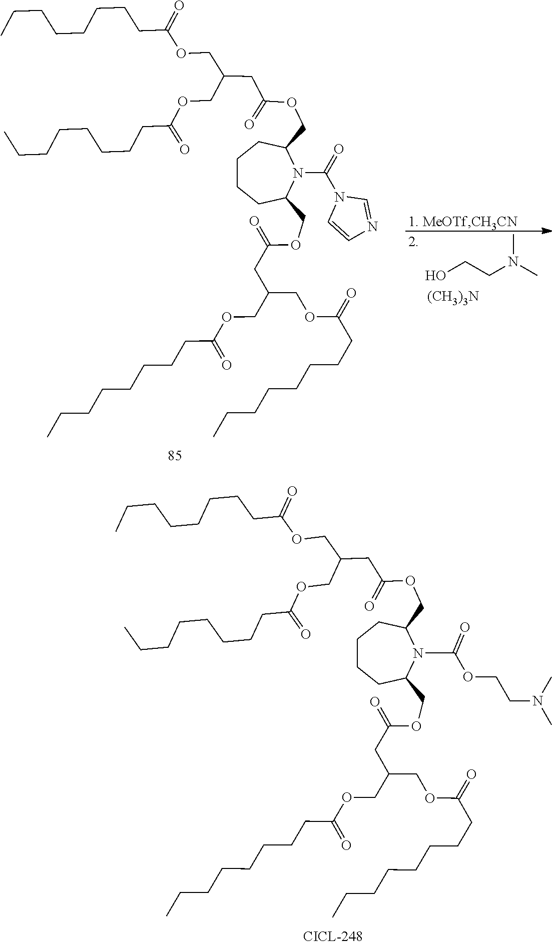

1-(tert-Butyl) 2,7-diethyl Rel-(2S,7R)-azepane-1,2,7-tricarboxylate 81 (Synlett 1994, 1023) is reduced with LiBH₄ in THF, to give diol 82. Diol 82 is coupled with 2 utilizing EDC-HCl and DMAP in CH₃CN to give 83. Deprotection of the BOC-amine in 83 with CF₃CO₂H in CH₂Cl₂, provides ammonium trifluoroacetate salt 84. Treatment of 84 with carbonyl-di-imidazole and Et₃N in CH₂Cl₂, then gives acylimizadole 85. Alkylation of 85 with MeOTf in CH₃CN, followed by the reaction of the derived acylimidazolium salt with 2-dimethylaminoethanol and (CH₃)₃N results in CICL-248.

### Example 64: Synthesis of ((((Re-(2S,7S)-1-((2-(dimethylamino)ethoxy)carbonyl)azepane-2,7-diyl)bis(methylene))bis(oxy))bis(2-oxoethane-2,1-diyl))bis(propane-2,1,3-triyl) tetranonanoate (CICL-249)

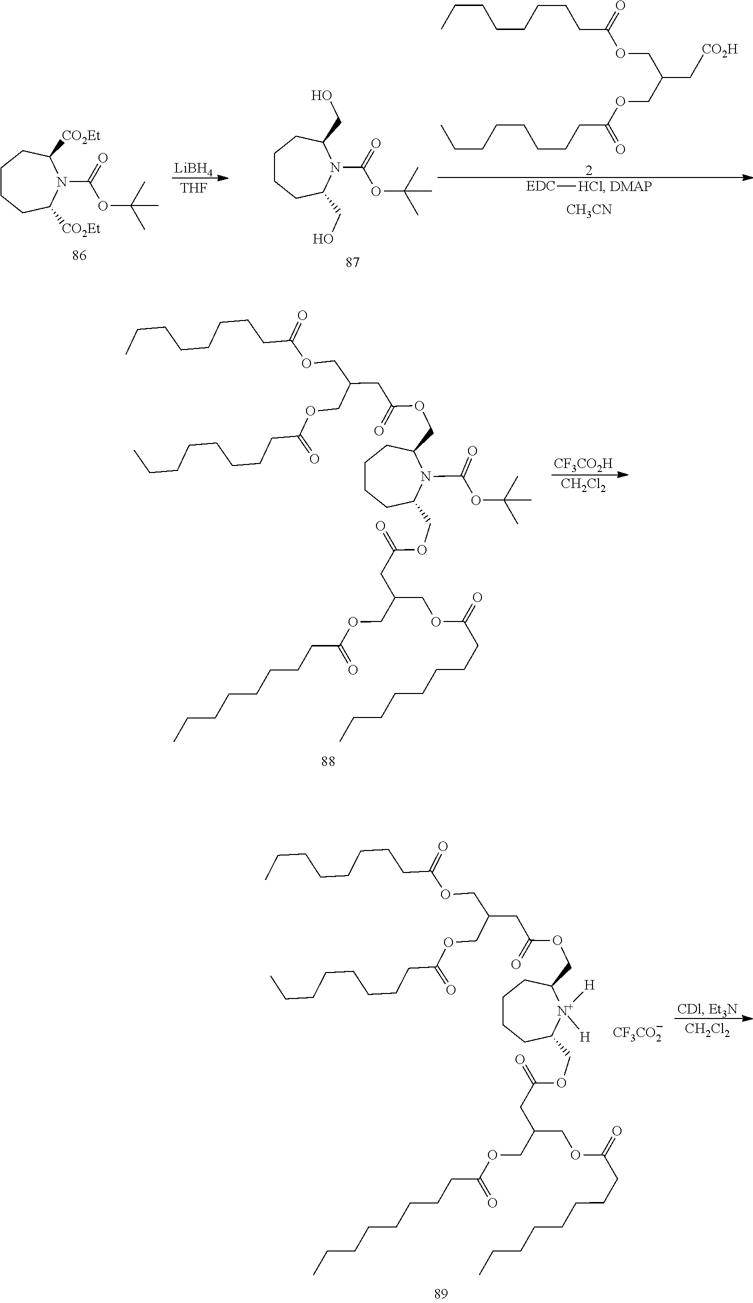

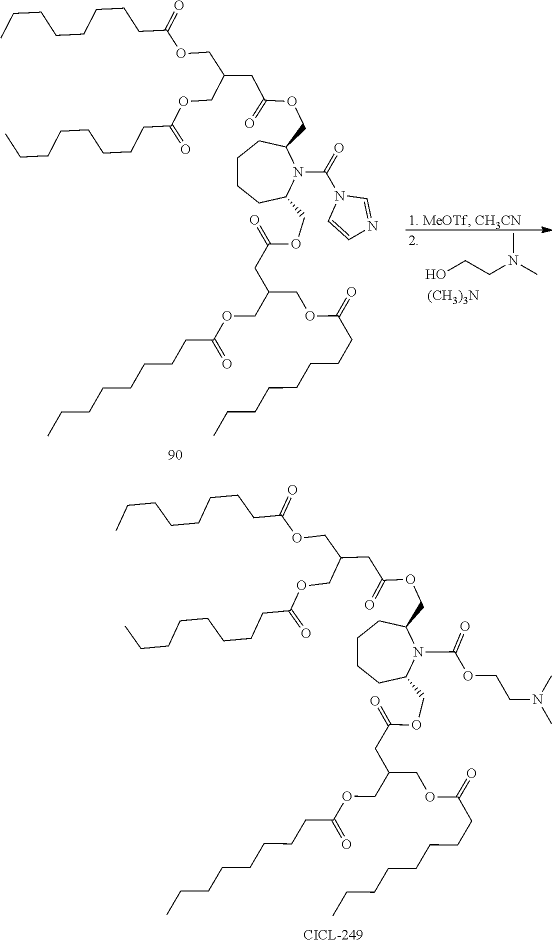

1-(tert-Butyl) 2,7-diethyl Rel-(2S,7S)-azepane-1,2,7-tricarboxylate 86 (Synlett 1994, 1023) is reduced with LiBH₄ in THF, to give diol 87. Diol 87 is coupled with 2 utilizing EDC-HCl and DMAP in CH₃CN to give 88. Deprotection of the BOC-amine in 88 with CF₃CO₂H in CH₂Cl₂, provides ammonium trifluoroacetate salt 89. Treatment of 89 with carbonyl-di-imidazole and Et₃N in CH₂Cl₂, then gives acylimizadole 90. Alkylation of 90 with MeOTf in CH₃CN, followed by the reaction of the derived acylimidazolium salt with 2-dimethylaminoethanol and (CH₃)₃N results in CICL-249.

### Example 65: Synthesis of 2-(2-(((2S,4R)-1-(((1-methylazetidin-3-yl)oxy)carbonyl)-4-((4-(nonanoyloxy)-3-((nonanoyloxy)methyl)butanoyl)oxy)pyrrolidin-2-yl)methoxy)-2-oxoethyl)propane-1,3-diyl dinonanoate: CICL-207-91

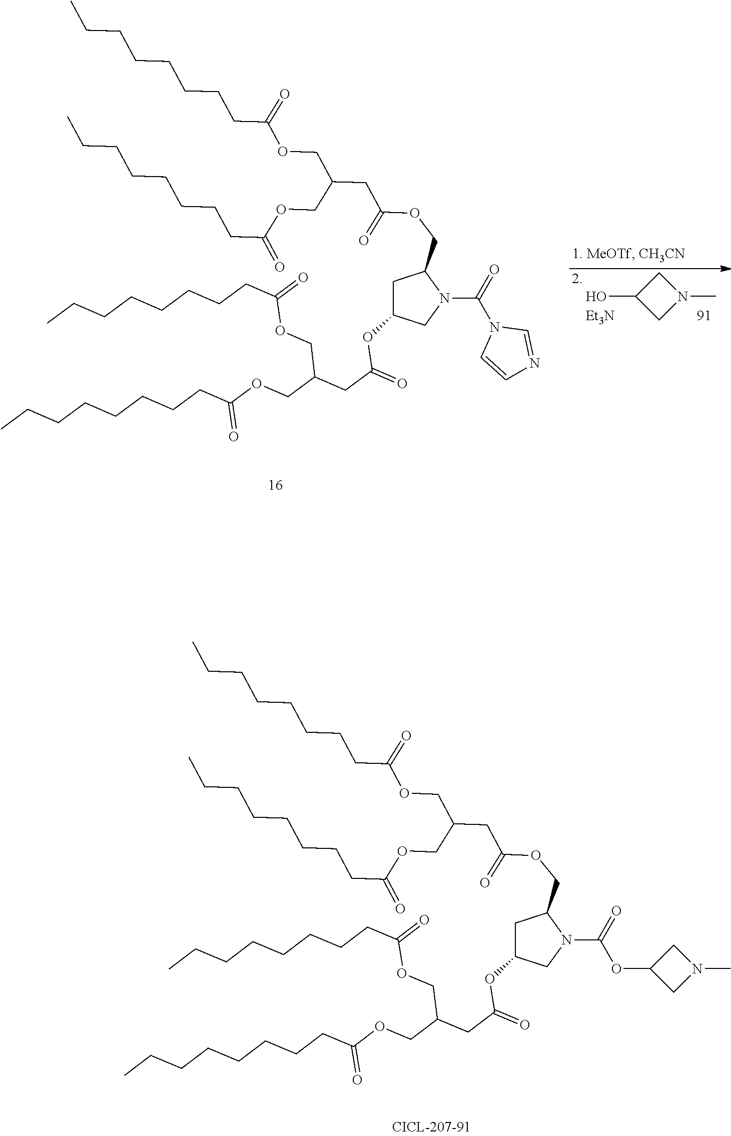

Acylimidazolide 16, when reacted with MeOTf provides an activated acylimidazolium species. The reaction of the activated acylimidazolium species with alcohol 91 (CombiBlocks #JL-5330), in the presence of triethyl amine leads to CICL-207-91.

The numbering paradigm utilized for cyclic-amino-alcohol head groups is based on an ionizable cationic lipid described above that was generated from an acylimidazolide, in this case 16, when it was reacted with 2-dimethylaminoethanol, with the number of the head group-forming alcohol appended, in this case 91. Acylimidazolide 16 was converted to lipid CICL-207 in Example 17.

The utilization of the chemistry described in this Example 65 to generate the active imidazolium species, enables the synthesis of a selection of lipids containing cyclic head groups by substitution of the alcohol 91 (vide supra) with the alcohols appearing in Table 3.

TABLE 3
CICL-207-91 and Substitution of Alcohol 91 with Alcohols 92-107 to Prepare
Lipids CICL-207-92 to CICL-207-107

| Alcohol | Source | Lipid | c-pKa* |
|---|---|---|---|
| 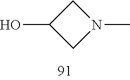 | CombiBlocks #JL-5330 | 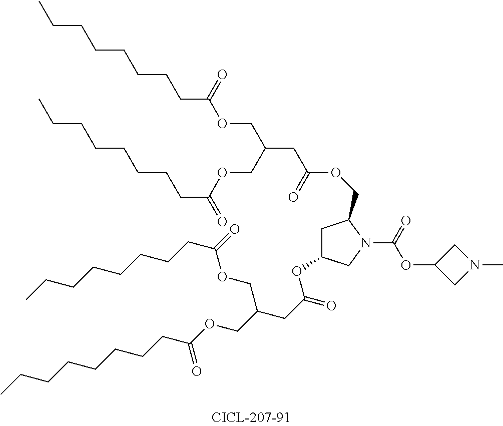 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CN(C)C1] | 8.39 |
| 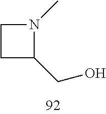 | CombiBlocks #JL-5446 | 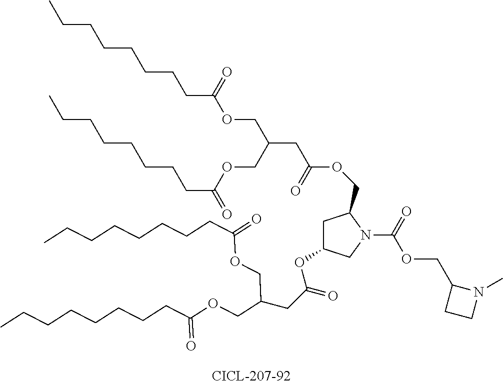 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCN1C] | 8.62 |
| 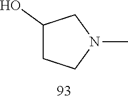 | CombiBlocks #AM-2015 | 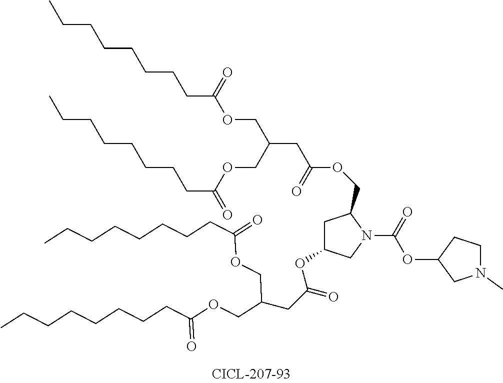 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCN(C)C1] | 8.70 |
| 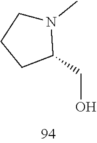 | CombiBlocks #OR-0903 | 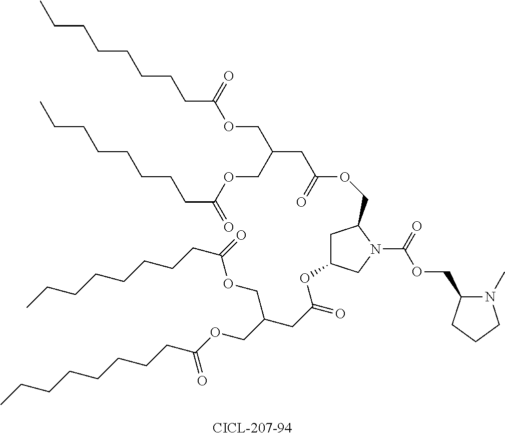 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC[C@@H]1CCCN1C] | 9.47 |
| 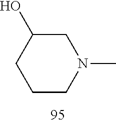 | J. Med. Pharm. Chem. 1960, 2, 107 | 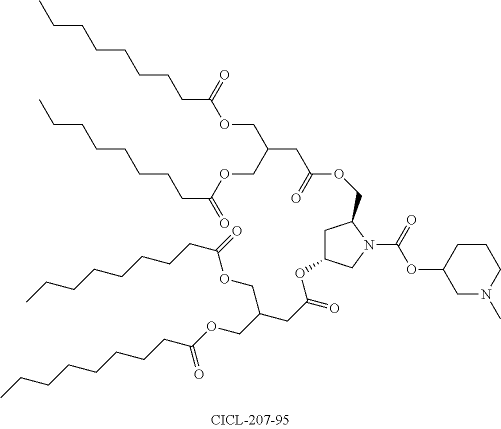 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCCN(C)C1] | 8.83 |
| 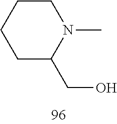 | CombiBlocks #OR-1501 | 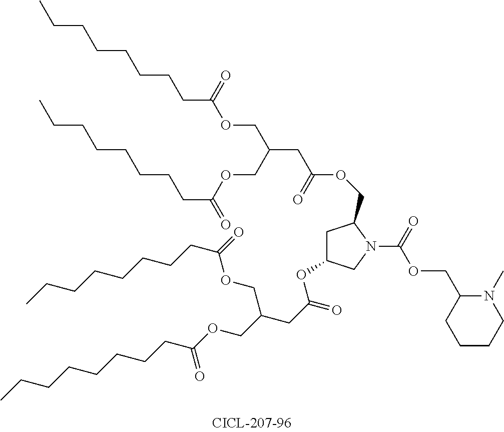 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCCN1C] | 9.24 |
| 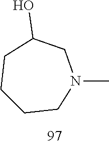 | CombiBlocks #ST-4233 WO2008022979 J. Med. Pharm. Chem. 1960, 2, 107 | 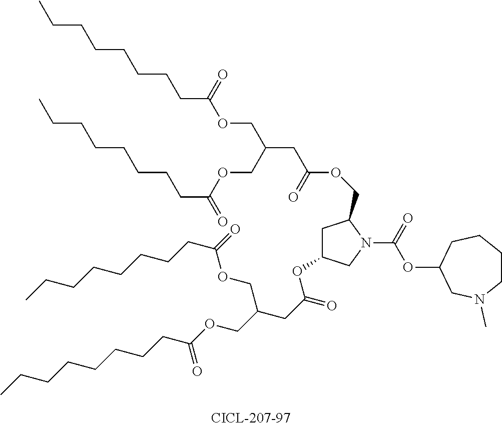 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCCCN(C)C1] | 8.44 |
| 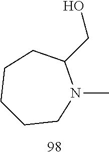 | CombiBlocks #JA-4992 J. Med. Pharm. Chem. 1960, 2, 107 | 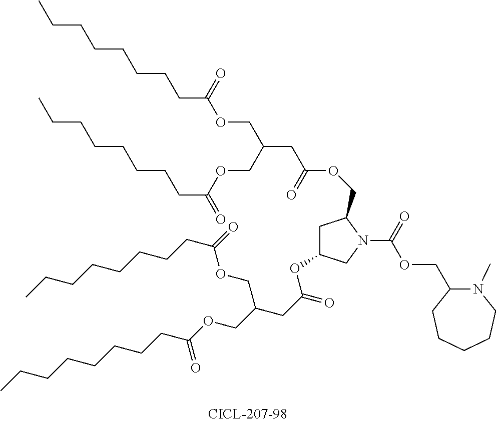 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCCCN1C] | 8.88 |
| 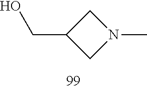 | CombiBlocks #QV-6461 | 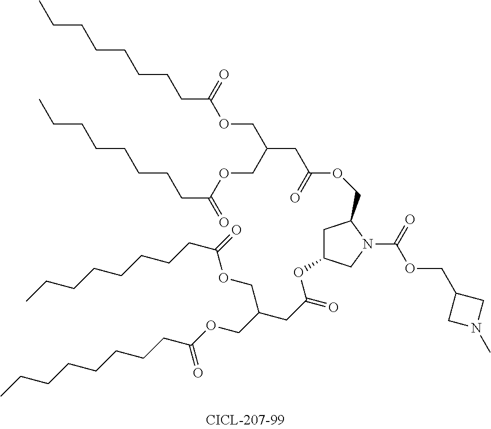 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CN(C)C1] | 9.23 |
| 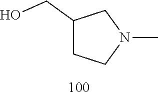 | CombiBlocks #QH-5249 | 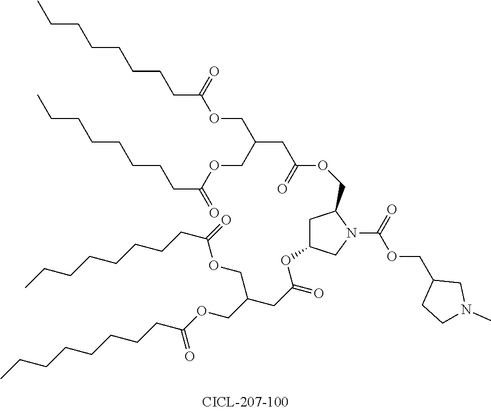 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCN(C)C1] | 9.83 |
| 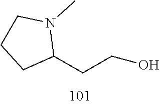 | CombiBlocks #ST-1090 | 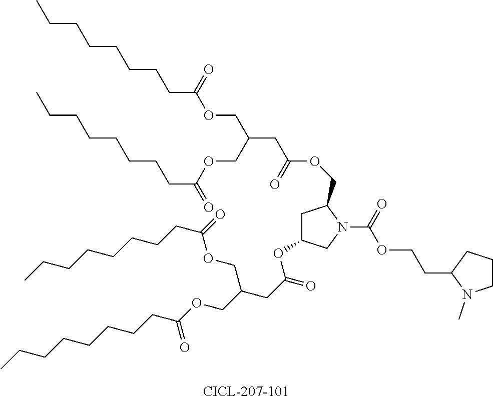 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCC1CCCN1C] | 10.28 |
| 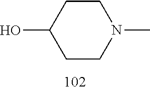 | CombiBlocks #AM-2246 | 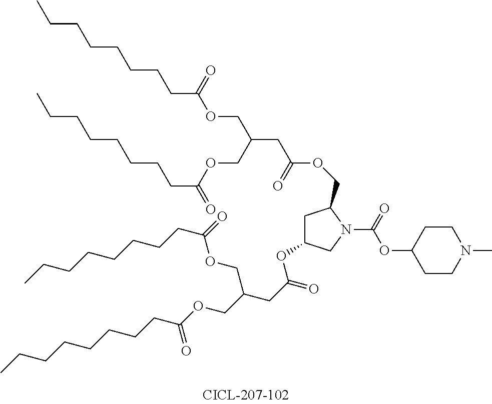 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCN(C)CC1] | 8.03 |
| 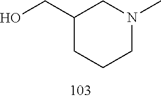 | CombiBlocks #OR-1504 | 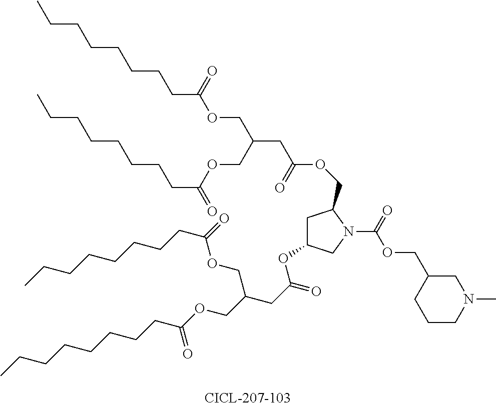 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCN(C)C1] | 9.20 |
| 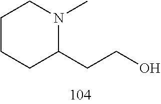 | CombiBlocks #QZ-3821 | 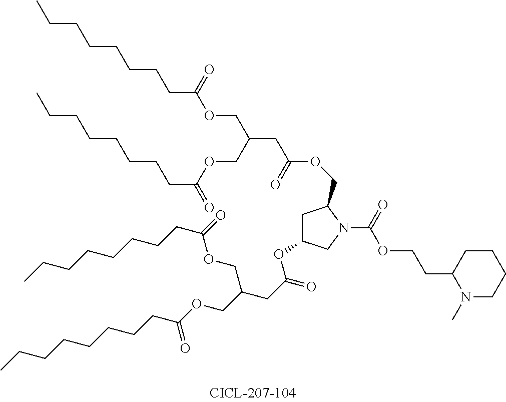 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCC1CCCCN1C] | 9.75 |
| 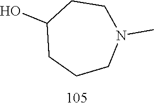 | CombiBlocks #SS-2386 | 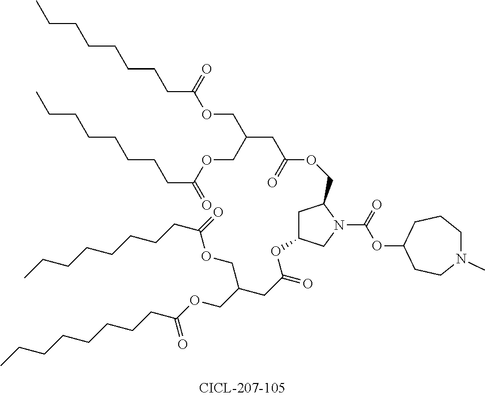 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OC1CCCN(C)CC1] | 8.81 |
| 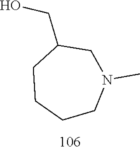 | Enamine #EN300-147890 Med. Pharm. Chem. 1960, 2, 107 | 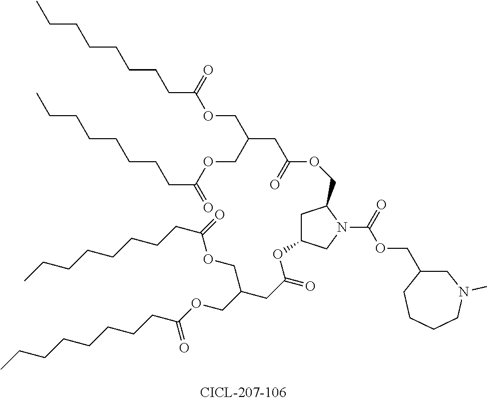 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCC1CCCCN(C)C1] | 9.34 |
| 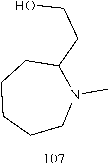 | Enamine #EN300-202111 Med. Pharm. Chem. 1960, 2, 107 | 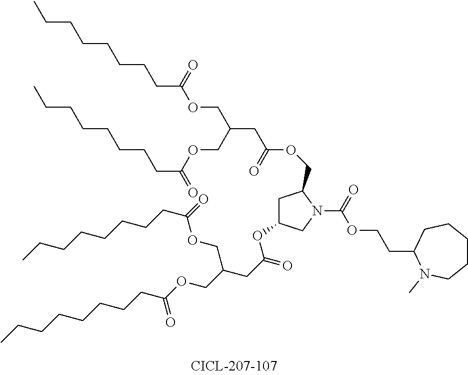 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCC1CCCCCN1C] | 9.52 |

In a similar fashion, utilizing the chemistry of this Example 65, the various constrained core acylimidazolides: 7, 12, 20, 24, 28, 32, 36, 40, 44, 48, 53, 57, 61, 65, 69, 76, 80, 85 and 90 are reacted with alcohols 91-107 to create the congeners having the various cyclic head groups on each of the constrained cores described above. Given a desired range of measured pKa of from 6-7, a restriction of the range of calculated pKa (c-pKa) is likely needed to achieve that target measured pKa range. The alcohols which are appended to acylimidazolide 16 to provide lipids CICL-207-91 to CICL-207-107, which result in a calculated pKa (c-pKa) of ca. 8 to 9 (Table 3), are utilized to form lipids with the constrained core acylimidazolides 7, 12, 20, 24, 28, 32, 36, 40, 44, 48, 53, 57, 61, 65, 69, 76, 80, 85 and 90. The alcohols thus selected are: 91, 92, 93, 95, 97, 98,102, and 105.

To obtain lipids conferring advantageous in vivo transfection performance on tLNPs incorporating the lipid, the alcohols should be selected to alter the measured pKa values for the core imidazolides appended with N,N-dimethylamino ethanol in Table 10 toward the mid-point of the target range of 6-7. To maintain or reduce the basicity of the lipids prepared from acylimidazolide core structure 7 (used in synthesizing CICL-221), alcohols 91, 97, and 102 are selected to generate CICL-221-91, CICL-221-97, and CICL-221-102. Core acylimidazolide 12 (used in synthesizing CICL-222) is combined with alcohols 91, 92, 93, 95, 97, 98, and 105 to maintain or raise measured pKa as described to generate congeners of CICL-222. Core acylimidazolide 20 (used in synthesizing CICL-224) is combined with alcohols 91, 97, and 102 to maintain or reduce measured pKa toward the target range and generate CICL-224-91, CICL-224-97, and CICL-224-102. The combination of core acylimidazolide 24 (used in synthesizing CICL-225) with alcohols 91, 92, 93, 95, 97, and 98 is selected to maintain or raise measured pKa toward the desired target range to generate congeners of CICL-225. Acylimidazolide 28, (used in synthesizing CICL-223) when combined with alcohols 91, 97, and 102, results in lipids (CICL-223-91, CICL-223-97, and CICL-223-102) that approach the target measured pKa values by maintaining or reducing basicity. Acylimidazolide 32 (used in synthesizing CICL-216) is combined with alcohols 91, 97, and 102 to afford lipids (CICL-216-91, CICL-216-97, and CICL-216-102) in the target pKa range by maintaining or reducing basicity. Core acylimidazolide 36 (used in synthesizing CICL-215) is combined with alcohols 91, 92, 93, 95, and 97 to create lipids (congeners of CICL-215) with similar basicity and a pKa in the target pKa range. Acylimidazolide 40, (used in synthesizing CICL-220) when combined with alcohols 91, 92, 93, 95, 97, 98, and 105 gives lipids (congeners of CICL-220) with a maintained or raised basicity moving pKa toward the desired target range. Similarly, the combination of acylimidazolide 44 (used in synthesizing CICL-219) with alcohols 91, 92, 93, 95, 97, 98, and 105 provides lipids (congeners of CICL-219) with a maintained or raised basicity moving pKa toward the desired target pKa range. The reaction of acylimidazolide 48 (used in synthesizing CICL-218) with alcohols 94, 96, 99, 103, and 106 provides lipids (congeners of CICL-218) with a maintained or raised basicity moving pKa toward the target pKa range. The combination of acylimidazolide 53 (used in synthesizing CICL-238) with alcohols 91, 97, and 102 leads to lipids (CICL-238-91, CICL-238-97, and CICL-238-102) with maintained or reduced basicity and measured pKa values, within the target zone. The concatenation of acylimidazolide 57 (used in synthesizing CICL-242), which is not associated with a measured pKa value in Table 10, with alcohols 91, 92, 93, 95, 97, 98, 102, and 105 affords lipids (congeners of CICL-242) targeted for measured pKa values within the desired range by having a similar basicity to CICL-207. Similarly, the reaction of acylimidazolides 61, 65, 69, 76, 80, 85, and 90 (used in synthesizing CICL-243, CICL-244, CICL-245, CICL-246, CICL-247, CICL-248, and CICL-249, respectively) with alcohols 91, 92, 93, 95, 97, 98, 102, and 105 provides lipids targeted for measured pKa values within the desired range by having a similar basicity to CICL-207.

Table 4 presents the structures that result from the combination of acylimidazolide core structures: 7, 12, 20, 24, 28, 32, 36, 40, 44, 48, 53, 57, 61, 65, 69, 76, 80, 85 and 90 with the above mentioned alcohols (91, 92, 93, 95, 97, 98, 102, and 105) which are expected to result in lipids with calculated pKa values in the range of 8-9. The numbering convention applied to the structures in Table 4, as described above, is taken from the initial lipid structure created from the listed core acylimidazolide, now combined with the selected alcohols listed above, e.g. 7 leading to CICL-221, when 7 might be combined with alcohol 91, the number appearing in Table 4 is listed as: CICL-221-91.

TABLE 4
Lipids formed from the Combination of Core Acylimidazolides 7, 12, 20, 24, 28, 32,
36, 40, 44, 48, 53, 57, 61, 65, 69, 76, 80, 85 and 90 with Alcohols selected from 91, 92, 93,
94, 95, 97, 98, 99, 102, 103, 105, and 106.

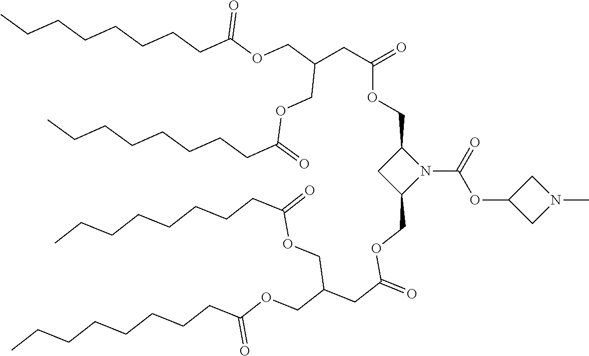 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OC1CN(C)C1]

CICL-221-91

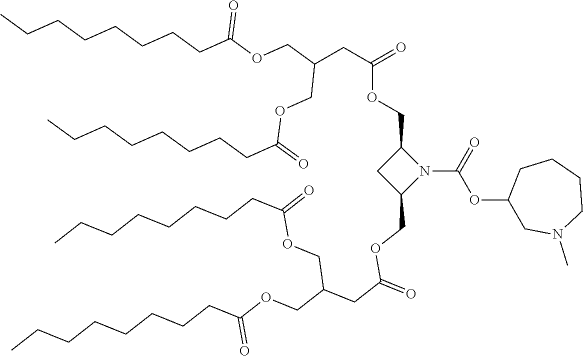 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OC1CCCCN(C)C1]

CICL-221-97

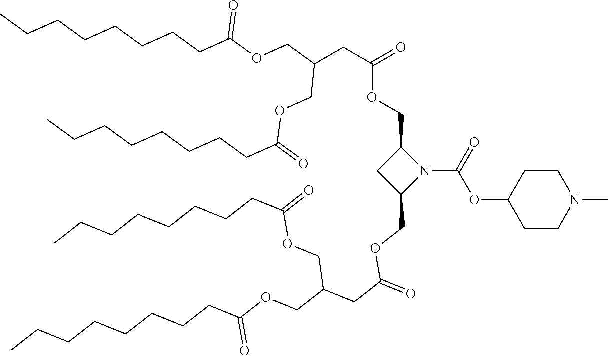 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OC1CCN(C)CC1]

CICL-221-102

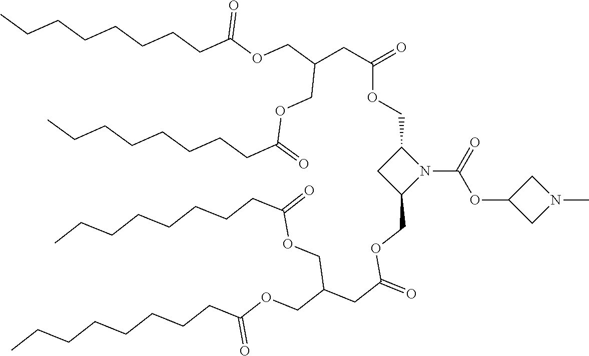 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OC1CN(C)C1]

CICL-222-91

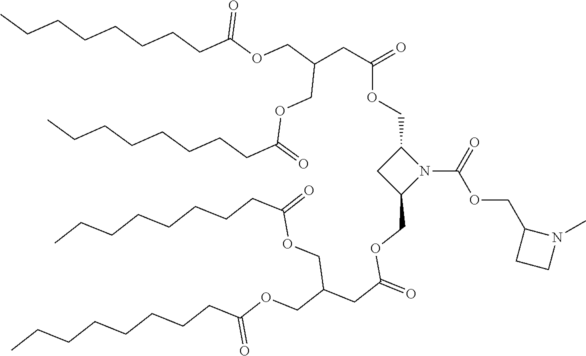 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OCC1CCN1C]

CICL-222-92

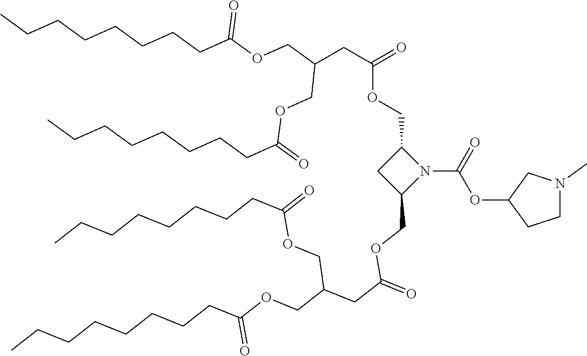 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OC1CCN(C)C1]

CICL-222-93

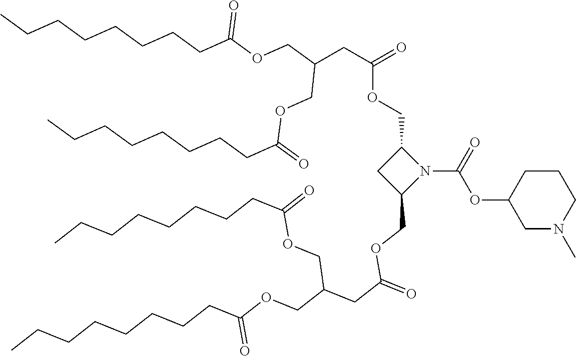 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OC1CCCN(C)C1]

CICL-222-95

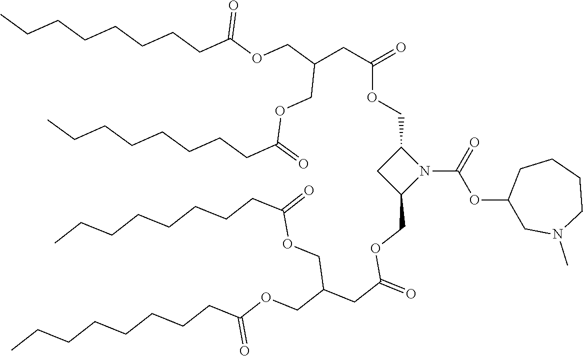 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OC1CCCCN(C)C1]

CICL-222-97

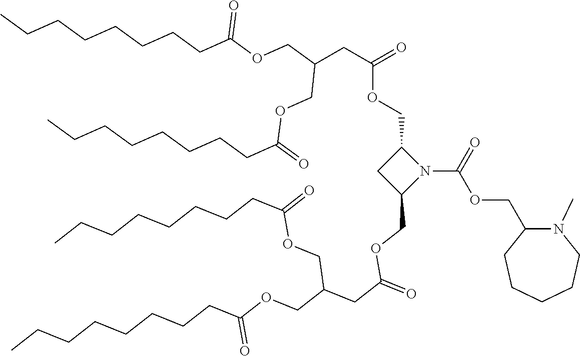 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OCC1CCCCCN1C]

CICL-222-98

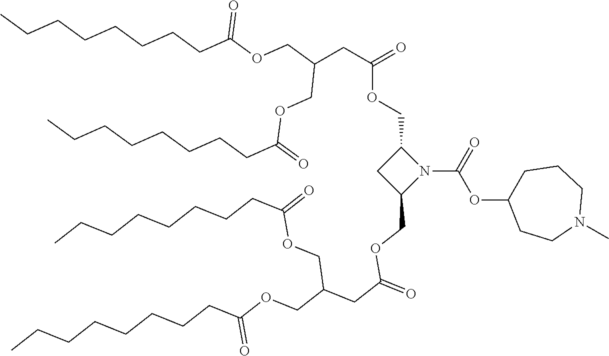 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OC1CCCN(C)CC1]

CICL-222-105

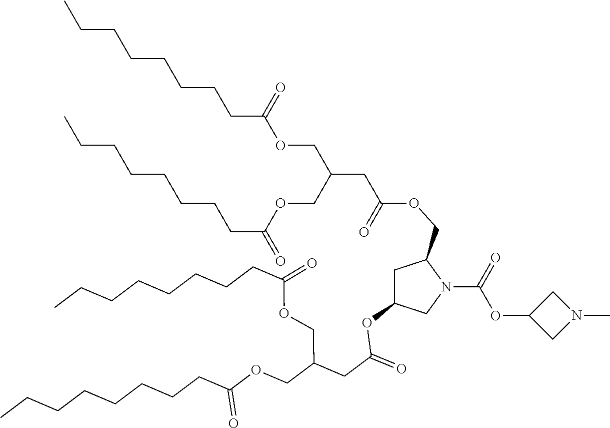 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OC1CN(C)C1]

CICL-224-91

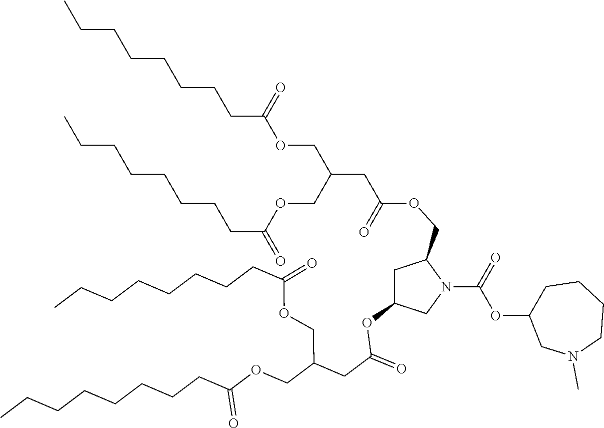 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OC1CCCCN(C)C1]

CICL-224-97

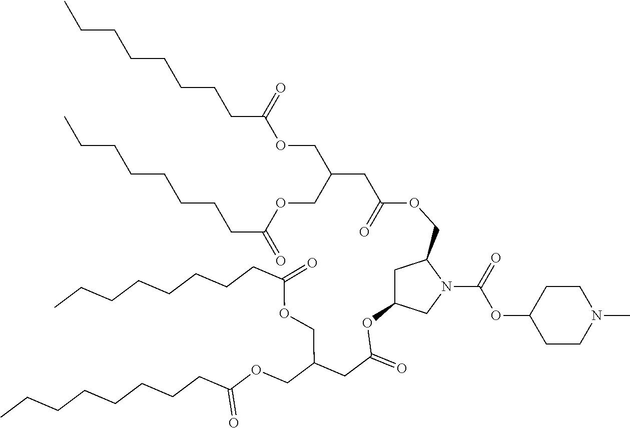 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OC1CCN(C)CC1]

CICL-224-102

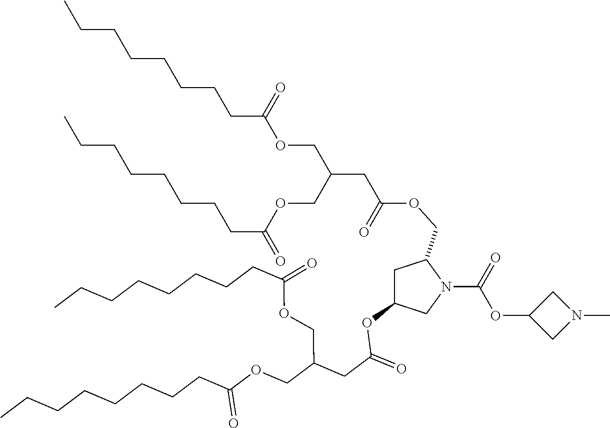 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OC1CN(C)C1]

CICL-225-91

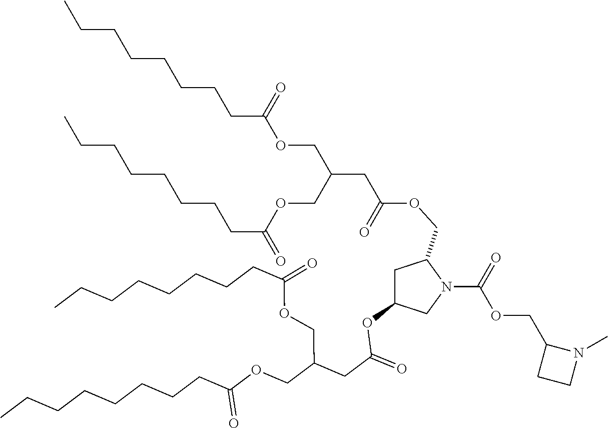 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCC1CCN1C]

CICL-225-92

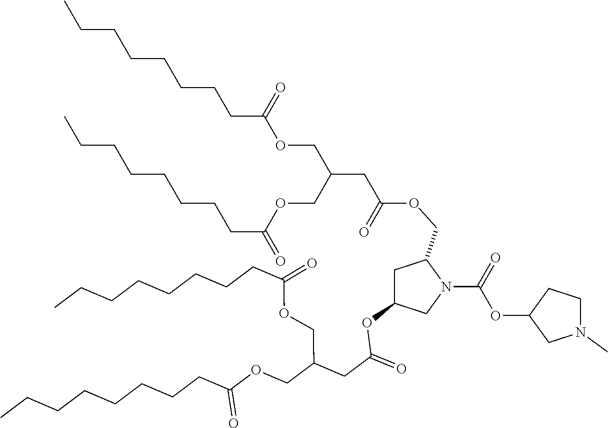 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OC1CCN(C)C1]

CICL-225-93

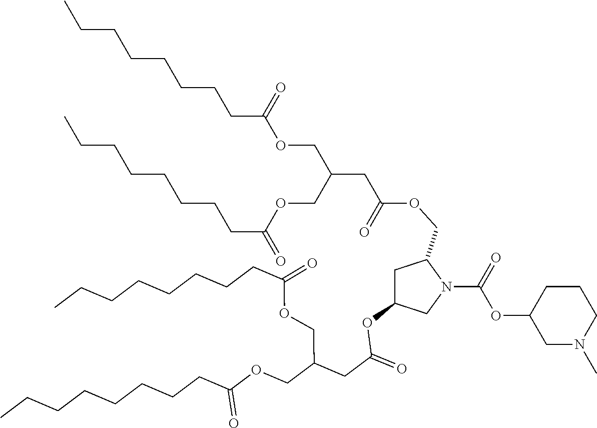 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OC1CCCN(C)C1]

CICL-225-95

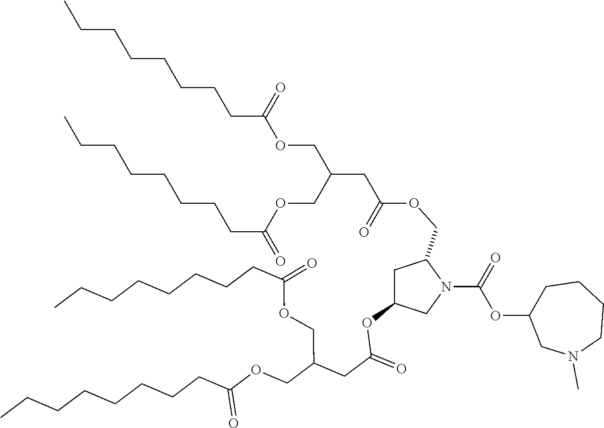 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OC1CCCCN(C)C1]

CICL-225-97

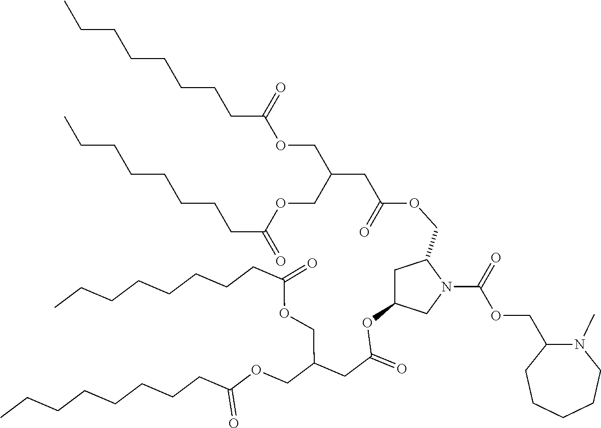 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCC1CCCCCN1C]

CICL-225-98

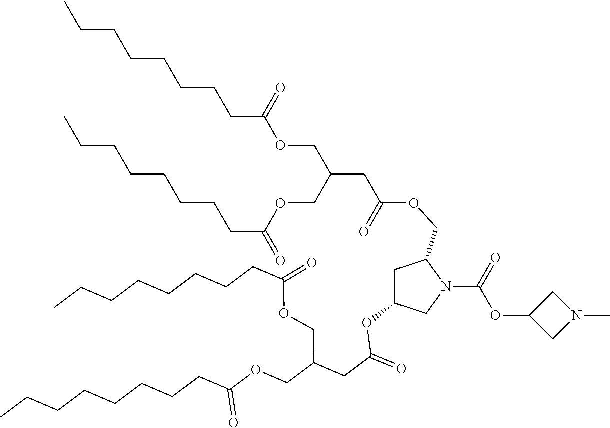 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OC1CN(C)C1]

CICL-223-91

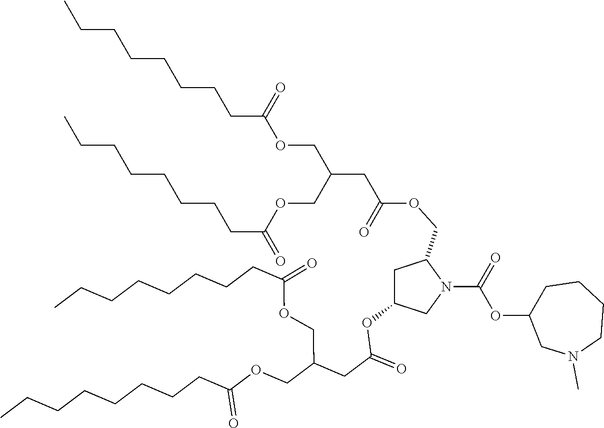 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OC1CCCCN(C)C1]

CICL-223-97

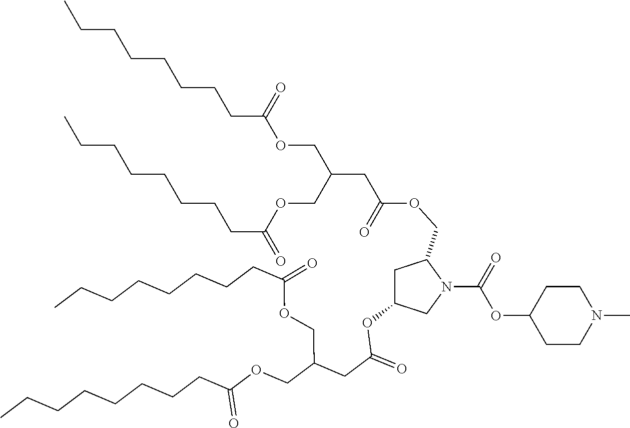 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OC1CCN(C)CC1]

CICL-223-102

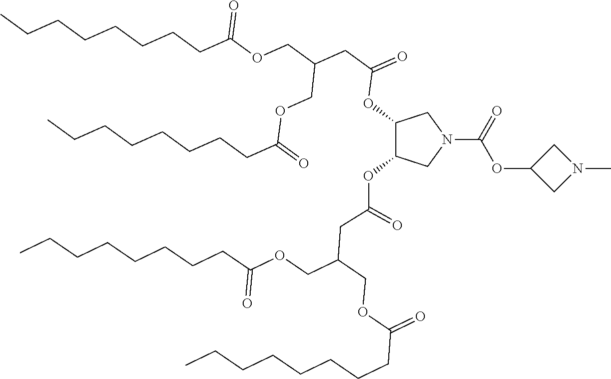 [SMILES: CCCCCCCCC(=O)OCC(COC(=O)CCCCCCCC)CC(=O)O[C@H]1CN(C(=O)OC2CN(C)C2)C[C@H]1OC(=O)CC(COC(=O)CCCCCCCC)COC(=O)CCCCCCCC]

CICL-216-91

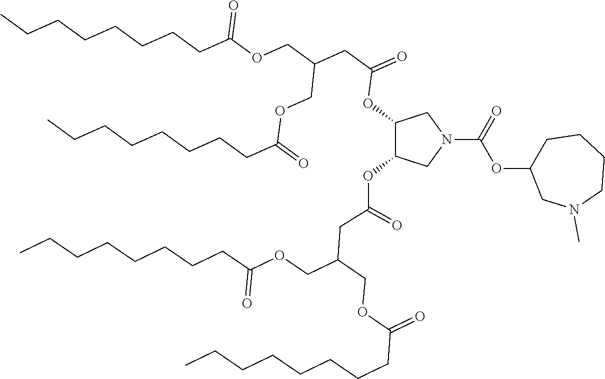 [SMILES: CCCCCCCCC(=O)OCC(COC(=O)CCCCCCCC)CC(=O)O[C@H]1CN(C(=O)OC2CCCCN(C)C2)C[C@H]1OC(=O)CC(COC(=O)CCCCCCCC)COC(=O)CCCCCCCC]

CICL-216-97

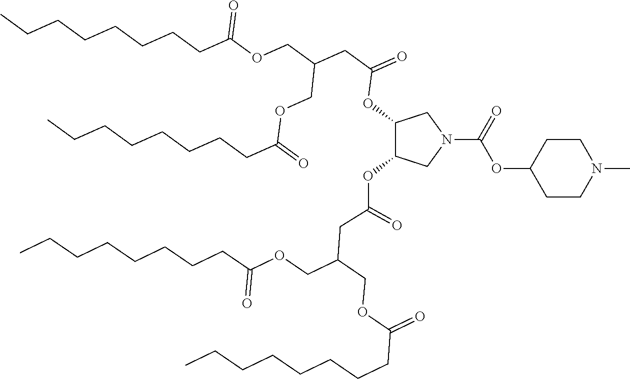 [SMILES: CCCCCCCCC(=O)OCC(COC(=O)CCCCCCCC)CC(=O)O[C@H]1CN(C(=O)OC2CCN(C)CC2)C[C@H]1OC(=O)CC(COC(=O)CCCCCCCC)COC(=O)CCCCCCCC]

CICL-216-102

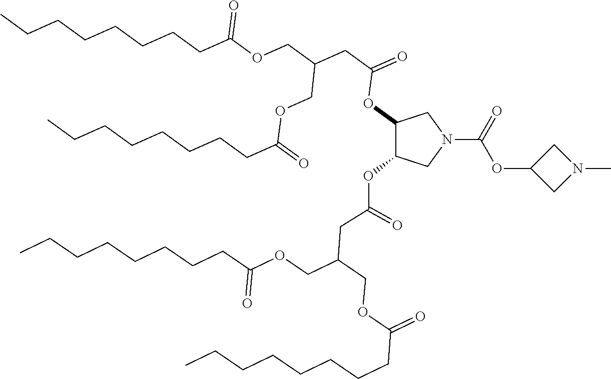 [SMILES: CCCCCCCCC(=O)OCC(COC(=O)CCCCCCCC)CC(=O)O[C@H]1CN(C(=O)OC2CN(C)C2)C[C@@H]1OC(=O)CC(COC(=O)CCCCCCCC)COC(=O)CCCCCCCC]

CICL-215-91

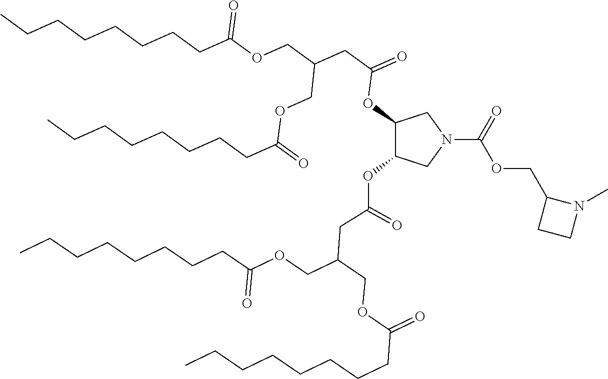 [SMILES: CCCCCCCCC(=O)OCC(COC(=O)CCCCCCCC)CC(=O)O[C@H]1CN(C(=O)OCC2CCN2C)C[C@@H]1OC(=O)CC(COC(=O)CCCCCCCC)COC(=O)CCCCCCCC]

CICL-215-92

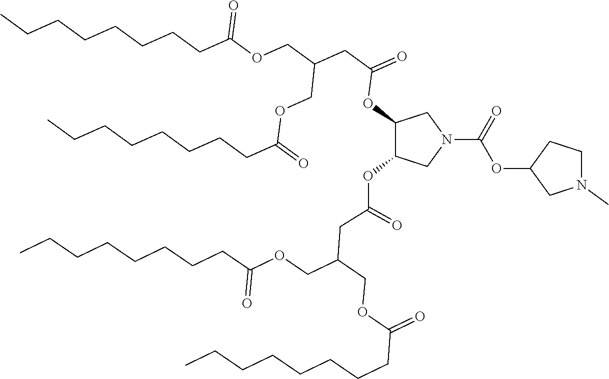 [SMILES: CCCCCCCCC(=O)OCC(COC(=O)CCCCCCCC)CC(=O)O[C@H]1CN(C(=O)OC2CCN(C)C2)C[C@@H]1OC(=O)CC(COC(=O)CCCCCCCC)COC(=O)CCCCCCCC]

CICL-215-93

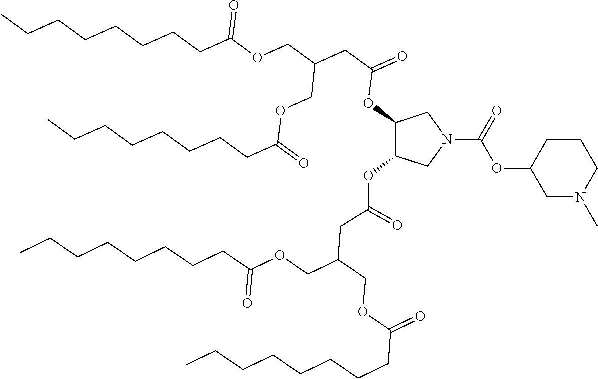 [SMILES: CCCCCCCCC(=O)OCC(COC(=O)CCCCCCCC)CC(=O)O[C@H]1CN(C(=O)OC2CCCN(C)C2)C[C@@H]1OC(=O)CC(COC(=O)CCCCCCCC)COC(=O)CCCCCCCC]

CICL-215-95

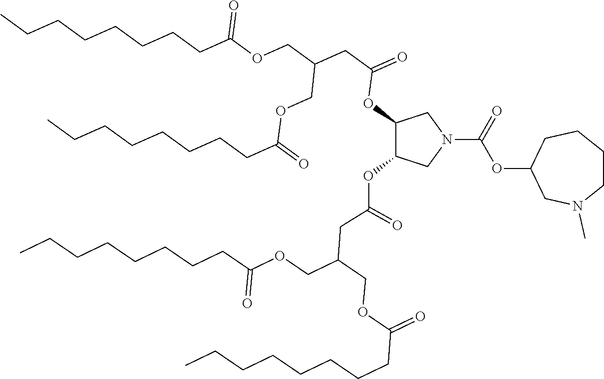 [SMILES: CCCCCCCCC(=O)OCC(COC(=O)CCCCCCCC)CC(=O)O[C@H]1CN(C(=O)OC2CCCCN(C)C2)C[C@@H]1OC(=O)CC(COC(=O)CCCCCCCC)COC(=O)CCCCCCCC]

CICL-215-97

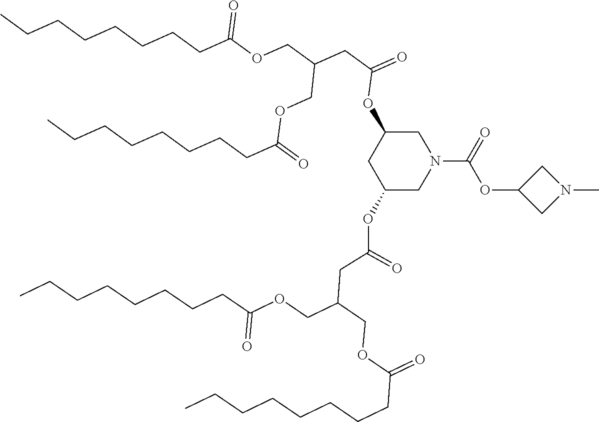 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OC2CN(C)C2)C1]

CICL-220-91

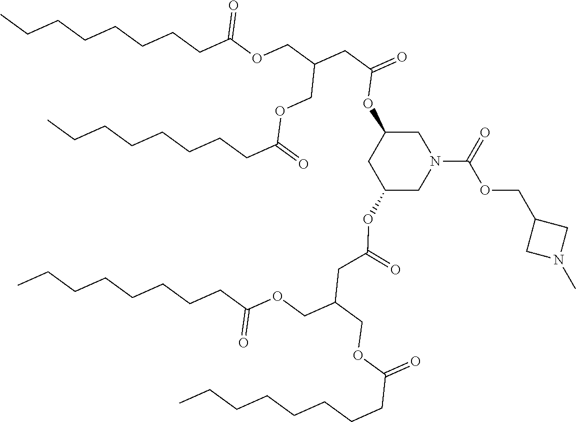 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCC2CN(C)C2)C1]

CICL-220-92

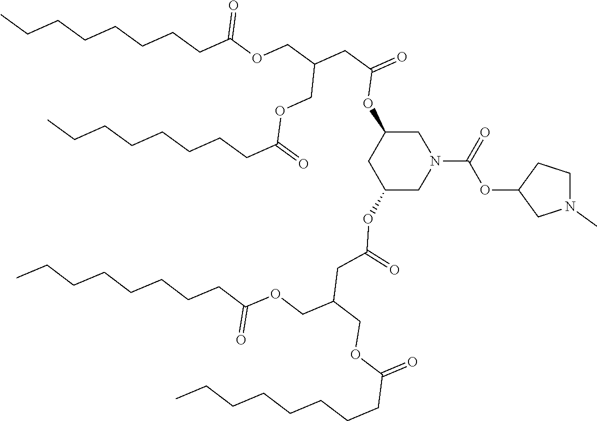 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OC2CCN(C)C2)C1]

CICL-220-93

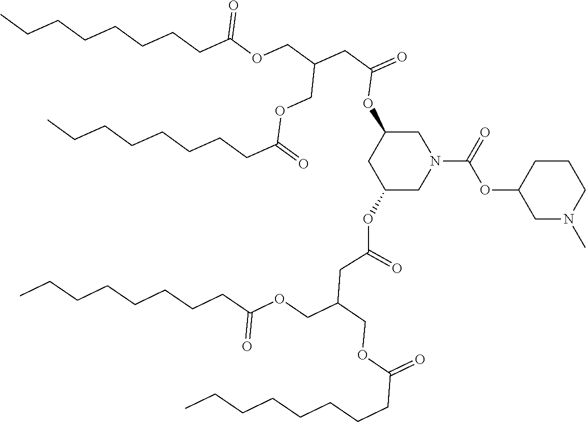 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OC2CCCN(C)C2)C1]

CICL-220-95

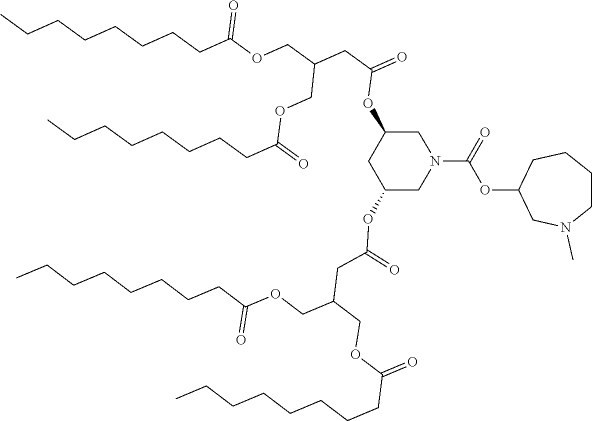 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OC2CCCCN(C)C2)C1]

CICL-220-97

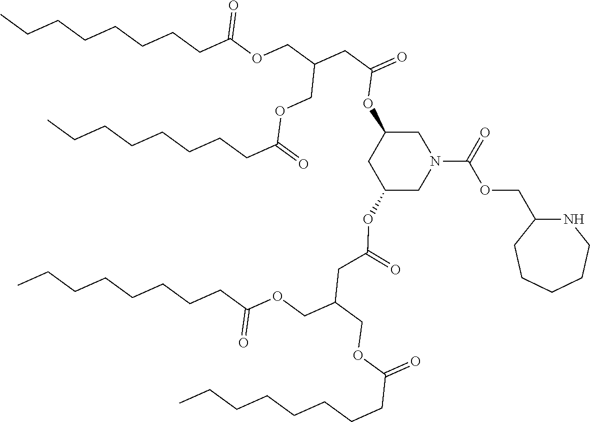 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCC2CCCCCN2)C1]

CICL-220-98

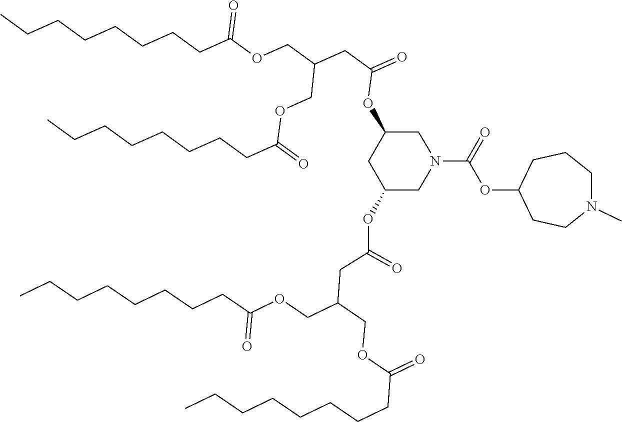 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OC2CCCN(C)CC2)C1]

CICL-220-105

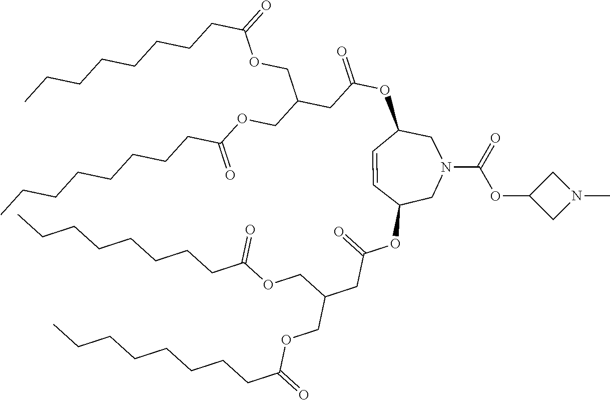

CICL-219-91

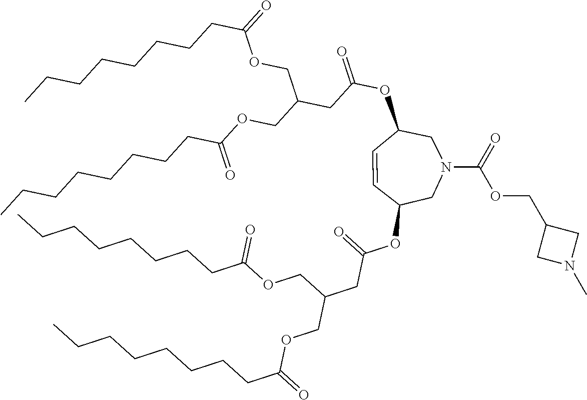

CICL-219-92

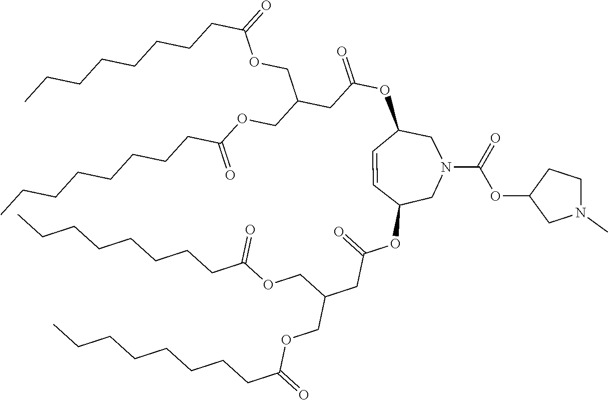

CICL-219-93

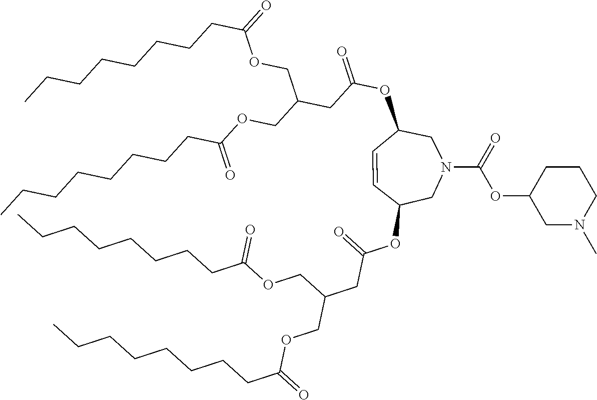

CICL-219-95

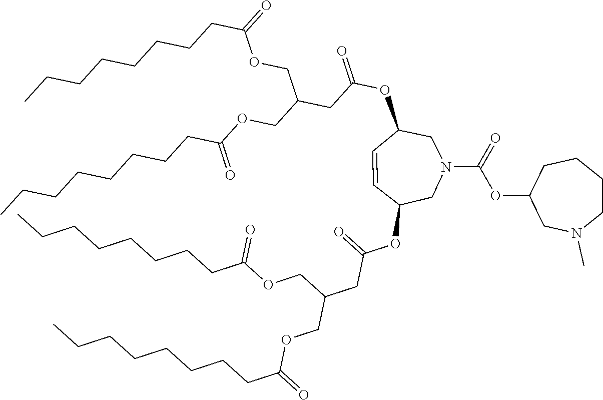

CICL-219-97

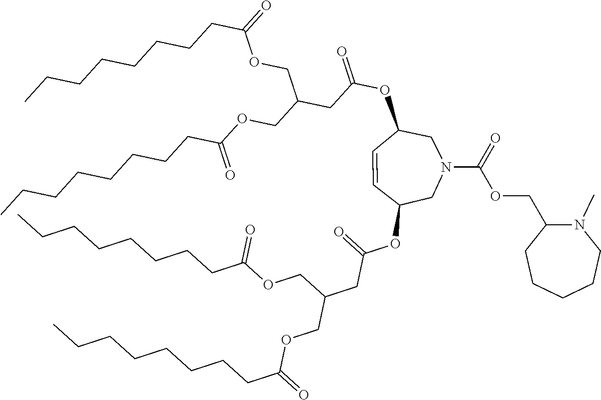

CICL-219-98

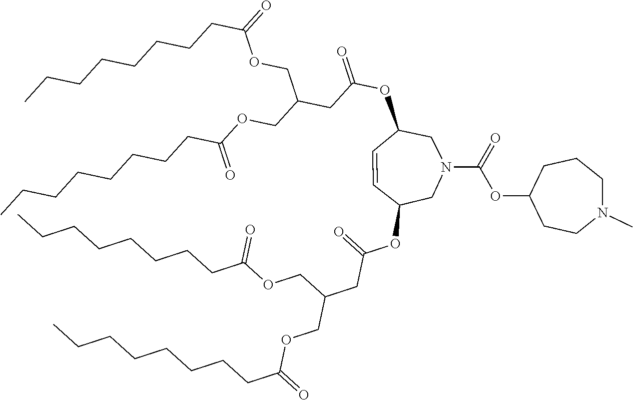

CICL-219-105

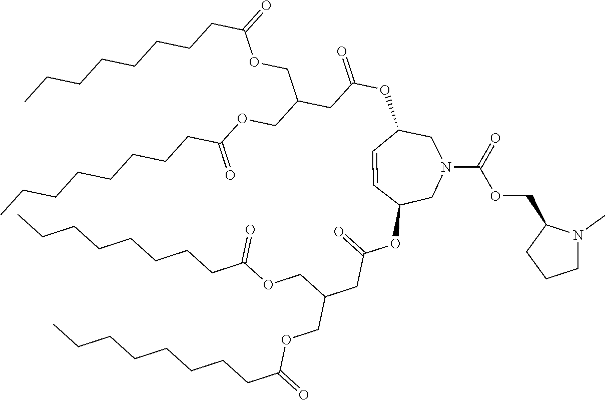

CICL-218-94

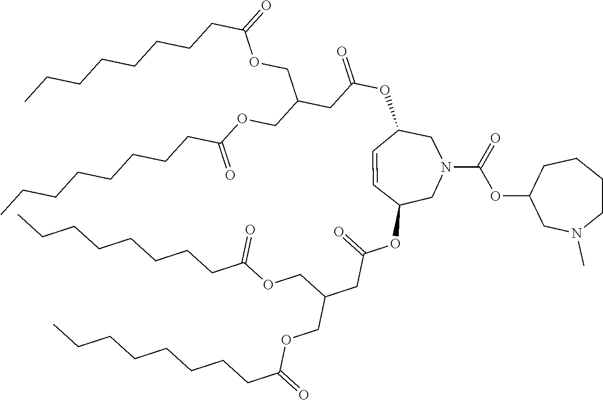

CICL-218-96

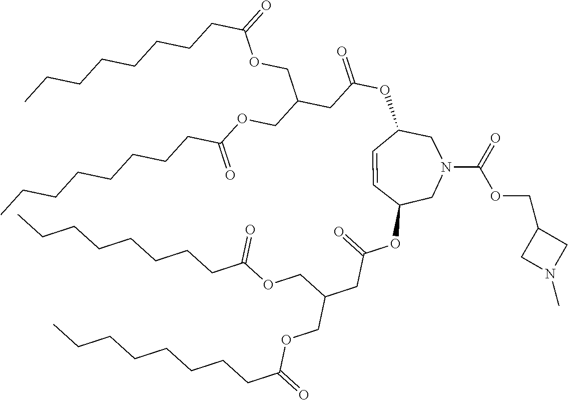

CICL-218-99

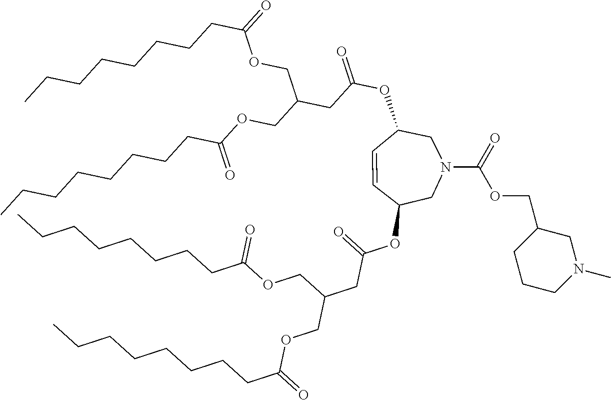

CICL-218-103

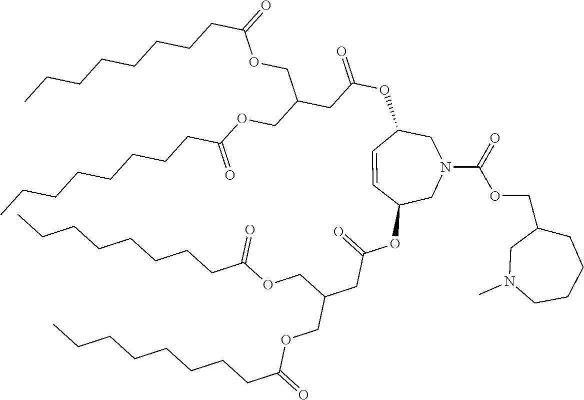

CICL-218-106

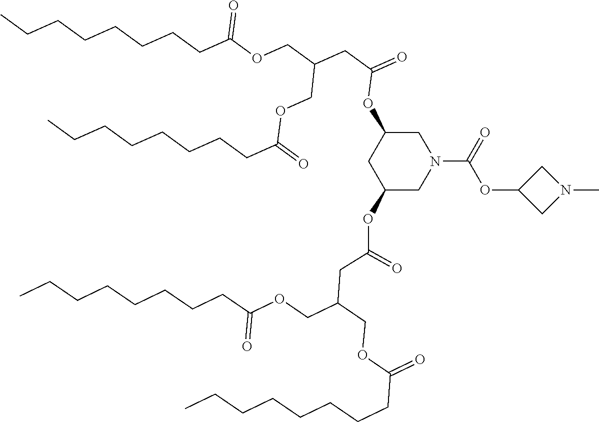 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OC2CN(C)C2)C1]

CICL-238-91

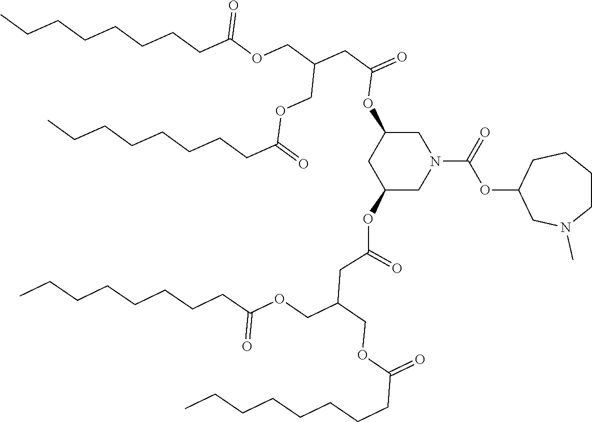 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OC2CCCCN(C)C2)C1]

CICL-238-97

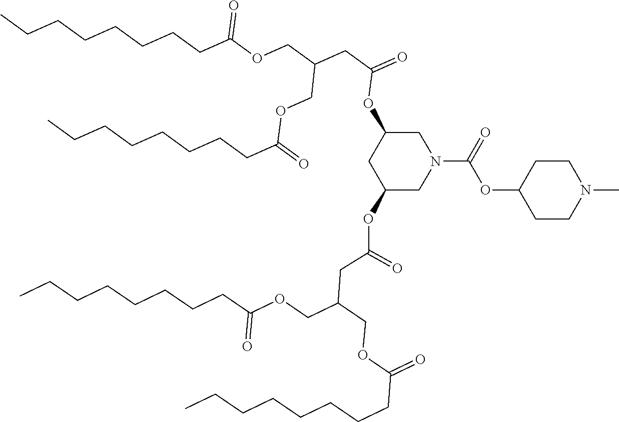 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OC2CCN(C)CC2)C1]

CICL-238-102

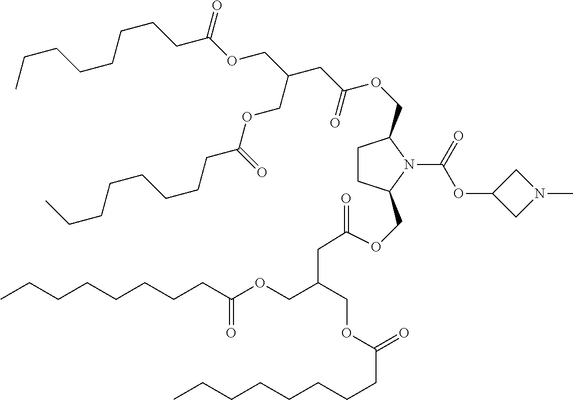 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OC1CN(C)C1]

CICL-242-91

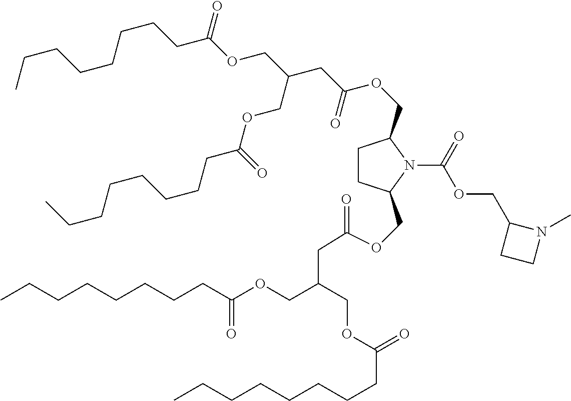 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCC1CCN1C]

CICL-242-92

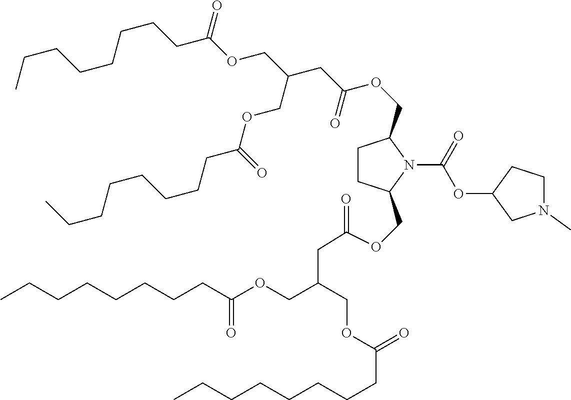 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OC1CCN(C)C1]

CICL-242-93

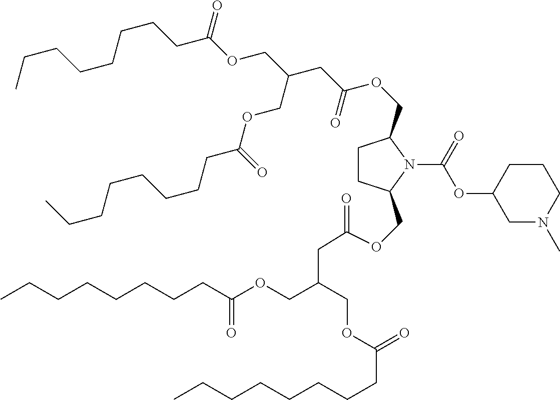 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OC1CCCN(C)C1]

CICL-242-95

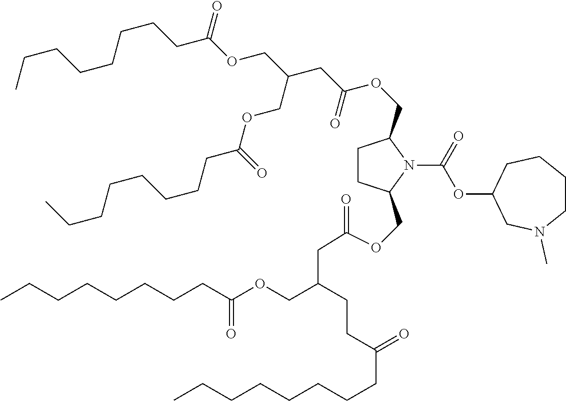 [SMILES: CCCCCCCCC(=O)CCC(COC(=O)CCCCCCCC)CC(=O)OC[C@H]1CC[C@@H](COC(=O)CC(COC(=O)CCCCCCCC)COC(=O)CCCCCCCC)N1C(=O)OC1CCCCN(C)C1]

CICL-242-97

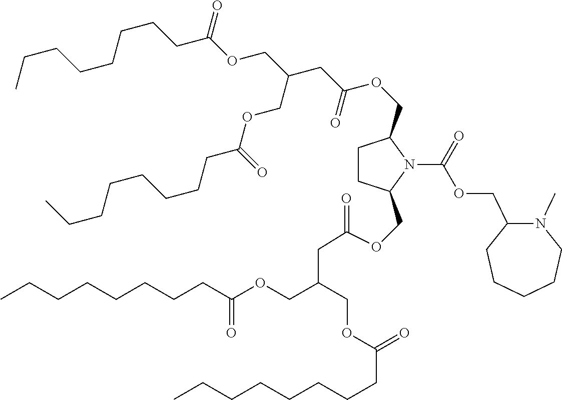 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCC1CCCCCN1C]

CICL-242-98

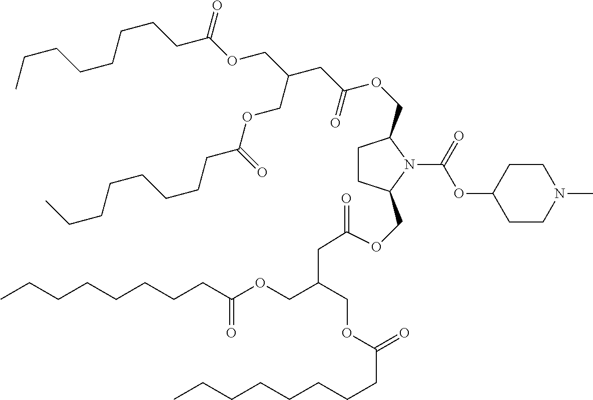 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OC1CCN(C)CC1]

CICL-242-102

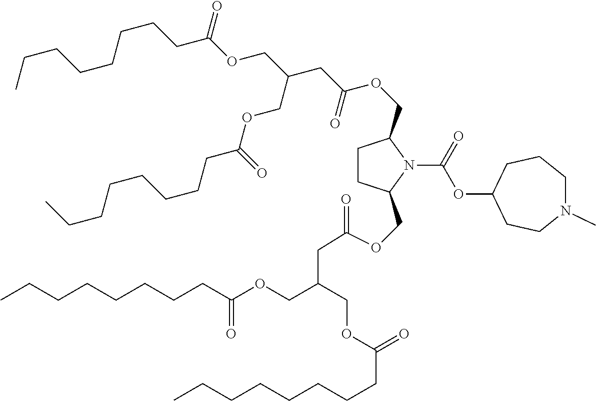 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OC1CCCN(C)CC1]

CICL-242-105

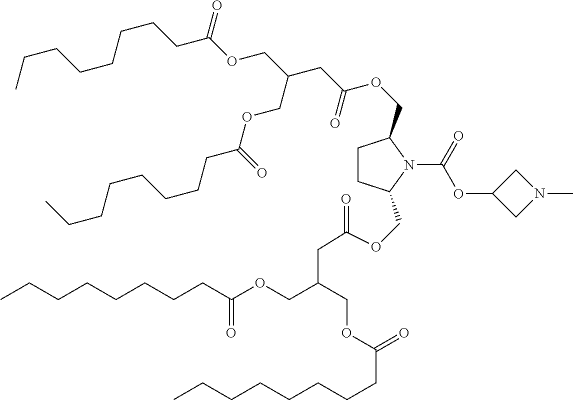 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OC1CN(C)C1]

CICL-243-91

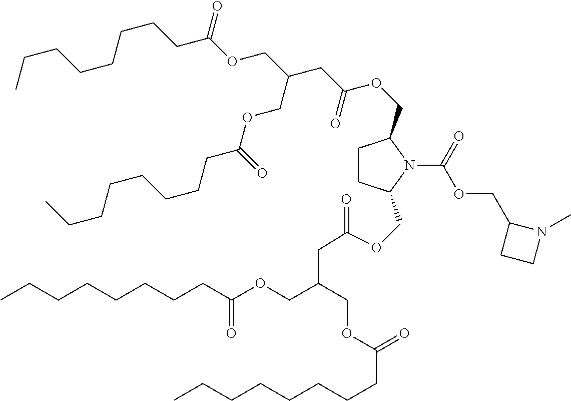 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCC1CCN1C]

CICL-243-92

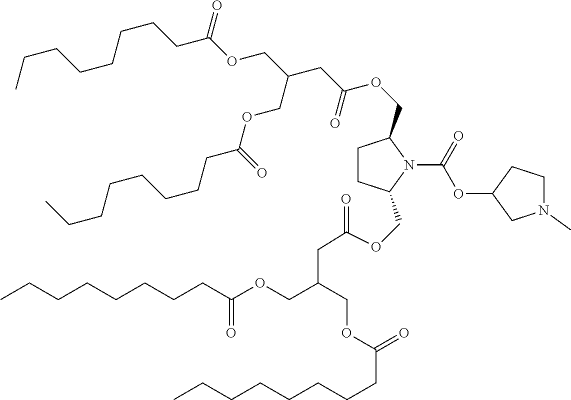 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OC1CCN(C)C1]

CICL-243-93

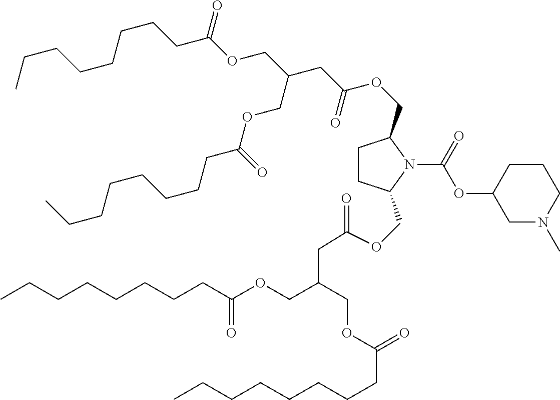 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OC1CCCN(C)C1]

CICL-243-95

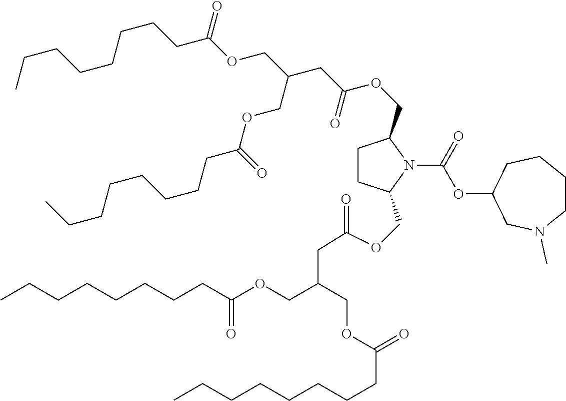 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OC1CCCCN(C)C1]

CICL-243-97

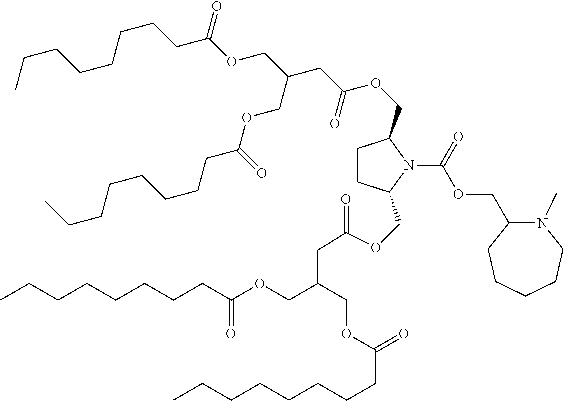 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCC1CCCCCN1C]

CICL-243-98

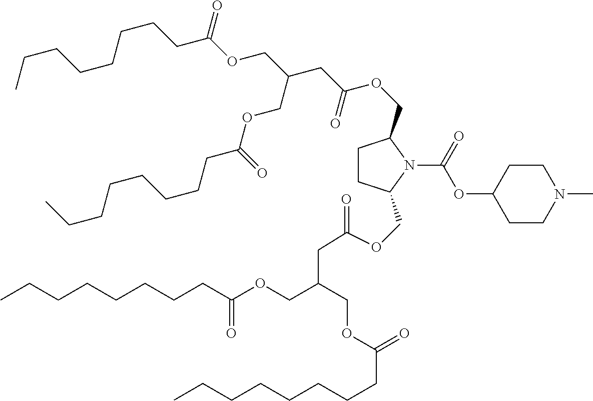 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OC1CCN(C)CC1]

CICL-243-102

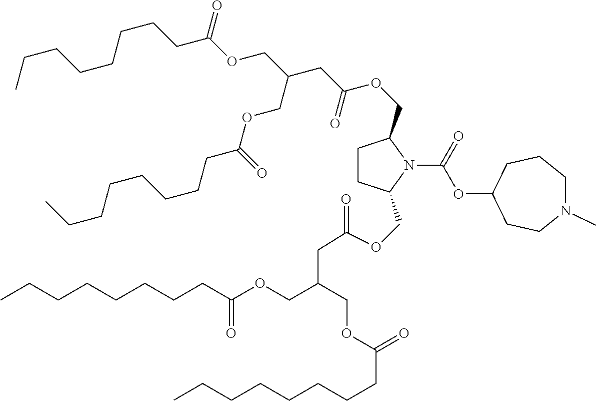 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OC1CCCN(C)CC1]

CICL-243-105

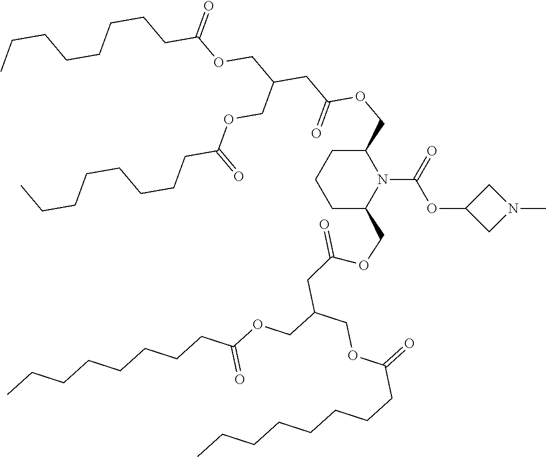 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OC1CN(C)C1]

CICL-244-91

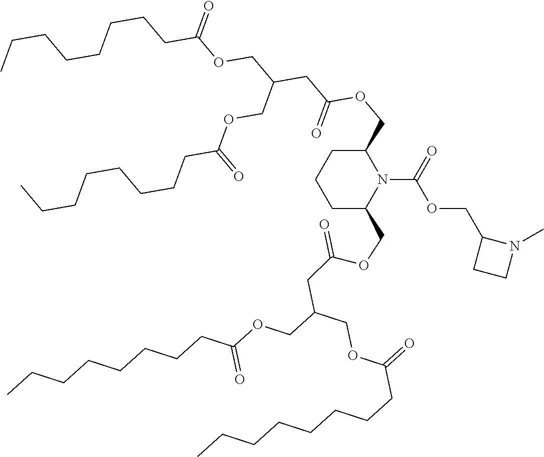 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCC1CCN1C]

CICL-244-92

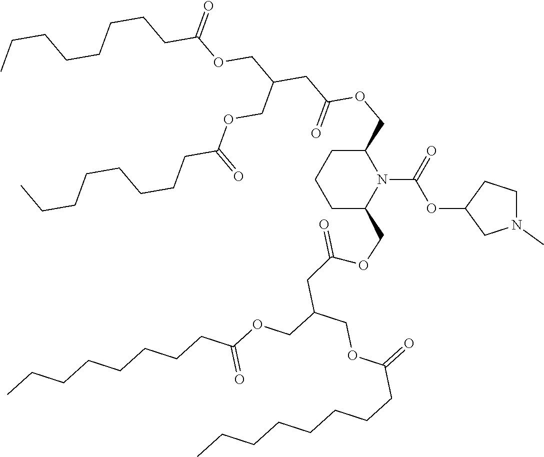 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OC1CCN(C)C1]

CICL-244-93

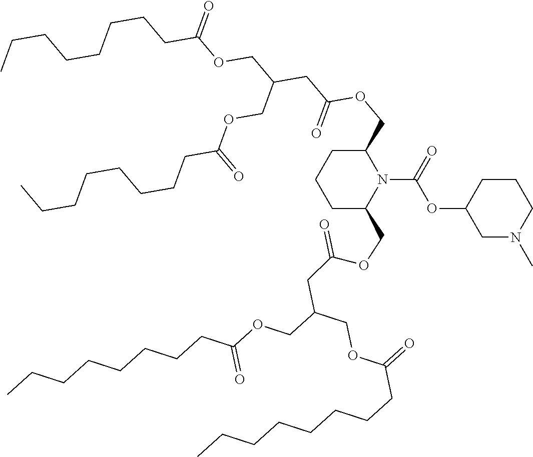 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OC1CCCN(C)C1]

CICL-244-95

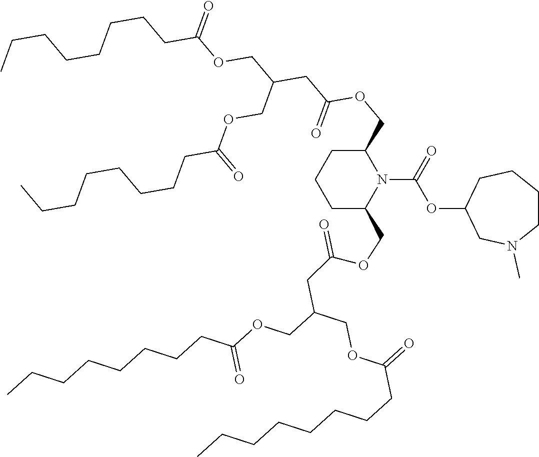 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OC1CCCCN(C)C1]

CICL-244-97

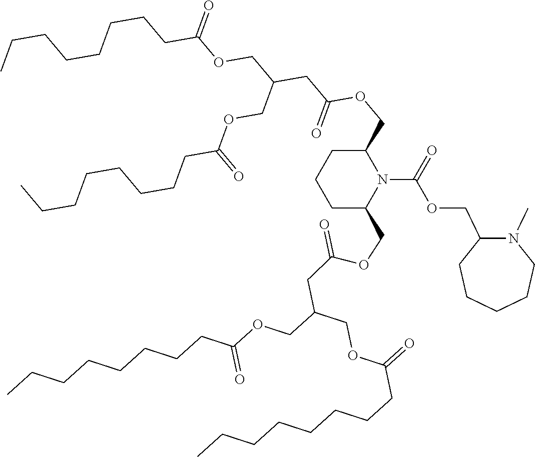 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCC1CCCCCN1C]

CICL-244-98

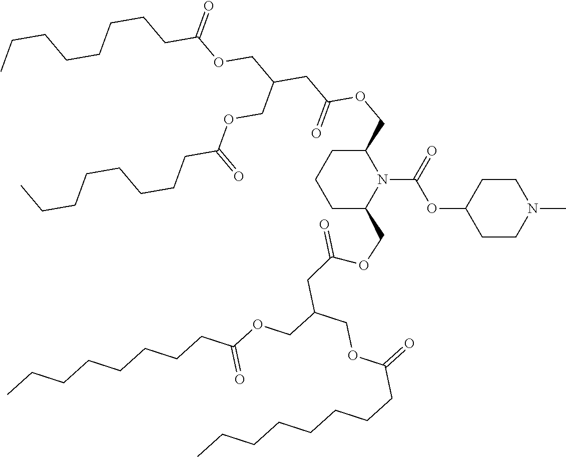 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OC1CCN(C)CC1]

CICL-244-102

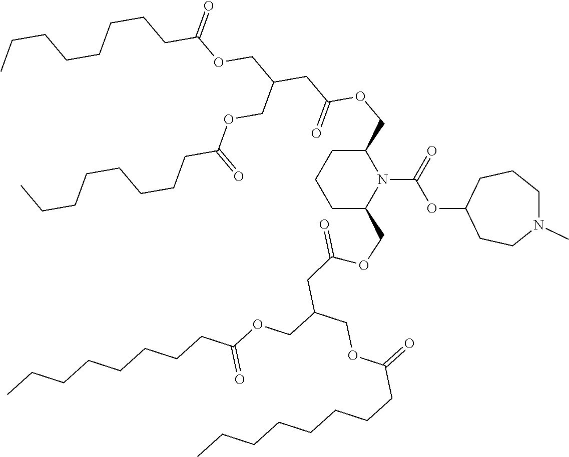 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OC1CCCN(C)CC1]

CICL-244-105

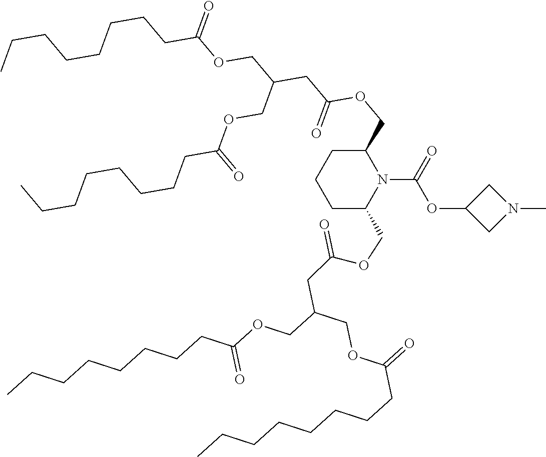 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OC1CN(C)C1]

CICL-245-91

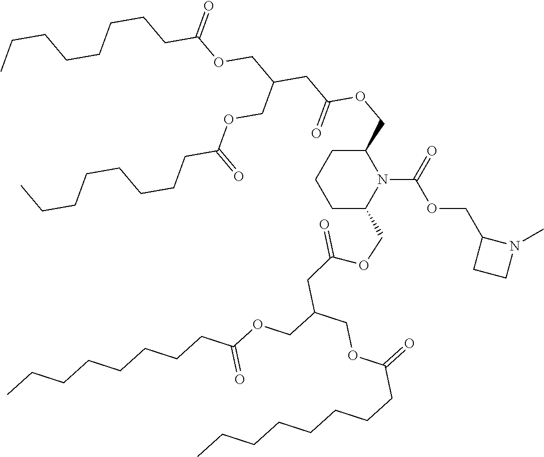 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCC1CCN1C]

CICL-245-92

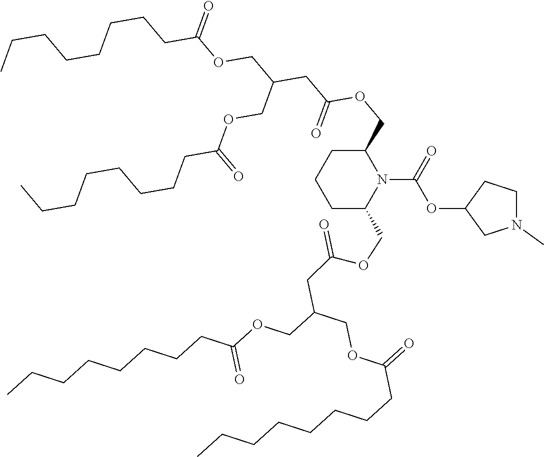 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OC1CCN(C)C1]

CICL-245-93

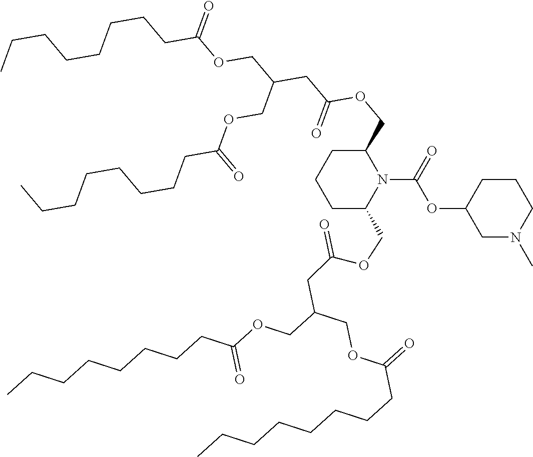 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OC1CCCN(C)C1]

CICL-245-95

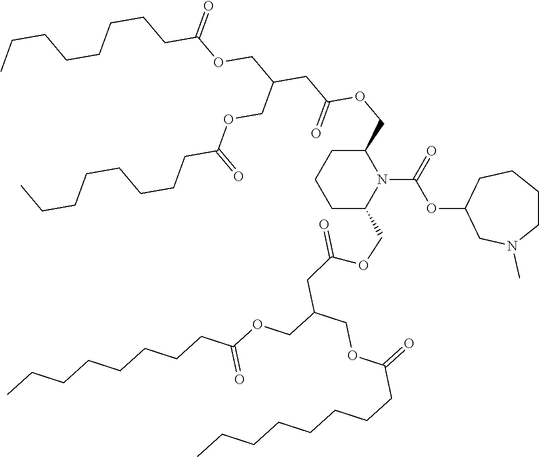 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OC1CCCCN(C)C1]

CICL-245-97

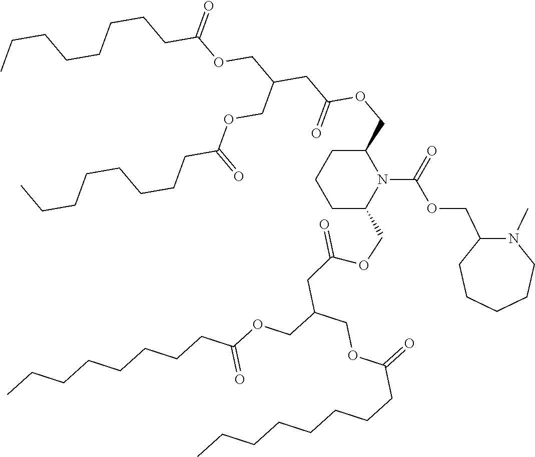 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCC1CCCCCN1C]

CICL-245-98

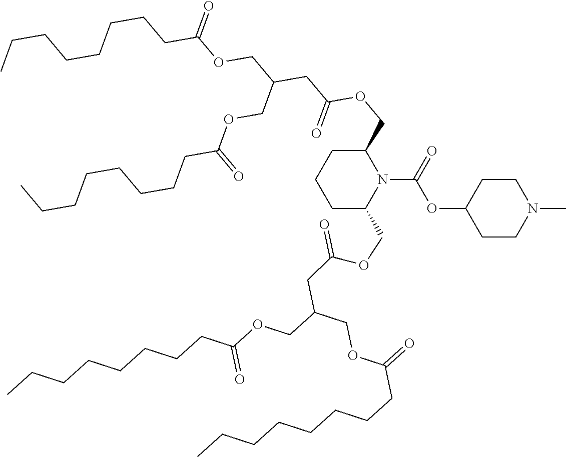 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OC1CCN(C)CC1]

CICL-245-102

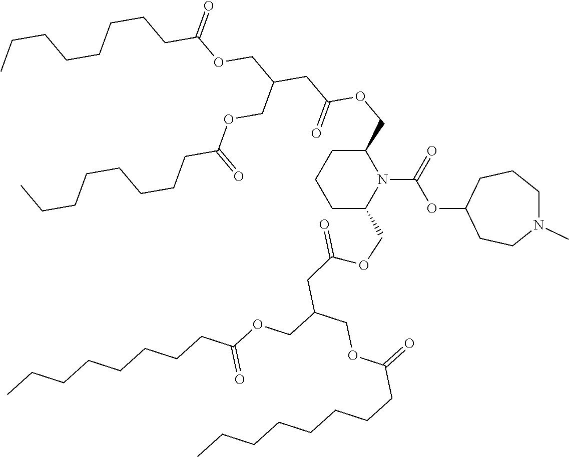 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OC1CCCN(C)CC1]

CICL-245-105

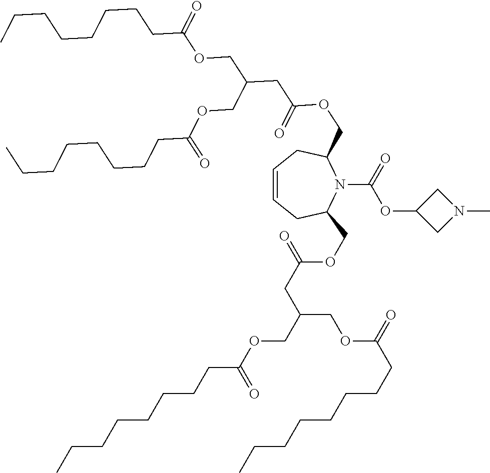 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OC1CN(C)C1]

CICL-246-91

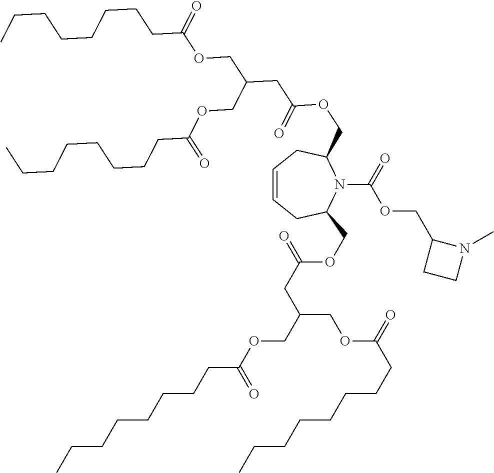 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCC1CCN1C]

CICL-246-92

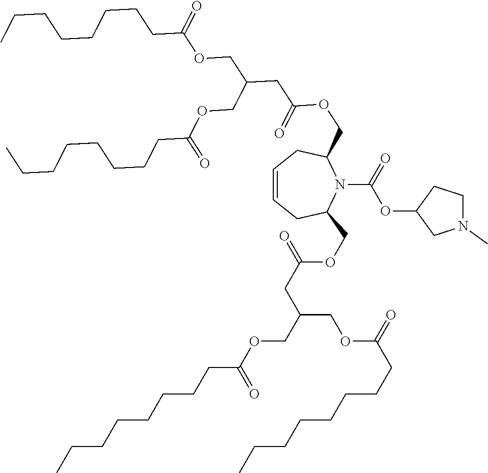 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OC1CCN(C)C1]

CICL-246-93

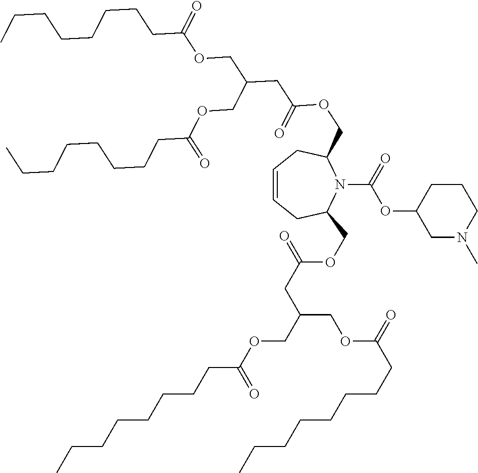 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OC1CCCN(C)C1]

CICL-246-95

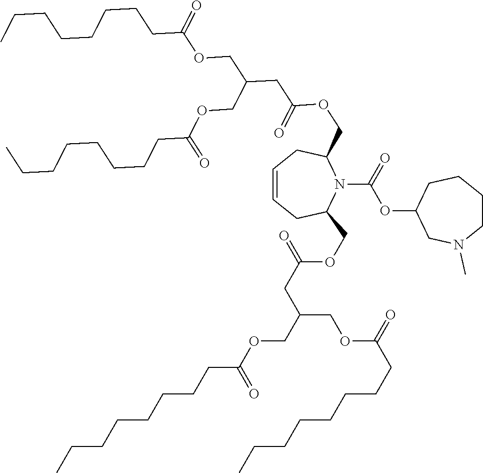 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OC1CCCCN(C)C1]

CICL-246-97

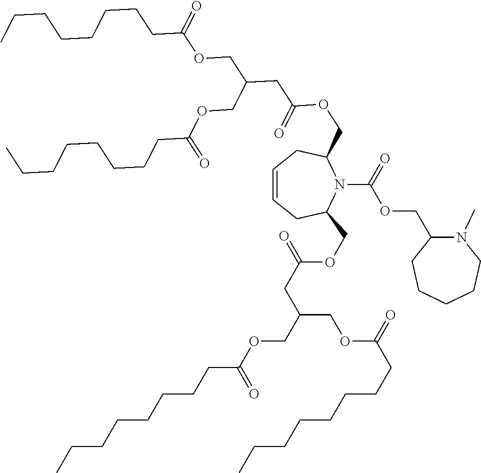 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCC1CCCCCN1C]

CICL-246-98

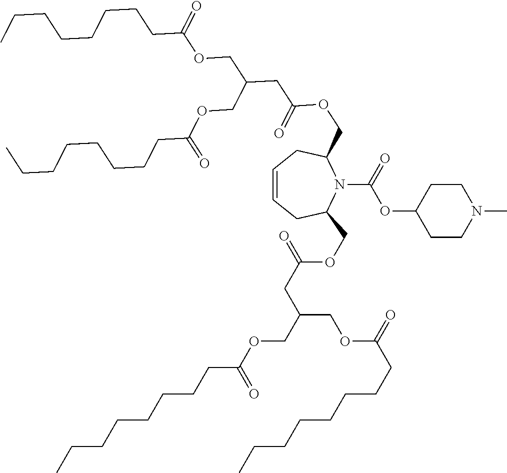 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OC1CCN(C)CC1]

CICL-246-102

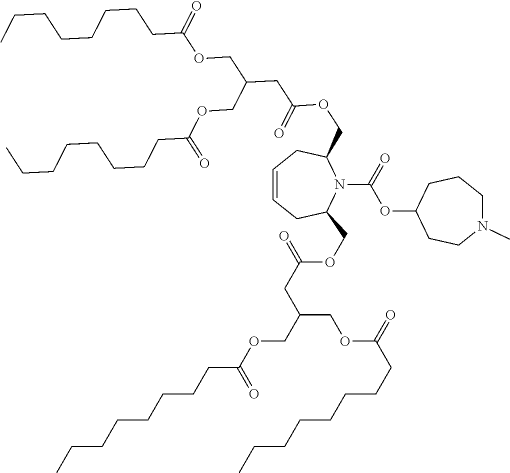 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OC1CCCN(C)CC1]

CICL-246-105

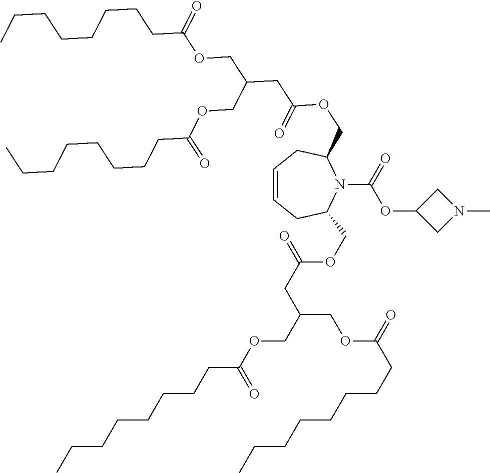 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OC1CN(C)C1]

CICL-247-91

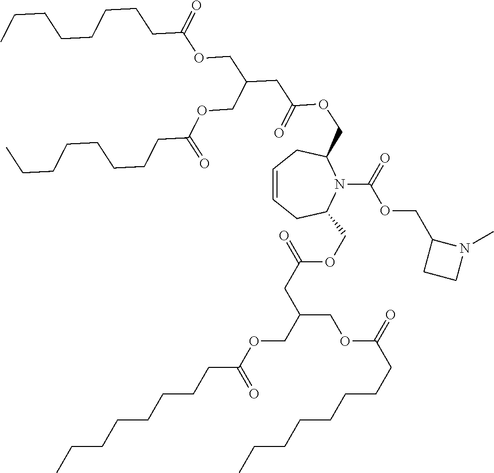 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCC1CCN1C]

CICL-247-92

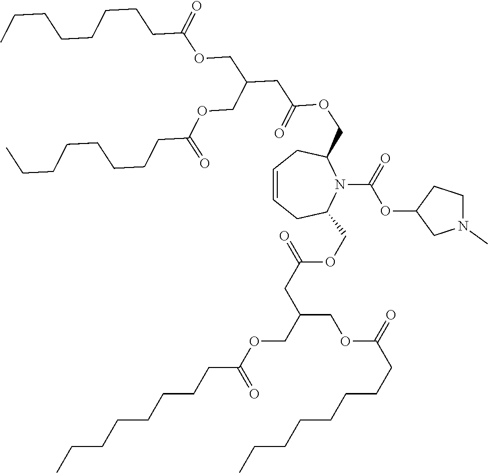 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OC1CCN(C)C1]

CICL-247-93

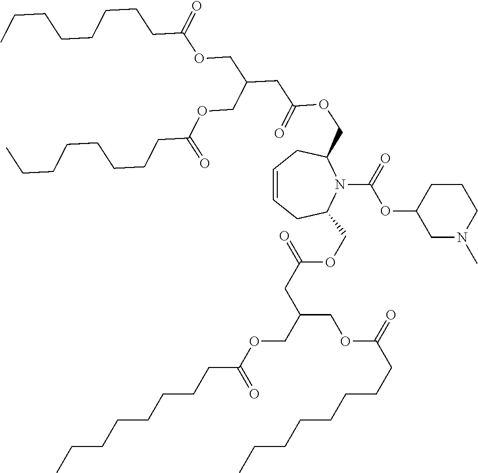 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OC1CCCN(C)C1]

CICL-247-95

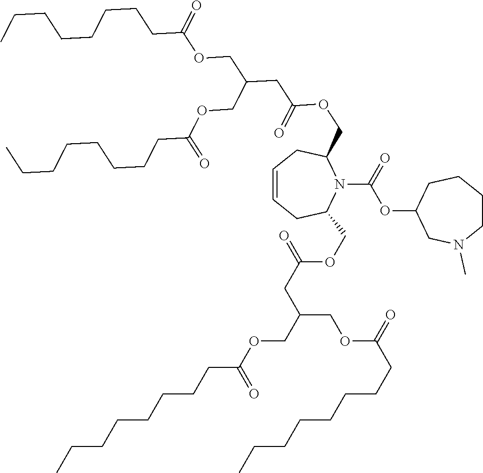 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OC1CCCCN(C)C1]

CICL-247-97

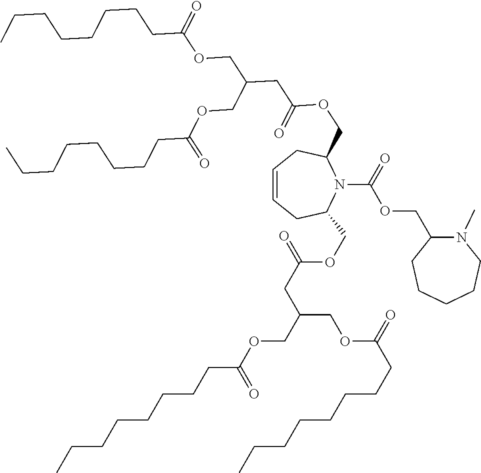 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCC1CCCCCN1C]

CICL-247-98

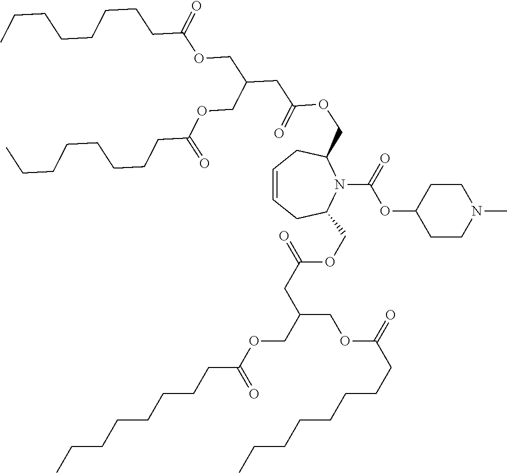 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OC1CCN(C)CC1]

CICL-247-102

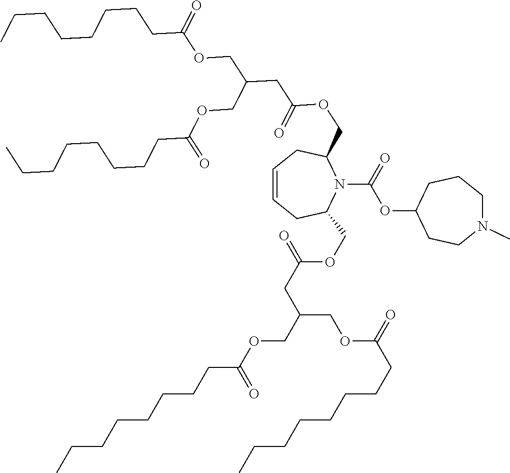 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OC1CCCN(C)CC1]

CICL-247-105

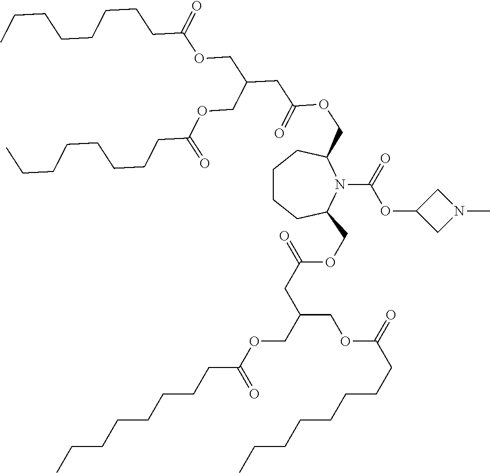 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OC1CN(C)C1]

CICL-248-91

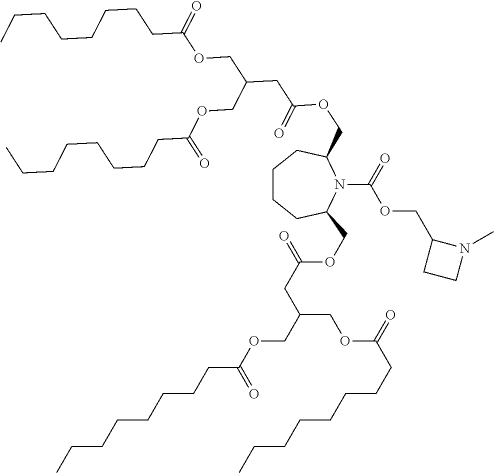 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCC1CCN1C]

CICL-248-92

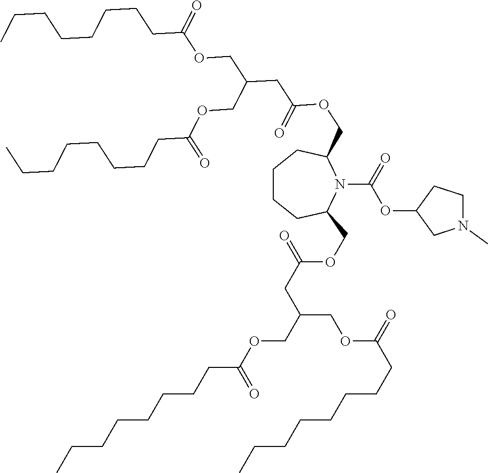 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OC1CCN(C)C1]

CICL-248-93

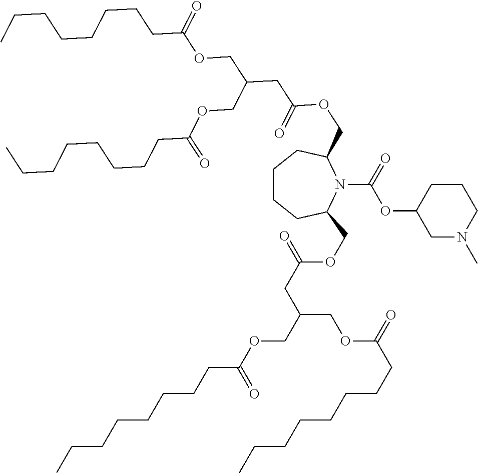 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OC1CCCN(C)C1]

CICL-248-95

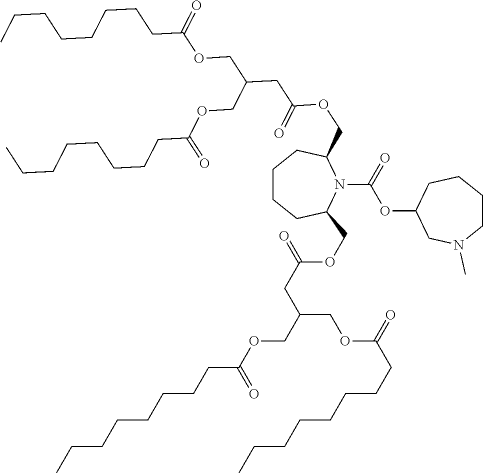 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OC1CCCCN(C)C1]

CICL-248-97

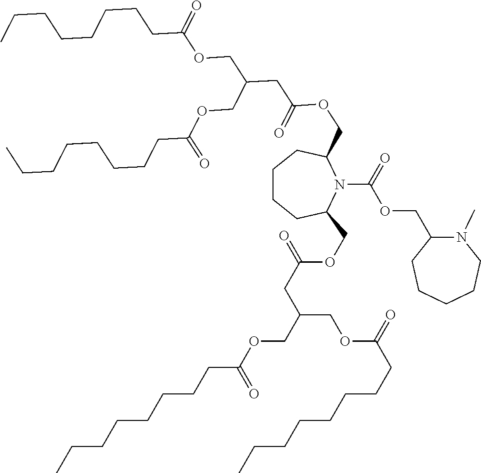 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCC1CCCCCN1C]

CICL-248-98

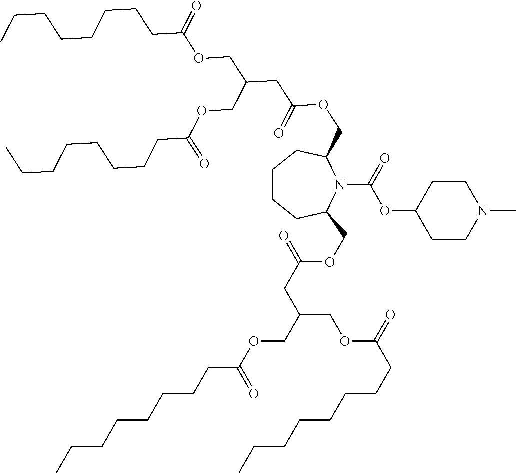 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OC1CCN(C)CC1]

CICL-248-102

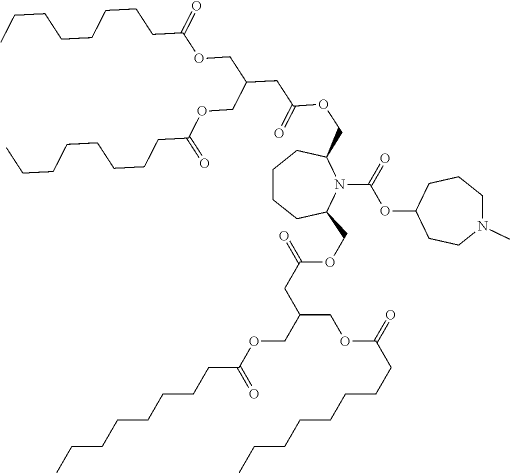 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OC1CCCN(C)CC1]

CICL-248-105

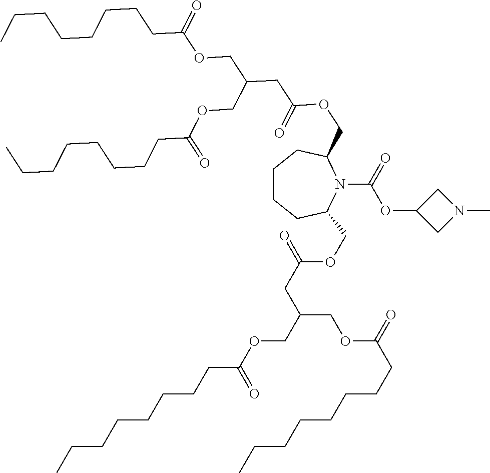 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OC1CN(C)C1]

CICL-249-91

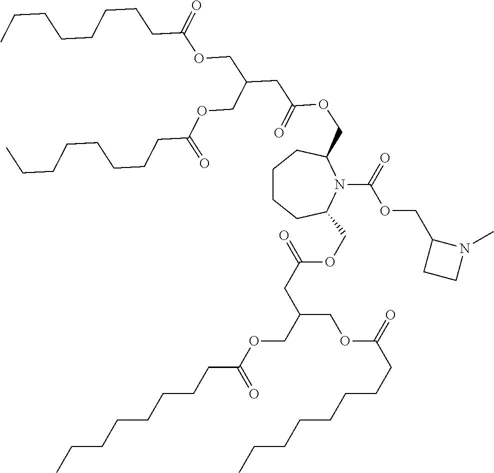 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCC1CCN1C]

CICL-249-92

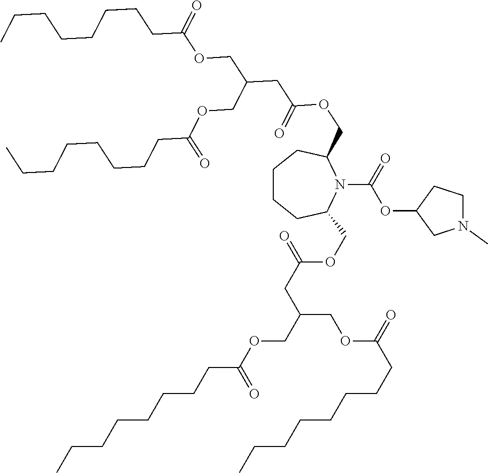 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OC1CCN(C)C1]

CICL-249-93

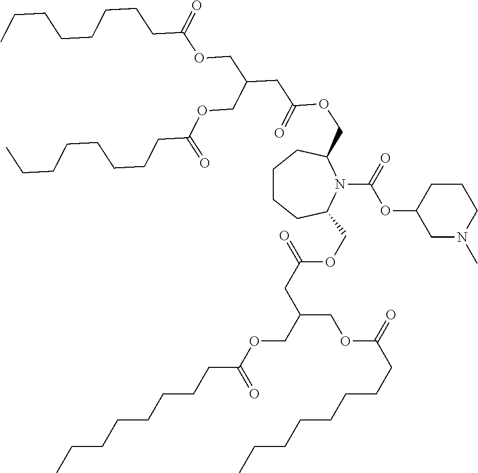 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OC1CCCN(C)C1]

CICL-249-95

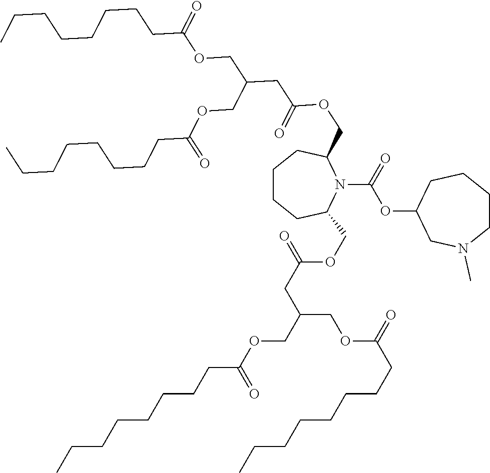 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OC1CCCCN(C)C1]

CICL-249-97

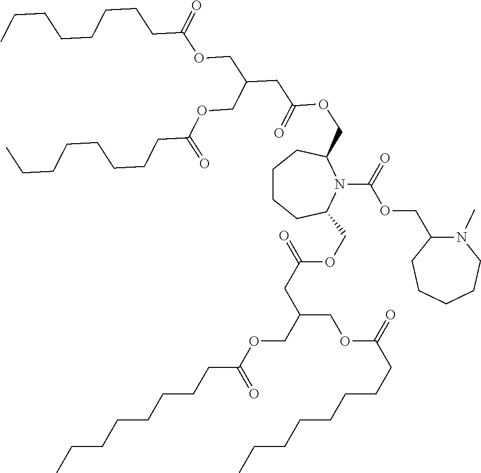 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCC1CCCCCN1C]

CICL-249-98

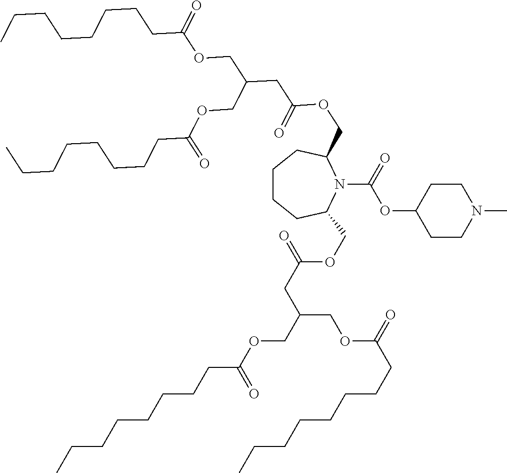 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OC1CCN(C)CC1]

CICL-249-102

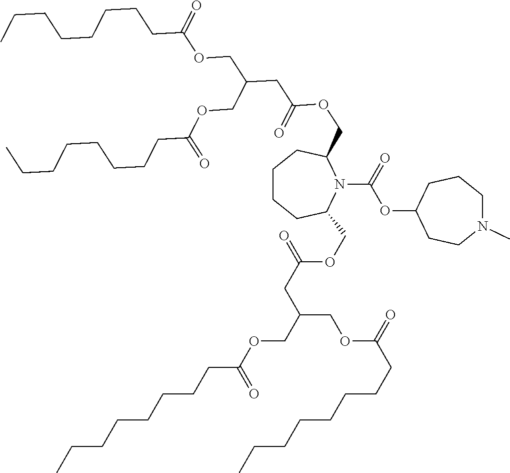 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OC1CCCN(C)CC1]

CICL-249-105

The data presented in Tables 8, 9, and 10 illustrates the impact of the structure of the core acylimidazolide on the measured pKa in formulation of lipids as tLNPs all constructed by the addition of N,N-dimethylamino ethanol to form the basic head group. The measured pKa values range from 6.11 (CICL-218) to 7.09 (CICL-238). As described above, cyclic head groups were appended to the core acylimidazolide 16 (used in the synthesis of CICL-207) and calculated (c-pKa) values were determined. Alcohols were selected to move the measured pKa values for alternate core structures associated with the data of Table 10, toward the midpoint of the target range of 6-7 (Table 4). In the description of Formula M2 (above) the definition of groups X includes further substituents which can be appended to a core structure to form ionizable lipids M2. The examination of the connection of a subset of the groups X, when appended to acylimidazolide 16, instructs the selection of groups X to be appended to the core acylimidazolides 7, 12, 16, 20, 24, 28, 32, 36, 40, 44, 48, 53, 57, 61, 65, 69, 76, 80, 85 and 90 in order to target the midpoint of the measured pKa range of 6-7 for the construction of advantageous ionizable cationic lipids. Table 5 illustrates the products of the combination of acylimidazolide 16 with head groups 108-133 (selected from the definition of X in the definition of Formula M2) to form lipids CICL-207-108 through CICL-207-133, with associated c-pKa data.

TABLE 5
Lipids formed from the Combination of Core Acylimidazolide 7 with head groups
selected from 108 through 133.

| X Precursor | Lipid | c-pKa |
|---|---|---|
| | 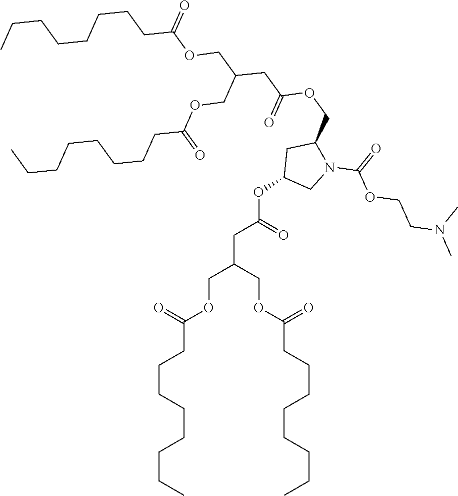 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)C] | 8.42 |
| | CICL-207 | |
| 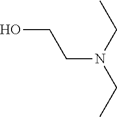 | 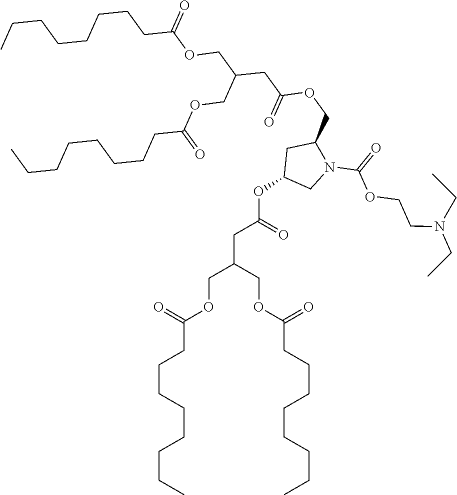 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(CC)CC] | 9.39 |
| 108 | CICL-207-108 | |
| 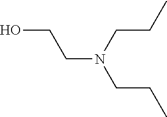 | 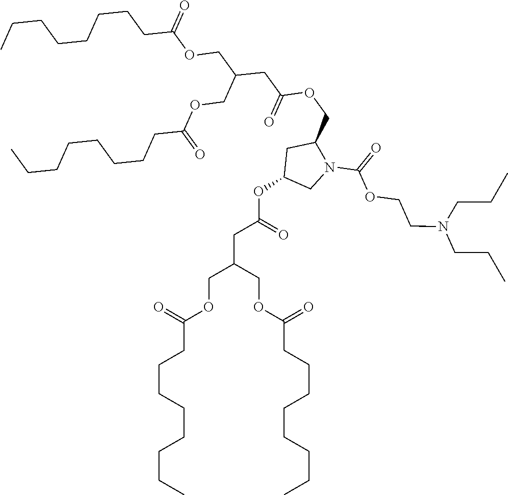 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(CCC)CCC] | 8.58 |
| 109 | CICL-207-109 | |
| 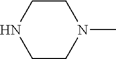 | 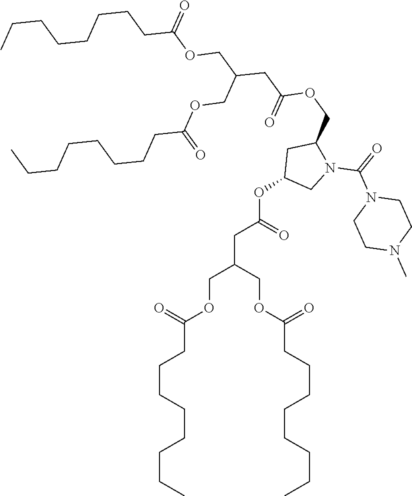 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N1CCN(C)CC1] | 7.16 |
| 110 | CICL-207-110 | |
| 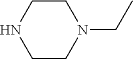 | 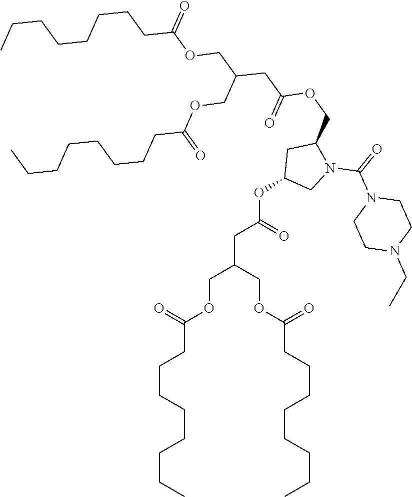 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N1CCN(CC)CC1] | 7.65 |
| 111 | CICL-207-111 | |
| 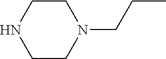 | 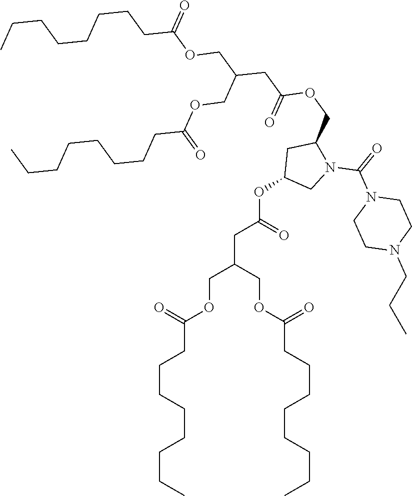 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N1CCN(CCC)CC1] | 7.59 |
| 112 | CICL-207-112 | |
| 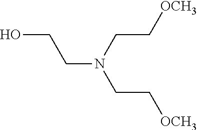 [SMILES: COCCN(CCO)CCOC] | 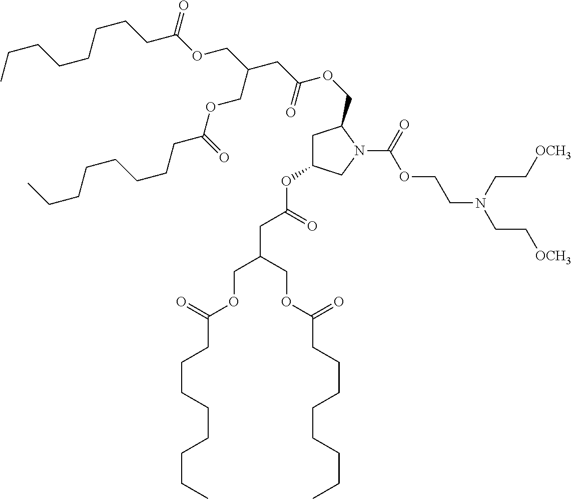 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(CCOC)CCOC] | 6.68 |
| 113 | CICL-207-113 | |
| 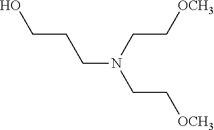 [SMILES: COCCN(CCCO)CCOC] | 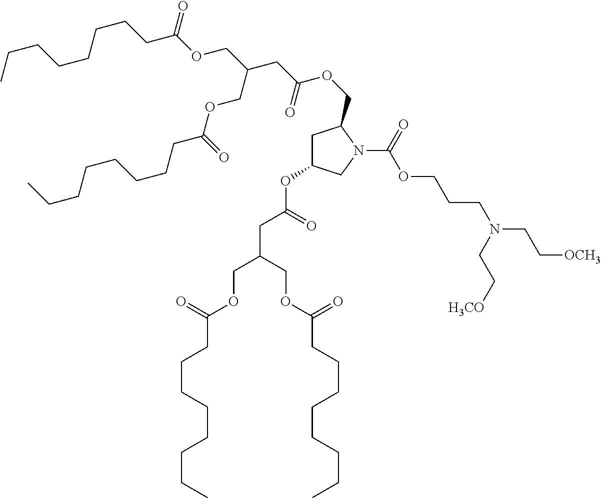 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN(CCOC)CCOC] | 7.50 |
| 114 | CICL-207-114 | |
| 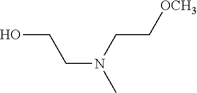 | 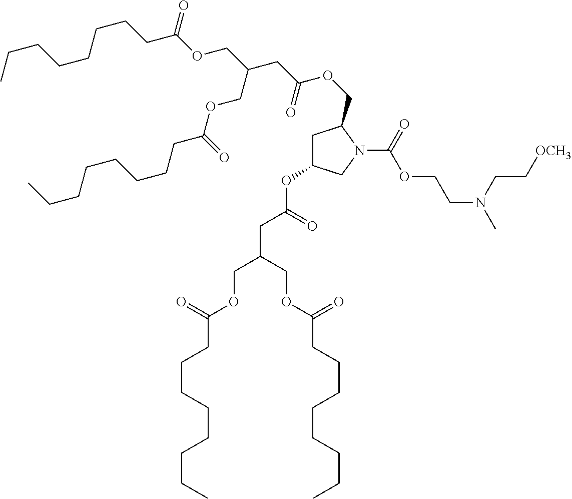 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)CCOC] | 7.55 |
| 115 | CICL-207-115 | |
| 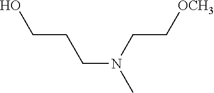 | 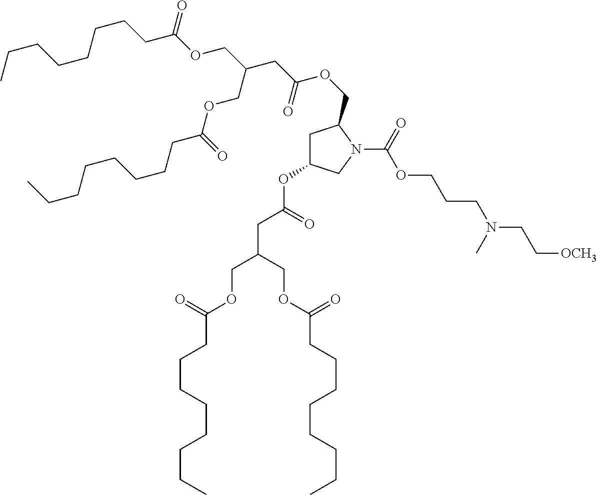 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN(C)CCOC] | 8.37 |
| 116 | CICL-207-116 | |
| 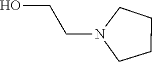 | 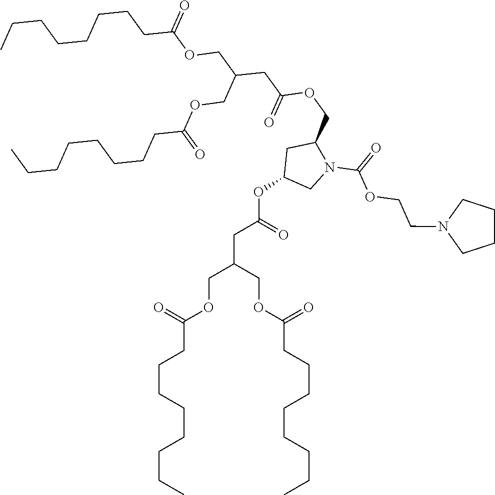 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCCC1] | 9.43 |
| 117 | CICL-207-117 | |
| 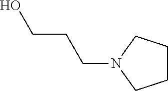 | 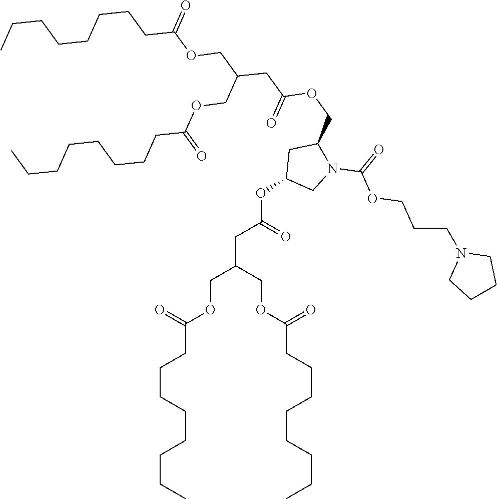 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCCC1] | 10.12 |
| 118 | CICL-207-118 | |
| 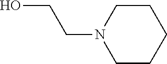 | 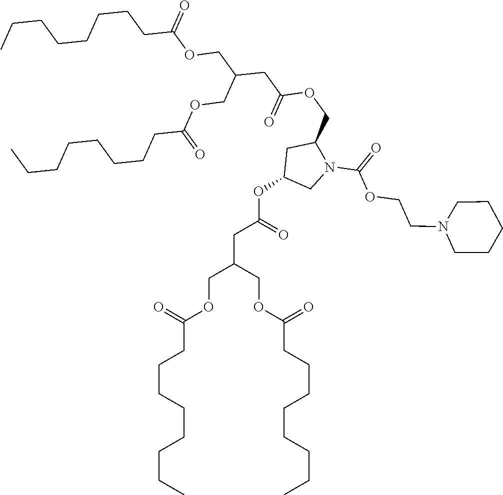 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCCCC1] | 8.44 |
| 119 | CICL-207-119 | |
| 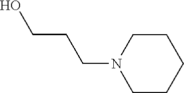 | 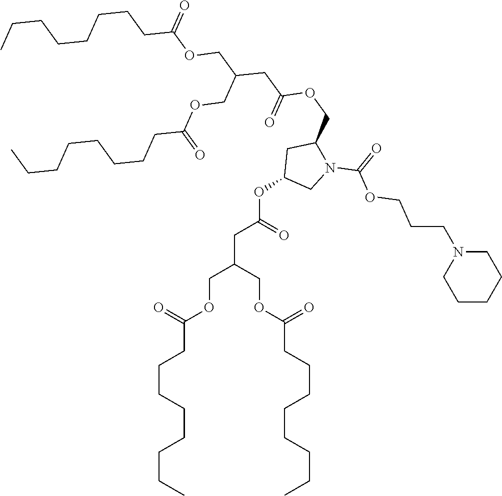 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCCCC1] | 9.21 |
| 120 | CICL-207-120 | |
| 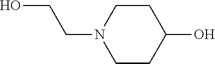 | 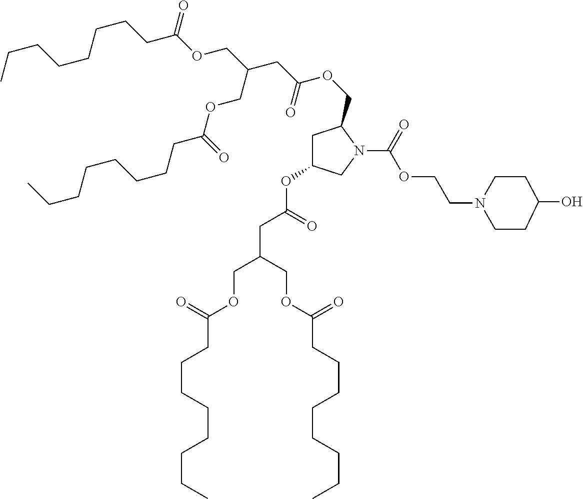 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCC(O)CC1] | 7.77 |
| 121 | CICL-207-121 | |
| 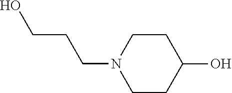 [SMILES: OCCCN1CCC(O)CC1] | 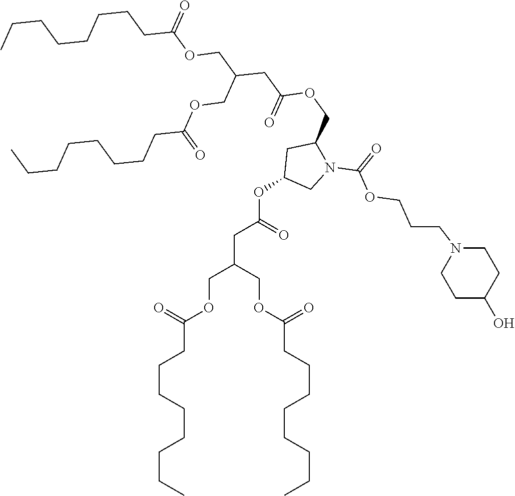 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCC(O)CC1] | 8.38 |
| 122 | CICL-207-122 | |
| 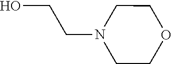 | 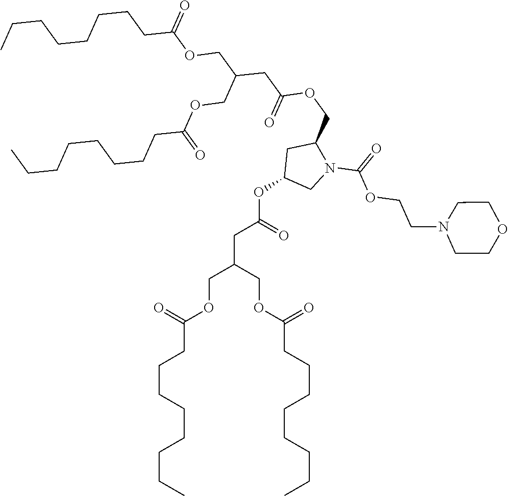 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCOCC1] | 6.17 |
| 123 | CICL-207-123 | |
| 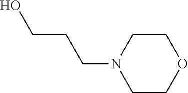 | 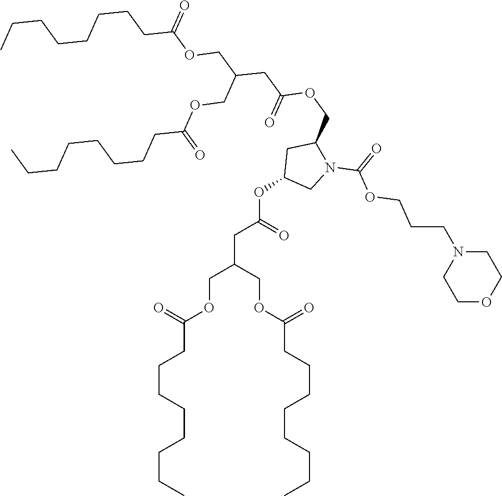 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCOCC1] | 7.04 |
| 124 | CICL-207-124 | |
| 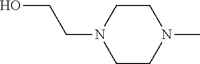 | 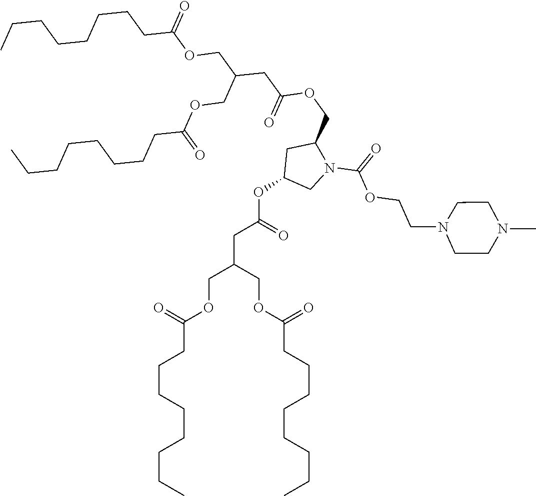 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN1CCN(C)CC1] | 7.56 |
| 125 | CICL-207-125 | |
| 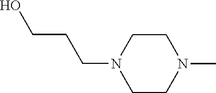 | 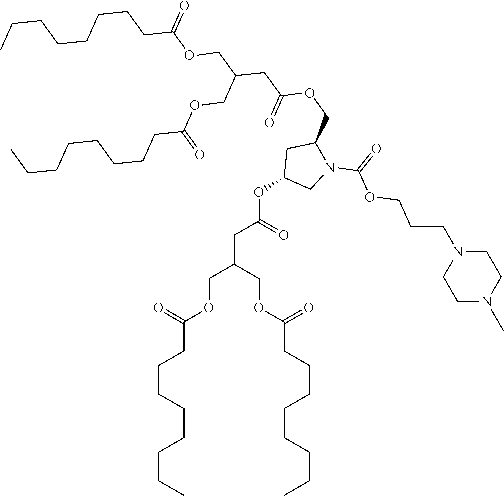 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN1CCN(C)CC1] | 7.64 |
| 126 | CICL-207-126 | |
| 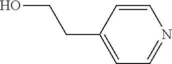 | 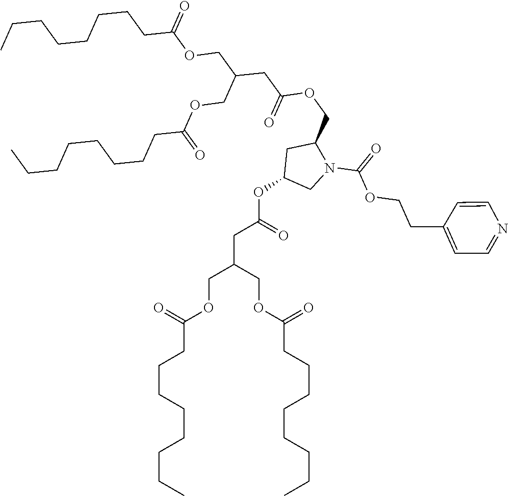 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCc1ccncc1] | 5.64 |
| 127 | CICL-207-127 | |
| 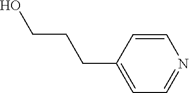 | 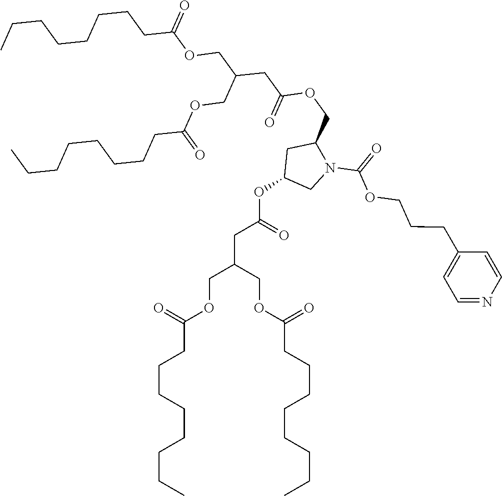 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCc1ccncc1] | 5.93 |
| 128 | CICL-207-128 | |
| 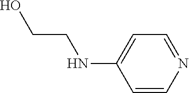 | 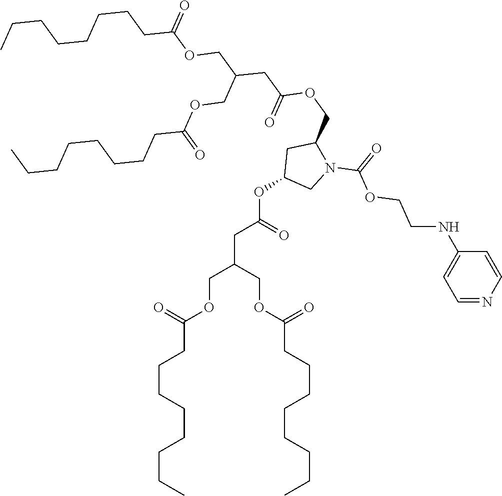 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCNc1ccncc1] | 7.90 |
| 129 | CICL-207-129 | |
| 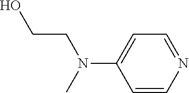 | 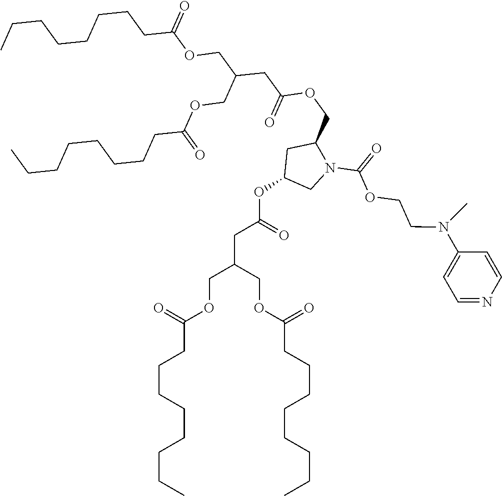 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)c1ccncc1] | 8.65 |
| 130 | CICL-207-130 | |
| 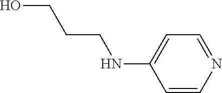 [SMILES: OCCCNc1ccncc1] | 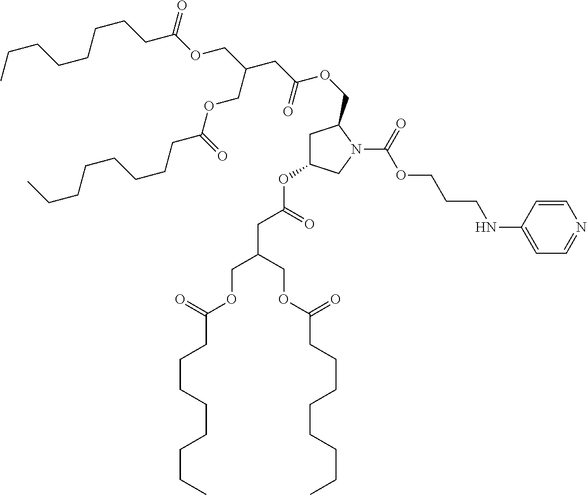 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCNc1ccncc1] | 8.14 |
| 131 | CICL-207-131 | |
| 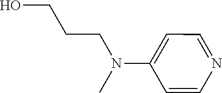 | 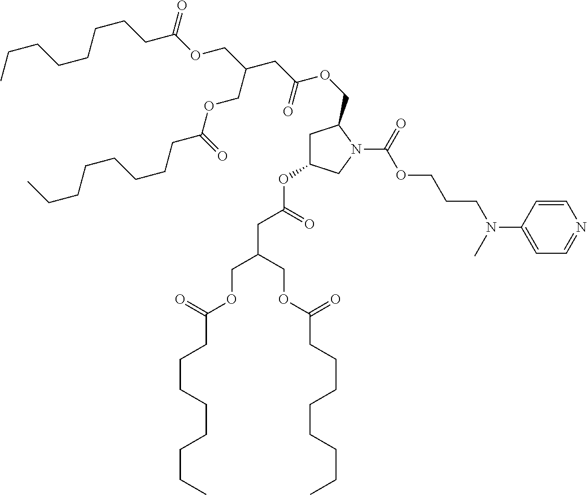 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCCN(C)c1ccncc1] | 9.18 |
| 132 | CICL-207-132 | |
| 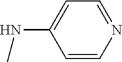 | 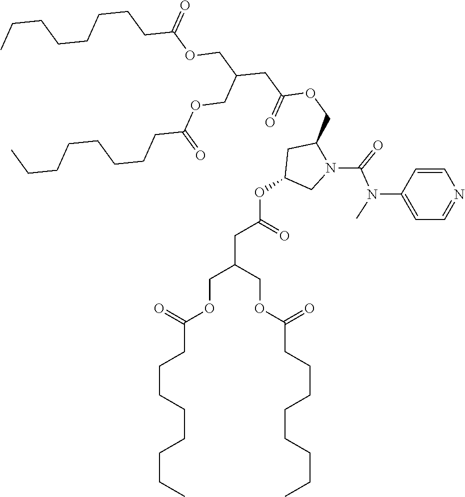 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)N(C)c1ccncc1] | 7.33 |
| 133 | CICL-207-133 | |

The measured pKa data associated with CICL-207 in Table 9 and the calculated pKa data for CICL-207-108 through CICL-207-133 enables the prediction that the combinations CICL-207-109, CICL-207-116, CICL-207-119, CICL-207-122 and CICL-207-130 should have measured pKa data in formulation close to the desired pKa=6-7 midpoint.

In order to move the measured pKa of lipids which result from the combination of acylimidazolides 7, 12, 20, 24, 28, 32, 36, 40, 44, 48, 53 to target the midpoint of the measured pKa range of 6-7 when combined with the X groups 108-133, the c-pKa from Table 5 and the measured pKa data from Tables 8, 9, and 10 will inform the choices made. Acylimidazolides 7, 20, 28, 32, and 53 (used in the synthesis of CICL-221, CICL-224, CICL-223, CICL-216, and CICL-238, respectively) will be combined with X groups: 116, 119, 122, and 131. Acylimidazolides 12, 24, and 36 (used in the synthesis of CICL-222, CICL-225, and CICL-215, respectively) will approach the pKa 6-7 midpoint when combined with X groups 109, 116, 119, 122, and 130. The combination of acylimidazolide 40 (used in the synthesis of CICL-220) with X groups 109, 116, 122, and 130 will approach the mid-point target, as will the concatenation of acylimidazoides 44 and 48 (used in the synthesis of CICL-219 and CICL-218, respectively) with X groups: 109, 119, and 130. Lipids previously derived from acylimidazolides 57, 61, 65, 69, 76, 80, 85 have not been associated with measured pKa values. These acylimidazolides will be combined with head groups 109, 116, 119, 122, and 130 to target mid-range (pKa 6-7) lipids. The structures of the ionizable cationic lipids that result from the combinations cited above are presented in Table 6.

TABLE 6
Lipids formed from the Combination of Core Acylimidazolide 7, 12, 20, 24, 28, 32,
36, 40, 44, 48, 53 with head groups selected from 108 through 133.

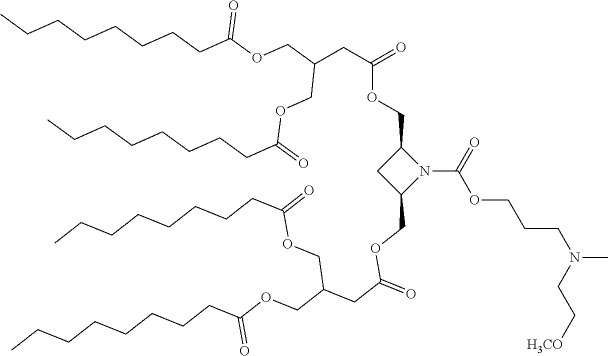 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OCCCN(C)CCOC]

CICL-221-116

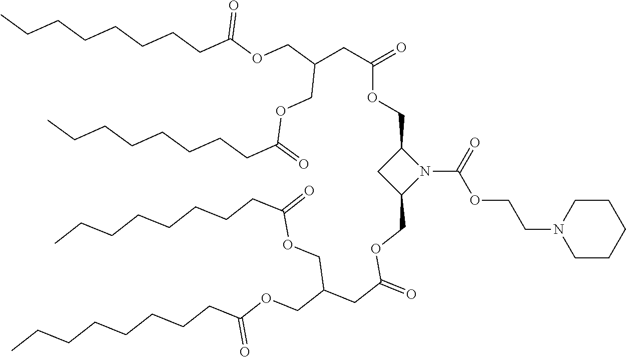 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OCCN1CCCCC1]

CICL-221-119

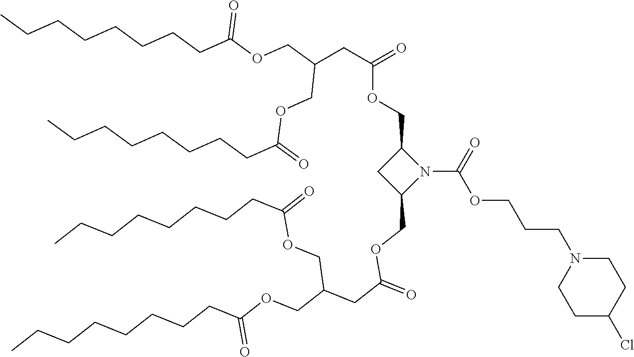 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OCCCN1CCC(Cl)CC1]

CICL-221-122

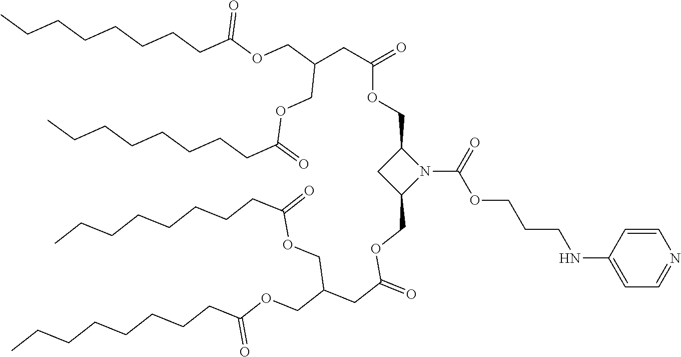 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OCCCNc1ccncc1]

CICL-221-131

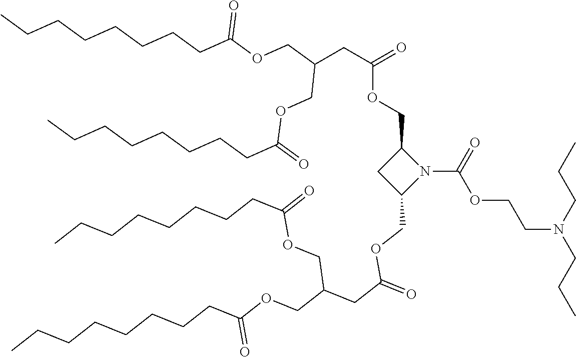 [SMILES: CCCCCCCCC(=O)OCC(COC(=O)CCCCCCCC)CC(=O)OC[C@@H]1C[C@@H](COC(=O)CC(COC(=O)CCCCCCCC)COC(=O)CCCCCCCC)N1C(=O)OCCN(CCC)CCC]

CICL-222-109

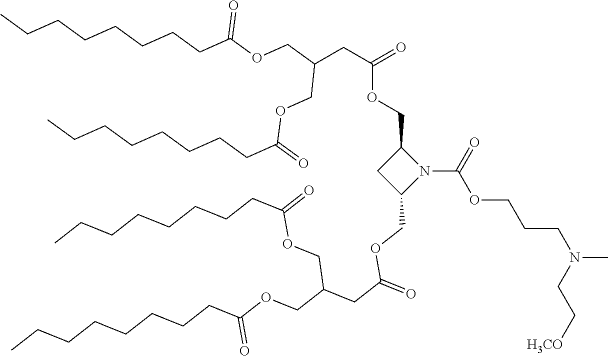 [SMILES: CCCCCCCCC(=O)OCC(COC(=O)CCCCCCCC)CC(=O)OC[C@@H]1C[C@@H](COC(=O)CC(COC(=O)CCCCCCCC)COC(=O)CCCCCCCC)N1C(=O)OCCCN(C)CCOC]

CICL-222-116

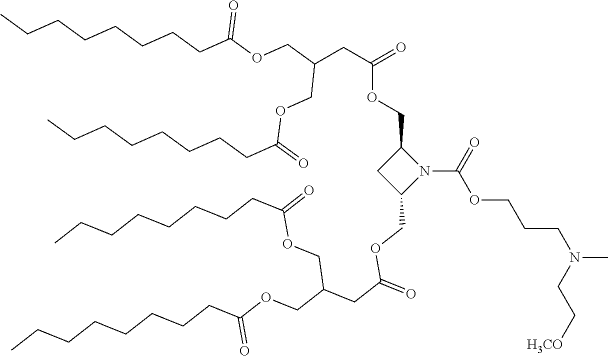 [SMILES: CCCCCCCCC(=O)OCC(COC(=O)CCCCCCCC)CC(=O)OC[C@@H]1C[C@@H](COC(=O)CC(COC(=O)CCCCCCCC)COC(=O)CCCCCCCC)N1C(=O)OCCCN(C)CCOC]

CICL-222-119

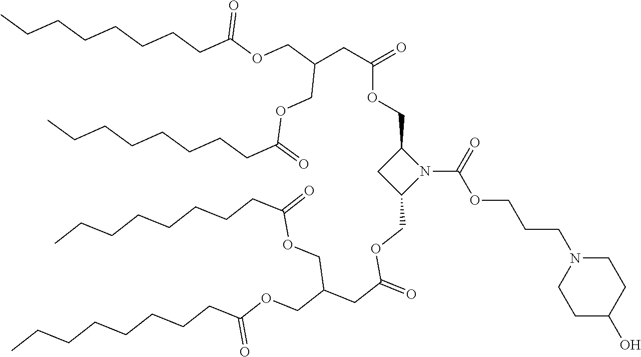 [SMILES: CCCCCCCCC(=O)OCC(COC(=O)CCCCCCCC)CC(=O)OC[C@@H]1C[C@@H](COC(=O)CC(COC(=O)CCCCCCCC)COC(=O)CCCCCCCC)N1C(=O)OCCCN1CCC(O)CC1]

CICL-222-122

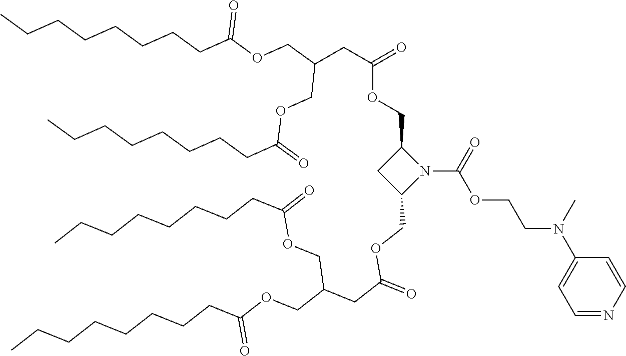 [SMILES: CCCCCCCCC(=O)OCC(COC(=O)CCCCCCCC)CC(=O)OC[C@@H]1C[C@@H](COC(=O)CC(COC(=O)CCCCCCCC)COC(=O)CCCCCCCC)N1C(=O)OCCN(C)c1ccncc1]

CICL-222-130

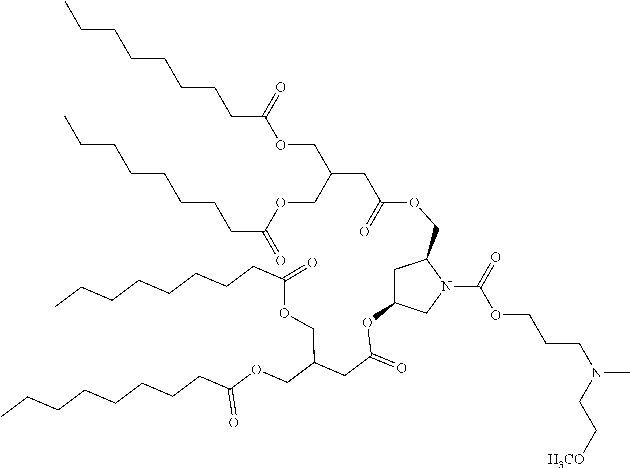 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OCCCN(C)CCOC]

CICL-224-116

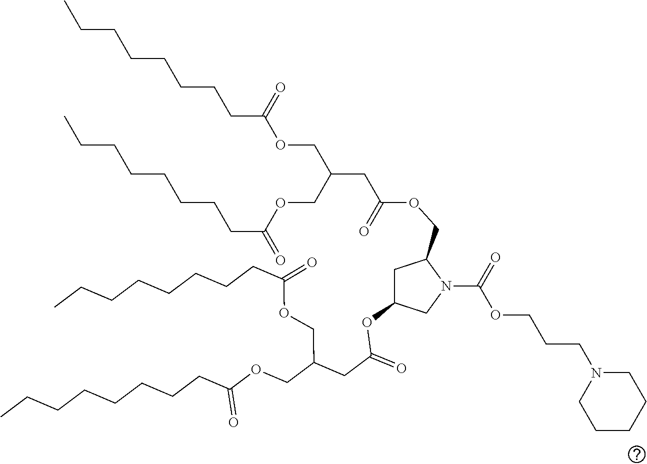 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OCCCN1CCCCC1]

CICL-224-119

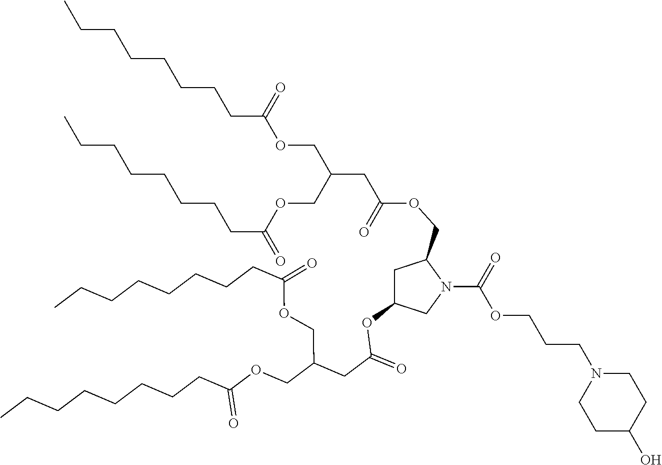 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OCCCN1CCC(O)CC1]

CICL-224-122

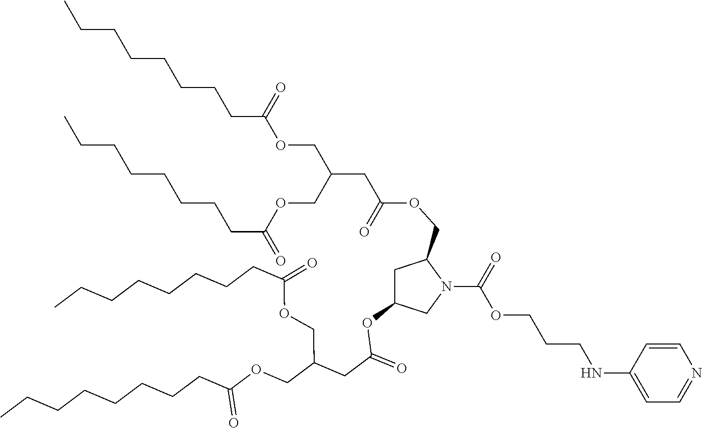 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OCCCNc1ccncc1]

CICL-224-131

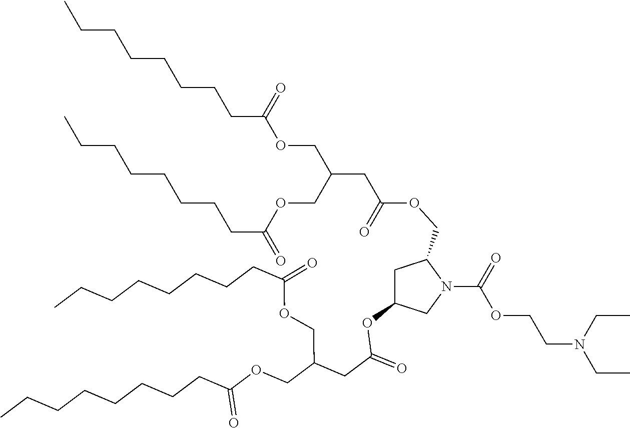 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCN(CC)CC]

CICL-225-109

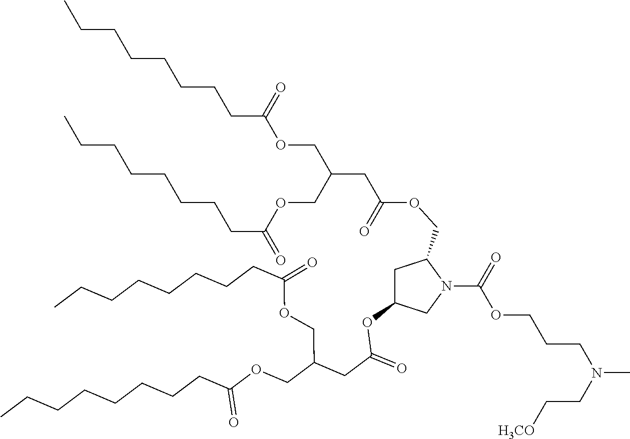 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCCN(C)CCOC]

CICL-225-116

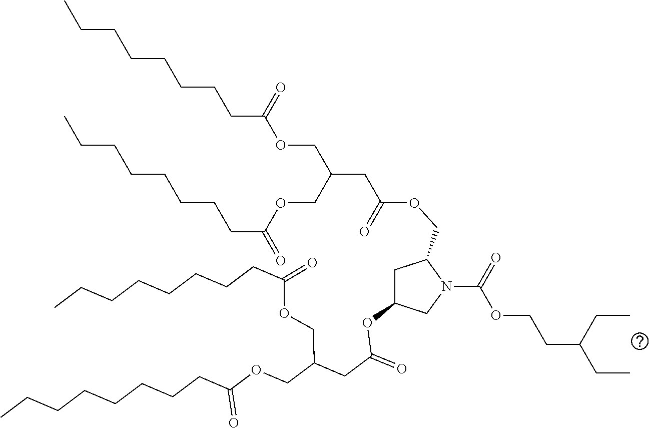 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCC(CC)CC]

CICL-225-119

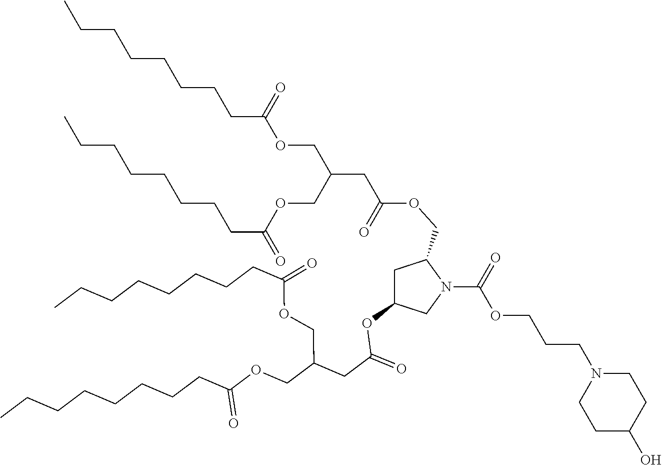 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCCN1CCC(O)CC1]

CICL-225-122

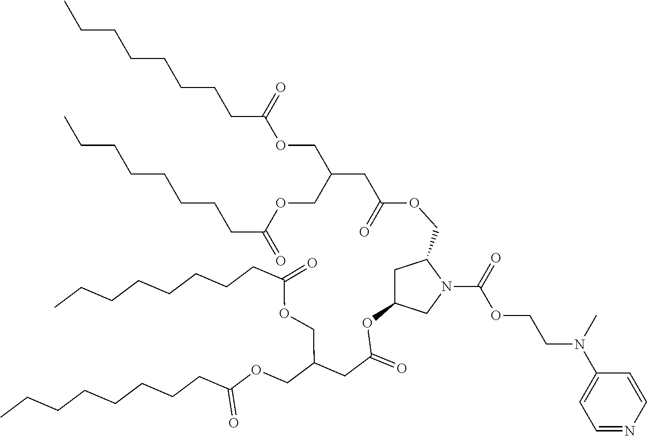 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCN(C)c1ccncc1]

CICL-225-130

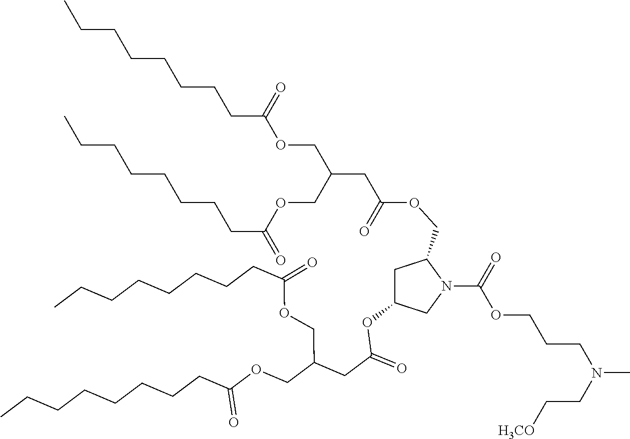 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OCCCN(C)CCOC]

CICL-223-116

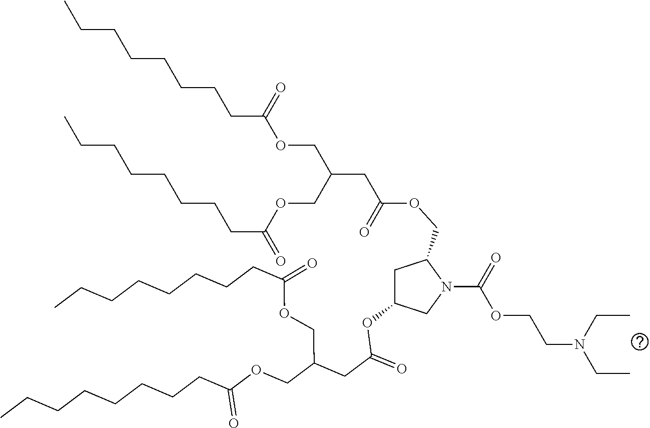 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OCCN(CC)CC]

CICL-223-119

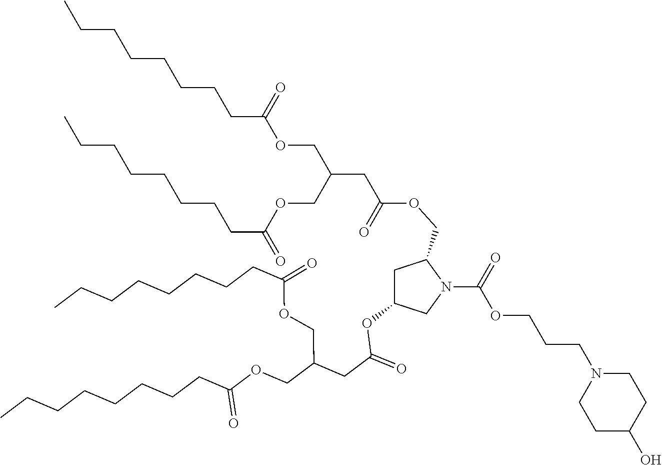 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OCCCN1CCC(O)CC1]

CICL-223-122

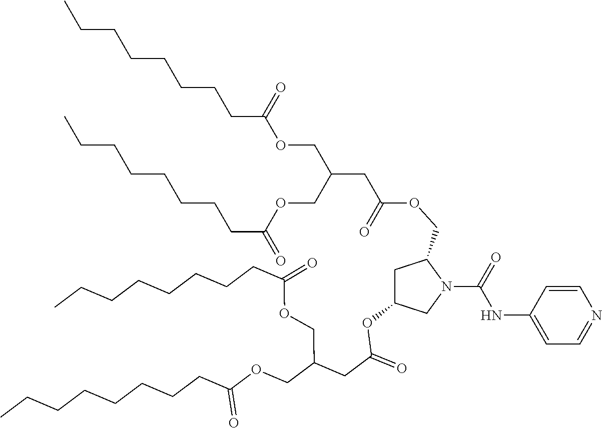 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)Nc1ccncc1]

CICL-223-131

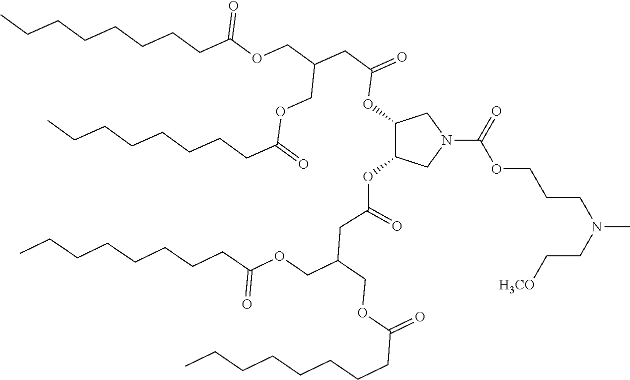 [SMILES: CCCCCCCCC(=O)OCC(COC(=O)CCCCCCCC)CC(=O)O[C@H]1CN(C(=O)OCCCN(C)CCOC)C[C@H]1OC(=O)CC(COC(=O)CCCCCCCC)COC(=O)CCCCCCCC]

CICL-216-116

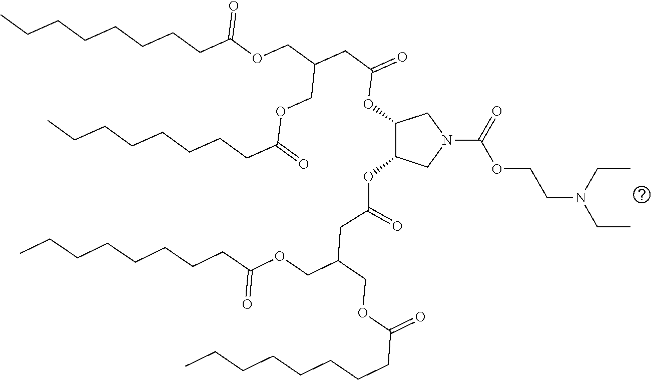 [SMILES: CCCCCCCCC(=O)OCC(COC(=O)CCCCCCCC)CC(=O)O[C@H]1CN(C(=O)OCCN(CC)CC)C[C@H]1OC(=O)CC(COC(=O)CCCCCCCC)COC(=O)CCCCCCCC]

CICL-216-119

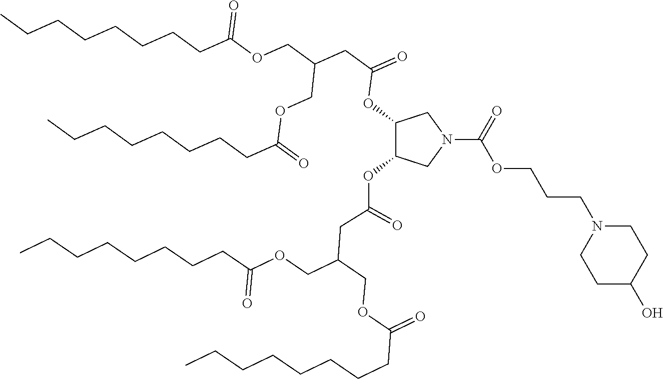 [SMILES: CCCCCCCCC(=O)OCC(COC(=O)CCCCCCCC)CC(=O)O[C@H]1CN(C(=O)OCCCN2CCC(O)CC2)C[C@H]1OC(=O)CC(COC(=O)CCCCCCCC)COC(=O)CCCCCCCC]

CICL-216-122

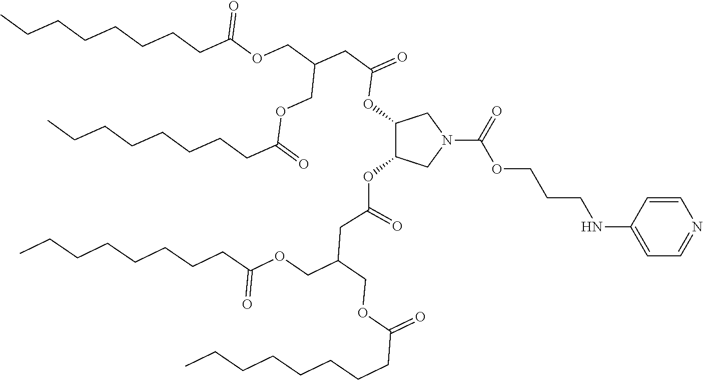 [SMILES: CCCCCCCCC(=O)OCC(COC(=O)CCCCCCCC)CC(=O)O[C@H]1CN(C(=O)OCCCNc2ccncc2)C[C@H]1OC(=O)CC(COC(=O)CCCCCCCC)COC(=O)CCCCCCCC]

CICL-216-131

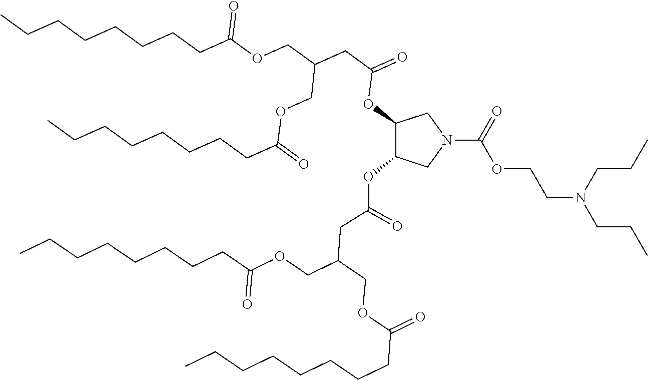 [SMILES: CCCCCCCCC(=O)OCC(COC(=O)CCCCCCCC)CC(=O)O[C@H]1CN(C(=O)OCCN(CCC)CCC)C[C@@H]1OC(=O)CC(COC(=O)CCCCCCCC)COC(=O)CCCCCCCC]

CICL-215-109

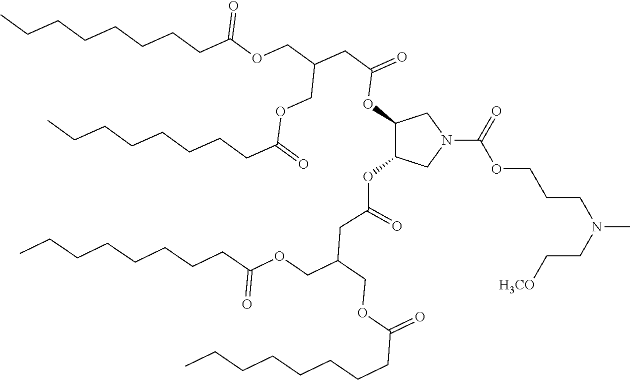 [SMILES: CCCCCCCCC(=O)OCC(COC(=O)CCCCCCCC)CC(=O)O[C@H]1CN(C(=O)OCCCN(C)CCOC)C[C@@H]1OC(=O)CC(COC(=O)CCCCCCCC)COC(=O)CCCCCCCC]

CICL-215-116

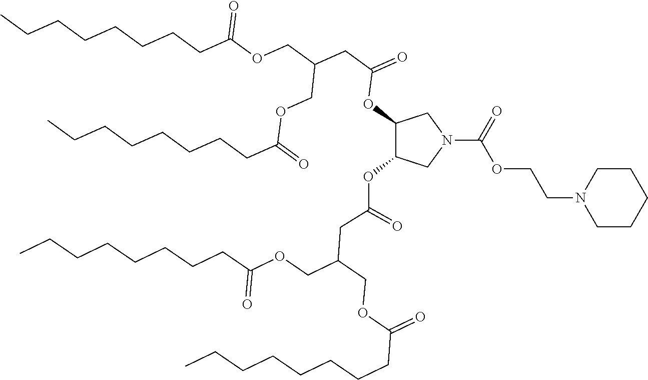 [SMILES: CCCCCCCCC(=O)OCC(COC(=O)CCCCCCCC)CC(=O)O[C@H]1CN(C(=O)OCCN2CCCCC2)C[C@@H]1OC(=O)CC(COC(=O)CCCCCCCC)COC(=O)CCCCCCCC]

CICL-215-119

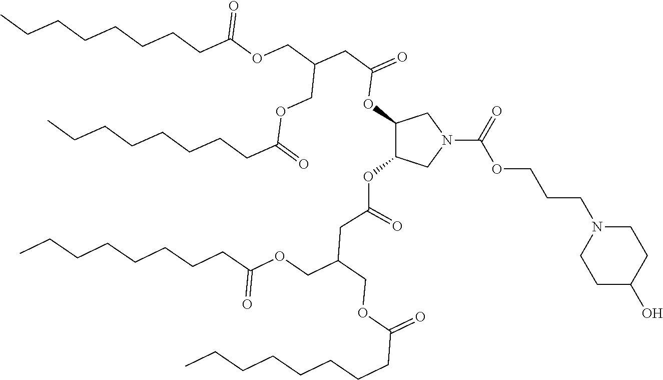 [SMILES: CCCCCCCCC(=O)OCC(COC(=O)CCCCCCCC)CC(=O)O[C@H]1CN(C(=O)OCCCN2CCC(O)CC2)C[C@@H]1OC(=O)CC(COC(=O)CCCCCCCC)COC(=O)CCCCCCCC]

CICL-215-122

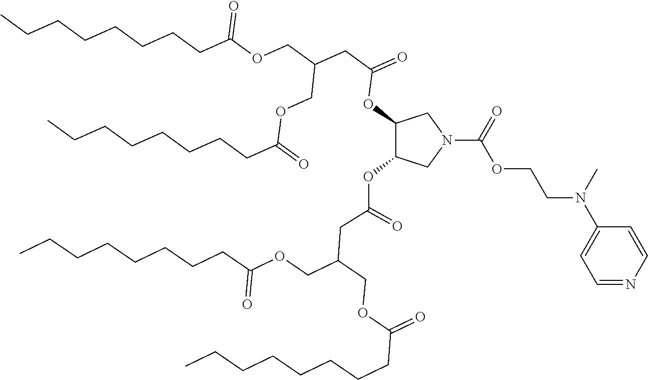 [SMILES: CCCCCCCCC(=O)OCC(COC(=O)CCCCCCCC)CC(=O)O[C@H]1CN(C(=O)OCCN(C)c2ccncc2)C[C@@H]1OC(=O)CC(COC(=O)CCCCCCCC)COC(=O)CCCCCCCC]

CICL-215-130

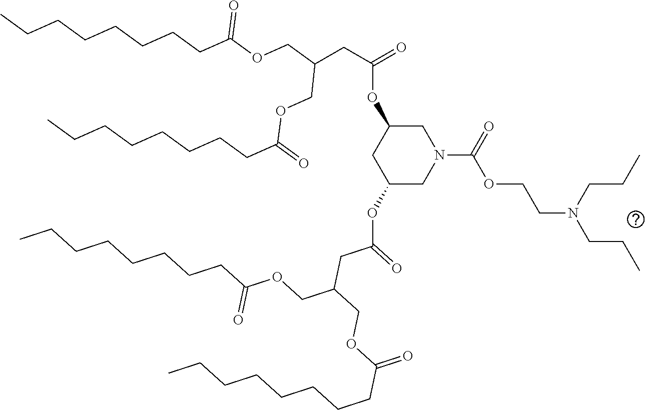 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCCN(CCC)CCC)C1]

CICL-220-109

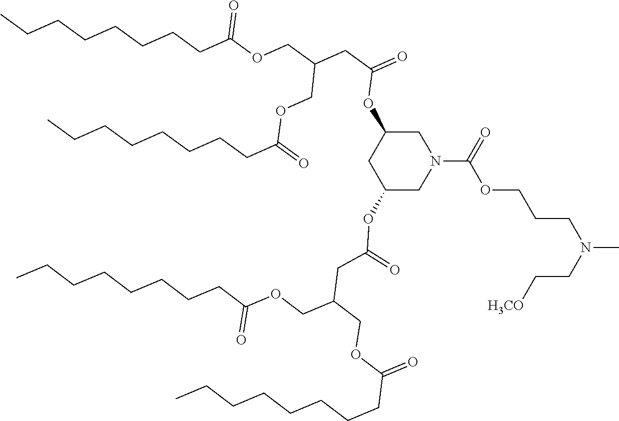 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCCCN(C)CCOC)C1]

CICL-220-116

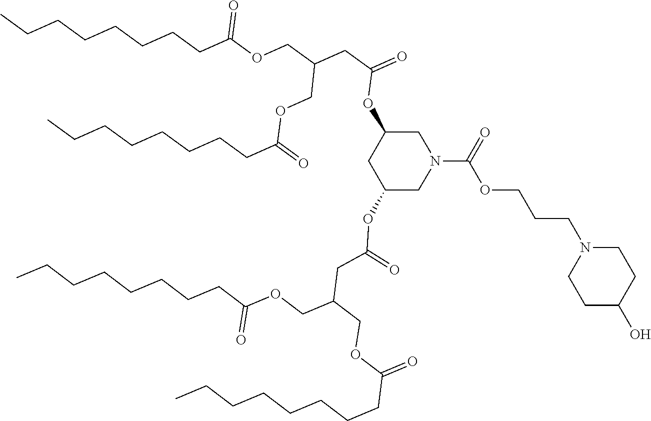 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCCCN2CCC(O)CC2)C1]

CICL-220-122

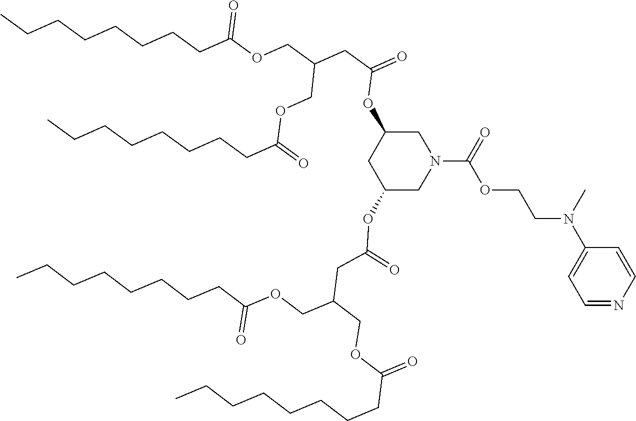 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCCN(C)c2ccncc2)C1]

CICL-220-130

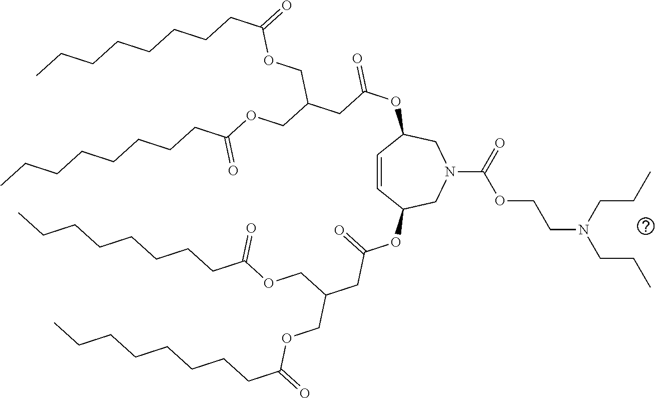 [SMILES: CCCCCCCCC(=O)OCC(COC(=O)CCCCCCCC)CC(=O)O[C@@H]1C=C[C@H](OC(=O)CC(COC(=O)CCCCCCCC)COC(=O)CCCCCCCC)CN(C(=O)OCCN(CCC)CCC)C1]

CICL-219-109

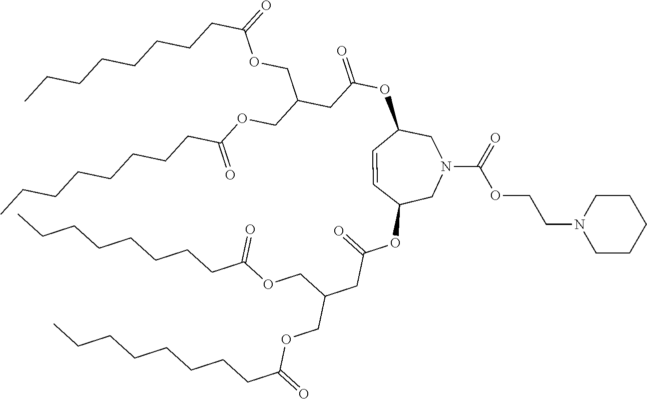

CICL-219-119

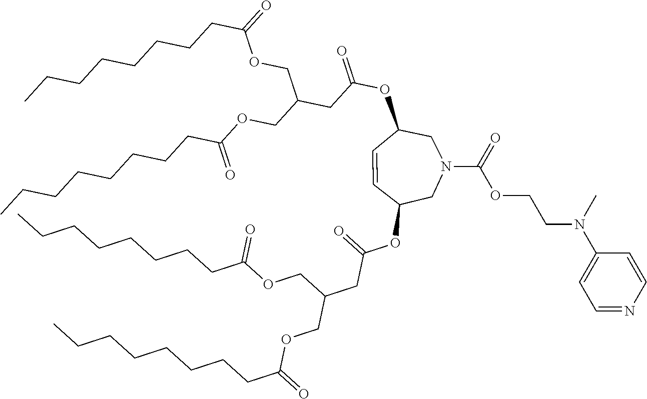

CICL-219-130

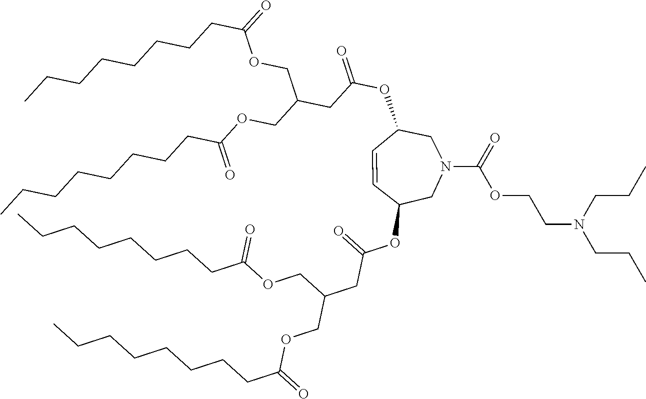

CICL-218-109

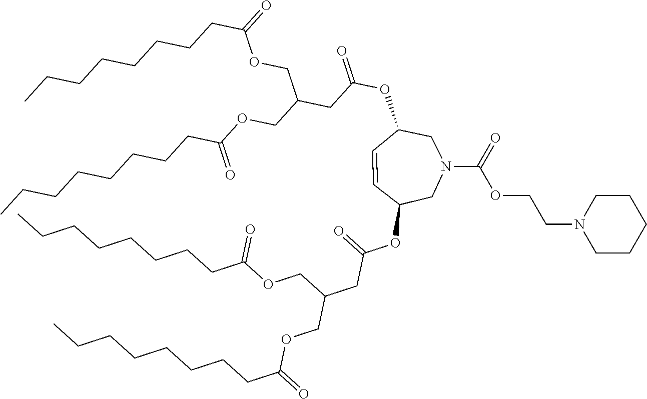

CICL-218-119

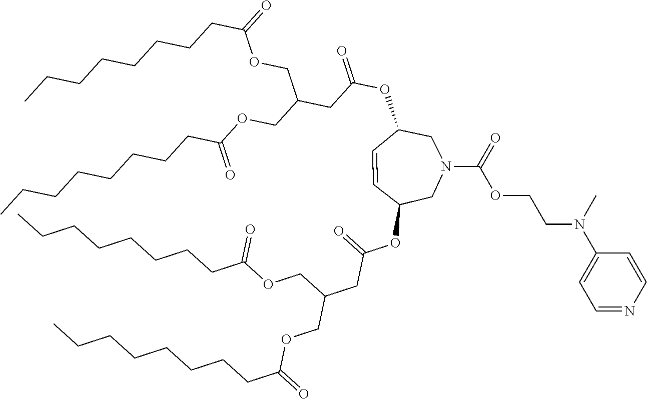

CICL-219-130

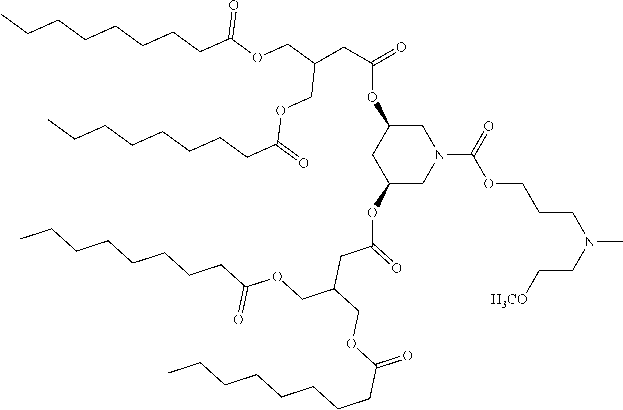 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OCCCN(C)CCOC)C1]

CICL-238-116

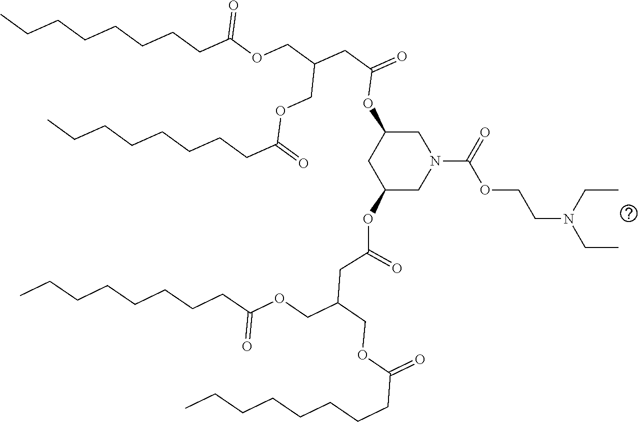 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OCCN(CC)CC)C1]

CICL-238-119

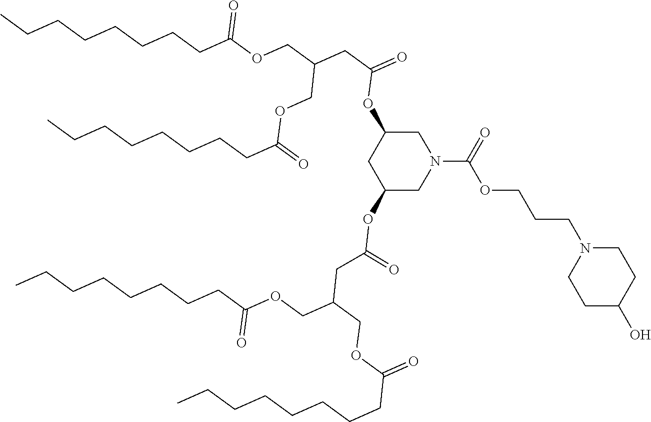 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OCCCN2CCC(O)CC2)C1]

CICL-238-122

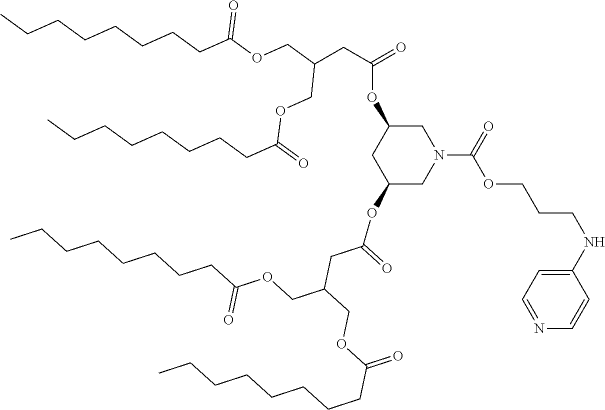 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OCCCNc2ccncc2)C1]

CICL-238-131

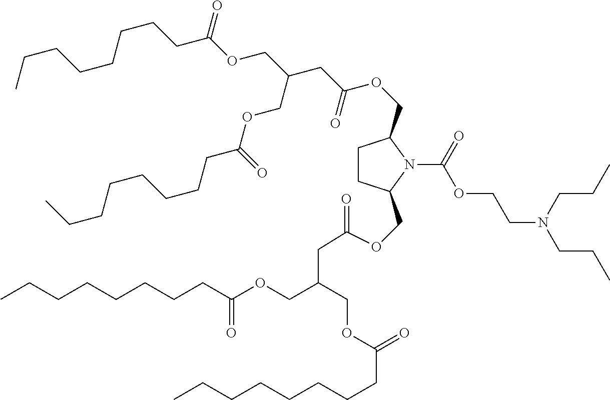 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCN(CCC)CCC]

CICL-242-109

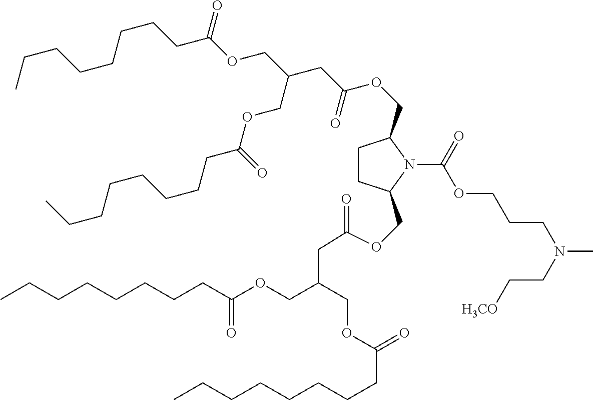 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCCN(C)CCOC]

CICL-242-116

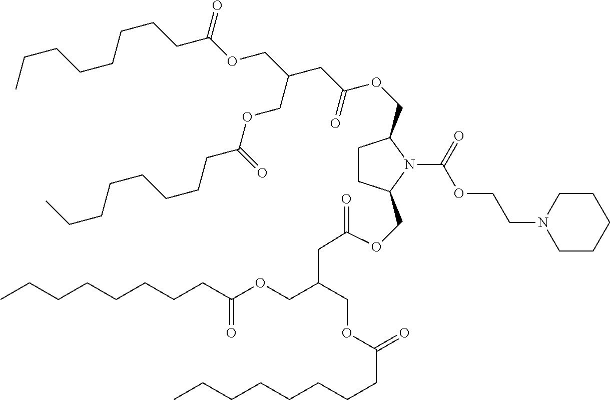 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCN1CCCCC1]

CICL-242-119

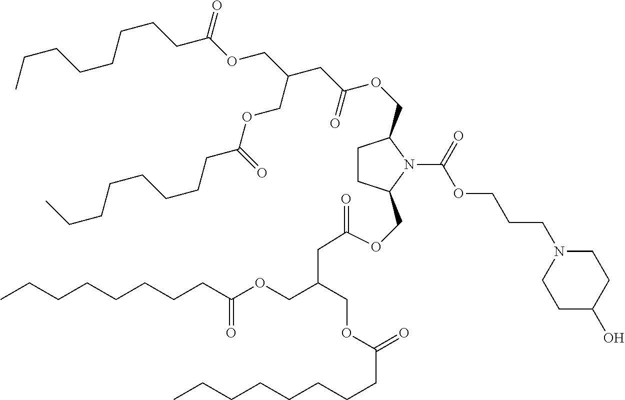 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCCN1CCC(O)CC1]

CICL-242-122

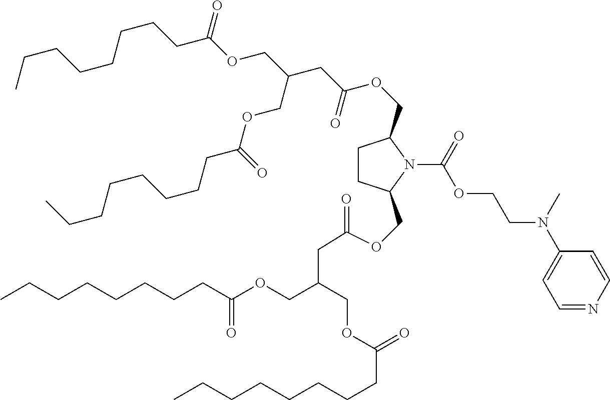 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCN(C)c1ccncc1]

CICL-242-130

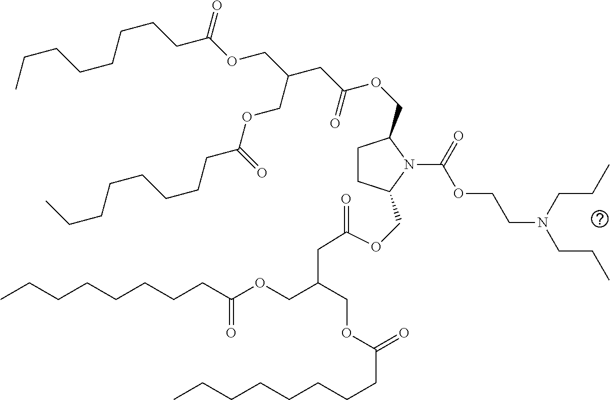 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCN(CCC)CCC]

CICL-243-109

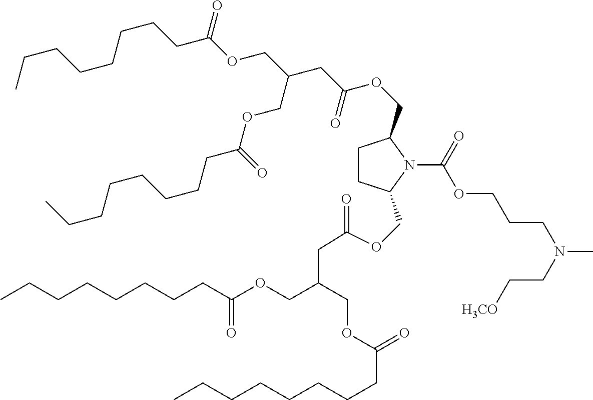 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCCN(C)CCOC]

CICL-242-116

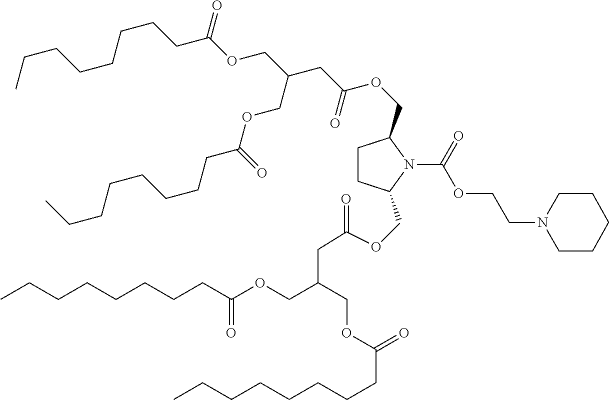 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCN1CCCCC1]

CICL-242-119

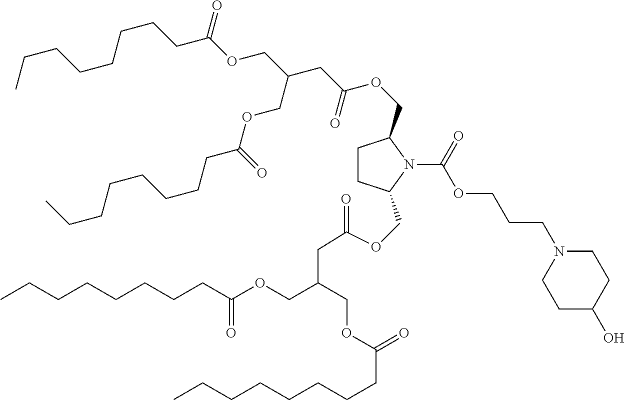 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCCN1CCC(O)CC1]

CICL-242-122

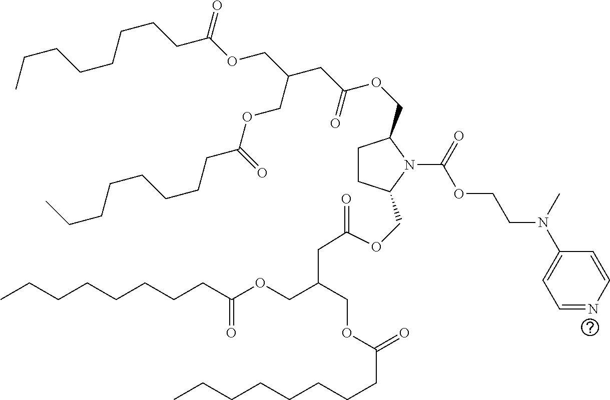 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCN(C)c1ccncc1]

CICL-242-130

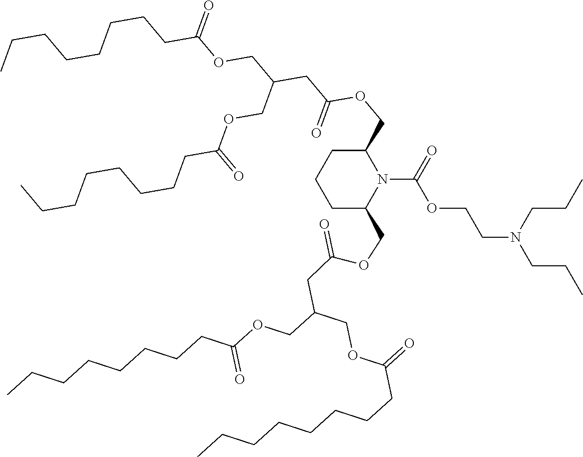 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCN(CCC)CCC]

CICL-244-109

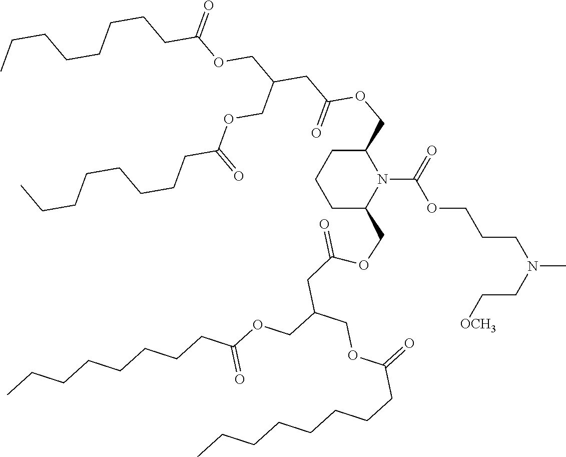 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCCN(C)CCOC]

CICL-244-116

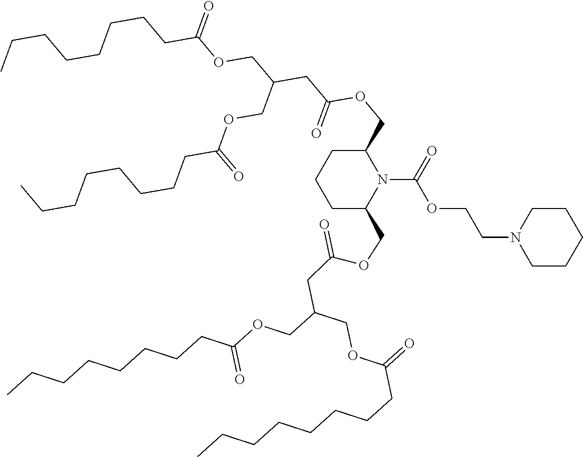 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCN1CCCCC1]

CICL-244-119

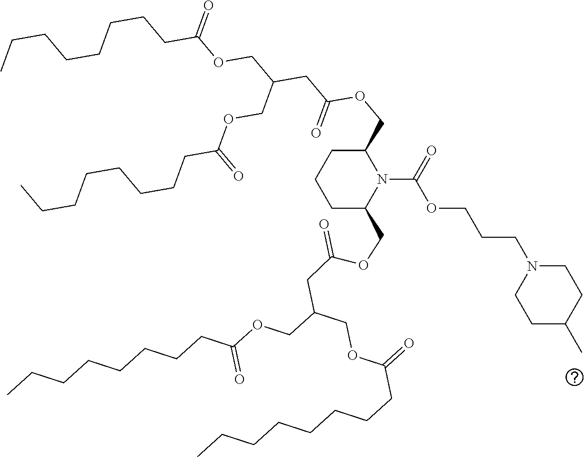 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCCN1CCC(C)CC1]

CICL-244-122

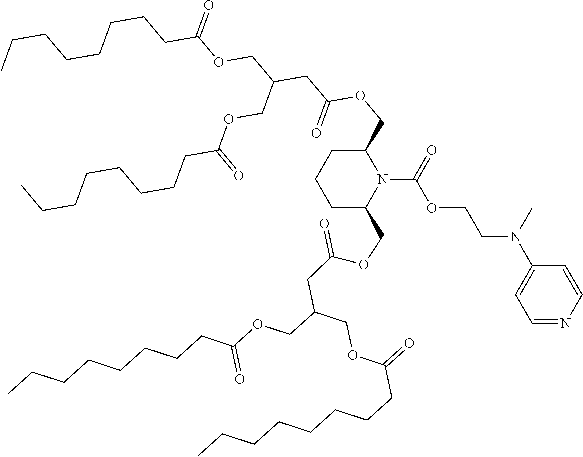 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCN(C)c1ccncc1]

CICL-244-130

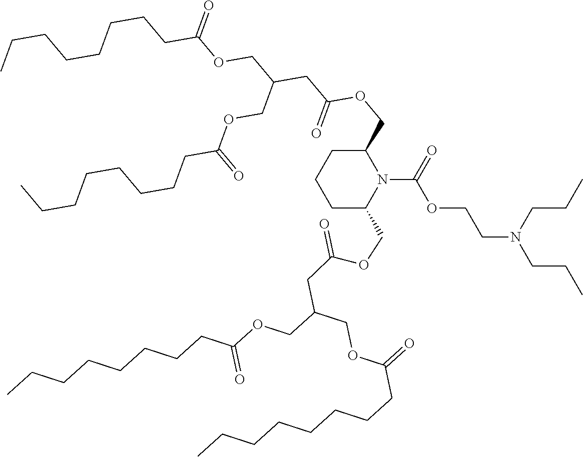 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCN(CCC)CCC]

CICL-245-109

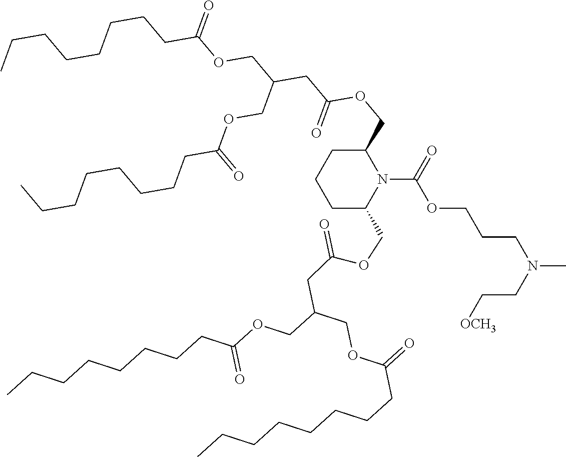 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCCN(C)CCOC]

CICL-245-116

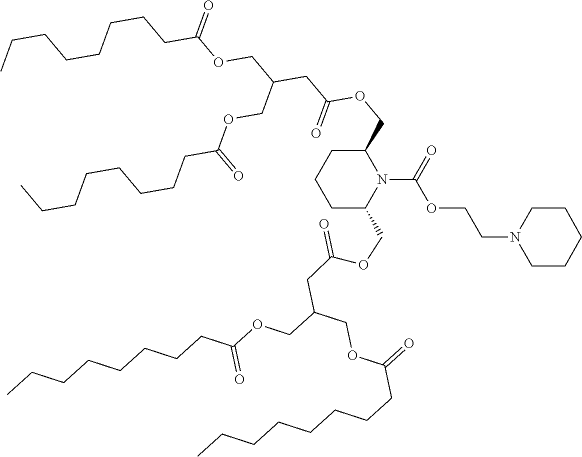 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCN1CCCCC1]

CICL-245-119

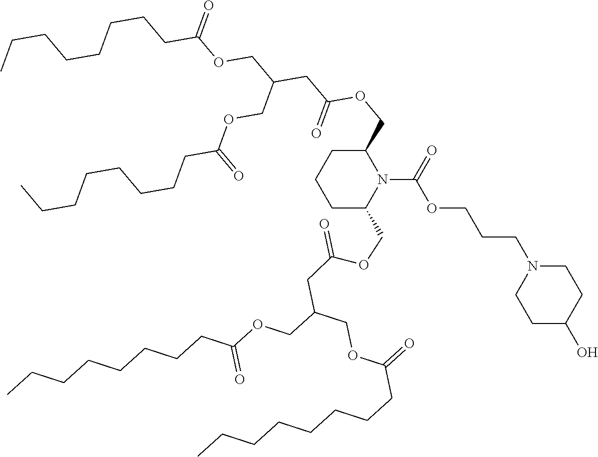 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCCN1CCC(O)CC1]

CICL-245-122

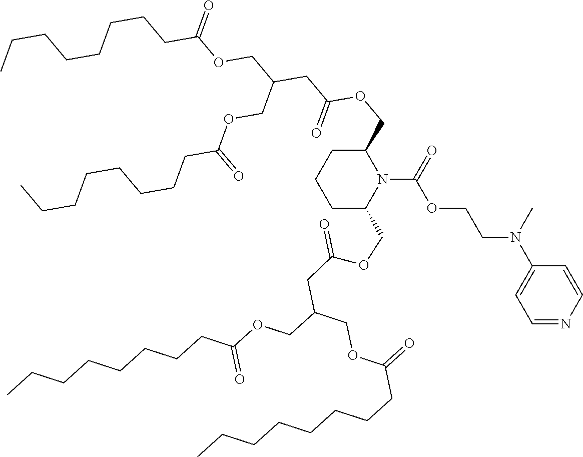 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCN(C)c1ccncc1]

CICL-245-130

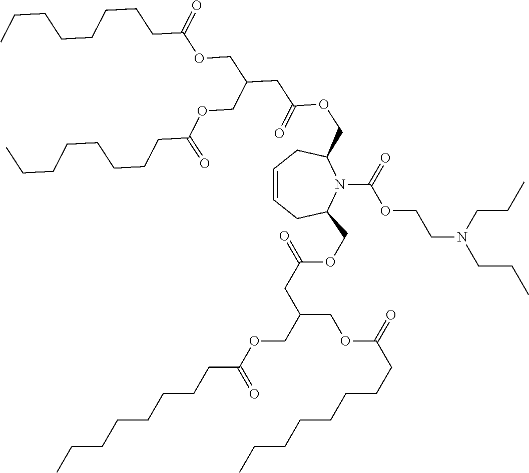 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCCN(CCC)CCC]

CICL-246-109

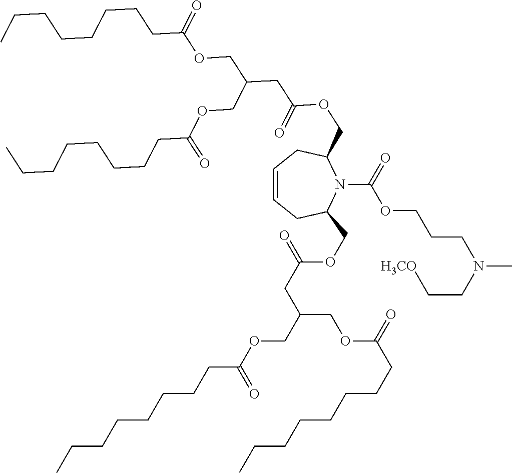 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCCCN(C)CCOC]

CICL-246-116

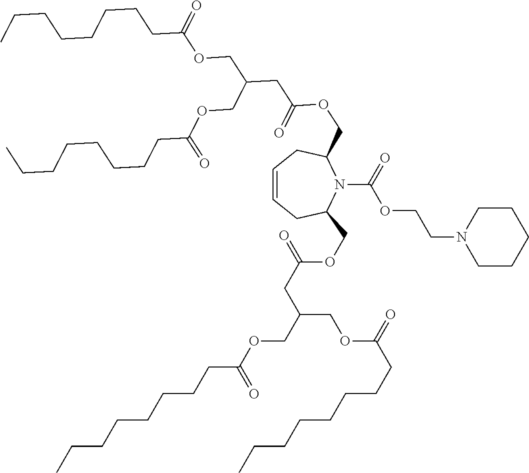 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCCN1CCCCC1]

CICL-246-119

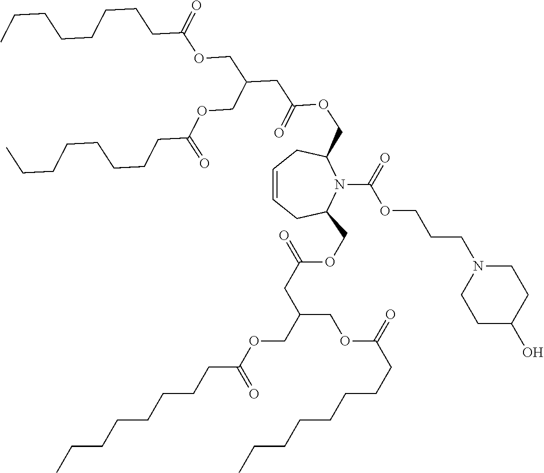 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCCCN1CCC(O)CC1]

CICL-246-122

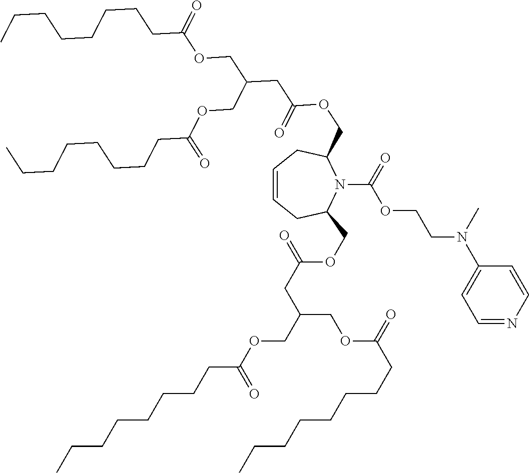 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCCN(C)c1ccncc1]

CICL-246-130

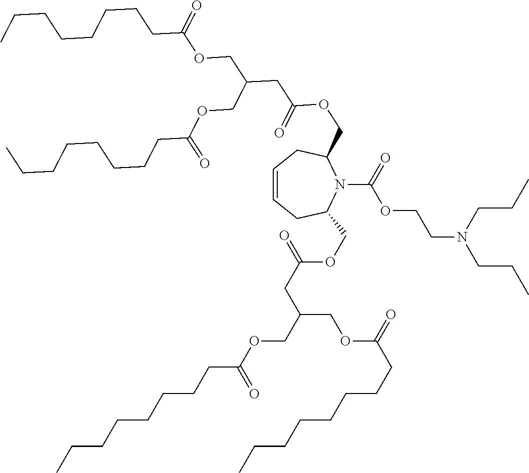 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCCN(CCC)CCC]

CICL-247-109

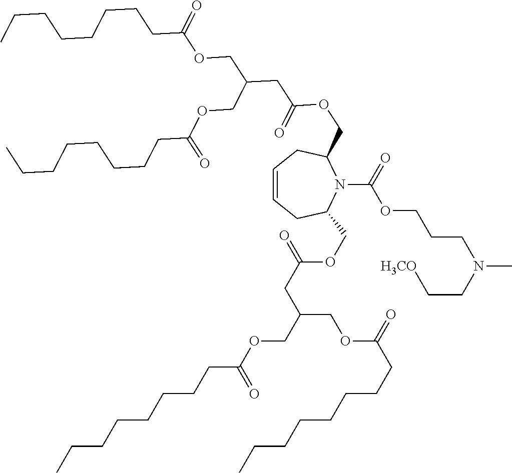 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCCCN(C)CCOC]

CICL-247-116

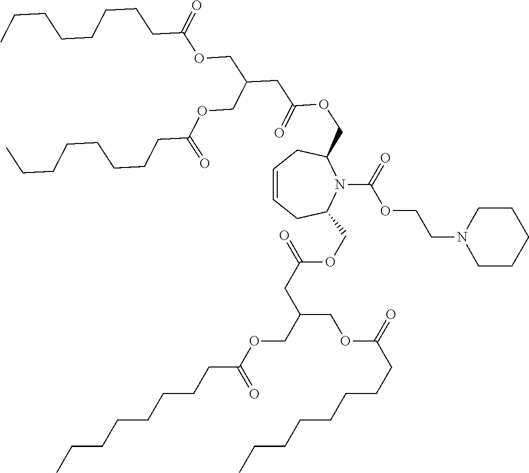 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCCN1CCCCC1]

CICL-247-119

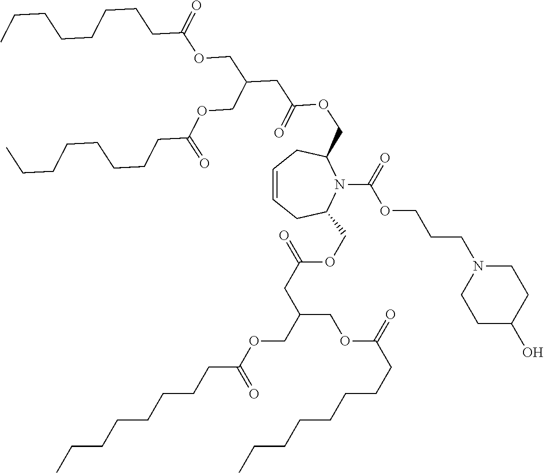 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCCCN1CCC(O)CC1]

CICL-247-122

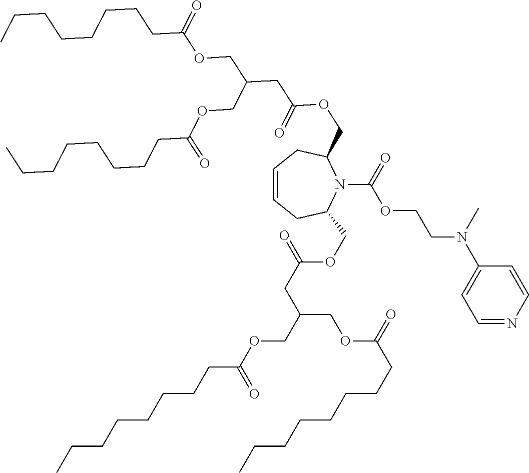 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCCN(C)c1ccncc1]

CICL-247-130

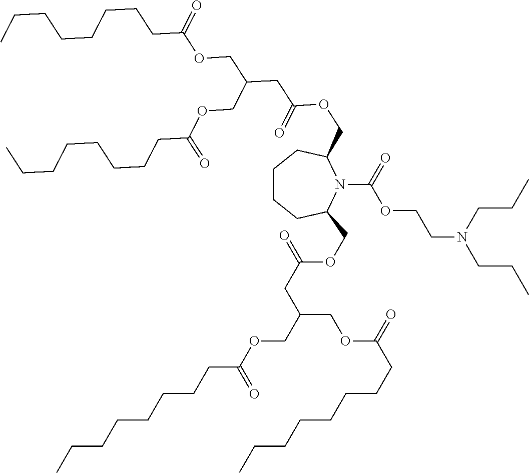 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCCN(CCC)CCC]

CICL-248-109

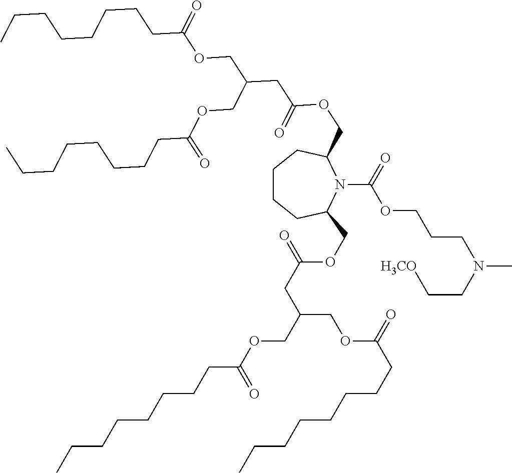 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCCCN(C)CCOC]

CICL-248-116

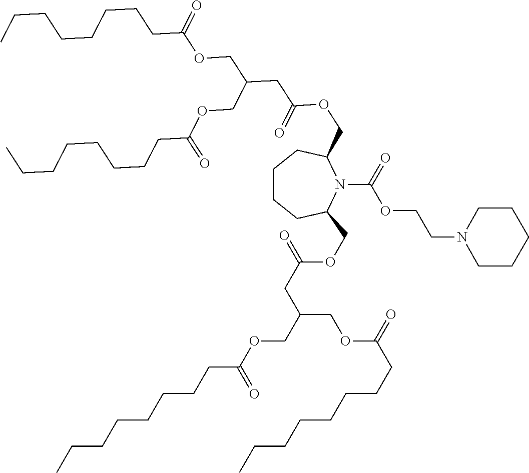 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCCN1CCCCC1]

CICL-248-119

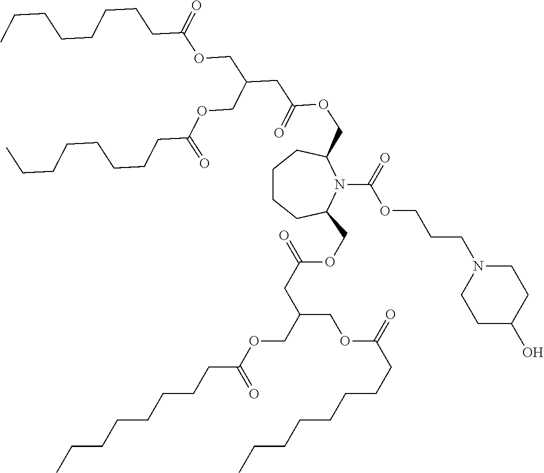 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCCCN1CCC(O)CC1]

CICL-248-122

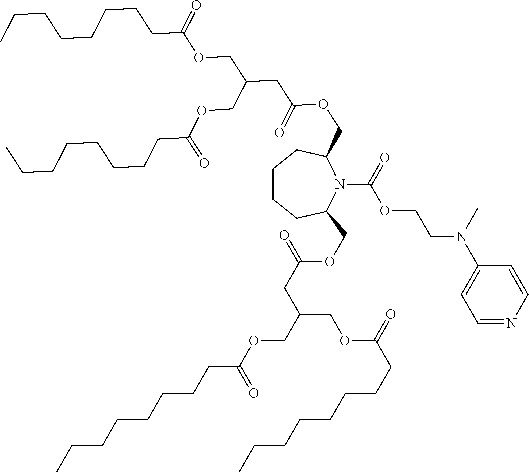 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCCN(C)c1ccncc1]

CICL-248-130

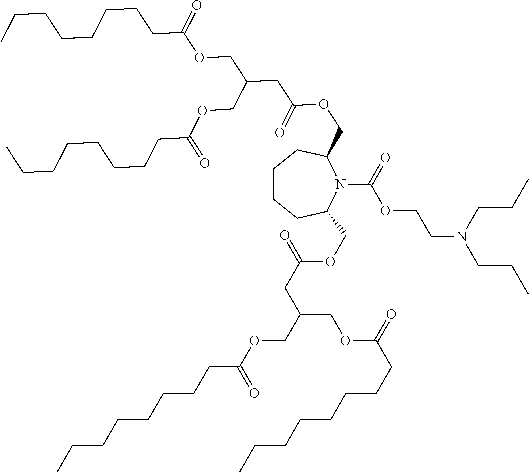 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCN(CCC)CCC]

CICL-249-109

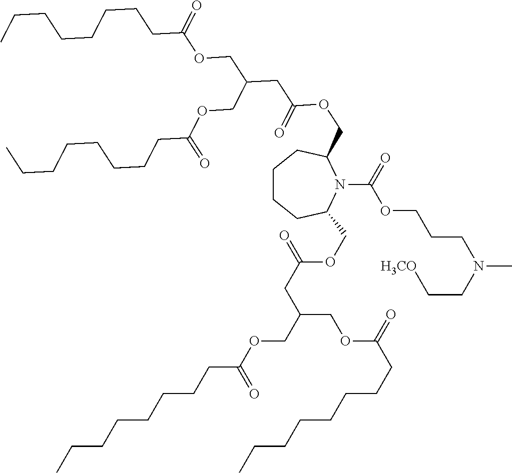 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCCN(C)CCOC]

CICL-249-116

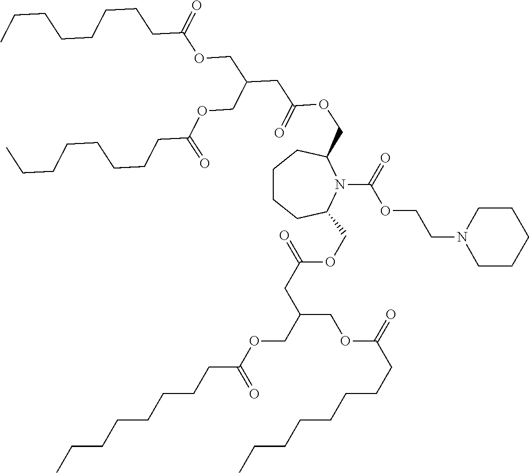 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCN1CCCCC1]

CICL-249-119

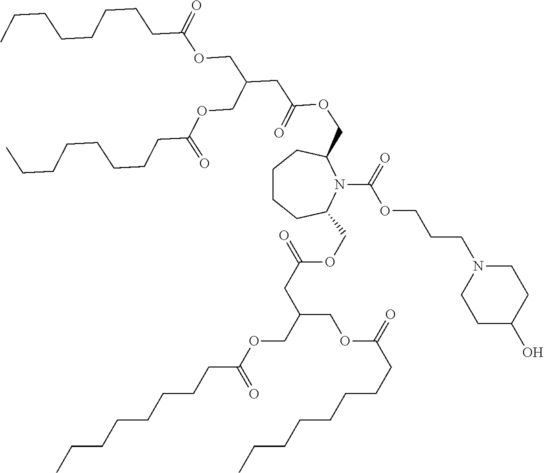 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCCN1CCC(O)CC1]

CICL-249-122

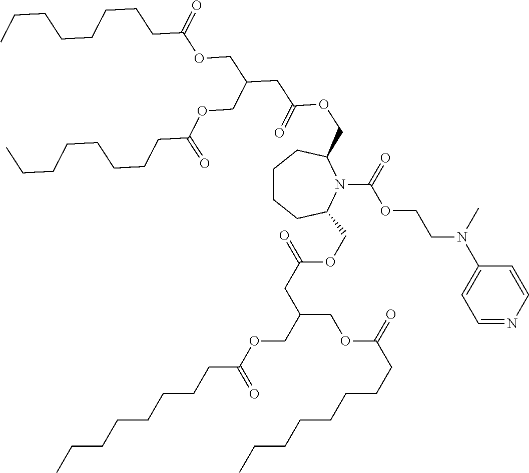 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCN(C)c1ccncc1]

CICL-249-130

The following table provides the specific lipid compositions of the LNPs and tLNPs used in the following Examples (Examples 66, 67, 68, 69, 70, 71, and 72).

TABLE 7
Compositions of LNPs and tLNPs used in Examples 66-72

| | Composition |
|---|---|
| Composition | Code |
| ALC-0315:DSPC:CHOL:DMG-PEG(2k):DSPE-PEG(2k)- | BF1 |
| MAL [50:10:38.5:1.4:0.1] | |
| CICL-1:DSPC:CHOL:DSG-PEG(2k):DSPE-PEG(2k)- | F9 |
| MAL [58:10:30.5:1.4:0.1] | |
| CICL-1:DSPC:CHOL:DMG-PEG(2k):DSPE-PEG(2k)- | F30 |
| MAL [58:10:30.5:1.4:0.1] | |
| CICL-207:DSPC:CHOL:DSG-PEG(2k):DSPE-PEG(2k)- | F50 |
| MAL [58:10:30.5:1.4:0.1] | |
| CICL-207:DSPC:CHOL:DMG-PEG(2k):DSPE-PEG(2k)- | F51 |
| MAL [58:10:30.5:1.4:0.1] | |
| CICL-207:DSPC:CHOL:DMG-PEG(2k):DSPE-PEG(2k)- | F52 |
| MAL [50:10:38.5:1.4:0.1] | |
| CICL-207:DSPC:CHOL:DSG-PEG(2k):DSPE-PEG(2k)- | F53 |
| MAL [62:10:26.5:1.4:0.1] | |
| CICL-215:DSPC:CHOL:DSG-PEG(2k):DSPE-PEG(2k)- | F54 |
| MAL [58:10:30.5:1.4:0.1] | |
| CICL-216:DSPC:CHOL:DSG-PEG(2k):DSPE-PEG(2k)- | F55 |
| MAL [58:10:30.5:1.4:0.1] | |
| CICL-217:DSPC:CHOL:DSG-PEG(2k):DSPE-PEG(2k)- | F56 |
| MAL [58:10:30.5:1.4:0.1] | |
| CICL-218:DSPC:CHOL:DSG-PEG(2k):DSPE-PEG(2k)- | F57 |
| MAL [58:10:30.5:1.4:0.1] | |
| CICL-219:DSPC:CHOL:DSG-PEG(2k):DSPE-PEG(2k)- | F58 |
| MAL [58:10:30.5:1.4:0.1] | |
| CICL-220:DSPC:CHOL:DSG-PEG(2k):DSPE-PEG(2k)- | F59 |
| MAL [58:10:30.5:1.4:0.1] | |
| CICL-221:DSPC:CHOL:DSG-PEG(2k):DSPE-PEG(2k)- | F60 |
| MAL [58:10:30.5:1.4:0.1] | |
| CICL-222:DSPC:CHOL:DSG-PEG(2k):DSPE-PEG(2k)- | F61 |
| MAL [58:10:30.5:1.4:0.1] | |
| CICL-223:DSPC:CHOL:DSG-PEG(2k):DSPE-PEG(2k)- | F62 |
| MAL [58:10:30.5:1.4:0.1] | |
| CICL-224:DSPC:CHOL:DSG-PEG(2k):DSPE-PEG(2k)- | F63 |
| MAL [58:10:30.5:1.4:0.1] | |
| CICL-225:DSPC:CHOL:DSG-PEG(2k):DSPE-PEG(2k)- | F64 |
| MAL [58:10:30.5:1.4:0.1] | |
| CICL-225:DSPC:CHOL:DSG-PEG(2k):DSPE-PEG(2k)- | F65 |
| MAL [62:10:26.5:1.4:0.1] | |
| CICL-225:DSPC:CHOL:DSG-PEG(2k):DSPE-PEG(2k)- | F66 |
| MAL [65:10:23.5:1.4:0.1] | |
| CICL-238:DSPC:CHOL:DSG-PEG(2k):DSPE-PEG(2k)- | F67 |
| MAL [58:10:30.5:1.4:0.1] | |
| CICL-239:DSPC:CHOL:DSG-PEG(2k):DSPE-PEG(2k)- | F68 |
| MAL [58:10:30.5:1.4:0.1] | |

### Example 66. In Vivo Delivery of mRNA by tLNP Incorporating CICL-207 as the Ionizable Cationic Lipid

To assess the ability of a constrained ionizable cationic lipids to facilitate in vivo transfection of T cells with mCherry mRNA (SEQ ID NO. 5), tLNP incorporating CICL-207, or for comparison, CICL-1, and conjugated to an anti-mouse CD5 antibody were prepared and administered to C57BL/6 mice. The tLNP comprised either CICL-207 or CICL-1, DSPC, cholesterol, either DMG-PEG(2000) or DSG-PEG(2000), and DSPE-PEG(2000)-maleimide in the proportions indicated in Table 7 (below) and an N/P ratio of 6. CICL-1 has the structure:

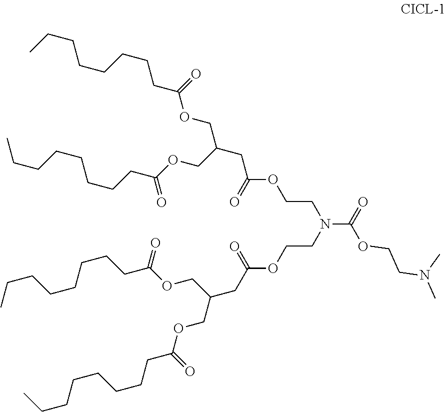 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(C)C]

Briefly, to prepare the tLNP, N1-methylpseudouridine (m1ψ)-substituted mCherry mRNA was encapsulated in LNP using a self-assembly process in which an aqueous solution of mRNA at pH 3.5 was rapidly mixed with a solution of lipids dissolved in ethanol, then followed by stepwise phosphate and Tris buffer dilution and tangential flow filtration (TFF) purification. Then an anti-CD5 mAb was conjugated to the above LNP to generate tLNP. Purified rat anti-mouse CD5 antibody, clone 53-7.3 (BioLegend), was coupled to LNP via N-succinimidyl S-acetylthioacetate (SATA)-maleimide conjugation chemistry. Briefly, LNPs with DSPE-PEG(2000)-maleimide incorporated were formulated and stored at 4° C. on the day of conjugation. The antibody was modified with SATA (Sigma-Aldrich) to introduce sulfhydryl groups at accessible lysine residues allowing conjugation to maleimide. SATA was deprotected using 0.5 M hydroxylamine followed by removal of the unreacted components by G-25 Sephadex Quick Spin Protein columns (Roche Applied Science, Indianapolis, IN). The reactive sulfhydryl group on the antibody was then conjugated to maleimide moieties on the LNPs using thioether conjugation chemistry. Purification was performed using Sepharose CL-4B gel filtration columns (Sigma-Aldrich). tLNPs (LNPs conjugated with a targeting antibody) were frozen at −80° C.

The particle size (hydrodynamic diameter) and polydispersity index of the targeted lipid nanoparticles were determined using dynamic light scattering (DLS) on a Malvern Zetasizer Nano ZS (Malvern Instruments, Worcestershire, UK). Size measurement was carried out in pH 7.4 Tris buffer at 25° C. in disposable capillary cells. A non-invasive back scatter system (NIBS) with a scattering angle of 173° was used for size measurements. mRNA content was determined using a Quant-iT™ RiboGreen RNA assay kit (Invitrogen™). Encapsulation efficiency was calculated by determining the unencapsulated mRNA content by measuring the fluorescence intensity (Fi) upon the addition of RiboGreen® reagent to the LNP and comparing this value to the total fluorescence intensity (Ft) of the RNA content that is obtained upon lysis of the LNPs by 1% Triton X-100, where % encapsulation=(Ft−Fi)/Ft×100). After conjugation, tLNP antibody to mRNA weight ratio (binder density) was determined with the BCA (bicinchoninic acid) total protein assay and Ribogreen® assay of mRNA content.

TABLE 8
Physicochemical properties of the tLNP

| | | | Encapsulation | Ab:mRNA Ratio |
|---|---|---|---|---|
| Composition | Size (nm) | PDI | Efficiency (%) | (wt:wt) by BCA |
| F51 | 91 | 0.09 | 96 | 0.76 |
| F50 | 85 | 0.11 | 96 | 0.71 |
| F52 | 75 | 0.14 | 96 | 0.65 |
| F30 | 81 | 0.07 | 97 | 0.50 |

As seen in Table 8, all of these tLNP compositions had hydrodynamic diameters and polydispersity indices within the acceptable ranges of 50-150 nm and ≤0.2 for PDI. Encapsulation efficiency is acceptable at ≥80% although ≥85% and ≥90% are preferred. Binder density (Ab:mRNA ratio (wt:wt)) is acceptable at ratios of 0.3 to 1.0. Additionally, the stability of these tLNPs at −20° C. was found to be superior to that of tLNPs incorporating a similar lipid lacking a constraining ring.

The apparent or measured pKa of ionizable lipid in the lipid nanoparticle was determined using 6-(p-toluidino)-2-naphthalenesulfonic acid sodium salt (TNS salt, Toronto Research Chemicals, Toronto, ON, Canada). Lipid nanoparticles were diluted in 1×Dulbecco's PBS to a concentration of 1 mM total lipids. TNS salt was prepared as a 1 mg/mL stock solution in DMSO and then further diluted using distilled water to a working solution of 60 μg/mL (179 mM). Diluted lipid nanoparticle samples were further diluted to 90 μM total lipids in 165 μL of buffered solution containing 10 mM HEPES, 10 mM MES, 10 mM ammonium acetate, 130 mM NaCl, and final TNS concentration of 1.33 μg/mL (4 μM) with the pH ranging from 3.5 to 12.2. Following pipette mixing and incubation at room temperature in the dark for 15 min, fluorescence intensity was measured at room temperature in a BioTek Synergy H1 plate reader using excitation and emission wavelengths of 321 and 445 nm, respectively. The fluorescence signal was blank subtracted and plotted as a function of the pH, then analyzed using a nonlinear (Boltzmann) regression analysis with the apparent pKa determined as the pH giving rise to half maximal fluorescence intensity as calculated by the Henderson-Hasselbalch equation.

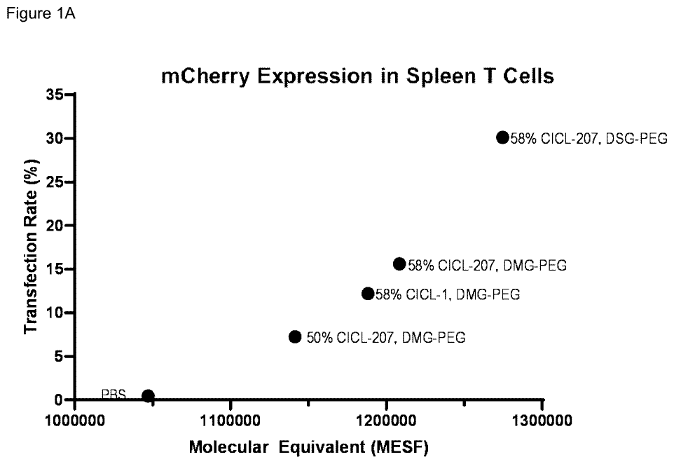

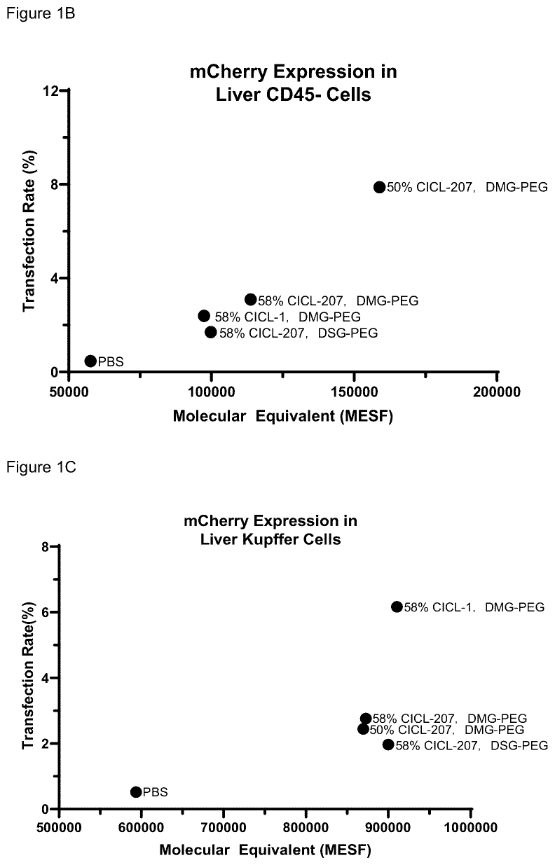

The mice were administered tLNPs containing 10 μg of mRNA by tail vein injection and tissue was harvested 24 hours later. As seen in FIG. 1A, tLNP comprising DMG-PEG(2000) and either CICL-207 or CICL-1 had similar performance in splenic T cells for both transfection rate and expression level (measured as molecular equivalent of soluble fluorochrome (MESF)). Reducing the amount of CICL-207 from 58% to 50% decreased performance, while replacing DMG-PEG(2000) with DSG-PEG(2000) increased performance, with respect to both transfection rate and expression. These same trends have been observed with tLNP comprising CICL-1 as the ionizable lipid (data not shown). The tLNP comprising 58% ionizable cationic lipid had similarly low transfection rates (<4%) for CD45⁻ liver cells (hepatocytes)(FIG. 1B). The tLNP incorporating CICL-207 had similarly low levels in liver Kupffer cells (CD45⁺/CD11⁺ liver cells) although the tLNP incorporating CICL-1 (and DMG-PEG) had a transfection rate that was somewhat higher (˜6%) for Kupffer cells (FIG. 1C).

The best performing composition was the one comprising CICL-207 (58%) and DSG-PEG(2000)—that is high transfection rate and expression level in splenic T cells and low transfection rate and expression level in liver. However, there were no tLNP prepared with CICL-1 and DSG-PEG(2000) for a head-to-head comparison. Therefore, tLNP with 58% ionizable cationic lipid, 10% DSPC, 30.5% cholesterol, 1.4% DSG-PEG(2000), and 0.1% DSPE-PEG(2000)-MAL were prepared as described above. As seen in Table 9, their physicochemical properties were within acceptable ranges.

TABLE 9
Physicochemical properties of the tLNP

| | | | | Ab:mRNA | |
|---|---|---|---|---|---|
| | | | | Ratio | |
| | Size | | Encapsulation | (wt:wt) | Measured pKa |
| Composition | (nm) | PDI | Efficiency (%) | by BCA | (TNS Assay) |
| F9 | 89 | 0.10 | 98 | 0.69 | 6.58 |
| F50 | 82 | 0.05 | 99 | 0.52 | 6.51 |

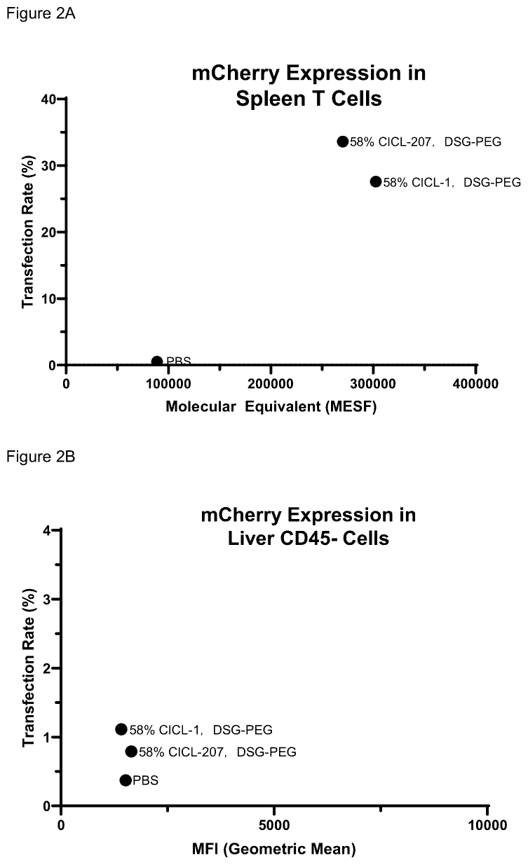

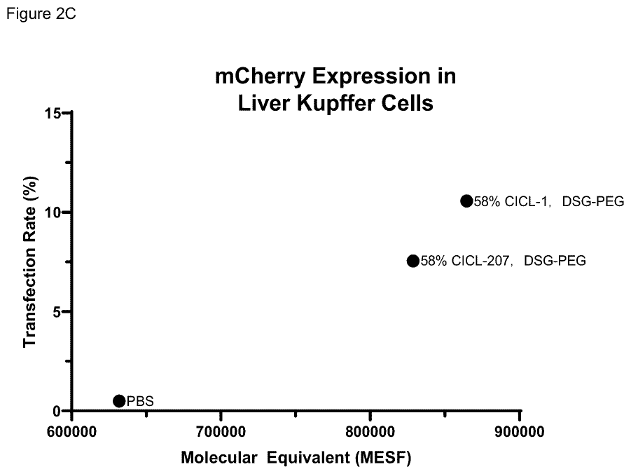

The performance of these tLNP differing only in the ionizable lipid component was similar in all three cell types assessed (FIGS. 2A-2C). In all cases the tLNP comprising CICL-207 had somewhat more desirable transfection rates (higher in splenic T cells, lower in liver cells) and somewhat more desirable expression levels in splenic T cells and CD45⁻ liver cells. Accordingly, tLNP comprising the constrained ionizable cationic lipid CICL-207 as the ionizable cationic lipid performed similarly well to tLNP comprising CICL-1 instead.

### Example 67. In Vivo Delivery of mRNA by LNP Incorporating Further Ionizable Cationic Lipids

Further ionizable cationic lipids according to Formula M2 were incorporated into tLNP and the ability of the tLNP to transfect cells in vivo assessed essentially as described in the preceding Example. Their physicochemical properties as determined in various experiments are compiled in Table 10.

TABLE 10
Physicochemical properties of the tLNP

| | | | | Ab:mRNA | |
|---|---|---|---|---|---|
| | | | | Ratio | |
| | Size | | Encapsulation | (wt:wt) | Measured pKa |
| Composition | (nm) | PDI | Efficiency (%) | by BCA | (TNS Assay) |
| F54 | 88 | 0.02 | 99 | 0.72 | 6.61 |
| F55 | 115 | 0.01 | 99 | 0.78 | 7.05 |
| F56 | 87 | 0.04 | 97 | 0.69 | 5.87 |
| F57 | 92 | 0.04 | 97 | 0.71 | 6.11 |
| F58 | 86 | 0.04 | 98 | 0.55 | 6.20 |
| F59 | 84 | 0.06 | 98 | 0.55 | 6.34 |
| F60 | 99 | 0.07 | 95 | 0.61 | 6.97 |
| F61 | 82 | 0.05 | 98 | 0.58 | 6.40 |
| F62 | 110 | 0.01 | 96 | 0.6 | 6.96 |
| F63 | 112 | 0.03 | 93 | 0.79 | 6.99 |
| F64 | 88 | 0.07 | 93 | 0.70 | 6.47 |
| F67 | 104 | 0.03 | 97 | 0.57 | 7.09 |
| F68 | 87 | 0.03 | 95 | 0.52 | 6.26 |

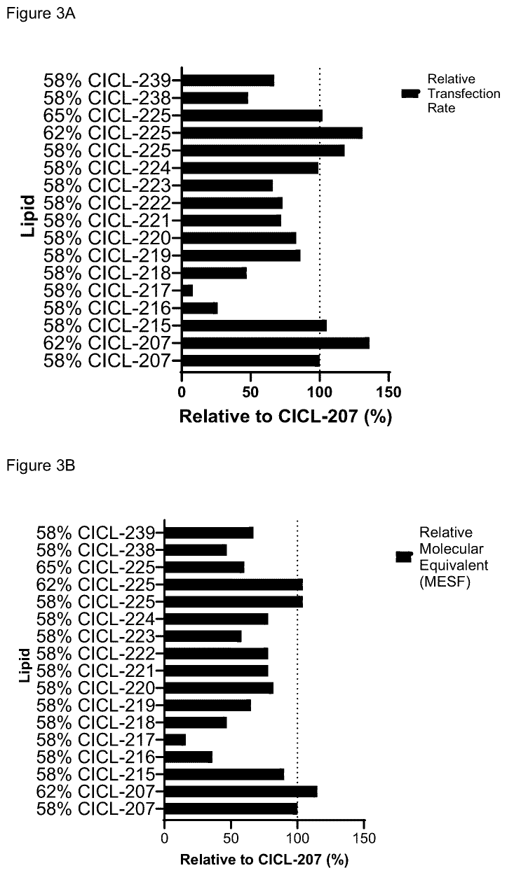

Size, PDI, encapsulation efficiency and antibody:mRNA ratio were all within acceptable limits. Measured pKa for tLNP comprising three of these lipids, CICL-216, -217, and -238, fell outside the range of 6 to 7. Nonetheless, all of these ionizable cationic lipids were able to form tLNP and deliver mRNA into cells. For purposes of comparison, transfection rate and expression level (as mean equivalents of soluble fluorochrome) were normalized to results obtained with tLNP incorporating CICL-207 as the ionizable cationic lipid which was used as a control in each experiment (FIGS. 3A-3B3). The tLNPs comprising CICL-216, -217, or 238 were 3 of the 4 poorest performing tLNP.

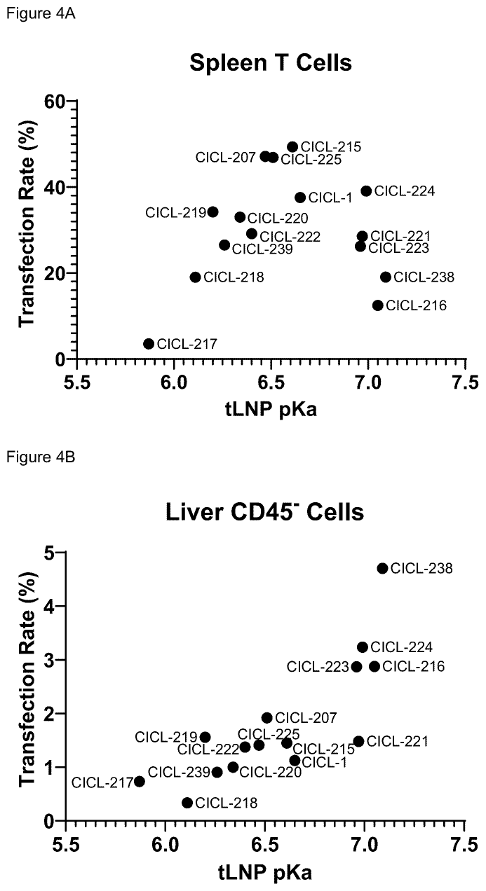

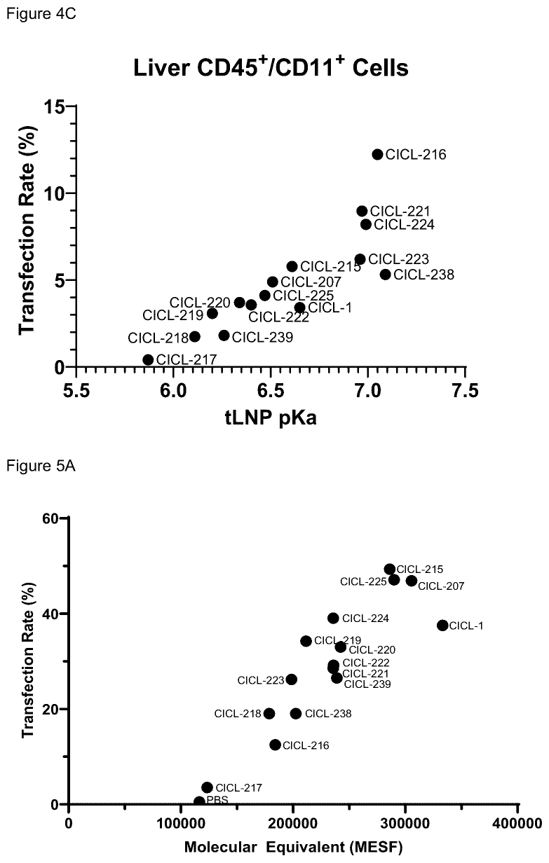

A plot of representative data points for each of these ionizable cationic lipids for transfection rate of splenic T cells versus measured pKa (FIG. 4A) showed increasing transfection rate as pKa approached about 6.6, which then fell as pKa approached and exceeded 7.0. Liver cells, both CD45⁻ (hepatocytes) and CD45⁺/CD11⁺ (Kupffer cells), had generally low transfection rates, under 5% for the CD45⁻ cells and, with the exception of tLNP that incorporated CICL-216, under 10% for the CD45⁺/CD11⁺ cells. Transfection rates for the liver cells trended upward with increasing pKa even as measured pKa exceeded 7.0 (FIGS. 4B-4C).

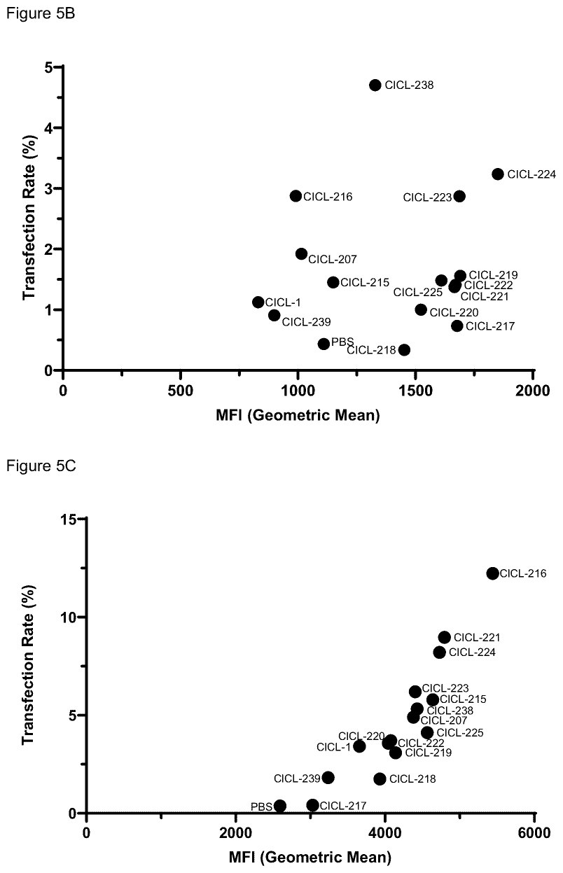

Transfection rate and expression level tended to increase together in splenic T cells and liver CD45⁺/CD11⁺ (Kupffer) cells (FIGS. 5A and 5C, respectively), but there was no clear correlation between transfection rate and expression level in liver CD45⁻ cells (hepatocytes; FIG. 5B).

The data of FIG. 4A indicated that a transfection rate in spleen T cells of ca. 50% was realized at a measured pKa in tLNP formulation of ca. pKa=6.6. At more basic measured pKa, as in the case of CICL-224 (measured pKa ca. 7.0), the transfection rate fell below 40%. At less basic measured pKa, as in the case of CICL-218 (measured pKa ca. 6.20), the transfection rate fell to ca. 20%. Alterations in the basic head group were designed to lower or increase calculated basicity of some of the ionizable cationic lipids, with attendant changes in measured pKa in tLNPs to move the lipid toward a more preferred measured pKa of ca. 6.6. Table 11 presents modifications to CICL-224 and CICL-218 which modulated calculated pKa as well as measured pKa for some of these lipids. CICL-224 has a calculated pKa (c-pKa) of 8.42 and a measured pKa (in tLNP formulation) of 6.98. Changing the basic head group to N-methylpiperazine (CICL-239) lowered the calculated pKa to 7.16 and measured pKa to 6.25. The measured pKa values for CICL-224 and CICL-239 bracketed the pKa=ca. 6.60 value of the lipids with the highest transfection rates shown in FIG. 4A. Changing the basic head group to N-ethylpiperazine for CICL-305 resulted in a c-pKa of 7.65, between of the c-pKa's of CICL-224 and CICL-239 which should move measured pKa toward the 6.6 value associated with higher transfection rates. The measured pKa for CICL-218 (c-pKa=8.49, pKa=6.07) is lower than associated with optimal performance. CICL-306 and CICL-307 were designed to raise pKa by added a CH₂ group between the ester O and basic N if the dimethylamine group or by altering the basic dimethylamine group to a diethylamine and indeed these lipids have c-pKa values of 9.45 and 9.27, respectively. All of the lipids in Table 11 exhibit c Log D values in an acceptable range of 13-15. As c Log D is increased for any set value of c-pKa the measured pKa of the tLNP will tend to decrease.

TABLE 11
Modifications of CICL-224 and CICL-218 Modulating pKa

| | | | | Measured |
|---|---|---|---|---|
| Structure | | cLogD | c-pKa | pKa |
| 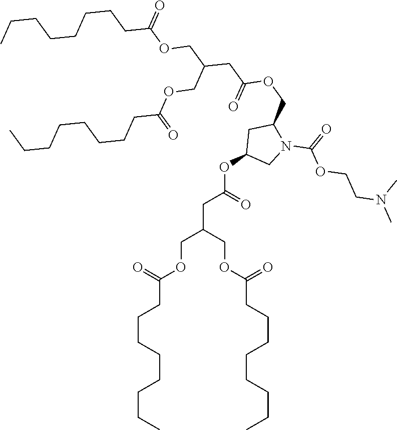 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OCCN(C)C] | CICL-224 | 13.75 | 8.42 | 6.98 |
| 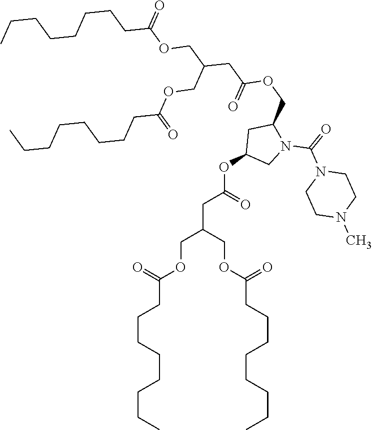 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)N1CCN(C)CC1] | CICL-239 | 14.77 | 7.16 | 6.25 |
| 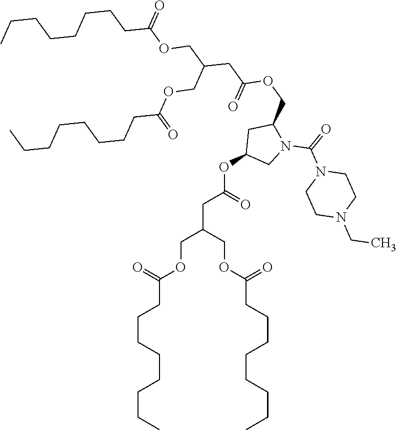 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)N1CCN(CC)CC1] | CICL-304 | 14.79 | 7.65 | NT |
| 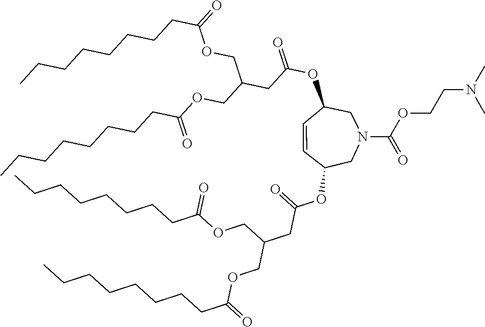 | CICL-218 | 13.71 | 8.49 | 6.07 |
| 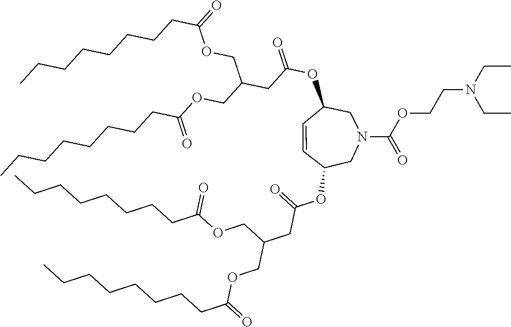 | CICL-305 | 13.84 | 9.45 | NT |
| 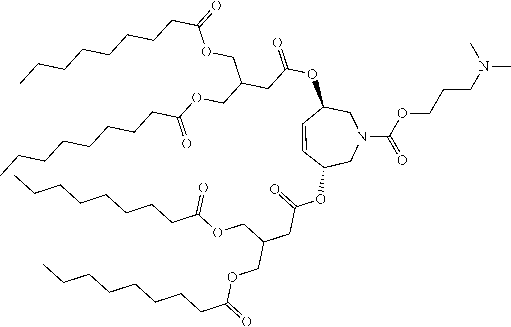 | CICL-306 | 13.18 | 9.27 | NT |

Further modifications of CICL-224 and CICL-218 were designed by altering their basic head groups to lower and raise pKa, respectively, toward a measured pKa=ca. 6.6 and c-pKa calculated as shown in Table 12.

TABLE 12
Further modifications of CICL-224 and CICL-218 to Modulate pKa

| Structure | B | c-pKa |
|---|---|---|
| 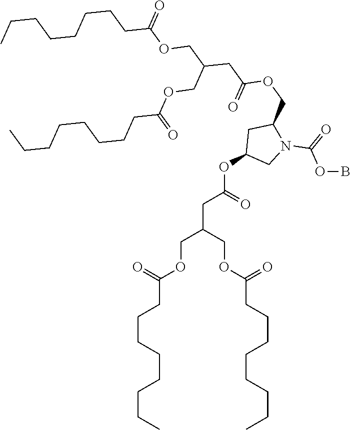 | 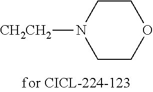 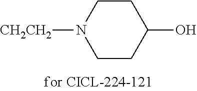 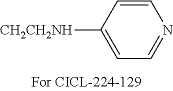 | 6.17 7.77 7.90 |
| 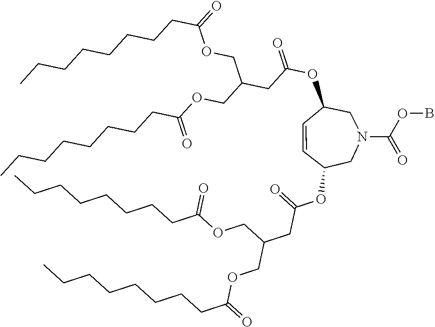 | 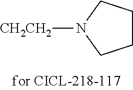 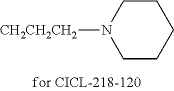 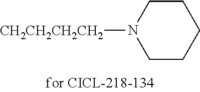 | 9.42 9.24 9.61 |

### Example 68. Evaluation of tLNP with Increased Ionizable Cationic Upid Content

As seen in Example 60 (above), increasing the content of ionizable cationic lipid in tLNPs tended to increase transfection rate and expression level. To further assess this trend tLNP containing 62% CICL-207 or CICL 225, or 65% CICL-225 were made. Their physicochemical properties as determined in various experiments are compiled in Table 13.

TABLE 13
Physicochemical properties of the tLNP

| | | | | Ab:mRNA | |
|---|---|---|---|---|---|
| | | | | Ratio | Measured |
| | Size | | Encapsulation | (wt:wt) | pKa |
| Composition | (nm) | PDI | Efficiency (%) | by BCA | (TNS Assay) |
| F50 | 81.87 | 0.05 | 98.73 | 0.52 | 6.51 |
| F53 | 95.19 | 0.06 | 97.58 | 0.64 | 6.36 |
| F64 | 88 | 0.07 | 93 | 0.7 | 6.47 |
| F65 | 95.11 | 0.02 | 96.24 | 0.65 | 6.56 |
| F66 | 105 | 0.00 | 85.13 | 0.66 | 6.40 |

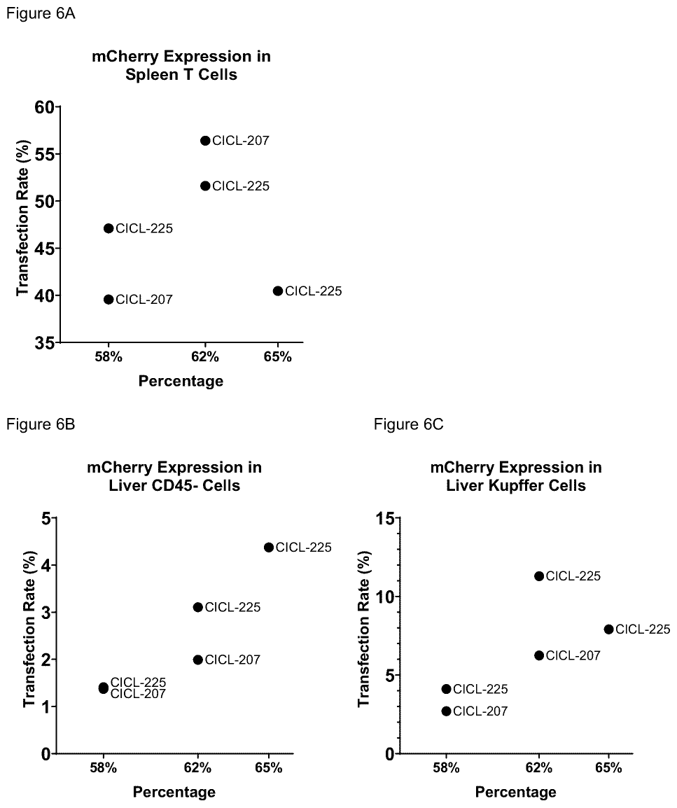

Increasing the ionizable cationic lipid content of the tLNPs to 62% increased transfection rate in all three cell types tested (FIGS. 6A-6C). Increasing the ionizable cationic lipid content of the tLNPs further to 65% decreased transfection rate in splenic T cells and liver Kupffer cells indicating that the optimal content of ionizable cationic lipid for this LNP composition had been exceeded. In contrast, increasing the ionizable cationic lipid content of the tLNPs further to 65% further increased transfection rate for CD45⁻ liver cells (hepatocytes), though it remained less than 5%. In this data set, the increase in transfection rate associated with raising ionizable cationic lipid content from 58% to 62% was greater for CICL-207 than for CICL-225 in splenic T cells while the increased transfection rate for CICL-225 was greater in both types of liver cells. In some embodiments, 58% ionizable cationic lipid can be preferred over 62% in order to minimize transfection of liver cells.

### Example 69: Assessment of F9 tLNP and F50 tLNP in NCG Mice

LNPs with the F9 and F50 composition were prepared as described in Example 66, except tLNPs were generated by conjugating a humanized anti-CD8 antibody through use of an AJICAP reagent. Specifically, an antibody with the CBD1033 heavy and light chains (SEQ ID NOs: 1 and 2, respectively). The tLNP encapsulated N1-methylpseudouridine-substituted mRNA encoding an anti-CD19 CAR (SEQ ID NO: 3).

Female NCG mice (˜10 weeks old, Charles River Laboratories) were injected intravenously via the tail vein with 10 million human PBMCs. After 14 days of T cell engraftment mice were injected intravenously with the indicated tLNPs. 6 hours after injection, mice were sacrificed, and blood and spleen were analyzed for CAR expression and B cell depletion by flow cytometry.

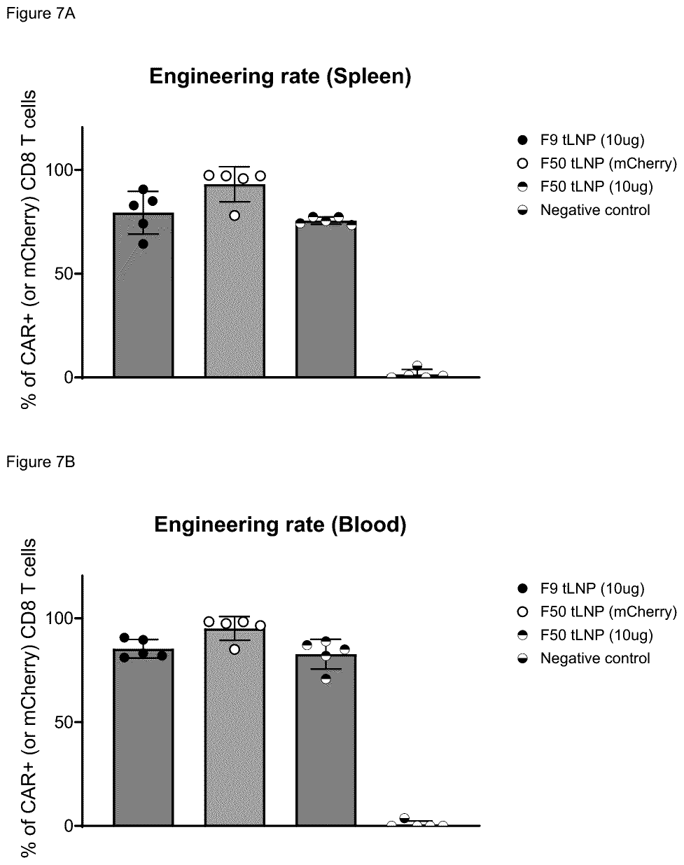

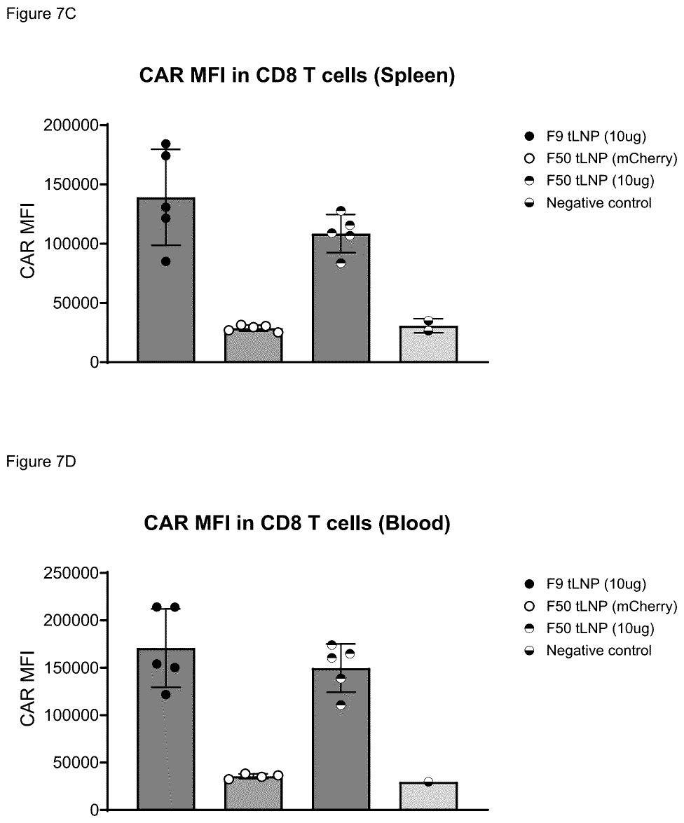

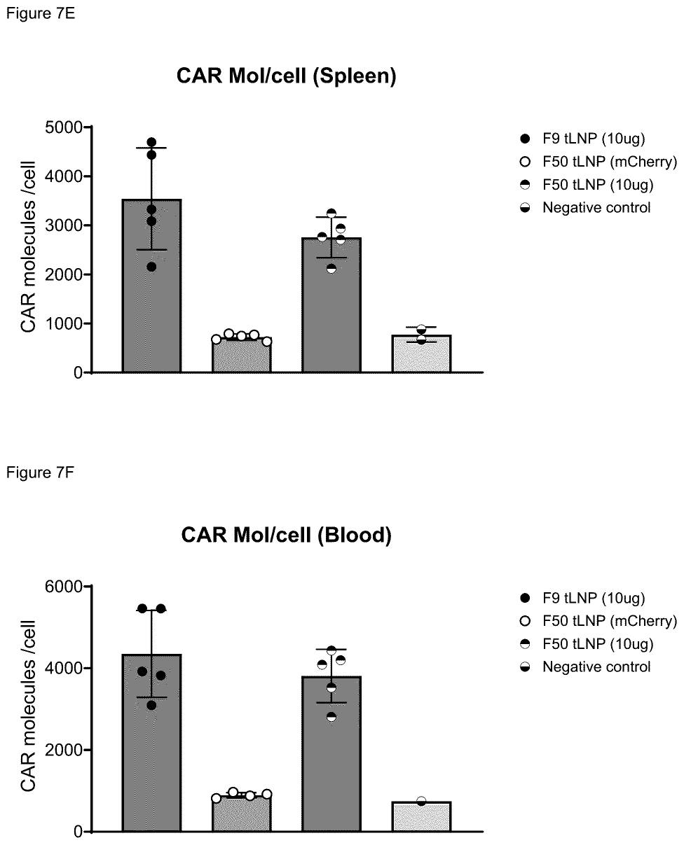

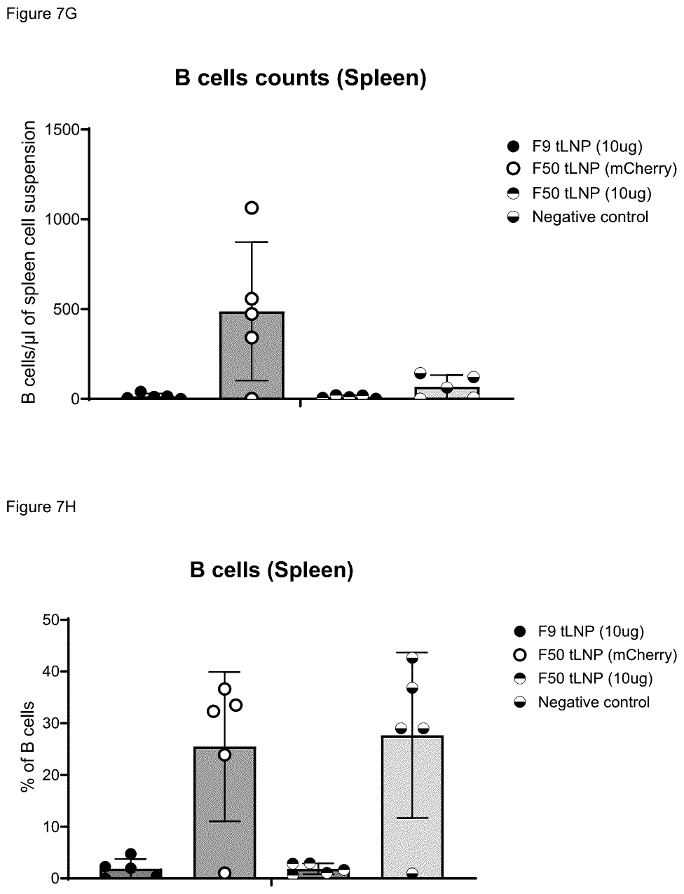

The F9 and F50 tLNPs decorated with the anti-CD8 antibodies achieved similar transfection rates in CD8⁺ blood and spleen T cells. Both tLNPs showed comparable CAR engineering rates (FIG. 7A-B) and CAR expression (as MFI; FIG. 7C-D) in CD8⁺ blood and spleen T cells, as well as the comparable CAR molecules/cell (FIG. 7E-F). Both tLNPs also induced complete B cell depletion 6-hours after administration (FIG. 7G-H).

### Example 70: Assessment of BF1 LNP, BF1 tLNP, F50 LNP, F50 tLNP by Luciferase Biodistribution

LNP and tLNP with the F9 and F50 composition were prepared as example 66 described, except the payload consisted of mRNA encoding luciferase (SEQ ID NO. 4).

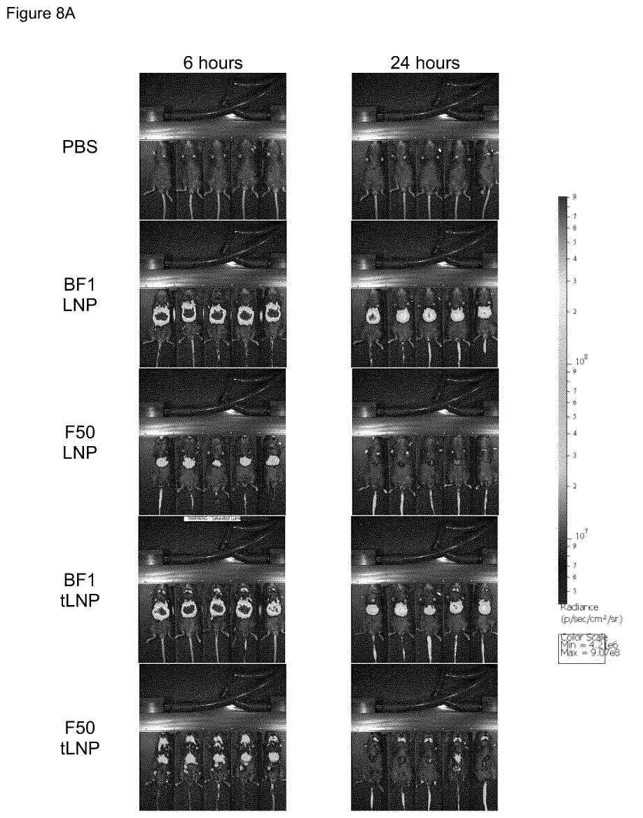

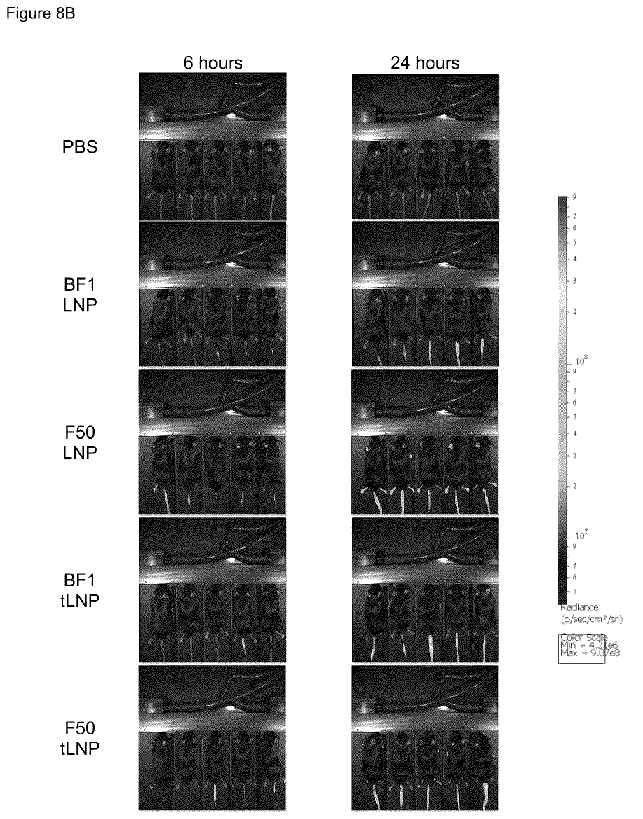

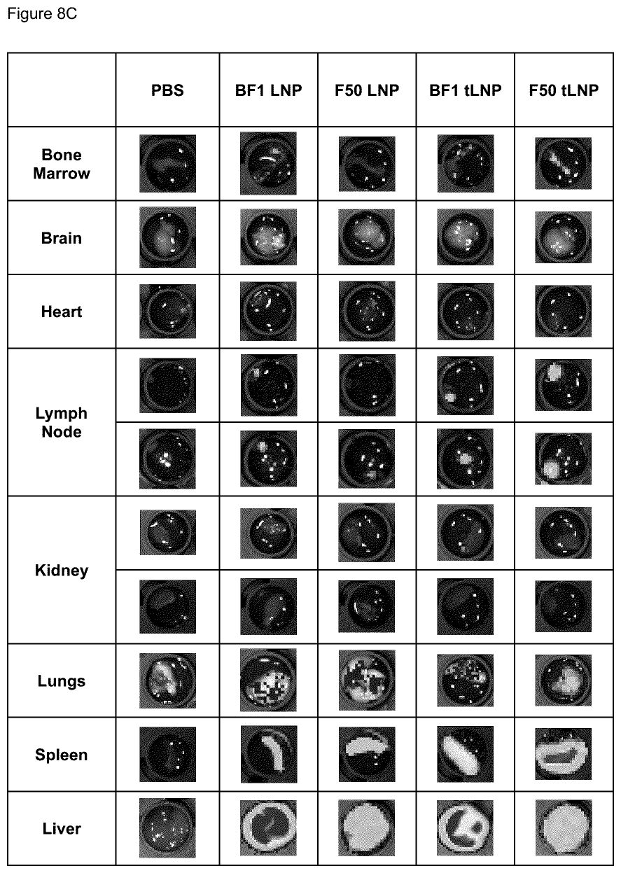

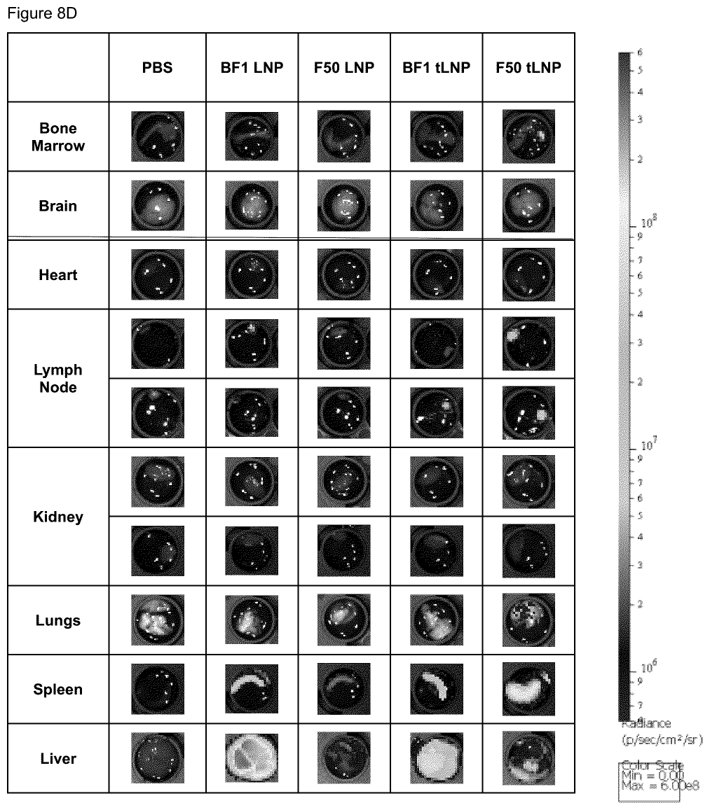

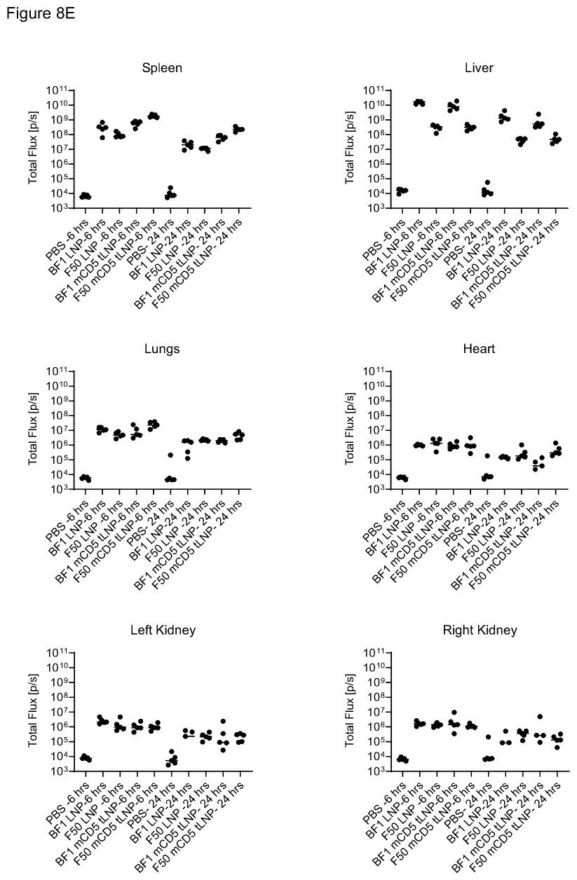

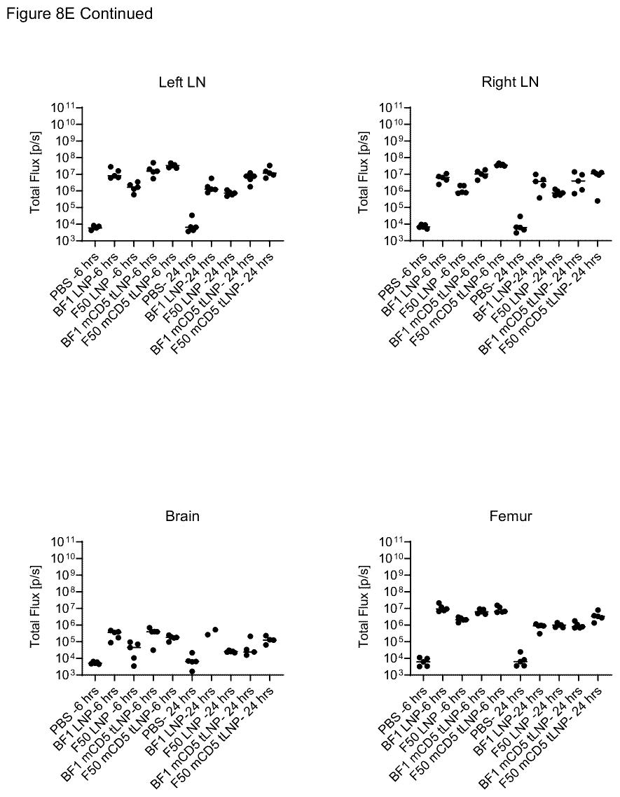

Eight-week-old female C57BI)6 mice (Charles River Laboratories) were intravenously injected with BF1 or F50 LNPs or CD5-targeted tLNPs encapsulating mRNA encoding luciferase at a dose of 2 μg mRNA/animal via the tail vein. At 6 hours post-injection, prone and supine bioluminescence images were collected from all mice (FIGS. 8A-8B). Following whole-animal imaging, the mice were sacrificed, and the following tissues were collected: liver, spleen, lung, kidneys (both), heart, bone marrow (femur), lymph nodes (left and right inguinal), and brain. Tissues were placed into luciferin pre-filled black polystyrene plates with spacing between tissues, and bioluminescent images were collected at 6 hours and 24 hours post-injection (FIGS. 8C-8D, respectively). Quantitative results (FIG. 8E) show that use of CICL-207 reduced bioluminescence in the liver about 2 logs compared to the BF1 compositions yet for the tLNP incorporating CICL-207 bioluminescence in the spleen was similar or greater than the BF1 compositions and about 1 log greater than in the liver.

These compositions displayed the general pattern that bioluminescence in spleen>lymph node>lung>bone marrow>kidney>heart>brain, with the CICL-207 containing tLNP composition generally producing the least bioluminescence in each of the non-target organs.

### Example 71: Delivery of CRISPR Gene-Editing Components In Vivo

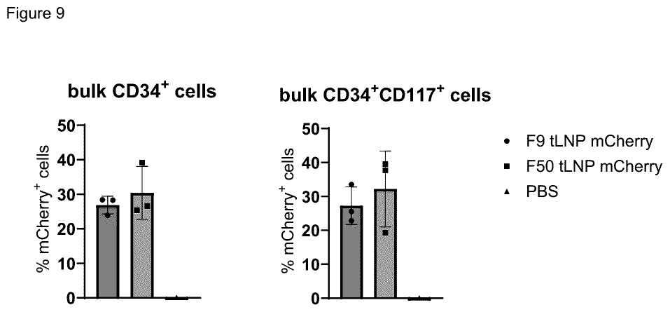

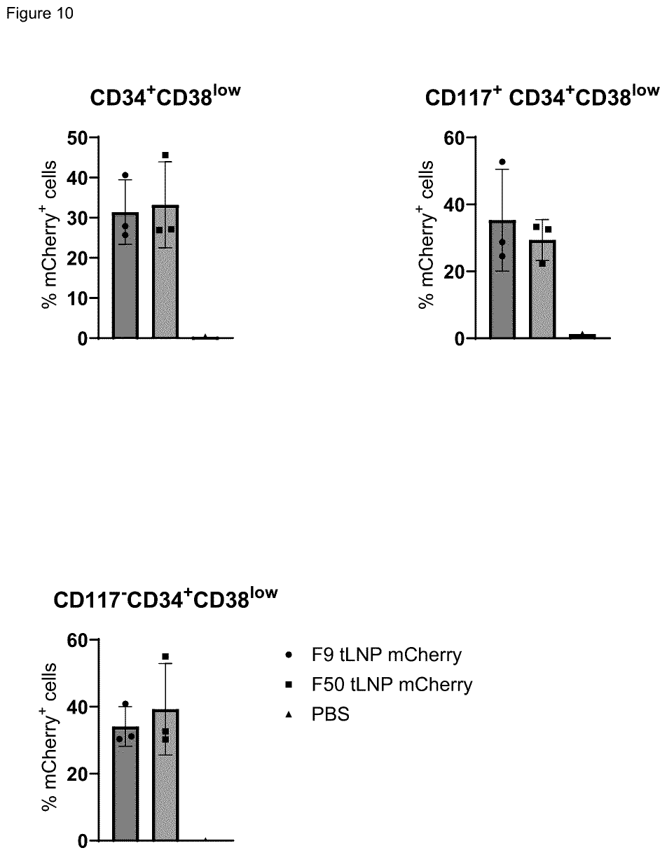

To assess the ability of the disclosed tLNP to deliver gene-editing components to human hematopoietic stem cells (CD34+ CD117+) in vivo, female NCG mice engrafted with human CD34+ cells (˜25-29 weeks after engraftment, Charles River Laboratories) were intravenously administered CD117-targeted tLNPs encapsulating mRNA payload encoding the mCherry reporter protein (SEQ ID NO. 5). Both F9 and F50 tLNP were able to deliver mCherry payload to all analyzed subsets of CD34+ cells (FIGS. 9-10).

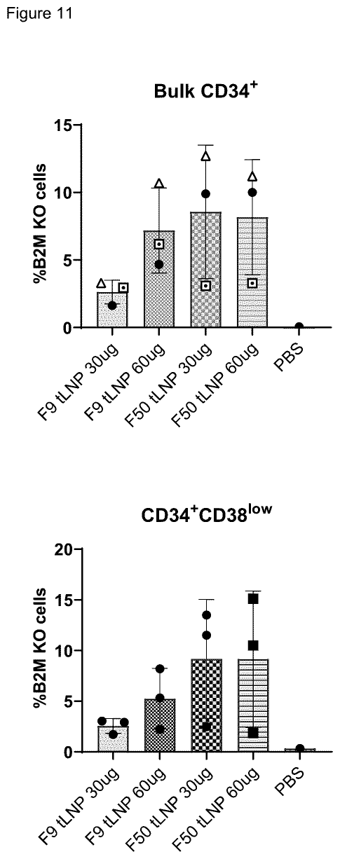

Female NCG mice engrafted with human CD34+ cells (˜25-29 weeks after engraftment Charles River Laboratories) were intravenously administered CD117-targeted tLNPs. The tLNP used the F50 lipid composition with and overall N/P ratio of 6 and a 1:1 (w/w) SpCas9:sgRNA payload. sgRNA targeted the B2M locus and successful gene editing was measured as loss of B2M expression. Eight days after infusion, bone marrow was collected and analyzed by flow cytometry for B2M knockout on indicated populations of CD34+ cells. The results are presented in FIG. 11 and demonstrate effective delivery of SpCas9 and the sgRNA into CD34+ cells in vivo. Editing of bone marrow CD34⁺ or CD34⁺CD38^{low} cells in the humanized CD34⁺ NCG mice by F50 tLNP was approximately 10%, for both 30 ug and 60 ug dose, with a single dose of CD117-targeted tLNPs.

### Example 72: Tolerability of F50 LNP

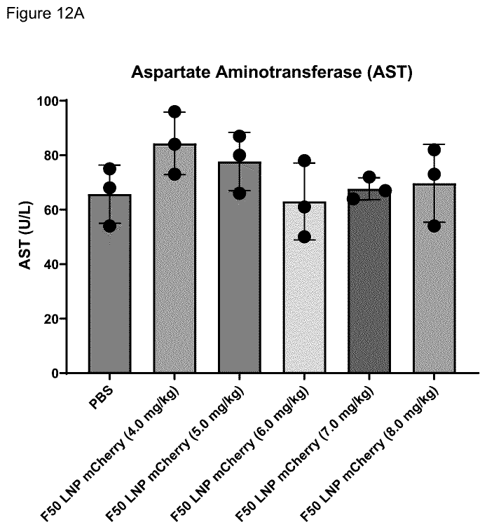

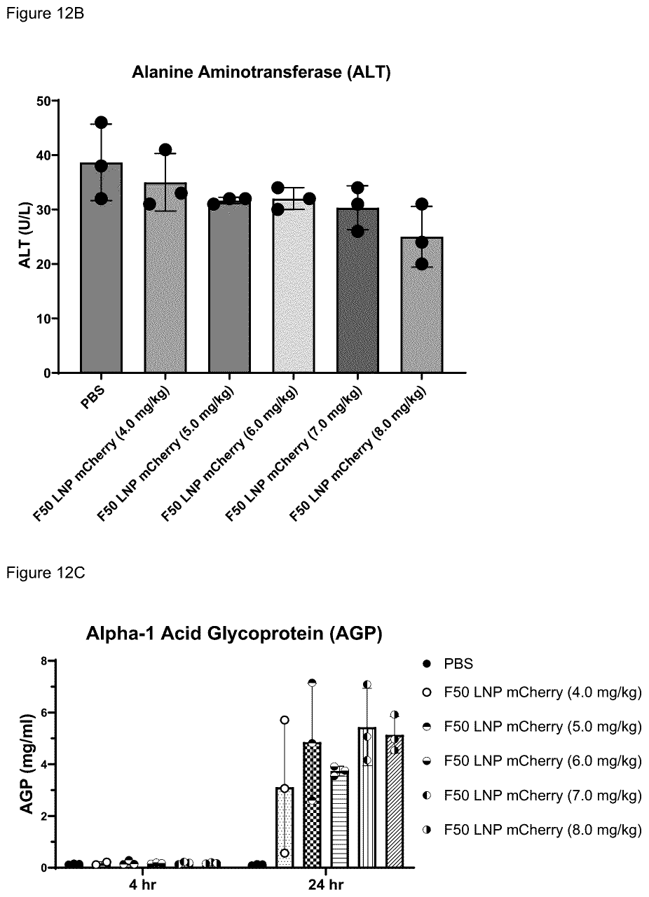

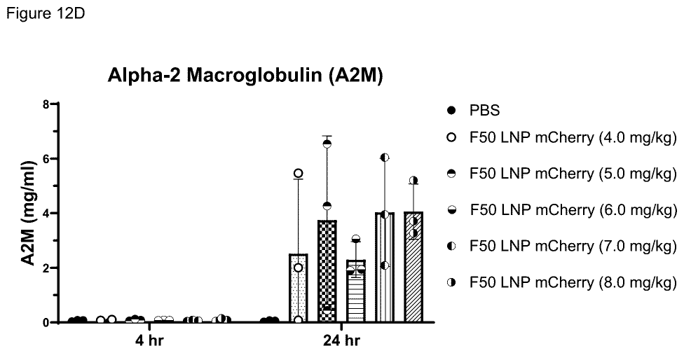

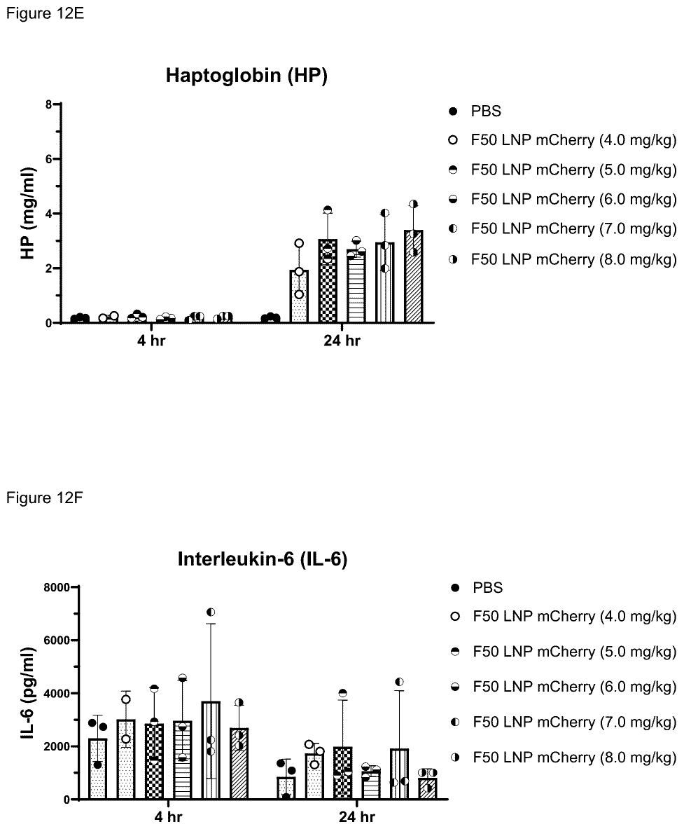

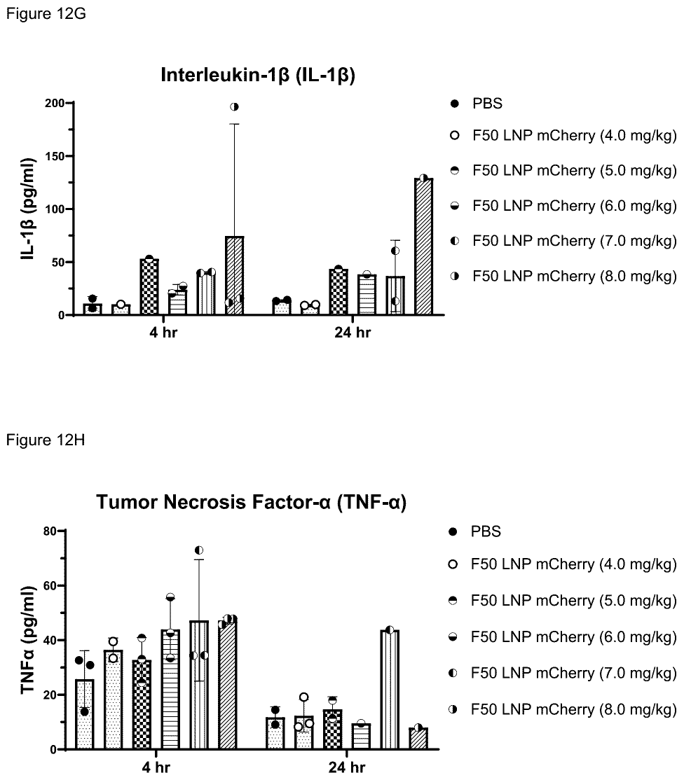

A study in which various dosages of undecorated F50 LNP encapsulating mCherry mRNA (up to 8 mg/kg) were administered intravenously to 8-week-old male Sprague Dawley rats (Charles River Laboratories). Liver enzymes in serum were determined (FIGS. 12A-B). Acute phase protein (FIGS. 12C-E) and cytokine levels (FIGS. 12F-H) were assessed in plasma by Luminex assay. There were only minimal increases in liver enzyme levels. The acute phase proteins were mildly elevated at 24 hours. The cytokine elevations were transient and resolved by 24 hours. None of these led to any clinical observations. There is alack of dose responsiveness in the elevations of acute phase proteins and cytokines suggesting that it is a general response to LNP infusion and does not indicate a lack of tolerability of the LNP components. These data demonstrate that even at these high doses, LNPs incorporating CICL-207 were well tolerated. Prior experience also indicates that addition of an antibody to an LNP, especially one that targets a non-liver tissue as in a tLNP, leads to reduction in any elevation of liver enzymes.

Additional aspects of the disclosure are provided by the following enumerated embodiments, which can be combined in any number and in any combination not technically or logically inconsistent. This is not an exhaustive listing of the embodiments disclosed which include similar embodiments directed to different species and genera than those exemplified here. Furthermore, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and enumerated below.

Embodiment 1. An ionizable cationic lipid having a structure of formula M2,

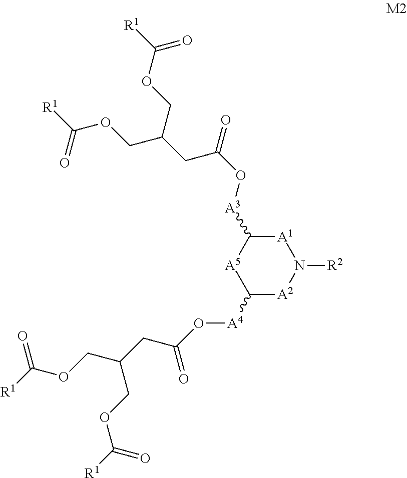

wherein X is

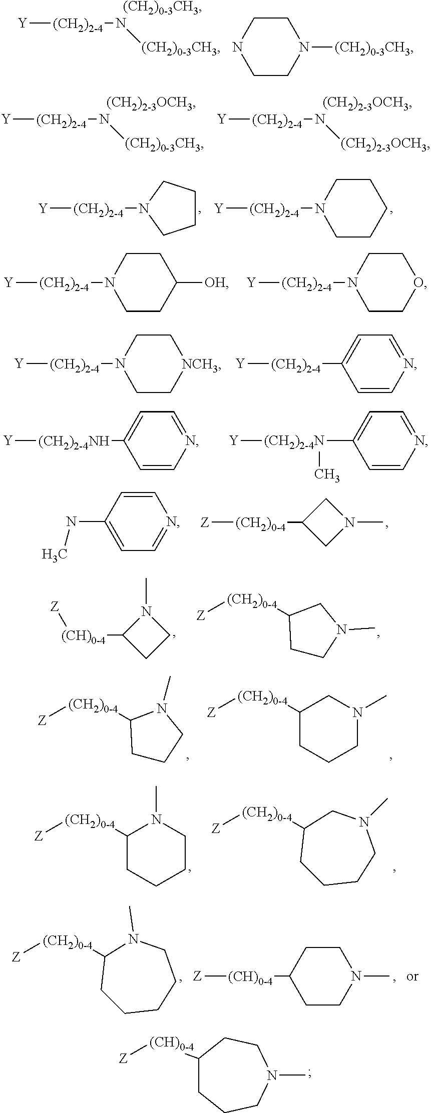

Y is O, S, NH, or NCH₃;
Z is O, NH, or NCH₃;
each R¹ is independently selected from C₇-C₁₁ alkyl or C₇-C₁₁ alkenyl; and
A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂; or
A¹ is (CH₂)₀, A² is (CH₂)₁, A³ is (CH₂)₁, A⁴ is (CH₂)₀, and A⁵ is (CH₂)₁; or
A¹ is (CH₂)₁, A² is (CH₂)₁, A³ is (CH₂)₀, A⁴ is (CH₂)₀, and A⁵ is (CH₂)₀; or
A¹ is (CH₂)₁, A² is (CH₂)₁, A³ is (CH₂)₀, A⁴ is (CH₂)₀, and A⁵ is (CH₂)₁; or
A¹ is (CH₂)₁, A² is (CH₂)₁, A³ is (CH₂)₀, A⁴ is (CH₂)₀, and A⁵ is (CH₂)₂ or CH═CH, and
wherein the wavy bonds indicate that any relative or absolute stereo-configuration of the corresponding ring atom, or mixtures of stereo-configurations, can be assumed.

Embodiment 2. The ionizable cationic lipid of embodiment 1 wherein A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

Embodiment 3. The ionizable cationic lipid of embodiment 2, wherein A¹ is (CH₂)₀, A² is (CH₂)₀, A³ is (CH₂)₁, A⁴ is (CH₂)₁, and A⁵ is (CH₂)₁.

Embodiment 4. The ionizable cationic lipid of embodiment 2, wherein A¹ is (CH₂)₀, A² is (CH₂)₀, A³ is (CH₂)₁, A⁴ is (CH₂)₁, and A⁵ is (CH₂)₂.

Embodiment 5. The ionizable cationic lipid of embodiment 2, wherein A¹ is (CH₂)₀, A² is (CH₂)₀, A³ is (CH₂)₁, A⁴ is (CH₂)₁, and A⁵ is (CH₂)₃.

Embodiment 6. The ionizable cationic lipid of embodiment 2, wherein A¹ is (CH₂)₀, A² is (CH₂)₀, A³ is (CH₂)₁, A⁴ is (CH₂)₁, and A⁵ is (CH₂)₄.

Embodiment 7. The ionizable cationic lipid of embodiment 2, wherein A¹ is (CH₂)₀, A² is (CH₂)₀, A³ is (CH₂)₁, A⁴ is (CH₂)₁, and A⁵ is CH₂—CH═CH—CH₂ Embodiment 8. The ionizable cationic lipid of embodiment 1 wherein A¹ is (CH₂)₀, A² is (CH₂)₁, A³ is (CH₂)₁, A⁴ is (CH₂)₀, and A⁵ is (CH₂)₁.

Embodiment 9. The ionizable cationic lipid of embodiment 1 wherein A¹ is (CH₂)₁, A² is (CH₂)₁, A³ is (CH₂)₀, A⁴ is (CH₂)₀, and A⁵ is (CH₂)₀.

Embodiment 10. The ionizable cationic lipid of embodiment 1 wherein A¹ is (CH₂)₁, A² is (CH₂)₁, A³ is (CH₂)₀, A⁴ is (CH₂)₀, and A⁵ is (CH₂)₁.

Embodiment 11. The ionizable cationic lipid of embodiment 1 wherein A¹ is (CH₂)₁, A² is (CH₂)₁, A³ is (CH₂)₀, A⁴ is (CH₂)₀, and A⁵ is (CH₂)₂ or CH═CH.

Embodiment 12. The ionizable cationic lipid of embodiment 11, wherein A¹ is (CH₂)₁, A² is (CH₂)₁, A³ is (CH₂)₀, A⁴ is (CH₂)₀, and A⁵ is (CH₂)₂.

Embodiment 13. The ionizable cationic lipid of embodiment 11, wherein A¹ is (CH₂)₁, A² is (CH₂)₁, A³ is (CH₂)₀, A⁴ is (CH₂)₀, and A⁵ is CH═CH.

Embodiment 14. The ionizable cationic lipid of any one of embodiments 1 to 13, wherein each R¹ is independently selected from C₇-C₁₀ alkyl or C₇-C₉ alkyl.

Embodiment 15. The ionizable cationic lipid of any one of embodiments 1 to 13, wherein each R¹ is independently selected from a linear C₇-C₁₁ alkyl, e.g., a linear C₇-C₁₀ alkyl, or a linear C₇-C₉ alkyl.

Embodiment 16. The ionizable cationic lipid of any one of embodiments 1 to 13, wherein each R¹ is independently selected from (CH₂)₆₋₈CH₃.

Embodiment 17. The ionizable cationic lipid of any one of embodiments 1 to 13, wherein R¹ is (CH₂)₇CH₃.

Embodiment 18. The ionizable cationic lipid of any one of embodiments 1 to 13, wherein each R¹ is independently selected from a linear C₇-C₁₁ alkenyl, e.g., a linear C₇-C₁₀ alkenyl, or a linear C₇-C₉ alkenyl.

Embodiment 19. The ionizable cationic lipid of any one of embodiments 1 to 13, wherein each R¹ is a linear C₈ alkenyl.

Embodiment 20. The ionizable cationic lipid of any one of embodiments 1 to 13, wherein each R¹ is independently selected from a branched C₇-C₁₁ alkyl, e.g., C₇-C₁₀ alkyl, or C₇-C₉ alkyl.

Embodiment 21. The ionizable cationic lipid of any one of embodiments 1 to 13, wherein each R¹ is a branched C₈ alkyl.

Embodiment 22. The ionizable cationic lipid of any one of embodiments 1 to 13, wherein each R¹ is independently selected from a branched C₇-C₁₁ alkenyl, e.g., C₇-C₁₁ alkenyl, or C₇-C₉ alkenyl.

Embodiment 23. The ionizable cationic lipid of any one of embodiments 1 to 13, wherein each R¹ is a branched C₈ alkenyl.

Embodiment 24. The ionizable cationic lipid of any one of embodiments 1 to 13, wherein R¹ is a branched alkyl or alkenyl, the branch point is positioned so that ester carbonyls are not in an a position relative to the branch point, for example they are in a β position relative to the branch point.

Embodiment 25. The ionizable cationic lipid of any one of embodiments 1 to 24, wherein each R¹ is the same.

Embodiment 26. The ionizable cationic lipid of any one of embodiments 1 to 25 having the structure:

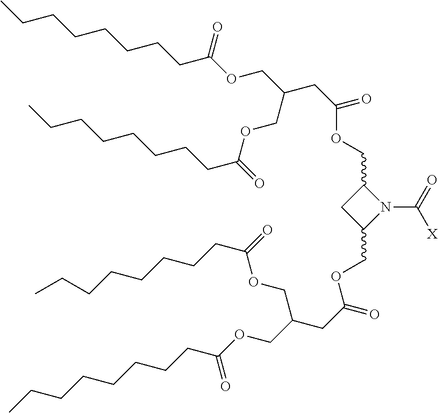

wherein wavy bonds indicate that any relative or absolute stereo-configuration of the corresponding ring atom can be assumed.

Embodiment 27. The ionizable cationic lipid of any one of embodiment 1 to 25 having the structure:

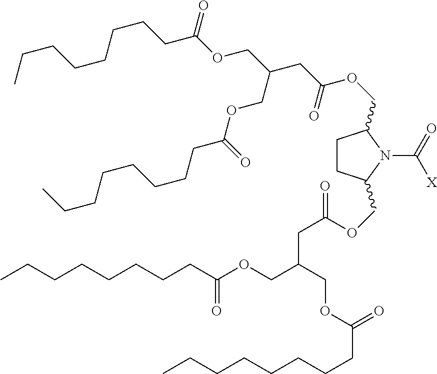

wherein wavy bonds indicate that any relative or absolute stereo-configuration of the corresponding ring atom, or mixtures of stereo-configurations, can be assumed.

Embodiment 28. The ionizable cationic lipid of any one of embodiments 1 to 25 having the structure:

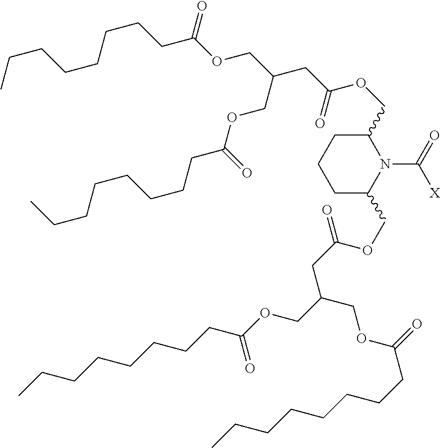

wherein wavy bonds indicate that any relative or absolute stereo-configuration of the corresponding ring atom, or mixtures of stereo-configurations, can be assumed.

Embodiment 29. The ionizable cationic lipid of any one of embodiments 1 to 25 having the structure:

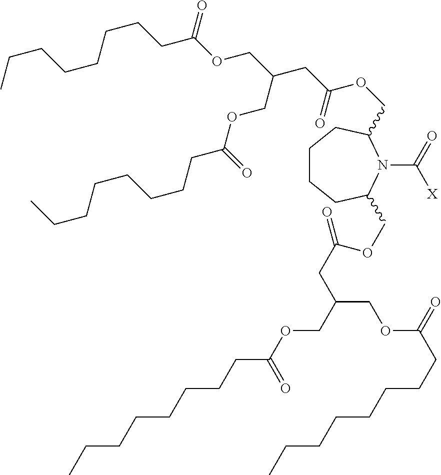

wherein wavy bonds indicate that any relative or absolute stereo-configuration of the corresponding ring atom, or mixtures of stereo-configurations, can be assumed.

Embodiment 30. The ionizable cationic lipid of any one of embodiments 1 to 25 having the structure:

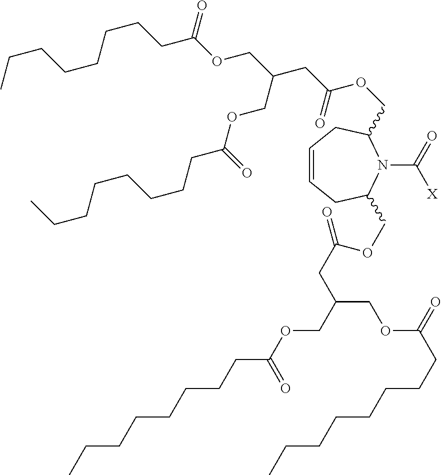

wherein wavy bonds indicate that any relative or absolute stereo-configuration of the corresponding ring atom, or mixtures of stereo-configurations, can be assumed.

Embodiment 31. The ionizable cationic lipid of any one of embodiments 1 to 25 having the structure:

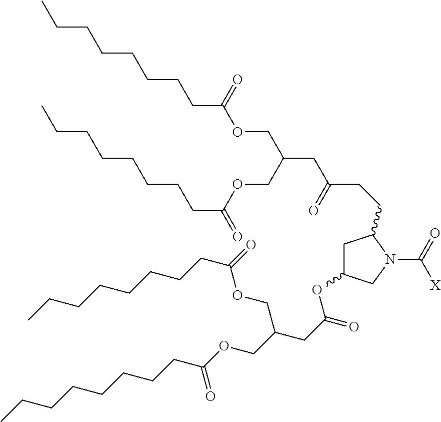

wherein wavy bonds indicate that any relative or absolute stereo-configuration of the corresponding ring atom, or mixtures of stereo-configurations, can be assumed.

Embodiment 32. The ionizable cationic lipid of any one of embodiments 1 to 25 having the structure:

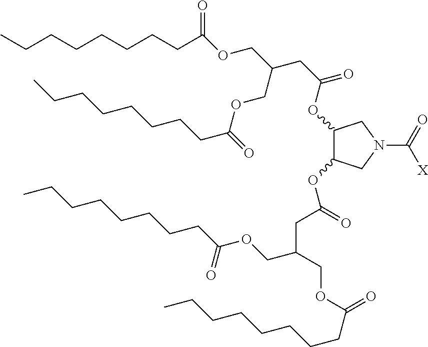

wherein wavy bonds indicate that any relative or absolute stereo-configuration of the corresponding ring atom, or mixtures of stereo-configurations, can be assumed.

Embodiment 33. The ionizable cationic lipid of any one of embodiments 1 to 25 having the structure:

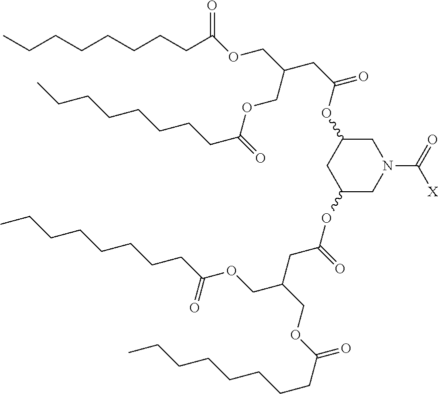

wherein wavy bonds indicate that any relative or absolute stereo-configuration of the corresponding ring atom, or mixtures of stereo-configurations, can be assumed.

Embodiment 34. The ionizable cationic lipid of any one of embodiments 1 to 25 having the structure:

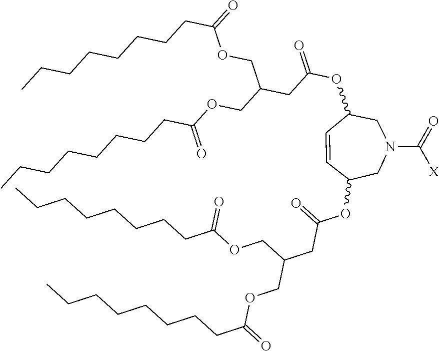

wherein wavy bonds indicate that any relative or absolute stereo-configuration of the corresponding ring atom, or mixtures of stereo-configurations, can be assumed.

Embodiment 35. The ionizable cationic lipid of any one of embodiments 1 to 25 having the structure:

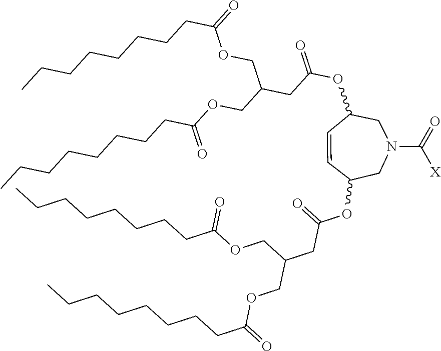

wherein wavy bonds indicate that any relative or absolute stereo-configuration of the corresponding ring atom, or mixtures of stereo-configurations, can be assumed.

Embodiment 36. The ionizable cationic lipid of any one of embodiments 1 to 35 wherein X is

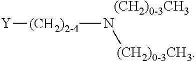

Embodiment 37. The ionizable cationic lipid of any one of embodiments 1 to 35 wherein X is

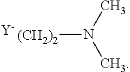

Embodiment 38. The ionizable cationic lipid of any one of embodiments 1 to 35, wherein X is

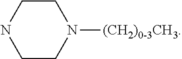

Embodiment 39. The ionizable cationic lipid of any one of embodiments 1 to 35, wherein X is

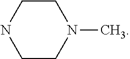

Embodiment 40. The ionizable cationic lipid of any one of embodiments 1 to 35, wherein X is

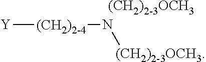

Embodiment 41. The ionizable cationic lipid of any one of embodiments 1 to 35, wherein X is

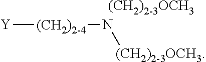

Embodiment 42. The ionizable cationic lipid of any one of embodiments 1 to 35, wherein X is

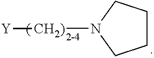

Embodiment 43. The ionizable cationic lipid of any one of embodiments 1 to 35, wherein X is

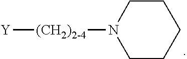

Embodiment 44. The ionizable cationic lipid of any one of embodiments 1 to 35, wherein X is

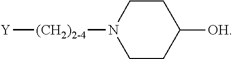

Embodiment 45. The ionizable cationic lipid of any one of embodiments 1 to 35, wherein X is

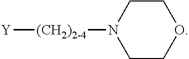

Embodiment 46. The ionizable cationic lipid of any one of embodiments 1 to 35, wherein X is

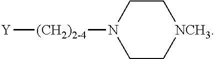

Embodiment 47. The ionizable cationic lipid of any one of embodiments 1 to 35, wherein X is

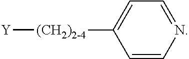

Embodiment 48. The ionizable cationic lipid of any one of embodiments 1 to 35, wherein X is

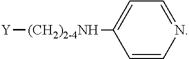

Embodiment 49. The ionizable cationic lipid of any one of embodiments 1 to 35, wherein X is

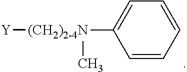

Embodiment 50. The ionizable cationic lipid of any one of embodiments 1 to 35, wherein X is

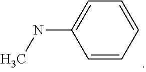

Embodiment 51. The ionizable cationic lipid of any one of embodiments 1 to 35, wherein X is

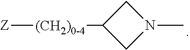

Embodiment 52. The ionizable cationic lipid of any one of embodiments 1 to 35, wherein X is

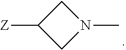

Embodiment 53. The ionizable cationic lipid of any one of embodiments 1 to 35, wherein X is

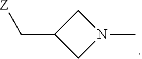

Embodiment 54. The ionizable cationic lipid of embodiment 1 to 35, wherein X is

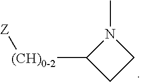

Embodiment 55. The ionizable cationic lipid of any one of embodiments 1 to 35, wherein X iS

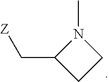

Embodiment 56. The ionizable cationic lipid of any of embodiments 1 to 35, wherein X is

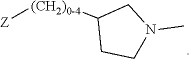

Embodiment 57. The ionizable cationic lipid of any one of embodiments 1 to 35, wherein X is

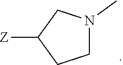

Embodiment 58. The ionizable cationic lipid of any one of embodiments 1 to 35, wherein X is

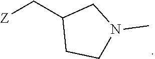

Embodiment 59. The ionizable cationic lipid of any of embodiments 1 to 35, wherein X is

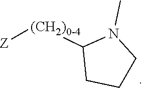

Embodiment 60. The ionizable cationic lipid of any one of embodiments 1 to 35, wherein X is

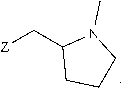

Embodiment 61. The ionizable cationic lipid of any one of embodiments 1 to 35, wherein X is

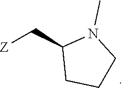

Embodiment 62. The ionizable cationic lipid of any one of embodiments 1 to 35, wherein X is

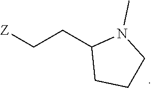

Embodiment 63. The ionizable cationic lipid of any one of embodiments 1 to 35, wherein X is

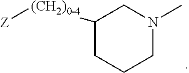

Embodiment 64. The ionizable cationic lipid of any one of embodiments 1 to 35, wherein X is

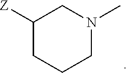

Embodiment 65. The ionizable cationic lipid of any one of embodiments 1 to 35, wherein X is

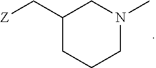

Embodiment 66. The ionizable cationic lipid of any of embodiments 1 to 35, wherein X is

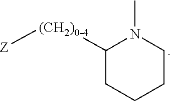

Embodiment 67. The ionizable cationic lipid of any one of embodiments 1 to 35, wherein X is

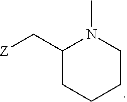

Embodiment 68. The ionizable cationic lipid of any one of embodiments 1 to 35, wherein X is

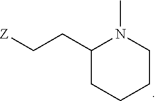

Embodiment 69. The ionizable cationic lipid of any one of embodiments 1 to 35, wherein X is

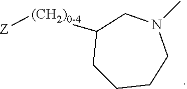

Embodiment 70. The ionizable cationic lipid of any one of embodiments 1 to 35, wherein X is

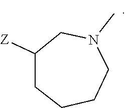

Embodiment 71. The ionizable cationic lipid of any one of embodiments 1 to 35, wherein X is

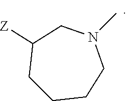

Embodiment 72. The ionizable cationic lipid of any one of embodiments 1 to 35, wherein X is

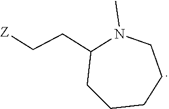

Embodiment 73. The ionizable cationic lipid of any of embodiments 1 to 35, wherein X is

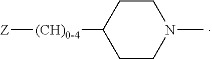

Embodiment 74. The ionizable cationic lipid of any one of embodiments 1 to 35, wherein X is

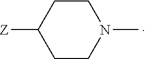

Embodiment 75. The ionizable cationic lipid of any one of embodiments 1 to 35, wherein X is

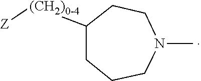

Embodiment 76. The ionizable cationic lipid of any one of embodiments 1 to 35, wherein X Z is

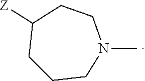

Embodiment 77. The ionizable cationic lipid of any one of embodiments 1 to 49 wherein Y is O.

Embodiment 78. The ionizable cationic lipid of any one of embodiments 1 to 49 wherein Y is S.

Embodiment 79. The ionizable cationic lipid of any one of embodiments 1 to 35 or 51-76, wherein Z is O.

Embodiment 80. The ionizable cationic lipid of embodiment 1 having the structure CICL-207:

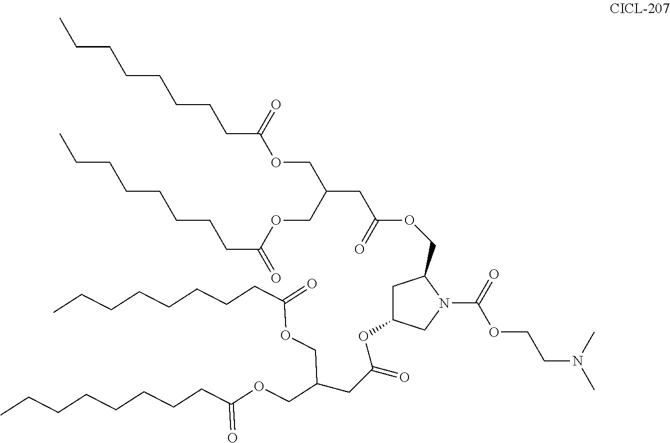 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)OCCN(C)C]

or a racemate or other mixture comprising CICL-225.

Embodiment 81. The ionizable cationic lipid of embodiment 1 having the structure CICL-215:

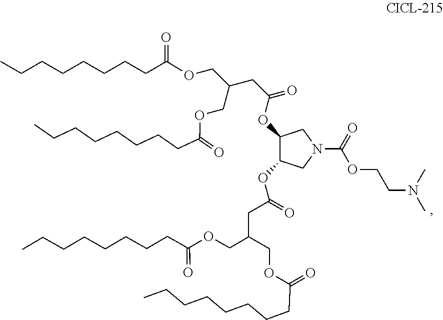 [SMILES: CCCCCCCCC(=O)OCC(COC(=O)CCCCCCCC)CC(=O)O[C@H]1CN(C(=O)OCCN(C)C)C[C@@H]1OC(=O)CC(COC(=O)CCCCCCCC)COC(=O)CCCCCCCC]

its enantiomer or a racemate, or other mixture comprising CICL-216.

Embodiment 82. The ionizable cationic lipid of embodiment 1 having the structure CICL-216:

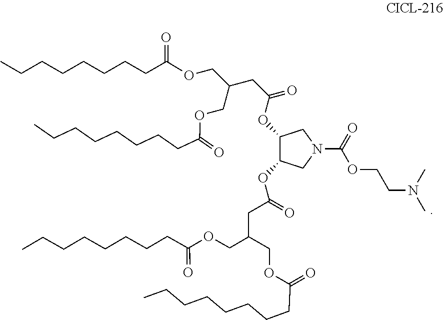 [SMILES: CCCCCCCCC(=O)OCC(COC(=O)CCCCCCCC)CC(=O)O[C@H]1CN(C(=O)OCCN(C)C)C[C@H]1OC(=O)CC(COC(=O)CCCCCCCC)COC(=O)CCCCCCCC]

Embodiment 83. The ionizable cationic lipid of embodiment 1 having the structure CICL-217:

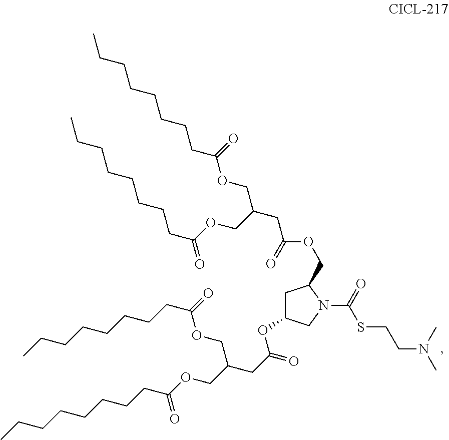 [SMILES: CCCCCCCCC(=O)OCC(COC(=O)CCCCCCCC)CC(=O)OC[C@@H]1C[C@@H](OC(=O)CC(COC(=O)CCCCCCCC)COC(=O)CCCCCCCC)CN1C(=O)SCCN(C)C]

its enantiomer, a racemate or any other mixture thereof.

Embodiment 84. The ionizable cationic lipid of embodiment 1 having the structure CICL-218:

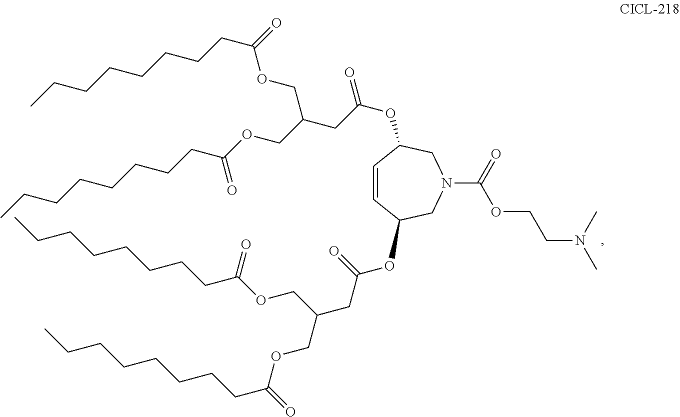

as a racemate, any other mixture of enantiomers, or either individual enantiomer.

Embodiment 85. The ionizable cationic lipid of embodiment 1 having the structure CICL-219:

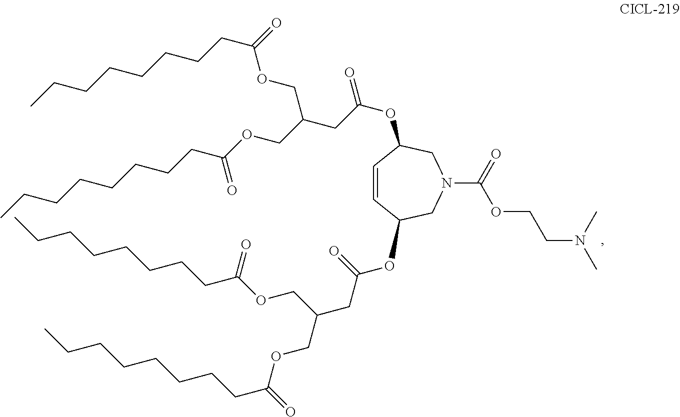

Embodiment 86. The ionizable cationic lipid of embodiment 1 having the structure CICL-220:

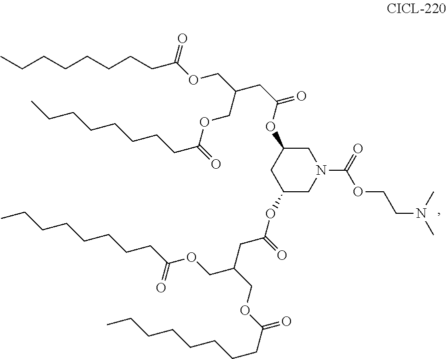 [SMILES: CCCCCCCCC(=O)OCC(COC(=O)CCCCCCCC)CC(=O)O[C@@H]1C[C@@H](OC(=O)CC(COC(=O)CCCCCCCC)COC(=O)CCCCCCCC)CN(C(=O)OCCN(C)C)C1]

its enantiomer, a racemate, any other mixture of enantiomers.

Embodiment 87. The ionizable cationic lipid of embodiment 1 having the structure CICL-221:

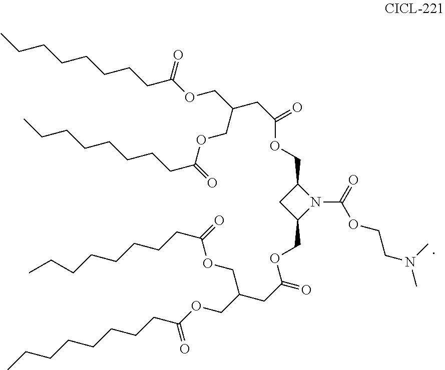 [SMILES: CCCCCCCCC(=O)OCC(COC(=O)CCCCCCCC)CC(=O)OC[C@@H]1C[C@H](COC(=O)CC(COC(=O)CCCCCCCC)COC(=O)CCCCCCCC)N1C(=O)OCCN(C)C]

Embodiment 88. The ionizable cationic lipid of embodiment 1 having the structure CICL-222:

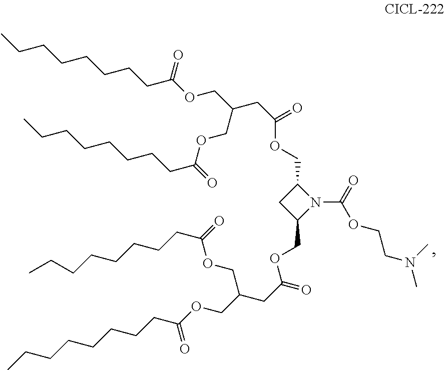 [SMILES: CCCCCCCCC(=O)OCC(COC(=O)CCCCCCCC)CC(=O)OC[C@H]1C[C@H](COC(=O)CC(COC(=O)CCCCCCCC)COC(=O)CCCCCCCC)N1C(=O)OCCN(C)C]

as a racemate, any other mixture of enantiomers, or either individual enantiomer.

Embodiment 89. The ionizable cationic lipid of embodiment 1 having the structure CICL-223:

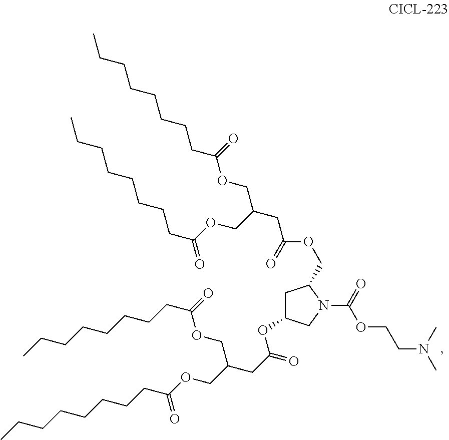 [SMILES: CCCCCCCCC(=O)OCC(COC(=O)CCCCCCCC)CC(=O)OC[C@H]1C[C@@H](OC(=O)CC(COC(=O)CCCCCCCC)COC(=O)CCCCCCCC)CN1C(=O)OCCN(C)C]

or a racemate or other mixture comprising CICL-224.

Embodiment 90. The ionizable cationic lipid of embodiment 1 having the structure CICL-224:

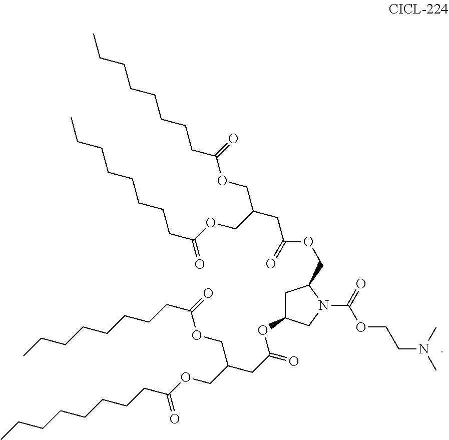 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)OCCN(C)C]

Embodiment 91. The ionizable cationic lipid of embodiment 1 having the structure CICL-225:

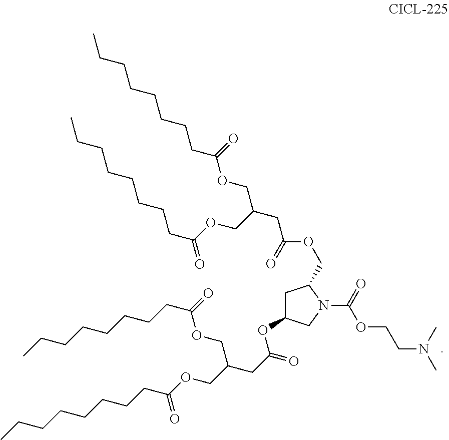 [SMILES: CCCCCCCCC(=O)OCC(COC(=O)CCCCCCCC)CC(=O)OC[C@H]1C[C@H](OC(=O)CC(COC(=O)CCCCCCCC)COC(=O)CCCCCCCC)CN1C(=O)OCCN(C)C]

Embodiment 92. The ionizable lipid of embodiment 1 having the structure CICL-238:

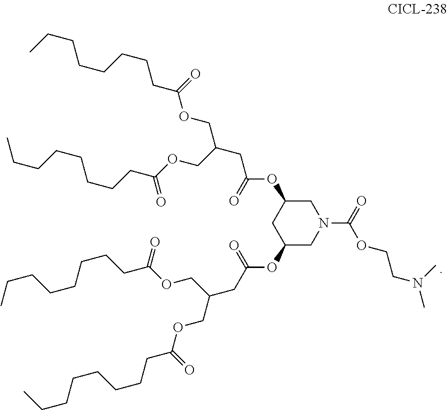 [SMILES: CCCCCCCCC(=O)OCC(COC(=O)CCCCCCCC)CC(=O)O[C@@H]1C[C@H](OC(=O)CC(COC(=O)CCCCCCCC)COC(=O)CCCCCCCC)CN(C(=O)OCCN(C)C)C1]

Embodiment 93. The ionizable cationic lipid of embodiment 1 having the structure CICL-239:

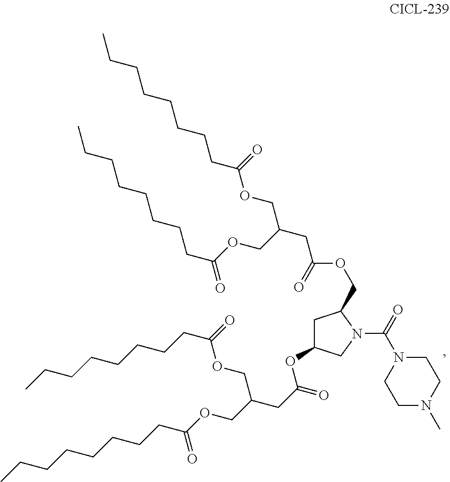 [SMILES: CCCCCCCCC(=O)OCC(COC(=O)CCCCCCCC)CC(=O)OC[C@@H]1C[C@H](OC(=O)CC(COC(=O)CCCCCCCC)COC(=O)CCCCCCCC)CN1C(=O)N1CCN(C)CC1]

its enantiomer, or a racemate or any other mixture thereof.

Embodiment 94. The ionizable cationic lipid of embodiment 1 having the structure CICL-242:

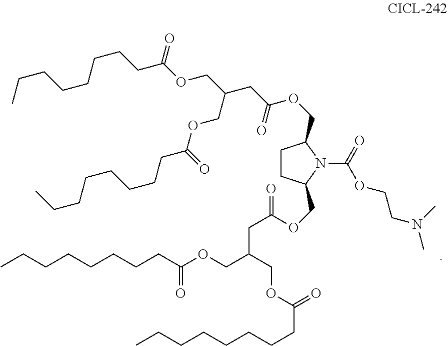 [SMILES: CCCCCCCCC(=O)OCC(COC(=O)CCCCCCCC)CC(=O)OC[C@H]1CC[C@@H](COC(=O)CC(COC(=O)CCCCCCCC)COC(=O)CCCCCCCC)N1C(=O)OCCN(C)C]

Embodiment 95. The ionizable cationic lipid of embodiment 1 having the structure CICL-243:

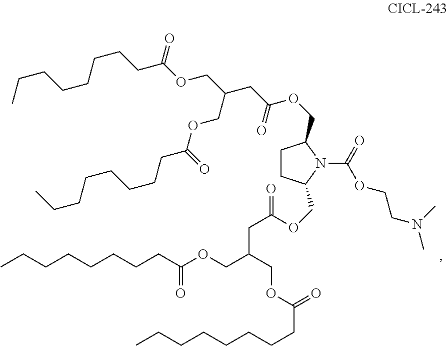 [SMILES: CCCCCCCCC(=O)OCC(COC(=O)CCCCCCCC)CC(=O)OC[C@@H]1CC[C@@H](COC(=O)CC(COC(=O)CCCCCCCC)COC(=O)CCCCCCCC)N1C(=O)OCCN(C)C]

its enantiomer, or a racemate or any other mixture thereof.

Embodiment 96. The ionizable cationic lipid of embodiment 1 having the structure CICL-244:

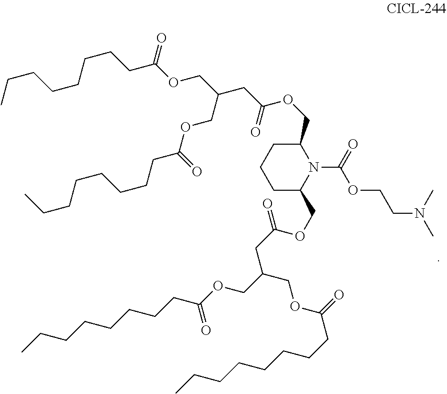 [SMILES: CCCCCCCCC(=O)OCC(COC(=O)CCCCCCCC)CC(=O)OC[C@H]1CCC[C@@H](COC(=O)CC(COC(=O)CCCCCCCC)COC(=O)CCCCCCCC)N1C(=O)OCCN(C)C]

Embodiment 97. The ionizable lipid of embodiment 1 having the structure CICL-245:

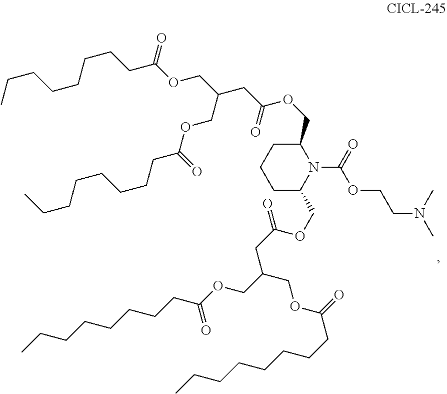 [SMILES: CCCCCCCCC(=O)OCC(COC(=O)CCCCCCCC)CC(=O)OC[C@@H]1CCC[C@@H](COC(=O)CC(COC(=O)CCCCCCCC)COC(=O)CCCCCCCC)N1C(=O)OCCN(C)C]

a racemate, any other mixture of enantiomers, or either individual enantiomer.

Embodiment 98. The ionizable cationic lipid of embodiment 1 having the structure CICL-246:

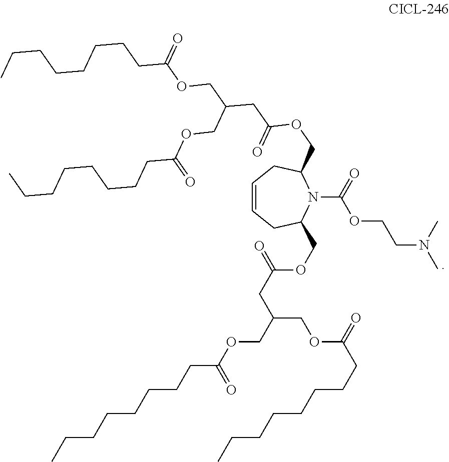 [SMILES: CCCCCCCCC(=O)OCC(COC(=O)CCCCCCCC)CC(=O)OC[C@H]1CC=CC[C@@H](COC(=O)CC(COC(=O)CCCCCCCC)COC(=O)CCCCCCCC)N1C(=O)OCCN(C)C]

Embodiment 99. The ionizable cationic lipid of embodiment 1 having the structure CICL-247:

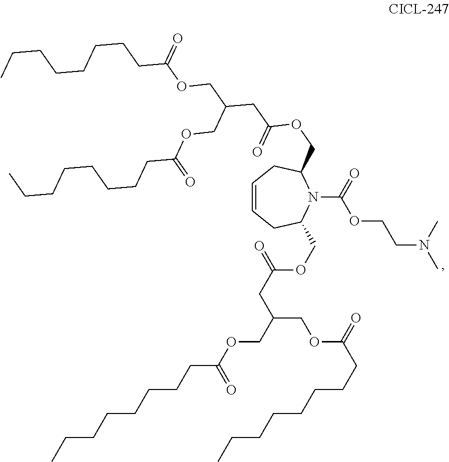 [SMILES: CCCCCCCCC(=O)OCC(COC(=O)CCCCCCCC)CC(=O)OC[C@@H]1CC=CC[C@@H](COC(=O)CC(COC(=O)CCCCCCCC)COC(=O)CCCCCCCC)N1C(=O)OCCN(C)C]

a racemate, any other mixture of enantiomers, or either individual enantiomer.

Embodiment 100. The ionizable cationic lipid of embodiment 1 having the structure CICL-248: CICL-248

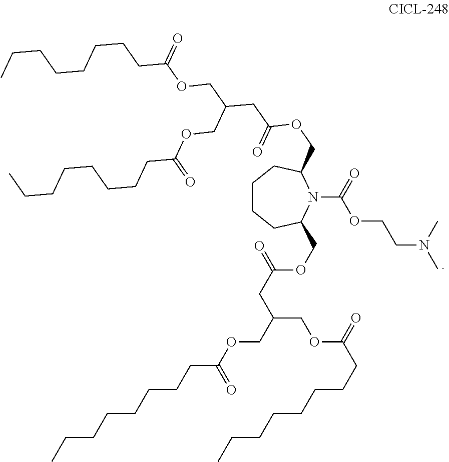 [SMILES: CCCCCCCCC(=O)OCC(COC(=O)CCCCCCCC)CC(=O)OC[C@H]1CCCC[C@@H](COC(=O)CC(COC(=O)CCCCCCCC)COC(=O)CCCCCCCC)N1C(=O)OCCN(C)C]

Embodiment 101. The ionizable cationic lipid of embodiment 1 having the structure CICL-249:

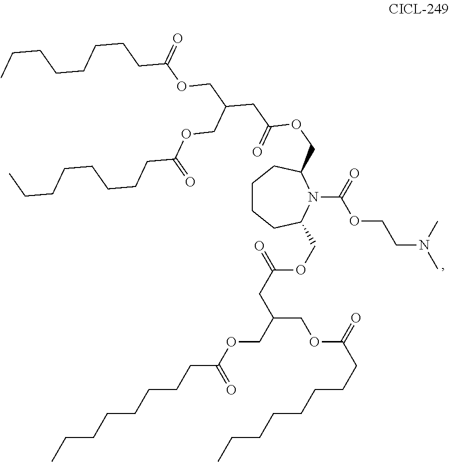 [SMILES: CCCCCCCCC(=O)OCC(COC(=O)CCCCCCCC)CC(=O)OC[C@@H]1CCCC[C@@H](COC(=O)CC(COC(=O)CCCCCCCC)COC(=O)CCCCCCCC)N1C(=O)OCCN(C)C]

a racemate, any other mixture of enantiomers, or either individual enantiomer.

Embodiment 102. The ionizable cationic lipid of embodiment 1 having a structure as shown in Table 4.

Embodiment 103. The ionizable cationic lipid of embodiment 1 having a structure as shown in Table 5.

Embodiment 104. The ionizable cationic lipid of embodiment 1 having a structure as shown in Table 6.

Embodiment 105. The ionizable cationic lipid of embodiment 1 having a structure as shown in Table 11.

Embodiment 106. The ionizable cationic lipid of embodiment 1 having a structure as shown in Table 12.

Embodiment 107. The ionizable cationic lipid of any one of embodiments 1 to 106 having a -pKa (calculated pKa) in the range of from about 6, 7, or 8 to about 9, 10, or 11.

Embodiment 108. The ionizable cationic lipid of any one of embodiments 1 to 106 having a c-pKa ranging from about 6 to about 10, about 7 to about 10, about 8 to about 10, about 8 to about 9, 6 to 10, 7 to 10, 8 to 10, or 8 to 9.

Embodiment 109. The ionizable cationic lipid of any one of embodiments 1 to 106 having a c-pKa ranging from about 8.2 to about 9.0 or from 8.2 to 9.0.

Embodiment 110. The ionizable cationic lipid of any one of embodiments 1 to 106 having a c-pKa ranging from about 8.4 to about 8.7 or from 8.4 to 8.7.

Embodiment 111. The ionizable cationic lipid of any one of embodiments 1 to 110 having a c Log D ranging from about 9 to about 18, for example, ranging from about 10 to about 18, or about 10 to about 16, to about 10 to about 14, or about 11 to about 18, or about 11 to about 15, or about 11 to about 14.

Embodiment 112. The ionizable cationic lipid of any one of embodiments 1 to 110 having a c Log D ranging from 9 to 18, for example, ranging from 10 to 18, or 10 to 16, to 10 to 14, or 11 to 18, or 11 to 15, or 11 to 14.

Embodiment 113. The ionizable cationic lipid of any one of embodiments 1 to 110 having a c Log D ranging from about 13.6 to about 14.4 or from 13.6 to 14.4.

Embodiment 114. The ionizable cationic lipid of any one of embodiments 1 to 110 having a c-pKa ranging from about 8 to about 11 or from 8 to 11 and a c Log D ranging from about 9 to about 18 or from 9 to 18.

Embodiment 115. The ionizable cationic lipid of any of embodiments 1 to 110 having a c-pKa ranging from about 8.4 to about 8.7 or from 8.4 to 8.7 and c Log D ranging from about 13.6 to about 14.4 or from 13.6 to 14.4.

Embodiment 116. The ionizable cationic lipid of any one of embodiments 1 to 110 having a c Log D is about 9, about 10, about 11, about 12, about 13, about 14, about 15, about 16, about 17, or about 18.

Embodiment 117. A lipid nanoparticle (LNP) or targeted lipid nanoparticle (tLNP), comprising at least one ionizable cationic lipid of any one of embodiments 1-116.

Embodiment 118. The LNP or tLNP of embodiment 117, further comprising one or more of a phospholipid, a sterol, a co-lipid, a PEG-lipid, or combinations thereof.

Embodiment 119. The LNP or tLNP of embodiment 118, comprising an unfunctionalized PEG-lipid.

Embodiment 120. The LNP or tLNP of embodiment 118 or embodiment 119, comprising a functionalized PEG-lipid.

Embodiment 121. The tLNP of embodiment 120, wherein the functionalized PEG-lipid has been conjugated with a binding moiety.

Embodiment 122. The tLNP of embodiment 120 or 121, wherein the binding moiety comprises an antigen binding domain of an antibody.

Embodiment 123. The tLNP of embodiment 120 or 121, wherein the binding moiety comprises an antigen, a ligand-binding domain of a receptor, or a receptor ligand.

Embodiment 124. The LNP or tLNP of any one of embodiments 118-123, comprising at least one phospholipid, wherein the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof.

Embodiment 125. The LNP or tLNP of any one of embodiments 118-124, comprising at least one sterol, wherein the sterol comprises cholesterol, campesterol, sitosterol, stigmasterol, or combinations thereof.

Embodiment 126. The LNP or tLNP of any one of embodiments 118-125, comprising at least one co-lipid, wherein the co-lipid comprises cholesterol hemisuccinate (CHEMS) or a quaternary ammonium headgroup containing lipid.

Embodiment 127. The LNP or tLNP of embodiment 126, wherein the quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 30-(N—(N′,N′-Dimethylaminoethane)carbamoyl)cholesterol (DC-Chol), or combinations thereof.

Embodiment 128. The LNP or tLNP of any one of embodiments 119-127, comprising at least one funcationalized or unfuncationalized PEG-lipid, wherein the functionalized or unfunctionalized PEG-lipid comprises a PEG moiety of 1000-5000 Da molecular weight (MW).

Embodiment 129. The LNP or tLNP of any one of embodiments 119-128, comprising at least one funcationalized or unfuncationalized PEG-lipid, wherein the functionalized or unfunctionalized PEG-lipid comprises fatty acids with a fatty acid chain length of C₁₄-C₁ₐ.

Embodiment 130. The LNP or tLNP of any one of embodiments 119-129, comprising at least one funcationalized or unfuncationalized PEG-lipid, wherein the functionalized or unfunctionalized PEG-lipid comprises DMG-PEG2000 (1,2-dimyristoyl-rglycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethyleneglycol-2000), DOPE-PEG2000 (1,2-dioleoyl-glycero-3-phosphoethanolamine-3-methoxypolyethylene glycol-2000), or combinations thereof.

Embodiment 131. The LNP or tLNP of any one of embodiments 117-130, wherein the at least one ionizable cationic lipid is present in an amount in the range from 40 to 65 mol %.

Embodiment 132. The LNP or tLNP of any one of embodiments 124-131, comprising a phospholipid in an amount in the range from 7 to 30 mol %.

Embodiment 133. The LNP or tLNP of any one of embodiments 125-132, comprising a sterol in an amount in the range from 20 to 45 mol %.

Embodiment 134. The LNP or tLNP of any one of embodiments 126-133, comprising at least one co-lipid in an amount in the range from 1 to 30 mol %.

Embodiment 135. The LNP or tLNP of any one of embodiments 118, or 121-134, comprising at least one unfunctionalized PEG-lipid in an amount in the range from 0.1 to 5 mol %.

Embodiment 136. The LNP or tLNP of any one of embodiments 118-135, comprising at least one functionalized PEG-lipid in an amount in the range from 0.1 to 5 mol %.

Embodiment 137. The LNP or tLNP of any one of embodiments 117-136, further comprising a biologically active payload nucleic acid.

Embodiment 138. The LNP or tLNP of embodiment 137, wherein the weight ratio of total lipid to nucleic acid is 10:1 to 50:1.

Embodiment 139. The LNP or tLNP of embodiment 137, wherein the N/P ratio is from 3 to 9.

Embodiment 140. The LNP or tLNP of any one of embodiments 137-139, wherein the nucleic acid payload is mRNA.

Embodiment 141. The tLNP of any one of embodiments 120-122 or 124-140, wherein the binding moiety is a whole antibody and the ratio of antibody to nucleic acid is from about 0.3 to about 1.0 (w/w).

Embodiment 142. The tLNP of any one of embodiment 118-141, wherein the tLNP is targeted to aT cell.

Embodiment 143. The tLNP of any one of embodiments 118-141, wherein the tLNP is targeted to a CD8+ T cell.

Embodiment 144. The tLNP of any one of embodiments 118-141, wherein the tLNP is targeted to an HSC.

Embodiment 145. The tLNP of any one of embodiments 118-141, wherein the tLNP is targeted to a CD117+ cell.

Embodiment 146. A method of delivering a nucleic acid into a cell comprising contacting the cell with the LNP or tLNP of any one of embodiments 137 to 145.

Embodiment 147. A lipid having the structure of formula M2-1,

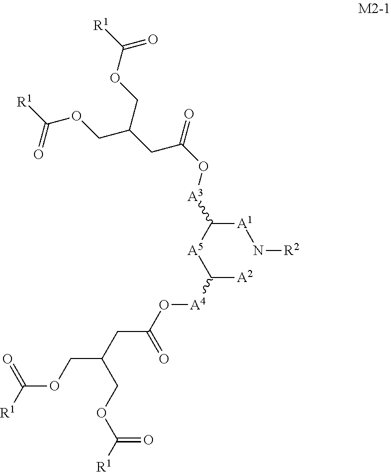

wherein
each R¹ is independently selected from C₇-C₁₁ alkyl or C₇-C₁₁ alkenyl;
R² is H of a protecting group;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
wavy bonds indicate that any relative or absolute stereo-configuration of the corresponding ring atom can be assumed.

Embodiment 148. The lipid of embodiment 147, wherein R¹ is as described in any one of embodiments 14-25.

Embodiment 149. The lipid of embodiments 147 or 148, wherein A¹, A², A³, A⁴, and A⁵ are as described in any one of embodiments 2 to 13.

Embodiment 150. The lipid of any one of embodiments 147 to 149, wherein R² is H.

Embodiment 151. The lipid any one of embodiments 147 to 150, wherein R² is a protecting group (e.g., t-butoxycarbonyl (BOC), benzyloxycarbonyl (Cbz), or a trimethylsilylethoxycarbonyl moiety.

Embodiment 152. A lipid having the structure of formula M2-2,

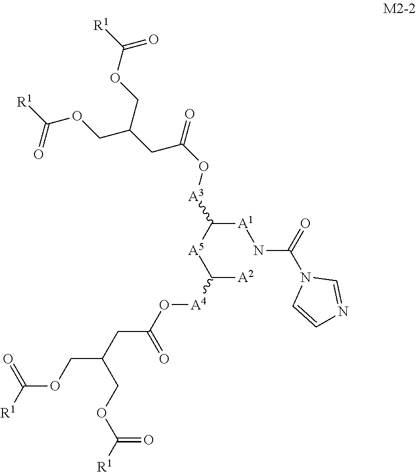

each R¹ is independently selected from C₇-C₁₁ alkyl or C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and wavy bonds indicate that any relative or absolute stereo-configuration of the corresponding ring atom can be assumed.

Embodiment 153. The lipid of embodiment 152, wherein R¹ is as described in any one of embodiments 14-25.

Embodiment 154. The lipid of embodiments 152 or 153, wherein A¹, A², A³, A⁴, and A⁵ are as described in any one of embodiments 2-13.

Embodiment 155. A synthesis method of an ionizable cationic lipid of formula M2, the method comprising:
providing a lipid of formula M2-1;
reacting the lipid of formula M2-1 with carbonyldiimidazole to provide a lipid of formula M2-2; and
coupling the lipid of formula M2-2 with H—X in the presence of a base to provide the ionizable cationic lipid of formula M2;
wherein H—X is selected from the group consisting of: of

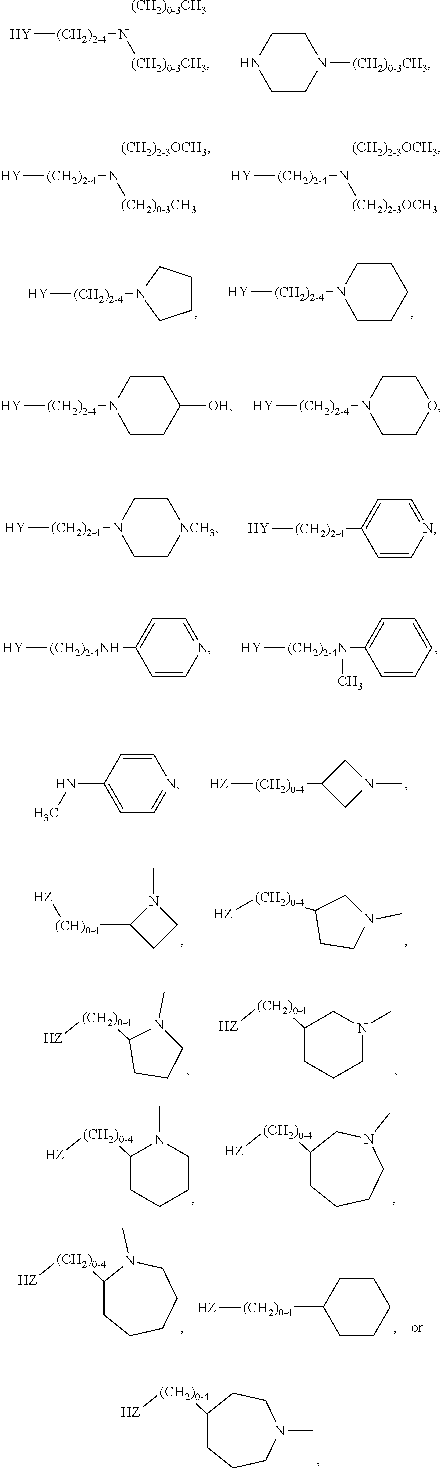

wherein Y is O, S, NH, of NCH₃ and Z is O, NH, NCH₃

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that the combination of these measures or features cannot be used. Any reference signs in the claims should not be construed as limiting the scope.

APPENDIX A

| | | Commercial Source(s) (if available) | | |
|---|---|---|---|---|
| | Antigen/ | | Supplier and | Publication |
| Antibody | Target | Product Name | Catalog Number | Information |
| 1.5.3 | CD20 | | | Gazit-Bornstein et |
| | | | | al., 2010, Invest. |
| | | | | New Drugs 28: 561- |
| | | | | 574. |
| 1C5 | CD117 | c-Kit/CD117 Antibody | ThermoFisher, | |
| | | (1C5) | #MA5-15894 | |
| 1D1C2 | IL-21Rα | IL21R Monoclonal | ThermoFisher, | |
| | | Antibody (1D1C2) | #66319-1-IG | |
| 1F5 | CD20 | Anti-CD20 [1F5] | Absolute | |
| | | | Antibody, | |
| | | | #Ab01133 | |
| 1mono2A6 | CD2 | Ellis Reinherz, Dana-Farber Cancer | | |
| (T113) | | Institute, Boston, MA | | |
| 1OLD2-4C1 | CD2 | Ellis Reinherz, Dana-Farber Cancer | | US 2022/0073639 |
| (T112) | | Institute, Boston, MA | | (SEQ ID NO. 51 & |
| | | | | 52) |
| 11B1H1B4 | CD150 | CD150 Monoclonal | Fisher Scientific, | |
| | | Antibody (11B1H1B4), | #16384654 | |
| | | Invitrogen ™ | | |
| 158-4D3 | CD45 | CD45 Monoclonal | ThermoFisher, | |
| | | Antibody (158-4D3) | #MA1-7636 | |
| 190-2F2.5 | CD45RO | CD45RO (T-Cell | ThermoFisher, | |
| | | Marker) Monoclonal | #5788-MSM12- | |
| | | Antibody (190-2F2.5) | P0 | |
| 2.1.2 | CD20 | Anti-Human MS4A1 | Creative Biolabs, | US 2007/0014720 |
| | | Recombinant Antibody | #TAB-1645CL | |
| | | (2.1.2) | | |
| 22HCLC | CD117 | Phospho-c-Kit (Tyr703) | Fisher Scientific, | |
| | | Recombinant | #PI710762 | |
| | | Polyclonal Antibody | | |
| | | (22HCLC), Invitrogen ™ | | |
| 2A9 | SSEA-3 | Anti-SSEA-3/GalGb4 | AntibodySystem, | |
| | | Antibody (2A9) | #RGK27501 | |
| 2A10D6 | FCRL5 | CD307e (FcRL5) | ThermoFisher, | |
| | | Monoclonal Antibody | #MA5-48789 | |
| 2A10H7 | FCRL5 | CD307e (FcRL5) | ThermoFisher, | |
| | | Monoclonal Antibody | #MA5-38483 | |
| 2A11D6 | CD43 | CD43 Monoclonal | ThermoFisher, | |
| | | Antibody (2A11D6) | #66224-1-IG | |
| 2B8 | CD117 | CD117 (c-Kit) | ThermoFisher, | |
| | | Monoclonal Antibody | #14-1171-82 | |
| | | (2B8), eBioscience ™ | | |
| 2B8/BM | CD117 | CD117 (c-Kit) | ThermoFisher, | |
| | | Monoclonal Antibody | #MA5-28270 | |
| | | (2B8/BM) | | |
| 2D4-C9-F1 | Sca-1 | Mouse anti-Casp3- | Biocat, #130- | |
| | | 1(138-157) (2D4-C9-F1) | 10623-20-RB | |
| 2E10 | CD34 | available from multiple sources, including: | | |
| | | Anti-CD34 [2E10] | Absolute | |
| | | | Antibody, | |
| | | | #Ab01129 | |
| | | Human Anti-CD34 | Creative Biolabs, | |
| | | Recombinant Antibody | #FAMAB- | |
| | | (clone 2E10) | 1049CQ | |
| 2ST8.5H7 | CD8β | available from multiple sources, including: | | |
| | | CD8 Beta antibody \| | Bio-Rad, | |
| | | 2ST8.5H7 | #MCA1723 | |
| | | CD8 Antibody | Novus | |
| | | (2ST8.5H7) | Biologicals, | |
| | | | #NB100-65928 | |
| 307307 | FCRL5 | CD307e (FcRL5) | ThermoFisher, | |
| | | Monoclonal Antibody | #MA5-24020 | |
| 3A1E | CD7 | CD7 Chimeric | ThermoFisher, | |
| | | Recombinant Rabbit | #MHCD0800 | |
| | | Monoclonal Antibody | | |
| | | (3A1E) | | |
| 3A1F | CD7 | Anti-CD7 [3A1F] | Absolute | |
| | | | Antibody, | |
| | | | #Ab03052 | |
| 3B5 | CD8 | available from multiple sources, including: | | |
| | | CD8 Monoclonal | ThermoFisher, | |
| | | Antibody (3B5) | #MHCD0800 | |
| | | | Aviva Systems | |
| | | | Biology, | |
| | | | #OAAI00153 | |
| 3B8-2C2 | CD48 | CD48 Monoclonal | ThermoFisher, | |
| | | Antibody (3B8-2C2) | #H00000962-M01 | |
| 3F7 | CD73 | | | Qiao et al., 2019, |
| | | | | Int. J. Mol. Sci. 20 |
| | | | | (5): 1057. |
| 3F7C6 | CD68 | CD68 monoclonal | Abnova, | |
| | | antibody, clone 3F7C6 | #MAB12263 | |
| 3F7D3 | CD68 | available from multiple sources, including: | | |
| | | CD68 monoclonal | Abnova, | |
| | | antibody, clone 3F7D3 | #MAB12260 | |
| | | Anti-CD68 antibody | Abcam, | |
| | | [3F7D3] | #ab201973 | |
| 3F8BiAb | GD2 | | | Yankelevich et al., |
| | CD3 | | | 2012, Pediatr. Blood |
| | | | | Cancer 59(7): 1198- |
| | | | | 1205. |
| 3G9-2D2 | CD205 | | | Cheong et al., 2010, |
| | | | | Blood 116 (19): |
| | | | | 3828-3838. |
| 3H420 | SSEA-3 | SSEA-3 Antibody | Santa Cruz | |
| | | (3H420) | Biotechnology, | |
| | | | #sc-73066 | |
| 3T4-8B5 | CD2 | Ellis Reinherz, Dana-Farber Cancer | | |
| (T111) | | Institute, Boston, MA | | |
| 4C5 | Stro-4 | | | Thomaidou and |
| | | | | Patsavoudi, 1993, |
| | | | | Neuroscience 53: |
| | | | | 813-827. |
| 4G5 | FAP | | | WO 2021/061708; |
| | | | | WO 2021/061778 |
| 4G7 | CD19 | | | Meeker et al., 1984, |
| | | | | Hybridoma 3: 305- |
| | | | | 320. |
| 4KB5 | CD45RA | Anti-CD45RA antibody | Abcam, #ab755 | |
| | | [4KB5] | | |
| 47G4 | CD19 | | | US 2010/0104509 |
| 51.1 | CD8 | Anti-CD8 [51.1] | Absolute | |
| | | | Antibody, | |
| | | | #Ab04357 | |
| 53H8L29 | CD117 | Phospho-c-Kit (Tyr703) | ThermoFisher, | |
| | | Recombinant Rabbit | #700135 | |
| | | Monoclonal Antibody | | |
| | | (53H8L29) | | |
| 5B4G1 | CD150 | SLAM/CD150 | ThermoFisher, | |
| | | Monoclonal Antibody | #60043-1-IG | |
| | | (5B4G1) | | |
| 5B12 | CD34 | available from multiple sources, including: | | |
| | | Anti-CD34 [5B120 | Absolute | |
| | | | Antibody, | |
| | | | #Ab00422 | |
| | | Human Anti-CD34 | Creative Biolabs, | |
| | | Recombinant Antibody | #FAMAB- | |
| | | (clone 5B12) | 1047CQ | |
| 5D7 | CD5 | CD5 Monoclonal | ThermoFisher, | |
| | | Antibody (CD5-5D7), | #MHCD0505 | |
| | | APC | | |
| 5E3E1 | IL-15Rα | IL-15RA Monoclonal | ThermoFisher, | |
| | | Antibody (5E3E1) | #MHCD0505 | |
| 5E10 | CD90 | available from multiple sources, including: | | |
| | | CD90 (Thy-1) | ThermoFisher, | |
| | | Monoclonal Antibody | #14-0909-82 | |
| | | (eBio5E10 (5E10)), | | |
| | | eBioscience ™ | | |
| | | Anti-Human CD90 | Stemcell | |
| | | Antibody, Clone 5E10 | Technologies, | |
| | | | #60045 | |
| 509F6 | FCRL5 | CD307e (FcRL5) | ThermoFisher, | |
| | | Monoclonal Antibody | #50-3078-42 | |
| | | (509F6), eFluor ™ 660, | | |
| | | eBioscience ™ | | |
| 6D276 | CD29 | Anti-CD49b, CD29 | VWR, | |
| | CD49b | Complex Mouse | #USBIC2404- | |
| | | Monoclonal Antibody | 07W | |
| | | [clone: 6D276] | | |
| 6D9 | GPRC5D | Anti-GPRC5D (6D9) | Creative Biolabs, | |
| | | h(41BB-CD3ζ) CAR, | #CAR-LY1362 | |
| | | pCDCAR1 | | |
| 6F2 | CD117 | KIT Monoclonal | ThermoFisher, | |
| | | Antibody (6F2) | #H00003815-M02 | |
| 7D11 | FCRL5 | Anti-FcRH5 (clone | Creative Biolabs, | |
| | | 7D11)-SMCC-DM1 | #ADC-161CL | |
| | | ADC | | |
| 7E1 | CD48 | CD48 Recombinant | ThermoFisher, | |
| | | Rabbit Monoclonal | #MA5-38301 | |
| | | Antibody (7E1) | | |
| 8A7 | SSEA-3 | Anti-SSEA-3/GalGb4 | AntibodySystem, | |
| | | Antibody (8A7) | #RGK27504 | |
| 8D7 | CD117 | c-Kit Monoclonal | ThermoFisher, | |
| | | Antibody (8D7) | #MA5-15327 | |
| 9.1 | CD2 | Peter Linsley, Benaroya Research | | Connelly et al., |
| | | Institute, Seattle, WA | | 1998, Int. Immunol., |
| | | | | 10 (12): 1863-1872. |
| 9.6 | CD2 | Peter Linsley, Benaroya Research | | Connelly et al., |
| | | Institute, Seattle, WA | | 1998, Int. Immunol., |
| | | | | 10 (12): 1863-1872. |
| 9C5 | CD34 | | | U.S. Pat. No. 8,399,249 |
| 9D1 | CD150 | CD150 Monoclonal | ThermoFisher, | |
| | | Antibody (9D1), PE, | #12-1501-82 | |
| | | eBioscience ™ | | |
| 10A8 | FCRL5 | Anti-FcRL5 (10A8)- | Creative Biolabs, | |
| | | SPDB-DM4 ADC | #ADC-117LCT | |
| 12G10 | CD29 | available from multiple sources, including: | | |
| | | CD29 (Stem Cell | ThermoFisher, | |
| | | Marker) Monoclonal | #3688-MSM2-PO | |
| | | Antibody (12G10) | | |
| | | CD29 antibody \| 12G10 | Bio-Rad, | |
| | | | #MCA2028 | |
| 13G9 | FCRL5 | Mouse Anti-FCRL5 | Creative Biolabs, | |
| | | Recombinant Antibody | #MOB-057CQ | |
| 17D8 | CD8 | CD8 Monoclonal | ThermoFisher, | |
| | | Antibody (17D8) | #MA1-12027 | |
| 18A5 | IL-21Rα | Human Anti-IL21R | Creative Biolabs, | |
| | | Recombinant Antibody | #HPAB-0668-CN | |
| 84-3C1 | CD43 | CD43 Monoclonal | ThermoFisher, | |
| | | Antibody (PE, | #12-0439-42 | |
| | | eBioscience ™) | | |
| 104D2 | CD117 | available from multiple sources, including: | | |
| | | Purified anti-human | BioLegend, | |
| | | CD117 (c-kit) Antibody | #313201 | |
| | | Anti-Human CD117 (c- | Stemcell Tech- | |
| | | Kit) Antibody, Clone | nologies, #60087 | |
| | | 104D2 | | |
| | | MOUSE ANTI HUMAN | Bio-Rad, | |
| | | CD117 | #MCA1841 | |
| | | c-Kit Monoclonal | ThermoFisher, | |
| | | Antibody (104D2) | #MA1-10072 | |
| 131-I- | CD45 | | | Seropian et al., |
| apamistamab | | | | 2023, Blood 142 |
| | | | | (Supplement 1): |
| | | | | 2159. |
| 131-I- | B7H3 | | | Kramer et al., 2022, |
| omburtamab | | | | J. Hematol. Oncol. |
| (131-I-8H9) | | | | 15: 165. |
| 177-Lu-DTPA- | B7H3 | | | Khatua et al., 2021, |
| omburtamab | | | | Ann. Oncol. 32 |
| | | | | (Suppl. 5): S528- |
| | | | | S529. |
| 156-4H9 | CD48 | CD48 Monoclonal | ThermoFisher, | |
| | | Antibody (eBio 156-4H9 | #14-0489-82 | |
| | | (156-4H9)) | | |
| | | eBioscience ™ | | |
| 293C3 | CD133 | available from multiple sources, including: | | |
| | | Anti-CD133 antibody | Abcam, | |
| | | [293C3] | #ab252553 | |
| | | CD133/2 Antibody, | Miltenyi Biotec, | |
| | | anti-human, pure | #130-090-851 | |
| 341 | CD48 | CD48 Recombinant | ThermoFisher, | |
| | | Rabbit Monoclonal | #MA5-29675 | |
| | | Antibody (341) | | |
| 514H12 | CD68 | CD68 Monoclonal | ThermoFisher, | |
| | | Antibody (514H12) | #MA1-80133 | |
| 1279B | SUSD2 | SUSD2 Antibody | Novus | |
| | | (1279B) | Biologicals, | |
| | | | #MAB90564 | |
| 2639B | IL-15Rα | Human IL-15R alpha | R&D Systems, | |
| | | Antibody | #MAB10900 | |
| A7R34 | CD127 | CD127 Monoclonal | ThermoFisher, | |
| | | Antibody (A7R34), | #14-1271-82 | |
| | | eBioscience ™ | | |
| A12 | CD150 | CD150 Monoclonal | ThermoFisher, | |
| (7D4) | | Antibody (A12 (7D4)), | #12-1509-42 | |
| | | PE, eBioscience ™ | | |
| AB75 | CD2 | available from multiple sources, including: | | |
| | | CD2 Monoclonal | ThermoFisher, | |
| | | Antibody (AB75) | #MA5-11373 | |
| | | Anti-CD2 Mouse | Avantor (VWR), | |
| | | Monoclonal Antibody | #GTX75367 | |
| | | [clone: AB75] | (89365-772) | |
| Ab85 | CD117 | | | WO 2019/084053 |
| ABL501 | LAG-3 | | | Park et al., 2021, |
| | PD-L1 | | | Cancer Res. 81 |
| | | | | (13_Supplement): |
| | | | | 1633. |
| ABM53F5 | CD68 | Monoclonal antibody to | Abeomics, #10- | |
| | | CD68 (Clone: | 4171 | |
| | | ABM53F5) | | |
| ABX-MA1 | CD146 | Anti-CD146 [ABX-MA1 | Absolute | |
| | | (3.19.1)] | Antibody, | |
| | | | #Ab02820 | |
| AC133 | CD133 | CD133/1 Antibody, | Miltenyi Biotec, | |
| | | anti-human, pure | #130-090-422 | |
| acazicolcept | CD28 | Acazicolcept | MedChemExpress, | |
| | | | #HY-P99420 | |
| ACK2 | CD117 | CD117 (c-Kit) | ThermoFisher, | |
| | | Monoclonal Antibody | #17-1172-82 | |
| | | (ACK2), APC, | | |
| | | eBioscience ™ | | |
| ACK4 | CD117 | c-Kit Monoclonal | ThermoFisher, | |
| | | Antibody (ACK4) | #MA1-70079 | |
| Adcitmer ® | CD56 | | | Esnault et al., 2022, |
| | | | | Br. J. Dermatol. 186 |
| | | | | (2): 295-306. |
| ADG106 | CD137 | | | Ma et al., 2024, Cell |
| | | | | Rep. Med. 5 (2): |
| | | | | 101414. |
| ADG116 | CTLA-4 | | | Park et al., 2022, J. |
| | | | | Immunother. Cancer |
| | | | | 10 (Suppl 2): A1- |
| | | | | A1603. |
| ADG126 | CTLA-4 | | | Richardson et al., |
| | | | | 2022, Ann. Oncol. |
| | | | | 33 (suppl 7): S882- |
| | | | | S883. |
| ADU-1604 | CTLA-4 | | | De Velasco Oria de |
| | | | | Rueda et al., 2023, |
| | | | | J. Immunother. |
| | | | | Cancer 11 (Suppl |
| | | | | 1): A1-A1731. |
| AFM13 | CD16a | Anti-FcgR3a/CD16a | Selleckchem, | |
| | | (AFM13) | #A2750 | |
| AGEN1181 | CTLA-4 | | | El-Khoueiry, et al., |
| | | | | 2021, J. |
| | | | | Immunother. Cancer |
| | | | | 9 (Suppl 2): A1- |
| | | | | A1054. |
| AK112 | PD-1 | Research Grade | AntibodySystem, | |
| | VEGF | Ivonescimab | #DHD12605 | |
| AK119 | CD73 | Research Grade | Cell Sciences, | |
| | | Dresbuxelimab | #DHD46603A | |
| AK129 | LAG-3 | | | Huang et al., 2022, |
| | | | | Cancer Res. 82 |
| | | | | (12_Supplement): |
| | | | | 5520. |
| alefacept | CD2 | Alefacept | MedChemExpres | |
| | | | s, #HY-P99429 | |
| AlexaFluor647 | IgG | available from multiple sources, including: | | |
| anti-hlgG Fc | | | | |
| detection | | Alexa Fluor ® 647 | Jackson | |
| | | AffiniPure ™ Goat Anti- | ImmunoResearch | |
| | | Human IgG, Fcγ | Laboratories, | |
| | | fragment specific | #109-605-098 | |
| antibody | | Human IgG Alexa | R&D Systems, | |
| | | Fluor ® 647-conjugated | #FAB110R | |
| | | Antibody | | |
| alirocumab | PCSK9 | available from multiple sources, including: | | |
| | | Alirocumab | MedChemExpress, | |
| | | | #HY-P9928 | |
| | | Alirocumab Biosimilar - | Proteogenix, | |
| | | Anti-PCSK9 mAb | #PX-TA1287 | |
| | | Alirocumab (anti- | SelleckChem, | |
| | | PCSK9) | #A2047 | |
| alnuctumab | BCMA | | | U.S. Pat. No. 10,683,369 |
| AMG-172 | CD70 | | | Massard et al., |
| | | | | 2019, Cancer |
| | | | | Chemother. |
| | | | | Pharmacol. |
| | | | | 83(6): 1057-63. |
| AMG 224 | BCMA | | | U.S. Pat. No. 9,243,058 |
| AMG 228 | GITR | | | Tran et al., 2017, J. |
| | | | | Clin. Oncol. 35 |
| | | | | (15_suppl): 2521. |
| AMG 404 | PD-1 | Zeluvalimab | MedChemExpress, | |
| | | | #HY-P99957 | |
| AMM22070N | Sca-1 | Caspase-3 Mouse mAb | Leading Biology, | |
| | | | #AMM22070N | |
| anti-BCMA CARs | BCMA | | | US 2020/0246381; |
| | | | | US 2020/0339699 |
| anti-CD3 FITC | CD3 | BD Pharmingen ™ | BD Biosciences, | |
| antibody | | FITC mouse anti- | #556611 | |
| conjugate | | human CD38 | | |
| anti-CD3 SP34-2 | CD3 | BD Pharmingen ™ | BD Biosciences, | |
| Alexa Fluor 700 | | Alexa Fluor ® 700 | #557917 | |
| conjugate | | mouse anti-human | | |
| antibody | | CD3 | | |
| anti-CD3 V450 | CD3 | available from multiple sources, including: | | |
| | | BD Horizon ™ V450 | BD Biosciences, | |
| | | Mouse Anti-Human | #560366 | |
| | | CD3 | | |
| | | violetFluor ™ 450 Anti- | Cytek, #75-0038- | |
| | | Human CD3 (UCHT1) | T025 | |
| anti-CD4 | CD4 | available from multiple sources, including: | | |
| (PerCP-Cy5.5) | | BD Pharmingen ™ | BD Biosciences, | |
| | | PerCP-Cy ™5.5 Mouse | #560650 | |
| | | Anti-Human CD4 | | |
| | | CD4 Monoclonal | ThermoFisher, | |
| | | Antibody (RM4-5), | #45-0042-82 | |
| | | PerCP-Cyanine5.5, | | |
| | | eBioscience ™ | | |
| anti-CD4 BV 421 | CD4 | Brilliant Violet 421 ™ | BioLegend, | |
| antibody | | anti-human CD4 | #317434 | |
| conjugate | | Antibody | | |
| anti-CD4 OKT4 | CD4 | Brilliant Violet 650 ™ | BioLegend, | |
| BV650 | | anti-human CD4 | #317436 | |
| conjugate | | Antibody | | |
| antibody | | | | |
| anti-CD8a | CD8α | BD Pharmingen ™ | BD Biosciences, | |
| (APC-H7) | | APC-H7 Mouse anti- | #560179 | |
| | | Human CD8 | | |
| anti-claudin 18.2 | CLDN18.2 | | | WO 2013/167259, |
| antibodies | | | | WO 2021/032157, |
| | | | | WO 2021/254481, |
| | | | | WO 2022/007808, |
| | | | | WO 2021/008463, |
| | | | | WO 2022/111616, |
| | | | | WO 2018/006882, |
| | | | | WO 2020/147321, |
| | | | | WO 2019/219089, |
| | | | | US 2020/0040101, |
| | | | | WO 2020/025792, |
| | | | | WO 2020/139956, |
| | | | | WO 2020/135201, |
| | | | | US 2024/0228610, |
| | | | | WO 2021/218874, |
| | | | | WO 2021/027850, |
| | | | | WO 2021/129765, |
| | | | | WO 2022/068854, |
| | | | | WO 2021/111003 |
| anti-FAP CARs | FAP | | | WO 2021/061778 |
| anti-FCRL5 CARS | FCRL5 | | | WO 2016/090337, |
| and ADCs | | | | WO 2017/096120, |
| | | | | WO 2022/263855, |
| | | | | WO 2024/047558, |
| | | | | Elkins et al., Mol. |
| | | | | Cancer Ther. |
| | | | | 11(10): 2222-2232 |
| | | | | (2012) |
| anti-G4S PE | G4S | G4S Linker (E7O2V) | Cell Signaling | |
| antibody | | Rabbit mAb (PE | Technology, | |
| | | Conjugate) | #38907 | |
| anti-human Fc | Fc | Brilliant Violet 421 ™ | BioLegend, | |
| secondary | | anti-human IgG Fc | #410704 | |
| antibody | | Antibody | | |
| (conjugated to | | | | |
| BV421 | | | | |
| fluorophore) | | | | |
| anti-scFv linker | scFv | MonoRab ™ Rabbit | GenScript, | |
| PE antibody | linker | Anti-scFv Cocktail [PE] | #A02285 | |
| anti-siglec-15 | Siglec-15 | | | U.S. Pat. No. 8,575,531 |
| antibody | | | | |
| anti-Whitlow | | Whitlow/218 Linker | Cell Signaling | |
| linker PE | | (E3U7Q) Rabbit mAb | Technology, | |
| antibody | | (PE Conjugate) | #62405 | |
| ANV419 | CD122 | | | Joerger et al., 2023, |
| | | | | J. Immunother. |
| | | | | Cancer 11 (11): |
| | | | | e007784. |
| ARC0672 | CD127 | CD127 Recombinant | ThermoFisher, | |
| | | Rabbit Monoclonal | #MA5-35152 | |
| | | Antibody (ARC0672) | | |
| ARX305 | CD70 | | | Skidmore et al., |
| | | | | 2023, Ann. Oncol. |
| | | | | 34 (Suppl. 2): S208. |
| ASD141 | CD11b | | | Chang et al., 2021, |
| | | | | J. Immunother. |
| | | | | Cancer 9 (Suppl. 2): |
| | | | | A1-A1054. |
| ASKB589 | CLDN18.2 | | | Zhang et al., 2023, |
| | | | | J. Clin. Oncol. 41 (4 |
| | | | | suppl.), 397. |
| ASP1951 | GITR | | | Seidel-Dugan et al., |
| | | | | 2018, J. |
| | | | | Immunother. Cancer |
| | | | | 6 (115). |
| AT002 | CD166 | available from multiple sources, including: | | |
| | | Recombinant Anti- | Antibodies.com, | |
| | | CD166 antibody | #A323924 | |
| | | Research Grade Anti- | AntibodySystem, | |
| | | Human CD166/ALCAM | #DHG64002 | |
| atibuclimab | CD14 | available from multiple sources, including: | | |
| | | Atibuclimab | MedChemExpress, | |
| | | | #HY-P99008 | |
| | | Atibuclimab (Anti- | SelleckChem, | |
| | | CD14) | #A2566 | |
| ATL301 | GD2 | Research Grade Anti- | AntibodySystem, | |
| | | Ganglioside GD2 | #DHK07801 | |
| | | (ATL301) | | |
| ATOR-1015 | OX40 | | | Veitonmaki et al., |
| | | | | 2018, Cancer Res. 78 |
| | | | | (13_Supplement): |
| | | | | 3623. |
| ATOR-1144 | GITR | | | Fritzell et al., 2019, |
| | | | | Cancer Res. 79 |
| | | | | (13_Supplement): |
| | | | | 4077. |
| axicabtagene | CD19 | | | Neelapu et al., |
| ciloleucel | | | | 2017, N. Engl. J. |
| | | | | Med. 377: 2531-44. |
| AZD0901 | CLDN18.2 | | | Raufi et al., 2024, J. |
| | | | | Clin. Oncol. 42 (16 |
| | | | | Suppl.), TPS3163. |
| AZD7789 | TIM-3 | | | Clancy-Thompson |
| | | | | et al., J. |
| | | | | Immunother. Cancer |
| | | | | 10 (Suppl. 2): A1- |
| | | | | A1603. |
| AZN-L50 | CD166 | available from multiple sources, including: | | |
| | | CD166, Human, mAB | Hycult Biotech, | |
| | | AZN-L50 | #HM2002 | |
| | | CD166 antibody [AZN- | GeneTex, | |
| | | L50] | #GTX18159 | |
| B4 | CD19 | | | Freedman et al., |
| | | | | 1987, Blood 70: |
| | | | | 418-427. |
| B4 HB12b | CD19 | | | Kansas & Tedder, |
| | | | | 1991, J. Immunol. |
| | | | | 147: 4094-4102; |
| | | | | Yazawa et al., 2005, |
| | | | | PNAS 102: 15178- |
| | | | | 15183; |
| | | | | Herbst et al., 2010, |
| | | | | J. Pharmacol. Exp. |
| | | | | Ther. 335: 213-222. |
| B7V3V2 | CD200 | Anti-Human CD200 | AntibodySystem, | |
| | | Antibody (B7V3V2) | #FHE30310 | |
| B43 | CD19 | | | Bejcek et al., 1995, |
| | | | | Cancer Res. 55 |
| | | | | (11): 2346-2351. |
| balstilimab | PD-1 | | | Moore et al., 2018, |
| | | | | J. Clin. Oncol. 36 |
| | | | | (15 suppl.): 3086. |
| barzolvolimab | CD117 | CDX 0159 | MedChemExpress, | |
| | | | #HY-P99462 | |
| basiliximab | CD25 | Basiliximab | ThermoFisher, | Onrust et al., 1999, |
| (STI-003) | | Recombinant Human | #MA5-41768 | Drugs 57, 207-213. |
| | | Monoclonal Antibody | | |
| BAT6026 | OX40 | | | Liang et al., 2023, |
| | | | | Front. Oncol., 13: |
| | | | | 1211759. |
| bavunalimab | LAG-3 | available from multiple sources, including: | | |
| | | Bavunalimab | MedChemExpress, | |
| | | | #HY-P99354 | |
| | | Bavunalimab | ChemScene, | |
| | | | #CS-0621488 | |
| BB2121 | BCMA | | | WO 2012/163805 |
| BC8-B10 | CD45 | | | Li et al., 2018, PLOS |
| | | | | One 13(10): |
| | | | | e0205135. |
| BCD-145 | CTLA-4 | available from multiple sources, including: | | |
| | | Nurulimab | MedChemExpress, | |
| | | | #HY-P99760 | |
| | | Nurulimab | AbMole, | |
| | | | #M24967 | |
| BCD-245 | GD2 | | | Clinical Trial ID: |
| | | | | NCT05782959 |
| belantamab | BCMA | | | U.S. pat. No. 9,273,141 |
| BGB-A445 | OX40 | | | Jiang et al., 2023, |
| | | | | Front. Med. 17(6): |
| | | | | 1170-1185. |
| bleselumab | CD40 | available from multiple sources, including: | | |
| | | Bleselumab | MedChemExpress, | |
| | | | #HY-P99257 | |
| | | Bleselumab | SelleckChem, | |
| | | | #A2634 | |
| BL-178-12C7 | CD45 | Anti-CD45 antibody | Abcam, | |
| | | | #ab243869 | |
| BLY3 | CD19 | | | Bejcek et al., 1995, |
| | | | | Cancer Res. 55 |
| | | | | (11): 2346-2351. |
| BMS-986156 | GITR | | | Siu et al., 2017, J. |
| | | | | Clin. Oncol. 35 |
| | | | | (15_suppl): 104. |
| BMS-986178 | OX40 | | | Gutierrez et al., |
| | | | | 2021, Clin. Cancer |
| | | | | Res. 27 (2): 460- |
| | | | | 472. |
| BMS-986179 | CD73 | | | Siu et al., 2018, |
| | | | | Cancer Res. |
| | | | | 78(13_Suppl): |
| | | | | CT180. |
| BMS-986218 | CTLA-4 | | | Engelhardt et al., |
| | | | | 2020, Cancer Res. 80 |
| | | | | (16_Supplement): |
| | | | | 4552. |
| BMS-986249 | CTLA-4 | | | Gutierrez et al., |
| | | | | 2020, J. Clin. Oncol. |
| | | | | 38 (15_suppl): 3058. |
| BMS-986258 | TIM-3 | | | Clinical Trial ID: |
| | | | | NCT03446040 |
| BMS-986393 | GPRC5D | | | Bal et al. (2023). |
| | | | | Blood 142 (Suppl. |
| | | | | 1): 219. |
| bococizumab | PCSK9 | available from multiple sources, including: | | |
| | | Bococizumab | MedChemExpress, | |
| | | | #HY-P99187 | |
| | | Bococizumab (Anti- | SelleckChem, | |
| | | PCSK9) | #A2788 | |
| | | Bococizumab | ThermoFisher, | |
| | | Recombinant Human | #MA5-42019 | |
| | | Monoclonal Antibody | | |
| bococizumab NEI | PCSK9 | | | Dyson et al. (2020). |
| | | | | MAbs 12: 1829335. |
| botensilimab | CTLA-4 | | | Wilky et al., 2022, J. |
| | | | | Immunother. |
| | | | | Cancer, 10 (Suppl. |
| | | | | 2): A1-A1603. |
| BP-02489 | CD10 | Accession No. NITE BP-02489 (JP) | | WO 2018/235247 |
| brexucabtagene | CD19 | | | Anderson et al., |
| autoleucel | | | | 2022, Ann. |
| | | | | Pharmacother. |
| | | | | 56(5): 609-619. |
| briquilimab | CD117 | available from multiple sources, including: | | |
| (JSP-191) | | Briquilimab | MedChemExpress, | |
| | | | #HY-P99488 | |
| | | Research Grade | Cell Sciences, | |
| | | Briquilimab | #DHC83102A | |
| BTI-322 | CD2 | Anti-Human CD2 | Creative Biolabs, | |
| | | Recombinant Antibody | #TAB-110CL | |
| | | (BTI-322) | | |
| BU12 | CD19 | | | Callard et al., 1992, |
| | | | | J. Immunology, |
| | | | | 148(10): 2983-2987 |
| Bu88 | CD8 | Anti-CD8 alpha | Abcam, | |
| | | Antibody | #ab316355 | |
| budigalimab | PD-1 | Budigalimab | MedChemExpress, | |
| | | | #HY-P99489 | |
| C2e10 | CD34 | available from multiple sources, including: | | |
| | | Mouse Anti-CD34 | Creative Biolabs, | |
| | | Recombinant Antibody | #HPAB-1044-FY- | |
| | | (clone C2e10); scFv | F(E) | |
| | | Fragment | | |
| | | Anti-CD34 [2E10], | Absolute | |
| | | Human IgG1, Kappa | Antibody, | |
| | | | #Ab01129 | |
| C5B12 | CD34 | available from multiple sources, including: | | |
| | | Human Anti-CD34 | Creative Biolabs, | |
| | | Recombinant Antibody | #HPAB-1043-FY- | |
| | | (clone C5B12); Fab | S(P) | |
| | | Fragment | | |
| | | Anti-CD34 [5B12], | Absolute | |
| | | Human IgG1, Kappa | Antibody, | |
| | | | #Ab00422 | |
| C8/144B | CD8α | available from multiple sources, including: | | |
| | | CD8 alpha Monoclonal | ThermoFisher, | |
| | | Antibody (C8/144B) | #MA5-13473 | |
| | | Anti-CD8 alpha | Abcam, | |
| | | antibody [C8/144B] | #ab17147 | |
| C11D5.3 | BCMA | | | WO 2010/104949 |
| C12A3.2 | | | | |
| C363.16A | CD45RB | CD45RB Monoclonal | ThermoFisher, | |
| | | Antibody (PE, | #12-0455-82 | |
| | | eBioscience ™) | | |
| cadonilimab | PD-1 | | | Pang et al., 2023, |
| | | | | MAbs 15(1): |
| | | | | 2180794. |
| camidanlumab | CD25 | | | Puzanov et al., |
| tesirine | | | | 2020, Ann. Oncol. |
| | | | | 31(suppl. 4), S710- |
| | | | | S711. |
| camrelizumab | PD-1 | Camrelizumab (anti- | SelleckChem, | |
| | | PD-1) | #A2016 | |
| carotuximab | CD105 | available from multiple sources, including: | | |
| | | Carotuximab | MedChemExpress, | |
| | | | #HY-P99494 | |
| | | Carotuximab (Anti- | SelleckChem, | |
| | | Endoglin/CD105) | #A2514 | |
| CBXS-1620 | SUSD2 | Rabbit Anti-SUSD2 | Creative Biolabs, | |
| | | Recombinant Antibody | #CBMAB-S4469- | |
| | | (CBXS-1620) | CQ | |
| CBXS-1671 | SUSD2 | Rabbit Anti-SUSD2 | Creative Biolabs, | |
| | | Recombinant Antibody | #CBMAB-S0094- | |
| | | (CBXS-1671) | CQ | |
| CBXS-1989 | SUSD2 | Mouse Anti-SUSD2 | Creative Biolabs, | |
| | | Recombinant Antibody | #CBMAB-S4758- | |
| | | (CBXS-1989) | CQ | |
| CBXS-1990 | SUSD2 | Mouse Anti-SUSD2 | Creative Biolabs, | |
| | | Recombinant Antibody | #CBMAB-S0156- | |
| | | (CBXS-1990) | CQ | |
| CBXS-3571 | SUSD2 | Human Anti-SUSD2 | Creative Biolabs, | |
| | | Recombinant Antibody | #CBMAB-S0386- | |
| | | (CBXS-3571) | CQ | |
| CBXS-3676 | SUSD2 | Rabbit Anti-SUSD2 | Creative Biolabs, | |
| | | Recombinant Antibody | #CBMAB-S0214- | |
| | | (CBXS-3676) | CQ | |
| CBYY-10413 | IL-12R | Mouse Anti-IL12RB2 | Creative Biolabs, | |
| | | Recombinant Antibody | #CBMAB-11506- | |
| | | (CBYY-10683) | YY | |
| C-CAR066 Leu16 | CD20 | | | Liang et al., 2021, J. |
| and 2.1.2 | | | | Clin. Oncol. 39(15) |
| | | | | suppl: 2508) |
| CD58 (LFA-3) | CD2 | Recombinant Human | R&D Systems, | |
| | | CD58/LFA-3 His-tag | #1689-CD | |
| | | Protein | | |
| CDLA68-1 | CD68 | CD68 Antibody [clone | NSJ Bioreagents, | |
| | | CDLA68-1] | #V7045 | |
| CDX-0158 | CD117 | Anti-SCFR/c-Kit/ | BiOrbyt, | |
| | | CD117 Reference | #orb1817461 | |
| | | Antibody (CDX-0158) | | |
| cedelizumab | CD4 | available from multiple sources, including: | | |
| | | Cedelizumab | MedChemExpress, | |
| | | | #HY-P99495 | |
| | | Cedelizumab (CD4) | ProSci, #10-093 | |
| cemiplimab | PD-1 | | | Burova et al., 2017, |
| | | | | Mol. Cancer Ther. |
| | | | | 16 (5), 861-870. |
| cetrelimab | PD-1 | Cetrelimab | MedChemExpress, | |
| | | | #HY-P99499 | |
| cetuximab | EGFR | available from multiple sources, including: | | |
| | | EGF R antibody \| C225 | BioRad, | |
| | | | #MCA6102 | |
| | | Cetuximab | MedChemExpress, | |
| | | | #HY-P9905 | |
| | | Cetuximab (anti-EGFR) | SelleckChem, | |
| | | | #A2000 | |
| cevostamab | CD3 | Cevostamab | MedChemExpress, | |
| | | | #HY-P99601 | |
| ch28/11 | SSEA-4 | Chimeric | Creative Biolabs, | |
| | | (Mouse/Human) Anti- | #PABJ-0121 | |
| | | SSEA-4 Recombinant | | |
| | | Antibody (clone | | |
| | | ch28/11) | | |
| cifurtilimab | CD40 | Cifurtilimab; SEA-CD40 | MedChemExpress, | |
| | | | #HY-P99603 | |
| ciltacabtagene | BCMA | | | Berdeja et al., 2021, |
| autoleucel | | | | Lancet 398 (10297): |
| | | | | 314-324. |
| CK6 | CD117 | | | Lebron et al., 2014, |
| | | | | Cancer Biol. Ther. |
| | | | | 15 (9): 1208-1218. |
| CL1659 | CD117 | CD117/c-kit Antibody | Novus | |
| | | (CL1657) | Biologicals, | |
| | | | #NBP2-52975 | |
| CLB-CD19 | CD19 | | | De Rie, 1989, Cell. |
| | | | | Immunol. 118:368- |
| | | | | 381 |
| clenoliximab | CD4 | Clenoliximab | Novus | |
| | | | Biologicals, | |
| | | | #NBP2-52681H | |
| Cmab-43 | CD133 | Recombinant Anti- | Abcam, | |
| | | CD133 antibody | #AB264538 | |
| CO.44B8 | CD43 | CD43 Monoclonal | ThermoFisher, | |
| | | Antibody (CO.44B8) | #MA5-28371 | |
| cobolimab | TIM-3 | Cobolimab | MedChemExpress, | |
| | | | #HY-P99827 | |
| CPP32 4-1-18 | Sca-1 | Caspase 3 Monoclonal | ThermoFisher, | |
| | | Antibody (CPP32 4-1-18) | MA1-16843 | |
| crefmirlimab | CD8α | available from multiple sources, including: | | Farwell et al., J Nucl |
| | | Crefmirlimab | MedChemExpress, | Med 63(5): 720-726 |
| | | | #HY-P99834 | (2021); |
| | | Crefmirlimab Biosimilar - | ProteGenix, #PX- | U.S. Pat. No. 10,414,820 |
| | | Anti-CD8A mAb - | TA1828-100 | |
| | | Research Grade | | |
| CS1002 | CTLA-4 | | | Bishnoi et al., 2021, |
| | | | | Ann. Oncol. 32, |
| | | | | (Suppl. 5): S840. |
| CT041 | CLDN18.2 | | | Qi et al., 2022, Nat. |
| | | | | Med. 28, 1189- |
| | | | | 1198. |
| CT103A | BCMA | | | U.S. Pat. No. 11,026,975 |
| (CAR0085) | | | | |
| cudarolimab | OX40 | Cudarolimab | MedChemExpress, | Kuang, Z et al. |
| | | | #HY-P99836 | Cancer Immunol |
| | | | | Immunother. 2020 |
| | | | | June; 69(6): 939-950. |
| cusatuzumab | CD70 | Cusatuzumab | MedChemExpress, | Riether, C et al. Nat |
| | | | #HY-P99014 | Med. 2020 |
| | | | | September; 26(9): 1459- |
| | | | | 1467. |
| CYT-91000 | CD25 | Research Grade Anti- | AntibodySystem, | |
| | | Human CD25/IL2RA | #DHB95805 | |
| | | (CYT-91000) | | |
| dacetuzumab | CD40 | Dacetuzumab | MedChemExpress, | Hussein, M et al. |
| | | | #HY-P99015 | Haematologica. |
| | | | | 2010 |
| | | | | May; 95(5): 845-8. |
| daclizumab | CD25 | Daclizumab | MedChemExpress, | Cohan, S. L. et al. |
| | | | #HY-108738 | Biomedicines. 2019 |
| | | | | Mar. 11; 7(1):18. |
| daratumumab | CD38 | Daratumumab (anti- | SelleckChem, | |
| | | CD38) | #A2027 | |
| davoceticept | CD28 | Davoceticept | MedChemExpress, | Patnaik, A. et al. |
| | | | #HY-P99844 | JCO 40, TPS2683- |
| | | | | TPS2683(2022). |
| DF-T1 | CD43 | CD43 Antibody (DF-T1) | Biotechnology, | |
| | | | Santa Cruz | |
| | | | #sc-6256 | |
| dinutuximab | GD2 | Dinutuximab | ThermoFisher, | Mohd, A. B. et al. J |
| | | | #MA5-42000 | Res Med Sci. 2023 |
| | | | | Sep 29;28:71. |
| dostarlimab | PD-1 | Dostarlimab | SelleckChem, | Mirza, M.R. et al. N |
| | | | #A2835 | Engl J Med. 2023 |
| | | | | Jun. 8; 388(23):2145- |
| | | | | 2158. |
| DS-7300A | B7H3 | | | Yamato et al., 2022, |
| | | | | Mol. Cancer Ther. |
| | | | | 21(4): 635-646. |
| EasySep ™ | | EasySep ™ Human T | Stemcell | |
| | | Cell Isolation Kit | Technologies, | |
| | | | #100-0695 | |
| efungumab | Stro-4 | Efungumab | MedChemExpress, | Karwa, R. et al. Ann |
| | | | #HY-P9962 | Pharmacother. 2009 |
| | | | | November: 43(11): 1818-23. |
| ektomab | GD2 | | | Ruf, P. et al. J |
| | | | | Transl Med. 2012 |
| | | | | Nov. 7; 10: 219. |
| ELISA MAX ™ | TNF-α | ELISA MAX ™ Deluxe | BioLegend, | |
| Deluxe Set | | Set Human TNF-α kit | #430204 | |
| Human TNF-α kit | | | | |
| elranatamab | CD3 | Research Grade | AntibodySystem, | Lesokhin, A. M. et |
| | | Elranatamab | #DHF92406 | al. Nat Med. 2023 |
| | | | | September; 29(9): 2259- |
| | | | | 2267 |
| EMB-02 | LAG-3 | | | Day et al., 2023, |
| | PD-1 | | | Ann. Oncol. 34 |
| | | | | (Suppl. 2): S625. |
| EMB-06 | BCMA | | | US 2023/0002489 |
| EMB-09 | OX40 | | | Li et al., 2023, Antib. |
| | PD-L1 | | | Ther. 6 (Suppl 1): |
| | | | | tbad014.008. |
| EMD 273063 | GD2 | Anti-GD2 (EMD | SelleckChem, | |
| | | 273063) | #A2916 | |
| encelimab | LAG-3 | Encelimab | MedChemExpress, | Perez-Santos, M. et |
| | | | #HY-P99922 | al. Expert Opin Drug |
| | | | | Discov. 2022 |
| | | | | December; 17(12): 1341- |
| | | | | 1355. |
| enoblituzumab | B7H3 | Anti-Human CD276 | Creative Biolabs, | |
| | | Recombinant Antibody | #TAB-424CQ | |
| EOS-448 | TIGIT | | | Cuende et al., 2022, |
| | | | | Cancer Res. 82 |
| | | | | (12_Supplement): |
| | | | | LB189. |
| EP322Y | CD45 | Anti-CD45 antibody | Abcam, | |
| | | [EP322Y] | #ab40763 | |
| EPR8913(2) | SUSD2 | Anti-SUSD2 antibody | Abcam, | |
| | | [EPR8913(2)] | #ab182147 | |
| EPR20545 | CD68 | Anti-CD68 antibody | Abcam, | |
| | | [EPR20545] | #ab213363 | |
| EPR26538-16 | CD8ß | Recombinant Anti-CD8 | Abcam, | |
| | | beta antibody | #ab300067 | |
| | | [EPR26538-16] | | |
| EPR26948-19 | FCRL5 | Anti-FCRL5 antibody | Abcam, | |
| | | [EPR26948-19] - BSA | #ab307569 | |
| | | and Azide free | | |
| | | (Detector) | | |
| EPR26948-67 | FCRL5 | Anti-FCRL5 antibody | Abcam, | |
| | | [EPR26948-67] - BSA | #ab307568 | |
| | | and Azide free | | |
| | | (Capture) | | |
| EPR27365-87 | | Anti-FCRL5 antibody | Abcam, | |
| | | [EPR27365-87] | #ab307730 | |
| epratuzumab | CD22 | Anti-Human CD22 | Creative Biolabs, | |
| | | Recombinant Antibody | #TAB-176 | |
| ET200-31 | FCRL5 | | | U.S. Pat. No. 10,913,796 |
| ET200-39 | | | | |
| ET200-69 | | | | |
| ET200-104 | | | | |
| ET200-105 | | | | |
| ET200-109 | | | | |
| ET200-117 | | | | |
| etigilimab | TIGIT | | | Mettu et al., 2022, |
| | | | | Clin. Cancer. Res. |
| | | | | 28(5): 882-892. |
| ezabenlimab | PD-1 | Ezabenlimab | MedChemExpress, | Zettl, M. et al. |
| | | | #HY-P99610 | Oncoimmunology. |
| | | | | 2022 Jun. |
| | | | | 16: 11(1): 2080328. |
| F10-89-4 | CD45 | Anti-CD45 antibody | Abcam, | |
| | | [F 10-89-4] - | #ab30470 | |
| | | Hematopoietic Stem | | |
| | | Cell Marker | | |
| F119 | FCRL5 | | | Ise et al. (2005) |
| | | | | Clin. Cancer Res. |
| | | | | 11(1): 87-96. |
| F25 | FCRL5 | Anti-FcRH5 Antibody, | Millipore Sigma, | |
| | | clone F25 | #MABF2103 | |
| F26 | FCRL5 | | | Franco et al. (2013) |
| | | | | J. Immunol. 190(11): |
| | | | | 5739-5746. |
| favezelimab | LAG-3 | Favezelimab | MedChemExpress, | Gareth, P. et al. |
| | | | #HY-P99613 | Cancer Res1 July |
| | | | | 2019; 79 |
| | | | | (13_Supplement): |
| | | | | CT106. |
| felzartamab | CD38 | Felzartamab | MedChemExpress, | |
| | | | #HY-P99616 | |
| FHVH33 | BCMA | | | WO 2019/006072 |
| fianlimab | LAG-3 | Fianlimab | MedChemExpress, | Omid, H. et al. JCO |
| | | | #HY-P99617 | 39, 9515-9515 |
| | | | | (2021). |
| FMC63 | CD19 | | | Nicholson et al., |
| | | | | 1997, Mol. Immun. |
| | | | | 34(16-17): 1157- |
| | | | | 1165; WO |
| | | | | 2018/213337, WO |
| | | | | 2015/187528 |
| forimtamig | GPRC5D | | | Harrison et al. |
| | | | | (2023). Clin. |
| | | | | Lymphoma |
| | | | | Myeloma Leuk. 23 |
| | | | | (Suppl. 2): S3-4. |
| FR4D11 | CD10 | Anti-Human CD10 | Stemcell | |
| | | Antibody, Clone | Technologies, | |
| | | FR4D11 | #60149 | |
| FR104CD | CD28 | Recombinant Anti- | Antibodies.com, | |
| | | CD28 Antibody | #A323936 | |
| | | [FR104] | | |
| FS118 | LAG-3 | | | Kraman et al., 2020, |
| | | | | Clin. Cancer Res. |
| | | | | 26 (13): 3333-3344. |
| FS120 | OX40 | | | Gaspar et al., 2020, |
| | CD137 | | | Cancer Immunol. |
| | | | | Res. 8 (6): 781-793. |
| G.813.2 | CD117 | Phospho-c-Kit (Tyr703) | ThermoFisher, | |
| | | Monoclonal Antibody | #MA5-14830 | |
| | | (G.813.2) | | |
| ganglidiximab | GD2 | | | Eger et al., 2016, |
| | | | | PLoS One 11 (3): |
| | | | | e0150479. |
| GEN-3014 | CD38 | | | Hiemstra et al., |
| | | | | 2023, EBioMedicine |
| | | | | 93: 104663. |
| geptanolimab | PD-1 | Geptanolimab | AntibodySystem, | Shi, Y. et al. Clin |
| | | | #DHH02224 | Cancer Res. 2020 |
| | | | | Dec. 15; 26(24): 6445- |
| | | | | 6452. doi: |
| | | | | 10.1158/1078- |
| | | | | 0432.CCR-20-2819. |
| | | | | Epub 2020 Oct. 12. |
| | | | | PMID: 33046518. |
| GIGA-564 | CTLA-4 | | | Stone, E. et al. |
| | | | | Journal for |
| | | | | Immuno Therapy of |
| | | | | Cancer, 2022; 10. |
| givastomig | CLDN18.2 | | | Gao et al., 2023, J. |
| | | | | Immunother. Cancer |
| | | | | 11(6): e006704. |
| GN1412 | CD28 | Theralizumab | MedChemExpress, | Hussain, K. et al. |
| | | | #HY-P9975 | Blood. 2015 Jan. |
| | | | | 1; 125(1): 102-10. |
| gresonitamab | CLDN18.2 | Gresonitamab | MedChemExpress, | |
| | | | #HY-P99350 | |
| grisnilimab | CD7 | Grisnilimab | MedChemExpress, | Scharnhorst, V, et |
| | | | #HY-P99650 | al. Gene. 2001 Aug. |
| | | | | 8; 273(2): 141-61. |
| | | | | doi: 10.1016/s0378- |
| | | | | 1119(01)00593-5. |
| | | | | PMID: 11595161. |
| GSK2831781 | LAG-3 | | | Clinical Trial ID: |
| | | | | NCT03893565 |
| GSK3174998 | OX40 | | | Infante, J. R. et al. |
| | | | | JCO 34, TPS3107- |
| | | | | TPS3107 (2016). |
| GWN323 | GITR | | | Piha-Paul, S. A. et |
| | | | | al. Journal for |
| | | | | Immuno Therapy of |
| | | | | Cancer |
| | | | | 2021; 9: e002863. |
| h4C8 | CD34 | Anti-Human CD34 | Creative Biolabs, | |
| | | Recombinant Antibody | #TAB-149LC- | |
| | | Fab Fragment (h4C8) | F(E) | |
| H22 | CD64 | | | Hristodorov, D. et al. |
| | | | | MAbs. |
| | | | | 2014; 6(5): 1283-9. |
| | | | | doi: |
| | | | | 10.4161/mabs.32182. |
| | | | | PMID: 25517313; |
| | | | | PMCID: PMC4622438. |
| H44 | IL-18Rα | APC anti-human | Biolegend, | |
| | | CD218a (IL-18Rα) | #313814 | |
| | | Antibody | | |
| H65 | CD5 | CD5 Antibody | Novus | |
| | | | Biologicals, | |
| | | | #NB100-2667 | |
| hB-F5 | CD4 | NeuroMab ™ Anti-CD4 | Creative Biolabs, | |
| | | BBB Shuttle Antibody, | #NRZP-1022- | |
| | | Clone hB-F5 | ZP3717 | |
| HBM1007 | CD73 | | | Gan et al., 2020, |
| | | | | 2020 AACR Poster |
| | | | | #6056. |
| HBM4003 | CTLA-4 | | | Shanzhi, G. et al. |
| | | | | JCO 40, 2641-2641 |
| | | | | (2022). |
| HC34LC14 | CD117 | c-Kit Recombinant | ThermoFisher, | |
| | | Rabbit Monoclonal | #701494 | |
| | | Antibody (HC34LC14) | | |
| HCD27.15 | CD27 | | | U.S. Pat. No. 9,527,916 |
| HD37 | CD19 | | | Pezutto et al., 1987, |
| | | | | J. Immunol. 138(9): |
| | | | | 2793-2799 |
| HE3 | CD5 | | | U.S. Pat. No. 5,770,196 |
| HFB301001 | OX40 | | | Fulton, R. et al. |
| | | | | Cancer Res 1 July |
| | | | | 2021; 81 |
| | | | | (13_Supplement): |
| | | | | 1882. |
| HI30 | CD45 | Purified anti-human | BioLegend, | |
| | | CD45 Antibody | #304001 | |
| HIT8a | CD8α | Purified anti-human | BioLegend, | |
| | | CD8a Antibody | #300901 | |
| HLX26 | LAG-3 | | | Rujiao, L. et al. JCO |
| | | | | 41, e14671-e14671 |
| | | | | (2023). |
| HLX51 | OX40 | | | Clinical Trial ID: |
| | | | | NCT05788107 |
| HM48-1 | CD48 | CD48 Monoclonal | ThermoFisher, | |
| | | Antibody (HM48-1), | #17-0481-82 | |
| | | APC, eBioscience ™ | | |
| HPAB-0260-YJ | CD200 | Human Anti-CD200 | Creative Biolabs, | |
| | | Recombinant Antibody | #HPAB-0260-YJ- | |
| | | | F(E) | |
| HPAB-3334LY | PODXL | Human Anti-PODXL | Creative Biolabs, | |
| | | Recombinant Antibody | #HPAB-3334LY | |
| HPAB-MO612-YC | PODXL | Human Anti-LAM | Creative Biolabs, | |
| | | Recombinant Antibody | #HPAB-M0561- | |
| | | | YC | |
| HPN217 | BCMA | | | U.S. Pat. No. 11,136,403 |
| Hu001-MMAE | CD73 | | | Jin et al., 2020, Mol. |
| | | | | Cancer Ther. 19 |
| | | | | (11): 2340-2352. |
| hu3G8-5.1-N297Q | CD16a | | | U.S. Pat No. 7,351,803 |
| hu14.18k322A | GD2 | Anti-Human GD2 | Creative Biolabs, | |
| | | Recombinant Antibody | #TAB-304CL | |
| | | (hu14.18K322A) | | |
| Hu222 | OX40 | | | U.S. Pat No. 9,006,399 |
| huAA98 | CD146 | Anti-Human MCAM | Creative Biolabs, | |
| | | Recombinant Antibody | #TAB-444MZ | |
| | | (huAA98) | | |
| Huly-m2 | CD7 | CD7 (T-Cell Leukemia | \|ThermoFisher, | |
| | | Marker) Monoclonal | #924-MSM2- | |
| | | Antibody (HuLy-m2) | P1ABX | |
| HuMab 611 | CD64 | | | WO 2006/002438 |
| Human IgG1 poly- | lgG1 | Human IgG1 Poly- | Medna Scientific, | |
| specificity control | | Specificity Control | #H1308 | |
| antibody | | Antibody | | |
| Human IgG4 | lgG4 | Human IgG4 Isotype | Medna Scientific, | |
| isotype control | | Control Antibody | #H1314 | |
| antibody | | | | |
| humanized AT-10 | CD32A | CD32 Antibody | Biorad, | |
| | | | #MCA1075 | |
| humanized IV.3 | CD32A | Anti-Human CD32 | StemCell | |
| | | Antibody, Clone IV.3 | Technologies, | |
| | | | #60012 | |
| HuMax-TAC | CD25 | Camidanlumab | MedChemExpress, | |
| | | | #HY-P99233 | |
| huRH105 | CD105 | Anti-CD105 [huRH105- | Absolute | |
| | | 1] | Antibody, | |
| | | | #Ab04017 | |
| IAB22M | CD8 | | | U.S. Pat No. 11,254,744 |
| IAb_CysDb3b_CD8 | CD8 | | | U.S. Pat No. 10,414,820 |
| IAb_M1b_CD8 | CD8 | | | |
| IAb_M2b_CD8 | CD8 | | | |
| ibalizumab | CD4 | Ibalizumab | MedChemExpress, | |
| | | | #HY-P99028 | |
| IBI110 | LAG-3 | | | Xu et al., 2022, J. |
| | | | | Clin. Oncol. 40 |
| | | | | (16_suppl): 2650. |
| IBI310 | CTLA-4 | | | Zhou et al., 2022, J. |
| | | | | Clin. Oncol. 40 |
| | | | | (4_suppl): 421. |
| IBI318 | PD-1 | Reozalimab | MedChemExpress, | |
| | | (LY3434172) | #HY-P9996 | |
| IBI323 | LAG-3 | | | Ni et al., 2020, |
| | | | | Cancer Res. 80 |
| | | | | (16_Supplement): |
| | | | | 3270. |
| IBI325 | CD73 | | | Zhou et al., 2023, |
| | | | | Int. J. Biol. |
| | | | | Macromol. 229:158- |
| | | | | 167. |
| IBI343 | CLDN18.2 | | | Yu et al., 2024, J. |
| | | | | Clin. Oncol. 42 (16 |
| | | | | Suppl.), 3037. |
| ibritumomab | CD20 | Ibritumomab Biosimilar - | ProteoGenix, | |
| | | Anti- | #PX-TA1620 | |
| | | MS4A1(CD20,MS4A-1) | | |
| | | mAb - Research Grade | | |
| idecabtagene | BCMA | | | Munshi et al., 2021, |
| vicleucel | | | | N. Engl. J. Med. |
| | | | | 384: 705-716. |
| ieramilimab | LAG-3 | Ieramilimab | MedChemExpress, | Schoffski, P et al. J |
| | | | #HY-P99027 | Immunother Cancer. |
| | | | | 2022 |
| | | | | February; 10(2): e003776. |
| IL-A116 | CD45RO | CD45RO Monoclonal | ThermoFisher, | |
| | | Antibody (IL-A116) | #MA5-28403 | |
| imaprelimab | CD146 | Imaprelimab | MedChemExpress, | Pelletier, J. P. R. Et |
| | | | #HY-P99658 | al. Immunologic |
| | | | | Concepts in |
| | | | | Transfusion |
| | | | | Medicine, Elsevier, |
| | | | | 2020, 251-348. |
| INBRX-106 | OX40 | | | Clinical Trial ID: |
| | | | | NCT04198766 |
| INCA00186 | CD73 | | | Stewart, S. et al. |
| | | | | Cancer Res 1 July |
| | | | | 2021; 81 |
| | | | | (13_Supplement): |
| | | | | LB174. |
| INCA32459 | LAG-3 | | | Clinical Trial ID: |
| | | | | NCT05577182 |
| INCAGN1949 | OX40 | | | Davis, E. J. et al. J |
| | | | | Immunother Cancer. |
| | | | | 2022 |
| | | | | October; 10(10): e004235. |
| | | | | doi: 10.1136/jitc- |
| | | | | 2021-004235. |
| | | | | PMID: 36316061; |
| | | | | PMCID: PMC9628691. |
| INCAGN2385 | LAG-3 | | | Clinical Trial ID: |
| | | | | NCT03538028 |
| INCAGN02390 | TIM-3 | | | Clinical Trial ID: |
| | | | | NCT03652077 |
| inezetamab | CD4 | Inezetamab | MedChemExpress, | |
| | | | #HY-P99663 | |
| inolimomab | CD25 | Research Grade | Antibody System, | |
| | | Inolimomab | #DHB95804 | |
| inotuzumab | CD22 | Humanized Anti-CD22 | Creative Biolabs, | |
| | | Recombinant Antibody | #TAB-198 | |
| | | (clone Inotuzumab) | | |
| IPH5301 | CD73 | | | Gonçalves et al., |
| | | | | 2022, Immuno- |
| | | | | Oncology and |
| | | | | Technology 16 (S1): |
| | | | | 199TiP. |
| ipilimumab | CTLA-4 | Ipilimumab | MedChemExpress, | |
| | | | #HY-P9901 | |
| IPO-3 | CD150 | Anti-SLAM/CD150 | Abcam, #ab2604 | |
| | | antibody [IPO-3] | | |
| isatuximab | CD38 | Isatuximab (anti-CD38) | SelleckChem, | |
| | | | #A2039 | |
| iscalimab | CD40 | Iscalimab | MedChemExpress, | |
| | | | #HY-P99670 | |
| ispectamab | BCMA | | | US 2021/0130483 |
| IT1208 | CD4 | | | Shitara, K et al. J |
| | | | | Immunother Cancer. |
| | | | | 2019 Jul. |
| | | | | 24; 7(1): 195. |
| ivuxolimab | OX40 | Ivuxolimab | MedChemExpress | Diab, A et al. Clin |
| | | | (MCE), #HY- | Cancer Res. 2022 |
| | | | P99159 | Jan. 1; 28(1): 71-83. |
| JAB-BX102 | CD73 | | | Clinical Trial ID: |
| | | | | NCT05174585 |
| JK08 | CTLA-4 | | | Kotecki, et al., 2023, |
| | | | | Ann. Oncol. 34(S2), |
| | | | | pg. S635. |
| JM1-24-3 | CD146 | | | Feng et al., 2020, J. |
| | | | | Exp. Clin. Cancer |
| | | | | Res. 39: 273. |
| JM7A4 | IL-15Rα | available from multiple sources, including: | | |
| | | Anti-IL-15RA antibody | Abcam, | |
| | | [JM7A4] | #ab91270 | |
| | | IL-15R alpha Antibody | Novus | |
| | | (JM7A4) - BSA Free | Biologicals, | |
| | | | #NBP1-43238 | |
| JMW-3B3 | CTLA-4 | Afuco ™ Anti-Human | Creative Biolabs, | |
| | | CTLA4 ADCC | #AFC-185CL | |
| | | Recombinant Antibody | | |
| | | (JMW-3B3), ADCC | | |
| | | Enhanced | | |
| JNJ-64164711 | GITR | Research Grade Anti- | \|#DHJ89808 | Holland et al., 2018, |
| | | Human CD357/TNFRSF18/GITR | AntibodySystem, | Cancer Res. 78 |
| | | | | (13_Supplement): |
| | | | | 3813. |
| JS007 | CTLA-4 | | | Guan et al., 2023, |
| | | | | MAbs 15(1): |
| | | | | 2153409. |
| JTX-2011 | ICOS | Vopratelimab | MedChemExpress, | |
| | | | #HY-P99382 | |
| K45 | CD117 | c-Kit Monoclonal | ThermoFisher, | |
| | | Antibody (K45) | #MA5-12944 | |
| K117 | CD90 | Anti-CD90 Antibody, | RayBiotech, | |
| | | Purified | #136-00048 | |
| KD6001 | CTLA-4 | | | Chen et al., 2024, |
| | | | | Cancer Res. 84 |
| | | | | (6_Suppl.): 1350. |
| keliximab | CD4 | available from multiple sources, including: | | |
| | | Keliximab | MedChemExpress, | |
| | | | #HY-P99680 | |
| | | Keliximab (CD4) - | ProSci, #10-178 | |
| | | Research Grade | | |
| | | Biosimilar | | |
| Ki-M7 | CD68 | available from multiple sources, including: | | |
| | | CD68 antibody \| Ki-M7 | Bio-Rad, | |
| | | | #MCA2375F | |
| | | CD68 Monoclonal | ThermoFisher, | |
| | | Antibody (Ki-M7), FITC | #MA1-82715 | |
| kit2c75 | CD117 | CD117 (c-Kit) | ThermoFisher, | |
| | | Monoclonal Antibody | #MA5-28805 | |
| | | (kit2c75), PE | | |
| KM666 | GD2 | | | Nakamura et al., |
| | | | | 2001, Cancer |
| | | | | Immunol. |
| | | | | Immunother. 50, |
| | | | | 275-284. |
| KN044 | CTLA-4 | | | Clinical Trial ID: |
| | | | | NCT04126590 |
| L17F12 | CD5 | available from multiple sources, including: | | |
| | | Purified anti-human | BioLegend, | |
| | | CD5 Antibody | #364001 | |
| | | CD5 Antibody | Santa Cruz | |
| | | (L17F12) | Biotechnology, | |
| | | | #sc-18898 | |
| LAMP4-824 | CD68 | available from multiple sources, including: | | |
| | | CD68 Monoclonal | Biotium, #0824 | |
| | | Mouse Antibody | | |
| | | (LAMP4/824) | | |
| | | CD68 Monoclonal | ThermoFisher, | |
| | | Antibody (LAMP4, 824) | #968-MSM4-P1 | |
| LB1410 | TIM-3 | | | Liu, 2023, J. Clin. |
| | | | | Oncol. 41 |
| | | | | (16_suppl): |
| | | | | TPS2663. |
| LCAR-B38M | BCMA | | | Zhao et al., 2018, J. |
| | | | | Hematol. Oncol. |
| | | | | 11(1): 141; |
| | | | | WO 2018/028647 |
| LCAR-C18S | CLDN18.2 | | | Zhen et al., 2023, J. |
| (LB1908) | | | | Clin. Oncol. 41 (4 |
| | | | | suppl.), TPS480. |
| Leu16 | CD20 | | | Wu et al., 2001, |
| | | | | Protein Engineering. |
| | | | | 14(12): 1025-1033 |
| linvoseltamab | BCMA | | | U.S. Pat No. 11,919,965 |
| lisocabtagene | CD19 | | | Abramson et al., |
| maraleucel | | | | 2020, Lancet 396: |
| | | | | 839-852. |
| LM-302 | CLDN18.2 | | | Huang et al., 2022, |
| | | | | Eur. J. Cancer 174 |
| | | | | (Suppl. 1): S41-S42. |
| LOP628 | CD117 | Anti-cKit-maitansine | Creative Biolabs, | |
| | | ADC (LOP628) | #ADC-L039 | |
| lorigerlimab | PD-1 | | | Luke et al., J. Clin. |
| | | | | Oncol. 41 (6_suppl): |
| | | | | 155. |
| lorvotuzumab | CD56 | available from multiple sources, including: | | |
| | | Lorvotuzumab | MedChemExpress, | |
| | | | #HY-P99372 | |
| | | Lorvotuzumab | ThermoFisher, | |
| | | Recombinant Human | #MA5-41708 | |
| | | Monoclonal Antibody | | |
| LT-2 | CD2 | CD2 Monoclonal | ThermoFisher, | |
| | | Antibody (LT2) | #MA1-10131 | |
| LT-8 | CD8 | available from multiple sources, including: | | |
| | | Mouse monoclonal | Antibodies.com, | |
| | | [LT8] antibody to CD8 | #A242891 | |
| | | (PE) | | |
| | | Monoclonal Anti-CD8 | Millipore Sigma, | |
| | | antibody | #SAB4701064 | |
| lucatumumab | CD40 | available from multiple sources, including: | | |
| | | Lucatumumab | MedChemExpress, | |
| | | | #HY-P99167 | |
| | | Anti-CD40 | Creative Biolabs, | |
| | | Recombinant Antibody | #TAB-759 | |
| | | (Lucatumumab) | | |
| lulizumab | CD28 | available from multiple sources, including: | | |
| | | Lulizumab | ThermoFisher, | |
| | | Recombinant Human | #MA5-42032 | |
| | | Monoclonal Antibody | | |
| | | Humanized Anti-CD28 | Creative Biolabs, | |
| | | Recombinant Antibody | #TAB-H46 | |
| | | (clone Lulizumab) | | |
| LVGN6051 | CD137 | | | Qi et al., 2019, Nat. |
| | | | | Commun. 10(1): |
| | | | | 2141. |
| LY75_A1 | CD205 | LY75/DEC-205 Protein | Antibodies | |
| | | (AA 28-1667) (His tag) | Online, | |
| | | | #ABIN7275222 | |
| LY3321367 | TIM-3 | | | Harding et al., 2021, |
| | | | | Clin. Cancer Res. |
| | | | | 27(8): 2168-2178. |
| LY3415244 | TIM-3 | | | Hellmann et al., |
| | | | | 2021, Clin. Cancer |
| | | | | Res. 27(10): 2773- |
| | | | | 2781. |
| M108 | CLDN18.2 | | | Jin et al., 2024, J. |
| (FG-M108) | | | | Clin. Oncol. 42 (16 |
| | | | | suppl.), 4142. |
| M971 | CD22 | Anti-Siglec-2/CD22 | SelleckChem, | |
| | | (NCI m971) | #A3057 | |
| mAb19 | CD73 | | | Wurm et al., 2021, |
| | | | | Mol. Cancer Ther. |
| | | | | 20(11): 2250-2261 |
| MAB107 | CD11b | available from multiple sources, including: | | |
| | | Anti-CD11b/CD18 | Millipore Sigma, | |
| | | Antibody, clone | #MABF2085 | |
| | | mAb107 | | |
| | | Anti-human IgE mAb | Mabtech, #3810- | |
| | | (107), unconjugated | 3 | |
| MAB1472-100 | IL-15Rα | available from multiple sources, including: | | |
| | | Human IL-15R alpha | R&D Systems, | |
| | | Antibody | #MAB1472 | |
| | | IL-15R alpha Antibody | Novus | |
| | | (1020456) | Biologicals, | |
| | | [Unconjugated] | #MAB1472 | |
| MAB5511 | IL-15Rα | Mouse IL-15R alpha | R&D Systems, | |
| | | Antibody | #MAB5511 | |
| MAI1738 | PODXL | Research Grade Anti- | AntibodySystem, | |
| | | Human PODXL | #DHA 16801 | |
| | | (MAI1738) | | |
| MAT304 | CD5 | Research Grade Anti- | AntibodySystem, | |
| | | Human CD5 (MAT 304) | #DHC18804 | |
| MAX.16H5 | CD4 | | | Guse et al., 1994, J. |
| | | | | Chromatogr. A 661: |
| | | | | 13-23. |
| MB-106 | CD20 | Fred Hutchinson Cancer Research Center | | Shadman et al., |
| | | | | 2019, Blood |
| | | | | 134(Suppl.1): 3235 |
| MC631 | SSEA-3 | available from multiple sources, including: | | |
| | | Anti-SSEA3 antibody | Abcam, | |
| | | [MC631] | #ab16286 | |
| | | Anti-Mouse SSEA-3 | StemCell | |
| | | Antibody, Clone MC- | Technologies, | |
| | | 631 | #60061.1 | |
| MC-813-70 | SSEA-4 | available from multiple sources, including: | | |
| | | SSEA4 Monoclonal | ThermoFisher, | |
| | | Antibody (MC-813-70) | #MA1-021 | |
| | | Anti-Human SSEA-4 | StemCell | |
| | | Antibody, Clone MC- | Technologies, | |
| | | 813-70 | #60062 | |
| MCARH109 | GPRC5D | | | Mailankody et al., N |
| | | | | Engl J Med. |
| | | | | 387(13): 1196-1206 |
| | | | | (2022) |
| MDE-8 | CD32A | Anti-CD32 [MDE-8] | Absolute | |
| | | | Antibodies, | |
| | | | #Ab02072 | |
| MDX-1203 | CD70 | | | Owonikoko et al., |
| | | | | 2014, J. Clin. Oncol. |
| | | | | 32: 5s, (suppl; abstr |
| | | | | 2558). |
| MDX-1411 | CD70 | Anti-CD70 monoclonal | Creative Biolabs, | |
| | | antibody (MDX-1411) | #Gly-008CL | |
| MEDI0680 | PD-1 | | | Naing et al., 2019, J. |
| | | | | Immunother. Cancer |
| | | | | 7: 225. |
| MEDI1873 | GITR | | | Tigue et al., 2017, |
| | | | | Oncoimmunology |
| | | | | 6(3): e1280645. |
| MEDI2228 | BCMA | | | U.S. Pat No. 10,988,546 |
| MEDI5752 | PD-1 | | | Dovedi et al., 2021, |
| | | | | Cancer Discov. |
| | | | | 11(5): 1100-1117. |
| MEDI6383 | OX40 | | | Hammond et al., |
| | | | | 2015, J. Clin. Oncol. |
| | | | | 33 (15_suppl): 3056. |
| MEDI6469 | OX40 | | | Leidner et al., 2015, |
| | | | | J. Clin. Oncol. 33 |
| | | | | (15_suppl): TPS6083. |
| MEM-28 | CD45 | Anti-CD45 antibody | Abcam, #ab8216 | |
| | | [MEM-28] | | |
| MEM-31 | CD8 | available from multiple sources, including: | | |
| | | CD8 Monoclonal | ThermoFisher, | |
| | | Antibody (MEM-31) | #MA1-19082 | |
| | | Monoclonal Anti-CD8 | SigmaAldrich, | |
| | | antibody produced in | #SAB4700084 | |
| | | mouse | | |
| MEM-55 | CD45RB | Anti-CD45RB antibody | Abcam, #ab8218 | |
| | | [MEM-55] | | |
| MEM-59 | CD43 | CD43 Monoclonal | ThermoFisher, | |
| | | Antibody (MEM-59) | #MA1-19009 | |
| MEM-65 | CD2 | available from multiple sources, including: | | |
| | | CD2 Monoclonal | ThermoFisher, | |
| | | Antibody (MEM-65) | #MA1-19070 | |
| | | Anti-CD2 Antibody | Antibodies.com, | |
| | | [MEM-65] | #A86387 | |
| MEM-87 | CD8 | available from multiple sources, including: | | |
| | | Anti-CD8 Antibody | Antibodies.com, | |
| | | (MEM-87) | #A85681 | |
| | | Monoclonal Anti-CD8 | SigmaAldrich, | |
| | | antibody produced in | #SAB4700089 | |
| | | mouse | | |
| MEM-102 | CD48 | CD48 Monoclonal | ThermoFisher, | |
| | | Antibody (MEM-102) | #MA1-19119 | |
| mezagitamab | CD38 | Mezagitamab | MedChemExpress, | |
| | | | #HY-P99730 | |
| MGC018 | B7H3 | | | Scribner et al., |
| | | | | 2020, Mol. Cancer |
| | | | | Ther. 19(11): 2235- |
| | | | | 2244. |
| MGTA-117 | CD117 | | | Westervelt et al., |
| | | | | 2022, Blood 140 |
| | | | | (Supplement 1): |
| | | | | 2117-2119. |
| MiK-Beta-1 | CD122 | CD122 Monoclonal | ThermoFisher, | |
| | | Antibody (MIK-Beta 1) | #MA1-35896 | |
| miptenalimab | LAG-3 | Miptenalimab | MedChemExpress, | |
| | | | #HY-P99736 | |
| mitazalimab | CD40 | | | Calvo et al., 2019, J. |
| | | | | Clin. Oncol. 37 |
| | | | | (15_suppl): 2527. |
| MK-1248 | GITR | | | Geva et al., 2020, |
| | | | | Cancer 126(22): |
| | | | | 4926-4935. |
| MK-4166 | GITR | | | Sukumar et al., |
| | | | | 2017, Cancer Res. |
| | | | | 77 (16): 4378-4388. |
| MORAb-028 | GD2 | MORAb-028 | ThermoFisher, | |
| | | Recombinant Human | #MA5-41810 | |
| | | Monoclonal Antibody | | |
| moxetumomab | CD22 | Anti-CD22 | Creative Biolabs, | |
| | | (Moxetumomab | #ADC-W-800 | |
| | | pasudotox)-SMCC- | | |
| | | DM1 ADC | | |
| mShad150 | CD150 | CD150 Monoclonal | ThermoFisher, | |
| | | Antibody (mShad150), | #12-1502-82 | |
| | | PE, eBioscience ™ | | |
| M-T413 | CD4 | | | Rieber et al., 1992, |
| | | | | PNAS 89(22): |
| | | | | 10792-6. |
| MT95-4 | CD13 | | | WO 2021/072312 |
| MRTX1011A | CD4 | | | Scheerens et al., |
| | | | | 2011, Arthritis Res. |
| | | | | Ther. 13 (5): R177. |
| MT807-R1 | CD8α | Anti-CD8 alpha | NIH Nonhuman | Nakamura-Hoshi et |
| | | (MT807R1) | Primate Reagent | al., 2024, Mol. Ther. |
| | | | Resources, | 32 (7): 1-12. |
| | | | #AB_2716320 | |
| mupadolimab | CD73 | Mupadolimab | MedChemExpress, | |
| | | | #HY-P99181 | |
| My10 | CD34 | Anti-CD34 antibody | Abcam, | |
| | | [My10] | #ab245689 | |
| naxitamab | GD2 | Naxitamab | MedChemExpress, | |
| | | | #HY-P99206 | |
| Nbl57 | CD13 | | | WO 2021/072312 |
| ND-742 | SSEA-3 | Anti-SSEA-3 | Creative | |
| | | monoclonal antibody | Diagnostics, | |
| | | | #DMAB12907 | |
| nivatrotamab | GD2 | Nivatrotamab | MedChemExpress, | |
| | | | #HY-P99757 | |
| nivolumab | PD-1 | available from multiple sources, including: | | |
| | | Nivolumab (anti-PD-1) | SelleckChem, | |
| | | | #A2002 | |
| | | Nivolumab | Evidentic, | |
| | | | #8006657 | |
| NN2101 | CD117 | | | Kim et al., 2020, Int. |
| | | | | J. Biol. Macromol. |
| | | | | 159: 66-78. |
| nofazinlimab | PD-1 | Nofazinlimab | MedChemExpress, | |
| | | | #HY-P99758 | |
| nurulimab | CTLA-4 | available from multiple sources, including: | | |
| | | Nurulimab | MedChemExpress, | |
| | | | #HY-P99760 | |
| | | Nurulimab (Anti-CTLA- | SelleckChem, | |
| | | 4/CD152 | #A2859 | |
| NZV930 | CD73 | | | Fu et al., 2022, |
| | | | | Cancer Res. |
| | | | | 82(12_Suppl), |
| | | | | CT503. |
| obinutuzumab | CD20 | available from multiple sources, including: | | |
| | | Obinutuzumab | MedChemExpress, | |
| | | | #HY-P9910 | |
| | | Obinutuzumab (anti- | SelleckChem, | |
| | | CD20) | #A2023 | |
| | | Obinutuzumab | ThermoFisher, | |
| | | Monoclonal Antibody | #A01946-40 | |
| | | (16B7) | | |
| ocrelizumab | CD20 | available from multiple sources, including: | | |
| | | Ocrelizumab | MedChemExpress, | |
| | | | #HY-P9960 | |
| | | Ocrelizumab (Anti- | SelleckChem, | |
| | | CD20) | #A2561 | |
| | | Ocrelizumab | ThermoFisher, | |
| | | Recombinant Human | #MA5-41829 | |
| | | Monoclonal Antibody | | |
| odronextamab | CD20 | available from multiple sources, including: | | |
| | CD3 | Odronextamab | MedChemExpress, | |
| | | | #HY-P99038 | |
| | | Odronextamab | ThermoFisher, | |
| | | Recombinant Human | #MA5-42283 | |
| | | Monoclonal Antibody | | |
| ofatumumab | CD20 | available from multiple sources, including: | | |
| | | Ofatumumab | MedChemExpress, | |
| | | | #HY-P9961 | |
| | | Ofatumumab Biosimilar - | ProteoGenix, | |
| | | Anti-MS4A1, CD20 | #PX-TA1146 | |
| | | mAb - Research | | |
| | | Grade | | |
| | | Ofatumumab | ThermoFisher, | |
| | | Recombinant Human | #MA5-41813 | |
| | | Monoclonal Antibody | | |
| OKT1 | CD5 | CD5 Unconjugated | Biohippo, | |
| | | OKT1 IgG1k (RUO) | #BHA19900487 | |
| OKT3 | CD3 | CD3 Monoclonal | ThermoFisher, | |
| (muromonab- | | Antibody (OKT3), | #14-0037-82 | |
| CD3) | | eBioscience ™ | | |
| OKT4 | CD4 | Brilliant Violet 650 ™ | BioLegend, | |
| | | anti-human CD4 | #317436 | |
| | | Antibody | | |
| OKT8 | CD8α | CD8α Monoclonal | ThermoFisher, | |
| | | Antibody (OKT8) | #14-0086-80 | |
| OKT11 | CD2 | available from multiple sources, including: | | U.S. Pat No. 4,364,937 |
| | | OKT11 | ATCC, #CRL- | |
| | | | 8027 | |
| | | | MilliporeSigma, | |
| | | | #86050802 | |
| oleclumab | CD73 | Oleclumab | MedChemExpress, | |
| | | | #HY-P99039 | |
| ONC-392 | CTLA-4 | | | Rolfo et al., 2020, J. |
| | | | | Clin. Oncol. 38 |
| | | | | (15_suppl): |
| | | | | TPS3159. |
| OriCAR-017 | GPRC5D | | | Rodriguez-Otero et |
| | | | | al., Blood Cancer J. |
| | | | | 14(1): 24 (2024) |
| OS2966 | CD29 | | | Nigim et al., 2019, |
| | | | | Target. Oncol. 14: |
| | | | | 479-489. |
| osemitamab | CLDN18.2 | | | Qian et al., 2023, |
| | | | | Ann. Oncol. 34 |
| | | | | (Suppl. 2), S873, |
| | | | | 1560P. |
| otelixizumab | CD3 | Otelixizumab | MedChemExpress, | |
| | | | #HY-P99211 | |
| OTI14B1 | CD117 | KIT Mouse anti- | Fisher Scientific, | |
| | | Human, Clone: | #50-166-9129 | |
| | | OTI14B1, lyophilized, | | |
| | | TrueMAB ™ | | |
| OTI1B6 | | KIT Monoclonal | ThermoFisher, | |
| | | Antibody (OTI1B6), | #CF808721 | |
| | | TrueMAB ™ | | |
| OTI1E2 | | CD117 Mouse | OriGene, | |
| | | Monoclonal Antibody | #TA801037 | |
| | | [Clone ID: OTI1E2] | | |
| OTI1F6 | | KIT Monoclonal | ThermoFisher, | |
| | | Antibody (OTI1F6), | #CF801131 | |
| | | TrueMAB ™ | | |
| OTI2B12 | | CD117 Mouse | OriGene, | |
| | | Monoclonal Antibody | #CF808739 | |
| | | [Clone ID: OTI2B12] | | |
| OTI2B5 | | KIT Monoclonal | ThermoFisher, | |
| | | Antibody (OTI2B5), | #TA801047 | |
| | | TrueMAB ™ | | |
| OTI2C1D5 | | KIT Monoclonal | ThermoFisher, | |
| | | Antibody (OTI2C1D5), | #CF801043 | |
| | | TrueMAB ™ | | |
| OTI2C1H4 | | KIT Monoclonal | ThermoFisher, | |
| | | Antibody (OTI2C1H4), | #CF805183 | |
| | | TrueMAB ™ | | |
| OTI2E3 | | Monoclonal Mouse | LSBio, | |
| | | anti-Human c-Kit/ | #LS-C789071- | |
| | | CD117 Antibody | 100 | |
| | | (Carrier-free, | | |
| | | aa546-976, IHC, WB) | | |
| OTI3F9 | CD117 | KIT Monoclonal | ThermoFisher, | |
| | | Antibody (OTI3F9), | #TA801045 | |
| | | TrueMAB ™ | | |
| OTI4E4 | CD2 | available from multiple sources, including: | | |
| | | CD2 Monoclonal | ThermoFisher, | |
| | | Antibody (OTI4E4) | #TA500404 | |
| | | CD2 Mouse | Cambridge | |
| | | Monoclonal Antibody | Bioscience, | |
| | | [Clone ID: OTI4E4] | #TA500404 | |
| OTI4G8 | CD43 | ASPDH Monoclonal | ThermoFisher, | |
| | | Antibody (OTI4G8) | #MA5-27041 | |
| OTI6A4 | CD43 | PRRX1 Monoclonal | ThermoFisher, | |
| | | Antibody (OTI6A4) | #MA5-26579 | |
| OTI6F8 | CD117 | KIT Monoclonal | ThermoFisher, | |
| | | Antibody (OTI6F8), | #CF808746 | |
| | | TrueMAB ™ | | |
| OTI9A11 | CD117 | CD117 Mouse | OriGene, | |
| | | Monoclonal Antibody | #CF801205 | |
| | | [Clone ID: OTI9A11] | | |
| OX7 | CD90 | available from multiple sources, including: | | |
| | | CD90 Monoclonal | ThermoFisher, | |
| | | Antibody (OX-7) | #MA1-81491 | |
| | | Thy-1/CD90 Antibody | Santa Cruz | |
| | | (OX7) | Biotechnology, | |
| | | | #sc-53116 | |
| OX-45 | CD48 | CD48 Monoclonal | ThermoFisher, | |
| | | Antibody (OX-45), PE | #MA5-17528 | |
| OX-104 | CD200 | available from multiple sources, including: | | |
| | | CD200 Monoclonal | ThermoFisher, | |
| | | Antibody (OX104), | #14-9200-82 | |
| | | eBioscience ™ | | |
| | | CD200 antibody \| OX- | Bio-Rad, | |
| | | 104 | #MCA1960 | |
| pavurutamab | CD3 | Pavurutamab | MedChemExpress, | |
| | | | #HY-P99814 | |
| PD7/26 | CD45RB | CD45RB Monoclonal | ThermoFisher, | |
| | | Antibody (PD7/26), | #14-9458-82 | |
| | | eBioscience ™ | | |
| pembrolizumab | PD-1 | available from multiple sources, including: | | |
| | | Pembrolizumab | MedChemExpress, | |
| | | | #HY-P9902 | |
| | | | Evidentic, | |
| | | | #6302603D04 | |
| penpulimab | PD-1 | available from multiple sources, including: | | |
| | | Penpulimab | AbMole, | |
| | | | #M25087 | |
| | | Penpulimab (Anti- | #A3034 | |
| | | PDCD1/PD-1/CD279 | Selleckchem, | |
| peresolimab | PD-1 | Peresolimab | MedChemExpress, | |
| | | | #HY-P9993 | |
| PF-03475952 | CD44 | | | Runnels et al., 2010, |
| | | | | Adv. Ther. 27(3): |
| | | | | 168-180. |
| PG-M1 | CD68 | available from multiple sources, including: | | |
| | | Anti-CD68 antibody | Abcam, #ab783 | |
| | | [PG-M1] | | |
| | | CD68 Monoclonal | ThermoFisher, | |
| | | Antibody (PG-M1) | #MA5-12407 | |
| PI37012 | TREM2 | | | WO 2019/118513 |
| pidilizumab | PD-1 | Anti-Human PD- | Creative Biolabs, | |
| | | 1/PDCD1/CD279 | #TAB-900 | |
| | | Recombinant Antibody | | |
| | | (Pidilizumab) | | |
| pimivalimab | PD-1 | Pimivalimab | MedChemExpress, | |
| | | | #HY-P99887 | |
| pinatuzumab | CD22 | Pinatuzumab | MedChemExpress, | |
| | | | #HY-P99230 | |
| praluzatamab | CD166 | | | Boni et al., 2022, |
| | | | | Clin. Cancer Res. |
| | | | | 28(10): 2020-2029. |
| prezalumab | CD28 | Prezalumab | MedChemExpress, | |
| | | | #HY-P99414 | |
| priliximab | CD4 | available from multiple sources, including: | | |
| | | Priliximab | MedChemExpress, | |
| | | | #HY-P99790 | |
| | | Anti-Human CD4 | Creative Biolabs, | |
| | | Recombinant Antibody | #TAB-164 | |
| | | (Priliximab) | | |
| PRO1160 | CD70 | | | Jonasch et al., |
| | | | | 2023, Oncologist |
| | | | | 28(Suppl 1): S14- |
| | | | | S15. |
| prolgolimab | PD-1 | Prolgolimab | MedChemExpress, | |
| | | | #HY-P99807 | |
| promiximab | CD56 | Human Anti-NCAM1 | Creative Biolabs, | |
| | | Recombinant Antibody | #HPAB-NO187- | |
| | | | YC | |
| PRS-343 | CD137 | | | Hinner et al., 2016, |
| | | | | Cancer Immunol. |
| | | | | Res. 4 |
| | | | | (11_Supplement): |
| | | | | B016. |
| pucotenlimab | PD-1 | Pucotenlimab | MedChemExpress, | |
| | | | #HY-P99938 | |
| Q-1802 | CLDN18.2 | | | Yk et al., 2023, J. |
| | | | | Clin. Oncol. 41 (4 |
| | | | | suppl.), 382. |
| QBEND/10 | CD34 | CD34 Monoclonal | ThermoFisher, | |
| | | Antibody (QBEND/10), | #MA1-10205 | |
| | | PE | | |
| QL1706 | PD-1 | | | Zhang et al., 2021, |
| | CTLA-4 | | | Cancer Res. |
| | | | | 81(13_Suppl): |
| | | | | CT119. |
| quavonlimab | CTLA-4 | Quavonlimab | MedChemExpress, | |
| | | | #HY-P99809 | |
| r18D11 | CD14 | Anti-Human CD14 | Creative Biolabs, | |
| | | Recombinant Antibody | #TAB-1583CL | |
| | | (18D11) | | |
| ragifilimab | GITR | Ragifilimab | MedChemExpress, | |
| | | | #HY-P99812 | |
| RAVB3 | CD8 | available from multiple sources, including: | | |
| | | CD8 alpha Monoclonal | ThermoFisher, | |
| | | Antibody (RAVB3) | #MA5-17048 | |
| | | CD8 (RAVB3) | Santa Cruz | |
| | | | Biotechnology, | |
| | | | #sc-32812 | |
| RC118 | CLDN18.2 | | | Liu et al., 2024, Ann. |
| | | | | Oncol. 35 (Suppl. 2), |
| | | | | S903, 1456P. |
| REA101 | SSEA-4 | SSEA-4 Antibody, anti- | Miltenyi Biotec, | |
| | | human, PE, | #130-122-914 | |
| | | REAfinity ™ | | |
| REA157 | PODXL | TRA-1-60 Antibody, | Miltenyi Biotec, | |
| | | anti-human, PE, | #130-122-921 | |
| | | REAfinity ™ | | |
| REA246 | PODXL | TRA-1-81 Antibody, | Miltenyi Biotec, | |
| | | anti-human, PE, | #130-123-293 | |
| | | REAfinity ™ | | |
| REA233 | IL-21Rα | CD360 (IL-21R) | Miltenyi Biotec, | |
| | | Antibody, anti-human, | #130-126-094 | |
| | | PE, REAfinity ™ | | |
| REA333 | IL-12R | IL-12R β2 Antibody, | Miltenyi Biotec, | |
| | | anti-human, PE, | #130-120-068 | |
| | | REAfinity ™ | | |
| REA442 | CD166 | CD166 Antibody, anti- | Miltenyi Biotec, | |
| | | human, PE, | #130-118-349 | |
| | | REAfinity ™ | | |
| REA795 | SUSD2 | SUSD2 Antibody, anti- | Miltenyi Biotec, | |
| | | human, PE, | #130-111-641 | |
| | | REAfinity ™ | | |
| REA844 | CD271 | CD271 (LNGFR) | Miltenyi Biotec, | |
| | | Antibody, anti-human, | #130-112-601 | |
| | | PE, REAfinity ™ | | |
| REA877 | CD10 | CD10 Antibody, anti- | Miltenyi Biotec, | |
| | | human, pure, | #130-124-312 | |
| | | REAfinity ™ | | |
| REA897 | CD90 | CD90 Antibody, anti- | Miltenyi Biotec, | |
| | | human, PE, | #130-114-860 | |
| | | REAfinity ™ | | |
| REA1060 | CD29 | CD29 Antibody, anti- | Miltenyi Biotec, | |
| | | human, PE, | #130-118-121 | |
| | | REAfinity ™ | | |
| REA1067 | CD200 | CD200 Antibody, anti- | Miltenyi Biotec, | |
| | | human, PE, | #130-118-129 | |
| | | REAfinity ™ | | |
| REA1164 | CD34 | CD34 Antibody, anti- | Miltenyi Biotec, | |
| | | human, PE, | #130-120-515 | |
| | | REAfinity ™ | | |
| REAL219 | MSCA-1 | MSCA-1 Antibody, anti- | Miltenyi Biotec, | |
| | | human, PE, | #130-119-917 | |
| | | REAlease ® | | |
| REAL709 | CD271 | CD271 (LNGFR) | Miltenyi Biotec, | |
| | | Antibody, anti-human, | #130-125-053 | |
| | | PE, REAlease ® | | |
| REGN5459 | BCMA | | | U.S. Pat No. 11,384, 153 |
| REGN4659 | CTLA-4 | | | Burova et al., 2018, |
| | | | | Cancer Res. 78 |
| | | | | (13_Supplement): |
| | | | | 3824. |
| REGN6569 | GITR | | | Lakhani et al., 2022, |
| | | | | IOTECH 16 (S1): |
| | | | | 196TiP. |
| REGN7257 | CD132 | | | Le Floch-Ramondou |
| | | | | et al., 2022, Blood |
| | | | | 140 (Supplement 1): |
| | | | | 473-474. |
| relatlimab | LAG-3 | Relatlimab | MedChemExpress, | |
| | | | #HY-P99156 | |
| retifanlimab | PD-1 | Retifanlimab | MedChemExpress, | |
| | | | #HY-P99941 | |
| RG7356 | CD44 | Anti-Human CD44 | Creative Blolabs, | |
| | | Recombinant Antibody | #TAB-128CL | |
| | | (RG7356) | | |
| rituximab | CD20 | available from multiple sources, including: | | |
| | | BD Pharmingen ™ | BD Biosciences, | |
| | | Purified NA/LE Anti- | #569496 | |
| | | Human CD20 | | |
| | | Rituximab Biosimilar | | |
| | | HUMAN ANTI CD20 | BioRad, | |
| | | (RITUXIMAB | #MCA6091 | |
| | | BIOSIMILAR) | | |
| | | Rituximab | MedChemExpress, | |
| | | | #HY-P9913 | |
| RIV-11 | CD8 | available from multiple sources, including: | | |
| | | Anit-CD8 Antibody | Antibodies.com, | |
| | | (RIV-11) | #A115691 | |
| | | CD8 Recombinant | Aviva Systems | |
| | | Antibody( RIV-11) | Biology, | |
| | | | #OAAV00971 | |
| RM2051 | CD45 | Anti-CD45 antibody | Abcam, | |
| | | [RM2051] | #ab318154 | |
| RO5429083 | CD44 | | | Perez et al., 2012, |
| | | | | Cancer Res. 72 |
| | | | | (8_Suppl.): 2521. |
| RO7121661 | PD-1 | | | Clinical Trial ID: |
| | TIM-3 | | | NCT04785820 |
| RO7247669 | PD-1 | | | Clinical Trial ID: |
| | LAG-3 | | | NCT04785820 |
| RO7296682 | CD25 | Research Grade Anti- | AntibodySystem, | |
| | | Human CD25/IL2RA | #DHB95808 | |
| | | (RO7296682) | | |
| rocatinlimab | OX40 | Rocatinlimab | MedChemExpress, | |
| | | | #HY-P99955 | |
| RPA-2.10 | CD2 | available from multiple sources, including: | | |
| | | Purified anti-human | BioLegend, | |
| | | CD2 antibody | #300202 | |
| | | CD2 Monoclonal | ThermoFisher, | |
| | | Antibody (RPA-2.10) | #14-0029-82 | |
| | | Anti-Human CD2 | Stemcell | |
| | | Antibody, Clone RPA- | Technologies, | |
| | | 2.10 | #60007 | |
| RPA-T8 | CD8α | available from multiple sources, including: | | |
| (CT8) | | MOUSE ANTI HUMAN | Bio-Rad, | |
| | | CD8 | #MCA4609T | |
| | | CD8a Monoclonal | ThermoFisher, | |
| | | Antibody (RPA-T8), | #17-0088-42 | |
| | | APC | | |
| RTX-003 | CD25 | | | “Nature-inspired, |
| | | | | antibody discovery |
| | | | | driven by AI.” |
| | | | | Nature, December 2020, |
| | | | | B44. |
| rulonilimab | PD-1 | Rulonilimab | MedChemExpress, | |
| | | | #HY-P99895 | |
| RW03 | CD133 | | | Vora et al., 2020, |
| | | | | Cell Stem Cell |
| | | | | 26(6): 832-844.e6 |
| sabatolimab | TIM-3 | Sabatolimab | MedChemExpress, | |
| | | | #HY-P99044 | |
| samalizumab | CD200 | Samalizumab | MedChemExpress, | |
| | | | #HY-P99400 | |
| SAR445514 | BCMA | | | US 2024/0034816 |
| sasanlimab | PD-1 | Sasanlimab (Anti- | Selleckchem, | |
| | | PDCD1/PD-1/ | #A3038 | |
| | | CD279) | | |
| sdA1 | CD16a | | | WO 2018/039626 |
| sdA2 | CD16a | | | WO 2018/039626 |
| SEA-BCMA | BCMA | | | U.S. Pat No. 11,078,291 |
| selicrelumab | CD40 | Selicrelumab | MedChemExpress, | |
| | | | #HY-P99046 | |
| semzuvolimab | CD4 | Semzuvolimab | MedChemExpress, | |
| | | | #HY-P9998 | |
| serplulimab | PD-1 | Serplulimab (Anti- | Selleckchem, | |
| | | PDCD1/PD-1/ | #A2381 | |
| | | CD279) | | |
| SG001 | PD-1 | | | Clinical Trial ID: |
| | | | | NCT04886700 |
| SGN-CD70A | CD70 | | | Sandall et al., 2014, |
| | | | | Cancer Res. 74 |
| | | | | (19_Supplement): |
| | | | | 2647. |
| SHR-1702 | TIM-3 | | | Clinical Trial ID: |
| | | | | NCT03871855 |
| SHR-1802 | LAG-3 | | | Deng et al., 2023, |
| | | | | Ther. Adv. Med. |
| | | | | Oncol. 15: |
| | | | | 17588359231186025. |
| SIDI8BEE | CD8B | available from multiple sources, including: | | |
| | | CD8b Monoclonal | ThermoFisher, # | |
| | | Antibody (SIDI8BEE) | 14-5273-82 | |
| | | Mouse Anti-CD8B | CreativeBiolabs, | |
| | | Recombinant Antibody | #CBMAB- | |
| | | (SIDI8BEE) | C10732-LY | |
| sindelizumab | PD-1 | | | Li et al., 2022, Front. |
| | | | | Pharmacol. 13: |
| | | | | 918709. |
| sintilimab | PD-1 | Sintilimab | MedChemExpress, | |
| | | | #HY-P99048 | |
| siplizumab | CD2 | available from multiple sources, including: | | |
| | | Siplizumab | Medchem | |
| | | | Express, #HY- | |
| | | | P99904 | |
| | | Anti-Human CD2 | Creative Biolabs, | |
| | | Recombinant Antibody | #TAB-104 | |
| SJ25C1 | CD19 | | | Bejcek et al., 1995, |
| | | | | Cancer Res. |
| | | | | 55: 2346-2351 |
| SK1 | CD8α | available from multiple sources, including: | | |
| | | Purified Anti-human | AAT Bioquest, | |
| | | CD8 Antibody *SK1* | #10081000 | |
| | | Anti-CD8 Mouse | Avantor (VWR), | |
| | | Monoclonal Antibody | #344702-BL | |
| | | [clone: SK1] | | |
| SKB315 | CLDN18.2 | | | Clinical Trial ID: |
| | | | | NCT05367635 |
| SLAM.4 | CD150 | CD150 Monoclonal | ThermoFisher, | |
| | | Antibody (SLAM.4) | #MA5-46198 | |
| SM03 | CD22 | | | Wong et al., 2022, J. |
| | | | | Immunol. 208(12): |
| | | | | 2726-2737. |
| SOT102 | CLDN18.2 | | | Spisek, 2023, |
| | | | | ESMO Open 8(1), |
| | | | | Suppl. 2, 101196, |
| | | | | 2P. |
| sotigalimab | CD40 | Sotigalimab | MedChemExpress, | |
| | | | #HY-P99049 | |
| SP-16 | CD8 | available from multiple sources, including: | | |
| | | Anti-CD8 monoclonal | ThermoFisher, | |
| | | antibody (SP-16) | #MA5-14548 | |
| | | | Novus | |
| | | | Biologicals, | |
| | | | #NBP2-26484 | |
| SP34-2 | CD3 | BD Pharmingen ™ | BD Biosciences, | |
| | | APC-Cy ™7 Mouse | #557757 | |
| | | Anti-Human CD3 | | |
| SP55 | CD43 | CD43 Monoclonal | ThermoFisher, | |
| | | Antibody (SP55) | #MA5-16339 | |
| spartalizumab | PD-1 | Spartalizumab | MedChemExpress, | |
| | | | #HY-P9972 | |
| SPM504 | CD45RA | Anti-CD45RA antibody | Abcam, | |
| | | [SPM504] | #ab231426 | |
| SPX-101 | CLDN18.2 | | | Zhu et al., 2020, |
| | | | | Cancer Res. 80 |
| | | | | (16_Supplement): |
| | | | | 3361. |
| SR1 | CD117 | Anti-Human KIT | Creative Biolabs, | |
| | | Recombinant Antibody | #TAB-467LC | |
| | | (SR1) | | |
| ST04-99 | CD117 | CD117/c-kit Rabbit | Fisher Scientific, | |
| | | anti-Human, Clone: | #NBP267531 | |
| | | ST04-99, Novus | | |
| | | Biologicals ™ | | |
| STRO-1 | Stro-1 | STRO-1 Monoclonal | ThermoFisher, | |
| | | Antibody (STRO-1) | #39-8401 | |
| STRO-4 | Hsp90 | | | Gronthos et al., |
| | | | | 2009, Stem Cells |
| | | | | Dev. 18(9): 1253-62. |
| surzebiclimab | TIM-3 | Surzebiclimab | MedChemExpress, | |
| | | | #HY-P99962 | |
| Sym022 | LAG-3 | Research Grade Anti- | AntibodySystem, | |
| | | Human CD223/LAG3 | #DHD30423 | |
| | | (Sym022) | | |
| Sym023 | TIM-3 | | | Lindsted et al., |
| | | | | 2018, Cancer Res. 78 |
| | | | | (13_Supplement): |
| | | | | 5629. |
| Sym024 | CD73 | | | Jakobsen et al., |
| | | | | 2021, Cancer Res. 81 |
| | | | | (13_Supplement): |
| | | | | 1797. |
| SYSA180 | CLDN18.2 | | | Wang et al., 2023, J. |
| | | | | Clin. Oncol. 41 (16 |
| | | | | suppl.), 3016. |
| T200/797 | CD45RO | Anti-CD45RO antibody | Abcam, | |
| | | [T200/797] | #ab216024 | |
| T3-3A1 | CD7 | CD7 (T-Cell Leukemia | ThermoFisher, | |
| | | Marker) Monoclonal | #924-MSM5-PO | |
| | | Antibody (T3-3A1) | | |
| T6.3 | CD2 | available from multiple sources, including: | | |
| | | Mouse Anti-CD2 | Creative Biolabs, | |
| | | Recombinant Antibody | #ZG-0903F | |
| | | (clone T6.3) | | |
| | | Monoclonal Mouse | LS Bio, | |
| | | anti-Human CD2 | #LS-C140259 | |
| | | Antibody (clone T6.3) | | |
| T595 | CD117 | CD117 Mouse | OriGene, | |
| | | Monoclonal Antibody | #TA355261 | |
| | | [Clone ID: T595] | | |
| TAB-071CL | BMPR2 | Anti-Human BMPR | Creative Biolabs, | |
| | | Recombinant Antibody | #TAB-071CL | |
| talquetamab | GPRC5D | | | Pillarisetti et al., |
| | | | | Blood 135: 1232-43 |
| | | | | (2020) |
| tavolimab | OX40 | Tavolimab | ThermoFisher, | |
| | | Recombinant Human | #MA5-42300 | |
| | | Monoclonal Antibody | | |
| TB19 | CD73 | InVivoMAb Anti-Human | AntibodySystem, | |
| | | CD73/NT5E Antibody | #VHD46601 | |
| | | (TB19) | | |
| TB38 | CD73 | InVivoMAb Anti-Human | AntibodySystem, | |
| | | CD73/NT5E Antibody | #VHD46602 | |
| | | (TB38) | | |
| tebotelimab | LAG-3 | Tebotelimab | MedChemExpress, | |
| | PD-1 | | #HY-P99573 | |
| teclistamab | CD3 | Teclistamab | MedChemExpress, | |
| | | | #HY-P99392 | |
| telazorlimab | OX40 | Telazorlimab | MedChemExpress, | |
| | | | #HY-P99570 | |
| teplizumab | CD3 | Teplizumab | MedChemExpress, | |
| | | | #HY-P99222 | |
| TG6050 | CTLA-4 | | | Marchand et al., |
| | | | | 2023, Cancer Res. |
| | | | | 83 (7_Supplement): |
| | | | | 694. |
| TH-69 | CD7 | Anti-CD7 [TH69] | Absolute | |
| | | | Antibody, | |
| | | | #Ab03054 | |
| theralizumab | CD28 | Theralizumab | MedChemExpress, | |
| | | | #HY-P9975 | |
| tifcemalimab | BTLA | | | Song et al., 2023, |
| | | | | Blood 142 |
| | | | | (Supplement 1): |
| | | | | 4458. |
| tiragolumab | TIGIT | | | Kim et al., 2023, |
| | | | | JAMA Oncol. |
| | | | | 9(11): 1574-1582. |
| tisagenlecleucel | CD19 | | | Mueller et al., 2018, |
| | | | | Clin. Cancer Res. |
| | | | | 24 (24): 6175-6184. |
| tislelizumab | PD-1 | Tislelizumab | MedChemExpress, | |
| | | | #HY-P99052 | |
| TNB-383B | BCMA | | | U.S. Pat No. 11,505,606 |
| tocilizumab | IL-6R | available from multiple sources, including: | | |
| | | IL6R antibody \| rhPM-1 | BioRad, | |
| | | | #MCA6106 | |
| | | Tocilizumab | MedChemExpress, | |
| | | | #HY-P9917 | |
| | | Tocilizumab | ThermoFisher, | |
| | | Recombinant Human | #MA5-48061 | |
| | | Monoclonal Antibody | | |
| | | (rhPM-1 (Tocilizumab)) | | |
| toripalimab | PD-1 | Toripalimab | MedChemExpress, | |
| | | | #HY-P9978 | |
| TORL-2-307-ADC | CLDN18.2 | | | Clinical Trial ID: |
| | | | | NCT05156866 |
| tositumomab | CD20 | available from multiple sources, including: | | |
| | | Human CD20 | R&D Systems, | |
| | | (Research Grade | #FAB10440R | |
| | | Tositumomab | | |
| | | Biosimilar) Alexa | | |
| | | Fluor ® 647-conjugated | | |
| | | Antibody | | |
| | | Tositumomab | ThermoFisher, | |
| | | Recombinant Mouse | #MA5-41763 | |
| | | Monoclonal Antibody | | |
| TQB2618 | TIM-3 | Research Grade Anti- | AntibodySystem, | |
| | | Human CD366/HAVCR2/TIM-3 | #DHJ28406 | |
| | | (TQB2618) | | |
| TQB2934 | BCMA | | | US 2023/0193292 |
| TRC205 | CD105 | | | U.S. Pat No. 9,926,375 |
| tregalizumab | CD4 | Tregalizumab | MedChemExpress, | |
| | | | #HY-P99327 | |
| tremelimumab | CTLA-4 | Tremelimumab | MedChemExpress, | |
| | | | #HY-P9918 | |
| TRX1 | CD4 | Anti-Human CD4 | Creative Biolabs, | |
| | | Recombinant Antibody | #TAB-168LC | |
| | | (TRX1) | | |
| TRX2 | CD8 | Oxford Therapeutic Antibody Centre, | | |
| | | Oxford University, Oxford, United | | |
| | | Kingdom | | |
| TRX518 | GITR | Research Grade Anti- | AntibodySystem, | |
| | | Human | #DHJ89807 | |
| | | CD357/TNFRSF18/GITR | | |
| | | (TRX518) | | |
| TS1/8 | CD2 | available from multiple sources, including: | | |
| | | CD2 Monoclonal | ThermoFisher, | |
| | | Antibody (TS1/8) | #MA5-44080 | |
| | | Purified anti-human | BioLegend, | |
| | | CD2 Antibody | #309202 | |
| TS2/18 | CD2 | available from multiple sources, including: | | |
| | | CD2 Monoclonal | ThermoFisher, | |
| | | Antibody (TS2/18) | #MA0200 | |
| | | CD2 Antibody | Santa Cruz Bio, | |
| | | (TS2/18.1.1) | #sc-19640 | |
| TTI-CD200 | CD200 | | | Rastogi et al., 2021, |
| | | | | Br. J. Haematol. |
| | | | | 193, 155-159. |
| TUSP-2 | Stro-1 | Anti-STRO-1 | Creative | |
| | | monoclonal antibody | Diagnostics, | |
| | | [Alexa Fluor ® 647] | #DMAB18656 | |
| UB-421 | CD4 | Research Grade | AntibodySystem, | |
| | | Semzuvolimab | #DHB95908 | |
| ublituximab | CD20 | available from multiple sources, including: | | |
| | | Ublituximab | MedChemExpress, | |
| | | | #HY-P99538 | |
| | | Ublituximab (Anti- | SelleckChem, | |
| | | CD20) | #A2807 | |
| | | Ublituximab | ThermoFisher, | |
| | | Recombinant Human | #MA5-41938 | |
| | | Monoclonal Antibody | | |
| UCART20 | CD20 | | | Kang et al., 2024, J. |
| | | | | Hem. Oncol. 17: 29. |
| UCHL1 | CD45RO | Anti-CD45RO antibody | Abcam, #ab23 | |
| | | [UCH-L1] | | |
| UCHT2 | CD5 | CD5 Monoclonal | ThermoFisher, | |
| | | Antibody (UCHT2), | #14-0059-82 | |
| | | eBioscience ™ | | |
| UCHT4 | CD8 | available from multiple sources, including: | | |
| | | CD8 Monoclonal | ThermoFisher, # | |
| | | Antibody (UCHT4) | 65204-1-IG | |
| | | Anti-CD8 [UCHT4] | Absolute | |
| | | | Antibody, | |
| | | | #Ab00694-2.0 | |
| uliledlimab | CD73 | Uliledlimab | MedChemExpress, | |
| | | | #HY-P99169 | |
| UMAB216 | CD117 | KIT Monoclonal | ThermoFisher, | |
| | | Antibody (UMAB216), | #UM800108CF | |
| | | UltraMAB ™ | | |
| UMCD2 | CD2 | available from multiple sources, including: | | |
| | | anti-human CD2 | Biohippo, | |
| | | | #BHA14700014 | |
| | | CD2 Monoclonal | Biotium, #1235 | |
| | | Mouse Antibody | | |
| | | (UMCD2) | | |
| urelumab (BMS- | CD137 | Urelumab | MedChemExpress, | |
| 663513) | | | #HY-P99055 | |
| ustekinumab | IL-12 | available from multiple sources, including: | | |
| | IL-23 | Ustekinumab Biosimilar - | Proteogenix, | |
| | | Anti-Human IL-12 IL- | #PX-TA1011 | |
| | | 23 mAb - Research | | |
| | | Grade | | |
| | | Ustekinumab (anti-IL- | SelleckChem, | |
| | | 12/IL-23) | #A2024 | |
| utomilumab | CD137 | Utomilumab | MedChemExpress, | |
| (PF-05082566) | | | #HY-P99056 | |
| varlilumab | CD27 | Varlilumab | ThermoFisher, | |
| | | Recombinant Human | #MA5-42030 | |
| | | Monoclonal Antibody | | |
| VB6-008 | CD44 | | | U.S. Pat No. 8,383,117 |
| veltuzumab | CD20 | available from multiple sources, including: | | |
| | | Veltuzumab | MedChemExpress, | |
| | | | #HY-P99224 | |
| | | Veltuzumab Biosimilar - | Proteogenix, | |
| | | Anti- CD20 receptors | #PX-TA1616 | |
| | | mAb - Research | | |
| | | Grade | | |
| | | Veltuzumab | ThermoFisher, | |
| | | Recombinant Human | #MA5-42301 | |
| | | Monoclonal Antibody | | |
| VIB9600 | CD32A | | | Chen et al., 2019, |
| | | | | Ann. Rheum. Dis. |
| | | | | 78(2): 228-237. |
| vibecotamab | CD3 | Vibecotamab | Fisher Scientific, | |
| | | Recombinant Human | #PIMA542248 | |
| | | Monoclonal Antibody, | | |
| | | Invitrogen ™ | | |
| vibostolimab | TIGIT | | | Niu et al., 2022, |
| (MK-7684) | | | | Ann. Oncol. 33(2): |
| | | | | 169-180. |
| visilizumab | CD3 | Anti-CD3E | Creative Biolabs, | |
| | | Recombinant Antibody | #TAB-714 | |
| | | (Visilizumab) | | |
| vonlerizumab | OX40 | Vonlerizumab (Anti- | SelleckChem, | |
| | | TNFRSF4/OX40/ | #A2325 | |
| | | CD134) | | |
| vorsetuzumab | CD27 | Vorsetuzumab | MedChemExpress, | |
| | CD70 | | #HY-P99273 | |
| vudalimab | PD-1 | Research Grade | AntibodySystem, | |
| | | Vudalimab | #DHH02222 | |
| vunakizumab | CD27 | Anti-Human IL 17A | Creative Biolabs, | |
| | | Recombinant Antibody | #TAB-458CQ | |
| | | (Vunakizumab) | | |
| W3/13HLK | CD43 | CD43 Monoclonal | ThermoFisher, | |
| | | Antibody (W3/13HLK), | #MA5-17386 | |
| | | PE | | |
| W5C5 | SUSD2 | Purified anti-human | BioLegend, | |
| | | SUSD2 Antibody | #327401 | |
| W8B2 | MSCA-1 | MSCA-1 Antibody, anti- | Miltenyi Biotec, | |
| | | human | #130-093-595 | |
| WT1 | CD7 | Mouse Anti-CD7 | Creative Biolabs, | |
| | | Recombinant Antibody | #CBMAB- | |
| | | (WT1) | C10581-LY | |
| WV078 | BCMA | | | U.S. Pat No. 11,492,409 |
| X9C3 | MSCA-1 | Mouse anti-Human | Creative | |
| | | MSCA-1 monoclonal | Diagnostics, | |
| | | antibody, PE | #CABT-B12374 | |
| XTX101 | CTLA-4 | | | Jenkins et al., 2023, |
| | | | | J. Immunother. |
| | | | | Cancer 11(12): |
| | | | | e007785. |
| Y1/82A | CD68 | CD68 antibody \| | Bio-Rad, | |
| | | Y1/82A | #MCA6014GA | |
| YB5.B8 | CD117 | CD117 (c-Kit) | ThermoFisher, | |
| | | Monoclonal Antibody | #14-1179-82 | |
| | | (YB5.B8), | | |
| | | eBioscience ™ | | |
| YH001 | CTLA-4 | | | Ganju et al., 2021, |
| | | | | J. Clin. Oncol. 39 |
| | | | | (15_suppl): 2577. |
| YTC182.20 | CD8 | Anti-CD8 alpha | Abcam, | |
| | | antibody [YTC182.20] | #ab60076 | |
| YTH3.2.6 | CD7 | Anti-CD7 [YTH3.2.6] | Absolute | |
| | | | Antibody, | |
| | | | #Ab01019 | |
| zalifrelimab | CTLA-4 | Zalifrelimab | MedChemExpress, | |
| | | | #HY-P99514 | |
| zanolimumab | CD4 | Zanolimumab | ThermoFisher, | |
| | | Recombinant Human | #MA5-41819 | |
| | | Monoclonal Antibody | | |
| zimberelimab | PD-1 | Zimberelimab | MedChemExpress, | |
| | | | #HY-P99109 | |
| zolbetuximab | CLDN18.2 | | | Sahin et al., 2018, |
| (IMAB362) | | | | Eur. J. Cancer 100, |
| | | | | 17-26. |

1. An ionizable cationic lipid having a structure of formula M2, 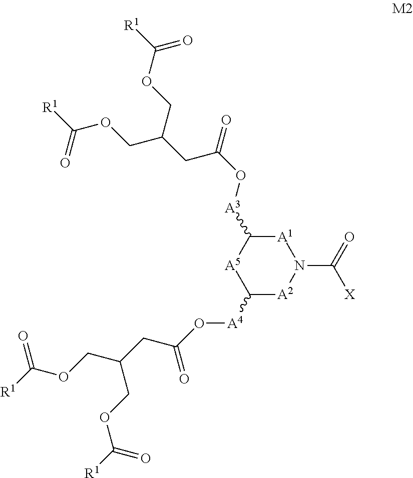
wherein X is 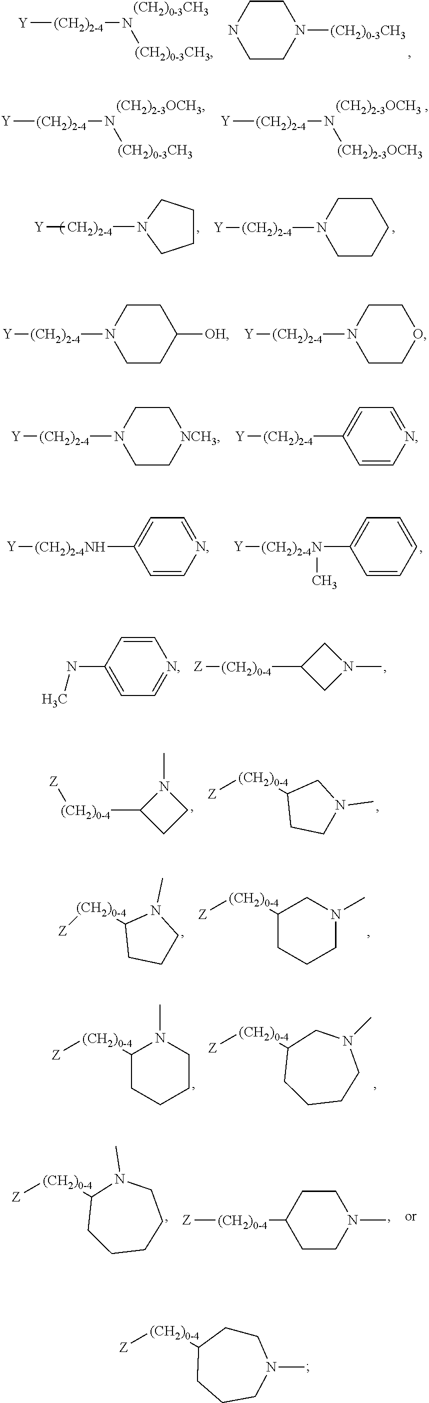
Y is O, S, NH, or NCH₃;
Z is O, NH, or NCH₃;
each R¹ is independently selected from C₇-C₁₁ alkyl or C₇-C₁₁ alkenyl; and
A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂; or
A¹ is (CH₂)₀, A² is (CH₂)₁, A³ is (CH₂)₁, A⁴ is (CH₂)₀, and A⁵ is (CH₂)₁; or
A¹ is (CH₂)₁, A² is (CH₂)₁, A³ is (CH₂)₀, A⁴ is (CH₂)₀, and A⁵ is (CH₂)₀; or
A¹ is (CH₂)₁, A² is (CH₂)₁, A³ is (CH₂)₀, A⁴ is (CH₂)₀, and A⁵ is (CH₂)₁; or
A¹ is (CH₂)₁, A² is (CH₂)₁, A³ is (CH₂)₀, A⁴ is (CH₂)₀, and A⁵ is (CH₂)₂ or CH═CH, and
wherein a wavy bond indicates that any relative or absolute stereo-configuration of the corresponding ring atom, or a mixture of stereo-configurations, can be assumed.

2. The ionizable cationic lipid of claim 1, wherein A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

3. The ionizable cationic lipid of claim 1, wherein A¹ is (CH₂)₀, A² is (CH₂)₁, A³ is (CH₂)₁, A⁴ is (CH₂)₀, and A⁵ is (CH₂)₁.

4. The ionizable cationic lipid of claim 1, wherein A¹ is (CH₂)₁, A² is (CH₂)₁, A³ is (CH₂)₀, A⁴ is (CH₂)₀, and A⁵ is (CH₂)₀.

5. The ionizable cationic lipid of claim 1, wherein A¹ is (CH₂)₁, A² is (CH₂)₁, A³ is (CH₂)₀, A⁴ is (CH₂)₀, and A⁵ is (CH₂)₁.

6. The ionizable cationic lipid of claim 1, wherein A¹ is (CH₂)₁, A² is (CH₂)₁, A³ is (CH₂)₀, A⁴ is (CH₂)₀, and A⁵ is (CH₂)₂ or CH═CH.

7. The ionizable cationic lipid of claim 1, wherein R¹ is (CH₂)₇CH₃.

8. The ionizable cationic lipid of claim 1, having the structure: 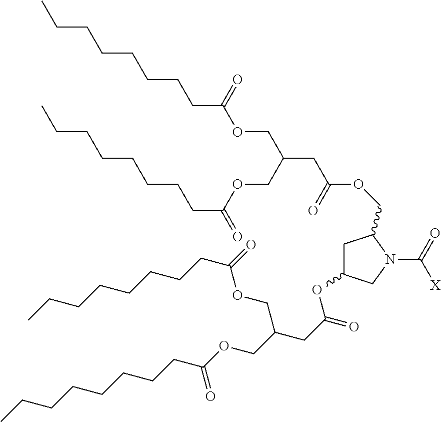
wherein the wavy bonds indicate that any relative or absolute stereo-configuration of the corresponding ring atom, or a mixture of stereo-configurations, can be assumed.

9. The ionizable cationic lipid of claim 8, having the structure CICL-207:

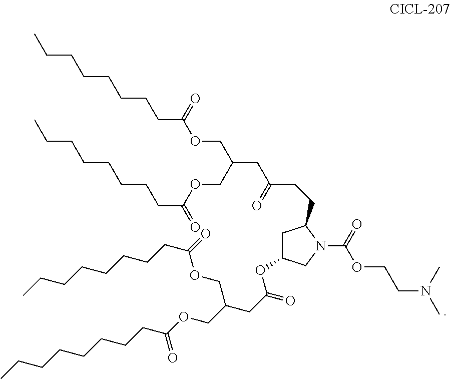 [SMILES: CCCCCCCCC(=O)OCC(COC(=O)CCCCCCCC)CC(=O)CC[C@@H]1C[C@@H](OC(=O)CC(COC(=O)CCCCCCCC)COC(=O)CCCCCCCC)CN1C(=O)OCCN(C)C]

10. The ionizable cationic lipid of claim 8 having the structure CICL-225:

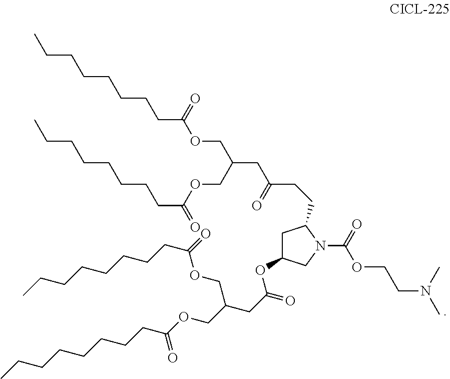 [SMILES: CCCCCCCCC(=O)OCC(COC(=O)CCCCCCCC)CC(=O)CC[C@H]1C[C@H](OC(=O)CC(COC(=O)CCCCCCCC)COC(=O)CCCCCCCC)CN1C(=O)OCCN(C)C]

11. The ionizable cationic lipid of claim 1, having the structure: 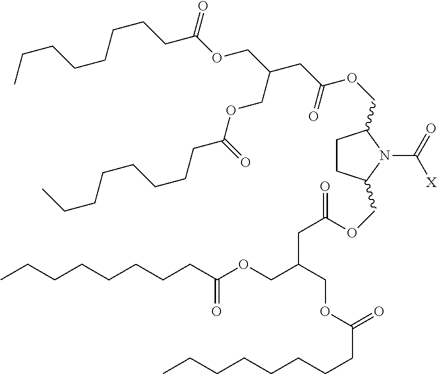
wherein the wavy bonds indicate that any relative or absolute stereo-configuration of the corresponding ring atom, or a mixture of stereo-configurations, can be assumed.

12. The ionizable cationic lipid of claim 1, having the structure: 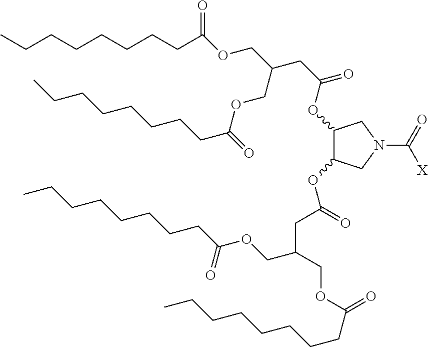
wherein the wavy bonds indicate that any relative or absolute stereo-configuration of the corresponding ring atom, or a mixture of stereo-configurations, can be assumed.

13. The ionizable cationic lipid of claim 12 having the structure CICL-215: 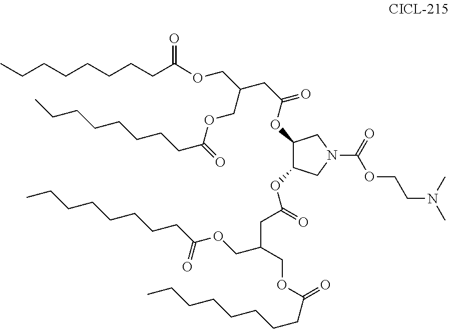 [SMILES: CCCCCCCCC(=O)OCC(COC(=O)CCCCCCCC)CC(=O)O[C@H]1CN(C(=O)OCCN(C)C)C[C@@H]1OC(=O)CC(COC(=O)CCCCCCCC)COC(=O)CCCCCCCC]
or its enantiomer.

14. The ionizable cationic lipid of claim 1, wherein X is

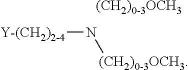

15. The ionizable cationic lipid of claim 11, wherein X is

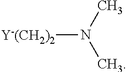

16. The ionizable cationic lipid of claim 12, wherein Y is O.

17. The ionizable cationic lipid of claim 1, wherein Z is O.

18. A lipid nanoparticle (LNP), comprising at least one ionizable cationic lipid of claim 1.

19. The LNP of claim 18, further comprising one or more of a phospholipid, a sterol, a co-lipid, an unfunctionalized PEG-lipid, a functionalized PEG-lipid, or combinations thereof.

20. The LNP of claim 19, comprising at least one phospholipid, wherein the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof.

21. The LNP of claim 19, comprising at least one sterol, wherein the sterol comprises cholesterol, campesterol, sitosterol, stigmasterol, or combinations thereof.

22. The LNP of claim 19, comprising at least one unfunctionalized PEG-lipid or at least one functionalized PEG-lipid, wherein the unfunctionalized PEG-lipid or functionalized PEG-lipid comprises a PEG moiety of 1000-5000 Da molecular weight (MW).

23. The LNP of claim 19, comprising at least one unfunctionalized PEG-lipid or at least one functionalized PEG-lipid, wherein the unfunctionalized PEG-lipid or functionalized PEG-lipid comprises fatty acids with a fatty acid chain length of C₁₄-C₁₈.

24. The LNP of claim 19, wherein the at least one ionizable cationic lipid is present in an amount in the range from about 40 to about 65 mol %.

25. The LNP of claim 19, comprising a phospholipid in an amount in the range from 7 to 30 mol %, a sterol in an amount in the range from about 20 to about 45 mol %, at least one co-lipid in an amount in the range from about 1 to about 30 mol %, at least one unfunctionalized PEG-lipid in an amount in the range from about 0.1 to about 5 mol %, or at least one functionalized PEG-lipid in an amount in the range from about 0.1 to about 5 mol %, or any combination thereof.

26. A targeted lipid nanoparticle (tLNP), comprising at least one ionizable cationic lipid of claim 1 and a functionalized PEG-lipid, wherein the functionalized PEG-lipid has been conjugated with a binding moiety.

27. The tLNP of claim 26, wherein the binding moiety comprises an antigen, a ligand-binding domain of a receptor, a receptor ligand, or an antigen binding domain of an antibody or antigen binding fragment thereof.

28. A lipid nanoparticle (LNP) or targeted lipid nanoparticle (tLNP), comprising at least one ionizable cationic lipid of claim 1 and comprising a biologically active payload nucleic acid.

29. A method of delivering a nucleic acid into a cell comprising contacting the cell with the LNP or the tLNP of claim 28.

30. A lipid nanoparticle (LNP) or targeted lipid nanoparticle (tLNP), comprising at least one ionizable cationic lipid having the structure CICL-207, CICL-215, or CICL-225.


SEQUENCE LISTING (6 sequences)

SEQ ID NO: 1 - protein, 450 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - RNA, 1776 nt, synthetic construct
  source 1..1776: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 1969 nt, synthetic construct
  source 1..1969: mol_type = other RNA
agcataaaagtcacatttgcttctgacatagttgtgttgactcacaaccccagaaacagacatcgaattcgctgccaccatggaagacgccaaaaacataaagaaaggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaaaagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggaaagatcgccgtgtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - RNA, 1128 nt, synthetic construct
  source 1..1128: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggtgtccaagggcgaggaagataatatggccatcatcaaggagttcatgaggttcaaggtgcacatggaaggcagcgttaacggccacgagttcgagatcgagggcgaaggagaaggaagaccctacgagggaacacagaccgctaaactgaaagtgacaaagggaggccctctgccattcgcctgggacatcctgtcccctcagttcatgtacggcagcaaggcctacgtcaagcatcctgccgacatccctgactacctgaagctgagctttcccgagggcttcaagtgggaacgggtgatgaacttcgaggacggcggcgtggtgaccgtgacacaagacagcagcctgcaggatggagagtttatttataaggtgaagctgagaggcaccaacttccccagcgacggccctgtgatgcagaaaaagaccatgggctgggaggcctctagcgagcggatgtatcctgaagatggcgccctgaagggcgagatcaagcagagactgaaactgaaggacggcggccactacgacgccgaggtgaaaaccacctacaaggccaagaagcctgtgcagctgcccggcgcttacaacgtgaatatcaagctcgatatcaccagccacaacgaggattacaccatcgtggaacagtacgagagagccgaaggcagacactctacaggaggcatggacgagctgtacaagtgaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 6 - protein, 5 aa, synthetic construct
LPXTG